(19) United States
(12) Patent Application Publication
(10) Pub. No.: US 2023/0002779 A1
(43) Pub. Date: Jan. 5, 2023

(54) CHARACTERIZATION OF PLANT CULTIVARS BASED ON TERPENE SYNTHASE GENE PROFILES

(71) Applicant: Front Range Biosciences, Inc., Boulder, CO (US)

(72) Inventors: Christopher Stephen PAULI, Boulder, CO (US); Anthony TORRES, Oakland, CA (US); Reginald GAUDINO, San Marcos, CA (US); Keith ALLEN, Port Ludlow, WA (US); Thomas BLANK, Palmetto, FL (US); Kymron DE CESARE, Los Gatos, CA (US)

(21) Appl. No.: 17/361,187

(22) Filed: Jun. 28, 2021

Related U.S. Application Data
(60) Provisional application No. 63/045,604, filed on Jun. 29, 2020.

(51) Int. Cl. C12N 15/82

(52) U.S. Cl. CPC C12N 15/8245

(57) ABSTRACT

This technology relates in part to methods of identifying plant cultivars based on the terpene synthase genes that are identified and/or quantified (e.g., copy number, ploidy) in the cultivars. The methods provided herein permit plant cultivars with desired characteristics/phenotypes, e.g., a desired terpene production profile, to be selected for use in various applications, such as agriculture (e.g., selecting cultivars for breeding desired characteristics and/or lineages) and medicine (e.g., therapeutic activity).

### RELATED PATENT APPLICATION

This patent application claims priority to U.S. Provisional Patent Application No. 63/045,604 filed on Jun. 29, 2020, entitled CHARACTERIZATION OF PLANT CULTIVARS BASED ON TERPENE SYNTHASE GENE PROFILES, naming Christopher Stephen PAULI et al. as inventors, and designated by Attorney Docket No. FRB-1002-PV. The entire content of the foregoing patent application is incorporated herein by reference for all purposes.

### SEQUENCE LISTING

The instant application contains a Sequence Listing which has been submitted electronically in ASCII format and is hereby incorporated by reference in its entirety. Said ASCII copy, created on Sep. 16, 2021, is named FRB-1002-UTt_SL.txt and is 450,572 bytes in size.

### FIELD

The technology relates in part to methods and compositions for detecting one or more terpene synthase genes and/or paralogs thereof in plants, thereby obtaining terpene synthase gene profiles. These profiles can be used to identify plant cultivars of a desired phenotype for agricultural, medicinal or industrial use.

### BACKGROUND

Terpenes are the largest and most structurally diverse class of natural compounds. They are produced by a large variety of plants, fungi, bacteria, and a few insects. To date, around 50,000 terpenoid metabolites, including monoterpenes, sesquiterpenes, and diterpenes, have been identified from higher plants, liverworts, and fungi. Terpenes play central roles in plant communication with the environment, including attracting beneficial organisms, repelling harmful ones, and facilitating communication between plants. The diversity of terpenoid compounds produced by plants plays an important role in mediating plant—herbivore, plant—pollinator, and plant—pathogen interactions. In plants such as Cannabis cultivars (e.g., Cannabis sativa), monoterpenes and sesquiterpenes are also responsible for most of their odor and flavor properties. Thus, variation in terpene content can be an important differentiator between cultivars of plants.

Terpene synthases (TPSs) are the key enzymes responsible for the biosynthesis of terpenes. Angiosperms, such as Cannabis, tend to have moderately large families of these enzymes, some apparently from recent duplications (e.g., paralogous enzymes), and others quite distant from each other, with both divergent and convergent evolution taking place. Thus, some TPS enzymes are highly divergent in sequence, while others (e.g., paralogs) differ from existing enzymes by just a few amino acids. Regardless, in general, the product profile of a given TPS enzyme cannot readily be determined from sequence similarity with or differences from other TPS gene family members. Therefore, for a given plant cultivar, there is a need to reliably identify all the members of the TPS gene family that are present, regardless of whether they are similar or different in sequence, in order to characterize the terpene production profile of a plant cultivar.

### SUMMARY

Provided herein are methods of analyzing a plant cultivar containing at least one terpene synthase gene or a paralog thereof, that include:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique subsequence of the terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene or a paralog thereof is different than the subsequences of other terpene synthase genes and/or paralogs thereof;
(c) amplifying the mixture, thereby obtaining an amplified mixture; and
(d) analyzing the amplified mixture of (c), whereby at least one terpene synthase gene or a paralog thereof is identified and/or quantified in the amplified mixture.

The terpene synthase (TPS) genes or paralogs thereof analyzed by any of the methods provided herein can have sequence identity at percentages from between about 40% to about 90, 91, 92, 93, 94, 95, 96, 97, 98, 99 or 100%, such as at least 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. In certain embodiments, the TPS gene is a paralog of another TPS gene. As used herein, the term “paralog” refers to members of a family of genes, such as a family of TPS genes, that share a high degree of overall sequence identity, generally at least about 80%, or about 80, 81, 82, 83, 84, 85, 86, 87, 88, 89, 90, 91, 92, 93, 94, 95, 96, 97, 98 or 99% or more sequence identity, but less than 100% sequence identity. In embodiments, the TPS paralogs share 90, 91, 92, 93, 94, 95, 96, 97, 98, 99, 99.1, 99.2, 99.3, 99.4, 99.5, 99.6, 99.7, 99.8 Or 99.9% or more sequence identity, but less than 100% sequence identity. Paralogs of a gene, such as a TPS gene, can sometimes arise by duplication within one species. While paralogs of a gene can have the same function and share a high degree of sequence identity, the paralogs often diverge and develop differences in function. For example, paralogs of TPS that are analyzed by the methods provided herein can have differences in their function, such as differing in the amount of a terpene that is generated by catalysis with each paralog, or the type of terpene generated, or the number of terpene products that are generated. A family of genes, as referred to herein, means genes that perform the same function, e.g., catalyzing the synthesis of terpenes, but are involved in producing different terpenes or sets of terpenes. For example, the TPS genes can include monoterpene synthases, diterpene synthases, sesquiterpene synthases and paralogs thereof. Within each of the classes of TPS genes, the types and/or combinations of terpenes (monoterpenes, diterpenes, sesquiterpenes) that are produced can vary.

The unique subsequence of a terpene synthase gene (TPS), such as an exon or a portion thereof, or an intron or a portion thereof, can differ from other subsequences of the terpene synthase gene or from subsequences of other terpene synthase genes by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 or more bases, such as 110, 120, 130, 140 or 150 or more bases. In embodiments of the methods provided herein, each primer pair specifically hybridizes to a unique subsequence of a TPS gene. In the methods provided herein, identifying and/or quantifying the TPS genes of a plant cultivar based on amplifying unique subsequences of the TPS genes, such as an exon, an intron or a portion thereof that is uniquely present in one TPS gene and not in any other TPS gene, the TPS gene profile of a plant cultivar can be obtained regardless of the overall sequence identity among the TPS genes in the profile, including when the overall sequence identity among certain TPS genes is high (e.g., paralogs). the other subsequences of the terpene synthase gene or a paralog thereof and the unique subsequence of the terpene synthase gene or a paralog thereof is different than the subsequences of other terpene synthase genes and/or paralogs thereof;

In embodiments, provided herein is a method of preparing nucleic acid containing at least one terpene synthase gene or a paralog thereof from a plant cultivar comprising the at least one terpene synthase gene or a paralog thereof, that include:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique subsequence of the terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene is different than the subsequences of the other terpene synthase genes and/or paralogs thereof;
(c) amplifying the mixture, thereby obtaining an amplified mixture; and
(d) analyzing the amplified mixture of (c), whereby at least one terpene synthase gene or a paralog thereof is identified and/or quantified in the amplified mixture.

In certain embodiments, the plant cultivar contains a plurality of terpene synthase genes and/or paralogs thereof and the method includes:
in (b), contacting the nucleic acid sample with a plurality of polynucleotide primer pairs under amplification conditions, thereby preparing a mixture, wherein each of the plurality of polynucleotide primer pairs hybridizes to a unique subsequence of a terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene or a paralog thereof is different than the subsequences of the other terpene synthase genes and/or paralogs thereof; and
in (d), a plurality of terpene synthase genes and/or paralogs thereof are identified and/or quantified.

In certain embodiments, each of the primers of a polynucleotide primer pair hybridizes to a conserved region of the subsequence and the hybridized polynucleotide primer pair flanks a variable region of the subsequence. In embodiments, the subsequence contains an exon, an intron, a portion within an exon or a portion within an intron. In certain embodiments, the subsequence is an exon or a portion within an exon.

In embodiments of the methods provided herein, the identification in (d) is by one or more of high-resolution melting (HRM), quantitative PCR (qPCR), loop-mediated isothermal amplification (LAMP), restriction endonuclease digestion, gel electrophoresis and sequencing. In certain embodiments, the number of TPS genes and/or paralogs thereof that are analyzed according to the methods provided herein can be from a single TPS gene to 100 or more TPS genes, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 or more TPS genes or paralogs of TPS genes.

In certain embodiments of the methods provided herein, one or more of the polynucleotide primer pairs are selected from among those set forth in SEQ ID NOS: 1-1284. In embodiments of the methods provided herein, one or more of the polynucleotide primer pairs are selected from among those set forth in SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284. In embodiments, any of the forward primers in the primer pairs provided in SEQ ID NOS: 1-1284, or in sequences that share 90%, 91%, 92%, 93%, 94%, 95%, 96%, 97%, 98%, 99% or more identity with any of the sequences set forth in SEQ ID NOS: 1-1284, can be paired with any of the reverse primers of the primer pairs having the sequences set forth in SEQ ID NOS: 1-1284, or in sequences that share 90%, 91%, 92%, 93%, 94%, 95%, 96%, 97%, 98%, 99% or more identity with any of the sequences set forth in SEQ ID NOS: 1-1284.

In embodiments, at least one terpene synthase is a paralog of a terpene synthase gene. The terpene synthase genes and/or paralogs thereof that are identified and/or quantified by the methods provided herein can, in certain embodiments, be selected from among monoterpene synthase genes and/or paralogs thereof, diterpene synthase genes and/or paralogs thereof, sesquiterpene synthase genes and/or paralogs thereof, or any combination thereof. In embodiments, based on the terpene synthase genes and/or paralogs thereof that are identified and/or quantified, the terpene synthase gene and/or paralog expression profile and/or the terpene production profile of the plant cultivar is determined. In certain embodiments, the terpene synthase gene and/or paralog expression profile and/or the terpene production profile is of the root, flower, stem, leaf or any combination thereof.

In certain embodiments of the methods provided herein, based on the terpene synthase genes and/or paralogs thereof that are identified and/or quantified and/or based on the terpene synthase gene expression profile that is determined and/or based on the terpene production profile that is determined, a lineage of the plant cultivar is assigned. In certain embodiments, based on the assigned lineage, the plant is selected for cultivation as a crop, or for in-breeding or out-crossing. Also provided herein are methods of cultivating crops by selecting a plant cultivar based on its lineage. Also provided herein are methods of breeding a plant cultivar that is selected based on its lineage. In certain embodiments, offspring plant cultivars can be bred for a desired terpene synthase gene expression profile, terpene content or cannabinoid content by tracking the terpene synthase genes in the parent plant cultivars.

In embodiments, based on the terpene synthase genes and/or paralogs thereof that are identified and/or quantified and/or based on the terpene synthase gene and/or paralog thereof expression profile that is determined and/or based on the terpene production profile that is determined, a medicinal use of the plant cultivar is assigned. In embodiments, the medicinal use can be selected from among antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects, or any combinations thereof. In certain embodiments, a plant cultivar is selected for a desired medicinal use. In embodiments, provided herein are methods of treatment comprising administering a plant cultivar, or portion thereof (e.g., seed, root, stem, flower) or an extract thereof (e.g., extracts from tissues of the plant cultivar) to a subject having a condition or disease in need of such treatment, whereby the condition or disease, or symptoms thereof, are ameliorated or reduced. In certain embodiments, based on the medicinal use, the plant is selected for cultivation as a crop, or for in-breeding or out-crossing. Also provided herein are methods of cultivating crops by selecting a plant cultivar based on its medicinal use. Also provided herein are methods of breeding a plant cultivar that is selected based on its medicinal use.

In embodiments, based on the terpene synthase genes and/or paralogs thereof that are identified and/or quantified and/or based on the terpene synthase gene and/or paralog thereof expression profile that is determined and/or based on the terpene production profile that is determined, the plant cultivar is identified as resistant to an organism or situation, or as having an affinity towards or favoring an organism or situation. In certain embodiments, the organism or situation is selected from among insects, pests, mold, pesticides and other chemicals, mildew, fungi, bacteria, viruses or other pathogens, an environmental condition, such as climate or soil conditions, or a geographic location.

In embodiments of any of the methods provided herein, a plurality of plant cultivars can be analyzed. In certain embodiments, the plant cultivars are of the same species. In embodiments, the plurality of plant cultivars can be classified based on lineage and/or based on medicinal use. In certain embodiments of the methods provided herein, one or more plant cultivars is/are a Cannabis cultivar. In aspects, the Cannabis cultivar is selected from among one or more of Type 1, Type 2, Type 3, Type 4 and Type 5 cultivars. In embodiments, one or more plant cultivars are of the family Rosidae.

In certain embodiments, the monoterpene synthase genes and/or paralogs thereof of the Cannabis plant cultivar are identified and/or quantified and, based on the identified and/or quantified monoterpene synthase genes and/or the expression profile of the identified and/or quantified monoterpene synthase genes and/or paralogs thereof, the terpene production profile, the cannabinoid production profile, the flavonoid production profile, or any combination of two or more of the terpene production profile, the cannabinoid production profile and the flavonoid production profile of the Cannabis plant cultivar is determined.

In embodiments, based on the terpene synthase genes and/or paralogs thereof of the Cannabis plant cultivar that are identified and/or quantified, or based on the expression profile of the identified and/or quantified terpene synthase genes and/or paralogs thereof, or based on the terpene production profile, or based on the cannabinoid production profile, or based on the flavonoid production profile, or based on any combination thereof, a lineage of the Cannabis plant cultivar is assigned, and/or a medicinal use of the plant cultivar is assigned. In certain embodiments, a plurality of Cannabis plant cultivars are analyzed and in embodiments, the plurality of Cannabis plant cultivars are further classified based on lineage and/or based on medicinal use.

In any of the methods provided herein, in certain embodiments, at least one plant cultivar that is analyzed produces one or more terpenes selected from among 60 -Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, γ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In embodiments, the at least one plant cultivar includes terpene synthases that produce, singly or as combinations of terpene synthases, one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, αeudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In certain embodiments of the methods provided herein, a terpene production profile is determined for one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In embodiments of the methods provided herein, at least one plant cultivar that is analyzed expresses one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, a terpene synthase expression profile is determined for one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, the one or more terpene synthases are selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPSSJL, TPSSJL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

In certain embodiments of the methods provided herein, the plant cultivar is a Cannabis cultivar selected from among Cannabis sativa, a Cannabis indica, or Cannabis ruderalis, such as Jamaican Lion (JL), Purple Kush (PK), CannaTsu (CT), Finola (FN), Valley Fire (VF), Cherry Chem (CC), LPA004 (L4), and LPA021.3 (L21). Examples of Cannabis genomes include CS10, Arcata Trainwreck, Grape Stomper, Citrix, Black 84, Headcheese, Red Eye OG, Tahoe OG, Master Kush, Chem 91, Domnesia, Sour Tsunami, Sour Tsunami_x_CK, Tibor_1_2016, 80 E-1, 80 E-2, 80 E-3, Harlox, Saint Jack, Herijuana, Mothers Milk_5, Black Beauty, Sour Diesel, JL_1, JL_2, JL_3, JL_4, JL 5, JL 6, JL_father, BBCC_x_JL_father, JL mother, JL_mother_p, IdaliaFT_1, Fedora17_6_1, Carmal_1_2016, CS_1_2016, EICam_1_2016, C3/USO-1, Carmagnola_3, and Merino_S_1.

In any of the methods provided herein, in certain embodiments, the methods further include, based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for in-breeding or out-crossing, or for cultivating as a crop. In certain embodiments, the plant cultivar is selected for its lineage that is assigned, and/or a medicinal use that is assigned based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof. In embodiments, the plant cultivar is selected for resistance to an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof. In certain embodiments, the plant cultivar is selected for having an affinity towards an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof. In embodiments, the organism or situation is selected from among insects, pests, mold, pesticides and other chemicals, mildew, fungi, bacteria, viruses and other pathogens, an environmental condition, such as climate or soil conditions, or a geographic location. In certain embodiments, the plant cultivar is selected for root-specific, stem-specific, leaf-specific or flower-specific expression of a terpene synthase gene and/or paralog thereof, a terpene, a cannabinoid or a flavonoid based on identifying one or more terpene synthase genes, determining the expression profile of one or more terpene synthase genes, determining the production profile of one or more terpenes, determining the production profile of one or more cannabinoids, determining the production profile of one or more flavonoids or a combination thereof. Also provided herein are methods of breeding any of the plant cultivars selected according to the methods provided herein. Also provided herein are methods of cultivating a crop of any of the plant cultivars selected according to the methods provided herein. Also provided herein are methods of treatment comprising administering a plant cultivar selected according to the methods provided herein, or a portion thereof (e.g., seed, root, stem, flower) or an extract thereof (e.g., extracts from tissues of the plant cultivar) to a subject having a condition or disease in need of such treatment, whereby the condition or disease, or symptoms thereof, are ameliorated or reduced.

In certain embodiments of the methods provided herein, the methods include, based on identifying one or more terpene synthase genes and/or paralogs thereof, determining the expression profile of the one or more terpene synthase genes and/or paralogs thereof, determining the production profile of one or more terpenes, determining the production profile of one or more cannabinoids, determining the production profile of one or more flavonoids or a combination thereof, genetically modifying a plant cultivar whereby the expression of at least one terpene synthase gene and/or paralog thereof is inhibited or increased in the plant cultivar. In certain embodiments, the genetic modification increases the production of at least one terpene or decreases the production of at least one terpene in the plant cultivar. In embodiments, the plant cultivar is of a Cannabis cultivar. In certain aspects, the Cannabis cultivar is selected from among one or more of Type 1, Type 2, Type 3, Type 4 and Type 5 cultivars. In embodiments, one or more plant cultivars are of the family Rosidae.

In certain embodiments, the genetic modification increases the production of at least one cannabinoid or decreases the production of at least one cannabinoid in the plant cultivar. In embodiments, the genetic modification is for imparting a medicinal use. In embodiments, the genetic modification is for imparting resistance to an organism or situation. In certain embodiments, the genetic modification is for imparting affinity towards an organism or situation. In certain embodiments, the organism or situation is selected from among exposure to insects, pests, pesticides or other chemicals, mold, mildew, fungi, bacteria, viruses or other pathogens, an environmental condition, such as climate or soil conditions, or a geographic location. In embodiments, the genetic modification is for imparting root-specific, stem-specific, leaf-specific or flower-specific expression or inhibition of expression of a terpene synthase gene, a terpene, a cannabinoid or a flavonoid. The genetic modifications can be made by several methods known to those of skill in the art including, but not limited to, ZFN (Zinc Finger Nuclease), TALEN (Transcription Activator-Like Effector Nucleases), CRISPR-cas (cas9, cas12, cas13), Cre-Lox, MiRNA, SiRNA, ShRNA or a combination thereof.

In any of the methods provided herein, the unique subsequence of at least one terpene synthase gene or paralog thereof is outside the sequence encoding the active site of the terpene synthase gene or paralog thereof. In certain embodiments, the unique subsequence of at least one terpene synthase gene or paralog thereof is within the sequence encoding the active site of the terpene synthase gene or paralog thereof.

Also provided herein are methods of producing a daughter plant cultivar, that include:
analyzing two or more parent plant cultivars by the method provided herein;
based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting two parent plant cultivars for producing a desired daughter plant cultivar; and
inbreeding or outcrossing the parent plant cultivars to produce a daughter plant cultivar.

The term “daughter” cultivar is used interchangeably with “offspring” cultivar, i.e., the product of breeding parent cultivars.

In certain embodiments, the daughter plant cultivar produced has increased expression of at least one terpene synthase gene and/or paralog thereof or decreased expression of at least one terpene synthase gene and/or paralog thereof compared to at least one of the parent plant cultivars. In embodiments, the daughter plant cultivar produced has increased production of at least one terpene or decreased production of at least one terpene compared to at least one of the parent plant cultivars. In certain embodiments, the daughter plant cultivar produced has increased production of at least one flavonoid or decreased production of at least one flavonoid compared to at least one of the parent plant cultivars.

In certain embodiments, the parent plant cultivars and the daughter plant cultivar are Cannabis cultivars. In aspects, the Cannabis cultivar is selected from among one or more of Type 1, Type 2, Type 3, Type 4 and Type 5 cultivars. In embodiments, one or more plant cultivars (parent or daughter) are of the family Rosidae.

In embodiments, the daughter plant cultivar produced has increased production of at least one cannabinoid or decreased production of at least one cannabinoid compared to at least one of the parent plant cultivars.

In embodiments, the daughter plant cultivar has a medicinal use that is reduced or absent in the parent plant cultivars. In certain embodiments, the daughter plant cultivar has increased resistance to an organism or situation, where the resistance is reduced or absent in the parent plant cultivars. In embodiments, the daughter plant cultivar has increased affinity towards an organism or situation, where the affinity is reduced or absent in the parent plant cultivars. In embodiments, the organism or situation is selected from among insects, pests, mold, pesticides and other chemicals, mildew, fungi, bacteria, viruses and other pathogens, an environmental condition, such as climate or a soil condition, or a geographic location.

In embodiments, the daughter plant cultivar has increased root-specific, stem-specific, leaf-specific or flower-specific expression or inhibition of expression of a terpene synthase gene, a terpene, a cannabinoid or a flavonoid compared to at least one of the parent plant cultivars.

Also provided herein is a method of cultivating a crop, the method including:
analyzing at least one plant cultivar by any of the methods provided herein;
based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for producing a desired crop; and
cultivating the crop.

Also provided herein is a method of treatment of a subject having a disease or condition, the method including:
analyzing at least one plant cultivar by any of the methods provided herein;
based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for a desired treatment of the disease or condition; and
administering the plant cultivar or a portion or extract thereof to the subject.

Also provided herein are methods of genetically modifying a plant cultivar, that include:
analyzing the plant cultivar by the methods provided herein; and based on the analysis, altering at least one unique subsequence of at least one terpene synthase gene or a paralog thereof, whereby expression of the at least one terpene synthase gene or paralog thereof is increased or decreased compared to in the absence of the alteration and whereby one or more of the terpene synthase gene and/or paralog thereof expression profile, the terpene production profile, the cannabinoid production profile or the flavonoid production profile of the plant is modified. In certain embodiments, the genetically modified plant cultivar has increased expression of at least one terpene synthase gene or a paralog thereof or decreased expression of at least one terpene synthase gene or a paralog thereof, compared to the unmodified plant cultivar. In certain embodiments, the genetically modified plant cultivar has increased production of at least one terpene or decreased production of at least one terpene compared to the unmodified plant cultivar. In embodiments, the plant cultivar that is genetically modified is of a Cannabis cultivar. In aspects, the Cannabis cultivar is selected from among one or more of Type 1, Type 2, Type 3, Type 4 and Type 5 cultivars. In embodiments, one or more plant cultivars are of the family Rosidae. In embodiments, the genetically modified plant cultivar has increased production of at least one cannabinoid or decreased production of at least one cannabinoid compared to the unmodified plant cultivar.

In certain embodiments, the genetically modified plant cultivar has a medicinal use that is relatively reduced or absent in the unmodified plant cultivar. In embodiments, the genetically modified plant cultivar has increased resistance to an organism or situation, where the resistance is less than or absent in the unmodified plant cultivar. In embodiments, the genetically modified plant cultivar has increased affinity towards an organism or situation, where the affinity is reduced or absent in the unmodified plant cultivar. In embodiments, the organism or situation is selected from among exposure to insects, pests, mold, pesticides and other chemicals, mildew, fungi, bacteria, viruses and other pathogens, an environmental condition such as climate or soil conditions, or a geographic location. In embodiments, the genetically modified plant cultivar has increased root-specific, stem-specific, leaf-specific or flower-specific expression or inhibition of expression of a terpene synthase gene, a terpene, a cannabinoid or a flavonoid compared to the unmodified plant cultivar.

In certain embodiments, the genetic modification is by a method such as ZFN, TALEN, CRISPR-cas, Cre-Lox, MiRNA, SiRNA, ShRNA or a combination thereof. In embodiments, the expression of two or more terpene synthase genes and/or paralogs thereof is specifically increased or specifically inhibited.

Also provided herein is a method of analyzing a gene of a plant cultivar that belongs to a family of genes, wherein the gene comprises two or more exons, that includes:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique exon of the gene or a portion thereof, wherein the unique exon of the gene is different than the other exons of the gene and the unique exon of the gene is different than the exons of the other genes in the gene family;
(c) amplifying the mixture, thereby obtaining an amplified mixture; and
(d) analyzing the amplified mixture of (c), whereby the gene of the plant cultivar is identified and/or quantified in the amplified mixture.

Also provided herein is a method of analyzing a family of genes of a plant cultivar, wherein each gene of the family comprises two or more exons, that includes:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique exon of a gene or a portion thereof, wherein the unique exon of the gene is different than the other exons of the gene and the unique exon of the gene is different than the exons of the other genes in the family of genes;
(c) amplifying the mixture, thereby obtaining an amplified mixture; and
(d) analyzing the amplified mixture of (c), whereby at least one gene of the family of genes is identified and/or quantified in the amplified mixture.

In certain embodiments, wherein the family of genes comprises terpene synthase genes and/or paralogs thereof.

Also provided herein is a solid support that includes a single-stranded polynucleotide species, wherein the single-stranded polynucleotide species specifically binds to a unique subsequence of a terpene synthase gene or a paralog thereof, wherein the unique subsequence of the terpene synthase gene or paralog thereof is different than the other subsequences of the terpene synthase gene or paralog thereof and the unique subsequence of the terpene synthase gene or paralog thereof is different than the subsequences of other terpene synthase genes and/or paralogs thereof. In certain embodiments, the single-stranded polynucleotide species specifically binds to a conserved region of the unique subsequence. In embodiments, the unique subsequence is an exon, an intron, a portion within an exon or a portion within an intron. In certain embodiments, the unique subsequence is an exon or a portion within an exon. In embodiments, the single-stranded polynucleotide species is selected from among SEQ ID NOS: 1-1284. In embodiments, the single-stranded polynucleotide species is selected from among those set forth in SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284.

In embodiments, the terpene synthase gene or paralog thereof is a monoterpene synthase gene or paralog thereof, a diterpene synthase gene or paralog thereof or a sesquiterpene synthase gene or paralog thereof. In certain embodiments, the single-stranded polynucleotide species specifically binds to a unique subsequence of a terpene synthase gene or a paralog thereof from a Cannabis cultivar. In aspects, the Cannabis cultivar is selected from among one or more of Type 1, Type 2, Type 3, Type 4 and Type 5 cultivars. In embodiments, one or more plant cultivars are of the family Rosidae. In embodiments, the solid supports provided herein can be selected from among a bead, column, capillary, disk, filter, dipstick, membrane, wafer, comb, pin or a chip. In embodiments, the solid supports are made from a material selected from among silicon, silica, glass, controlled-pore glass (CPG), nylon, Wang resin, Merrifield resin, Sephadex, Sepharose, cellulose, magnetic beads, Dynabeads, a metal, a metal surface, a plastic or polymer or combinations thereof.

In certain embodiments, the unique subsequence of at least one terpene synthase gene or paralog thereof is outside the sequence encoding the active site of the terpene synthase gene or paralog thereof. In embodiments, the unique subsequence of at least one terpene synthase gene or paralog thereof is within the sequence encoding the active site of the terpene synthase gene or paralog thereof.

Also provided herein is a collection of the solid supports provided herein, wherein:
(a) each solid support in the collection contains a single-stranded polynucleotide species, wherein the single-stranded polynucleotide species specifically binds to a unique subsequence of a terpene synthase gene or a paralog thereof, wherein the unique subsequence of the terpene synthase gene or paralog thereof is different than the other subsequences of the terpene synthase gene or paralog thereof and the unique subsequence of the terpene synthase gene or paralog thereof is different than the subsequences of other terpene synthase genes and/or paralogs thereof; and
(b) the single-stranded polynucleotide species of each solid support in the collection is different than the single-stranded polynucleotide species of the other solid supports in the collection.

In certain embodiments, each single-stranded polynucleotide species in the collection specifically binds to a unique subsequence of the same terpene synthase gene or paralog thereof. In embodiments, each single-stranded polynucleotide species specifically binds to a unique subsequence of a terpene synthase gene or a paralog thereof that is different than the terpene synthase genes and/or paralogs thereof to which the other single-stranded polynucleotide species in the collection bind. In certain embodiments, the collection includes at least two single-stranded polynucleotide species that specifically bind to unique subsequences of the same terpene synthase gene and/or paralog thereof or at least two single-stranded polynucleotide species that specifically bind to unique subsequences of two different terpene synthase genes and/or paralogs thereof. In embodiments, each of the single-stranded polynucleotide species specifically binds to a conserved region of the unique subsequence. In certain embodiments, the unique subsequence is an exon, an intron, a portion within an exon or a portion within an intron. In embodiments, the subsequence is an exon or a portion within an exon. In certain embodiments, the single-stranded polynucleotide species are selected from among SEQ ID NOS: 1-1284. In embodiments, the single-stranded polynucleotide species are selected from among those set forth in SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284.

In certain embodiments, the terpene synthase genes and/or paralogs thereof are monoterpene synthase genes and/or paralogs thereof, diterpene synthase genes and/or paralogs thereof, sesquiterpene synthase genes and/or paralogs thereof, or any combination thereof. In certain embodiments of the collections provided herein, all or a portion of the single-stranded polynucleotide species specifically binds to a unique subsequence of a terpene synthase gene or a paralog thereof from a Cannabis cultivar. In aspects, the Cannabis cultivar is selected from among one or more of Type 1, Type 2, Type 3, Type 4 and Type 5 cultivars. In embodiments, one or more plant cultivars are of the family Rosidae.

In certain embodiments of the collections of solid supports provided herein, the solid supports are arranged in an array. In certain embodiments, the array is on a chip.

Also provided herein is a method of analyzing the terpene synthase gene profile of a plant cultivar, which includes:
(a) obtaining a nucleic acid sample from the plant cultivar;
(b) contacting the nucleic acid sample with the solid support collections provided herein, thereby preparing a mixture, wherein each single-stranded polynucleotide species of the collection can specifically bind to a unique subsequence of a terpene synthase gene or a paralog thereof, wherein the unique subsequence of the terpene synthase gene or a paralog thereof is different than the other subsequences of the terpene synthase gene or paralog thereof and the unique subsequence of the terpene synthase gene is different than the subsequences of other terpene synthase genes and/or paralogs thereof; an
(c) subjecting the mixture to conditions that facilitate specific binding of each single-stranded polynucleotide species of the collection to its corresponding unique subsequence of a terpene synthase gene or paralog thereof, when the unique subsequence is present in the nucleic acid sample, thereby obtaining a collection comprising bound single-stranded polynucleotide species; and
(d) analyzing the collection comprising bound single-stranded polynucleotide species of (c), whereby at least one terpene synthase gene or a paralog thereof is identified and/or quantified, and the terpene synthase gene profile of the plant cultivar is determined. In certain embodiments, each single-stranded polynucleotide species of the collection binds to a conserved region of its corresponding unique subsequence. In embodiments, the unique subsequence is an exon, an intron, a portion within an exon or a portion within an intron. In embodiments, the subsequence is an exon or a portion within an exon.

In certain embodiments, the identification in (d) is by a signal that is generated when a single-stranded polynucleotide species of the collection binds to its corresponding unique subsequence. In embodiments, the signal is an electrical signal, an electronic signal, a signal from an optical label, such as a chromophore, a dye, or a fluorescent label, or from a radiolabel. In certain embodiments, the single-stranded polynucleotide species are selected from among SEQ ID NOS: 1-1284. In embodiments, the single-stranded polynucleotide species are selected from among those set forth in SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284.

In embodiments, the terpene synthase genes and/or paralogs thereof that are identified and/or quantified in the terpene synthase gene profile are monoterpene synthase genes and/or paralogs thereof, diterpene synthase genes and/or paralogs thereof, sesquiterpene synthase genes and/or paralogs thereof or any combination thereof. In certain embodiments, the terpene synthase gene profile that is obtained, the terpene synthase gene and/or paralog expression profile and/or the terpene production profile of the plant cultivar is determined. In embodiments, the terpene synthase gene expression profile and/or the terpene production profile is of the root, flower, stem, leaf or any combination thereof.

In certain embodiments, based on the terpene synthase gene profile that is obtained and/or based on the terpene synthase gene expression profile that is determined and/or based on the terpene production profile that is determined, a lineage of the plant cultivar is assigned. In embodiments, based on the terpene synthase gene profile that is obtained and/or based on the terpene synthase gene expression profile that is determined and/or based on the terpene production profile that is determined, a medicinal use of the plant cultivar is assigned. In certain embodiments, based on the terpene synthase gene profile that is obtained and/or based on the terpene synthase gene expression profile that is determined and/or based on the terpene production profile that is determined, the plant cultivar is identified as resistant to an organism or situation, or having an affinity towards or favoring an organism or situation. In embodiments, the organism or situation is selected from among insects, pests, pesticides and other chemicals, mold, mildew, fungi, bacteria, viruses and other pathogens, an environmental condition, such as climate or soil conditions, or a geographic location. In certain embodiments, a plurality of plant cultivars are analyzed. In embodiments, the plant cultivars are of the same species. In embodiments, the plurality of plant cultivars are further classified based on lineage. In certain embodiments, the plurality of plant cultivars are further classified based on medicinal use.

In embodiments, one or more plant cultivars are of the family Rosidae. In embodiments, one or more of the plant cultivars is/are a Cannabis cultivar. In aspects, the Cannabis cultivar is selected from among one or more of Type 1, Type 2, Type 3, Type 4 and Type 5 cultivars. In certain embodiments, the monoterpene synthase gene and/or paralog profile of the Cannabis plant cultivar is obtained and, based on the monoterpene synthase gene and/or paralog profile obtained and/or the expression profile of the identified and/or quantified monoterpene synthase genes and/or paralogs thereof, the terpene production profile, the cannabinoid production profile, the flavonoid production profile, or the combination of two or more of the terpene production profile, the cannabinoid production profile and the flavonoid production profile of the Cannabis plant cultivar is determined. In certain embodiments, based on the monoterpene synthase gene and/or paralog profile obtained, the expression profile of the identified and/or quantified monoterpene synthase genes and/or paralogs thereof, the cannabinoid production profile, the flavonoid production profile, or the cannabinoid production profile and the flavonoid production profile that is determined, a lineage of the Cannabis plant cultivar is assigned. In certain embodiments, based on the monoterpene synthase gene and/or paralog profile obtained, the expression profile of the identified and/or quantified monoterpene synthase genes and/or paralogs thereof, the cannabinoid production profile, the flavonoid production profile, or the cannabinoid production profile and the flavonoid production profile that is determined, a medicinal use of the Cannabis plant cultivar is assigned.

In embodiments, a plurality of Cannabis plant cultivars are analyzed. In certain embodiments, the plurality of Cannabis plant cultivars are further classified based on lineage. In certain embodiments, the plurality of Cannabis plant cultivars are further classified based on medicinal use.

In certain embodiments, any of the plants assigned or classified according to a desired property, such as lineage or a medicinal use or as resistant to or favoring an organism or condition, can further be selected and used in methods provided herein, such as methods of breeding, methods of cultivating a crop, methods of treatment, or methods of genetically modifying a plant cultivar as provided herein. In certain embodiments, at least one plant cultivar that is analyzed produces one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In certain embodiments, a terpene production profile is determined for one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin. In certain embodiments, at least one plant cultivar that is analyzed expresses one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPSS, TPSS, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, the one or more terpene synthases are selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

In certain embodiments, the method further includes, based on obtaining the terpene synthase gene and/or paralog profile, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for in-breeding or out-crossing. In embodiments, the plant cultivar is selected for its lineage that is assigned based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof. In certain embodiments, the plant cultivar is selected for a medicinal use that is assigned based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof. In embodiments, the plant cultivar is selected for resistance to an organism or situation that is identified based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof. In certain embodiments, the plant cultivar is selected for having an affinity towards an organism or situation that is identified based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof. In embodiments, the organism or situation is selected from among insects, pests, mold, mildew, fungi, bacteria, an environmental condition or a geographic location.

In certain embodiments, the plant cultivar is selected for root-specific, stem-specific, leaf-specific or flower-specific expression of a terpene synthase, a terpene, a cannabinoid or a flavonoid based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof. In certain embodiments, based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof, a plant cultivar is selected for genetic modification whereby the expression of at least one terpene synthase gene is inhibited or increased in the plant cultivar. In certain embodiments, the genetic modification increases the production of at least one terpene or decreases the production of at least one terpene in the plant cultivar. In embodiments, the plant cultivar is of a Cannabis cultivar.

In certain embodiments, the genetic modification increases the production of at least one cannabinoid or decreases the production of at least one cannabinoid in the plant cultivar. In embodiments, the genetic modification is for imparting a medicinal use. In certain embodiments, the genetic modification is for imparting resistance to an organism or situation. In embodiments, the genetic modification is for imparting affinity towards an organism or situation. In certain embodiments, the organism or situation is selected from among insects, pests, pesticides and other chemicals, mold, mildew, fungi, bacteria, viruses and other pathogens an environmental condition or a geographic location.

In certain embodiments, the genetic modification is for imparting root-specific, stem-specific, leaf-specific or flower-specific expression or inhibition of expression of a terpene synthase gene, a terpene, a cannabinoid or a flavonoid. In embodiments, the genetic modification is by a method that includes ZFN (Zinc Finger Nuclease), TALEN (Transcription Activator-Like Effector Nucleases), CRISPR-cas, Cre-Lox, MiRNA, SiRNA, ShRNA or a combination thereof. In certain embodiments, the unique subsequence of at least one terpene synthase gene is outside the sequence encoding the active site of the terpene synthase gene.

The plant cultivars analyzed and/or genetically modified according to the methods provided herein can be used in methods of breeding, of cultivating crops, of treatment and other uses as provided herein. In certain embodiments, when the plant cultivars are genetically modified, they can be screened for the existence of the genetic modification using any of the solid supports or collections of solid supports according to any of the methods provided herein. In embodiments, the existence of a mutation in a plant cultivar can be detected using any of the solid supports or collections of solid supports according to any of the methods provided herein.

Also provided herein are kits that include:
one or more single-stranded polynucleotide species, wherein each single-stranded polynucleotide species specifically binds to a unique subsequence of a terpene synthase gene, wherein the unique subsequence of the terpene synthase gene is different than the other subsequences of the terpene synthase gene and the unique subsequence of the terpene synthase gene is different than the subsequences of other terpene synthase genes; and
instructions for use in obtaining a terpene synthase gene profile of a plant cultivar and/or for detecting a mutation in one or terpene synthase genes of a plant cultivar. In certain embodiments, the single-stranded polynucleotide species specifically binds to a conserved region of the unique subsequence. In embodiments, the genes include paralogs. In certain embodiments, the unique subsequence is an exon, an intron, a portion within an exon or a portion within an intron. In embodiments, the unique subsequence is an exon or a portion within an exon.

In certain embodiments of the kits provided herein, the one or more single-stranded polynucleotide species are selected from among SEQ ID NOS: 1-1284. In embodiments, the one or more single-stranded polynucleotide species are selected from among those set forth in SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284.

In embodiments, the terpene synthase gene is a monoterpene synthase gene or a paralog thereof, a diterpene synthase gene or a paralog thereof, or a sesquiterpene synthase gene or a paralog thereof. In certain embodiments, each single-stranded polynucleotide species specifically binds to a unique subsequence of a terpene synthase gene from a Cannabis cultivar.

In certain embodiments, the kits provided herein further include a label for detecting the specific binding of each single-stranded polynucleotide species to a corresponding unique subsequence of a terpene synthase. In certain embodiments, if more than one single-stranded polynucleotide species is present, the single-stranded polynucleotide species bind to different unique subsequences of the same terpene synthase gene, to different unique subsequences of different terpene synthase genes, or to different unique subsequences of the same terpene synthase gene and to different unique subsequences of different terpene synthase genes. In certain embodiments, the unique subsequence of at least one terpene synthase gene is outside the sequence encoding the active site of the terpene synthase gene. In certain embodiments, the unique subsequence of at least one terpene synthase gene is within the sequence encoding the active site of the terpene synthase gene.

Also provided herein are kits that include:
one or more polynucleotide primer pairs, wherein each polynucleotide primer pair hybridizes to a unique subsequence of a terpene synthase gene, wherein the unique subsequence of the terpene synthase gene is different than the other subsequences of the terpene synthase gene and the unique subsequence of the terpene synthase gene is different than the subsequences of other terpene synthase genes; and
instructions for use in analyzing the nucleic acid of a plant cultivar.

In certain embodiments, each of the primers of a polynucleotide primer pair hybridizes to a conserved region of the subsequence and the hybridized polynucleotide primer pair flanks a variable region of the subsequence. In embodiments, the unique subsequence is an exon, an intron, a portion within an exon or a portion within an intron. In certain embodiments, the unique subsequence is an exon or a portion within an exon. In certain embodiments, each primer pair is selected from among SEQ ID NOS: 1-1284. In embodiments, each primer pair is selected from among SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284.

In embodiments, the terpene synthase gene is a monoterpene synthase gene, a diterpene synthase paralog or a sesquiterpene synthase paralog. In certain embodiments, each polynucleotide primer pair specifically binds to a unique subsequence of a terpene synthase paralog from a Cannabis cultivar. In certain embodiments, if more than one polynucleotide primer pair is present, each polynucleotide primer pair binds to different unique subsequences of the same terpene synthase paralog, to different unique subsequences of different terpene synthase paralogs, or to different unique subsequences of the same terpene synthase paralog and to different unique subsequences of different terpene synthase paralogs.

In embodiments, the kits provided herein further include reagents for amplification of nucleic acid from a plant cultivar. In embodiments, the unique subsequence of at least one terpene synthase paralog is outside the sequence encoding the active site of the terpene synthase paralog. In embodiments, the unique subsequence of at least one terpene synthase paralog is within the sequence encoding the active site of the terpene synthase paralog.

Also provided herein are methods of preparing primers for uniquely amplifying a gene or a paralog thereof, which includes:
(a) identifying, within at least one exon of the gene or paralog thereof, a first sequence and a second sequence that are conserved for that gene or paralog thereof, wherein the first conserved sequence and the second conserved sequence flank a sequence that is not conserved; and
(b) preparing a pair of primers, wherein:
   (i) one primer hybridizes to the first conserved sequence and one primer hybridizes to the second conserved sequence;
   (ii) each primer, when hybridized to a sequence of the gene or paralog thereof that is not its target conserved sequence, has at least 5 mismatches with the sequence; and
   (iii) each primer, when hybridized to a sequence of the gene or paralog thereof that is not its target conserved sequence, has at least 3 mismatches with the sequence within 5 bases from the 3′-end of the primer.

In certain embodiments, the melting temperature of each primer hybridized to its target conserved sequence is between about 57° C. to about 63° C. In embodiments, the difference between the melting temperatures of each primer of the primer pair hybridized to its target sequence is 3° C. or less.

In certain embodiments, for at least one exon of the gene or paralog thereof, more than one primer pair is prepared, wherein each primer pair amplifies a difference sequence within the exon of the gene or paralog thereof. In embodiments, more than one primer pair is prepared and at least two primer pairs amplify sequences within two different exons of the gene or paralog thereof. In certain embodiments, the gene or paralog thereof is of a terpene synthase gene.

In certain embodiments of any of the methods provided herein, the size of the product (amplicon) that is amplified by any of the pairs of primers provided herein, including primers prepared by any of the methods provided herein, is less than 300 base pairs, such as between about 30 base pairs to about 295 base pairs, or between about 40 base pairs to about 200 base pairs, or between about 50 base pairs to about 150 base pairs. For example, the size of the product (amplicon) that is amplified by any of the pairs of primers provided herein, including primers prepared by any of the methods provided herein, can be, e.g., about 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290 or 295 base pairs, within +1- about 10% of each of the recited sizes of the amplicons.

Also provided herein are genetically modified plant cultivars produced by any of the methods provided herein. The genetically modified plant cultivars can, in certain embodiments, be screened for the presence of a genetic modification using the solid supports and collections of solid supports according to the methods provided herein. In embodiments, the genetically modified plant cultivars can be used in the breeding methods, the methods of cultivating crops and the methods of treatment provided herein. In embodiments, a genetically modified plant cultivar is a Cannabis cultivar.

Also provided herein are methods of identifying whether a plant cultivar contains a terpene synthase gene or a paralog thereof that has been genetically modified, which includes:
(a) obtaining a nucleic acid sample from the plant cultivar;
(b) contacting the nucleic acid sample with any of the solid supports provided herein, or any of the collections of solid supports provided herein, thereby preparing a mixture, wherein at least one single-stranded polynucleotide species of the solid support or the collection can specifically bind to at least one genetically modified unique subsequence of a terpene synthase gene or a paralog thereof in the nucleic acid sample, when the at least one genetically modified unique subsequence is present, wherein the at least one genetically modified unique subsequence of the terpene synthase gene or a paralog thereof is different than the other subsequences of the terpene synthase gene or paralog thereof and the unique subsequence of the terpene synthase gene is different than the subsequences of other terpene synthase genes and/or paralogs thereof; and
(c) subjecting the mixture to conditions that facilitate specific binding of the at least one single-stranded polynucleotide species of the solid support or the collection to its corresponding at least one genetically modified unique subsequence of a terpene synthase gene or paralog thereof in the nucleic acid sample, when the at least one genetically modified unique subsequence is present in the nucleic acid sample; and
(d) detecting at least one single-stranded polynucleotide species of the solid support or the collection as being specifically bound to its corresponding genetically modified unique subsequence of a terpene synthase gene or paralog thereof in the nucleic acid sample wherein, if the at least one genetically modified unique subsequence is detected as specifically bound to a solid support or a collection in (c), the plant cultivar is identified as comprising a genetically modified terpene synthase or paralog thereof.

In certain embodiments, the detecting in (d) is by a signal that is generated when a single-stranded polynucleotide species of the collection binds to its corresponding genetically modified unique subsequence. In embodiments, the signal is an electrical signal, an electronic signal, a signal from an optical label, such as a dye or intercalator or fluorescent label, or from a radiolabel.

In certain embodiments, the method further includes, if the plant cultivar is identified as containing a genetically modified terpene synthase or paralog thereof, determining the type of genetic modification. In embodiments, the type of genetic modification is selected from among deletions, insertions and substitutions. In certain embodiments, the genetic modification includes at least one substitution. In embodiments, the at least one substitution is in a unique subsequence that expresses the active site of the terpene synthase, or a portion thereof.

In embodiments, the plant cultivar is a genetically modified plant cultivar provided herein or obtained by the methods provided herein. In embodiments, one or more plant cultivars are of the family Rosidae. In certain embodiments, the plant cultivar is a Cannabis cultivar. In aspects, the Cannabis cultivar is selected from among one or more of Type 1, Type 2, Type 3, Type 4 and Type 5 cultivars. In certain embodiments, at least one genetically modified unique subsequence is an exon, or contains an exon.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale, and, in some instances, various aspects may be shown exaggerated or enlarged to facilitate an understanding of particular embodiments.

FIG. 1 shows the hybridization/melting characteristics of the primers listed in Table 2.

FIG. 2 is a heatmap showing the presence, absence, and copy number variation of TPS genes (within 95% sequence identity) across six Cannabis genome assemblies.

FIG. 3 depicts the results of a LAMP assay for detecting the terpene synthase csTPS37FN in nucleic acid from Cannabis plant samples.

FIG. 4 provides examples of terpene synthases that can be analyzed and applied in uses according to the methods and compositions provided herein, along with the Citations/Accession Numbers that describe the synthases.

FIG. 5 provides examples of nucleic acid sequences encoding terpene synthases that can be analyzed and applied in uses according to the methods and compositions provided herein.

FIG. 6 provides a table as described herein.

FIG. 7 provides tables as described herein.

### DETAILED DESCRIPTION

Terpenes

Terpenes are aromatic compounds that are a class of unsaturated compounds found in the essential oils of many plants. As used herein, the term “plant” or “plant cultivar” includes any and all plant species that produce terpenes, including for example, angiosperms, any species of woody, ornamental or decorative, crop or cereal, fruit or vegetable, fruit plant or vegetable plant, flower or tree, macroalga or microalga, phytoplankton and photosynthetic algae (e.g., green algae Chlamydomonas reinhardtii). A plant also refers to a unicellular plant (e.g. microalga) and a plurality of plant cells that are largely differentiated into a colony (e.g. volvox) or a structure that is present at any stage of a plant's development. Such structures include, but are not limited to, a fruit, a flower, a seed, a shoot, a stem, a leaf, a root, plant tissue sand the like. As used herein, the term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue can be in planta, in organ culture, tissue culture, or cell culture. Any of the foregoing plant cultivars, portions thereof or extracts thereof are contemplated for use, e.g., as plant samples, in the methods provided herein.

The term “strain” is used interchangeably herein with “cultivar” (cultivated variety), “plant cultivar” or “variety” and refers to a species of a family of plants, such as a species of a Cannabis plant. A cultivar generally has been cultivated for desirable characteristics, such as color, shape, smell, medicinal use, etc., that are maintained during propagation. Phrases such as “plurality of strains of a plant” or “plurality of cultivars of a plant” or equivalent phrases, as used herein, refers to multiple species of the same plant, e.g., multiple species of Cannabis plant cultivars such as Jamaican Lion, Purple Kush, CannaTsu, Finola, Valley Fire, Cherry Chem and the like. The terms “strain,” “cultivar,” (cultivated variety), “plant cultivar” or “variety” also can be used interchangeably herein with “chemovar,” such as when the plant species is characterized by its chemical or biological profile, such as one or more of a terpene synthase gene profile, a terpene synthase expression profile, a terpene profile, a flavonoid profile, a cannabinoid profile or any combination thereof. The term “profile,” as used herein, can refer to the type and/or abundance (level of expression, in the case of a gene such as a terpene synthase) of each analyte of the group that is profiled, e.g., each terpene in a group of terpenes of a plant cultivar that are profiled, or each terpene synthase in a group of terpene synthasess of a plant cultivar that are profiled.

The molecular structures of terpenes consist of five carbon isoprene units. Mono terpenes contain 2 isoprene units, sesquiterpenes contain 3 isoprene units, and diterpenes contain 4 isoprene units.

These aromatic compounds create the characteristic scent of many plants, such as Cannabis, pine, and lavender, as well as fresh orange peel. The fragrance of most plants is due to a combination of terpenes. Terpenes play central roles in plant communication with the environment, including attracting beneficial organisms, repelling harmful ones, and communication between plants. In nature, these terpenes can protect the plants from animal grazing or infectious germs.

Terpenes also can offer health benefits to animals, including humans. Terpenes and essential oils have been studied over decades as remedies for a variety of medical conditions and have been found to have a wide range of biological and therapeutic properties. For example, terpenes are known to have antioxidant, anti-inflammatory, antibacterial, antiviral, anti-anxiety, antinociceptive, analgesic, antihypertensive, sedative, antidepressant, neuro protective and gastro protective properties. More recently, researchers have looked at the individual terpenes in essential oils, to understand which terpenoids might be contributing to their overall biological and medical properties. Terpenes in essential oils can either exert their individual effects in the oil or they can operate synergistically or agonistically with other oil constituents.

In Cannabis plants, such as C. sativa, more than 100 terpenes have been identified. Monoterpenes and sesquiterpenes are responsible for most of the odor and flavor properties of C. sativa, meaning that variation in terpene content is an important differentiator between cultivars. Therefore, there has long been interest from breeders in creating cultivars with particular terpene profiles. Further, there is a growing body of preliminary evidence that terpenes play a role in the various effects of C. sativa on humans, either directly or by modulating the effect of the cannabinoids, implying that medical C. sativa breeding likely will include terpene targets.

Given the important role of terpenes in plants, there is a need for methodologies to reliably identify plants that have desired terpene production profiles (used interchangeably herein with terpene profiles) for agricultural, industrial or medicinal use. The terms “terpene (production) profile,” “cannabinoid (production) profile,” “flavonoid (production) profile,” as used herein, refers to the types and amounts of terpenes, cannabinoids or flavonoids, respectively, in a plant cultivar, and can also include ratios of the relative abundance of two or more terpenes, cannabinoids or flavonoids, respectively.

Terpene synthases (TPSs) are the key enzymes responsible for the biosynthesis of terpenes. Provided herein are molecular markers that permit reliable identification and/or quantitation of the TPS gene profile of a plant cultivar, thereby permitting the identification and selection of plants for use in methods of genetic modification, methods of screening, and methods of use in breeding, crop cultivating, therapeutic methods and other methods as provided herein.

Terpene Synthases

The terpene synthase (TPS) family is a family of genes that encodes enzymes that use similar substrates and generate similar products but have diverged in different lineages to provide a wide variety of terpenes and mixtures of terpenes. Some estimates suggest that more than 25,000 terpene structures may exist in plants. Analysis of the several plant genomes that have been sequenced and annotated indicates that, with the exception of the moss Physcomitrella patens, which has a single functional TPS gene, the TPS gene family is a mid-size family, with gene numbers ranging from approximately 20 to 150 in sequenced plant genomes.

Isopentenyl diphosphate (IPP) is the common precursor of all terpenes. IPP is isomerized to give dimethylallyl pyrophosphate (DMAPP). DMAPP either serves as the substrate for hemiterpene biosynthesis or fuses with one IPP unit to form geranyl diphosphate (GPP). The condensation of one GPP molecule with one IPP molecule gives farnesyl diphosphate (FPP), and the condensation of one FPP molecule with one IPP molecule will give geranylgeranyl diphosphate (GGPP). GPP, FPP and GGPP are the precursors for monoterpenes, sesquiterpenes and diterpenes, respectively. While these prenyl diphosphates in the trans -configuration have been believed to be the ubiquitous natural substrates for terpene synthases, recent studies showed that two prenyl diphosphates in the cis-configuration, neryl diphosphate (NPP) and Z ,Z-FPP, are also the naturally occurring substrates of terpene synthases. Isoprene synthase, monoterpene synthases, sesquiterpene synthases, and diterpene synthases convert DMAPP, GPP (or NPP), FPP (or Z ,Z-FPP), and GGPP to isoprene, monoterpenes, sesquiterpenes and diterpenes, respectively.

Based on the reaction mechanism and products formed, plant TPSs can be classified into two groups: Class I and Class II. CPS is a representative of class II TPSs: it catalyzes the formation of CPP through protonation-induced cyclization of GGPP. However, most known plant TPSs are Class I TPSs. In the initial step of the enzymatic reactions catalyzed by Class I TPSs, the prenyl diphosphate is ionized and carbocation intermediates are formed.

A striking feature of class I TPS enzymes is that, because of the stochastic nature of bond rearrangements that follow the creation of the unusual carbocation intermediates, a single TPS enzyme using a single substrate often gives rise to multiple products. The central feature of this evolutionary plasticity is that change of just a single amino acid in the active site can lead to a different product profile. Most terpenes are secondary metabolites whose synthesis evolved in response to selection for increased fitness for some ecological niche. Consistent with the need (for terpene production profiles of a plant cultivar, e.g.) to be environmentally adaptive, the TPS family has evolved such that new TPS with differing product profiles can be derived from existing enzymes by changes to just a few amino acids.

Angiosperms, of which Cannabis is an example, tend to have moderately large families of these enzymes, with both divergent and convergent evolution taking place. Some of these TPS enzymes appear to be a result of recent duplications, i.e., they are paralogs of each other. Other TPS enzymes can be quite distant from each other: for example, there is a common “terpenoid synthase fold,” but sequence divergence across the family can be very high, just staying within the constraints of maintaining an overall fold and basic configuration of the active site.

Generally, the product profile of a given TPS enzyme cannot be determined from sequence similarity with other TPS enzymes. For example, two paralogous diterpene synthases in Norway spruce (Picea abies), isopimaradiene synthase and levopimaradiene/abietadiene synthase, although 91% identical at the amino acid level, differ in their terpene product profiles: one is a single-product enzyme, whereas the other is a multiproduct enzyme that forms completely different products. In addition, a one-amino acid mutation was found to switch the levopimaradiene/abietadiene synthase into producing isopimaradiene and sandaracopimaradiene and none of its normal products. Four mutations were sufficient to reciprocally reverse the product profiles for both of these paralogous enzymes, while maintaining catalytic efficiencies similar to the wild-type enzymes (Keeling et al., Proc. Natl. Acad. Sci. USA, 105(3):1085-1090 (2008).

Thus, given the widely differing terpene profiles of the TPS enzymes, even when there is a relatively high degree of overall sequence identity, there is a need to reliably identify the individual TPS genes that are present in the TPS gene profile of a plant cultivar. The methods provided herein are based on primer sets that amplify unique subsequences, such as exons or portions thereof, within each TPS gene, thereby providing a higher order differentiation that permits sensitive detection and/or quantification of each TPS gene in the plant cultivar, regardless of the overall sequence identity between the TPS genes.

Any terpene synthase, or combinations of terpene synthases, are contemplated for analysis and/or applications/uses according to the methods and compositions provided herein. In embodiments, terpene synthases contemplated herein for the compositions and methods of analyses/applications/uses include terpene synthases that produce, singly or in combinations of two or mor terpene synthases, one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, βcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

In certain embodiments, the one or more terpene synthases are selected from among those designated as TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPSS, TPSS, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, the one or more terpene synthases are selected from among those designated as TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

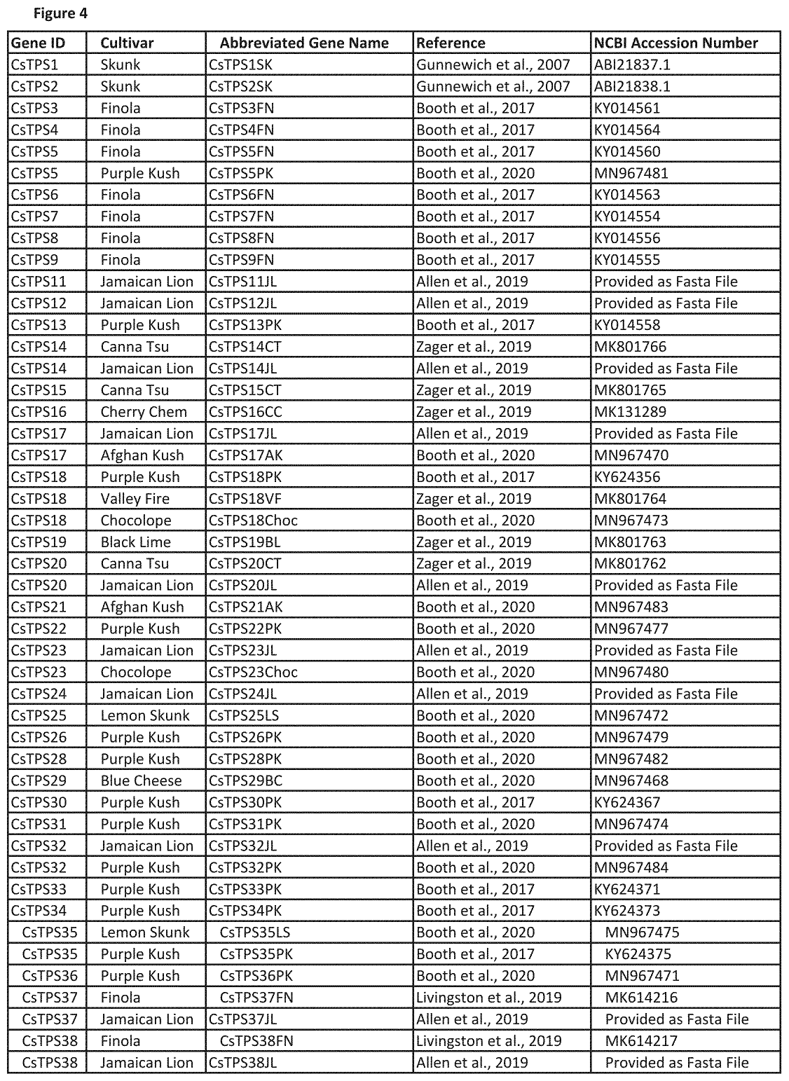

FIG. 4 provides examples of terpene synthases, along with citations that describe the terpene synthases and corresponding Accession Numbers, for certain compositions and methods of analyses/applications/uses as provided herein. The full citations corresponding to each of the abbreviated citations in FIG. 4 are as follows:
(1) “Günnewich et al. 2007”: Günnewich et al., Nat. Prod. Comm., 2(3):1934578x0700200301 (2007)
(2) “Booth et al. 2017”: Booth et al., PLoS ONE, 12(3): e0173911 (2017)
(3) “Allen et al. 2019”: Allen et al., Plant Physiol., 180(4):1877-1897 (2019)
(5) “Livingston et al. 2019”: Livingston et al., The Plant Jour., 101(1):37-56 (2019)
(6) “Booth et al. 2020”: Booth et al., Plant Physiol., 184(1):130-147 (2020)

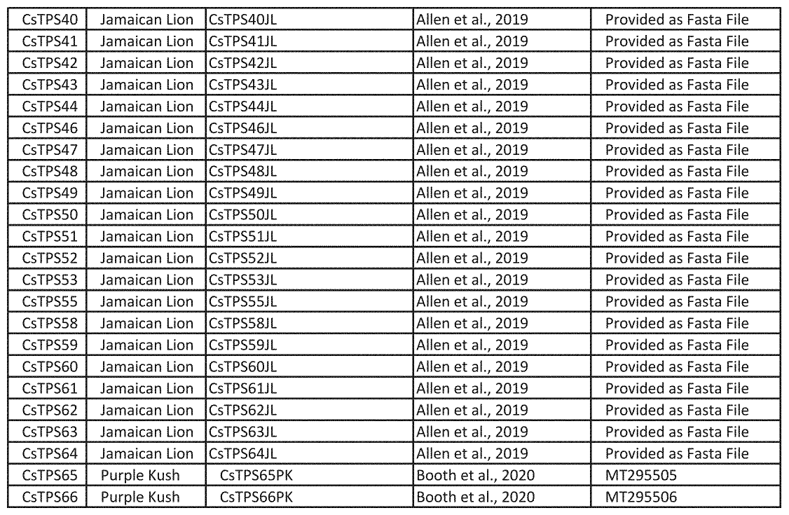

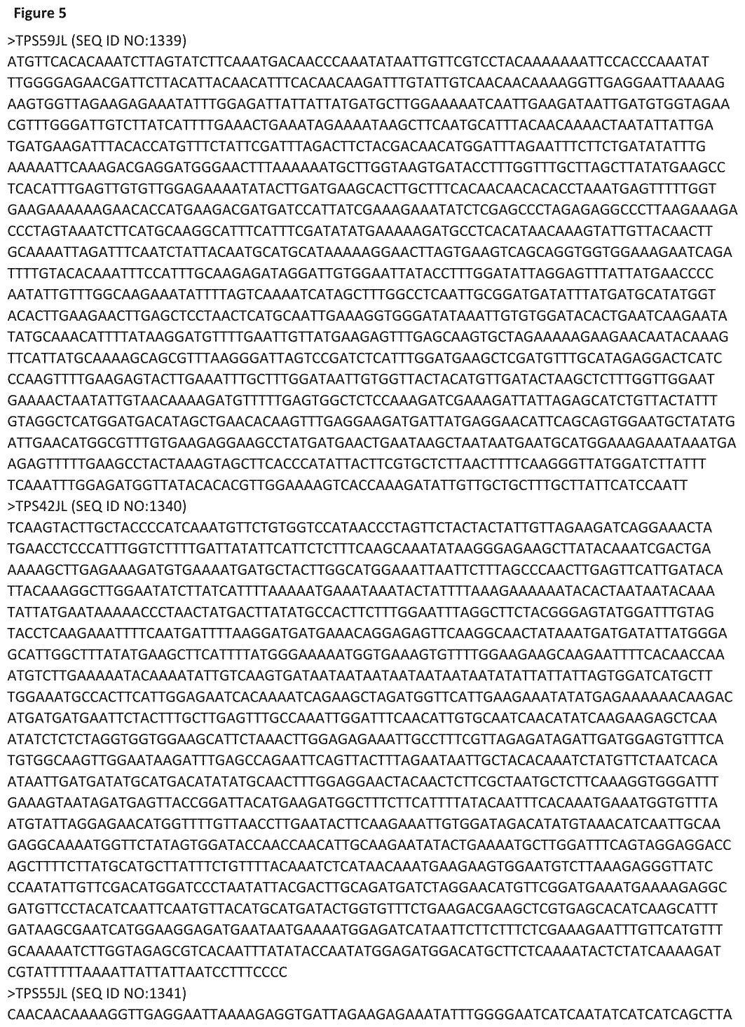

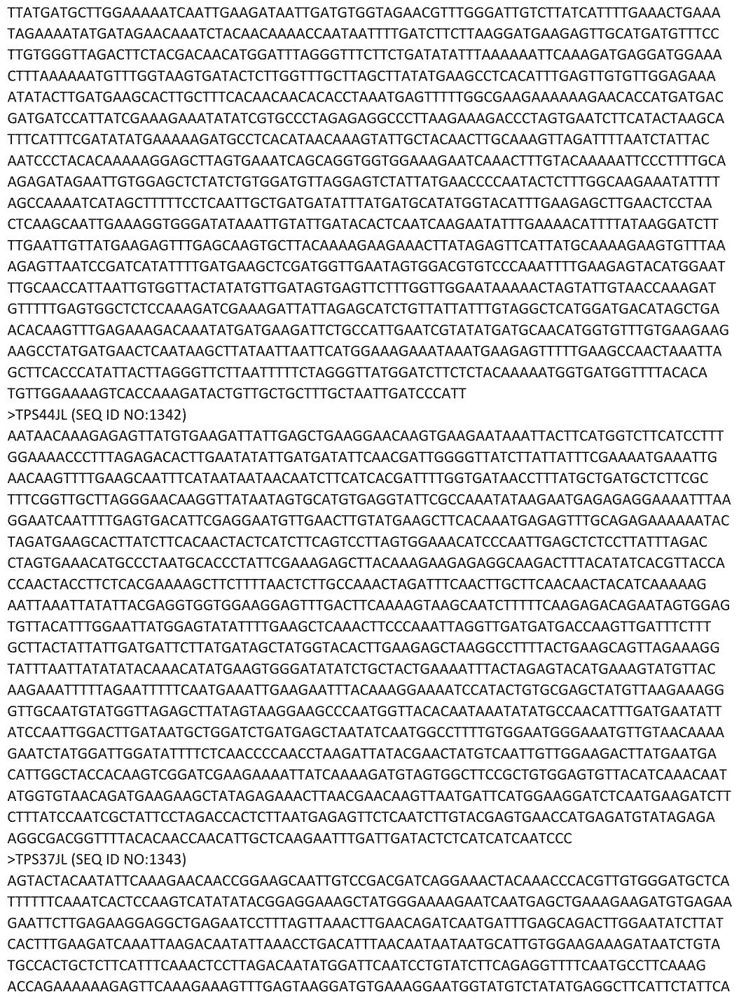

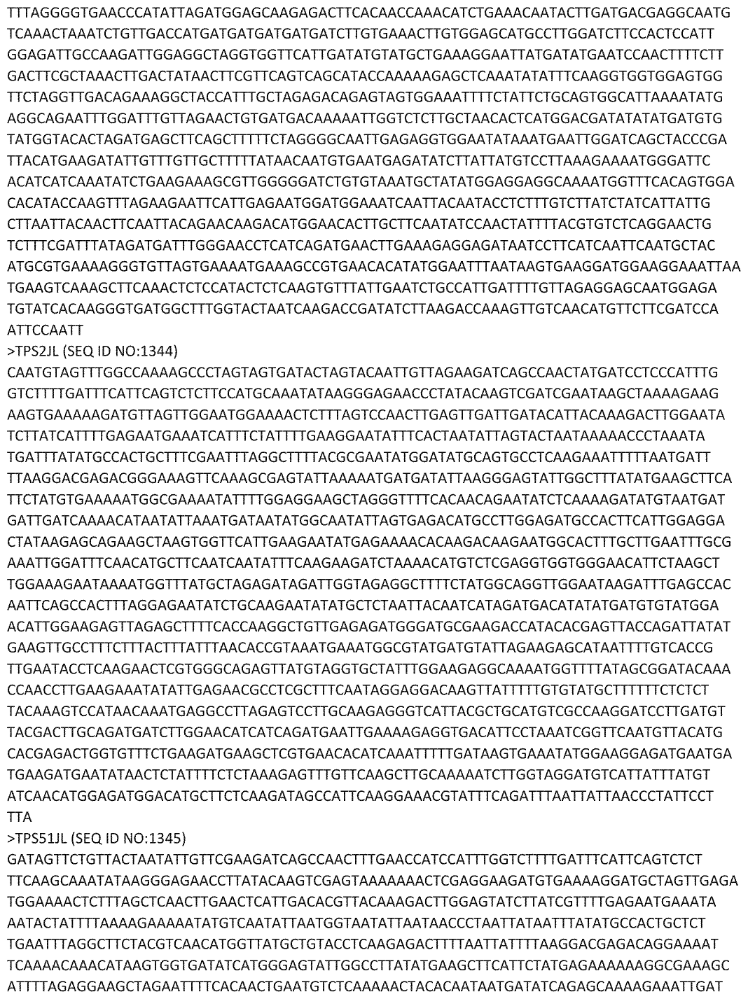

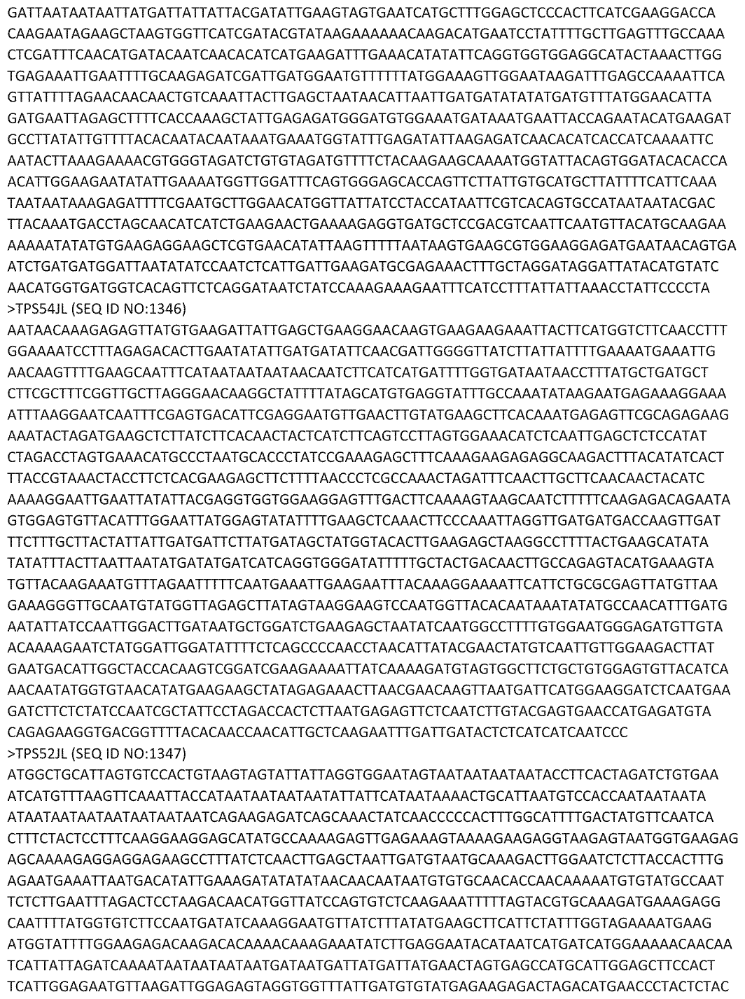

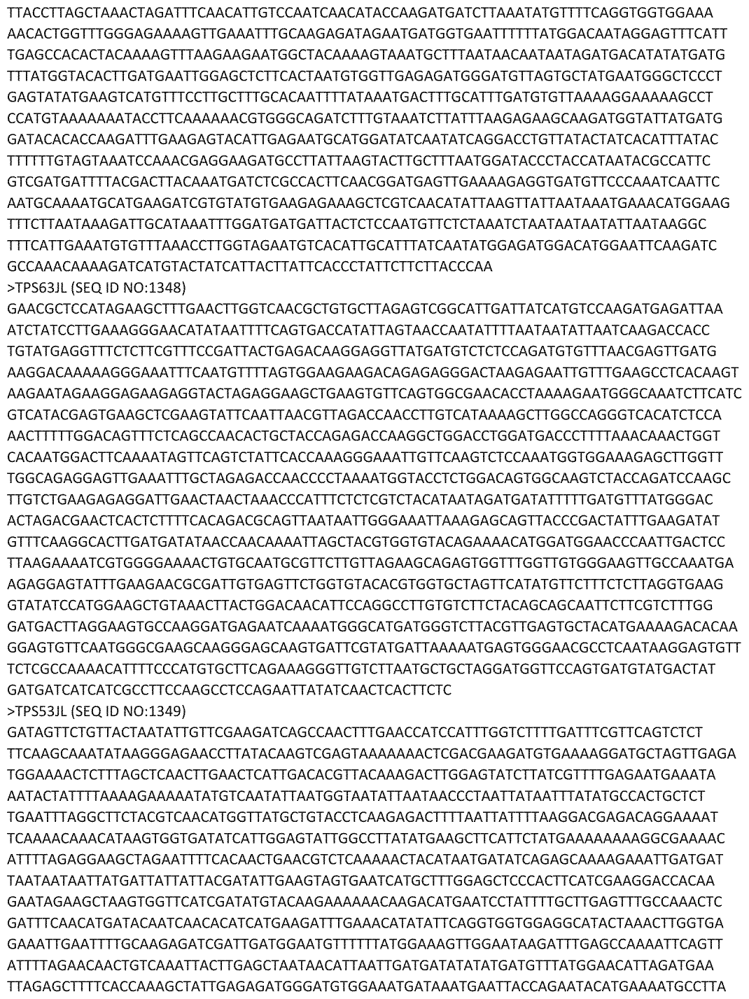

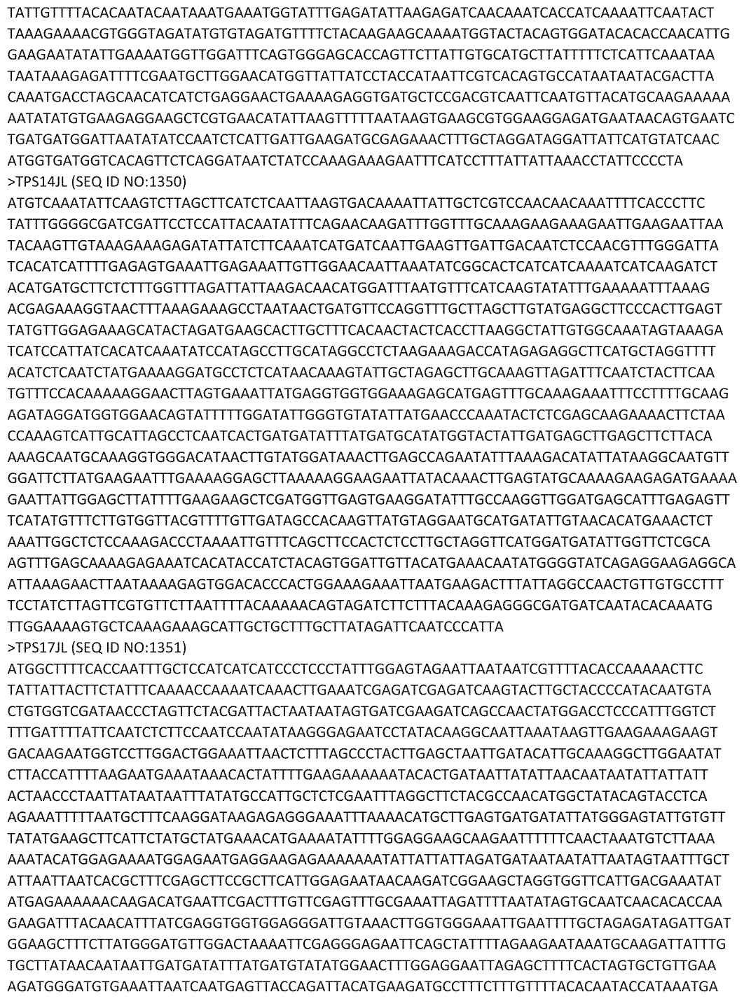

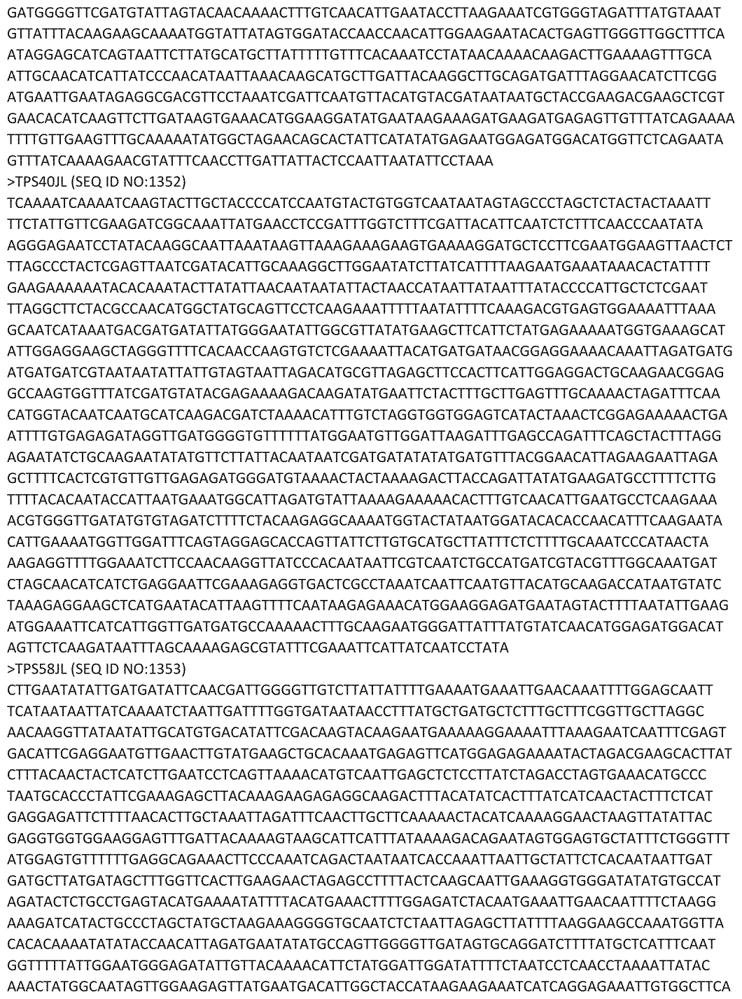

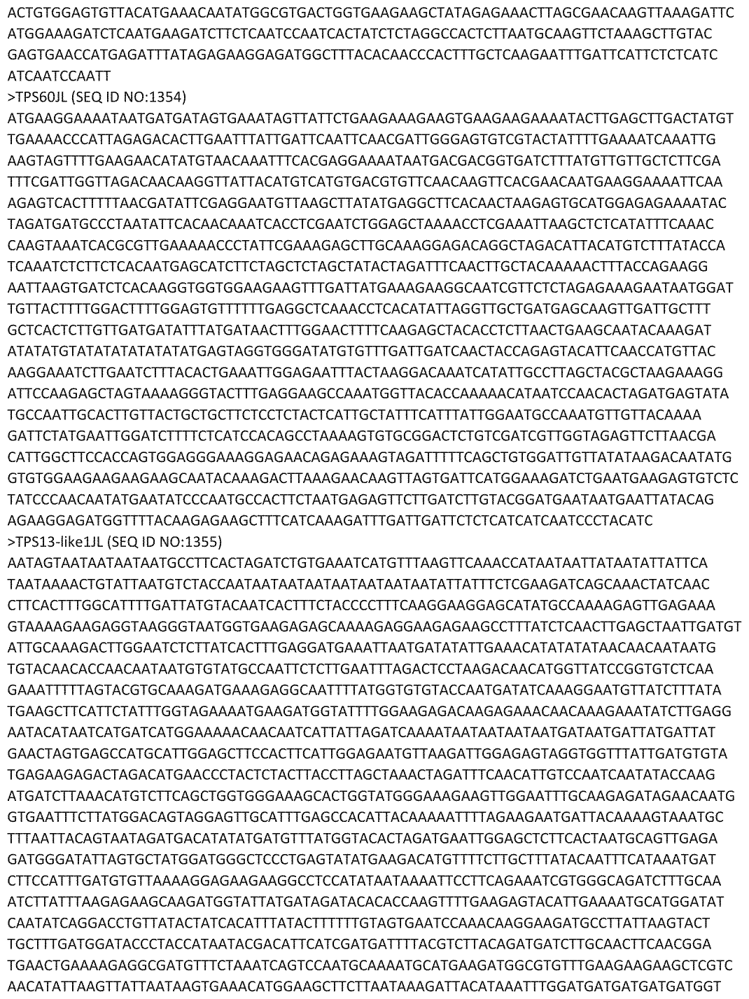

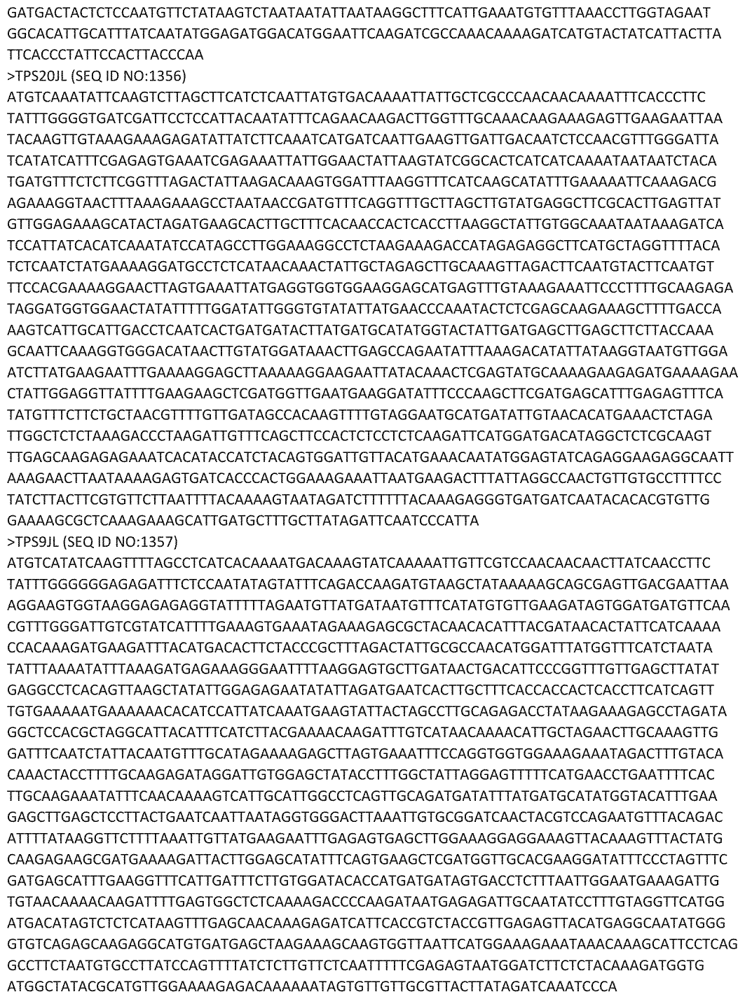

The contents of each of the above-cited documents are incorporated in their entirety by reference herein. Nucleic acid sequences encoding certain of the terpene synthases listed in FIG. 4 are provided in FIG. 5: these sequences are marked “Provided as Fasta File” in FIG. 4. FIG. 5 provides nucleic acid sequences (SEQ ID NOS:1339-1397) encoding examples of terpene synthases that can, in embodiments, be components of compositions and methods of analyses/applications/uses as provided herein.

It is understood that for any of the terpene synthases described herein for compositions and methods of analyses/applications/uses as provided herein, suitable conservative substitutions of amino acids are known to those of skill in this art and can generally be made without altering the biological activity of the resulting terpene synthases. Those of skill in the art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224). Such substitutions can be made, for example, in accordance with those set forth in TABLE 7 as follows:

TABLE 7

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible, including more than one conservative substitution and, in some instances, such conservative substitutions in active sites of the enzyme, such as a terpene synthase. The substaitutions can be determined empirically or in accord with known conservative substitutions.

Methods of Analyzing the TPS Gene Profile of a Plant Cultivar

Provided herein are methods and compositions for analyzing the TPS gene profile of a plant cultivar. The analyzing can include, for example, identifying and/or quantitating one or more TPS genes and/or paralogs thereof in a plant cultivar. The methods employ polymerase chain reaction (PCR) primers that are complementary to unique subsequences within each TPS gene that is in the genome of the plant cultivar, wherein hybridization of a subsequence of a TPS gene or paralog thereof to the primers uniquely identifies and/or quantitates the TPS gene or a paralog thereof. A unique subsequence of a TPS gene is a portion of the TPS gene that is different from other subsequences of the TPS gene and is different from subsequences of other TPS genes, thereby permitting identification of each TPS gene in the genome of the plant cultivar, such as a Cannabis genome. The subsequences can be an intron or a portion thereof, an exon or a portion thereof, or any region in-between that is identified as unique compared to other subsequences in the TPS gene and compared to the subsequences in other TPS genes. In embodiments, the subsequences to which the primers can be hybridized are exons, or portions thereof. In certain embodiments, more than one unique subsequence (e.g., exon) of a TPS gene can be analyzed, e.g., 2, 3, 4, 5, 6, 7, 8, 9, 10 or more unique subsequences (e.g., exons) of a TPS gene can be identified and/or quantitated, thereby increasing the accuracy of identifying a particular TPS gene in the genomic profile of a plant cultivar.

The primers provided herein can be used to amplify TPS genes and/or paralogs thereof prior to input in various common assays for variant identification, including high resolution melting (HRM), quantitative PCR (qPCR), loop-mediated isothermal amplification (LAMP), restriction endonuclease digestion, gel electrophoresis, and/or Sanger/Next-Generation sequencing. For each plant cultivar that is analyzed according to the methods provided herein, a barcode representing each TPS gene and/or paralog thereof that is identified and/or quantitated can be assigned, thereby providing an efficient way to visualize the TPS gene profile of a plant cultivar. The barcode for a given TPS gene can be based, for example, on the number and types of exons that are detected and/or quantified for that TPS gene—each detected exon can be assigned a number, and the total read of all detected exons can constitute a barcode.

Detection of TPS Genes or Paralogs Thereof

Provided herein are methods for analyzing nucleic acid from a plant sample. Also provided herein are methods for generating nucleic acid amplification products from a plant sample. Also provided herein are methods for preparing a nucleic acid mixture. A method herein can include contacting nucleic acid of a plant sample with a polynucleotide primer pair under amplification conditions. In embodiments, a method herein includes contacting nucleic acid of a plant sample with one or more polynucleotide primer pairs under amplification conditions. In some embodiments, a method herein comprises contacting nucleic acid of a plant sample with a plurality of polynucleotide primer pairs under amplification conditions. A plurality of primer pairs can include two or more polynucleotide primer pairs, three or more polynucleotide primer pairs, four or more polynucleotide primer pairs, five or more polynucleotide primer pairs, six or more polynucleotide primer pairs, seven or more polynucleotide primer pairs, eight or more polynucleotide primer pairs, nine or more polynucleotide primer pairs, or ten or more polynucleotide primer pairs. Each of the plurality of primer pairs can be used to analyze a sample in a separate reaction container, such as a well. Alternately, if the amplicons expected to be obtained using the plurality of primer pairs are expected to be of different sizes and/or are otherwise distinguishable (e.g., using labeled primers), a plurality of primers can be used to analyze the sample in a single reaction container.

In certain embodiments, a method includes generating one or more amplification products. Amplification products can be generated by any suitable amplification method described herein or known in the art (e.g., polymerase chain reaction (PCR)). Suitable amplification conditions can include any conditions that can generate an amplification product, when a target nucleic acid, such as a unique subsequence (e.g., exon) of a TPS gene, is contacted with primers that are capable of hybridizing to the target nucleic acid. In embodiments, a method includes generating a mixture (e.g., a mixture of two or more amplification product species). A mixture of two or more amplification product species can be generated when two or more primer pairs hybridize to different regions of a target nucleic acid. Such amplification product species can have different lengths and/or different nucleotide sequences, which can include overlapping and/or non-overlapping sequences.

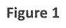

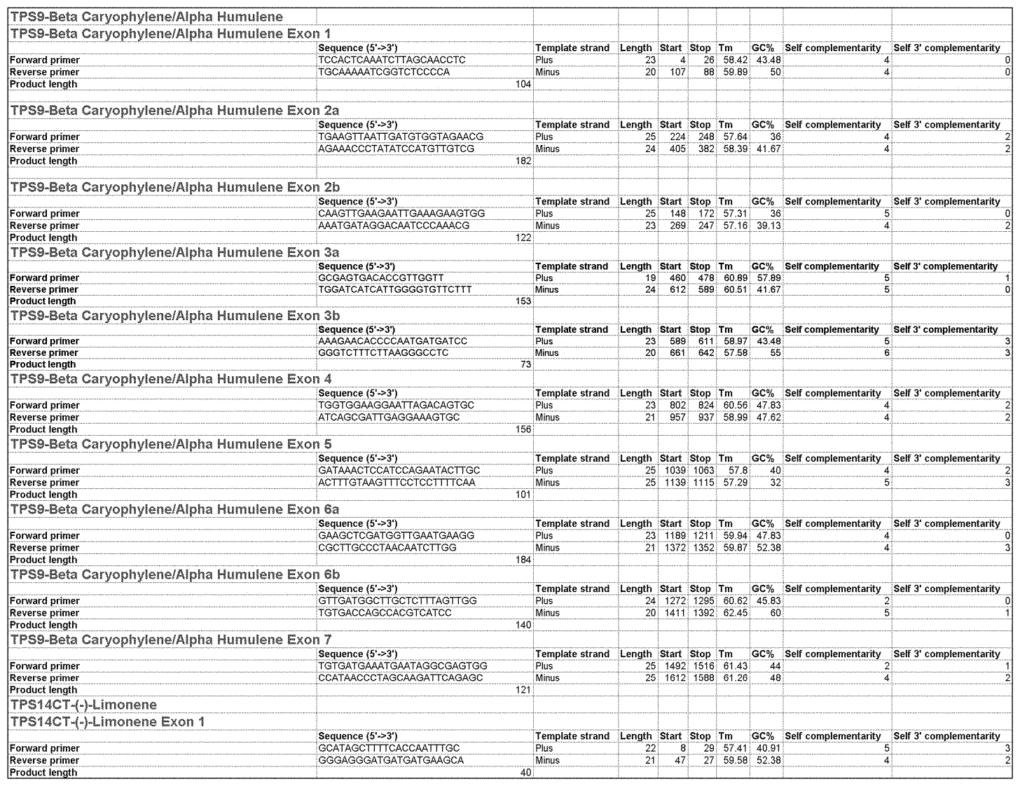

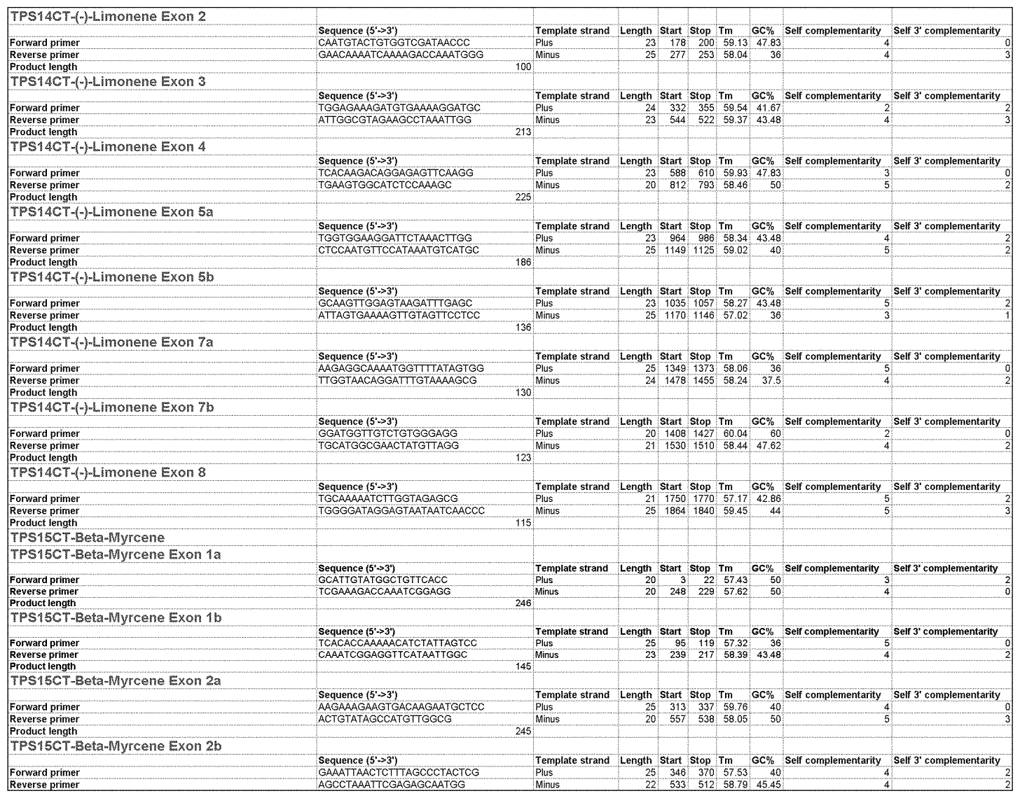

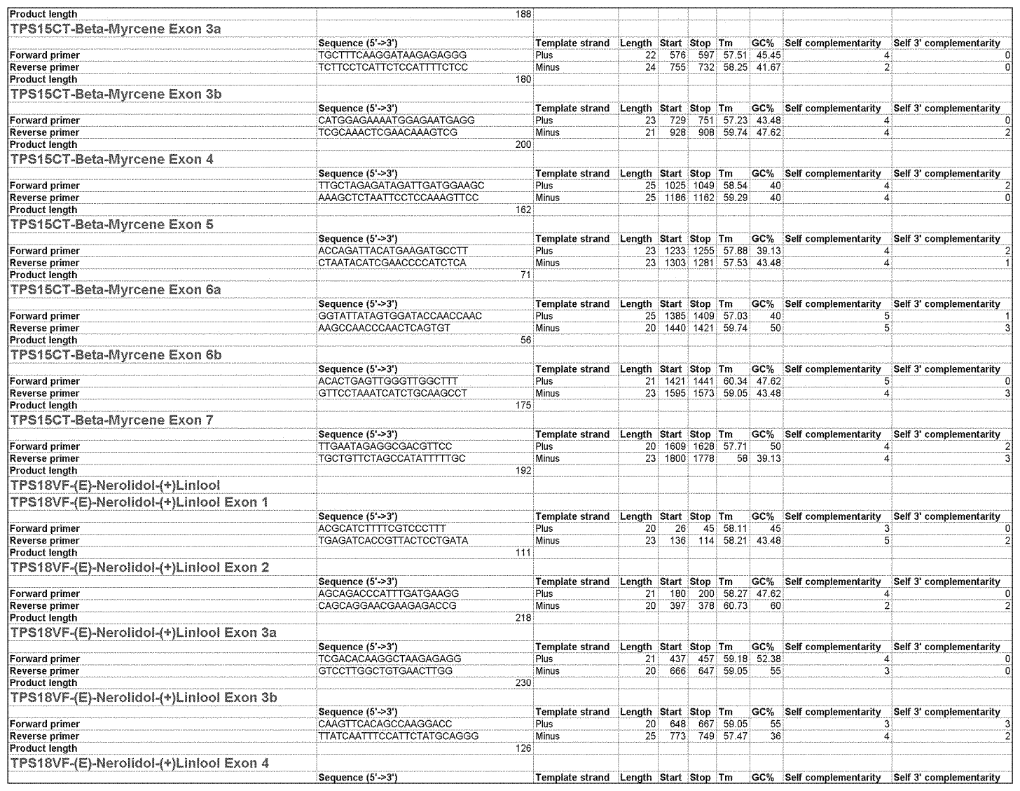

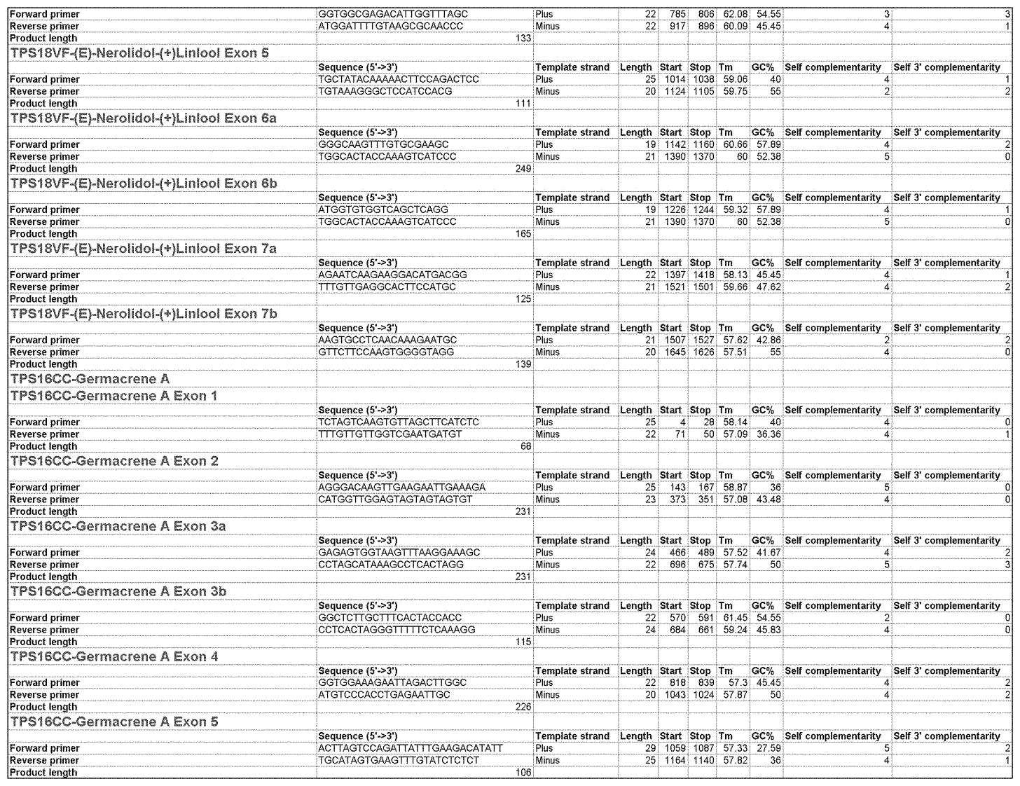

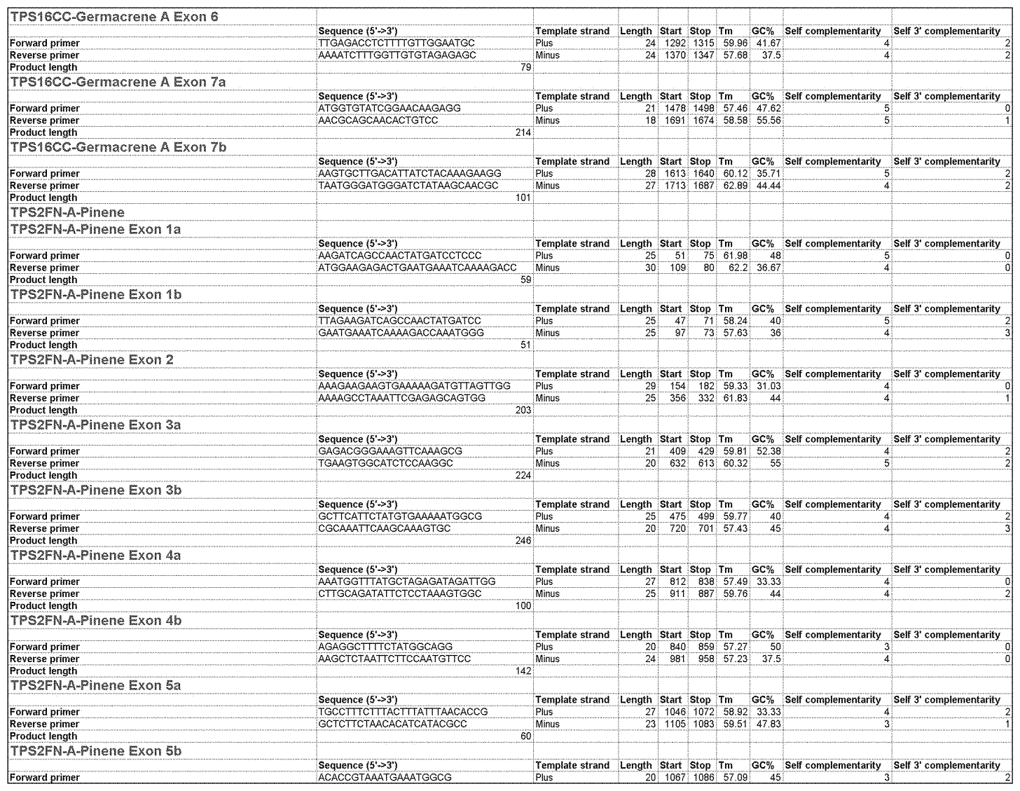

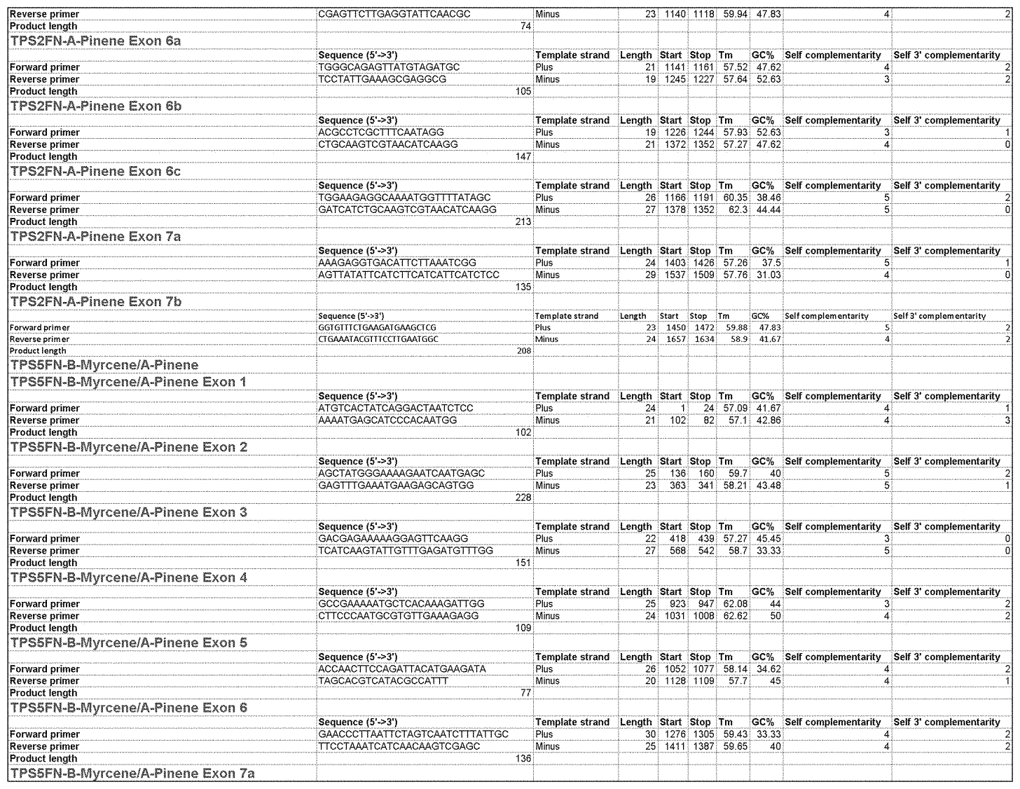

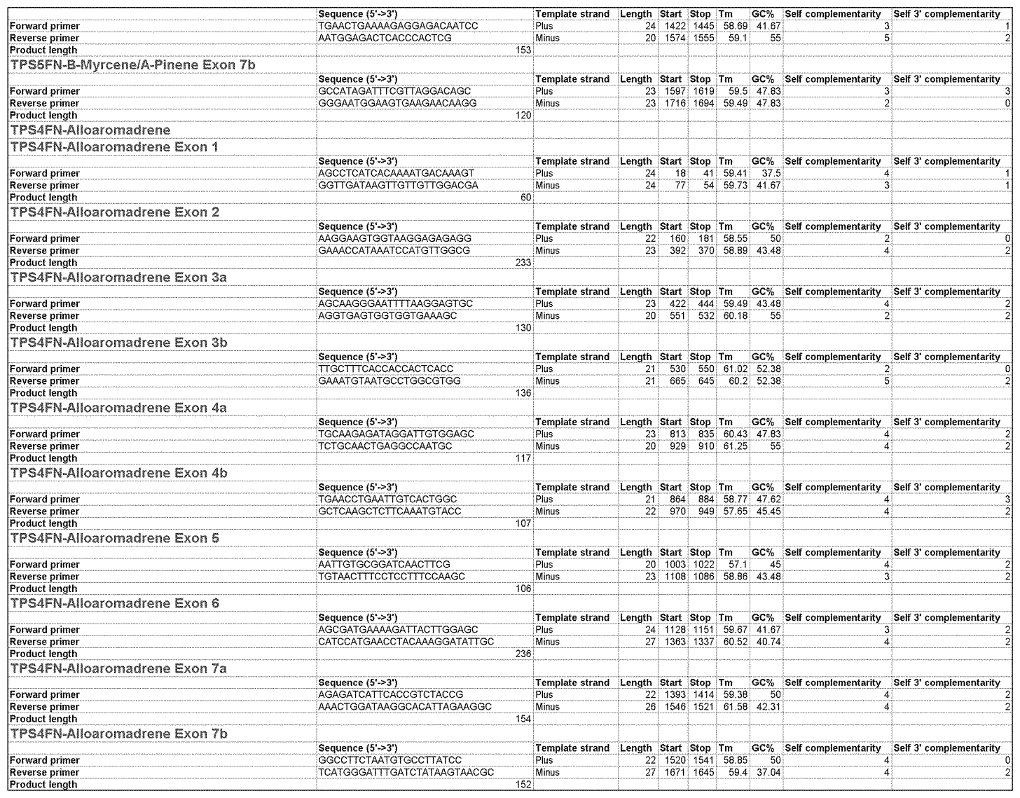

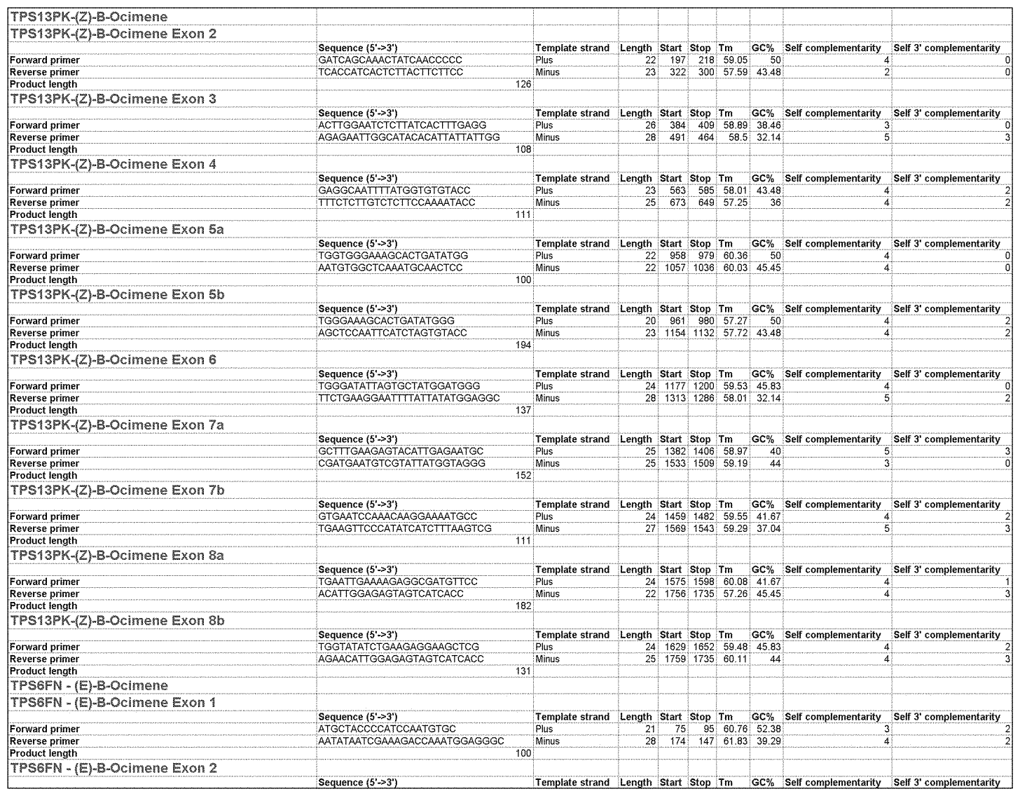

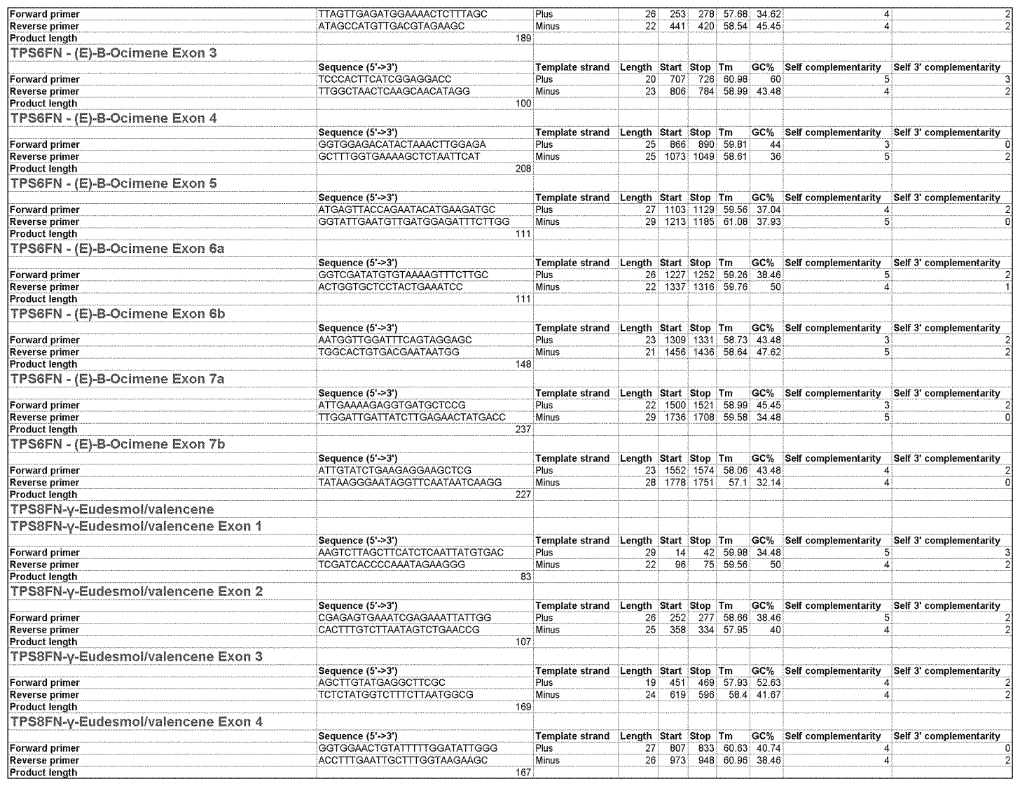

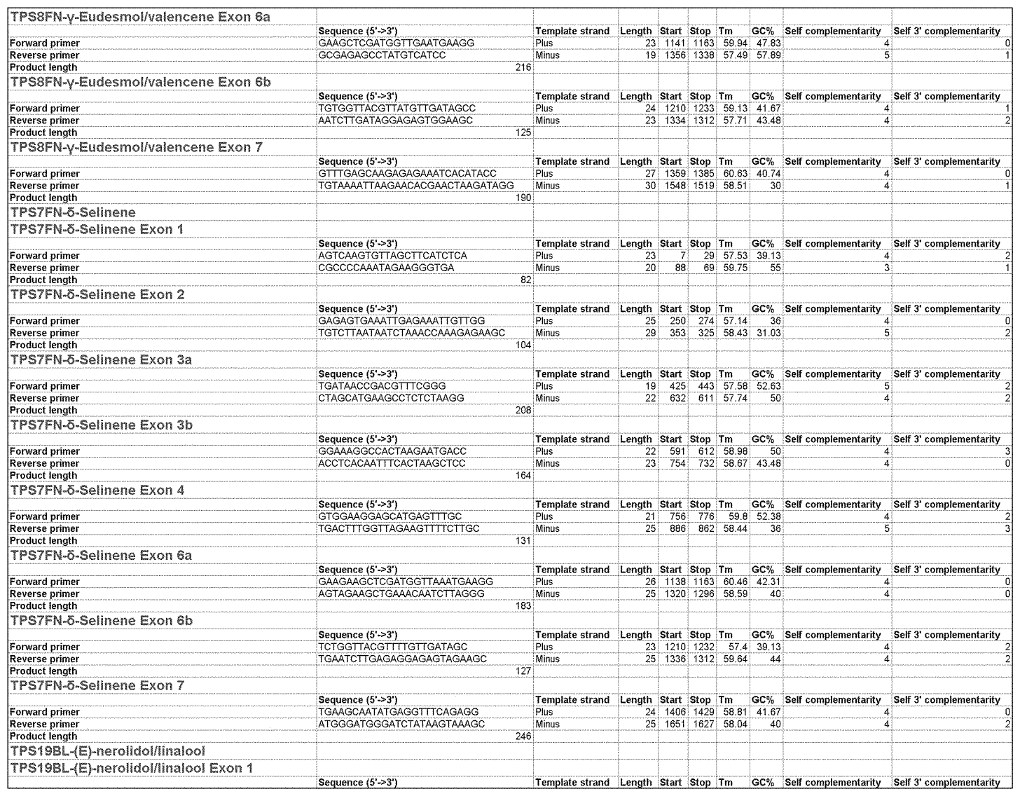

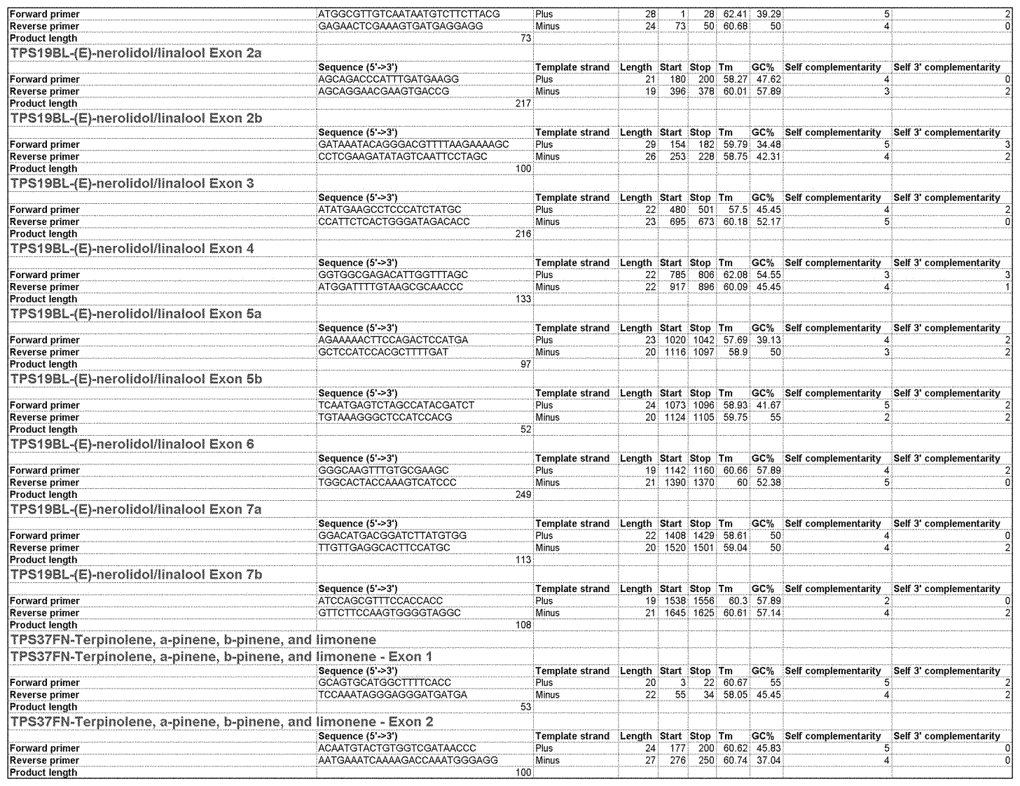

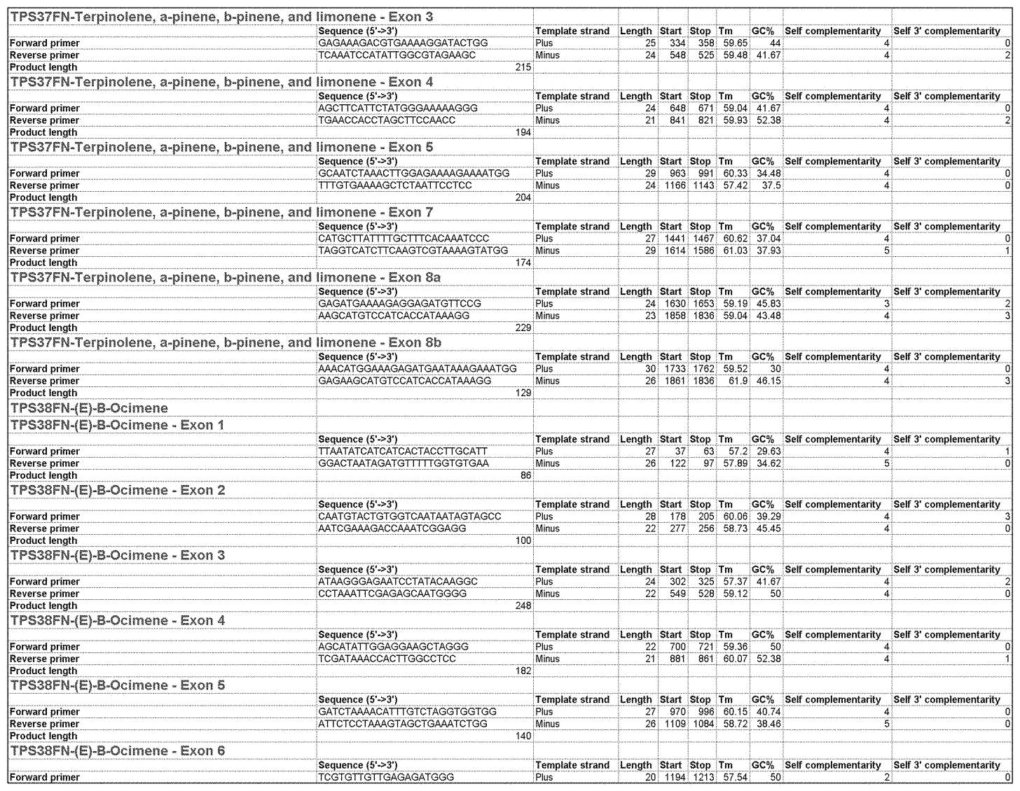

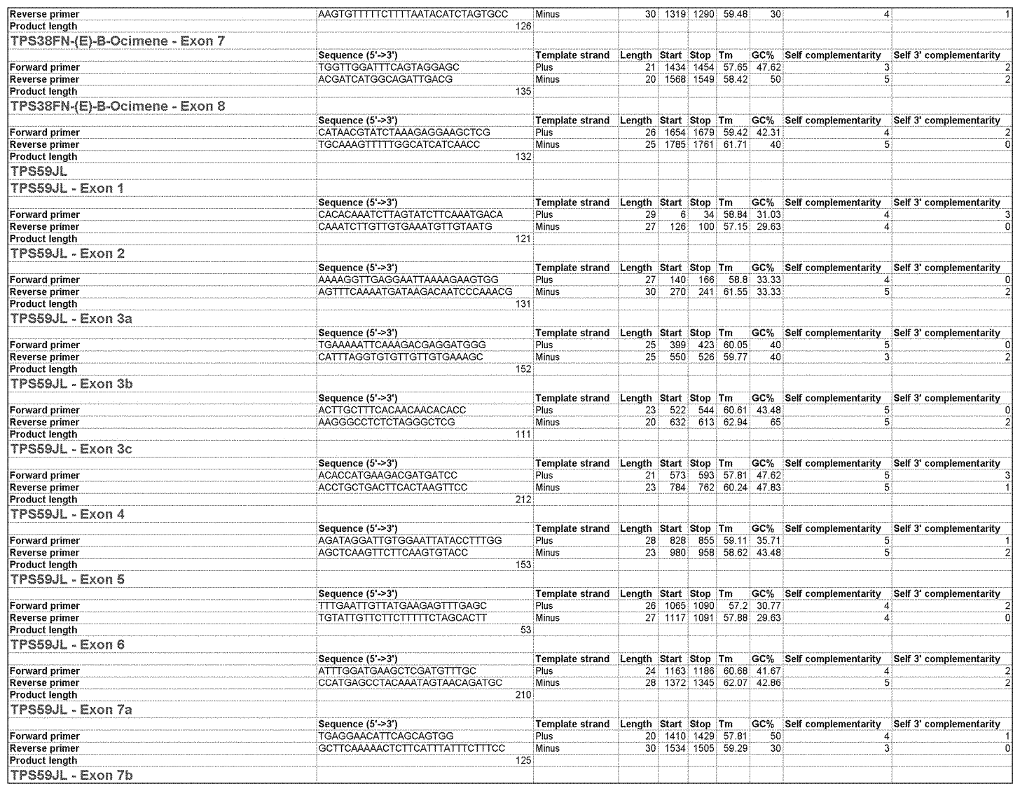

Generally, a primer pair includes a forward primer and a reverse primer. Examples of primer pairs that can be used to detect exons in 74 Cannabis sativa TPS genes are set forth in Table 2 (SEQ ID NOS: 1-1284). Two primer pairs can include two different forward primer species (e.g., A-fwd and B-fwd) and two different reverse primer species (e.g., A-rev, B-rev); can include one forward primer species (e.g., A-fwd) and two different reverse primer species (e.g., A-rev, B-rev); or can include two different forward primer species (e.g., A-fwd and B-fwd) and one reverse primer species (e.g., A-rev), provided the combination of forward and reverse primer species is capable of generating two amplification product species. Further forward and reverse primer combinations are contemplated for additional primer pairs. Examples of forward and reverse primer pairing combinations, with the corresponding amplification product species, is provided in FIG. 1 herein. For example, the size of the product (amplicon) that is amplified by any of the pairs of primers provided herein, including primers prepared by any of the methods provided herein, can be, e.g., about 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290 or 295 base pairs, within +/−about 10% of each of the recited sizes of the amplicons. For example, the sizes of the primers used to generate the amplicons can be between about 10 bases to about 50 bases, generally between about 12, 13, 14 or 15 bases to about 20, 21, 22, 24, 25, 26, 27, 28, 29, 30, 31, 32, 33, 34 or 35 or more bases, for example between 15 to about 30 bases, such as 15, 16, 17, 18, 19, 20, 21, 22, 23, 24, 25, 26, 27, 28, 29 or 30 bases.

In certain embodiments, when a plurality of primer pairs is used, either in a single reaction container or in a separate reaction container for each primer pair, a majority of the polynucleotide primer pairs hybridize to subsequences of the TPS genes and/or paralogs thereof of the plant sample. A majority of the polynucleotide primer pairs can refer to greater than 50% of the primer pairs. For example, a majority of the polynucleotide primer pairs can refer to greater than 60% of the primer pairs, greater than 70% of the primer pairs, greater than 80% of the primer pairs, or greater than 90% of the primer pairs. In embodiments, all (e.g., 100%) of the polynucleotide primer pairs hybridize to subsequences of the TPS genes and/or paralogs thereof of a plant sample. In certain embodiments, the primer pairs are selected from among those set forth in Table 2, or from among those set forth in FIG. 1. Any of the forward primers set forth in Table 2 and FIG. 1 can be paired with any of the reverse primers set forth in Table 2 and FIG. 1, to form primer pairs for use in the methods herein.

In certain embodiments, one or more of the unique subsequences to which the polynucleotide primers hybridize can contain one or more variant nucleotide position, such as a substitution, an insertion or a deletion i.e., the methods of analysis provided herein can detect a genetic modification in a TPS gene.

A unique subsequence of a TPS gene or paralog thereof, to which the primer pairs hybridize, can be referred to as a target sequence. A target sequence generally refers to a unique subsequence, such as an exon, of a TPS gene or paralog thereof, between the two hybridization sites of a corresponding primer pair, and generally does not include the primer hybridization sites themselves. In embodiments, the two primer hybridization sites are conserved sequence regions that flank a diverse sequence, i.e., a unique subsequence of a TPS gene or paralog thereof is diverse and can differ from other subsequences of the TPS gene and of other TPS genes by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 or more bases, such as 110, 120, 130, 140 or 150 or more bases. In embodiments, the variant positions described for a target sequence do not include positions in the primer hybridization sites. In certain embodiments, the TPS genes and/or paralogs thereof have an overall sequence identity of percentages from between about 40% to about 90, 91, 92, 93, 94, 95, 96, 97, 98, 99 or 100% , such as at least 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In embodiments, one or more plant cultivars are of the family Rosidae. In certain embodiments, the plant sample is from a Cannabis cultivar, and the TPS gene profile is of a Cannabis genome. In aspects, tthe Cannabis cultivar is selected from among one or more of Type 1, Type 2, Type 3, Type 4 and Type 5 cultivars. Examples of Cannabis genomes include, but are not limited to, a Cannabis sativa genome, Cannabis indica genome, or Cannabis ruderalis genome. Examples of Cannabis genomes include CS10 (GENBANK assembly accession: GCA_900626175.1; REFSEQ assembly accession: GCF_900626175.1), Arcata Trainwreck, Grape Stomper, Citrix, Black 84, Headcheese, Red Eye OG, Tahoe OG, Master Kush, Chem 91, Domnesia, Sour Tsunami, Sour Tsunami_x_CK, Tibor_1_2016, 80 E-1, 80 E-2, 80 E-3, Harlox, Saint Jack, Herijuana, Mothers Milk_5, Black Beauty, Sour Diesel, JL_1, JL_2, JL_3, JL_4, JL_5, JL_6, JL_father, BBCC_x_JL_father, JL_mother, JL_mother_p, IdaliaFT_1, Fedora_16_6_1, Carmal_1_2016, CS_1_2016, EICam_1_2016, C3/USO-1, Carmagnola_3, and Merino_S_1.

A subsequence (e.g., exon) that is non-identical to any subsequence, or complement thereof, in the TPS gene or paralog thereof of a Cannabis genome generally refers to a sequence containing one or more variant nucleotides when compared to any other subsequence, or complement thereof, in the same TPS gene or in other TPS genes of the Cannabis genome. The primers provided herein generally share a high degree of sequence identity to the regions of the subsequence to which they hybridize. In some embodiments, each polynucleotide in each primer pair contains a sequence that is at least about 95% identical to a subsequence, or complement thereof, of a TPS gene in the genome of the plant cultivar. In certain embodiments, each polynucleotide in each primer pair contains a sequence that is 100% identical to a subsequence, or complement thereof, of a TPS gene in the genome of the plant cultivar.

In some embodiments, a primer provided herein includes a polynucleotide where one or more nucleotide positions contain a nonstandard nucleotide and/or a degenerate nucleotide. A nonstandard nucleotide can be, for example, a non-natural base, a modified base, or a universal base. A universal base is a base capable of indiscriminately base pairing with each of the four standard nucleotide bases: A, C, G and T. Universal bases that may be incorporated into a primer herein include, but are not limited to, inosine, deoxyinosine, 2′-deoxyinosine (dl, dlnosine), nitroindole, 5-nitroindole, and 3-nitropyrrole (e.g., 5′ nitroindole, deoxyinosine, deoxynebularine). A degenerate nucleotide typically refers to a mixture of nucleotides at a given position and may be represented by a letter other than A, T, G or C. For example, a degenerate nucleotide may be represented by R (A or G), Y (C or T), S (G or C), W (A or T), K (G or T), M (A or C), B (C or G or T), D (A or G or T), H (A or C or T), V (A or C or G), or N (any base), for example. Such symbols for degenerate nucleotides are part of the International Union of Pure and Applied Chemistry (IUPAC) standard nomenclature for nucleotide base sequence names and represent degenerate or nonstandard nucleotides that can bind multiple nucleotides. For example, an “M” in a primer or probe would include a mixture of A and C at that position, and thus could bind to either T or G in a complementary DNA strand. An “N” in a primer or probe would include a mixture of A, T, G and C at that position, and thus could bind to any nucleotide at that position in the complementary DNA strand.

Methods for Analyzing Nucleic Acids

Provided herein are methods for analyzing nucleic acids. In embodiments, methods herein include analyzing nucleic acid from a plant sample. In certain embodiments, methods provided herein include analyzing nucleic acid from a Cannabis plant sample. In certain embodiments, the methods provided herein include analyzing subsequences of TPS genes and/or paralogs thereof.

In embodiments, analyzing includes detecting the presence or absence of a TPS gene or a paralog thereof in the genome of a plant cultivar. In certain embodiments, analyzing includes determining the presence or absence of more than one TPS gene or a paralog thereof in the genome of a plant cultivar. In embodiments, analyzing includes determining all the TPS genes and/or paralogs thereof that are present in the genome of a plant cultivar. In certain embodiments (e.g., by analyzing cDNA from the plant sample to detect the presence or absence of TPS genes and/or paralogs thereof), the expression profile of TPS genes and/or paralogs thereof in a plant sample can be analyzed. In embodiments, analyzing includes determining the presence or absence of one or more TPS genes and/or paralogs thereof in genomic DNA from the plant cultivar sample. In embodiments, the plant sample is from a Cannabis plant cultivar. In certain embodiments, the presence or absence of a TPS gene or paralog thereof can be determined based on one or more amplification products generated using one or more primer pairs that specifically amplify unique subsequences of one or more TPS genes or paralogs thereof. In certain embodiments, the presence or absence of a TPS gene or paralog thereof can be determined based on two or more amplification products generated using one or more primer pairs that specifically amplify unique subsequences of one or more TPS genes or paralogs thereof. In embodiments, the presence or absence of a TPS gene or paralog thereof can be determined based on 3, 4, 5, 6, 7, 8, 9, 10, 20, 30, 35, 40, 45, 50, 55, 60, 70, 80, 90, 100, 150, 200, 250, 300, 350, 400, 450, 500, 550, 600 or 650 or more amplification products generated using one or more primer pairs that specifically amplify unique subsequences of one or more TPS genes or paralogs thereof. In certain embodiments, the number of TPS genes and/or paralogs thereof that are detected in the nucleic acid from the plant sample can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 or more genes and/or paralogs. In certain embodiments, the plant cultivar is a Cannabis cultivar.

In certain embodiments, analyzing includes detecting a variant of a TPS gene or a paralog thereof in the genome of a plant cultivar, when compared to a reference unmodified genome of the plant cultivar. In embodiments, one or more TPS genes and/or paralogs thereof in the TPS gene profile is modified by genetic modification methods to obtain desired terpene, cannabinoid and/or flavonoid production profiles, and the analyzing includes screening to identify whether the genetic modification is in fact present, when compared to a reference unmodified genome of the plant cultivar. For example, based on the analysis of a reference unmodified TPS gene profile, and the terpene (and/or flavonoid and/or cannabinoid) abundance profile that is expected or is obtained for the unmodified TPS gene profile, it may be desirable to genetically modify one or TPS genes and/or paralogs thereof to provide an improved terpene (and/or flavonoid and/or cannabinoid) abundance profile, e.g., to impart improved medicinal properties, or improved resistance to an organism or environment, or improved affinity for an organism or environment. The variant can include, e.g., one or more nucleotide substitutions, insertions, or deletions at one or more variant positions, thereby changing the terpene and/or cannabinoid and/or flavonoid profiles. Methods of genetically modifying nucleic acids are known to those of skill in the art and include, but are not limited to, ZFN (Zinc Finger Nuclease), TALEN (Transcription Activator-Like Effector Nucleases), CRISPR-cas (cas9, cas12, cas13), Cre-Lox, MiRNA, SiRNA, ShRNA or a combination thereof. In certain embodiments, analyzing includes determining a terpene abundance profile, a flavonoid abundance profile, or any combination thereof. Techniques for measuring terpenes include, but are not limited to, gas chromatography with a flame ionization detector (GC-FID), gas chromatography—mass spectrometry (GC-MS) and headspace solid-phase microextraction (HS-SPME) in conjunction with GC-MS. Techniques for measuring flavonoids include, but are not limited to, gas chromatography (GC), gas chromatography—mass spectrometry (GC-MS), HPLC, HPLC-UV and NIR (near infrared reflectance). Techniques for measuring cannabinoids include, but are not limited to, HPLC, ultra-HPLC, HPLC-UV, HPLC-MS, UHPLC-MS, time-of-flight mass spectrometry (TOF-MS), LC-TOF-MS and NIR (near infrared reflectance).

In embodiments, detecting one or more genetic variations in a TPS gene or paralog thereof includes contacting the nucleic acid of the plant sample with one or more primer pairs as provided herein, under conditions wherein the one or more primer pairs hybridize to the one or more unique subsequences of a TPS gene or paralog thereof, wherein the one or more unique subsequences contain one or more variant nucleotide positions relative to the corresponding wild-type or unmodified subsequence in the plant cultivar. Following hybridization, the amplification conditions can be the same amplification conditions as those used to amplify the corresponding wild-type or unmodified subsequence, or they can be a different set of amplification conditions. In embodiments, a set of primers can be designed to hybridize with greater specificity for the expected genetically modified variant sequence.

Any suitable method for genotype assessment may be used for detecting a genetic variation in a TPS gene and/or paralog thereof, such as, for example, nucleic acid sequencing (examples of which are described herein) and/or a high-resolution melting (HRM) assay provided herein. Generally, a sequencing process and/or an HRM assay are performed in conjunction with a nucleic acid amplification method described herein (e.g., using the amplification primers provided herein). In certain embodiments, one or more genetic variations can be determined based on the presence and/or absence of amplification products generated using certain amplification primers provided herein.

Samples

Provided herein are methods and compositions for processing, preparing, and/or analyzing nucleic acid. Nucleic acid or a nucleic acid mixture used in the methods and compositions described herein can be isolated from a sample (e.g., a test sample) obtained from a plant cultivar. A plant cultivar can be any plant whose genome includes TPS synthase genes and/or that produces terpenes, including for example, angiosperms, any species of woody, ornamental or decorative, crop or cereal, fruit or vegetable, fruit plant or vegetable plant, flower or tree, macroalga or microalga, phytoplankton and photosynthetic algae (e.g., green algae Chlamydomonas reinhardtii). A plant also refers to a unicellular plant (e.g. microalga) and a plurality of plant cells that are largely differentiated into a colony (e.g. volvox) or a structure that is present at any stage of a plant's development. Such structures include, but are not limited to, a fruit, a flower, a seed, a shoot, a stem, a leaf, a root, plant tissue sand the like. As used herein, the term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue can be in planta, in organ culture, tissue culture, or cell culture. Any of the foregoing plant cultivars, portions thereof or extracts thereof are contemplated for use in the methods provided herein.

A nucleic acid sample can be isolated, obtained or prepared from any type of suitable biological (e.g., plant) specimen or sample (e.g., a test sample). A nucleic acid sample can be isolated or obtained from a single plant cell, a plurality of plant cells (e.g., cultured plant cells), plant cell culture media, conditioned plant cell culture media, or plant tissue (e.g., leaves, roots, stems).

A sample can be heterogeneous. For example, a sample can include more than one cell type and/or one or more nucleic acid species. In embodiments, a sample can include plant nucleic acid from more than one plant cultivar. In embodiments, the more than one plant cultivar providing the nucleic acid belong to the same species, e.g., both can be Cannabis cultivars. In embodiments, a sample can include plant cells and/or nucleic acid from a single plant or can include plant cells and/or nucleic acid from multiple plants.

Nucleic Acid

Provided herein are methods and compositions for processing, preparing, and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (IncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. The plant nucleic acid analyzed according to the methods provided herein can be from, a plant, a plasmid containing plant nucleic acid, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term “nucleic acid” includes nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions can be achieved by generating sequences in which the third position of one or more selected (or all) codons is substituted with mixed-base and/or deoxyinosine residues. The term nucleic acid can be used interchangeably herein with locus, gene, cDNA, and mRNA encoded by a gene. The term also can include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” also can refer to a section of DNA involved in producing a polypeptide chain, such as an exon or portion thereof; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size can be expressed as a number of bases.

Target nucleic acids, such as a TPS gene or a paralog thereof or a portion thereof containing a unique subsequence, can be any nucleic acids of interest. Nucleic acids can be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid can be single or double stranded. Single stranded DNA (ssDNA), for example, can be generated by denaturing double stranded DNA by heating or by treatment with alkali, for example. Accordingly, in some embodiments, ssDNA is derived from double-stranded DNA (dsDNA).

Nucleic acid (e.g., nucleic acid targets, polynucleotides, primers, polynucleotide primers, polynucleotide primer pairs, sequences, and subsequences) as described herein can be complementary to another nucleic acid, hybridize to another nucleic acid, and/or be capable of hybridizing to another nucleic acid. The terms “complementary” or “complementarity” or “hybridization” generally refer to a nucleotide sequence that base-pairs by non-covalent bonds to a region of a nucleic acid (e.g., a primer that hybridizes to a unique subsequence of a TPS gene or a paralog thereof). In the canonical Watson-Crick base pairing, adenine (A) forms a base pair with thymine (T), and guanine (G) pairs with cytosine (C) in DNA. In RNA, thymine (T) is replaced by uracil (U). Thus, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “hybridize” or “capable of hybridizing” refers to a nucleotide sequence that is at least partially complementary. These terms can also encompass duplexes that are fully complementary such that every nucleotide in one strand is complementary or hybridizes to every nucleotide in the other strand in corresponding positions.

In certain instances, a nucleotide sequence can be partially complementary to a target, wherein not all nucleotides of, e.g., a primer, are complementary to every nucleotide in the target nucleic acid (unique subsequence, e.g., exon of a TPS synthase gene or paralog thereof) in all the corresponding positions. For example, the primer can be perfectly (i.e., 100%) complementary to a unique subsequence of a TPS synthase gene or paralog thereof, or a primer can share some degree of complementarity to a unique subsequence of a TPS synthase gene or paralog thereof, e.g., 70%, 75%, 85%, 90%, 95%, 99%.

The percent identity of two nucleotide sequences can be determined by aligning the sequences for optimal comparison purposes (e.g., gaps can be introduced in the sequence of a first sequence for optimal alignment) The nucleotides at corresponding positions are then compared, and the percent identity between the two sequences can be determined as a function of the number of identical positions shared by the sequences (i.e., % identity=# of identical positions/total # of positions×100). When a position in one sequence is occupied by the same nucleotide as the corresponding position in the other sequence, then the molecules are identical at that position.

In certain embodiments, nucleic acids in a mixture of nucleic acids are analyzed. A mixture of nucleic acids can include two or more nucleic acid species having the same or different nucleotide sequences, different lengths, different origins (e.g., genomic origins, cDNA, cell or tissue origins, sample origins, subject origins, and the like), different amplification products (e.g., amplification products generated from different sets of primer pairs), or combinations thereof. In certain embodiments, a mixture of nucleic acids includes or can generate a plurality of amplification product species generated from different sets of primer pairs (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 150 or more, 200 or more, 250 or more, 300 or more, 350 or more, 400 or more, 450 or more, 500 or more, 550 or more, 600 or more, or 650 or more amplification product species). In embodiments, a mixture of nucleic acids includes single-stranded nucleic acid and double-stranded nucleic acid. In certain embodiments, a mixture of nucleic acids includes DNA and RNA. In certain embodiments, a mixture of nucleic acids includes ribosomal RNA (rRNA) and messenger RNA (mRNA).

Nucleic acids used in the methods provided herein can contain nucleic acid from one plant sample or from two or more plant samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more plant samples).

Nucleic acid can be derived from one or more plant sources by methods known in the art. Any suitable method can be used for isolating, extracting and/or purifying DNA from a plant sample, non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QlAprep®, QlAquick®, and QlAamp®, nucleic acid isolation/purification kits by Qiagen, Inc. (Germantown, Md.); DNAzol®, ChargeSwitch®, Purelink®, GeneCatcher® nucleic acid isolation/purification kits by Life Technologies, Inc. (Carlsbad, Calif.); NucleoMag®, NucleoSpin®, and NucleoBond® nucleic acid isolation/purification kits by Clontech Laboratories, Inc. (Mountain View, Calif.), DNA/RNA extraction kits from Zymo Research (e.g., ZYMOBIOMICS DNA Mini Kit, ZYMOBIOMICS DNA/RNA Miniprep Kit, ZYMOCLEAN gel DNA recovery); the like or combinations thereof.

Nucleic acid can be provided for performing methods described herein with or without processing of the sample(s) containing the nucleic acid. In embodiments, nucleic acid is provided for performing methods provided herein after processing of the sample(s) containing the nucleic acid. For example, a nucleic acid can be extracted, isolated, purified, partially purified and/or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a test subject (e.g., a plant). An isolated nucleic acid can be provided with fewer non-nucleic acid components (e.g., protein, lipid) than the number of components present in a source sample. A composition containing isolated nucleic acid can be about 50% to greater than 99% free of non-nucleic acid components. A composition containing isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the number of non-nucleic acid components present prior to subjecting the nucleic acid to a purification and/or analysis procedure. A composition containing purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition containing purified nucleic acid may be about 90%, 91%, 92%, 93%, 94%, 95%, 96%, 97%, 98%, 99% or greater than 99% free of nucleic acid species other than the plant nucleic acid of interest.

In certain embodiments, nucleic acid for performing methods provided herein is used without prior processing of the sample(s) containing the nucleic acid. For example, nucleic acid can be analyzed directly from a plant sample without prior extraction, purification, partial purification, and/or amplification.

Nucleic acid also can be exposed to a process that modifies certain nucleotides in the nucleic acid before being analyzed or prepared according to the methods provided herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid can be provided in any form that is suitable for conducting an analysis (e.g., genotype analysis, sequence analysis).

Primers

Primers useful for detection, amplification, quantification, sequencing and/or analysis of nucleic acid are provided. The term “primer” as used herein refers to a nucleic acid that includes a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence), or feature thereof, for example. A primer typically is a synthetic sequence. The term “specific” or “specificity,” as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the terms “anneal” and “hybridize” refer to the formation of a stable complex between two molecules. The terms “primer,” “polynucleotide,” “oligo,” or “oligonucleotide” are used interchangeably herein, when referring to primers.

A primer nucleic acid can be designed and synthesized using methods known to those of skill in the art as well as those provided herein. The primers used in the methods provided herein can be of any length suitable for hybridizing to a nucleotide sequence of interest (e.g., where the nucleic acid is in liquid phase or is bound to a solid support) and performing methods of analyses described herein. Primers can be designed based on any target nucleotide sequence, such as a unique subsequence of a TPA gene or a paralog thereof. A primer, in embodiments, can be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer can include naturally occurring and/or non-naturally occurring nucleotides (e.g., labeled nucleotides), or a mixture thereof. Primers suitable for use in the methods provided herein can be synthesized and labeled using known techniques. For example, primers can be chemically synthesized according to the solid phase phosphoramidite triester method first described by Beaucage and Caruthers, Tetrahedron Lett., 22:1859-1862, 1981, using an automated synthesizer, as described in Needham-VanDevanter et al., Nucleic Acids Res. 12:6159-6168, 1984. Purification of primers can be achieved by native acrylamide gel electrophoresis or by anion-exchange high-performance liquid chromatography (HPLC), for example, as described in Pearson and Regnier, J. Chrom., 255:137-149, 1983.

All or a portion of a primer sequence can be complementary or substantially complementary to a target nucleic acid. As referred to herein, “substantially complementary” with respect to sequences refers to nucleotide sequences that will hybridize with each other. The stringency of the hybridization conditions can be altered to tolerate varying amounts of sequence mismatch. Included are target and primer sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Primers that are substantially complimentary to a target nucleic acid sequence are also substantially identical to the compliment of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Primer sequences and length can affect hybridization to target nucleic acid sequences. Depending on the degree of mismatch between the primer and target nucleic acid, low, medium or high stringency conditions may be used to effect primer/target annealing. s used herein, the term “stringent conditions” refers to conditions for hybridization and washing. Methods for hybridization reaction temperature condition optimization are known, and can be found, e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6× sodium chloride/sodium citrate (SSC) at about 45° C., followed by one or more washes in 0.2× SSC, 0.1% SDS at 50° C. Another example of stringent hybridization conditions includes hybridization in 6× sodium chloride/sodium citrate (SSC) at about 45° C., followed by one or more washes in 0.2× SSC, 0.1% SDS at 55° C. A further example of stringent hybridization conditions includes hybridization in 6× sodium chloride/sodium citrate (SSC) at about 45° C., followed by one or more washes in 0.2× SSC, 0.1% SDS at 60° C. Often, stringent hybridization conditions are hybridization in 6× sodium chloride/sodium citrate (SSC) at about 45° C., followed by one or more washes in 0.2× SSC, 0.1% SDS at 65° C. More often, stringency conditions can include 0.5 M sodium phosphate, 7% SDS at 65° C., followed by one or more washes at 0.2× SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids. Features of primers described herein also can apply to probes such as, for example, the qPCR probes provided herein.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

In certain embodiments, primers can include a nucleotide subsequence that is complementary to a solid phase nucleic acid primer hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). A primer can contain a nucleotide subsequence not complementary to or not substantially complementary to a solid phase nucleic acid primer hybridization sequence (e.g., a sequence at the 3′ or 5′ end of the nucleotide subsequence in the primer complementary to or substantially complementary to the solid phase primer hybridization sequence, which sequence can hybridize to a unique subsequence in a TPS gene or paralog thereof).

A primer, in certain embodiments, can contain a modification such as one or more nonstandard nucleotides, non-natural nucleotides, universal bases, degenerate nucleotides, inosines, abasic sites, locked nucleic acids, minor groove binders, duplex stabilizers (e.g., acridine, spermidine), Tm modifiers or any modifier that changes the binding properties of the primers or probes. A primer, in certain embodiments, can contain a detectable molecule or entity (e.g., a fluorophore, radioisotope, colorimetric agent, particle, enzyme, and the like).

A primer also can refer to a polynucleotide sequence that, when hybridized to a subsequence of a target nucleic acid or another primer, facilitates the detection of a primer, a target nucleic acid or both, as with molecular beacons, for example. The term “molecular beacon,” as used herein, refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

Amplification

Nucleic acids can be amplified under amplification conditions. The terms “amplify,” “amplification,” “amplification reaction,” “amplifying,” “amplified,” or “amplification conditions” as used herein refer to subjecting a target nucleic acid in a plant sample (e.g., TPS genes or paralogs thereof in a plant cultivar genome, or plant cDNA) to a process that linearly or exponentially generates amplicon nucleic acids having the same or substantially the same nucleotide sequence as the target nucleic acid or a portion thereof. In certain embodiments, the term “amplified” or “amplification” or “amplification conditions” refers to a method that includes a polymerase chain reaction (PCR). Nucleic acid can be amplified using a suitable amplification process. Nucleic acid amplification typically involves enzymatic synthesis of nucleic acid amplicons (copies), which contain a sequence complementary to a nucleotide sequence being amplified.

In certain embodiments, a limited amplification reaction, also known as pre-amplification, can be performed (e.g., of gDNA). Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification can limit inaccuracies associated with depleted reactants in standard PCR reactions, for example, and also can reduce amplification biases due to nucleotide sequence or species abundance of the target. In embodiments, a one-time primer extension can be performed as a prelude to linear or exponential amplification.

Any suitable amplification technique can be utilized. Amplification methods include, but are not limited to, polymerase chain reaction (PCR); ligation amplification (or ligase chain reaction (LCR)); amplification methods based on the use of Q-beta replicase or template-dependent polymerase (e.g., U.S. Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence-based amplification (3SR or NASBA), and transcription-associated amplification (TAA). Non-limiting examples of PCR amplification methods include standard PCR, AFLP-PCR, allele-specific PCR, Alu-PCR, asymmetric PCR, colony PCR, hot start PCR, inverse PCR (IPCR), in situ PCR (ISH), intersequence-specific PCR (ISSR-PCR), long PCR, multiplex PCR, nested PCR, quantitative PCR (qPCR), touchdown PCR, reverse transcriptase PCR (RT-PCR), reverse transcriptase quantitative PCR (RT-qPCR), TAQMAN qPCR, real time PCR, single cell PCR, solid phase PCR, combinations thereof, and the like. Reagents and hardware for conducting PCR are commercially available.

It is understood by those of skill in the art that modifications to these PCR protocols can be made to achieve the same or similar results. For example, the temperatures for the various steps in the can be modified by between about 1-5° C., or touchdown PCR can be performed, i.e., the annealing temperature is adjusted based on the cycle number.

A generalized description of an amplification process is as follows. Primers and target nucleic acid are contacted, and complementary sequences hybridize to one another, for example. Primers can hybridize to a target nucleic acid, at or near (e.g., adjacent to, abutting, and the like) a sequence of interest. A reaction mixture, containing components necessary for enzymatic functionality, is added to the primer-target nucleic acid hybrid, and amplification can occur under suitable conditions. Components of an amplification reaction can include, but are not limited to, e.g., primers (e.g., individual primers, primer pairs, a plurality of primer pairs, and the like) a polynucleotide template (e.g., target nucleic acid), polymerase, nucleotides, dNTPs and the like. In embodiments, non-naturally occurring nucleotides or nucleotide analogs, such as analogs containing a detectable label (e.g., fluorescent or colorimetric label), can be used for example.

Any suitable polymerase can be selected, which can include polymerases for thermocycle amplification (e.g., Taq DNA Polymerase; Q-Bio™ Taq DNA Polymerase (recombinant truncated form of Taq DNA Polymerase lacking 5′-3′exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

PCR conditions can be dependent upon primer sequences, target abundance, and the desired amount of amplification, and therefore, any suitable PCR protocol may be selected. PCR is typically carried out as an automated process with a thermostable enzyme. In this process, the temperature of the reaction mixture is cycled through a denaturing step, a primer-annealing step, and an extension reaction step automatically. Some PCR protocols also include an activation step and a final extension step. Machines specifically adapted for this purpose are commercially available. A non-limiting example of a PCR protocol that may be suitable for embodiments described herein is as follows: treating the sample at 95° C. for 2 minutes; repeating 40 cycles of 95° C. for 15 seconds and 60° C. for 30 seconds. Additional examples of suitable PCR protocols are provided in the working examples herein. A completed PCR reaction can optionally be kept at 4° C. until further action is desired. Multiple cycles frequently are performed using a commercially available thermal cycler. Suitable isothermal amplification processes also can be applied, in certain embodiments.

In certain embodiments, an amplification product can include naturally occurring nucleotides, non-naturally occurring nucleotides, nucleotide analogs and the like and combinations of the foregoing. An amplification product often has a nucleotide sequence that is identical to or substantially identical to a sample nucleic acid nucleotide sequence or complement thereof. A “substantially identical” nucleotide sequence in an amplification product will generally have a high degree of sequence identity to the nucleotide sequence species being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

In embodiments, where a target nucleic acid is RNA, prior to the amplification step, a DNA copy (cDNA) of the RNA transcript of interest may be synthesized. A cDNA can be synthesized by reverse transcription, which can be carried out as a separate step, or in a homogeneous reverse transcription-polymerase chain reaction (RT-PCR), a modification of the polymerase chain reaction for amplifying RNA.

Amplification also can be accomplished using digital PCR, in certain embodiments. Digital PCR takes advantage of nucleic acid (DNA, cDNA or RNA) amplification on a single molecule level and offers a highly sensitive method for quantifying low copy number nucleic acid. Systems for digital amplification and analysis of nucleic acids are available (e.g., Fluidigm® Corporation).

Amplification reactions can be performed as individual amplification reactions, where one primer pair is used for each reaction and the presence or absence of one amplification product is detected. In certain embodiments, multiple individual amplification reactions may be performed (i.e., carried out in separate containers) using a different set of primers for each reaction, and the presence or absence of an amplification product is detected for each individual reaction. In embodiments, amplification reactions are performed as multiplex amplification reactions (i.e., a plurality of amplification reactions performed in a single container), where a plurality of primer pairs is used for the multiplex reaction, and the presence or absence of more than one amplification product is detected. Both individual amplification reactions and multiplex amplification reactions are contemplated for the primers provided herein.

In certain embodiments, a method provided herein includes generating nucleic acid amplification products from a plant sample. Such methods include contacting nucleic acid of a plant sample with a pair of polynucleotide primers under conditions wherein the pair of polynucleotide primers hybridize to and amplify a unique subsequence, when present, in a TPS gene or a paralog thereof in the genome (or cDNA, e.g., for obtaining an expression profile) of a plant cultivar.

Quantitative PCR

In certain embodiments, an amplification method includes a quantifiable amplification method. For example, levels of expression of a TPS synthase gene or a paralog thereof can be measured using a quantitative PCR (qPCR) approach (e.g., on cDNA generated from RNA from a plant sample), or a reverse transcriptase quantitative PCR (RT-qPCR) approach (e.g., on RNA from a plant sample). Quantitative PCR (qPCR), which also can be referred to a real-time PCR, monitors the amplification of a targeted nucleic acid molecule during a PCR reaction (i.e., in real time). This method can be used quantitatively (quantitative real-time PCR) and semi-quantitatively (i.e., above/below a certain amount of nucleic acid molecules; semi-quantitative real-time PCR). The primers can be gene-specific probes that quantitate each amplicon (i.e., individual TPS genes), or they can be class-specific probes, e.g., to quantitate all monoterpene synthases, all diterpene synthases, all sesquiterpene synthases or combinations thereof in the TPS gene profile of the plant cultivar.

Methods for qPCR include use of non-specific fluorescent dyes that intercalate with double-stranded DNA, and sequence-specific DNA probes labelled with a fluorescent reporter, which generally allows detection after hybridization of the probe with its complementary sequence. Quantitative PCR methods typically are performed in a thermal cycler with the capacity to illuminate each sample with a beam of light of at least one specified wavelength and detect the fluorescence emitted by an excited fluorophore.

For non-specific detection, a DNA-binding dye can bind to all double-stranded (ds) DNA during

PCR. An increase in DNA product during PCR therefore leads to an increase in fluorescence intensity measured at each cycle. For qPCR using dsDNA dyes, the reaction typically is prepared like a basic PCR reaction, with the addition of fluorescent dsDNA dye. Then the reaction is run in a real-time PCR instrument, and after each cycle, the intensity of fluorescence is measured with a detector (the dye only fluoresces when bound to the dsDNA (i.e., the PCR product)). In certain applications, multiple target sequences can be monitored in a tube by using different types of dyes.

For specific detection, fluorescent reporter probes detect only the DNA containing the sequence complementary to the probe. Accordingly, use of the reporter probe can, in embodiments, increase specificity and facilitate performing the technique even in the presence of other dsDNA. Using different types of labels, fluorescent probes can be used in multiplex assays for monitoring several target sequences in the same tube. This method typically uses a DNA-based probe with a fluorescent reporter at one end and a quencher of fluorescence at the opposite end of the probe. The close proximity of the reporter to the quencher prevents detection of its fluorescence. During

PCR, the probe is broken down by the 5′ to 3′ exonuclease activity of the polymerase, which breaks the reporter-quencher proximity and thus permits unquenched emission of fluorescence, which can be detected after excitation with a laser. An increase in the product targeted by the reporter probe at each PCR cycle therefore causes a proportional increase in fluorescence due to the breakdown of the probe and release of the reporter.

In certain embodiments, a method provided herein includes contacting nucleic acid of a plant sample with one or more primer pairs and one or more quantitative PCR probes. For example, certain primers provided herein (e.g., primers provided in Table 2) can be used in combination with certain qPCR probes.

Loop Mediated Isothermal Amplification (LAMP)

In certain embodiments, an amplification method includes loop mediated isothermal amplification (LAMP). Loop-mediated isothermal amplification (LAMP) is a single-tube technique useful for nucleic acid amplification. Reverse transcription loop-mediated isothermal amplification (RT-LAMP) combines LAMP with a reverse transcription step for the detection of RNA. LAMP is typically performed under isothermal conditions. In contrast to a polymerase chain reaction (PCR) technology, which is typically performed using a series of alternating temperature cycles, isothermal amplification is performed at a constant temperature, and does not require a thermal cycler.

In LAMP, a target sequence is amplified at a constant temperature (e.g., between about 60° C. to about 65° C.) using a plurality of primer pairs (e.g., two primer pairs, three primer pairs) and a polymerase (e.g., a polymerase with high strand displacement activity). In certain applications, four different primers can be used to amplify six distinct regions on a target sequence, for example, which can increase specificity. An additional pair of loop primers can further accelerate the reaction.

The amplification product can be detected via photometry (i.e., measuring the turbidity caused by magnesium pyrophosphate precipitate in solution as a byproduct of amplification). This generally allows for visualization by the naked eye or by photometric detection approaches (e.g., for small volumes). In certain applications, the reaction can be followed in real-time either by measuring turbidity or by fluorescence using intercalating dyes (e.g., SYTO 9, SYBR green). Certain dyes can be used to create a visible color change that can be seen with the naked eye without the need for specialized equipment. Dye molecules intercalate or directly label the DNA, and in turn can be correlated with the number of copies initially present. Accordingly, certain variations of LAMP can be quantitative. Detection of LAMP amplification products also can be achieved using manganese loaded calcein, which starts fluorescing upon complexation of manganese by pyrophosphate during in vitro DNA synthesis. Another method for visual detection of LAMP amplification products by the naked eye is based on the ability of the products to hybridize with complementary gold-bound single-stranded DNA, which prevents a red to purple-blue color change that would otherwise occur during salt-induced aggregation of the gold particles.

A number of LAMP visualization technologies are known to those of skill in the art (see, e.g., Fischbach et al., Biotechniques, 58(4):189-194 (2015), the contents of which are incorporated in their entirety by reference herein). Examples of such visualization reagents, summarized in the Table below from Fischbach et al., include magnesium pyrophosphate, hydroxynaphthol blue (HNB), calcein, SYBR Green I, EvaGreen and the nucleic acid-specific dye, berberine, which emits a fluorescent signal under UV light after a positive LAMP reaction.

| | | Hydroxynaphthol | | | | |
|---|---|---|---|---|---|---|
| | Turbidity | blue | Calcein | SYBR Green I | EvaGreen | Berberine |
| Substance | Mg-pyrophosphate | Hydroxynaphthol | Calcein AM + MnCl₂ | SYBR Green I | EvaGreen | Berberine-SO₄ |
| | (Mg-PPi) | blue (HNB) | | | | |
| Origin | Amplification | Synthetic | Synthetic | Synthetic | Synthetic | Natural |
| | product | | | | | |
| Toxicity | None | May cause eye | May be harmful to | Mutation enhancer | Possible carcinogen | May be toxic in high |
| | | irritation | skin and eyes | | | concentrations |
| Detection | Insoluble complex; | Decrease of free | Decrease of free | dsDNA intercalation | dsDNA intercalation | Small groove |
| Mechanism | precipitation | Mg²⁺ | Mn²⁺ | | | intercalation |
| Readout | Turbidity | Absorption | Fluorescence | Fluorescence | Fluorescence | Fluorescence |
| | Absorbance: | Absorbance: | Excitation: 495 nm | Excitation: 494 nm | Excitation: 500 nm | Excitation: 450 nm |
| | 400 nm | 650 nm | Emission: 515 nm | Emission: 521 nm | Emission: 530 nm | Emission: 530 nm |
| Effect on | None | None | Manganese may | Not inhibiting when | Not inhibiting when | Not inhibiting |
| amplification | | | inhibit reaction | used 0.5-1x | used 0.5-1x | (≤180 μM) |
| One-pot | + + | − | + + + | + + + + | + + + + | + + + + |
| real-time | | | | | | |
| assay | | | | | | |
| Equipment for | Turbidometer | − | Fluorometer with | Fluorometer with | Fluorometer with | Fluorometer with |
| real-time | | | FAM filter | FAM filter | FAM filter | FAM filter |
| detection | | | | | | |
| One-pot end | + + + | + + + + | + + + + | − | − | + + + |
| point assay | | | | | | (UV light with low |
| | | | | | | background signal) |
| Equipment for | None (cordless | None | UV lamp | Not applicable | Not applicable | UV lamp |
| end point | centrifuge) | | (optional) | | | |
| detection | | | | | | |
| Evaluation of | + Turbid | + Sky blue | + Green | + Fluorescence | + Fluorescence | + Fluorescence |
| results | − Clear | − Violet | − Orange | signal | signal | signal |
| | | | | − No signal | − No signal | − No signal |
| Percentage of | 0 | <0.1 | <0.1 | 26.01 | 1.18 | 0.82 |
| overall costs * | | | | | | |
| Relative | + + + | + + + | + + + | + + + | + + + | + + + |
| sensitivity | | | | | | |
| * * | | | | | | |
| Field | + + + | + + + + | + + + + | + + | + + | + + + + |
| applicability | | | | | | |
| * * * | | | | | | |

Summary of features relevant for in-the-field loop-mediated isothermal amplification (LAMP) assays tested for detection of potato spindle tuber viroid (PSTVd).
Number of “+” describes the applicability/relevance of the feature.
* Overall costs represent the basic chemicals of common suppliers for one LAMP reaction, dependingon reaction volume.
* * In our assays. Analytical sensitivity may depend on LAMP setup and has to be optimized separately.
* * * Depending on visualization.

In embodiments, a method herein includes contacting nucleic acid of a plant sample with a set of loop mediated isothermal amplification (LAMP) primers. For example, Cannabis plant cultivars, or offspring thereof, containing particular TPS genes can be identified via a LAMP assay. In aspects, the LAMP assay can be a colorimetric assay. Examples of LAMP primer sets that can be used to identify Cannabis plant cultivars, or offspring thereof, containing a terpinolene producing TPS gene (csTPS37FN) are shown in Table 5 below:

TABLE 5
5 LAMP primer sets for Terpinolene-producing gene, csTPS37FN

| | | | dimer (minimum) dG = −2.36 | | | | | |
|---|---|---|---|---|---|---|---|---|
| Set 1 | | 1381-2005 | | | | | GC | |
| label | 5′pos | 3′pos | len | Tm | 5′dG | 3′dG | rate | Sequence (SEQ ID NO) |
| F3 | 134 | 151 | 18 | 57.55 | −4.41 | −4.16 | 0.5 | TCAGTTGGGGGACCAATT (1285) |
| B3 | 332 | 350 | 19 | 56.29 | −5.68 | −4.27 | 0.53 | CATCCGACGATGTTCCTAG (1286) |
| FIP | | | 47 | | | | | GGATCATCATAACCTTCTTCCAAGA-TCTTTTGCATGCTTATTTTGCT |
| | | | | | | | | (1287) |
| BIP | | | 47 | | | | | TGAAGGATCCCTGGAAATATCAAAT-TCTTCAAGTCGTAAAAGTATGG |
| | | | | | | | | (1288) |
| F2 | 154 | 175 | 22 | 57.4 | −3.52 | −4.91 | 0.32 | TCTTTTGCATGCTTATTTTGCT (1289) |
| F1c | 210 | 234 | 25 | 60.23 | −4.76 | −4.86 | 0.4 | GGATCATCATAACCTTCTTCCAAGA (1290) |
| B2 | 306 | 327 | 22 | 55.41 | −4.27 | −4.23 | 0.36 | TCTTCAAGTCGTAAAAGTATGG (1291) |
| B1c | 250 | 274 | 25 | 60.09 | −4.86 | −3.57 | 0.36 | TGAAGGATCCCTGGAAATATCAAAT (1292) |
| LF | 178 | 202 | 25 | 60.48 | −5.7 | −4.33 | 0.4 | GGAAGCTTTTTCTAAGGGATTIGTG (1293) |
| | | | dimer (minimum) dG = −2.16 | | | | | |
| Set 2 | | 1381-2005 | | | | | GC | |
| label | 5′pos | 3′pos | len | Tm | 5′dG | 3′dG | rate | Sequence |
| F3 | 233 | 257 | 25 | 56.76 | −5.3 | −4.24 | 0.32 | CCTTCCATAAATATTCATGAAGGAT (1294) |
| B3 | 425 | 442 | 18 | 55.77 | −2.39 | −7.2 | 0.44 | AAATTTGATGTGCTCGCG (1295) |
| FIP | | | 47 | | | | | TCAAGTCGTAAAAGTATGGATCCAA-CCTGGAAATATCAAATGATGGT |
| | | | | | | | | (1296) |
| BIP | | | 43 | | | | | CTAGGAACATCGTCGGATGAGA-CAGAAACACCTGTATCATTCA |
| | | | | | | | | (1297) |
| F2 | 259 | 280 | 22 | 55.91 | −5.7 | −4.9 | 0.36 | CCTGGAAATATCAAATGATGGT (1298) |
| F1c | 300 | 324 | 25 | 60.07 | −4.41 | −4.41 | 0.36 | TCAAGTCGTAAAAGTATGGATCCAA (1299) |
| B2 | 393 | 413 | 21 | 55.12 | −4.02 | −4.07 | 0.38 | CAGAAACACCTGTATCATTCA (1300) |
| B1c | 332 | 353 | 22 | 60.75 | −4.27 | −4.15 | 0.5 | CTAGGAACATCGTCGGATGAGA (1301) |
| LB | 359 | 383 | 25 | 60.89 | −4.94 | −3.67 | 0.4 | AGAGGAGATGTTCCGAAATCAATTC (1302) |
| | | | dimer (minimum) dG = −1.69 | | | | | |
| Set 3 | | 1381-2005 | | | | | GC | |
| label | 5′pos | 3′pos | len | Tm | 5′dG | 3′dG | rate | Sequence |
| F3 | 210 | 231 | 22 | 56.36 | −4.86 | −4.32 | 0.36 | TCTTGGAAGAAGGTTATGATGA (1303) |
| B3 | 425 | 442 | 18 | 55.77 | −2.39 | −7.2 | 0.44 | AAATTTGATGTGCTCGCG (1304) |
| FIP | | | 48 | | | | | TCAAGTCGTAAAAGTATGGATCCAA-TAAATATTCATGAAGGATCCCTG |
| | | | | | | | | (1305) |
| BIP | | | 43 | | | | | CTAGGAACATCGTCGGATGAGA-CAGAAACACCTGTATCATTCA |
| | | | | | | | | (1306) |
| F2 | 240 | 262 | 23 | 55.42 | −1.98 | −5.7 | 0.35 | TAAATATTCATGAAGGATCCCTG (1307) |
| F1c | 300 | 324 | 25 | 60.07 | −4.41 | −4.41 | 0.36 | TCAAGTCGTAAAAGTATGGATCCAA (1308) |
| B2 | 393 | 413 | 21 | 55.12 | −4.02 | −4.07 | 0.38 | CAGAAACACCTGTATCATTCA (1309) |
| B1c | 332 | 353 | 22 | 60.75 | −4.27 | −4.15 | 0.5 | CTAGGAACATCGTCGGATGAGA (1310) |
| LB | 359 | 383 | 25 | 60 89 | −4.94 | −3.67 | 0.4 | AGAGGAGATGTTCCGAAATCAATTC (1311) |
| | | | dimer(minimum) dG = −2.36 | | | | | |
| Set 4 | | 1522-1776 | | | | | GC | |
| label | 5′pos | 3′pos | len | Tm | 5′dG | 3′dG | rate | Sequence |
| F3 | 1 | 21 | 21 | 56.35 | −6.3 | −5.08 | 0.38 | GGGACCAATTATTCTTTTGCA (1312) |
| B3 | 191 | 209 | 19 | 56.29 | −5.68 | −4.27 | 0.53 | CATCCGACGATGTTCCTAG (1313) |
| FIP | | | 48 | | | | | GGATCATCATAACCTTCTTCCAAGA-GCTTATTTTGCTTTCACAAATCC |
| | | | | | | | | (1314) |
| BIP | | | 47 | | | | | ATGAAGGATCCCTGGAAATATCAAA-TCTTCAAGTCGTAAAAGTATGG |
| | | | | | | | | (1315) |
| F2 | 23 | 45 | 23 | 57.04 | −3.97 | −4.01 | 0.35 | GCTTATTTTGCTTTCACAAATCC (1316) |
| F1c | 69 | 93 | 25 | 60.23 | −4.76 | −4.86 | 0.4 | GGATCATCATAACCTTCTTCCAAGA (1317) |
| B2 | 165 | 186 | 22 | 55.41 | −4.27 | −4.23 | 0.36 | TCTTCAAGTCGTAAAAGTATGG (1318) |
| B1c | 108 | 132 | 25 | 60.09 | −3.9 | −3.57 | 0.36 | ATGAAGGATCCCTGGAAATATCAAA (1319) |
| | | | dimer (minimum) dG = −2.30 | | | | | |
| Set 5 | | 1522-1776 | | | | | GC | |
| label | 5′pos | 3′pos | len | Tm | 5′dG | 3′dG | rate | Sequence |
| F3 | 38 | 58 | 21 | 55.06 | −3.71 | −4.74 | 0.33 | ACAAATCCCTTAGAAAAAGCT (1320) |
| B3 | 206 | 227 | 22 | 55.62 | −4.25 | −4.25 | 0.41 | CATCTCCTCTTTTCATCTCATC (1321) |
| FIP | | | 49 | | | | | TTCCAGGGATCCTTCATGAATATTT-CCATAAAATTCTTGGAAGAAGGTT |
| | | | | | | | | (1322) |
| BIP | | | 44 | | | | | ACCCTACCATATTTCATCTTGGATC-CGACGATGTTCCTAGGTCA |
| | | | | | | | | (1323) |
| F2 | 60 | 83 | 24 | 57.31 | −3.74 | −4.5 | 0.33 | CCATAAAATTCTTGGAAGAAGGTT (1324) |
| F1c | 100 | 124 | 25 | 60.09 | −4.86 | −2.28 | 0.36 | TTCCAGGGATCCTTCATGAATATTT (1325) |
| B2 | 187 | 205 | 19 | 57.8 | −6.37 | −5.25 | 0.53 | CGACGATGTTCCTAGGTCA (1326) |
| B1c | 141 | 165 | 25 | 60.11 | −4.92 | −4.76 | 0.4 | ACCCTACCATATTTCATCTTGGATC (1327) |

Detection of Amplification Products

Amplification products generated by a method provided herein can be detected by a suitable detection process. Non-limiting examples of methods of detection include electrophoresis, nucleic acid sequencing, mass spectrometry, mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, coded microspheres, template-directed incorporation (TDI), fluorescence polarization, colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), digital PCR, nanopore sequencing, chips, and combinations thereof.

In certain embodiments, amplification products are detected using electrophoresis. Any suitable electrophoresis method, whereby amplified nucleic acids are separated by size, can be used in conjunction with the methods provided herein, which include, but are not limited to, standard electrophoretic techniques and specialized electrophoretic techniques, such as, for example capillary electrophoresis (e.g., Capillary Zone Electrophoresis (CZE), also known as free-solution CE (FSCE), Capillary Isoelectric Focusing (CI EF), Isotachophoresis (ITP), Electrokinetic Chromatography (EKC), Micellar Electrokinetic Capillary Chromatography (MECC OR MEKC), Micro Emulsion Electrokinetic Chromatography (MEEKC), Non-Aqueous Capillary Electrophoresis (NACE), and Capillary Electrochromatography (CEC)).

non-limiting standard electrophoresis example is presented as follows. After running an amplified nucleic acid sample in an agarose or polyacrylamide gel, the gel can be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which can then be compared to the control based on the intensity of the band, thus detecting and quantifying the target sequence of interest. In embodiments, where a plurality of primer pairs is used in an amplification reaction, multiple amplification products of varying size can be detected using electrophoresis.

High Resolution Melting (HRM)

In certain embodiments, nucleic acid is analyzed in the methods provided herein using a high-resolution melting (HRM) endpoint assay. In embodiments, an analysis includes performing a high-resolution melting (HRM) endpoint assay on amplification products (e.g., amplification products generated using primers provided herein). In embodiments, an analysis includes performing a high-resolution melting (HRM) endpoint assay on nucleic acid in a mixture (e.g., a mixture of amplification products generated using a plurality of primer pairs).

High resolution melt or high-resolution melting (HRM) analysis is a technique useful for the detection of mutations, polymorphisms, and epigenetic differences in double-stranded DNA. Typically, amplification (e.g., a polymerase chain reaction (PCR)) is performed prior to HRM analysis to amplify a DNA region in which a mutation or other variant of interest is located. The HRM process involves a precise warming of the amplification product from around 50° C. up to around 95° C. At some point during this process, the melting temperature of the amplicon is reached and the two strands of DNA separate (i.e., melt apart).

The separation of strands can be monitored in real-time (e.g., using a fluorescent dye). Dyes that can be used for HRM include intercalating dyes, which specifically bind to double-stranded DNA and emit fluorescence when bound to DNA. At the start of an HRM analysis there is a high level of fluorescence in the sample because of the billions of copies of the amplicon. However, as the sample is heated up and the two strands of the DNA melt apart, presence of double stranded DNA decreases, and thus the fluorescence is reduced. In certain configurations, an HRM machine has a camera that monitors this process by measuring the fluorescence. The machine can plot the data (e.g., as a graph sometimes referred to as a melt curve), showing the level of fluorescence vs.

temperature.

The melting temperature of an amplification product at which the two DNA strands come apart is a predictable parameter, and typically is dependent on the DNA sequence of the amplicon. When comparing two samples from two different plants containing the same TPS gene, for example, amplification products from both samples should have the same shaped melt curve. However, if one of the plants contains a TPS gene variant, this will alter the temperature at which the DNA strands melt apart. Accordingly, the two melt curves will be different. The difference can be subtle, but because HRM machines typically are capable of monitoring the HRM process in high resolution, it generally is possible to accurately document these changes and therefore identify if a mutation or variant is present or absent.

In certain embodiments, an analysis includes detecting one or more genetic variations (e.g., single nucleotide substitutions) in a TPS gene or paralog thereof according to results obtained from a high-resolution melting (HRM) endpoint assay. In embodiments, an analysis includes detecting two or more genetic variations (e.g., single nucleotide substitutions) in a in a TPS gene or paralog thereof, according to results obtained from a high-resolution melting (HRM) endpoint assay. In certain embodiments, an analysis includes detecting three or more genetic variations (e.g., single nucleotide substitutions) in a TPS gene or paralog thereof, according to results obtained from a high-resolution melting (HRM) endpoint assay. In certain embodiments, an analysis includes detecting four or more genetic variations (e.g., single nucleotide substitutions) in a TPS gene or paralog thereof, according to results obtained from a high-resolution melting (HRM) endpoint assay. In certain embodiments, an analysis includes detecting five or more genetic variations (e.g., single nucleotide substitutions) in a TPS gene or paralog thereof, according to results obtained from a high-resolution melting (HRM) endpoint assay. In certain embodiments, an analysis includes detecting six or more genetic variations (e.g., single nucleotide substitutions) in a TPS gene or paralog thereof, according to results obtained from a high-resolution melting (HRM) endpoint assay. In certain embodiments, an analysis includes detecting seven or more genetic variations (e.g., single nucleotide substitutions) in a TPS gene or paralog thereof, according to results obtained from a high-resolution melting (HRM) endpoint assay. In certain embodiments, an analysis includes detecting eight or more genetic variations (e.g., single nucleotide substitutions) in a TPS gene or paralog thereof, according to results obtained from a high-resolution melting (HRM) endpoint assay. In certain embodiments, an analysis includes detecting nine or more genetic variations (e.g., single nucleotide substitutions) in a TPS gene or paralog thereof, according to results obtained from a high-resolution melting (HRM) endpoint assay. In certain embodiments, an analysis includes detecting ten or more genetic variations (e.g., single nucleotide substitutions) in a TPS gene or paralog thereof, according to results obtained from a high-resolution melting (HRM) endpoint assay.

Nucleic Acid Sequencing

In certain embodiments of the methods provided herein, the nucleic acid is sequenced. In embodiments, amplified subsequences of a TPS gene or a paralog thereof are sequenced by a sequencing process. In embodiments, the sequencing process generates sequence reads (or sequencing reads). In certain embodiments, a method herein comprises determining the sequence of a unique subsequence, such as an exon or a portion thereof, of a TPS gene or a paralog thereof, based on the sequence reads. In certain embodiments, a method provided herein includes determining the TPS gene profile, and/or the TPS gene expression profile, of a plant cultivar based on the sequence reads. In embodiments, the methods provided herein include determining one or TPS gene profiles of one or more plant cultivars based on the sequence reads.

Nucleic acid can be sequenced using any suitable sequencing platform, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In embodiments, a high-throughput sequencing method can be used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In embodiments, MPS sequencing methods utilize a targeted approach, where specific chromosomes, genes or regions of interest are sequenced. For example, a targeted approach can include targeting specific TPS genes, or specific unique subsequences of a TPS gene, for sequencing. In certain embodiments, a non-targeted approach is used where most or all nucleic acids in a sample are sequenced, amplified and/or captured randomly.

Non-limiting examples of sequencing platforms include a sequencing platform provided by Illumina® (e.g., HiSeq™, HiSeq™2000, MiSeq™, Genome Analyzer™, and Genome Analyzer™ II sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLiD sequencing system); Roche (e.g., 454 GS FLX+and/or GS Junior sequencing systems); Helicos True Single Molecule Sequencing; Ion semiconductor-based sequencing (e.g., as developed by Life Technologies), WildFire, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing; or any other suitable sequencing platform. Other sequencing methods that can be used to conduct methods herein include digital PCR, sequencing by hybridization, nanopore sequencing, chromosome-specific sequencing (e.g., using DANSR (digital analysis of selected regions) technology).

In certain embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In embodiments, a sequencing process generates short sequencing reads or “short reads.” In embodiments, the nominal, average, mean or absolute length of short reads sometimes is about 10 continuous nucleotides to about 250 or more contiguous nucleotides. In certain embodiments, the nominal, average, mean or absolute length of short reads sometimes is about 50 continuous nucleotides to about 150 or more contiguous nucleotides.

The length of a sequence read often is associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments, sequence reads are of a mean, median, average or absolute length of about 100 bp to about 200 bp.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid.

In certain embodiments, “obtaining” nucleic acid sequence reads of a sample from a plant and/or “obtaining” nucleic acid sequence reads from one or more amplification products can involve directly sequencing nucleic acid to obtain the sequence information. In some embodiments, “obtaining” can involve receiving sequence information obtained directly from a nucleic acid by another.

In certain embodiments, some or all nucleic acids in a sample are enriched and/or amplified (e.g., non-specifically, or specifically using amplification primers described herein) prior to or during sequencing. In certain embodiments, specific nucleic acid species or subsets in a sample are enriched and/or amplified prior to or during sequencing. In some embodiments, nucleic acid from a pathogen may be enriched and/or amplified prior to or during sequencing, while nucleic acid from a host plant is not enriched and/or amplified prior to or during sequencing. For example, nucleic acid from the genome of the plant cultivar can be enriched and/or amplified prior to or during sequencing, while nucleic acid from the cannabis genome is not enriched and/or amplified prior to or during sequencing. In embodiments, nucleic acids in a sample are not enriched and/or amplified prior to or during sequencing.

In certain embodiments, one nucleic acid sample from one plant is sequenced. In certain embodiments, nucleic acids from each of two or more samples are sequenced, where samples are from one plant or from different plants. In certain embodiments, nucleic acid samples from two or more biological samples are pooled, where each biological sample is from one plant or two or more plants, and the pool is sequenced. In the latter embodiments, a nucleic acid sample from each biological sample often is identified by one or more unique identifiers.

A sequencing method can utilize identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes and use of identifiers can facilitate analyzing a number of samples in each lane A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. For example, primers specific for the unique subsequences in the TPS gene profile of a plant genome can be used for a targeted enrichment, amplification and/or sequencing approach. Any suitable method or combination of methods can be used for enrichment, amplification and/or sequencing of one or more subsets of targeted nucleic acids. In certain embodiments, targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets (e.g., primers provided herein). Sequence specific anchors often can be used as sequence-specific primers.

In embodiments, nucleic acid is sequenced and the sequencing product (e.g., a collection of sequence reads) is processed prior to, or in conjunction with, an analysis of the sequenced nucleic acid. For example, sequence reads can be processed according to one or more of the following: aligning, mapping, filtering, counting, normalizing, weighting, generating a profile, and the like, and combinations thereof. Certain processing steps may be performed in any order and certain processing steps may be repeated.

Solid Supports

Provided herein are solid supports that include the primers provided herein. The primers can directly be attached to the solid support, such as by covalent linkage, or can otherwise be associated with the solid support. For example, the primers can include, in addition to a sequence complementary to a unique subsequence of a TPS gene or paralog thereof in the genome of a plant cultivar of interest, a sequence that is complementary to a nucleic acid sequence that is directly attached to the solid support. The solid supports that include the primers provided herein can be contacted with nucleic acid from a sample obtained from a plant cultivar, under conditions that facilitate hybridization of a primer to a corresponding unique subsequence of a TPS gene or paralog thereof in the genome of a plant cultivar of interest. The resulting hybrids can directly be analyzed, such as by a signal or a label, for the presence or absence of hybridized product containing one or more primers specifically bound to a unique subsequence of a TPS gene in the nucleic acid. Alternately, the resulting hybrids can be subjected to polymerase-based extension reaction conditions using, e.g., one or more labeled nucleotides that can be incorporated into an extension product, thereby identifying, based on the presence or absence of a label in the extension product, whether a TPS gene or paralog thereof is present in the genome of a plant cultivar of interest.

The term “solid support” or “solid phase” as used herein refers to a wide variety of materials including solids, semi-solids, gels, films, membranes, meshes, felts, composites, particles, and the like typically used to sequester molecules, and more specifically refers to an insoluble material with which nucleic acid can be associated. A solid support for use with processes described herein sometimes is selected in part according to size: solid supports having a size smaller than the size a microreactor sometimes are selected. Examples of solid supports for use with processes described herein include, without limitation, beads (e.g., microbeads, nanobeads), particles (e.g., microparticles, nanoparticles) and chips.

The terms “beads” and “particles” as used herein refer to solid supports suitable for associating with biomolecules, and more specifically nucleic acids. Beads may have a regular (e.g., spheroid, ovoid) or irregular shape (e.g., rough, jagged), and sometimes are non-spherical (e.g., angular, multi-sided). Particles or beads having a nominal, average or mean diameter less than the nominal, average, mean or minimum diameter of a microreactor can be utilized. Particles or beads having a nominal, average or mean diameter of about 1 nanometer to about 500 micrometers can be utilized, such as those having a nominal, mean or average diameter, for example, of about 10 nanometers to about 100 micrometers; about 100 nanometers to about 100 micrometers; about 1 micrometer to about 100 micrometers; about 10 micrometers to about 50 micrometers; about 1, 5, 10, 15, 20, 25, 30, 35, 40, 45, 50, 55, 60, 65, 70, 75, 80, 85, 90, 95, 100, 200, 300, 400, 500, 600, 700, 800 or 900 nanometers; or about 1, 5, 10, 15, 20, 25, 30, 35, 40, 45, 50, 55, 60, 65, 70, 75, 80, 85, 90, 95, 100, 200, 300, 400, 500 micrometers.

A bead or particle can be made of virtually any insoluble or solid material. For example, the bead or particle can comprise or consist essentially of silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidenedifluoride (PVDF)) and the like. Beads or particles may be swellable (e.g., polymeric beads such as Wang resin) or non-swellable (e.g., CPG). Commercially available examples of beads include without limitation Wang resin, Merrifield resin and Dynabeads®. Beads may also be made as solid particles or particles that contain internal voids.

The solid supports can be provided in a collection of solid supports. A solid support collection can include two or more different solid support species. The term “solid support species” as used herein refers to a solid support in association with one particular primer or primer pair provided herein, or a combination of different primers or primer pairs. In certain embodiments, a solid support includes about 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 650 or 700 or more primers that specifically bind to unique subsequences of one or more TPS genes or paralogs thereof in one or more plant cultivars of interest. The solid supports (e.g., beads) in the collection of solid supports can be homogeneous (e.g., all are Wang resin beads) or heterogeneous (e.g., some are Wang resin beads, and some are magnetic beads). In certain embodiments, one or more primers selected from among SEQ ID NOS:1-1284 are attached or otherwise associated with a solid support, or a collection of solid supports. In embodiments, one or more primers selected from among those set forth in SEQ ID NOS: 1-1284, or sequences that share 90%, 91%, 92%, 93%, 94%, 95%, 96%, 97%, 98%, 99% or more identity with any of the sequences set forth in SEQ ID NOS: 1-1284, are attached or otherwise associated with a solid support, or a collection of solid supports.

The primers attached to the solid supports generally are single-stranded and are of any type suitable for hybridizing sample nucleic acid (e.g., DNA, RNA, analogs thereof (e.g., peptide nucleic acid (PNA)), chimeras thereof (e.g., a single strand comprises RNA bases and DNA bases) and the like). The primers can be associated with the solid support in any manner suitable for hybridization of the primers to nucleic acid from the plant cultivar. The primers can be in association with a solid support by a covalent linkage or a non-covalent interaction. Non-limiting examples of non-covalent interactions include hydrophobic interactions (e.g., C18 coated solid support and tritylated nucleic acid), polar interactions (e.g., “wetting” association between nucleic acid/polyethylene glycol), pair interactions including without limitation, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA) and the like.

The primers provided herein also can be associated with a solid support by different methodology, which include, without limitation (i) sequentially synthesizing nucleic acid directly on a solid support, and (ii) synthesizing nucleic acid, providing the nucleic acid in solution phase and linking the nucleic acid to a solid support. The primers can be linked covalently at various sites in the nucleic acid to the solid support, such as (i) at a 1′, 2′, 3′, 4′ or 5′ position of a sugar moiety or (ii) a pyrimidine or purine base moiety, of a terminal or non-terminal nucleotide of the nucleic acid, for example. The 5′ terminal nucleotide of the primer can be linked to the solid support, in certain embodiments.

Methods for sequentially synthesizing nucleic acid directly on a solid support are known. For example, the 3′ end of nucleic acid can be linked to the solid support (e.g., phosphoramidite method described in Caruthers, Science 230: 281-286 (1985)) or the 5′ end of the nucleic acid can be linked to the solid support (e.g., Claeboe et al, Nucleic Acids Res. 31(19): 5685-5691 (2003)).

Methods for linking solution phase nucleic acid to a solid support also are known (e.g., U.S. Pat. No. 6,133,436, naming Koster et al. and entitled “Beads bound to a solid support and to nucleic acids” and WO 91/08307, naming Van Ness and entitled “Enhanced capture of target nucleic acid by the use of oligonucleotides covalently attached to polymers”). Examples include, without limitation, thioether linkages (e.g., thiolated nucleic acid); disulfide linkages (e.g., thiol beads, thiolated nucleic acid); amide linkages (e.g., Wang resin, amino-linked nucleic acid); acid labile linkages (e.g., glass beads, tritylated nucleic acid) and the like. Nucleic acid can be linked to a solid support without a linker or with a linker (e.g., S. S. Wong, “Chemistry of Protein Conjugation and Cross-Linking,” CRC Press (1991), and G. T. Hermanson, “Bioconjugate Techniques,” Academic Press (1995). A homo or hetero-biofunctional linker reagent, can be selected, and examples of linkers include without limitation N-succinimidyl(4-iodoacetyl) aminobenzoate (SIAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl)cyclohexane-1-carboxylate (SMCC), 6-hydrazinonicotimide (HYNIC), 3-amino-(2-nitrophenyl)propionic acid and the like.

Nucleic acid can be synthesized using standard methods and equipment, such as the ABI®3900 High Throughput DNA Synthesizer and EXPEDITE®8909 Nucleic Acid Synthesizer, both of which are available from Applied Biosystems (Foster City, Calif.). Analogs and derivatives are described in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing nucleic acids containing such analogs or derivatives are disclosed, for example, in the patent publications cited above and in U.S. Pat. Nos. 5,614,622; 5,739,314; 5,955,599; 5,962,674; 6,117,992; in WO 00/75372 and in related publications. In certain embodiments, analog nucleic acids include inosines, abasic sites, locked nucleic acids, minor groove binders, duplex stabilizers (e.g., acridine, spermidine) and/or other melting temperature modifiers (e.g., target nucleic acid, solid phase nucleic acid, and/or primer nucleic acid may comprise an analog).

The density of solid phase-bound primer molecules per solid support unit (e.g., one bead or one sample location of a chip) can be selected. A maximum density can be selected that allows for hybridization of sample nucleic acid from the plant cultivar to solid phase-bound primers. In certain embodiments, solid phase-bound primer density per solid support unit (e.g., nucleic acid molecules per bead) is about 5 nucleic acids to about 10,000 nucleic acids per solid support. The density of the solid phase-bound primer per unit solid support in some embodiments can be about 5, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 nucleic acids per solid support. In certain embodiments the density of the solid phase-bound primer per unit solid support is about 1 to 1 (e.g., one molecule of solid phase nucleic acid to one bead).

In certain embodiments, the solid supports can include any number of primer species useful for carrying out the analysis methods provided herein. Solid supports having primers attached or otherwise attached thereto can be provided in any convenient form for contacting a sample nucleic acid from a plant cultivar, such as solid or liquid form, for example. In certain embodiments, a solid support can be provided in a liquid form optionally containing one or more other components, which include without limitation one or more buffers or salts. Solid supports of a collection can be provided in one container or can be distributed across multiple containers.

Solid supports can be provided in an array in certain embodiments, or instructions can be provided to arrange solid supports in an array on a substrate. The term “array” as used herein can refer to an arrangement of sample locations (for nucleic acid samples from plant cultivars) on a single two-dimensional solid support, or an arrangement of solid supports across a two-dimensional surface. An array can be of any convenient general shape (e.g., circular, oval, square, rectangular). An array can be referred to as an “X by Y array” for square or rectangular arrays, where the array includes X number of sample locations or solid supports in one dimension and Y number of sample locations or solid supports in a perpendicular dimension. An array can be symmetrical (e.g., a 16 by 16 array) or non-symmetrical (e.g., an 8 by 16 array). An array may include any convenient number of sample locations or solid supports in any suitable arrangement. For example, X or Y independently can be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 in some embodiments.

An array can contain one solid support species or multiple solid support species from a collection. The array can be arranged on any substrate suitable for sequence analysis or manufacture processes described herein. Examples of substrates include without limitation flat substrates, filter substrates, wafer substrates, etched substrates, substrates having multiple wells or pits (e.g., microliter (about 1 microliter, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900 and up to about 999 microliter volume), nanoliter (1 nanoliter, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900 and up to about 999 nanoliter volume), picoliter (1 picoliter, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900 and up to about 999 picoliter volume) wells or pits; wells having filter bottoms), substrates having one or more channels, substrates having one or more electrodes, chips and the like, and combinations thereof. Wells or pits of multiple well and pit substrates can contain one or more solid support units (e.g., each unit being a single bead or particle). Substrates can include a suitable material for conducting sequence analysis or nucleic acid manufacture processes described herein, including without limitation, fiber (e.g., fiber filters), glass (e.g., glass surfaces, fiber optic surfaces), metal (e.g., steel, gold, silver, aluminum, silicon and copper; metal coating), plastic (e.g., polyethylene, polypropylene, polyamide, polyvinylidenedifluoride), silicon and the like. In certain embodiments, the array can be a microarray or a nanoarray. A “nanoarray,” often is an array in which solid support units are separated by about 0.1 nanometers to about 10 micrometers, for example from about 1 nanometer to about 1 micrometer (e.g. about 0.1 nanometers, 0.5, 1, 2, 3, 4, 5, 10, 20, 30, 40, 50, 60, 70, 80, 90, 100, 200, 300, 400, 500, 600, 700, 800, 900 nanometers, 1 micrometer, 2, 3, 4, 5, 6, 7, 8, 9, and up to about 10 micrometers). A “microarray” is an array in which solid support units are separated by more than 1 micrometer. The density of solid support units on arrays often is at least 100/cm², and can be 100/cm² to about 10,000/cm², 100/cm² to about 1,000/cm² or about 150, 200, 300, 400, 500, 600, 700, 800, 900, 1000, 2000, 3000, 4000, 5000, 6000, 7000, 8000, 9000 or 10000 solid support units/cm².

Applications/Uses

The methods provided herein can provide an outcome indicative of one or more characteristics of a plant cultivar, including, but not limited to, a TPS gene profile, a terpene profile, a cannabinoid profile, a flavonoid profile, and the presence of a genetic variation in a TPS gene or paralog thereof.

This information in turn permits identifying and selecting plants of desired genotype or phenotype for agricultural, industrial or medicinal applications based on desired characteristics, such as lineage, resistance or affinity for an organism or condition, or therapeutic activity, and using the selected plants or portions or extracts thereof in methods provided herein, such as methods of breeding, methods of cultivating a crop, and methods of treatment.

The methods of preparing and/or analyzing nucleic acids provided herein, and the primers provided herein for such analysis, permit the identification and select for plants that contain the TPS gene(s) and/or variants of the gene(s) that have desirable characteristics such as desired terpene profiles, ratios of monoterpenes to sesquiterpenes, and/or terpenes that confer agronomic or pathogenesis-related traits (such as insect, pest, mold, mildew, fungus, bacterial, or environmental resistance, as well as attract certain predator or beneficial organisms). The selection can, in embodiments, be used to identify desired parental lines for breeding daughter cultivars that contain desired combinations of these TPS genes or variants of these genes. In addition, the primers provided herein can be used to identify offspring/daughter cultivars that contain the desired gene(s)/variants of these gene(s) from one or both parent cultivars. In addition, the methods of preparing and/or analyzing nucleic acids provided herein, and the primers provided herein for such analysis, can be used for lineage-specific analysis to identify related and distant cultivars to in-breed or out-cross plant cultivars, such as Cannabis cultivars, based on the genetic profiling of unique subsequences (e.g., exons) of TPS genes. Other applications include, but are not limited to:

(1) Using the TPS gene-specific primers provided herein to increase or decrease terpene production, concentration and bio-accumulation in a plant cultivar, such as Cannabis, including, but not limited to the following terpenes:

α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A, Artemisinin

(2) Identify more or less active variants of terpene synthase genes for transgenic experiments including CRISPR, Cre-Lox, and other genetic modification applications to transfer the more or less active variant to another Cannabis cultivar via breeding methods strategies while using the primers provided herein to track the inheritance of that gene variant.

(3) Identify the sub-cellular localization of the TPS genes through identifying the amplicons generated in the methods provided.

(4) Selecting for terpene genes with tissue-specific expression behavior, such as root, flower, stem, or leaf specific terpene synthase genes.

(5) Using the TPS gene-specific primers provided herein in a microassay based presence/absence variation (PAV) identification screening tool, to identify the presence or absence of a TPS gene, including whether a genetic variant is present. In embodiments, a panel of information about several or all TPS genes in a plant cultivar can be obtained, and this information can be related to overall terpene production and accumulation in the plant cultivar.

(6) Using the TPS gene-specific primers provided herein in a cDNA microassay based expression screening tool to identify the level of expression of each gene in the TPS family of a plant cultivar and, in embodiments, relating the level of expression of this panel of genes to overall terpene production and accumulation.

(7) Using the TPS gene-specific primers provided herein to identify and select for gene variants of monoterpene synthases that would deplete the pre-cursor pool of GPP to lower overall cannabinoid and flavonoid concentrations and, in embodiments, breeding these genes into a higher cannabinoid producing cultivar to lower overall cannabinoid content.

(8) Using the TPS gene-specific primers provided herein to identify gene variants of monoterpene synthases that would deplete the pre-cursor pool of GPP to raise the overall cannabinoid and flavinoid concentrations and, in embodiments, breeding this genetic profile into another low cannabinoid producing cultivar to higher overall cannabinoid content using these molecular markers.

(9) Using the TPS gene-specific primers provided herein to select TPS gene combinations that provide specific terpene concentration/production profiles in plants of varying cannabinoid concentration, to decrease the cytotoxicity of the plant extract for medicinal application.

(10) Using the TPS gene-specific primers provided herein, select TPS gene variants that are linked to higher or lower cannabinoid producing cultivars.

In certain aspects, the TPS gene-specific primers provided herein, or subsets thereof, can be used, e.g., in genetic testing and/or amplicon sequencing, to identify plants having a TPS gene profile, TPS gene expression profile, one or more TPS gene variants, a terpene profile, a cannabinoid profile, a flavonoid profile or other characteristics or combinations thereof that impart certain properties to the plant including, but not limited to: pathogen resistance (e.g., insect resistance, fungus resistance), adaptability to regional geographic or environmental features that would make the plant less prone to diseases or predators in a certain region or environment (e.g., resistant to certain diseases or predators at the humidity level in the environment in which the plant is grown), or a desired medicinal use or medical effect. In certain embodiments, the medicinal uses/medical effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects. In embodiments, at least one therapeutic effect is AChEI and in certain embodiments, the analytes are terpenes and the terpenes that are scored include one or more terpenes selected from among alpha pinene, eucalyptol, 3 carene, alpha terpinene, gamma terpinene, cis ocimene, trans ocimene and beta caryophyllene oxide. In certain embodiments, at least one therapeutic effect is analgesic and in embodiments, the analytes are terpenes and the terpenes that are scored comprise one or more terpenes selected from among alpha bisabolol, alpha terpineol, alpha phellandrene and nerolidol.

For example, subsets of these primers can be applied in various specific tests to classify a strain's effect on its user through genetic testing and/or amplicon sequencing. In aspects, sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes can be assigned as imparting one or more desired property to a plant cultivar. For example, sets of 1-10 TPS genes, when present in a Cannabis cultivar, can be characterized as involved in a specific feeling achieved from administration, e.g., by inhalation or ingestion of a product derived from the Cannabis cultivar. For example, the primers could be used for exon-specific genotyping on genomic DNA for a specific subset of genes to identify genotypes that lead to a change in the presence or level of certain terpenes that are known to be associated with medical/physiological effects such as energy, sedation, mental clarity, mental and physical impairments, appetite stimulation or suppression, and/or the other common effects that are associated with products derived from Cannabis or other plant cultivars, or that are known to be associated with pathogen resistance in a Cannabis or other plant cultivar. In aspects, the TPS genes or portions thereof identified by the methods provided herein and used in the methods provided herein include any TPS gene or combinations of TPS genes that produce one or more of: one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

Examples of such breeding methods for a plant cultivar include, but are not limited to those described below (TPS gene nomenclature is characterized in part in Allen et al, PLoS ONE, 14(9):e0222363 (2019), the contents of which are expressly incorporated in their entirety by reference herein):

(a)-A method of breeding a plant that would produce a non-volatile extract to preserve the smell, taste, and aroma of the plant material or an extract thereof by selecting parent cultivars to breed offspring expressing terpene synthase genes that provide such properties, e.g., one or more of the genes designated as follows, or genes that are similar thereto in sequence, structure and/or function, and/or products thereof:
Sesquiterpene Synthases, aka “TPS-a” gene list. Examples include:
   TPS4-like
   TPS9-likel
   TPS9-like2
   TPS50
   TPS18
   TPS14
   TPS7
   TPS4
   TPS32
   TPS9
   TPS20
   TPS8-like
   TPS8
   TPS23
   TPS44
   TPS59
   TPS55
   TPS58
   TPS69

For example, TPS4-likeJL, TPS9-like1JL, TPS9-like2JL, TPS50JL, TPS18JL, TPS14JL, TPS7JL, TPS4JL, TPS32JL, TPS9JL, TPS20JL, TPS8-likeJL, TPS8JL, TPS23JL, TPS44JL, TPS59JL, TPS55JL, TPS58JL, TPS69JL.

(b)-A method of breeding a plant that would produce a volatile smell profile to produce an aromatic and fragrant extract and/or have anti-pathogenic properties by selecting parent cultivars to breed offspring expressing terpene synthase genes that provide such properties, e.g., one or more of the genes designated as follows, or genes that are similar thereto in sequence, structure and/or function, and/or products thereof:
Monoterpene Synthases, aka “TPS-b” gene list. Examples include:
   TPS13-like2
   TPS13
   TPS17
   TPS30
   TPS64
   TPS6-like
   TPS6
   TPS11-like
   TPS51
   TPS30-like
   TPS3
   TPS52
   TPS5
   TPS13-likel
   TPS42
   TPS1
   TPS53
   TPS12
   TPS40
   TPS63
   TPS33
   TPS61
   TPS12-like
   TPS62
   TPS2
   TPS43
   TPS11
   TPS38
   TPS36
   TPS37

For example, TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37JL.

(c)-A method of breeding a plant for the absence of one or more monoterpene synthase (TPS-b) genes that use GPP as a precursor to allow for greater cannabinoid production by selecting parent cultivars to breed to breed offspring not expressing or having reduced expression of terpene synthase genes that interfere with cannabinoid production, e.g., one or more of the genes listed in (b) above, or genes that are similar thereto in sequence, structure and/or function, and/or products thereof.

(d)-A method of breeding a plant that would contain one or more root specifically expressed terpene synthases to increase resistance against pests in the soil and/or respond favorably to beneficial microorganisms in the soil such as beneficial insects, mycorrhizal fungi and beneficial bacteria by selecting parent cultivars to breed offspring expressing terpene synthase genes and/or products that provide such properties, e.g., one or more of the genes designated as follows, or genes that are similar thereto in sequence, structure and/or function, and/or products thereof:
TPS11
TPS49
TPS41
TPS12
TPS11-like
TPS36
TPS6
TPS37
TPS64

For example, TPS11JL, TPS49JL, TPS41JL, TPS12JL, TPS11-likeJL, TPS36JL, TPS6JL, TPS37JL, TPS64JL.

(e)-A method of breeding a plant that would contain one or more predominantly stem specifically expressed terpene synthases to increase resistance against pests that are stem-hosted, e.g., stem-hosted insects, by selecting parent cultivars to breed offspring expressing terpene synthase genes and/or products that provide such properties, e.g., one or more of the one or more of the genes designated as follows, or genes that are similar thereto in sequence, structure and/or function, and/or products thereof:
TPS63
TPS43
TPS6-like
TPS33
TPS24

For example, TPS63JL, TPS43JL, TPS41JL, TPS6-likeJL, TPS33JL, TPS24JL.

The methods listed in (f) through (r) below are for the production of desired terpene product profiles for the indicated applications. Examples of enzymes that can generate all or part of the terpene product profiles for Cannabis are listed (“cs” TPS enzymes). It is understood that one of skill in the art can identify, for any given plant cultivar, TPS enzymes that are similar in sequence, structure and/or function as the indicated Cannabis TPS enzymes and can obtain specialized cultivars having similar terpene product profiles.

(f)-A method of breeding a plant for terpene dominance that changes the smell and/or therapeutic/physiologic effect In the methods listed in (f) through (r) below, it is understood that TPS enzymes that are similar in function to the indicated “Cs” (Cannabis Sativa) enzymes

(f)-A method of breeding a plant for terpene dominance that changes the smell and/or therapeutic/physiologic effect of the plant and/or extract thereof, by selecting parent cultivars to breed offspring expressing terpene synthase genes and/or products that provide such properties, e.g., one or more of the following genes and/or products:

| | Terpene Product to |
|---|---|
| Genes: Major Product (Minor Product) | Breed For: |
| CsTPS9FN | β-caryophyllene |
| CsTPS3FN, csTPS5PK, csTPS5FN, csTPS15CT, | Myrcene |
| csTPS17AK, csTPS23Choc, csTPS30PK | |
| (csTPS32PK, csTPS7PK, csTPS1SK, csTPS2SK) | |
| CsTPS31PK, csTPS37FN (csTPS32PK, | Terpinolene |
| csTPS24Choc, csTPS1SK) | |
| csTPS1SK, csTPS14CT (csTPS5FN, csTPS5PK, | Limonene |
| csTPS7FN, csTPS23Choc, csTPS30PK, | |
| csTPS32PK) | |
| CsTPS25LS (CsTPS32PK) | β-Farnescene |
| csTPS2SK (csTPS1SK, csTPS5FN, csTPS30PK) | Alpha-Pinene |

(g)-A method of breeding a plant for producing an energetic effect when the plant or an extract thereof is ingested and/or vaporized (e.g., as a spray or for inhalation), by selecting parent cultivars to breed offspring expressing or not expressing terpene synthase genes and/or products that result in such properties, e.g., presence or lack thereof of one or more of the following genes and/or products:

| | Terpene Product to |
|---|---|
| Genes: Major Product (Minor Product) | Breed For: |
| CsTPS18VF (CsTPS19BL) | S-linalool |
| CsTPS31PK, csTPS37FN (csTPS32PK, | Terpinolene |
| csTPS24Choc, csTPS1SK) | |
| csTPS6PK, csTPS13PK, and csTPS38FN | β-Ocimene |
| (csTPS7AK) | |
| CsTPS2FN, csTPS5FN (CsTPS32PK | α-Pinene >> β-pinene |
| CsTPS18Choc (CsTPS19BL) | Lack of R-linalool |
| (csTPS5PK, csTPS17AK, csTPS31PK, | Lack of α-terpineol |
| csTPS32PK) | |
| | Lack of fenchol |

(h)-A method of breeding a plant for producing a sedative effect when the plant or an extract thereof is ingested and/or vaporized (e.g., as a spray or for inhalation), by selecting parent cultivars to breed offspring expressing or not expressing terpene synthase genes and/or products that result in such properties, e.g., presence or lack thereof of one or more of the following genes and/or products:

| | Terpene Product to |
|---|---|
| Genes: Major Product (Minor Product) | Breed For: |
| CsTPS31PK, csTPS2SK (csTPS1SK, | β-pinene = α-Pinene |
| csTPS5FN, csTPS5PK, csTPS30PK) | (equal amounts) |
| CsTPS18Choc (CsTPS19BL) | R-linalool |
| csTPS1SK, csTPS14CT (csTPS5FN, | Limonene |
| csTPS5PK, csTPS7FN, csTPS23Choc, | |
| csTPS30PK, csTPS32PK) | |
| csTPS18VF, csTPS19BL, csTPS35LS | Trans-nerolidol |
| (csTPS22PK, csTPS25LS, csTPS32PK) | |
| (csTPS5PK, csTPS17AK, csTPS31PK, | Terpineol |
| csTPS32PK) | |
| CsTPS32PK | Camphene |
| csTPS6PK, csTPS13PK, and | Lack of β-ocimene |
| csTPS38FN (csTPS7AK) | |
| CsTPS18VF (CsTPS19BL) | Lack of S-Linalool |
| CsTPS31PK, csTPS37FN (csTPS32PK, | Lack of Terpinolene |
| csTPS24Choc, csTPS1SK) | |

(i)-A method of breeding a plant that would produce a cognitive-enhancing effect when the plant, or extract thereof, is ingested and/or vaporized by selecting parent cultivars to breed offspring expressing terpene synthase genes and/or products that provide such properties, e.g., one or more of the following genes and/or products:

| | Terpene Product to |
|---|---|
| Genes: Major Product (Minor Product) | Breed For: |
| csTPS2FN, csTPS5FN, and csTPS32PK | α-Pinene >> β-pinene |
| csTPS6PK, csTPS13PK, and | β-ocimene |
| csTPS38FN (csTPS7AK) | |
| | Eucalyptol |

(j)-A method of breeding a plant that would produce an appetite-suppressing effect when the plant, or extract thereof, is ingested and/or vaporized by selecting parent cultivars to breed offspring expressing terpene synthase genes and/or products that provide such properties, e.g., one or more of the following genes and/or products:

| | Terpene Product to |
|---|---|
| Genes: Major Product (Minor Product) | Breed For: |
| CsTPS9FN (csTPS4FN, csTPS22PK) | Humulene |

(k)-A method of breeding a plant that would produce an anti-inflammatory effect when the plant, or extract thereof, is ingested and/or vaporized by selecting parent cultivars to breed offspring expressing terpene synthase genes and/or products that provide such properties, e.g., one or more of the following genes and/or products:

| | Terpene Product to |
|---|---|
| Genes: Major Product (Minor Product) | Breed For: |
| csTPS2SK (csTPS1SK, csTPS5FN, csTPS30PK) | α-Pinene |
| CsTPS9FN (csTPS4FN, csTPS22PK) | Humulene |
| CsTPS9FN | β-caryophyllene |

(I)-A method of breeding a plant that would produce an anxiolytic (anti-anxiety) effect when the plant, or extract thereof, is ingested and/or vaporized by selecting parent cultivars to breed offspring expressing terpene synthase genes and/or products that provide such properties, e.g., one or more of the following genes and/or products:

| | Terpene Product to |
|---|---|
| Genes: Major Product (Minor Product) | Breed For: |
| csTPS2SK (csTPS1SK, csTPS5FN, csTPS30PK) | α-Pinene |
| CsTPS9FN (csTPS4FN, csTPS22PK) | Humulene |
| CsTPS9FN | β-caryophyllene |
| csTPS17AK, csTPS18VF, csTPS18Choc, | Linalool |
| csTPS19BL, csTPS29BC, csTPS35LS | |
| (csTPS31PK) | |
| csTPS18VF, csTPS19BL, csTPS35LS | Nerolidol |
| (csTPS22PK, csTPS25LS, csTPS32PK) | |
| csTPS1SK, csTPS14CT (csTPS5FN, csTPS5PK, | Limonene |
| csTPS7FN, csTPS23Choc, csTPS30PK, | |
| csTPS32PK) | |

(m)-A method of breeding a plant that would produce an antinociceptive effect when the plant, or extract thereof, is ingested and/or vaporized by selecting parent cultivars to breed offspring expressing terpene synthase genes and/or products that provide such properties, e.g., one or more of the following genes and/or products:

| | Terpene Product to |
|---|---|
| Genes: Major Product (Minor Products | Breed For: |
| csTPS5PK (csTPS31PK, csTPS32PK) | α-bisabolol |
| (csTPS5PK, csTPS17AK, csTPS31PK, | α -terpineol |
| csTPS32PK) | |
| csTPS18VF, csTPS19BL, csTPS35LS | trans nerolidol |
| (csTPS22PK, csTPS25LS, csTPS32PK) | |
| (CsTPS2SK, csTPS32PK) | α-phellandrene |
| | Eucalyptol |

(n)-A method of breeding a plant that would produce a body relaxing effect when the plant, or extract thereof, is ingested and/or vaporized by selecting parent cultivars to breed offspring expressing terpene synthase genes and/or products that provide such properties, e.g., one or more of the following genes and/or products:

| Genes: Major Product (Minor Product) | Terpene Product to Breed For: |
|---|---|
| csTPS5PK (csTPS31PK, csTPS32PK) | α-bisabolol |
| (csTPS5PK, csTPS17AK, csTPS31PK, | α -terpineol |
| csTPS32PK) | |
| csTPS18VF, csTPS19BL, csTPS35LS | trans nerolidol |
| (csTPS22PK, csTPS25LS, csTPS32PK) | |
| (csTPS2SK, csTPS32PK) | α-phellandrene |

(o)-A method of breeding a plant that would produce an anti-depressant effect when the plant, or extract thereof, is ingested and/or vaporized by selecting parent cultivars to breed offspring expressing terpene synthase genes and/or products that provide such properties, e.g., one or more of the following genes and/or products:

| Genes: Major Product (Minor Product) | Terpene Product to Breed For: |
|---|---|
| CsTPS31PK, csTPS2SK (csTPSISK, | b-pinene = a-Pinene |
| csTPS5FN, csTPS5PK, csTPS30PK) | |
| csTPS1SK, csTPS14CT (csTPS5FN, | Limonene |
| csTPS5PK, csTPS7FN, csTPS23Choc, | |
| csTPS30PK, csTPS32PK) | |
| csTPS18VF, csTPS19BL, csTPS35LS | nerolidol |
| (csTPS22PK, csTPS25LS, csTPS32PK) | |
| csTPS17AK, csTPS18VF, csTPS18Choc, | Linalool |
| csTPS19BL, csTPS29BC, csTPS35LS | |
| (csTPS31PK) | |

(p)-A method of breeding a plant that contains acetyl cholinesterase-inhibitor (AChEI) terpenes by selecting parent cultivars to breed offspring expressing terpene synthase genes and/or products that provide such properties, e.g., one or more of the following genes and/or products:

| Genes: Major Product (Minor Product) | Terpene Product to Breed For: |
|---|---|
| csTPS2SK (csTPS1SK, csTPS5FN, | α-pinene |
| csTPS30PK) | |
| CsTPS31PK, csTPS37FN (csTPS32PK, | Terpinolene |
| csTPS24Choc, csTPSISK) | |
| csTPS6PK, csTPS13PK, and csTPS38FN | β-ocimene |
| (csTPS7AK) | |
| | 3-Carene |
| csTPS33PK | α- and γ-terpinene |
| (csTPS5FN, csTPS7FN, csTPS30PK) | Sabinene |

(q)-A method of breeding a plant that contain one or more of the Herbivore-induced Plant Volatiles (HIPV) terpene synthases (see, e.g., Booth et al., Plant Physiol., 184(1):130-147 (2020), the contents of which are incorporated in their entirety by reference herein), by selecting parent cultivars to breed offspring expressing terpene synthase genes and/or products that provide such properties, e.g., one or more of the following genes and/or products:

| Genes: Major Product (Minor Product) | Terpene Product to Breed For: |
|---|---|
| csTPS6PK, csTPS13PK, csTPS38FN | Ocimene |
| (csTPS7AK) | |
| csTPS17AK, csTPS18VF, csTPS18Choc, | Linalool |
| csTPS19BL, csTPS29BC, csTPS35LS | |
| (csTPS31PK) | |
| csTPS5PK (csTPS31PK, csTPS32PK) | Bisabolol |
| csTPS18VF, csTPS19BL, csTPS35LS | Nerolidol |
| (csTPS22PK, csTPS25LS, csTPS32PK) | |
| csTPS2SK (csTPS1SK, csTPS5FN, | α-pinene |
| csTPS30PK) | |
| (csTPS2SK, csTPS1SK, csTPS5FN, | β-pinene |
| csTPS5PK, csTPS30PK) | |
| csTPS1SK, csTPS14CT (csTPS5FN, | Limonene |
| csTPS5PK, csTPS7FN, csTPS23Choc, | |
| csTPS30PK, csTPS32PK) | |
| (csTPS5PK, csTPS17AK, csTPS31PK, | α-terpineol |
| csTPS32PK) | |
| (csTPS5PK, csTPS17AK, csTPS31PK, | Y-elemene |
| csTPS32PK) | |
| (csTPS5PK, csTPS31PK, csTPS32PK) | Bergamotene |
| (csTPS8FN, csTPS16CC, csTPS22PK, | Eudesmol |
| csTPS28PK) | |
| csTPS16CC | Germacrene B |
| N/A | Guiaol |
| (csTPS5PK, csTPS17AK, csTPS31PK, | Fenchol |
| csTPS32PK) | |
| N/A | Eugenol |

(r)-A method of breeding a plant that produces, in the plant or in an extract thereof, one or more of the following properties by selecting parent cultivars to breed offspring expressing terpene synthase genes and/or products that provide such properties, e.g., one or more of the following genes and/or gene products:

Antibacterial Properties:

| Genes: Major Product (Minor Product) | Terpene Product to Breed For: |
|---|---|
| CsTPS4FN | Aromadendrene |
| | Carvacrol |
| csTPS9FN | β-Caryophyllene |
| TPS-b and/or other | Eucalyptol (1,8-Cineole) |
| TPS-b and/or other | Fenchol |
| csTPS16CC | Germacrene D |
| CsTPS32PK (CsTPS17AK) | Nerol (cis-Geraniol) |
| TPS-b and/or other | Pulegone |
| (csTPS5FN, csTPS7FN, csTPS30PK) | Sabinene |
| CsTPS32PK (CsTPS17AK) | Geraniol |

Antimicrobial Properties:

| Genes: Major Product (Minor Product) | Terpene Product to Breed For: |
|---|---|
| (CsTPS32PK) | Camphor |
| (csTPS30PK, CsTPS7FN) | Sabinene Hydrate |
| csTPS33PK | Thymol |

Funaicidal Properties:

| Genes: Major Product (Minor Product) | Terpene Product to Breed For: |
|---|---|
| (CsTPS35PK) | Citronellol |
| csTPS33PK | para-Cymene |
| TPS-b and/or other | Pulegone |
| CsTPS32PK | Geraniol |

Herbicidal Properties:

| Genes: Major Product (Minor Product) | Terpene Product to Breed For: |
|---|---|
| CsTPS32PK | Geraniol |
| TPS-b and/or other | Pulegone |
| (CsTPS35PK) | Citronellol |
| TPS-b and/or other | Borneol |
| csTPS33PK | para-Cymene |

Pesticidal Properties:

| Genes: Major Product (Minor Product) | Terpene Product to Breed For: |
|---|---|
| CsTPS4FN - TPS9-like2JL, TPS4-likeJL | Aromadendrene |
| CsTPS5PK (CsTPS32PK - TPS5JL) | α-Bisabolol |
| TPS-a | Cedrol |
| CsTPS18VF, csTPS19BL, csTPS35LS, | Nerolidol |
| (csTPS22PK, csTPS25LS, csTPS32PK) | |
| CsTPS18VF, csTPS19BL, csTPS35LS, | trans-Nerolidol |
| (csTPS22PK, csTPS25LS, csTPS32PK) | |
| TPS-a | Guaiol |

Pheromone: Insect attractant (e.g., for pollination; to attract insects that are predators of other insects or other pathogens that cause plant damage)

| Genes: Major Product (Minor Product) | Terpene Product to Breed For: |
|---|---|
| | endo-Borneol |
| | Isoborneol |
| | 3-Carene |
| | Carveol |
| csTPS16CC | Germacrene B |
| CsTPS21AK | Hedycaryol |
| | Menthol |
| CsTPS18VF, csTPS19BL, csTPS35LS, | cis-Nerolidol |
| (csTPS22PK, csTPS25LS, csTPS32PK) | |
| CsTPS6FN, csTPS13PK, csTPS38FN, | cis-β-Ocimene |
| (csTPS17AK) | |
| CsTPS6FN, csTPS13PK, csTPS38FN, | trans-β-Ocimene |
| (csTPS17AK) | |
| (csTPS30PK, CsTPS7FN) | Sabinene Hydrate |
| csTPS33PK | α-Terpinene |
| csTPS33PK | Thymol |
| CsTPS25LS (CsTPS32PK) | β-Farnesene |
| CsTPS25LS | α-Farnesene |
| CsTPS8FN, (csTPS16CC, csTPS22PK, | γ-Eudesmol |
| CsTPS28PK) | |
| CsTPS4FN | Alloaromadendrene |
| (CsTPS8FN, csTPS4FN) | Valencene |
| TPS-b and/or other | Pulegone |

Expectorant Properties:

| Genes: Major Product (Minor Product) | Terpene Product to Breed For: |
|---|---|
| CsTPS32PK | Camphene |
| CsTPS32PK | Geraniol |
| (csTPS30PK, CsTPS7FN) | Sabinene Hydrate |

Non-irritant properties: (Breed for reduced production or absence of, e.g., one or more of the following terpene products that are irritants, which can be useful, e.g., when breeding plants such as Cannabis for dermatological uses in salves, creams, ointment and transdermal applications)

| Genes: Major Product | Terpene Product to Breed for |
|---|---|
| (Minor Product) | Absence/Reduced Amount: |
| TPS-b and/or other | Borneol |
| | α-Cedrene |
| (CsTPS35PK) | Citronellol |
| csTPS33PK | para-Cymene |
| | Fenchone (Presence - Counterirritant) |

The methods provided herein can, in certain aspects, be used to identify plant genotypes, e.g., Cannabis cultivar genotypes, that produce greater HIPV terpene concentrations in response to a pest or pathogen, or in response to one or more signals emitted by a companion Cannabis cultivar, or in response to one or more signals emitted by a plant cultivar of a species other than Cannabis. It is understood by those of skill in the art that when breeding for certain TPS genes, the presence of a certain gene does not mean that it will always be expressed or produce a measurable product in a flower until it is triggered to express such gene and/or produce a product that is a direct or indirect result of the expression of the gene. This includes, for example, the production of a higher concentration of one or more HIPV terpene concentrations when a threshold pest or pathogen pressure is applied, which then induces the expression of the one or more HIPV terpenes. In addition, variants of the TPS family can have a greater or lesser response to these pest or pathogen pressures, which can be identified by barcoding as provided herein, since the barcode of a TPS gene variant can be indicative of the greater or lesser expression response to a given pest or pathogen.

Similarly, the production of a higher concentration of one or more HIPV terpene concentrations in response to one or more external signals produced by one or more companion Cannabis plants or by one or more companion plants of a species other than Cannabis can be dependent on a threshold signal, which then induces the expression of the one or more HIPV terpenes. In addition, variants of the TPS family can have a greater or lesser response to these external signals, which can be identified by barcoding as provided herein, since the barcode of a TPS gene variant can be indicative of the greater or lesser expression response to a given pest. This also includes, for example, the production of a higher concentration of one or more HIPV terpene concentrations when one or more external signals from a companion Cannabis species or other species of plant is applied, which then induces the expression of the one or more HIPV terpenes. In addition, variants of the TPS family can have a greater or lesser response to these external signals, which can be identified by barcoding as provided herein, since the barcode of a TPS gene variant can be indicative of the greater or lesser expression response to an external signal from a companion plant.

In aspects, provided herein are methods of identifying plant cultivars containing terpene synthase gene profiles that result in the expression of terpenoids associated with oviposition deterrence (deter an insect that is a pest from laying eggs on the plant), fumigant insect repellent activity, contact toxicity and/or insect herbivore predator attractant. In certain aspects, the insect oviposition deterrent is selected from among one or more of linalool, α-bisabolol, and trans-neridol or any combinations thereof. In aspects, the contact insecticide is guaiol. In aspects, the fumigant insect repellent is selected from among β-ocimene, α-bisabolol or a combination thereof.

In certain aspects, provided herein are methods of breeding plants that produce terpenoids associated with oviposition deterrence, insect fumigant activity, contact toxicity and/or an insect herbivore predator attractant. In aspects, the breeding comprises creating a plant with an oviposition deterrence profile by crossing a plant that produces an amount of trans-nerolidiol that is at or above a threshold amount with a plant that produces an amount of α-bisabolol that is at or above a threshold amount. In aspects, the breeding comprises creating a plant with an insect fumigant terpene profile by crossing a plant that produces an amount of α-bisabolol that is at or above a threshold amount with a plant that produces β-ocimene in an amount that is at or above a threshold amount. In certain aspects, the breeding comprises creating a plant with both oviposition deterrence and insect fumigant terpene profiles by crossing a plant that produces an amount of α-bisabolol and β-ocimene that is at or above a threshold amount with a plant that produces trans-nerolidiol in an amount that is at or above a threshold amount.

In certain aspects, the methods provided herein can be used to amplify the entire coding sequence of a TPS gene for analyzing, e.g., its homology to other TPS gene sequences by sequencing and/or restriction digest analysis, its terpene production (e.g., in vitro) and/or for transgenic cloning to functionally characterize the gene and/or to create variant cultivars having a desired terpene synthase gene expression profile. For example, amplicons of a TPS gene can be generated using a forward primer closest to the 5′ end of the gene, and a reverse primer closest to the 3′ end of the gene that could amplify the full transcript gene sequence from a given plant cultivar's gDNA, or cDNA library. The resulting amplicons could be subject to any genotyping application such as HRM, sequencing, microarray analysis, restriction enzyme digestion, or other common genotyping methods.

In aspects, the TPS gene of interest can be inserted into a cloning vector for expression in a compatible host cell. A large number of vector-host systems known in the art can be used. Possible vectors include, but are not limited to, plasmids or modified viruses. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector exemplified herein (see e.g., SEQ ID NOS:4 and 5). The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). Prokaryotic and eukaryotic host cells can be used to express a gene contained in a vector. Such cells include bacterial cells, yeast cells, fungal cells, Archea, plant cells, insect cells and animal cells. These include but are not limited to mammalian cell systems infected with virus (e.g., vaccinia virus, adenovirus and other viruses); insect cell systems infected with virus (e.g., baculovirus); microorganisms such as yeast containing yeast vectors; or bacteria transformed with bacteriophage, DNA, plasmid DNA, or cosmid DNA.

The host cells are used to produce a protein encoded by the TPS gene or by a vector containing the gene by growing them under conditions whereby the encoded protein is expressed by the cell. The encoded TPS enzyme can be studied/manipualted in the host cell, or can be recovered from the cell by proteoin isolation and purification methods known to those of skill in the art. In aspects, the host-nucleic acid (vector or gene) system can be enginnered, by methods known to those of skill in the art, to secrete the TPS enzyme into the medium.

In aspects, the TPS gene is involved in the production of terpinolene. In aspects, the primers are as shown below:

Terpinolene - csTPS37FN full mRNA cloning primers (including signal peptide sequences)

| | | Template | | | | | | Self | Self 3′ |
|---|---|---|---|---|---|---|---|---|---|
| | Sequence (5′->3′) | strand | Length | Start | Stop | Tm | GC % | complementarity | complementarity |
| Forward | ATGCAGTGCATGGCT | Plus | 19 | 1 | 19 | 57.17 | 47.37 | 9.00 | 0.00 |
| primer | TTTC (SEQ ID NO: 1328) | | | | | | | | |
| Reverse | TTACATGGGAATAGG | Minus | 30 | 1920 | 1891 | 58.38 | 30.00 | 5.00 | 3.00 |
| primer | GTTAATAATCAAATC | | | | | | | | |
| | (SEQ ID NO:1329) | | | | | | | | |
| Product | 1920 | | | | | | | | |
| length | | | | | | | | | |

In certain aspects, the TPS genes are selected from among the following: 1) TPS9 LPA4 type, 2) TPS9 LPA21.3 type, 3) TPS37 Cleaved (lacking the signal peptide, i.e., does not encode the chloroplast import signal), 4) TPS16CC, and 5) TPS20CT, and the primers are as follows:

Beta-Caryophyllene and Humulene - TPS9L21 (TPS9 LPA21.3)

| | | Template | | | | | | Self | Self 3′ |
|---|---|---|---|---|---|---|---|---|---|
| | Sequence (5'->3') | strand | Length | Start | Stop | Tm | GC % | complementarity | complementarity |
| Forward | AT GTC ATA TCA AGT | Plus | 20 | 1 | 20 | 48.75 | 30.00 | 5.00 | 2.00 |
| primer | TTT AGC (SEQ ID | | | | | | | | |
| | NO: 1330) | | | | | | | | |
| Reverse | TGG GAT TTG ATC | Minus | 23 | 1668 | 1646 | 53.76 | 34.78 | 4.00 | 2.00 |
| primer | TAT AAG TAA CG | | | | | | | | |
| | (SEQ ID NO: 1331) | | | | | | | | |
| Product | 1668 | | | | | | | | |
| length | | | | | | | | | |

Beta-Caryophyllene and Humulene - TPS9L4 (TPS9 LPA4)

| | | Template | | | | | | Self | Self 3′ |
|---|---|---|---|---|---|---|---|---|---|
| | Sequence (5′->3′) | strand | Length | Start | Stop | Tm | GC % | complementarity | complementarity |
| Forward | AT GTC ATA TCA AGT | Plus | 20 | 1 | 20 | 48.75 | 30.00 | 5.00 | 2.00 |
| primer | TTT AGC (SEQ ID | | | | | | | | |
| | NO: 1330) | | | | | | | | |
| Reverse | CGG GAT TTG ATC | Minus | 23 | 1668 | 1646 | 54.82 | 39.13 | 4.00 | 2.00 |
| primer | TAT AAG TAA CG (SEQ | | | | | | | | |
| | ID NO: 1332) | | | | | | | | |
| Product | 1668 | | | | | | | | |
| length | | | | | | | | | |

Germacrene B/gamma-eudesmol - TPS1600

| | | Template | | | | | | Self | Self 3′ |
|---|---|---|---|---|---|---|---|---|---|
| | Sequence (5′->3′) | strand | Length | Start | Stop | Tm | GC % | complementarity | complementarity |
| Forward | AT GTC TAG TCA AGT | Plus | 20 | 1 | 20 | 52.37 | 40.00 | 6.00 | 2.00 |
| primer | GTT AGC (SEQ ID | | | | | | | | |
| | NO: 1333) | | | | | | | | |
| Reverse | TAA TGG GAT GGG | Minus | 22 | 1713 | 1692 | 54.59 | 40.91 | 4.00 | 2.00 |
| primer | ATC TAT AAG C (SEQ | | | | | | | | |
| | ID NO: 1334) | | | | | | | | |
| Product | 1713 | | | | | | | | |
| length | | | | | | | | | |

Hedycaryol - CsTPS200T

| | | Template | | | | | | Self | Self 3′ |
|---|---|---|---|---|---|---|---|---|---|
| | Sequence (5′->3′) | strand | Length | Start | Stop | Tm | GC % | complementarity | complementarity |
| Forward | AT GTC AAA TAT TCA | Plus | 23 | 1 | 23 | 52.90 | 30.43 | 6.00 | 2.00 |
| primer | AGT CTT AGC | | | | | | | | |
| | (SEQ ID NO: 1335) | | | | | | | | |
| Reverse | TAA TGG GAT GGG | Minus | 26 | 1653 | 1628 | 55.68 | 30.77 | 7.00 | 0.00 |
| primer | ATC TAT AAG C (SEQ | | | | | | | | |
| | ID NO: 1336) | | | | | | | | |
| Product | 1653 | | | | | | | | |
| length | | | | | | | | | |

Terpinolene - csTPS37FN coding primers (cleaved; does not include signal peptide sequences)

| | | Template | | | | | | Self | Self 3′ |
|---|---|---|---|---|---|---|---|---|---|
| | Sequence (5′->3′) | strand | Length | Start | Stop | Tm | GC % | complementarity | complementarity |
| Forward | ACT GTG GTC GAT | Plus | 23 | 184 | 206 | 59.55 | 47.83 | 4.00 | 0.00 |
| primer | AAC CCT AGT TC | | | | | | | | |
| | (SEQ ID NO: 1337) | | | | | | | | |
| Reverse | CAT GGG AAT AGG | Minus | 27 | 1917 | 1891 | 56.63 | | 33.33 | 5.00 |
| primer | GTT AAT AAT CAA | | | | | | | | |
| | ATC (SEQ ID NO: | | | | | | | | |
| | 1338) | | | | | | | | |
| Product | 1734 | | | | | | | | |
| length | | | | | | | | | |

In embodiments, the above-mentioned primers can be adapted for use in commercial cloning, expression and/or amplification kits. For example, if a directional cloning/amplification/expression system is performed, such as TOPO directional cloning/amplification/expression (Thermofisher Scientific, USA), using one or more of the above-mentioned sets of primers, a 5′ CACC 3′ sequence can be attached on the 5′ end of any of the forward primera to allow for an overhang to be created during the process of PCR, which then allows for subsequent cloning/amplification/expression protocols to be carried out. In embodiments, the 5′CACC3′ tag can be interchanged with overhang sequences designated by kits other than the TOPO kit, to accomplish the same goal of functional characterization. In embodiments, introduction of an artificial start codon (ATG) at the 5′ end of a forward primer can permit the coding mRNA sequence to be expressed and properly folded in a bacterial or non-eukaryotic expression system. In embodiments, the ATG start codon is added to the 5′ end of the primer of SEQ ID NO:1337.

Use of Devices, Programs and Media

In certain embodiments, an outcome and/or classification obtained by the methods provided herein is provided using a suitable visual medium (e.g., a component of a machine, e.g., a printer or display). A classification and/or outcome may be provided in the form of a report. A report typically includes a display of an outcome and/or classification (e.g., a value, one or more characteristics of a sample, an assessment or probability of presence or absence of a genotype, phenotype or genetic variation; and/or an assessment or probability of a genotype, genetic variation, and/or genetic variation signature, e.g., of a TPS gene profile for a plant cultivar), sometimes includes an associated confidence parameter, and sometimes includes a measure of performance for a test used to generate the outcome and/or classification. A report sometimes includes a recommendation for a follow-up test (e.g., a test that confirms the outcome or classification).

A report can be displayed in a suitable format that facilitates determination of presence or absence of a genotype, phenotype, genetic variation or genetic variation signature. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing. In embodiments, the report can be in the form of a barcode, where each line/number in the barcode represents a TPS gene or paralog thereof that is present in the plant cultivar.

A report can be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, or any other medium described in the previous paragraph. A laboratory file may be in tangible form or electronic form (e.g., computer readable form), in certain embodiments. After a report is generated and transmitted, a report can be received by obtaining, via a suitable communication medium, a written and/or graphical representation of an outcome and/or classification, which upon review allows a qualified individual to make a determination as to one or more characteristics of a sample from a plant cultivar, such as the presence or absence of a genotype, phenotype or genetic variation in a test sample (e.g., a Cannabis plant sample).

An outcome and/or classification can be provided by and obtained from a laboratory (e.g., obtained from a laboratory file). A laboratory file can be generated by a laboratory that carries out one or more tests for determining one or more characteristics of a sample such as presence or absence of a genotype, phenotype or genetic variation for a test sample (e.g., a Cannabis plant sample). Laboratory personnel (e.g., a laboratory manager) can analyze information associated with test samples (e.g., test profiles, reference profiles, test values, reference values, level of deviation) underlying an outcome and/or classification. For calls pertaining to presence or absence of a genotype, phenotype or genetic variation that are close or questionable, laboratory personnel can re-run the same procedure using the same (e.g., aliquot of the same sample) or different test sample from a plant.

A laboratory can be in the same location or different location (e.g., in another town, city or country) as personnel assessing the presence or absence of a genotype, phenotype or genetic variation from the laboratory file. For example, a laboratory file can be generated in one location and transmitted to another location in which the information for a test sample therein is assessed by a qualified individual, and optionally, transmitted to the facility and/or grower from which the test sample was obtained. A laboratory sometimes generates and/or transmits a laboratory report containing a classification of presence or absence of a genotype, phenotype or a genetic variation for a test sample (e.g., a Cannabis plant sample).

Machines, Software and Interfaces

Methods described herein (e.g., processing amplification results, processing high resolution melting (HRM) assay results, processing sequence read data, determining one or more characteristics of a plant cultivar based on sequence read data, associating one or more phenotypes of a plant cultivar (e.g., terpene, cannabinoid or flavonoid production profiles with one or more genotypes or genetic variants of the plant cultivar, and/or providing an outcome (e.g., indicated as desirable for breeding, or cultivating as a crop, or for therapeutic use, based on the specified selection criteria) can be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., processing amplification results, processing high resolution melting (HRM) assay results, processing sequence read data, determining one or more characteristics of a plant cultivar based on sequence read data, associating one or more phenotypes of a plant cultivar (e.g., a Cannabis plant) with one or more genotypes or genetic variations for the plant cultivar and/or providing an outcome are performed by automated methods. In embodiments, one or more steps of a method described herein are carried out by a microprocessor and/or computer, and/or carried out in conjunction with memory. In certain embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces can be used to conduct methods described herein. Using machines, software and interfaces, a user can enter, request, query or determine options for using particular information, programs or processes (e.g., processing amplification results, processing high resolution melting (HRM) assay results, processing sequence read data, determining one or more characteristics of a plant cultivar based on sequence read data, associating one or more phenotypes of a plant cultivar with one or more genotypes or genetic variations and/or providing an outcome, which can involve implementing statistical analysis algorithms, statistical significance algorithms, statistical algorithms, iterative steps, validation algorithms, and graphical representations, for example. In embodiments, a data set can be entered by a user as input information, a user may download one or more data sets by suitable hardware media (e.g., flash drive), and/or a user can send a data set from one system to another for subsequent processing and/or providing an outcome (e.g., send sequence read data from a sequencer to a computer system for sequence read processing; send processed sequence read data to a computer system for further processing and/or yielding an outcome and/or report).

A system typically includes one or more machines. Each machine includes one or more of memory, one or more microprocessors, and instructions. Where a system includes two or more machines, some or all of the machines can be located at the same location, some or all of the machines can be located at different locations, all of the machines can be located at one location and/or all of the machines may be located at different locations. Where a system includes two or more machines, some or all of the machines can be located at the same location as a user, some or all of the machines can be located at a location different than a user, all of the machines can be located at the same location as the user, and/or all of the machine can be located at one or more locations different than the user.

A system sometimes includes a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine an outcome from the sequence reads.

A user can, for example, place a query to software which then may acquire a data set via internet access, and in certain embodiments, a programmable microprocessor may be prompted to acquire a suitable data set based on given parameters. A programmable microprocessor also can prompt a user to select one or more data set options selected by the microprocessor based on given parameters. A programmable microprocessor can prompt a user to select one or more data set options selected by the microprocessor based on information found via the internet, other internal or external information, or the like. Options can be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein can include general components of computer systems, such as, for example, network servers, laptop systems, desktop systems, handheld systems, personal digital assistants, computing kiosks, and the like. A computer system can include one or more input means such as a keyboard, touch screen, mouse, voice recognition or other means to allow the user to enter data into the system. A system can further include one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components can be connected to a central processing unit which may comprise among other components, a microprocessor for executing program instructions and memory for storing program code and data. In embodiments, processes can be implemented as a single user system located in a single geographical site. In certain embodiments, processes can be implemented as a multi-user system. In the case of a multi-user implementation, multiple central processing units can be connected by means of a network. The network can be local, encompassing a single department in one portion of a building, an entire building, span multiple buildings, span a region, span an entire country or be worldwide. The network can be private, being owned and controlled by a provider, or it can be implemented as an internet-based service where the user accesses a web page to enter and retrieve information. Accordingly, in certain embodiments, a system includes one or more machines, which can be local or remote with respect to a user. More than one machine in one location or multiple locations can be accessed by a user, and data can be mapped and/or processed in series and/or in parallel. Thus, a suitable configuration and control can be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface can be used to receive signal information that can be detected by a signal detection module.

Data can be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

A system can include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can include a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules can be located in different machines in different physical locations.

A machine, in some embodiments, includes at least one microprocessor for carrying out the instructions in a module. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In embodiments, a machine includes a module one or more modules. A machine that includes a module often is capable of receiving and transferring one or more of data and/or information to and from other modules.

In certain embodiments, a machine includes peripherals and/or components. In certain embodiments, a machine can include one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, VViFi, and the like), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users can access a remote system maintained by an organization to remotely access software. Software can obtain or receive input information. Software can include a module that specifically obtains or receives data and may include a module that specifically processes the data (e.g., a processing module that processes received data). The terms “obtaining” and “receiving” input information refers to receiving data by computer communication means from a local, or remote site, human data entry, or any other method of receiving data. The input information may be generated in the same location at which it is received, or it may be generated in a different location and transmitted to the receiving location. In embodiments, input information is modified before it is processed (e.g., placed into a format amenable to processing, e.g., tabulated).

Software can include one or more algorithms in certain embodiments. An algorithm can be used for processing data and/or providing an outcome or report according to a finite sequence of instructions. An algorithm often is a list of defined instructions for completing a task. Starting from an initial state, the instructions can describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In embodiments, an algorithm can be configured or modified to include margin of errors, statistical analysis, statistical significance, and/or comparison to other information or data sets (e.g., applicable when using a neural net or clustering algorithm).

In certain embodiments, several algorithms can be implemented for use in software. These algorithms can be trained with raw data in some embodiments. For each new raw data sample, the trained algorithms can produce a representative processed data set or outcome. A processed data set sometimes is of reduced complexity compared to the parent data set that was processed. Based on a processed set, the performance of a trained algorithm can be assessed based on sensitivity and specificity, in some embodiments. An algorithm with the highest sensitivity and/or specificity can be identified and utilized, in certain embodiments.

In certain embodiments, simulated (or simulation) data can aid data processing, for example, by training an algorithm or testing an algorithm. In embodiments, simulated data includes hypothetical various samplings of different groupings of sequence reads, genotypes, phenotypes, genetic variations, and/or genetic variation signatures. Simulated data can be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In embodiments, an empirical model may be assessed, in which it is assumed that at least one sample matches a reference sample (with or without resolved variations). In certain embodiments, another distribution, such as a Poisson distribution for example, can be used to define the probability distribution.

A system can include one or more microprocessors in certain embodiments. A microprocessor can be connected to a communication bus. A computer system can include a main memory, often random access memory (RAM), and can also include a secondary memory. Memory in some embodiments includes a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor can implement software in a system. In some embodiments, a microprocessor can be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample.

In certain embodiments, secondary memory can include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

Compositions and Kits

Provided in certain embodiments are compositions. Compositions useful for carrying out any of the methods described herein are provided. For example, compositions that include any of the primers, primer pairs and sets of more than one primer pair described herein are provided. In certain embodiments, the compositions include one or more of primers or primer pairs for identifying monoterpene synthases, primers or primer pairs for identifying diterpene synthases and primers or primer pairs for identifying sesquiterpene synthases. In embodiments, the compositions include one or more of primers or primer pairs for identifying monoterpene synthases. In embodiments the compositions include one or more of primers or primer pairs for identifying diterpene synthases. In embodiments, the compositions include one or more of primers or primer pairs for identifying sesquiterpene synthases. In any of the compositions provided herein, in certain embodiments, the primers are selected from among those of SEQ ID NOS:1-1284; from among the LAMP primers of SEQ ID NOS:1285-1327 or the LAMP primer sets of SEQ ID NOS:1285-1293; 1294-1302; 1303-1311; 1312-1319 and 1320-1327; or from among the full-length amplifying primers of SEQ ID NOS:1328-1338. In embodiments, the primers are selected from among those set forth in SEQ ID NOS: 1-1284, from among the LAMP primers of SEQ ID NOS:1285-1327 or the LAMP primer sets of SEQ ID NOS:1285-1293; 1294-1302; 1303-1311; 1312-1319 and 1320-1327, or from among the full-length amplifying primers of SEQ ID NOS:1328-1338 or sequences that share 90%, 91%, 92%, 93%, 94%, 95%, 96%, 97%, 98%, 99% or more identity with any of the sequences set forth in SEQ ID NOS: 1-1284, the LAMP primers of SEQ ID NOS:1-1327 or the LAMP primer sets of SEQ ID NOS:1285-1293; 1294-1302; 1303-1311; 1312-1319 and 1320-1327, or from among the full-length amplifying primers of SEQ ID NOS:1328-1338. In embodiments, any of the forward primers in the primer pairs provided in SEQ ID NOS: 1-1284, or in sequences that share 90%, 91%, 92%, 93%, 94%, 95%, 96%, 97%, 98%, 99% or more identity with any of the sequences set forth in SEQ ID NOS: 1-1284, can be paired with any of the reverse primers of the primer pairs having the sequences set forth in SEQ ID NOS: 1-1284, or in sequences that share 90%, 91%, 92%, 93%, 94%, 95%, 96%, 97%, 98%, 99% or more identity with any of the sequences set forth in SEQ ID NOS: 1-1284.

In embodiments, the primer pairs of the compositions provided herein are complementary to a unique subsequence of a TPS gene or a paralog thereof, wherein the unique subsequence of the TPS gene or paralog thereof is different than the other subsequences of the TPS gene or paralog thereof and is different than the subsequences of other TPS genes or paralogs thereof. In certain embodiments, the TPS gene or a paralog thereof is of a Cannabis cultivar. In embodiments, the compositions provided herein include at least one primer or primer pair that is complementary to a genetically modified TPS gene or paralog thereof.

Kits

Provided in certain embodiments are kits. The kits can include any components and compositions described herein, e.g., primers, primer pairs, primer sets, including LAMP primer sets, reagents for hybridization or amplification of at least one TPS gene or paralog thereof, solid supports, collections of solid supports, one or more detection labels for detecting amplicons and instructions for use to, e.g., analyze the TPS gene profile of a plant cultivar of interest, or to identify a genetically modified plant cultivar of interest. A kit for amplifying nucleic acid from an RNA template can further include reagents for reverse transcription (e.g., for generating cDNA).

Components of a kit can be present in separate containers, or multiple components can be present in a single container. In embodiments, primers are provided such that each container contains a single primer pair (e.g., for individual amplification reactions). In certain embodiments, primers are provided such that one container contains a plurality of primer pairs (e.g., for multiplexed amplification reactions). Suitable containers include a single tube (e.g., vial), one or more wells of a plate (e.g., a 96-well plate, a 384-well plate, and the like), chips and the like.

Kits also can include instructions for performing one or more methods described herein and/or a description of one or more components described herein. For example, a kit can include instructions for using the amplification primers provided herein, to amplify nucleic acid (e.g., to amplify unique subsequences of a TPS gene or paralog thereof in a plant cultivar). In certain embodiments, a kit can include instructions or a guide for interpreting the results of an amplification reaction. Instructions and/or descriptions can be in printed form and can be included in a kit insert. In embodiments, instructions and/or descriptions are provided as an electronic storage data file present on a suitable computer readable storage medium, e.g., portable flash drive, DVD, CD-ROM, diskette, and the like. A kit also can include a written description of an internet location that provides such instructions or descriptions.

### EXAMPLES

The examples set forth below illustrate certain embodiments and do not limit the technology.

### Example 1

Primer Design to Identify Terpene Synthase Genes Based on Exon-Specific Amplification

This example describes the design and synthesis of primers that differentiate between terpene synthase genes or paralogs thereof based on amplifying unique regions encompassing all or a portion of certain exons of each gene or paralog thereof.

Primers were designed to target unique sequences within each terpene synthase exon of 75 terpene synthase genes, based on the genome assembly and sequence of Cannabis sativa (eudicots) (Genbank Accession Number GCF_900626175.1). The high homology of terpene synthase exon paralogs throughout the genome posed a challenge with respect to obtaining primers that target single specific target sequences and satisfy primer design parameters. 74 out of the 75 genes were able to be uniquely targeted, the exception being the Cannabis sativa TPS2 gene from the Jamaican Lion cultivar (CsTPS2JL). The parameters were as follows, with exceptions where specified if the ideal design was not possible due to the sequence similarity between similar exons in the genome.

Tools Used: NCBI's primer designer tool using Primer3

Ideal Target: Primers target conserved regions of the exon that contains a region of variability within the amplicon but not in the primed loci, i.e., in the primer binding sites.

Ideal product size: 50-150 bp

Maximum allowed product size: 292 bp

Primer Melting Temperature: 57-63° C. (max Tm difference 3° C.)

Must have 5 mismatches overall, to non-specific genomic targets

In addition, for non-specific genomic targets, must have 3 mismatches within 5 bp of the 3′ end of each primer

When possible, primers were designed to have a GC clamp on the 3′ and 5′ ends

Maximum Self Complementarity: Generally 5, with 6 permitted in a few primer sets Maximum 3′ Self Complementarity: 5

Each primer set that was designed aimed to span as much of an entire individual exon target as possible, or multiple sets of primers were designed for an individual exon target to cover as much of the exon target as specificity would allow. This allows for each individual exon to be genotyped, providing an exon-based enzyme genotyping system that can be used on genomic DNA and provides a higher level of differentiation between genes that have a high percentage of sequence identity.

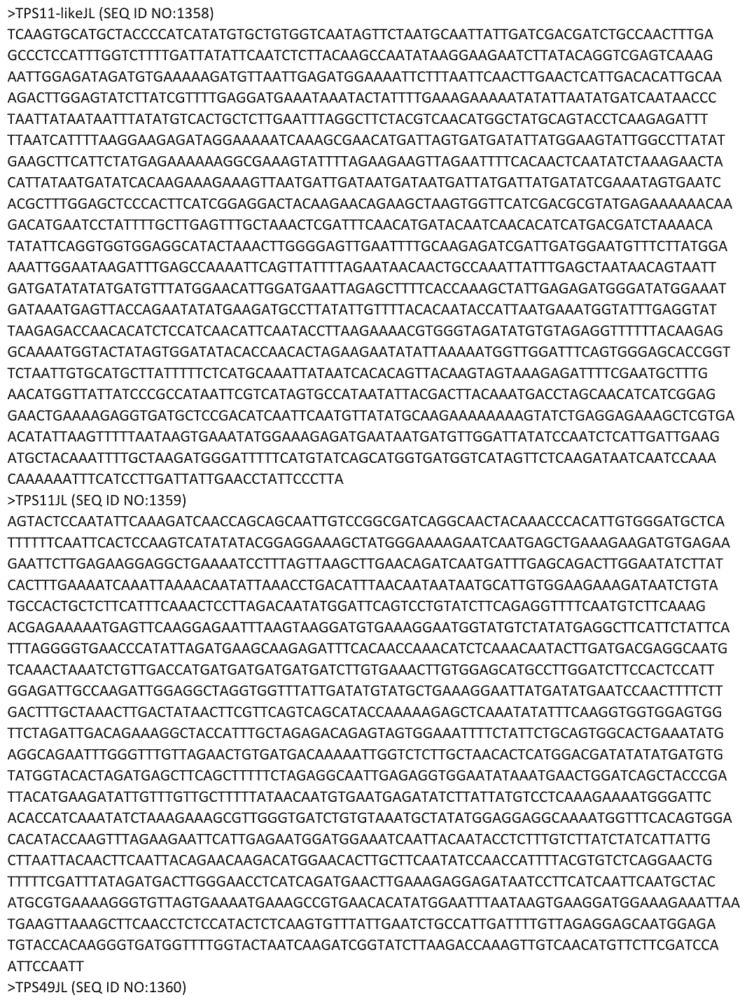

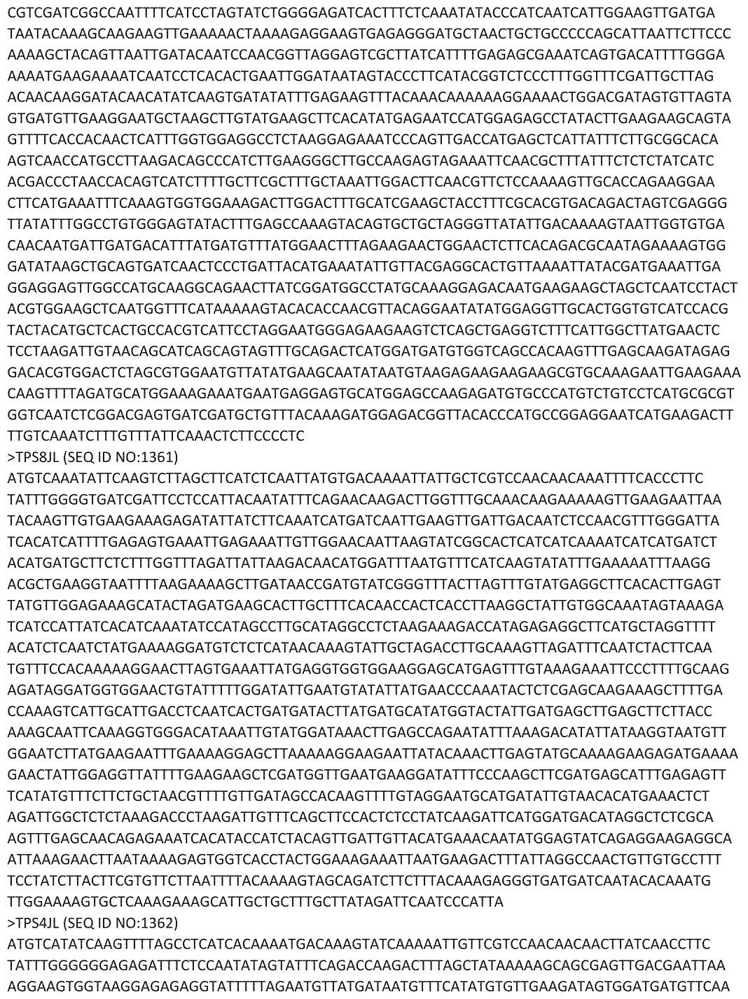

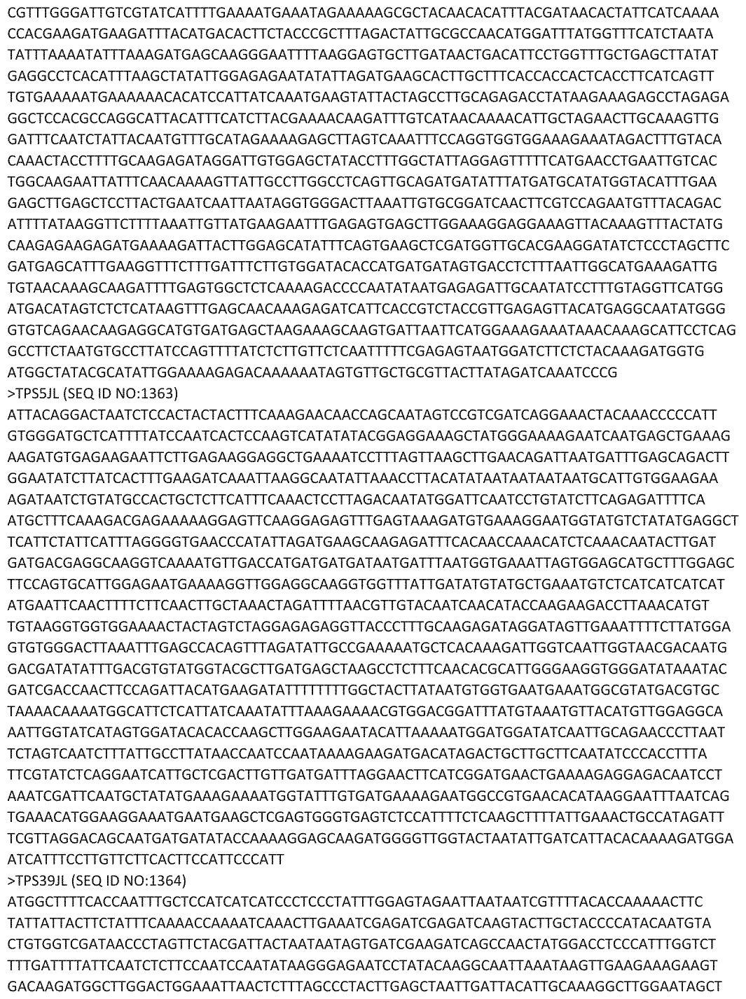

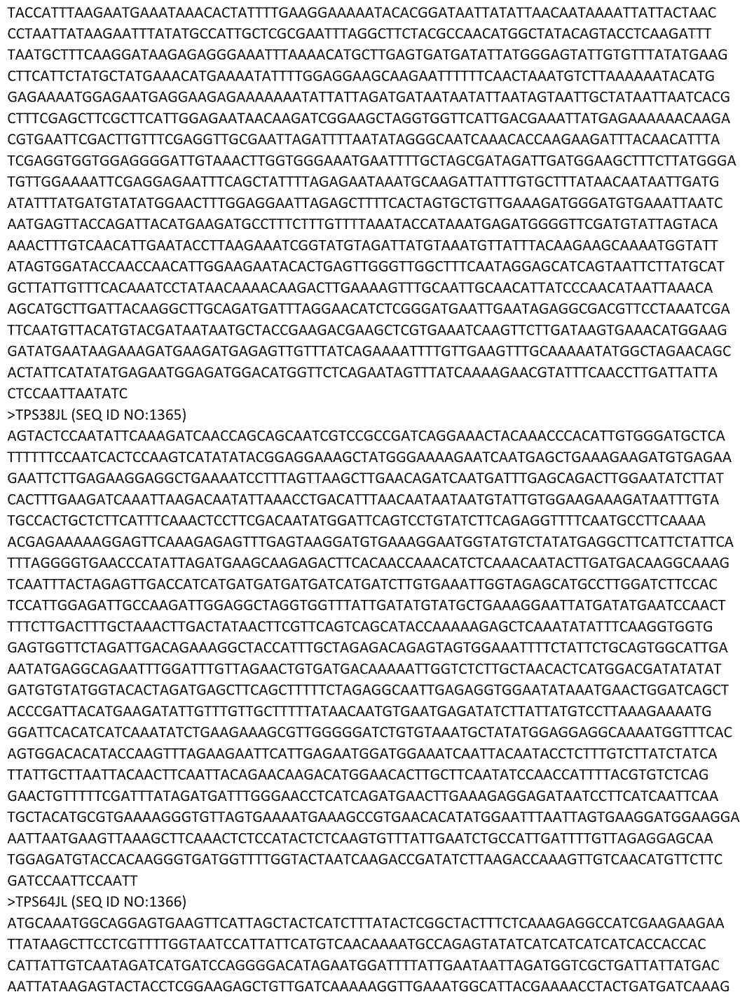

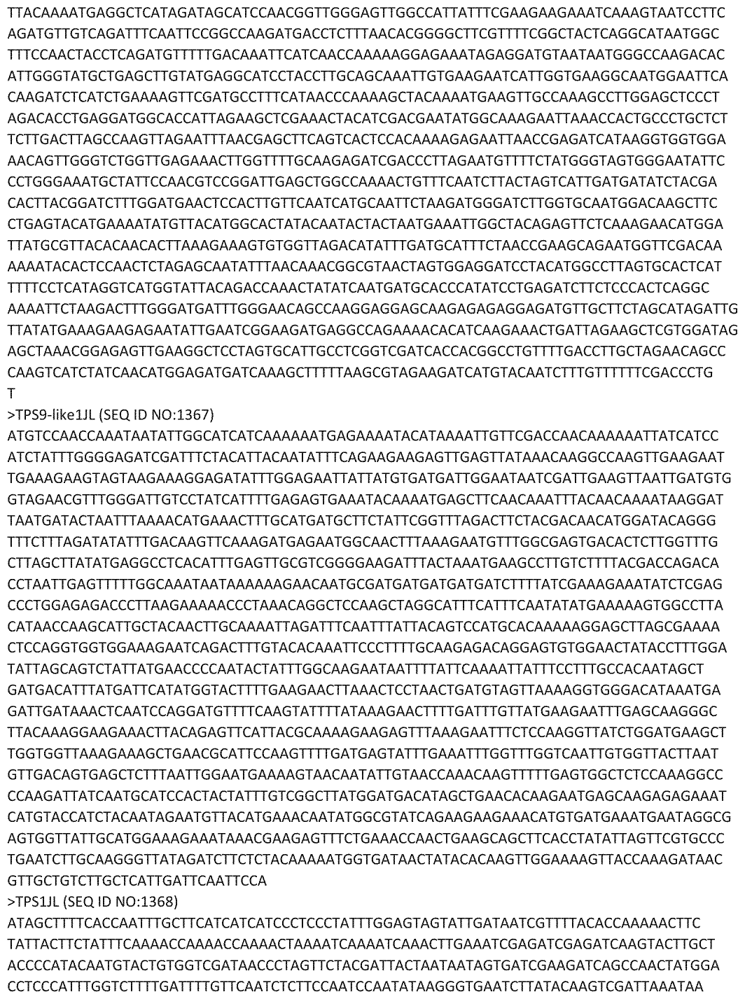

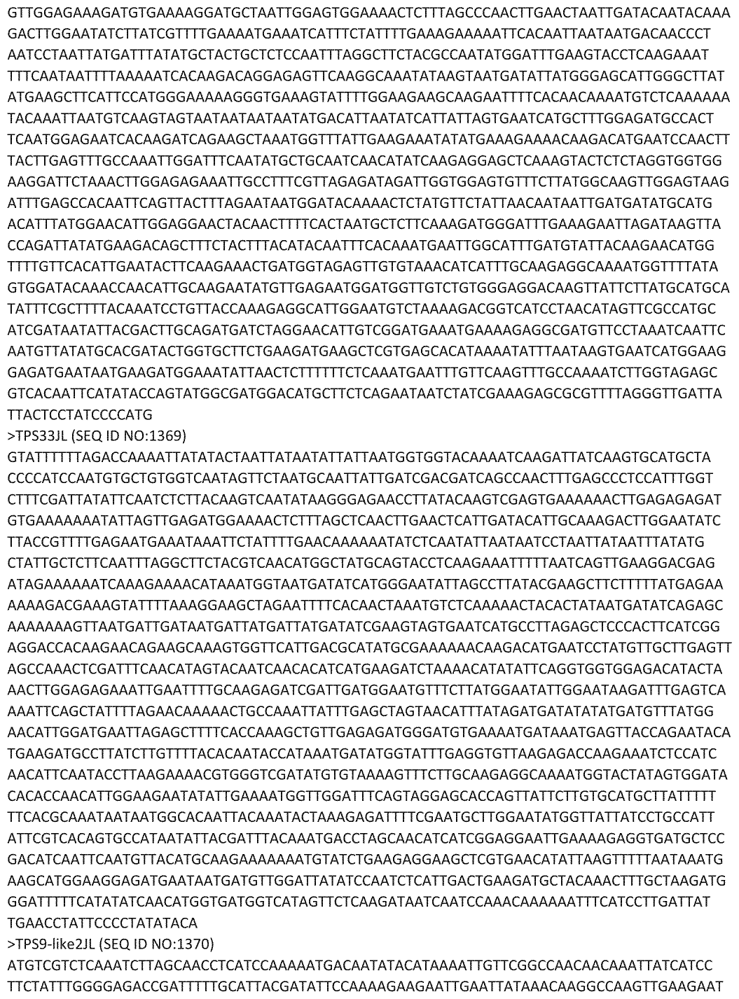

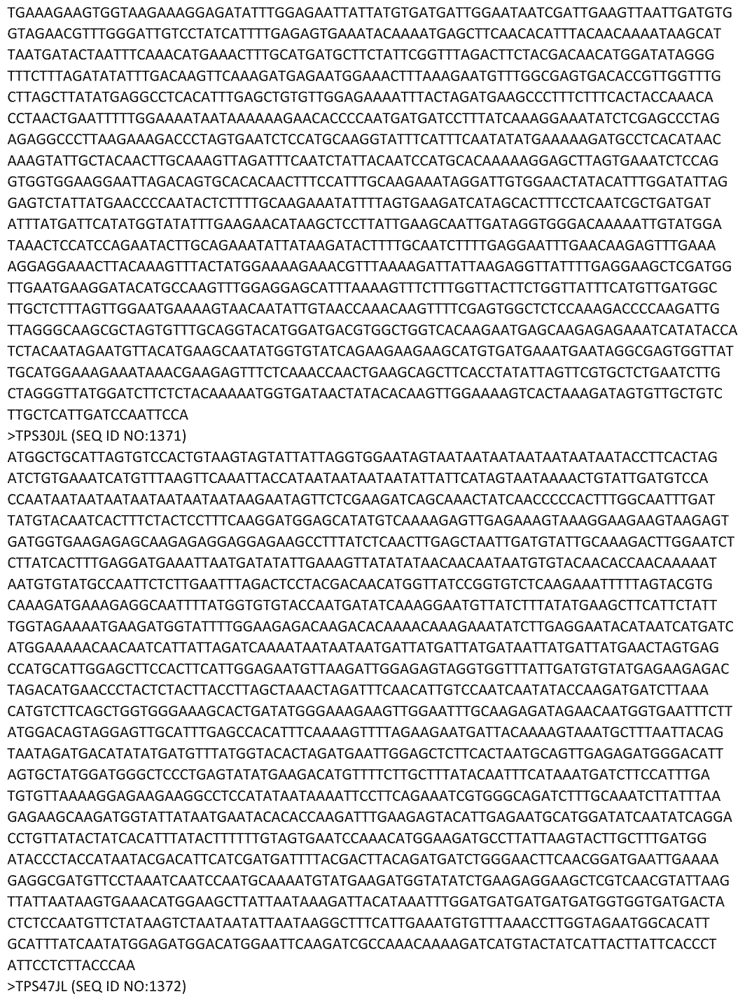

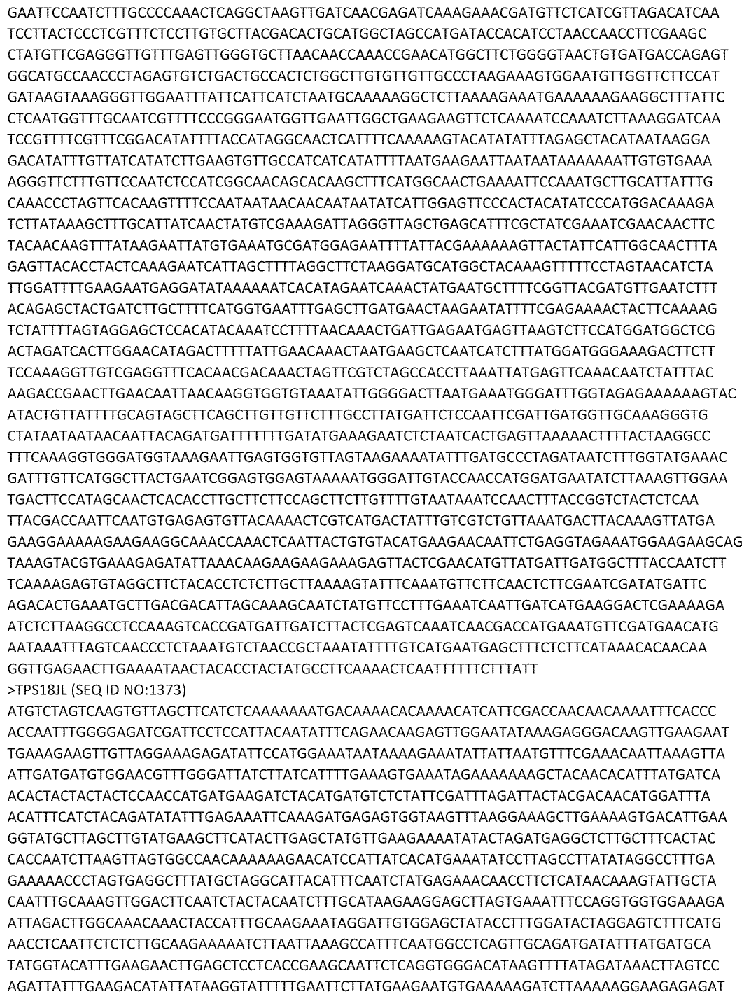

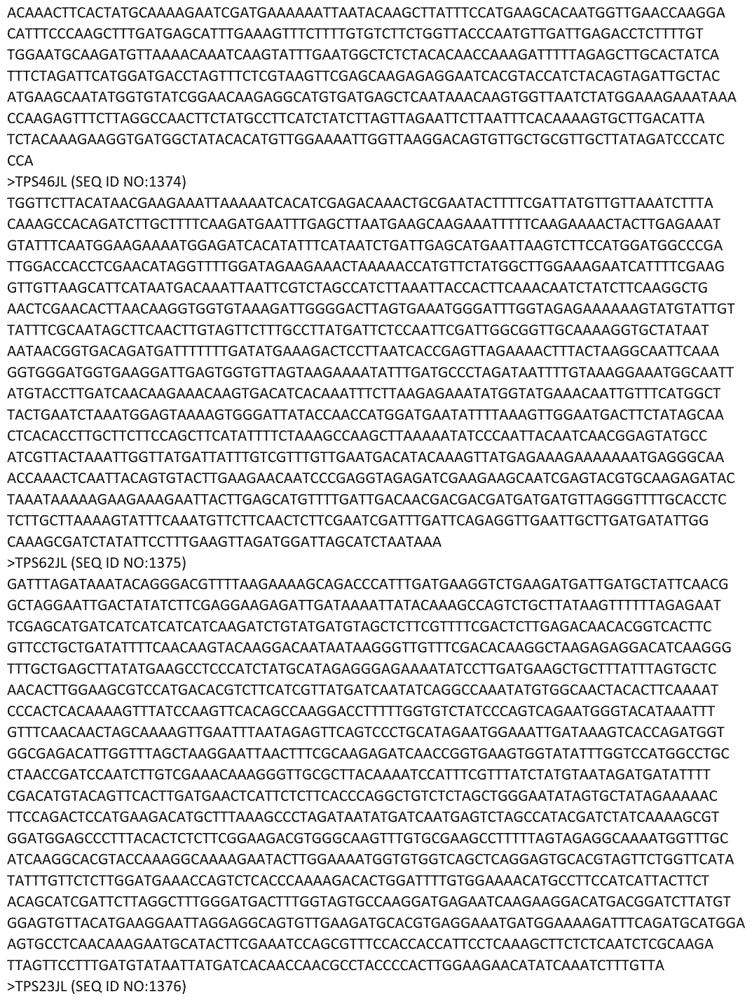

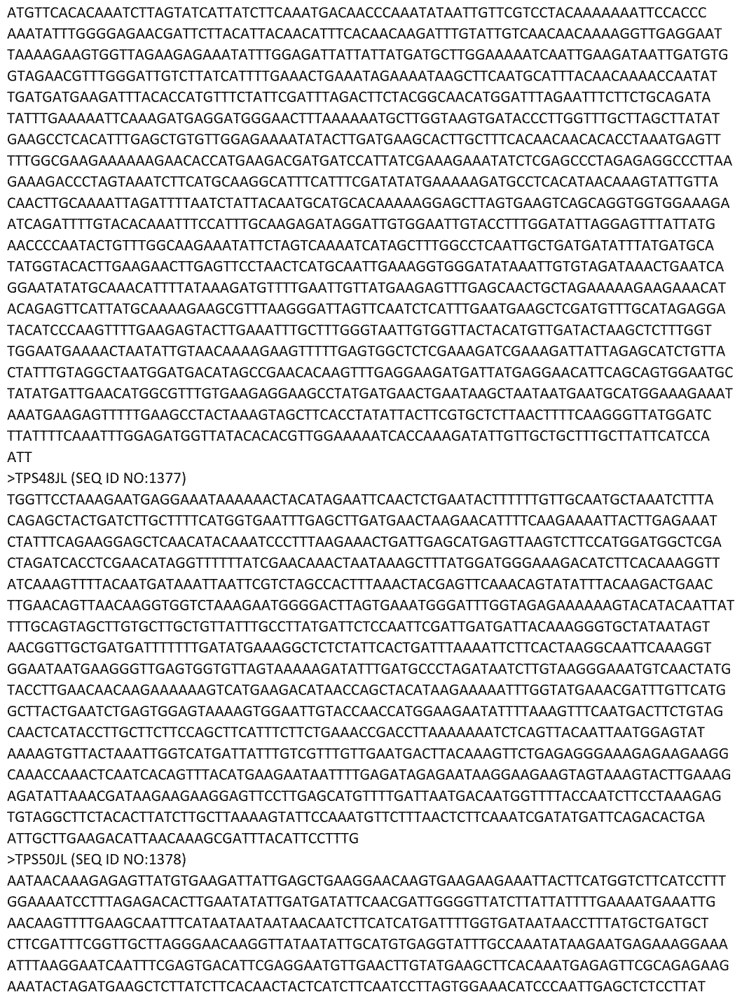

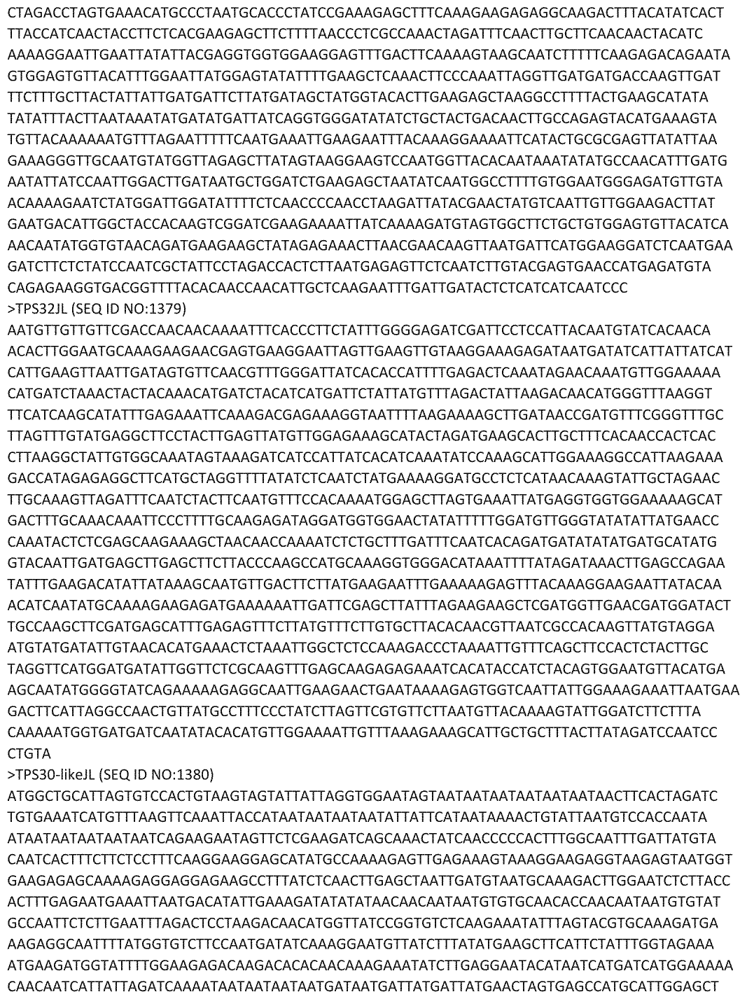

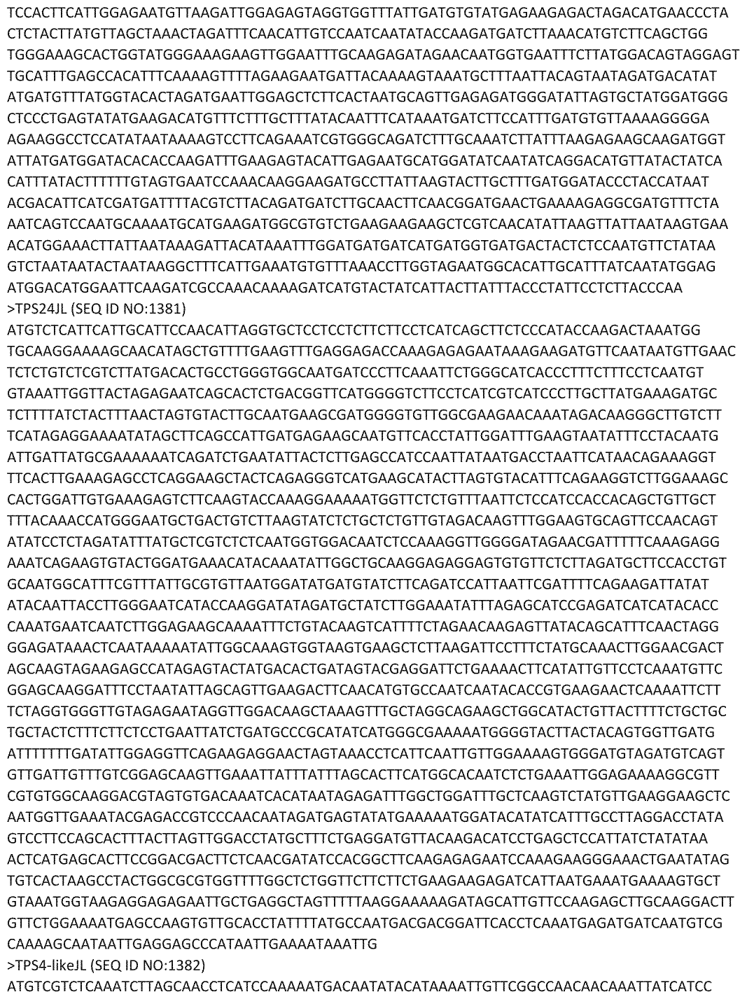

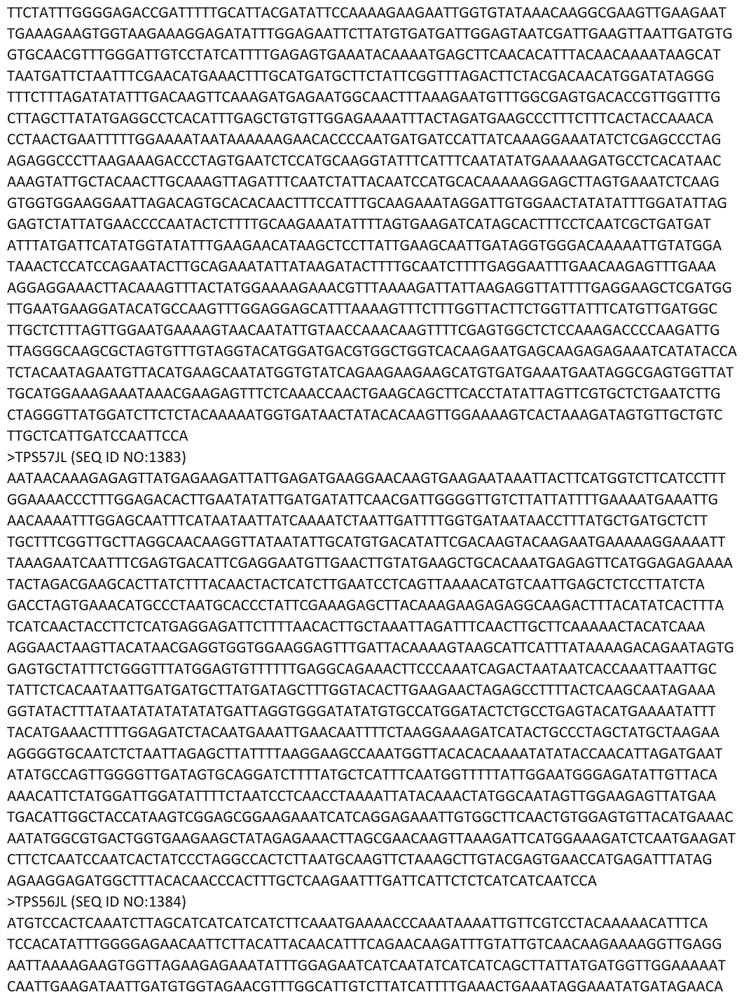

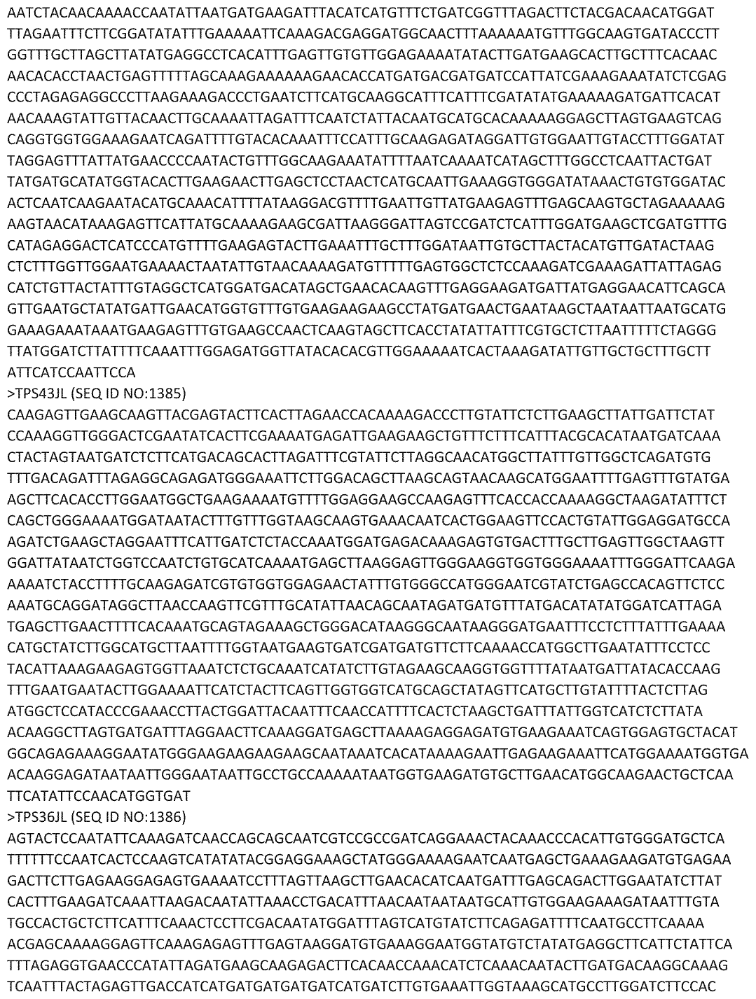

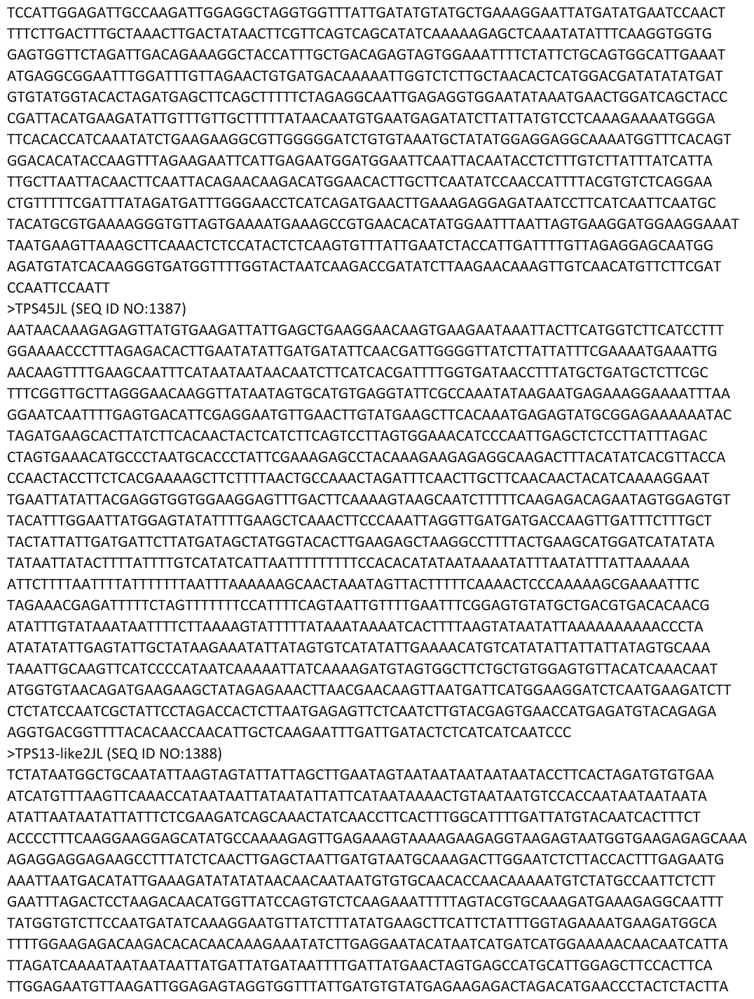

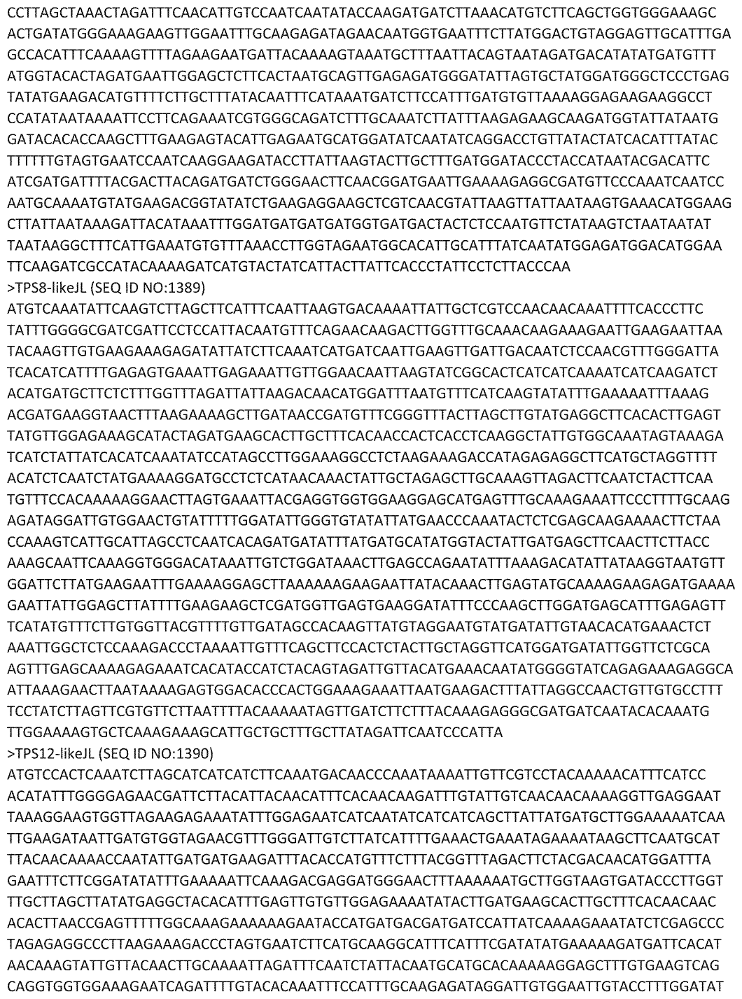

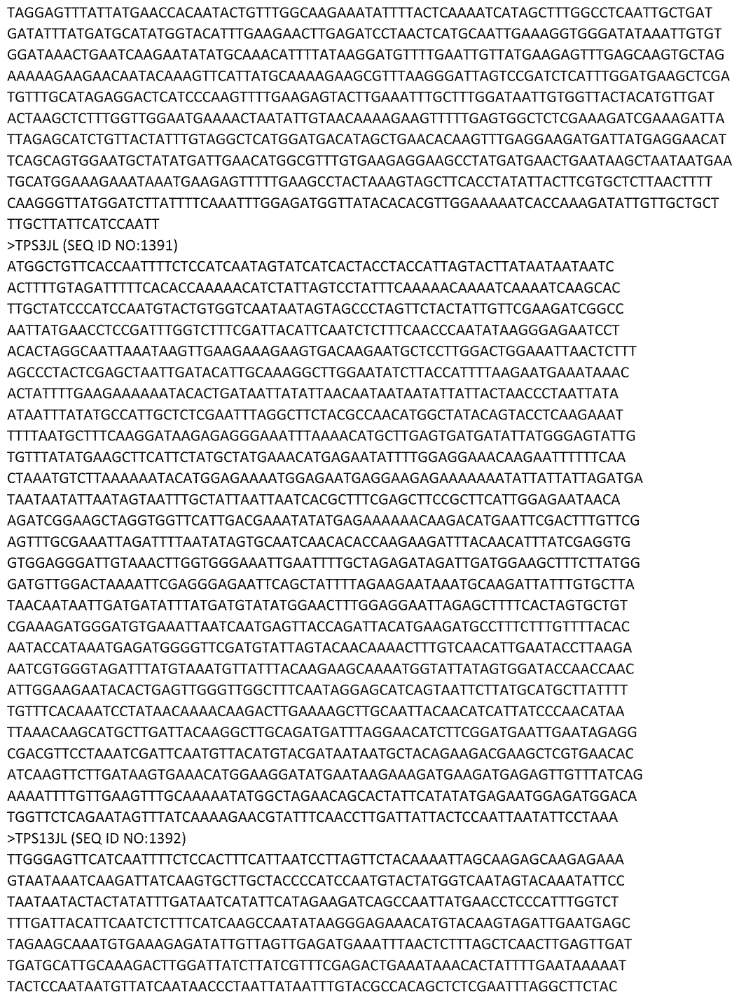

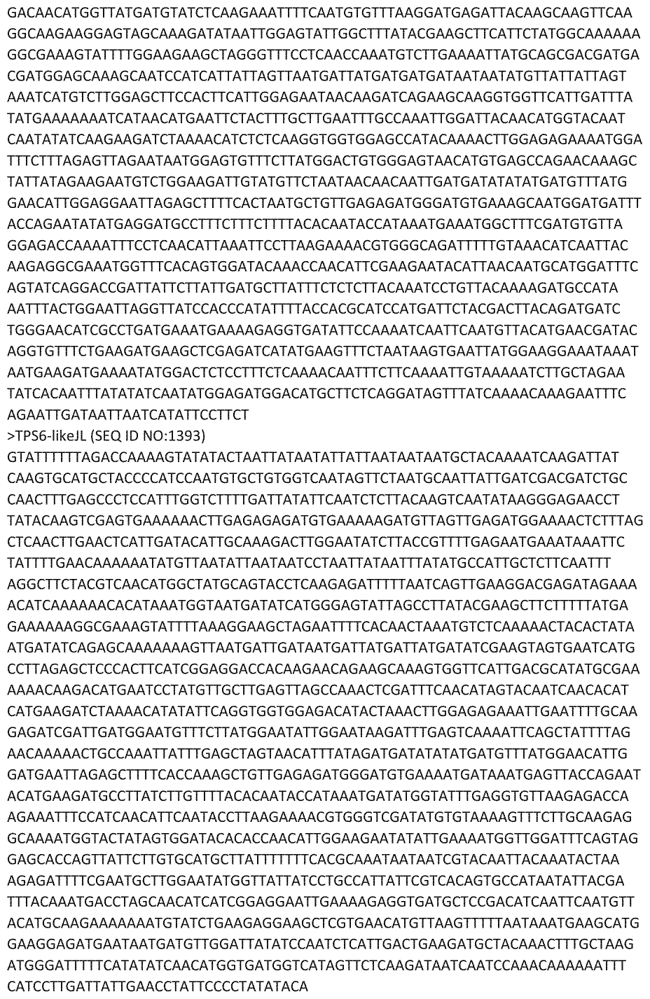

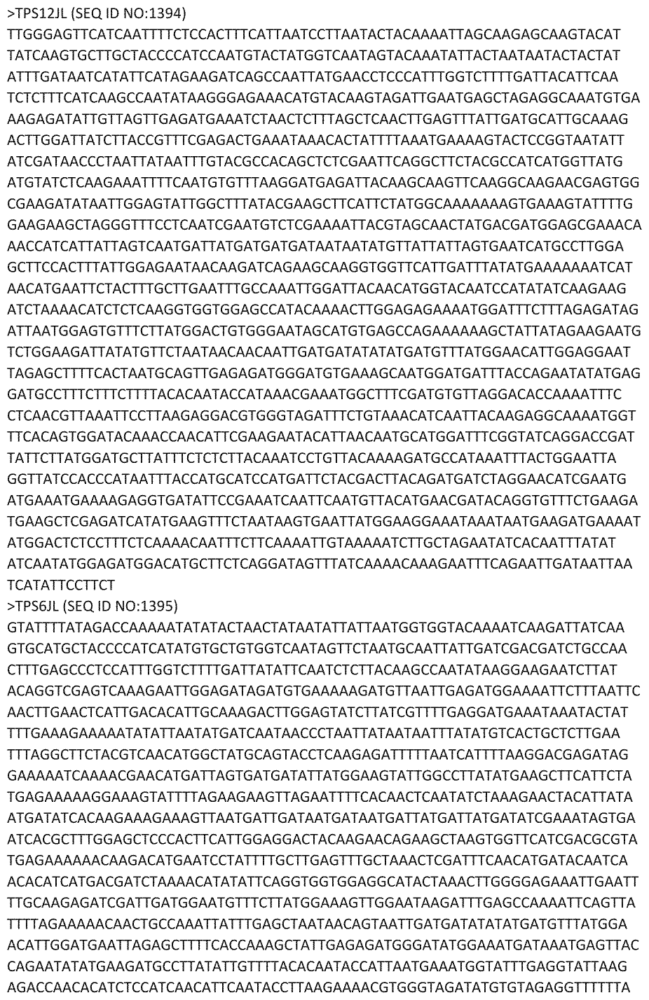

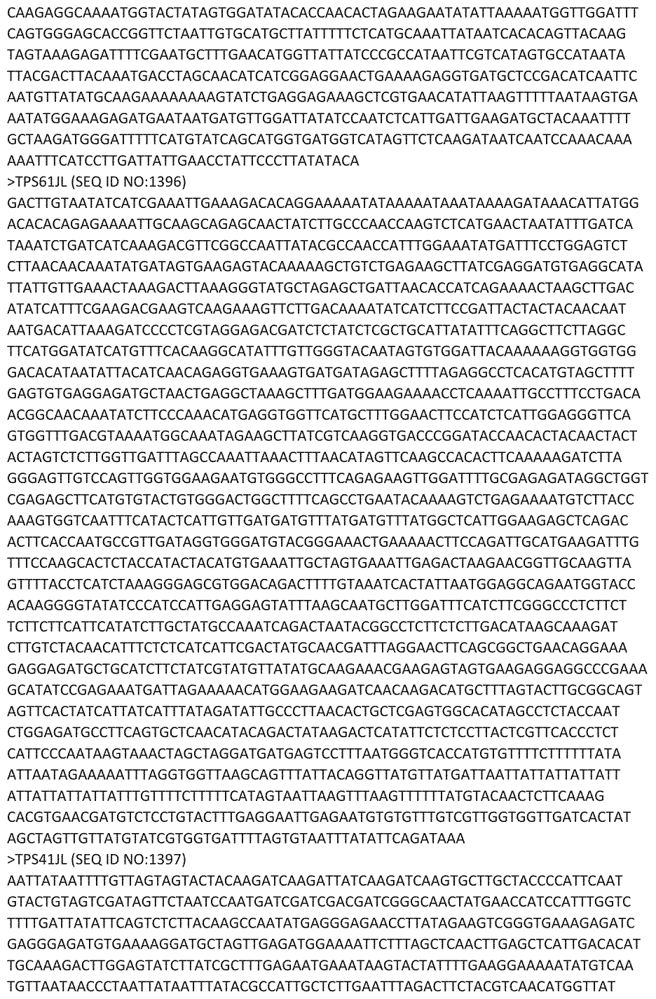

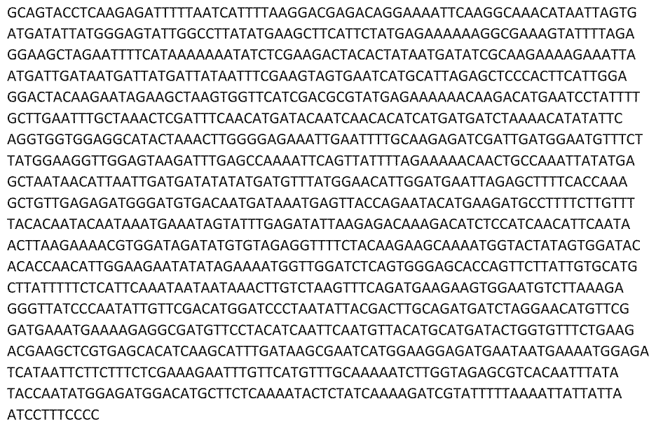

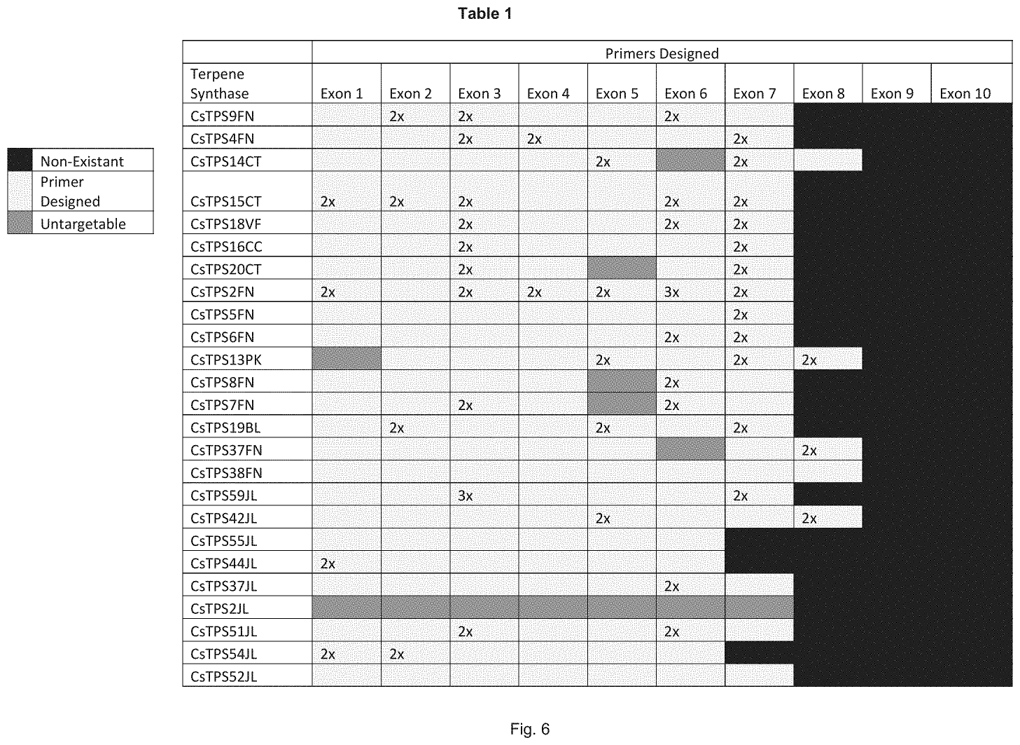

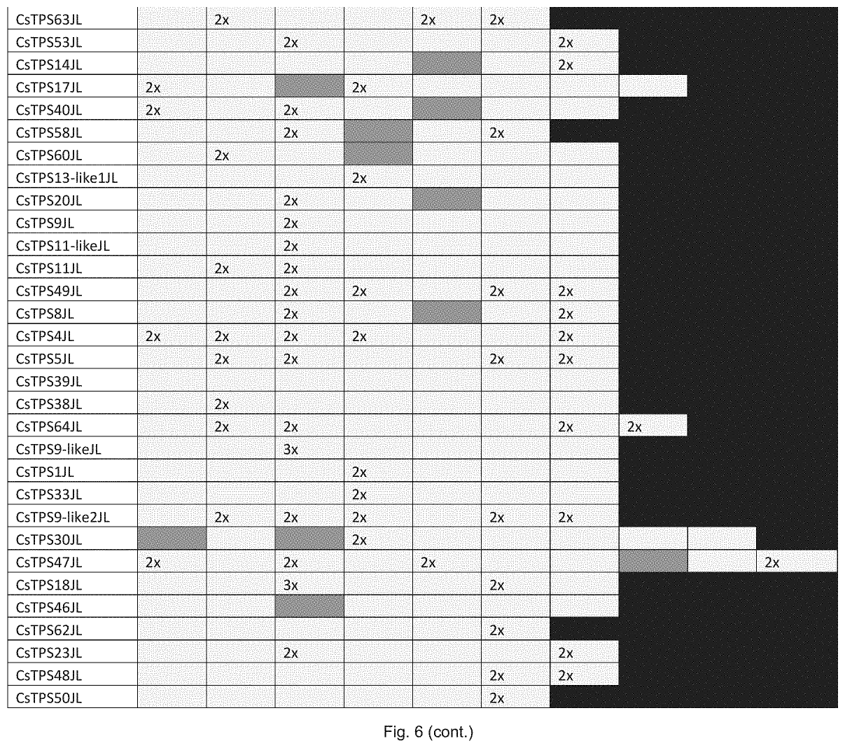

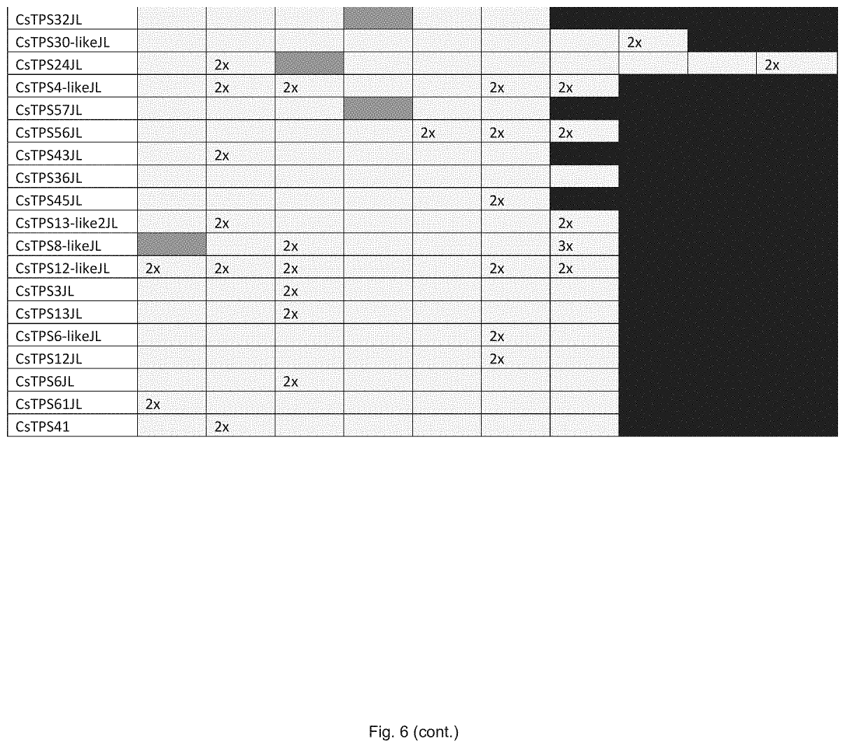

Using this design process, certain exons were identified that could not be targeted due to their homology to other loci in the genome. The Table provided in FIG. 6 (Table 1) is coded for each exon that could be targeted (light grey) and for each exon that could not be targeted (dark grey) for 75 terpene synthase paralogs. The design of 2 primer sets for a single exon is denoted by 2× and the design of 3 primer sets for a single exon is denoted by 3×. The blacked-out exons indicate that the genes do not contain those exons.

The primer sequences that were designed, and their corresponding target terpene synthase paralogs, are set forth in the Table 2 below. These primers specifically targeted the identified coding regions (exons) of the terpene synthase genes but could also be used to examine informative introns through using other combinations of the primers described herein.

TABLE 2

| Gene name | Exon # | Primer Name | Primer Seq (5'->3') | Seq ID# |
|---|---|---|---|---|
| TPS11JL | 1 | TPS11JL-1F | ATATTCAAAGATCAACCAGCAGC | SEQ ID 1 |
| TPS11JL | 1 | TPS11JL-1R | TGTGGGTTTGTAGTTGCCTGAT | SEQ ID 2 |
| TPS11JL | 2a | TPS11JL-2aF | AGCTATGGGAAAAGAATCAATGAGC | SEQ ID 3 |
| TPS11JL | 2a | TPS11JL-2aR | GAGTTTGAAATGAAGAGCAGTGG | SEQ ID 4 |
| TPS11JL | 2b | TPS11JL-2bF | GAGGCTGAAAATCCTTTAGTTAAGC | SEQ ID 5 |
| TPS11JL | 2b | TPS11JL-2bR | ACAGGACTGAATCCATATTGTCTAAGG | SEQ ID 6 |
| TPS11JL | 3a | TPS11JL-3aF | AGATGAAGCAAGAGATTTCACAACC | SEQ ID 7 |
| TPS11JL | 3a | TPS11JL-3aR | AATGGAGTGGAAGATCCAAGGC | SEQ ID 8 |
| TPS11JL | 3b | TPS11JL-3bF | ATGCCTTGGATCTTCCACTCC | SEQ ID 9 |
| TPS11JL | 3b | TPS11JL-3bR | CTTTTTGGTATGCTGACTGAACG | SEQ ID 10 |
| TPS11JL | 4 | TPS11JL-4F | CATTTGCTAGAGACAGAGTAGTGG | SEQ ID 11 |
| TPS11JL | 4 | TPS11JL-4R | GCTGAAGCTCATCTAGTGTACC | SEQ ID 12 |
| TPS11JL | 5 | TPS11JL-5F | TGAACTGGATCAGCTACCCG | SEQ ID 13 |
| TPS11JL | 5 | TPS11JL-5R | GTGTGAATCCCATTTTCTTTGAGG | SEQ ID 14 |
| TPS11JL | 6 | TPS11JL-6F | GTTGGGTGATCTGTGTAAATGC | SEQ ID 15 |
| TPS11JL | 6 | TPS11JL-6R | CTGAGACACGTAAAATGGTTGG | SEQ ID 16 |
| TPS11JL | 7 | TPS11JL-7F | TGCTACATGCGTGAAAAGGG | SEQ ID 17 |
| TPS11JL | 7 | TPS11JL-7R | TGTGGTACATCTCCATTGCTCC | SEQ ID 18 |
| TPS11-like1JL | 1 | TPS11-like1JL-1F | ATGTGCTGTGGTCAATAGTTCT | SEQ ID 19 |
| TPS11-like1JL | 1 | TPS11-like1JL-1R | AAAGACCAAATGGAGGGCTCA | SEQ ID 20 |
| TPS11-like1JL | 2 | TPS11-like1JL-2F | ACAGGTCGAGTCAAAGAATTGG | SEQ ID 21 |
| TPS11-like1JL | 2 | TPS11-like1JL-2R | GCCATGTTGACGTAGAAGCC | SEQ ID 22 |
| TPS11-like1JL | 3a | TPS11-like1JL-3aF | AGGAAGAGATAGGAAAAATCAAAGCG | SEQ ID 23 |
| TPS11-like1JL | 3a | TPS11-like1JL-3aR | GCGTCGATGAACCACTTAGC | SEQ ID 24 |
| TPS11-like1JL | 3b | TPS11-like1JL-3bF | TAAGGAAGAGATAGGAAAAATCAAAGC | SEQ ID 25 |
| TPS11-like1JL | 3b | TPS11-like1JL-3bR | TTGTAGTCCTCCGATGAAGTGG | SEQ ID 26 |
| TPS11-like1JL | 4 | TPS11-like1JL-4F | TGGAGGCATACTAAACTTGGGG | SEQ ID 27 |
| TPS11-like1JL | 4 | TPS11-like1JL-4R | AAGCTCTAATTCATCCAATGTTCC | SEQ ID 28 |
| TPS11-like1JL | 5 | TPS11-like1JL-5F | GATGGGATATGGAAATGATAAATGAGT | SEQ ID 29 |
| TPS11-like1JL | 5 | TPS11-like1JL-5R | TGTTGATGGAGATGTGTTGGTCT | SEQ ID 30 |
| TPS11-like1JL | 6 | TPS11-like1JL-6F | GGATTTCAGTGGGAGCACCG | SEQ ID 31 |
| TPS11-like1JL | 6 | TPS11-like1JL-6R | GCACTATGACGAATTATGGCGG | SEQ ID 32 |
| TPS11-like1JL | 7 | TPS11-like1JL-7F | TGAAAAGAGGTGATGCTCCG | SEQ ID 33 |
| TPS11-like1JL | 7 | TPS11-like1JL-7R | CCATGCTGATACATGAAAAATCCC | SEQ ID 34 |
| TPS12JL | 1 | TPS12JL-1F | ATCCTTAATACTACAAAATTAGCAAGAGC | SEQ ID 35 |
| TPS12JL | 1 | TPS12JL-1R | AATGTAATCAAAAGACCAAATGGGAGG | SEQ ID 36 |
| TPS12JL | 2 | TPS12JL-2F | AGTAGATTGAATGAGCTAGAGGC | SEQ ID 37 |
| TPS12JL | 2 | TPS12JL-2R | AATTCGAGAGCTGTGGCG | SEQ ID 38 |
| TPS12JL | 3 | TPS12JL-3F | AAGCAAGTTCAAGGCAAGAACG | SEQ ID 39 |
| TPS12JL | 3 | TPS12JL-3R | GATGGTTTGTTTCGCTCCATCG | SEQ ID 40 |
| TPS12JL | 4 | TPS12JL-4F | GGAGTGTTTCTTATGGACTGTGG | SEQ ID 41 |
| TPS12JL | 4 | TPS12JL-4R | GAAAAGCTCTAATTCCTCCAATGTTCC | SEQ ID 42 |
| TPS12JL | 5 | TPS12JL-5F | TGTGAAAGCAATGGATGATTTACC | SEQ ID 43 |
| TPS12JL | 5 | TPS12JL-5R | AATTTTGGTGTCCTAACACATCG | SEQ ID 44 |
| TPS12JL | 6a | TPS12JL-6aF | TCTGTAAACATCAATTACAAGAGGC | SEQ ID 45 |
| TPS12JL | 6a | TPS12JL-6aR | TCGGTCCTGATACCGAAATCC | SEQ ID 46 |
| TPS12JL | 6b | TPS12JL-6bF | ATGGATTTCGGTATCAGGACCG | SEQ ID 47 |
| TPS12JL | 6b | TPS12JL-6bR | CATCTGTAAGTCGTAGAATCATGG | SEQ ID 48 |
| TPS12JL | 7 | TPS12JL-7F | TAATGAAGATGAAAATATGGACTCTCC | SEQ ID 49 |
| TPS12JL | 7 | TPS12JL-7R | GATAAACTATCCTGAGAAGCATGTCC | SEQ ID 50 |
| TPS12-likeJL | 1a | TPS12-likeJL-1aF | TGTCCACTCAAATCTTAGCATCA | SEQ ID 51 |
| TPS12-likeJL | 1a | TPS12-likeJL-1aR | TGAAATGTTTTTGTAGGACGAACA | SEQ ID 52 |
| TPS12-likeJL | 1b | TPS12-likeJL-1bF | CAACCCAAATAAAATTGTTCGTCC | SEQ ID 53 |
| TPS12-likeJL | 1b | TPS12-likeJL-1bR | TTGTGAAATGTTGTAATGTAAGAATCG | SEQ ID 54 |
| TPS12-likeJL | 2a | TPS12-likeJL-2aF | TGTCAACAACAAAAGGTTGAGG | SEQ ID 55 |
| TPS12-likeJL | 2a | TPS12-likeJL-2aR | AGTCTAAACCGTAAAGAAACATGG | SEQ ID 56 |
| TPS12-likeJL | 2b | TPS12-likeJL-2bF | AAAGGTTGAGGAATTAAAGGAAGTGG | SEQ ID 57 |
| TPS12-likeJL | 2b | TPS12-likeJL-2bR | TCAAAATGATAAGACAATCCCAAACG | SEQ ID 58 |
| TPS12-likeJL | 3a | TPS12-likeJL-3aF | TGAAAAATTCAAAGACGAGGATGGG | SEQ ID 59 |
| TPS12-likeJL | 3a | TPS12-likeJL-3aR | AAGGGCCTCTCTAGGGCTCG | SEQ ID 60 |
| TPS12-likeJL | 3b | TPS12-likeJL-3bF | ACACTTAACCGAGTTTTTGGC | SEQ ID 61 |
| TPS12-likeJL | 3b | TPS12-likeJL-3bR | TGCTGACTTCACAAAGCTCC | SEQ ID 62 |
| TPS12-likeJL | 4 | TPS12-likeJL-4F | TGCAAGAGATAGGATTGTGGAAT | SEQ ID 63 |
| TPS12-likeJL | 4 | TPS12-likeJL-4R | GCATGAGTTAGGATCTCAAGTTCTTC | SEQ ID 64 |
| TPS12-likeJL | 5 | TPS12-likeJL-5F | TGGGATATAAATTGTGTGGATAAACTG | SEQ ID 65 |
| TPS12-likeJL | 5 | TPS12-likeJL-5R | GCACTTGCTCAAACTCTTCATAACAA | SEQ ID 66 |
| TPS12-likeJL | 6a | TPS12-likeJL-6aF | TGTTTGCATAGAGGACTCATCCC | SEQ ID 67 |
| TPS12-likeJL | 6a | TPS12-likeJL-6aR | AGTTTTCATTCCAACCAAAGAGC | SEQ ID 68 |
| TPS12-likeJL | 6b | TPS12-likeJL-6bF | TTTGGATGAAGCTCGATGTTTGC | SEQ ID 69 |
| TPS12-likeJL | 6b | TPS12-likeJL-6bR | AGTAACAGATGCTCTAATAATCTTTCG | SEQ ID 70 |
| TPS12-likeJL | 7a | TPS12-likeJL-7aF | ATGAGGAACATTCAGCAGTGG | SEQ ID 71 |
| TPS12-likeJL | 7a | TPS12-likeJL-7aR | AGCACGAAGTAATATAGGTGAAGC | SEQ ID 72 |
| TPS12-likeJL | 7b | TPS12-likeJL-7bF | ATGGCGTTTGTGAAGAGGAAGC | SEQ ID 73 |
| TPS12-likeJL | 7b | TPS12-likeJL-7bR | AATTGGATGAATAAGCAAAGCAGC | SEQ ID 74 |
| TPS13JL | 1 | TPS13JL-1F | TCCTTAGTTCTACAAAATTAGCAAGAGC | SEQ ID 75 |
| TPS13JL | 1 | TPS13JL-1R | GTAATCAAAAGACCAAATGGGAGG | SEQ ID 76 |
| TPS13JL | 2 | TPS13JL-2F | ACAAGTAGATTGAATGAGCTAGAAGC | SEQ ID 77 |
| TP513JL | 2 | TPS13JL-2R | TCATAACCATGTTGTCGTAGAAGC | SEQ ID 78 |
| TPS13JL | 3a | TPS13JL-3aF | GTTCAAGGCAAGAAGGAGTAGC | SEQ ID 79 |
| TPS13JL | 3a | TPS13JL-3aR | TTTGCTCCATCGTCATCGTCG | SEQ ID 80 |
| TPS13JL | 3b | TPS13JL-3bF | ATTATGCAGCGACGATGACG | SEQ ID 81 |
| TPS13JL | 3b | TPS13JL-3bR | CCTTGCTTCTGATCTTGTTATTCTCC | SEQ ID 82 |
| TPS13JL | 4 | TPS13JL-4F | TGGAGTGTTTCTTATGGACTGTGG | SEQ ID 83 |
| TPS13JL | 4 | TPS13JL-4R | CTCTCAACAGCATTAGTGAAAAGC | SEQ ID 84 |
| TPS13JL | 5 | TPS13JL-5F | TGTGAAAGCAATGGATGATTTACC | SEQ ID 85 |
| TPS13JL | 5 | TPS13JL-5R | AATTTTGGTCTCCTAACACATCG | SEQ ID 86 |
| TPS13JL | 6 | TPS13JL-6F | GGCGAAATGGTTTCACAGTGG | SEQ ID 87 |
| TPS13JL | 6 | TPS13JL-6R | CGTAGAATCATGGATGCGTGG | SEQ ID 88 |
| TPS13JL | 7 | TPS13JL-7F | AAATAATGAAGATGAAAATATGGACTCTCC | SEQ ID 89 |
| TPS13JL | 7 | TPS13JL-7R | GATAAACTATCCTGAGAAGCATGTCC | SEQ ID 90 |
| TPS13-like1JL | 1 | TPS13-like1JL-1F | AAACTATCAACCTTCACTTTGGC | SEQ ID 91 |
| TPS13-like1JL | 1 | TPS13-like1JL-1R | TTCCTCTTTTGCTCTCTTCACC | SEQ ID 92 |
| TPS13-like1JL | 2 | TPS13-like1JL-2F | TGCAAAGACTTGGAATCTCTTATCAC | SEQ ID 93 |
| TPS13-like1JL | 2 | TPS13-like1JL-2R | GAGACACCGGATAACCATGTTG | SEQ ID 94 |
| TPS13-like1JL | 3 | TPS13-like1JL-3F | GAGGCAATTTTATGGTGTGTACC | SEQ ID 95 |
| TPS13-like1JL | 3 | TPS13-like1JL-3R | TCTTTGTTGTTTCTCTTGTCTCTTCC | SEQ ID 96 |
| TPS13-like1JL | 4a | TPS13-like1JL-4aF | TGGGAAAGCACTGGTATGGG | SEQ ID 97 |
| TPS13-like1JL | 4a | TPS13-like1JL-4aR | AGCTCCAATTCATCTAGTGTACC | SEQ ID 98 |
| TPS13-like1JL | 4b | TPS13-like1JL-4bF | GTGGGAAAGCACTGGTATGGG | SEQ ID 99 |
| TPS13-like1JL | 4b | TPS13-like1JL-4bR | AATTTTTGTAATGTGGCTCAAATGC | SEQ ID 100 |
| TPS13-like1JL | 5 | TPS13-like1JL-5F | TGGGATATTAGTGCTATGGATGGG | SEQ ID 101 |
| TPS13-like1JL | 5 | TPS13-like1JL-5R | TTCTGAAGGAATTTTATTATATGGAGGC | SEQ ID 102 |
| TPS13-like1JL | 6 | TPS13-like1JL-6F | TAGTGAATCCAAACAAGGAAGATGC | SEQ ID 103 |
| TPS13-like1JL | 6 | TPS13-like1JL-6R | TCATCTGTAAGACGTAAAATCATCG | SEQ ID 104 |
| TPS13-like1JL | 7 | TPS13-like1JL-7F | GATGGCGTGTTTGAAGAAGAAGC | SEQ ID 105 |
| TPS13-like1JL | 7 | TPS13-like1JL-7R | TGATAGTACATGATCTTTTGTTTGGC | SEQ ID 106 |
| TPS13-like2JL | 1 | TPS13-like2JL-1F | TTCTCGAAGATCAGCAAACTATCAAC | SEQ ID 107 |
| TPS13-like2JL | 1 | TPS13-like2JL-1R | TTGAAAGGGGTAGAAAGTGATTGTA | SEQ ID 108 |
| TPS13-like2JL | 2a | TPS13-like2JL-2aF | AGGTAAGAGTAATGGTGAAGAGAGC | SEQ ID 109 |
| TPS13-like2JL | 2a | TPS13-like2JL-2aR | CATTCTCAAAGTGGTAAGAGATTCC | SEQ ID 110 |
| TPS13-like2JL | 2b | TPS13-like2JL-2bF | GAGCAAAAGAGGAGGAGAAGCC | SEQ ID 111 |
| TPS13-like2JL | 2b | TPS13-like2JL-2bR | GGCATAGACATTTTTGTTGGTGTTGC | SEQ ID 112 |
| TPS13-like2JL | 3 | TPS13-like2JL-3F | GAGGCAATTTTATGGTGTCTTCC | SEQ ID 113 |
| TPS13-like2JL | 3 | TPS13-like2JL-3R | TGTTGTGTGTCTTGTCTCTTCC | SEQ ID 114 |
| TPS13-like2JL | 4 | TPS13-like2JL-4F | TGGGAAAGCACTGATATGGG | SEQ ID 115 |
| TPS13-like2JL | 4 | TPS13-like2JL-4R | AGCTCCAATTCATCTAGTGTACC | SEQ ID 116 |
| TPS13-like2JL | 5 | TPS13-like2JL-5F | TGGGATATTAGTGCTATGGATGGG | SEQ ID 117 |
| TPS13-like2JL | 5 | TPS13-like2JL-5R | TTCTGAAGGAATTTTATTATATGGAGGC | SEQ ID 118 |
| TPS13-like2JL | 6 | TPS13-like2JL-6F | AGCTTTGAAGAGTACATTGAGAATGC | SEQ ID 119 |
| TPS13-like2JL | 6 | TPS13-like2JL-6R | ATCATCTGTAAGTCGTAAAATCATCG | SEQ ID 120 |
| TPS13-like2JL | 7a | TPS13-like2JL-7aF | GGCGATGTTCCCAAATCAATCC | SEQ ID 121 |
| TPS13-like2JL | 7a | TPS13-like2JL-7aR | AACATTGGAGAGTAGTCATCACC | SEQ ID 122 |
| TPS13-like2JL | 7b | TPS13-like2JL-7bF | GATGATGATGGTGATGACTACTCTCC | SEQ ID 123 |
| TPS13-like2JL | 7b | TPS13-like2JL-7bR | GATAGTACATGATCTTTTGTATGGCG | SEQ ID 124 |
| TPS13PK | 2 | TPS13PK-2F | GATCAGCAAACTATCAACCCCC | SEQ ID 125 |
| TPS13PK | 2 | TPS13PK-2R | TCACCATCACTCTTACTTCTTCC | SEQ ID 126 |
| TPS13PK | 3 | TPS13PK-3F | ACTTGGAATCTCTTATCACTTTGAGG | SEQ ID 127 |
| TPS13PK | 3 | TPS13PK-3R | AGAGAATTGGCATACACATTATTATTGG | SEQ ID 128 |
| TPS13PK | 4 | TPS13PK-4F | GAGGCAATTTTATGGTGTGTACC | SEQ ID 129 |
| TPS13PK | 4 | TPS13PK-4R | TTTCTCTTGTCTCTTCCAAAATACC | SEQ ID 130 |
| TPS13PK | 5a | TPS13PK-5aF | TGGTGGGAAAGCACTGATATGG | SEQ ID 131 |
| TPS13PK | 5a | TPS13PK-5aR | AATGTGGCTCAAATGCAACTCC | SEQ ID 132 |
| TPS13PK | 5b | TPS13PK-5bF | TGGGAAAGCACTGATATGGG | SEQ ID 133 |
| TPS13PK | 5b | TPS13PK-5bR | AGCTCCAATTCATCTAGTGTACC | SEQ ID 134 |
| TPS13PK | 6 | TPS13PK-6F | TGGGATATTAGTGCTATGGATGGG | SEQ ID 135 |
| TPS13PK | 6 | TPS13PK-6R | TTCTGAAGGAATTTTATTATATGGAGGC | SEQ ID 136 |
| TPS13PK | 7a | TPS13PK-7aF | GCTTTGAAGAGTACATTGAGAATGC | SEQ ID 137 |
| TPS13PK | 7a | TPS13PK-7aR | CGATGAATGTCGTATTATGGTAGGG | SEQ ID 138 |
| TPS13PK | 7b | TPS13PK-7bF | GTGAATCCAAACAAGGAAAATGCC | SEQ ID 139 |
| TPS13PK | 7b | TPS13PK-7bR | TGAAGTTCCCATATCATCTTTAAGTCG | SEQ ID 140 |
| TPS13PK | 8a | TPS13PK-8aF | TGAATTGAAAAGAGGCGATGTTCC | SEQ ID 141 |
| TPS13PK | 8a | TPS13PK-8aR | ACATTGGAGAGTAGTCATCACC | SEQ ID 142 |
| TPS13PK | 8b | TPS13PK-8bF | TGGTATATCTGAAGAGGAAGCTCG | SEQ ID 143 |
| TPS13PK | 8b | TPS13PK-8bR | AGAACATTGGAGAGTAGTCATCACC | SEQ ID 144 |
| TPS14CT | 1 | TPS14CT-1F | GCATAGCTTTTCACCAATTTGC | SEQ ID 145 |
| TPS14CT | 1 | TPS14CT-1R | GGGAGGGATGATGATGAAGCA | SEQ ID 146 |
| TPS14CT | 2 | TPS14CT-2F | CAATGTACTGTGGTCGATAACCC | SEQ ID 147 |
| TPS14CT | 2 | TPS14CT-2R | GAACAAAATCAAAAGACCAAATGGG | SEQ ID 148 |
| TPS14CT | 3 | TPS14CT-3F | TGGAGAAAGATGTGAAAAGGATGC | SEQ ID 149 |
| TPS14CT | 3 | TPS14CT-3R | ATTGGCGTAGAAGCCTAAATTGG | SEQ ID 150 |
| TPS14CT | 4 | TPS14CT-4F | TCACAAGACAGGAGAGTTCAAGG | SEQ ID 151 |
| TPS14CT | 4 | TPS14CT-4R | TGAAGTGGCATCTCCAAAGC | SEQ ID 152 |
| TPS14CT | 5a | TPS14CT-5aF | TGGTGGAAGGATTCTAAACTTGG | SEQ ID 153 |
| TPS14CT | 5a | TPS14CT-5aR | CTCCAATGTTCCATAAATGTCATGC | SEQ ID 154 |
| TPS14CT | 5b | TPS14CT-5bF | GCAAGTTGGAGTAAGATTTGAGC | SEQ ID 155 |
| TPS14CT | 5b | TPS14CT-5bR | ATTAGTGAAAAGTTGTAGTTCCTCC | SEQ ID 156 |
| TPS14CT | 7a | TPS14CT-7aF | AAGAGGCAAAATGGTTTTATAGTGG | SEQ ID 157 |
| TPS14CT | 7a | TPS14CT-7aR | TTGGTAACAGGATTTGTAAAAGCG | SEQ ID 158 |
| TPS14CT | 7b | TPS14CT-7bF | GGATGGTTGTCTGTGGGAGG | SEQ ID 159 |
| TPS14CT | 7b | TPS14CT-7bR | TGCATGGCGAACTATGTTAGG | SEQ ID 160 |
| TPS14CT | 8 | TPS14CT-8F | TGCAAAAATCTTGGTAGAGCG | SEQ ID 161 |
| TPS14CT | 8 | TPS14CT-8R | TGGGGATAGGAGTAATAATCAACCC | SEQ ID 162 |
| TPS14JL | 1 | TPS14JL-1F | ATATTCAAGTCTTAGCTTCATCTCAATTA | SEQ ID 163 |
| TPS14JL | 1 | TPS14JL-1R | ATCGCCCCAAATAGAAGGGTG | SEQ ID 164 |
| TPS14JL | 2 | TPS14JL-2F | TTTGAGAGTGAAATTGAGAAATTGTTGG | SEQ ID 165 |
| TPS14JL | 2 | TPS14JL-2R | TAAATCCATGTTGTCTTAATAATCTAAACC | SEQ ID 166 |
| TPS14JL | 3 | TPS14JL-3F | GGTTTGCTTAGCTTGTATGAGGC | SEQ ID 167 |
| TPS14JL | 3 | TPS14JL-3R | GCCTCTCTATGGTCTTTCTTAGAGG | SEQ ID 168 |
| TPS14JL | 4 | TPS14JL-4F | TGGTGGAAAGAGCATGAGTTTGC | SEQ ID 169 |
| TPS14JL | 4 | TPS14JL-4R | ATTGAGGCTAATGCAATGACTTTGG | SEQ ID 170 |
| TPS14JL | 6 | TPS14JL-6F | AGCTCGATGGTTGAGTGAAGG | SEQ ID 171 |
| TPS14JL | 6 | TPS14JL-6R | CTAGCAAGGAGAGTGGAAGC | SEQ ID 172 |
| TPS14JL | 7a | TPS14JL-7aF | TGAGCAAAAGAGAAATCACATACC | SEQ ID 173 |
| TPS14JL | 7a | TPS14JL-7aR | TTTCTTTCCAGTGGGTGTCC | SEQ ID 174 |
| TPS14JL | 7b | TPS14JL-7bF | TGAAACAATATGGGGTATCAGAGG | SEQ ID 175 |
| TPS14JL | 7b | TPS14JL-7bR | AATGCTTTCTTTGAGCACTTTTCC | SEQ ID 176 |
| TPS15CT | 1a | TPS15CT-1aF | GCATTGTATGGCTGTTCACC | SEQ ID 177 |
| TPS15CT | 1a | TPS15CT-1aR | TCGAAAGACCAAATCGGAGG | SEQ ID 178 |
| TPS15CT | 1b | TPS15CT-lbF | TCACACCAAAAACATCTATTAGTCC | SEQ ID 179 |
| TPS15CT | 1b | TPS15CT-lbR | CAAATCGGAGGTTCATAATTGGC | SEQ ID 180 |
| TPS15CT | 2a | TPS15CT-2aF | AAGAAAGAAGTGACAAGAATGCTCC | SEQ ID 181 |
| TPS15CT | 2a | TPS15CT-2aR | ACTGTATAGCCATGTTGGCG | SEQ ID 182 |
| TPS15CT | 2b | TPS15CT-2bF | GAAATTAACTCTTTAGCCCTACTCG | SEQ ID 183 |
| TPS15CT | 2b | TPS15CT-2bR | AGCCTAAATTCGAGAGCAATGG | SEQ ID 184 |
| TPS15CT | 3a | TPS15CT-3aF | TGCTTTCAAGGATAAGAGAGGG | SEQ ID 185 |
| TPS15CT | 3a | TPS15CT-3aR | TCTTCCTCATTCTCCATTTTCTCC | SEQ ID 186 |
| TPS15CT | 3b | TPS15CT-3bF | CATGGAGAAAATGGAGAATGAGG | SEQ ID 187 |
| TPS15CT | 3b | TPS15CT-3bR | TCGCAAACTCGAACAAAGTCG | SEQ ID 188 |
| TPS15CT | 4 | TPS15CT-4F | TTGCTAGAGATAGATTGATGGAAGC | SEQ ID 189 |
| TPS15CT | 4 | TPS15CT-4R | AAAGCTCTAATTCCTCCAAAGTTCC | SEQ ID 190 |
| TPS15CT | 5 | TPS15CT-5F | ACCAGATTACATGAAGATGCCTT | SEQ ID 191 |
| TPS15CT | 5 | TPS15CT-5R | CTAATACATCGAACCCCATCTCA | SEQ ID 192 |
| TPS15CT | 6a | TPS15CT-6aF | GGTATTATAGTGGATACCAACCAAC | SEQ ID 193 |
| TPS15CT | 6a | TPS15CT-6aR | AAGCCAACCCAACTCAGTGT | SEQ ID 194 |
| TPS15CT | 6b | TPS15CT-6bF | ACACTGAGTTGGGTTGGCTTT | SEQ ID 195 |
| TPS15CT | 6b | TPS15CT-6bR | GTTCCTAAATCATCTGCAAGCCT | SEQ ID 196 |
| TPS15CT | 7 | TPS15CT-7F | TTGAATAGAGGCGACGTTCC | SEQ ID 197 |
| TPS15CT | 7 | TPS15CT-7R | TGCTGTTCTAGCCATATTTTTGC | SEQ ID 198 |
| TPS16CC | 1 | TPS16CC-1F | TCTAGTCAAGTGTTAGCTTCATCTC | SEQ ID 199 |
| TPS16CC | 1 | TPS16CC-1R | TTTGTTGTTGGTCGAATGATGT | SEQ ID 200 |
| TPS16CC | 2 | TPS16CC-2F | AGGGACAAGTTGAAGAATTGAAAGA | SEQ ID 201 |
| TPS16CC | 2 | TPS16CC-2R | CATGGTTGGAGTAGTAGTAGTGT | SEQ ID 202 |
| TPS16CC | 3a | TPS16CC-3aF | GAGAGTGGTAAGTTTAAGGAAAGC | SEQ ID 203 |
| TPS16CC | 3a | TPS16CC-3aR | CCTAGCATAAAGCCTCACTAGG | SEQ ID 204 |
| TPS16CC | 3b | TPS16CC-3bF | GGCTCTTGCTTTCACTACCACC | SEQ ID 205 |
| TPS16CC | 3b | TPS16CC-3bR | CCTCACTAGGGTTTTTCTCAAAGG | SEQ ID 206 |
| TPS16CC | 4 | TPS16CC-4F | GGTGGAAAGAATTAGACTTGGC | SEQ ID 207 |
| TPS16CC | 4 | TPS16CC-4R | ATGTCCCACCTGAGAATTGC | SEQ ID 208 |
| TPS16CC | 5 | TPS16CC-5F | ACTTAGTCCAGATTATTTGAAGACATATT | SEQ ID 209 |
| TPS16CC | 5 | TPS16CC-5R | TGCATAGTGAAGTTTGTATCTCTCT | SEQ ID 210 |
| TPS16CC | 6 | TPS16CC-6F | TTGAGACCTCTTTTGTTGGAATGC | SEQ ID 211 |
| TPS16CC | 6 | TPS16CC-6R | AAAATCTTTGGTTGTGTAGAGAGC | SEQ ID 212 |
| TPS16CC | 7a | TPS16CC-7aF | ATGGTGTATCGGAACAAGAGG | SEQ ID 213 |
| TPS16CC | 7a | TPS16CC-7aR | AACGCAGCAACACTGTCC | SEQ ID 214 |
| TPS16CC | 7b | TPS16CC-7bF | AAGTGCTTGACATTATCTACAAAGAAGG | SEQ ID 215 |
| TPS16CC | 7b | TPS16CC-7bR | TAATGGGATGGGATCTATAAGCAACGC | SEQ ID 216 |
| TPS17JL | 1a | TPS17JL-1aF | ATGGCTTTTCACCAATTTGCTCC | SEQ ID 217 |
| TPS17JL | 1a | TPS17JL-1aR | TCGTAGAACTAGGGTTATCGACC | SEQ ID 218 |
| TPS17JL | 1b | TPS17JL-1bF | TTTGCTCCATCATCATCCCTCCC | SEQ ID 219 |
| TPS17JL | 1b | TPS17JL-1bR | GAGGTCCATAGTTGGCTGATCTTCG | SEQ ID 220 |
| TPS17JL | 2 | TPS17JL-2F | TTGAAGAAAGAAGTGACAAGAATGG | SEQ ID 221 |
| TPS17JL | 2 | TPS17JL-2R | GGTAAGATATTCCAAGCCTTTGC | SEQ ID 222 |
| TPS17JL | 4a | TPS17JL-4aF | TACATGGAGAAAATGGAGAATGAGG | SEQ ID 223 |
| TPS17JL | 4a | TPS17JL-4aR | TGAAGCGGAAGCTCGAAAGC | SEQ ID 224 |
| TPS17JL | 4b | TPS17JL-4bF | ATCACGCTTTCGAGCTTCCG | SEQ ID 225 |
| TPS17JL | 4b | TPS17JL-4bR | AAATCTTCTTGGTGTGTTGATTGC | SEQ ID 226 |
| TPS17JL | 5 | TPS17JL-5F | TTGCTAGAGATAGATTGATGGAAGC | SEQ ID 227 |
| TPS17JL | 5 | TPS17JL-5R | AAAGCTCTAATTCCTCCAAAGTTCC | SEQ ID 228 |
| TPS17JL | 6 | TPS17JL-6F | GTTACCAGATTACATGAAGATGCC | SEQ ID 229 |
| TPS17JL | 6 | TPS17JL-6R | ACTAATACATCGAACCCCATCTC | SEQ ID 230 |
| TPS17JL | 7 | TPS17JL-7F | AGTTGGGTTGGCTTTCAATAGG | SEQ ID 231 |
| TPS17JL | 7 | TPS17JL-7R | ATGTTCCTAAATCATCTGCAAGC | SEQ ID 232 |
| TPS17JL | 8 | TPS17JL-8F | TAGAGGCGACGTTCCTAAATCG | SEQ ID 233 |
| TPS17JL | 8 | TPS17JL-8R | TGCTGTTCTAGCCATATTTTTGC | SEQ ID 234 |
| TPS18JL | 1 | TPS18JL-1F | TCTAGTCAAGTGTTAGCTTCATCTC | SEQ ID 235 |
| TPS18JL | 1 | TPS18JL-1R | TGTTGTTGGTCGAATGATGTTTTG | SEQ ID 236 |
| TPS18JL | 2 | TPS18JL-2F | AAGAAGTTGTTAGGAAAGAGATATTCC | SEQ ID 237 |
| TPS18JL | 2 | TPS18JL-2R | AGATGAAATGTTAAATCCATGTTGTCG | SEQ ID 238 |
| TPS18JL | 3a | TPS18JL-3aF | TGAGAGTGGTAAGTTTAAGGAAAGC | SEQ ID 239 |
| TPS18JL | 3a | TPS18JL-3aR | GGTGGTAGTGAAAGCAAGAGC | SEQ ID 240 |
| TPS18JL | 3b | TPS18JL-3bF | GGCTCTTGCTTTCACTACCACC | SEQ ID 241 |
| TPS18JL | 3b | TPS18JL-3bR | CCTCACTAGGGTTTTTCTCAAAGG | SEQ ID 242 |
| TPS18JL | 3c | TPS18JL-3cF | TGAGAAAAACCCTAGTGAGGC | SEQ ID 243 |
| TPS18JL | 3c | TPS18JL-3cR | TTGTAGTAGATTGAAGTCCAACTTTGC | SEQ ID 244 |
| TPS18JL | 4 | TPS18JL-4F | TTAGACTTGGCAAACAAACTACC | SEQ ID 245 |
| TPS18JL | 4 | TPS18JL-4R | TATGTCCCACCTGAGAATTGC | SEQ ID 246 |
| TPS18JL | 5 | TPS18JL-5F | ACTTAGTCCAGATTATTTGAAGACATATTA | SEQ ID 247 |
| TPS18JL | 5 | TPS18JL-5R | TTGCATAGTGAAGTTTGTATCTCTCT | SEQ ID 248 |
| TPS18JL | 6a | TPS18JL-6aF | TTCCATGAAGCACAATGGTTGA | SEQ ID 249 |
| TPS18JL | 6a | TPS18JL-6aR | GCATTCCAACAAAAGAGGTCTCA | SEQ ID 250 |
| TPS18JL | 6b | TPS18JL-6bF | GGTTACCCAATGTTGATTGAGACC | SEQ ID 251 |
| TPS18JL | b | TPS18JL-6bR | GGTTGTGTAGAGAGCCATTCAAATA | SEQ ID 252 |
| TPS18JL | 7 | TPS18JL-7F | TGGTGTATCGGAACAAGAGGC | SEQ ID 253 |
| TPS18JL | 7 | TPS18JL-7R | CAACGCAGCAACACTGTCC | SEQ ID 254 |
| TPS18VF | 1 | TPS18VF-1F | ACGCATCTTTTCGTCCCTTT | SEQ ID 255 |
| TPS18VF | 1 | TPS18VF-1R | TGAGATCACCGTTACTCCTGATA | SEQ ID 256 |
| TPS18VF | 2 | TPS18VF-2F | AGCAGACCCATTTGATGAAGG | SEQ ID 257 |
| TPS18VF | 2 | TPS18VF-2R | CAGCAGGAACGAAGAGACCG | SEQ ID 258 |
| TPS18VF | 3a | TPS18VF-3aF | TCGACACAAGGCTAAGAGAGG | SEQ ID 259 |
| TPS18VF | 3a | TPS18VF-3aR | GTCCTTGGCTGTGAACTTGG | SEQ ID 260 |
| TPS18VF | 3b | TPS18VF-3bF | CAAGTTCACAGCCAAGGACC | SEQ ID 261 |
| TPS18VF | 3b | TPS18VF-3bR | TTATCAATTTCCATTCTATGCAGGG | SEQ ID 262 |
| TPS18VF | 4 | TPS18VF-4F | GGTGGCGAGACATTGGTTTAGC | SEQ ID 263 |
| TPS18VF | 4 | TPS18VF-4R | ATGGATTTTGTAAGCGCAACCC | SEQ ID 264 |
| TPS18VF | 5 | TPS18VF-5F | TGCTATACAAAAACTTCCAGACTCC | SEQ ID 265 |
| TPS18VF | 5 | TPS18VF-5R | TGTAAAGGGCTCCATCCACG | SEQ ID 266 |
| TPS18VF | 6a | TPS18VF-6aF | GGGCAAGTTTGTGCGAAGC | SEQ ID 267 |
| TPS18VF | 6a | TPS18VF-6aR | TGGCACTACCAAAGTCATCCC | SEQ ID 268 |
| TPS18VF | 6b | TPS18VF-6bF | ATGGTGTGGTCAGCTCAGG | SEQ ID 269 |
| TPS18VF | 6b | TPS18VF-6bR | TGGCACTACCAAAGTCATCCC | SEQ ID 270 |
| TPS18VF | 7a | TPS18VF-7aF | AGAATCAAGAAGGACATGACGG | SEQ ID 271 |
| TPS18VF | 7a | TPS18VF-7aR | TTTGTTGAGGCACTTCCATGC | SEQ ID 272 |
| TPS18VF | 7b | TPS18VF-7bF | AAGTGCCTCAACAAAGAATGC | SEQ ID 273 |
| TPS18VF | 7b | TPS18VF-7bR | GTTCTTCCAAGTGGGGTAGG | SEQ ID 274 |
| TPS19BL | 1 | TPS19BL-1F | ATGGCGTTGTCAATAATGTCTTCTTACG | SEQ ID 275 |
| TPS19BL | 1 | TPS19BL-1R | GAGAACTCGAAAGTGATGAGGAGG | SEQ ID 276 |
| TPS19BL | 2a | TPS19BL-2aF | AGCAGACCCATTTGATGAAGG | SEQ ID 277 |
| TPS19BL | 2a | TPS19BL-2aR | AGCAGGAACGAAGTGACCG | SEQ ID 278 |
| TPS19BL | 2b | TPS19BL-2bF | GATAAATACAGGGACGTTTTAAGAAAAGC | SEQ ID 279 |
| TPS19BL | 2b | TPS19BL-2bR | CCTCGAAGATATAGTCAATTCCTAGC | SEQ ID 280 |
| TPS19BL | 3 | TPS19BL-3F | ATATGAAGCCTCCCATCTATGC | SEQ ID 281 |
| TPS19BL | 3 | TPS19BL-3R | CCATTCTCACTGGGATAGACACC | SEQ ID 282 |
| TPS19BL | 4 | TPS19BL-4F | GGTGGCGAGACATTGGTTTAGC | SEQ ID 283 |
| TPS19BL | 4 | TPS19BL-4R | ATGGATTTTGTAAGCGCAACCC | SEQ ID 284 |
| TPS19BL | 5a | TPS19BL-5aF | AGAAAAACTTCCAGACTCCATGA | SEQ ID 285 |
| TPS19BL | 5a | TPS19BL-5aR | GCTCCATCCACGCTTTTGAT | SEQ ID 286 |
| TPS19BL | 5b | TPS19BL5bF | TCAATGAGTCTAGCCATACGATCT | SEQ ID 287 |
| TPS19BL | 5b | TPS19BL-5bR | TGTAAAGGGCTCCATCCACG | SEQ ID 288 |
| TPS19BL | 6 | TPS19BL-6F | GGGCAAGTTTGTGCGAAGC | SEQ ID 289 |
| TPS19BL | 6 | TPS19BL-6R | TGGCACTACCAAAGTCATCCC | SEQ ID 290 |
| TPS19BL | 7a | TPS19BL-7aF | GGACATGACGGATCTTATGTGG | SEQ ID 291 |
| TPS19BL | 7a | TPS19BL-7aR | TTGTTGAGGCACTTCCATGC | SEQ ID 292 |
| TPS19BL | 7b | TPS19BL-7bF | ATCCAGCGTTTCCACCACC | SEQ ID 293 |
| TPS19BL | 7b | TPS19BL-7bR | GTTCTTCCAAGTGGGGTAGGC | SEQ ID 294 |
| TPS1JL | 1 | TPS1JL-1F | ACCAATTTGCTTCATCATCATCCC | SEQ ID 295 |
| TPS1JL | 1 | TPS1JL-1R | AAATGGGAGGTCCATAGTTGGC | SEQ ID 296 |
| TPS1JL | 2 | TPS1JL-2F | TGAAAAGGATGCTAATTGGAGTGG | SEQ ID 297 |
| TPS1JL | 2 | TPS1JL-2R | CGTAGAAGCCTAAATTGGAGAGC | SEQ ID 298 |
| TPS1JL | 3 | TPS1JL-3F | ATCACAAGACAGGAGAGTTCAAGGC | SEQ ID 299 |
| TPS1JL | 3 | TPS1JL-3R | ATTCTCCATTGAAGTGGCATCTCC | SEQ ID 300 |
| TPS1JL | 4a | TPS1JL-4aF | ACTTGGAGAGAAATTGCCTTTCG | SEQ ID 301 |
| TPS1JL | 4a | TPS1JL-4aR | TCCAATGTTCCATAAATGTCATGC | SEQ ID 302 |
| TPS1JL | 4b | TPS1JL-4bF | GCAAGTTGGAGTAAGATTTGAGC | SEQ ID 303 |
| TPS1JL | 4b | TPS1JL-4bR | ATTAGTGAAAAGTTGTAGTTCCTCC | SEQ ID 304 |
| TPS1JL | 5 | TPS1JL-5F | TAAGTTACCAGATTATATGAAGACAGC | SEQ ID 305 |
| TPS1JL | 5 | TPS1JL-5R | TTTGTGAAATTGTATGTAAAGTAGAAAGC | SEQ ID 306 |
| TPS1JL | 6 | TPS1JL-6F | AGAATGGATGGTTGTCTGTGGG | SEQ ID 307 |
| TPS1JL | 6 | TPS1JL-6R | GGCGAACTATGTTAGGATGACC | SEQ ID 308 |
| TPS1JL | 7 | TPS1JL-7F | CAATTCAATGTTATATGCACGATACTGG | SEQ ID 309 |
| TPS1JL | 7 | TPS1JL-7R | CTGAGAAGCATGTCCATCGCC | SEQ ID 310 |
| TPS20CT | 1 | TPS20CT-1F | ATTCAAGTCTTAGCTTCATCTCAATTA | SEQ ID 311 |
| TPS20CT | 1 | TPS20CT-1R | CGATCACCCCAAATAGAAGGGT | SEQ ID 312 |
| TPS20CT | 2 | TPS20CT-2F | TTTGAGAGTGAAATTGAGAAATTGTTGG | SEQ ID 313 |
| TPS20CT | 2 | TPS20CT-2R | TAAATCCATGTTGTCTTAATAATCTAAACC | SEQ ID 314 |
| TPS20CT | 3a | TPS20CT-3aF | GCCTAATAACCGATGTTCCAGG | SEQ ID 315 |
| TPS20CT | 3a | TPS20CT-3aR | GAGATGTAAAACCTAGCATGAAGCC | SEQ ID 316 |
| TPS20CT | 3b | TPS20CT-3bF | AGACCATAGAGAGGCTTCATGC | SEQ ID 317 |
| TPS20CT | 3b | TPS20CT-3bR | CGTAATTTCACTAAGTTCCTTTTTGTGG | SEQ ID 318 |
| TPS20CT | 4 | TPS20CT-4F | GGTGGAAGGAGCATGAGTTTGC | SEQ ID 319 |
| TPS20CT | 4 | TPS20CT-4R | ACCTTTGAATTGCTTTGGTAAGAAGC | SEQ ID 320 |
| TPS20CT | 6 | TPS20CT-6F | GAGTCTCTTATGTTTCTTCTGGTAACG | SEQ ID 321 |
| TPS20CT | 6 | TPS20CT-6R | CATGAACCTAGAAAGGAGAGTAGAAGC | SEQ ID 322 |
| TPS20CT | 7a | TPS20CT-7aF | TGAGCAAAAGAGAAATCACATACC | SEQ ID 323 |
| TPS20CT | 7a | TPS20CT-7aR | TTTCTTTCCAGTGGGTGTCC | SEQ ID 324 |
| TPS20CT | 7b | TPS20CT-7bF | CCAGCAGTTGTGCCCTTTCC | SEQ ID 325 |
| TPS20CT | 7b | TPS20CT-7bR | CAATGCTTTCTTTGAGCACTTTTCC | SEQ ID 326 |
| TPS20JL | 1 | TPS20JL-1F | TCAAGTCTTAGCTTCATCTCAATTATGTG | SEQ ID 327 |
| TPS20JL | 1 | TPS20JL-1R | GATCACCCCAAATAGAAGGGT | SEQ ID 328 |
| TPS20JL | 2 | TPS20JL-2F | TTCGAGAGTGAAATCGAGAAATTATTGG | SEQ ID 329 |
| TPS20JL | 2 | TPS20JL-2R | ATCCACTTTGTCTTAATAGTCTAAACCG | SEQ ID 330 |
| TPS20JL | 3a | TPS20JL-3aF | CTTGCTTTCACAACCACTCACC | SEQ ID 331 |
| TPS20JL | 3a | TPS20JL-3aR | CCTCTCTATGGTCTTTCTTAGAGGC | SEQ ID 332 |
| TPS20JL | 3b | TPS20JL-3bF | ATAACCGATGTTTCAGGTTTGC | SEQ ID 333 |
| TPS20JL | 3b | TPS20JL-3bR | TAAGGTGAGTGGTTGTGAAAGC | SEQ ID 334 |
| TPS20JL | 4 | TPS20JL-4F | GTGTATATTATGAACCCAAATACTCTCG | SEQ ID 335 |
| TPS20JL | 4 | TPS20JL-4R | CCTTTGAATTGCTTTGGTAAGAAGC | SEQ ID 336 |
| TPS20JL | 6 | TPS20JL-6F | GAACTATTGGAGGTTATTTTGAAGAAGC | SEQ ID 337 |
| TPS20JL | 6 | TPS20JL-6R | GAGTGGAAGCTGAAACAATCTTAGGG | SEQ ID 338 |
| TPS20JL | 7 | TPS20JL-7F | TGAGCAAGAGAGAAATCACATACC | SEQ ID 339 |
| TPS20JL | 7 | TPS20JL-7R | AGAACACGAAGTAAGATAGGAAAAGG | SEQ ID 340 |
| TPS23JL | 1 | TPS23JL-1F | CACACAAATCTTAGTATCATTATCTTCAAA | SEQ ID 341 |
| TPS23JL | 1 | TPS23JL-1R | CAAATCTTGTTGTGAAATGTTGTAATGT | SEQ ID 342 |
| TPS23JL | 2 | TPS23JL-2F | TGTCAACAACAAAAGGTTGAGG | SEQ ID 343 |
| TPS23JL | 2 | TPS23JL-2R | TCAAAATGATAAGACAATCCCAAACG | SEQ ID 344 |
| TPS23JL | 3a | TPS23JL-3aF | GAACACCATGAAGACGATGATCC | SEQ ID 345 |
| TPS23JL | 3a | TPS23JL-3aR | TGCTGACTTCACTAAGCTCC | SEQ ID 346 |
| TPS23JL | 3b | TPS23JL-3bF | TGCTTGGTAAGTGATACCCTTGG | SEQ ID 347 |
| TPS23JL | 3b | TPS23JL-3bR | AGGGCCTCTCTAGGGCTCG | SEQ ID 348 |
| TPS23JL | 4 | TPS23JL-4F | TGCAAGAGATAGGATTGTGGAATTG | SEQ ID 349 |
| TPS23JL | 4 | TPS23JL-4R | ACTCAAGTTCTTCAAGTGTACCATA | SEQ ID 350 |
| TPS23JL | 5 | TPS23JL-5F | TTGTTATGAAGAGTTTGAGCAACTG | SEQ ID 351 |
| TPS23JL | 5 | TPS23JL-5R | ACTCTGTATGTTTCTTCTTTTTCTAGCAG | SEQ ID 352 |
| TPS23JL | 6 | TPS23JL-6F | TTTGAATGAAGCTCGATGTTTGC | SEQ ID 353 |
| TPS23JL | 6 | TPS23JL-6R | CCATTAGCCTACAAATAGTAACAGATGC | SEQ ID 354 |
| TPS23JL | 7a | TPS23JL-7aF | GCGTTTGTGAAGAGGAAGCC | SEQ ID 355 |
| TPS23JL | 7a | TPS23JL-7aR | ACCCTTGAAAAGTTAAGAGCACG | SEQ ID 356 |
| TPS23JL | 7b | TPS23JL-7bF | AGAAATAAATGAAGAGTTTTTGAAGCC | SEQ ID 357 |
| TPS23JL | 7b | TPS23JL-7bR | TGGATGAATAAGCAAAGCAGC | SEQ ID 358 |
| TPS24JL | 10a | TPS24JL-10aF | GCTCAAGTCTATGTTGAAGGAAGC | SEQ ID 359 |
| TPS24JL | 10a | TPS24JL-10aR | AACATCCTCAGAAAGCATAGGTCC | SEQ ID 360 |
| TPS24JL | 10b | TPS24JL-10bF | TCAATGGTTGAAATACGAGACCG | SEQ ID 361 |
| TPS24JL | 10b | TPS24JL-10bR | ATCGTTGAGAAGTCGTCCG | SEQ ID 362 |
| TPS24JL | 11 | TPS24JL-11F | CGCGTGGTTTTGGCTCTGG | SEQ ID 363 |
| TPS24JL | 11 | TPS24JL-11R | GGTGAATCCGTCGTCATTGGC | SEQ ID 364 |
| TPS24JL | 1 | TPS24JL-1F | TGCATTCCAACATTAGGTGCTC | SEQ ID 365 |
| TPS24JL | 1 | TPS24JL-1R | TGCTTTTCCTTGCACCATTTAGT | SEQ ID 366 |
| TPS24JL | 2a | TPS24JL-2aF | TTATGACACTGCCTGGGTGG | SEQ ID 367 |
| TPS24JL | 2a | TPS24JL-2aR | GAGGAAGACCCCATGAACCG | SEQ ID 368 |
| TPS24JL | 2b | TPS24JL-2bF | AGAGAATCAGCACTCTGACGG | SEQ ID 369 |
| TPS24JL | 2b | TPS24JL-2bR | CACCCCATCGCTTCATTGC | SEQ ID 370 |
| TPS24JL | 4 | TPS24JL-4F | TCAGGAAGCTACTCAGAGGG | SEQ ID 371 |
| TPS24JL | 4 | TPS24JL-4R | ACAGAGAACCATTTTTCCTTTGG | SEQ ID 372 |
| TPS24JL | 5 | TPS24JL-5F | TATGCTCGTCTCTCAATGGTGG | SEQ ID 373 |
| TPS24JL | 5 | TPS24JL-5R | AGAACACACTCCTCTCCTTGC | SEQ ID 374 |
| TPS24JL | 6 | TPS24JL-6F | TCCGAGATCATCATACACCCA | SEQ ID 375 |
| TPS24JL | 6 | TPS24JL-6R | TCTCCCCTAGTTGAAATGCTGT | SEQ ID 376 |
| TPS24JL | 7 | TPS24JL-7F | GAAAACTTCATATTGTTCCTCAAATGTTCG | SEQ ID 377 |
| TPS24JL | 7 | TPS24JL-7R | GAGTTCTTCACGGTGTATTGATTGG | SEQ ID 378 |
| TPS24JL | 8a | TPS24JL-8aF | GTGGGTTGTAGAGAATAGGTTGG | SEQ ID 379 |
| TPS24JL | 8a | TPS24JL-8aR | GCGGGCATCAGATAATTCAGG | SEQ ID 380 |
| TPS24JL | 8b | TPS24JL-8bF | CCTGAATTATCTGATGCCCGC | SEQ ID 381 |
| TPS24JL | 8b | TPS24JL-8bR | CTAGTTCCTCTTCTGAACCTCC | SEQ ID 382 |
| TPS24JL | 9 | TPS24JL-9F | CAGTGTTGATTGTTTGTCGGAGC | SEQ ID 383 |
| TPS24JL | 9 | TPS24JL-9R | TTGTCACACTACGTCCTTGC | SEQ ID 384 |
| TPS2FN | 1a | TPS2FN-1aF | AAGATCAGCCAACTATGATCCTCCC | SEQ ID 385 |
| TPS2FN | 1a | TPS2FN-1aR | ATGGAAGAGACTGAATGAAATCAAAAGACC | SEQ ID 386 |
| TPS2FN | 1b | TPS2FN-1bF | TTAGAAGATCAGCCAACTATGATCC | SEQ ID 387 |
| TPS2FN | 1b | TPS2FN-1bR | GAATGAAATCAAAAGACCAAATGGG | SEQ ID 388 |
| TPS2FN | 2 | TPS2FN-2F | AAAGAAGAAGTGAAAAAGATGTTAGTTGG | SEQ ID 389 |
| TPS2FN | 2 | TPS2FN-2R | AAAAGCCTAAATTCGAGAGCAGTGG | SEQ ID 390 |
| TPS2FN | 3a | TPS2FN-3aF | GAGACGGGAAAGTTCAAAGCG | SEQ ID 391 |
| TPS2FN | 3a | TPS2FN-3aR | TGAAGTGGCATCTCCAAGGC | SEQ ID 392 |
| TPS2FN | 3b | TPS2FN-3bF | CGCAAATTCAAGCAAAGTGC | SEQ ID 394 |
| TPS2FN | 3b | TPS2FN-3bF | GCTTCATTCTATGTGAAAAATGGCG | SEQ ID 393 |
| TPS2FN | 4a | TPS2FN-4aF | AAATGGTTTATGCTAGAGATAGATTGG | SEQ ID 395 |
| TPS2FN | 4a | TPS2FN-4aR | CTTGCAGATATTCTCCTAAAGTGGC | SEQ ID 396 |
| TPS2FN | 4b | TPS2FN-4bF | AGAGGCTTTTCTATGGCAGG | SEQ ID 397 |
| TPS2FN | 4b | TPS2FN-4bR | AAGCTCTAATTCTTCCAATGTTCC | SEQ ID 398 |
| TPS2FN | 5a | TPS2FN-5aF | TGCCTTTCTTTACTTTATTTAACACCG | SEQ ID 399 |
| TPS2FN | 5a | TPS2FN-5aR | GCTCTTCTAACACATCATACGCC | SEQ ID 400 |
| TPS2FN | 5b | TPS2FN-5bF | ACACCGTAAATGAAATGGCG | SEQ ID 401 |
| TPS2FN | 5b | TPS2FN-5bR | CGAGTTCTTGAGGTATTCAACGC | SEQ ID 402 |
| TPS2FN | 6a | TPS2FN-6aF | TGGGCAGAGTTATGTAGATGC | SEQ ID 403 |
| TPS2FN | 6a | TPS2FN-6aR | TCCTATTGAAAGCGAGGCG | SEQ ID 404 |
| TPS2FN | 6b | TPS2FN-6bF | ACGCCTCGCTTTCAATAGG | SEQ ID 405 |
| TPS2FN | 6b | TPS2FN-6bR | CTGCAAGTCGTAACATCAAGG | SEQ ID 406 |
| TPS2FN | 6c | TPS2FN-6cF | TGGAAGAGGCAAAATGGTTTTATAGC | SEQ ID 407 |
| TPS2FN | 6c | TPS2FN-6cR | GATCATCTGCAAGTCGTAACATCAAGG | SEQ ID 408 |
| TPS2FN | 7a | TPS2FN-7aF | AAAGAGGTGACATTCTTAAATCGG | SEQ ID 409 |
| TPS2FN | 7a | TPS2FN-7aR | AGTTATATTCATCTTCATCATTCATCTCC | SEQ ID 410 |
| TPS2FN | 7b | TPS2FN-7bF | GGTGTTTCTGAAGATGAAGCTCG | SEQ ID 411 |
| TPS2FN | 7b | TPS2FN-7bR | CTGAAATACGTTTCCTTGAATGGC | SEQ ID 412 |
| TPS30JL | 2 | TPS30JL-2F | TCTCGAAGATCAGCAAACTATCAACC | SEQ ID 413 |
| TPS30JL | 2 | TPS30JL-2R | ACATAATCAAATTGCCAAAGTGGG | SEQ ID 414 |
| TPS30JL | 4a | TPS30JL-4aF | AGACTTGGAATCTCTTATCACTTTGAG | SEQ ID 415 |
| TPS30JL | 4a | TPS30JL-4aR | GGCATACACATTATTTTTGTTGGTGTT | SEQ ID 416 |
| TPS30JL | 4b | TPS30JL-4bF | GTACAACACCAACAAAAATAATGTGT | SEQ ID 417 |
| TPS30JL | 4b | TPS30JL-4bR | TCGTAGGAGTCTAAATTCAAGAGAA | SEQ ID 418 |
| TPS30JL | 5 | TPS30JL-5F | GAGGCAATTTTATGGTGTGTACC | SEQ ID 419 |
| TPS30JL | 5 | TPS30JL-5R | TTGTTTTGTGTCTTGTCTCTTCC | SEQ ID 420 |
| TPS30JL | 6 | TPS30JL-6F | TGGGAAAGCACTGATATGGG | SEQ ID 421 |
| TPS30JL | 6 | TPS30JL-6R | AGCTCCAATTCATCTAGTGTACC | SEQ ID 422 |
| TPS30JL | 7 | TPS30JL-7F | TGGGACATTAGTGCTATGGATGG | SEQ ID 423 |
| TPS30JL | 7 | TPS30JL-7R | TCTGAAGGAATTTTATTATATGGAGGC | SEQ ID 424 |
| TPS30JL | 8 | TPS30JL-8F | AGATTTGAAGAGTACATTGAGAATGC | SEQ ID 425 |
| TPS30JL | 8 | TPS30JL-8R | CGATGAATGTCGTATTATGGTAGGG | SEQ ID 426 |
| TPS30JL | 9 | TPS30JL-9F | AGAGGAAGCTCGTCAACGTAT | SEQ ID 427 |
| TPS30JL | 9 | TPS30JL-9R | ATGTGCCATTCTACCAAGGTT | SEQ ID 428 |
| TPS30-likeJL | 1 | TPS30-likeJL-1F | GACTTGGAATCTCTTACCACTTTGA | SEQ ID 429 |
| TPS30-likeJL | 1 | TPS30-likeJL-1R | ACCATGTTGTCTTAGGAGTCTAAAT | SEQ ID 430 |
| TPS30-likeJL | 2 | TPS30-likeJL-2F | GAATAGTTCTCGAAGATCAGCAAAC | SEQ ID 431 |
| TPS30-likeJL | 2 | TPS30-likeJL-2R | CAAATTGCCAAAGTGGGGGT | SEQ ID 432 |
| TPS30-likeJL | 3 | TPS30-likeJL-3F | GCAAAGACTTGGAATCTCTTACCA | SEQ ID 433 |
| TPS30-likeJL | 3 | TPS30-likeJL-3R | GAGACACCGGATAACCATGTTG | SEQ ID 434 |
| TPS30-likeJL | 4 | TPS30-likeJL-4F | GAGGCAATTTTATGGTGTCTTCC | SEQ ID 435 |
| TPS30-likeJL | 4 | TPS30-likeJL-4R | TGTTGTGTGTCTTGTCTCTTCC | SEQ ID 436 |
| TPS30-likeJL | 5 | TPS30-likeJL-5F | GTGGGAAAGCACTGGTATGG | SEQ ID 437 |
| TPS30-likeJL | 5 | TPS30-likeJL-5R | AGCTCCAATTCATCTAGTGTACC | SEQ ID 438 |
| TPS30-likeJL | 6 | TPS30-likeJL-6F | TTAGTGCTATGGATGGGCTCC | SEQ ID 439 |
| TPS30-likeJL | 6 | TPS30-likeJL-6R | CGATTTCTGAAGGACTTTTATTATATGG | SEQ ID 440 |
| TPS30-likeJL | 7 | TPS30-likeJL-7F | AGAGAAGCAAGATGGTATTATGATGG | SEQ ID 441 |
| TPS30-likeJL | 7 | TPS30-likeJL-7R | CGATGAATGTCGTATTATGGTAGGG | SEQ ID 442 |
| TPS30-likeJL | 8a | TPS30-likeJL-8aF | TGGCGTGTCTGAAGAAGAAGC | SEQ ID 443 |
| TPS30-likeJL | 8a | TPS30-likeJL-8aR | AACATTGGAGAGTAGTCATCACC | SEQ ID 444 |
| TPS30-likeJL | 8b | TPS30-likeJL-8bF | CATGATGGTGATGACTACTCTCC | SEQ ID 445 |
| TPS30-likeJL | 8b | TPS30-likeJL-8bR | AGTACATGATCTTTTGTTTGGCG | SEQ ID 446 |
| TPS32JL | 1 | TPS32JL-1F | TGCAAAGAAGAACGAGTGAAGG | SEQ ID 447 |
| TPS32JL | 1 | TPS32JL-1R | TGTTTGTAGTAGTTTAGATCATGTTTTTCC | SEQ ID 448 |
| TPS32JL | 2 | TPS32JL-2F | ATAACCGATGTTTCGGGTTTGC | SEQ ID 449 |
| TPS32JL | 2 | TPS32JL-2R | CCTCTCTATGGTCTTTCTTAATGGC | SEQ ID 450 |
| TPS32JL | 3 | TPS32JL-3F | GAAAGCTAACAACCAAAATCTCTGC | SEQ ID 451 |
| TPS32JL | 3 | TPS32JL-3R | TTGCATGGCTTGGGTAAGAAGC | SEQ ID 452 |
| TPS32JL | 5 | TPS32JL-5F | AGAAGAAGCTCGATGGTTGAACG | SEQ ID 453 |
| TPS32JL | 5 | TPS32JL-5R | CATGAACCTAGCAAGTAGAGTGG | SEQ ID 454 |
| TPS32JL | 6 | TPS32JL-6F | GCCAACTGTTATGCCTTTCCC | SEQ ID 455 |
| TPS32JL | 6 | TPS32JL-6R | GGGATTGGATCTATAAGTAAAGCAGC | SEQ ID 456 |
| TPS33JL | 1 | TPS33JL-1F | ATGCTACCCCATCCAATGTGC | SEQ ID 457 |
| TPS33JL | 1 | TPS33JL-1R | AATATAATCGAAAGACCAAATGGAGGGC | SEQ ID 458 |
| TPS33JL | 2 | TPS33JL-2F | ATTAGTTGAGATGGAAAACTCTTTAGC | SEQ ID 459 |
| TPS33JL | 2 | TPS33JL-2R | ATAGCCATGTTGACGTAGAAGCC | SEQ ID 460 |
| TPS33JL | 3 | TPS33JL-3F | AGCTCCCACTTCATCGGAGG | SEQ ID 461 |
| TPS33JL | 3 | TPS33JL-3R | TTTGGCTAACTCAAGCAACATAGG | SEQ ID 462 |
| TPS33JL | 4a | TPS33JL-4aF | AGACATACTAAACTTGGAGAGAAATTGA | SEQ ID 463 |
| TPS33JL | 4a | TPS33JL-4aR | TATTCCATAAGAAACATTCCATCAATCG | SEQ ID 464 |
| TPS33JL | 4b | TPS33JL-4bF | GGAGACATACTAAACTTGGAGAGA | SEQ ID 465 |
| TPS33JL | 4b | TPS33JL-4bR | GCTTTGGTGAAAAGCTCTAATTCAT | SEQ ID 466 |
| TPS33JL | 5 | TPS33JL-5F | AGTTACCAGAATACATGAAGATGCC | SEQ ID 467 |
| TPS33JL | 5 | TPS33JL-5R | TGAATGTTGATGGAGATTTCTTGG | SEQ ID 468 |
| TPS33JL | 6 | TPS33JL-6F | AAATGGTTGGATTTCAGTAGGAGC | SEQ ID 469 |
| TPS33JL | 6 | TPS33JL-6R | GAAAATCTCTTTAGTATTTGTAATTGTGCC | SEQ ID 470 |
| TPS33JL | 7 | TPS33JL-7F | AATTGAAAAGAGGTGATGCTCCG | SEQ ID 471 |
| TPS33JL | 7 | TPS33JL-7R | TTTGGATTGATTATCTTGAGAACTATGACC | SEQ ID 472 |
| TPS36JL | 1 | TPS36JL-1F | AAAGATCAACCAGCAGCAATCG | SEQ ID 473 |
| TPS36JL | 1 | TPS36JL-1R | GTGGGTTTGTAGTTTCCTGATCG | SEQ ID 474 |
| TPS36JL | 2 | TPS36JL-2F | AAGGAGAGTGAAAATCCTTTAGTTAAGC | SEQ ID 475 |
| TPS36JL | 2 | TPS36JL-2R | GAGTTTGAAATGAAGAGCAGTGGC | SEQ ID 476 |
| TPS36JL | 3 | TPS36JL-3F | AATGCCTTCAAAAACGAGCAAAAGG | SEQ ID 477 |
| TPS36JL | 3 | TPS36JL-3R | GTCATCAAGTATTGTTTGAGATGTTTGG | SEQ ID 478 |
| TPS36JL | 5 | TPS36JL-5F | TGGCATTGAAATATGAGGCGG | SEQ ID 479 |
| TPS36JL | 5 | TPS36JL-5R | GCTGAAGCTCATCTAGTGTACC | SEQ ID 480 |
| TPS36JL | 6 | TPS36JL-6F | ATAAATGAACTGGATCAGCTACCCG | SEQ ID 481 |
| TPS36JL | 6 | TPS36JL-6R | GATGGTGTGAATCCCATTTTCTTTGAGG | SEQ ID 482 |
| TPS36JL | 7 | TPS36JL-7F | TTGGGGGATCTGTGTAAATGC | SEQ ID 483 |
| TPS36JL | 7 | TPS36JL-7R | CAGTTCCTGAGACACGTAAAATGG | SEQ ID 484 |
| TPS37FN | 1 | TPS37FN-1F | GCAGTGCATGGCTTTTCACC | SEQ ID 485 |
| TPS37FN | 1 | TPS37FN-1R | TCCAAATAGGGAGGGATGATGA | SEQ ID 486 |
| TPS37FN | 2 | TPS37FN-2F | ACAATGTACTGTGGTCGATAACCC | SEQ ID 487 |
| TPS37FN | 2 | TPS37FN-2R | AATGAAATCAAAAGACCAAATGGGAGG | SEQ ID 488 |
| TPS37FN | 3 | TPS37FN-3F | GAGAAAGACGTGAAAAGGATACTGG | SEQ ID 489 |
| TPS37FN | 3 | TPS37FN-3R | TCAAATCCATATTGGCGTAGAAGC | SEQ ID 490 |
| TPS37FN | 4 | TPS37FN-4F | AGCTTCATTCTATGGGAAAAAGGG | SEQ ID 491 |
| TPS37FN | 4 | TPS37FN-4R | TGAACCACCTAGCTTCCAACC | SEQ ID 492 |
| TPS37FN | 5 | TPS37FN-5F | GCAATCTAAACTTGGAGAAAAGAAAATGG | SEQ ID 493 |
| TPS37FN | 5 | TPS37FN-5R | TTTGTGAAAAGCTCTAATTCCTCC | SEQ ID 494 |
| TPS37FN | 7 | TPS37FN-7F | CATGCTTATTTTGCTTTCACAAATCCC | SEQ ID 495 |
| TPS37FN | 7 | TPS37FN-7R | TAGGTCATCTTCAAGTCGTAAAAGTATGG | SEQ ID 496 |
| TPS37FN | 8a | TPS37FN-8aF | GAGATGAAAAGAGGAGATGTTCCG | SEQ ID 497 |
| TPS37FN | 8a | TPS37FN-8aR | AAGCATGTCCATCACCATAAAGG | SEQ ID 498 |
| TPS37FN | 8b | TPS37FN-8bF | AAACATGGAAAGAGATGAATAAAGAAATGG | SEQ ID 499 |
| TPS37FN | 8b | TPS37FN-8bR | GAGAAGCATGTCCATCACCATAAAGG | SEQ ID 500 |
| TPS37JL | 1 | TPS37JL-1F | ATTCAAAGAACAACCGGAAGC | SEQ ID 501 |
| TPS37JL | 1 | TPS37JL-1R | AAAAATGAGCATCCCACAACG | SEQ ID 502 |
| TPS37JL | 2 | TPS37JL-2F | AAAGCTATGGGAAAAGAATCAATGAGC | SEQ ID 503 |
| TPS37JL | 2 | TPS37JL-2R | GGAGTTTGAAATGAAGAGCAGTGG | SEQ ID 504 |
| TPS37JL | 3 | TPS37JL-3F | GAGGCTTCATTCTATTCATTTAGGGG | SEQ ID 505 |
| TPS37JL | 3 | TPS37JL-3R | TCAACAGATTTAGTTTGACATTGCC | SEQ ID 506 |
| TPS37JL | 4 | TPS37JL-4F | TCTAGGTTGACAGAAAGGCTACC | SEQ ID 507 |
| TPS37JL | 4 | TPS37JL-4R | GTCCATGAGTGTTAGCAAGAGACC | SEQ ID 508 |
| TPS37JL | 5 | TPS37JL-5F | TATAAATGAATTGGATCAGCTACCCG | SEQ ID 509 |
| TPS37JL | 5 | TPS37JL-5R | TATAAAAAGCAACAAACAATATCTTCATGT | SEQ ID 510 |
| TPS37JL | 6a | TPS37JL-6aF | GTTGGGGGATCTGTGTAAATGC | SEQ ID 511 |
| TPS37JL | 6a | TPS37JL-6aR | AGTTGGATATTGAAGCAAGTGTTCC | SEQ ID 512 |
| TPS37JL | 6b | TPS37JL-6bF | TGTGTAAATGCTATATGGAGGAGGC | SEQ ID 513 |
| TPS37JL | 6b | TPS37JL-6bR | TCGAAAGACAGTTCCTGAGACACG | SEQ ID 514 |
| TPS37JL | 7 | TPS37JL-7F | TGCTACATGCGTGAAAAGGG | SEQ ID 515 |
| TPS37JL | 7 | TPS37JL-7R | AGTACCAAAGCCATCACCC | SEQ ID 516 |
| TPS38FN | 1 | TPS38FN-1F | TTAATATCATCATCACTACCTTGCATT | SEQ ID 517 |
| TPS38FN | 1 | TPS38FN-1R | GGACTAATAGATGTTTTTGGTGTGAA | SEQ ID 518 |
| TPS38FN | 2 | TPS38FN-2F | CAATGTACTGTGGTCAATAATAGTAGCC | SEQ ID 519 |
| TPS38FN | 2 | TPS38FN-2R | AATCGAAAGACCAAATCGGAGG | SEQ ID 520 |
| TPS38FN | 3 | TPS38FN-3F | ATAAGGGAGAATCCTATACAAGGC | SEQ ID 521 |
| TPS38FN | 3 | TPS38FN-3R | CCTAAATTCGAGAGCAATGGGG | SEQ ID 522 |
| TPS38FN | 4 | TPS38FN-4F | AGCATATTGGAGGAAGCTAGGG | SEQ ID 523 |
| TPS38FN | 4 | TPS38FN-4R | TCGATAAACCACTTGGCCTCC | SEQ ID 524 |
| TPS38FN | 5 | TPS38FN-5F | GATCTAAAACATTTGTCTAGGTGGTGG | SEQ ID 525 |
| TPS38FN | 5 | TPS38FN-5R | ATTCTCCTAAAGTAGCTGAAATCTGG | SEQ ID 526 |
| TPS38FN | 6 | TPS38FN-6F | TCGTGTTGTTGAGAGATGGG | SEQ ID 527 |
| TPS38FN | 6 | TPS38FN-6R | AAGTGTTTTTCTTTTAATACATCTAGTGCC | SEQ ID 528 |
| TPS38FN | 7 | TPS38FN-7F | TGGTTGGATTTCAGTAGGAGC | SEQ ID 529 |
| TPS38FN | 7 | TPS38FN-7R | ACGATCATGGCAGATTGACG | SEQ ID 530 |
| TPS38FN | 8 | TPS38FN-8F | CATAACGTATCTAAAGAGGAAGCTCG | SEQ ID 531 |
| TPS38FN | 8 | TPS38FN-8R | TGCAAAGTTTTTGGCATCATCAACC | SEQ ID 532 |
| TPS38JL | 1 | TPS38JL-1F | AAAGATCAACCAGCAGCAATCG | SEQ ID 533 |
| TPS38JL | 1 | TPS38JL-1R | GTGGGTTTGTAGTTTCCTGATCG | SEQ ID 534 |
| TPS38JL | 2a | TPS38JL-2aF | AAGCTATGGGAAAAGAATCAATGAGC | SEQ ID 535 |
| TPS38JL | 2a | TPS38JL-2aR | GAGTTTGAAATGAAGAGCAGTGG | SEQ ID 536 |
| TPS38JL | 2b | TPS38JL-2bF | AGGCTGAAAATCCTTTAGTTAAGC | SEQ ID 537 |
| TPS38JL | 2b | TPS38JL-2bR | GGACTGAATCCATATTGTCGAAGG | SEQ ID 538 |
| TPS38JL | 3 | TPS38JL-3F | TGCCTTCAAAAACGAGAAAAAGG | SEQ ID 539 |
| TPS38JL | 3 | TPS38JL-3R | AGTCTCTTGCTTCATCTAATATGGG | SEQ ID 540 |
| TPS38JL | 4 | TPS38JL-4F | TGCAGTGGCATTGAAATATGAGG | SEQ ID 541 |
| TPS38JL | 4 | TPS38JL-4R | GCTGAAGCTCATCTAGTGTACC | SEQ ID 542 |
| TPS38JL | 5 | TPS38JL-5F | ATAAATGAACTGGATCAGCTACCCG | SEQ ID 543 |
| TPS38JL | 5 | TPS38JL-5R | TATAAAAAGCAACAAACAATATCTTCATGT | SEQ ID 544 |
| TPS38JL | 6 | TPS38JL-6F | TGCTATATGGAGGAGGCAAAATGG | SEQ ID 545 |
| TPS38JL | 6 | TPS38JL-6R | TCGAAAAACAGTTCCTGAGACACG | SEQ ID 546 |
| TPS38JL | 7 | TPS38JL-7F | TGCTACATGCGTGAAAAGGG | SEQ ID 547 |
| TPS38JL | 7 | TPS38JL-7R | TGTGGTACATCTCCATTGCTCC | SEQ ID 548 |
| TPS39JL | 1 | TPS39JL-1F | TGGCTTTTCACCAATTTGCTCC | SEQ ID 549 |
| TPS39JL | 1 | TPS39JL-1R | AAATGGGAGGTCCATAGTTGGC | SEQ ID 550 |
| TPS39JL | 2 | TPS39JL-2F | TTGAAGAAAGAAGTGACAAGATGGC | SEQ ID 551 |
| TPS39JL | 2 | TPS39JL-2R | GGCGTAGAAGCCTAAATTCGC | SEQ ID 552 |
| TPS39JL | 3 | TPS39JL-3F | ACATGGAGAAAATGGAGAATGAGG | SEQ ID 553 |
| TPS39JL | 3 | TPS39JL-3R | CGCAACCTCGAAACAAGTCG | SEQ ID 554 |
| TPS39JL | 4 | TPS39JL-4F | AGGGGATTGTAAACTTGGTGG | SEQ ID 555 |
| TPS39JL | 4 | TPS39JL-4R | TTCTCTAAAATAGCTGAAATTCTCCTCG | SEQ ID 556 |
| TPS39JL | 5 | TPS39JL-5F | TGAGTTACCAGATTACATGAAGATGCC | SEQ ID 557 |
| TPS39JL | 5 | TPS39JL-5R | ACTAATACATCGAACCCCATCTCA | SEQ ID 558 |
| TPS39JL | 6 | TPS39JL-6F | ACAAGAAGCAAAATGGTATTATAGTGG | SEQ ID 559 |
| TPS39JL | 6 | TPS39JL-6R | TTTTGTTATAGGATTTGTGAAACAATAAGC | SEQ ID 560 |
| TPS39JL | 7 | TPS39JL-7F | ATAATGCTACCGAAGACGAAGC | SEQ ID 561 |
| TPS39JL | 7 | TPS39JL-7R | TCTGAGAACCATGTCCATCTCC | SEQ ID 562 |
| TPS3JL | 1 | TPS3JL-1F | AATCACTTTTGTAGATTTTTCACACC | SEQ ID 563 |
| TPS3JL | 1 | TPS3JL-1R | GTAATCGAAAGACCAAATCGGAGG | SEQ ID 564 |
| TPS3JL | 2 | TPS3JL-2F | AGAAAGAAGTGACAAGAATGCTCC | SEQ ID 565 |
| TPS3JL | 2 | TPS3JL-2R | AGCCATGTTGGCGTAGAAGC | SEQ ID 566 |
| TPS3JL | 3a | TPS3JL-3aF | TGCTTTCAAGGATAAGAGAGGG | SEQ ID 567 |
| TPS3JL | 3a | TPS3JL-3aR | TCTTCCTCATTCTCCATTTTCTCC | SEQ ID 568 |
| TPS3JL | 3b | TPS3JL-3bF | AATACATGGAGAAAATGGAGAATGAGG | SEQ ID 569 |
| TPS3JL | 3b | TPS3JL-3bR | CGCAAACTCGAACAAAGTCG | SEQ ID 570 |
| TPS3JL | 4 | TPS3JL-4F | TTTGCTAGAGATAGATTGATGGAAGC | SEQ ID 571 |
| TPS3JL | 4 | TPS3JL-4R | AAAAGCTCTAATTCCTCCAAAGTTCC | SEQ ID 572 |
| TPS3JL | 5 | TPS3JL-5F | ATCAATGAGTTACCAGATTACATGAAG | SEQ ID 573 |
| TPS3JL | 5 | TPS3JL-5R | ACTAATACATCGAACCCCATCTCAT | SEQ ID 574 |
| TPS3JL | 6 | TPS3JL-6F | ATACACTGAGTTGGGTTGGC | SEQ ID 575 |
| TPS3JL | 6 | TPS3JL-6R | TGTTCCTAAATCATCTGCAAGCC | SEQ ID 576 |
| TPS3JL | 7 | TPS3JL-7F | GAATTGAATAGAGGCGACGTTCC | SEQ ID 577 |
| TPS3JL | 7 | TPS3JL-7R | ATAGTGCTGTTCTAGCCATATTTTTGC | SEQ ID 578 |
| TPS40JL | 1a | TPS40JL-1aF | CAATGTACTGTGGTCAATAATAGTAGCC | SEQ ID 579 |
| TPS40JL | 1a | TPS40JL-1aR | AATCGAAAGACCAAATCGGAGG | SEQ ID 580 |
| TPS40JL | 1b | TPS40JL-1bF | ACTGTGGTCAATAATAGTAGCCC | SEQ ID 581 |
| TPS40JL | 1b | TPS40JL-1bR | TGAATGTAATCGAAAGACCAAATCGG | SEQ ID 582 |
| TPS40JL | 2 | TPS40JL-2F | AATATAAGGGAGAATCCTATACAAGGC | SEQ ID 583 |
| TPS40JL | 2 | TPS40JL-2R | ATAGCCATGTTGGCGTAGAAGC | SEQ ID 584 |
| TPS40JL | 3a | TPS40JL-3aF | TGAGAAAAATGGTGAAAGCATATTGG | SEQ ID 585 |
| TPS40JL | 3a | TPS40JL-3aR | TCCGTTCTTGCAGTCCTCC | SEQ ID 586 |
| TPS40JL | 3b | TPS40JL-3bF | GAGGACTGCAAGAACGGAGG | SEQ ID 587 |
| TPS40JL | 3b | TPS40JL-3bR | ACAAATGTTTTAGATCGTCTTGATGC | SEQ ID 588 |
| TPS40JL | 4 | TPS40JL-4F | TTTGTGAGAGATAGGTTGATGGGG | SEQ ID 589 |
| TPS40JL | 4 | TPS40JL-4R | AAAGCTCTAATTCTTCTAATGTTCCG | SEQ ID 590 |
| TPS40JL | 6 | TPS40JL-6F | ATGGTTGGATTTCAGTAGGAGC | SEQ ID 591 |
| TPS40JL | 6 | TPS40JL-6R | GTACGATCATGGCAGATTGACG | SEQ ID 592 |
| TPS40JL | 7 | TPS40JL-7F | ATGCAAGACCATAATGTATCTAAAGAGG | SEQ ID 593 |
| TPS40JL | 7 | TPS40JL-7R | TGCAAAGTTTTTGGCATCATCAACC | SEQ ID 594 |
| TPS41JL | 1 | TPS41JL-1F | TCAATGTACTGTAGTCGATAGTTCTAATCC | SEQ ID 595 |
| TPS41JL | 1 | TPS41JL-1R | GACCAAATGGATGGTTCATAGTTGC | SEQ ID 596 |
| TPS41JL | 2a | TPS41JL-2aF | TAGAAGTCGGGTGAAAGAGATCG | SEQ ID 597 |
| TPS41JL | 2a | TPS41JL-2aR | AGAAGTCTAAATTCAAGAGCAATGG | SEQ ID 598 |
| TPS41JL | 2b | TPS41JL-2bF | ATAGAAGTCGGGTGAAAGAGATCG | SEQ ID 599 |
| TPS41JL | 2b | TPS41JL-2bR | AAGCGATAAGATACTCCAAGTCTTTGC | SEQ ID 600 |
| TPS41JL | 3 | TPS41JL-3F | ACGAGACAGGAAAATTCAAGGC | SEQ ID 601 |
| TPS41JL | 3 | TPS41JL-3R | TCCTCCAATGAAGTGGGAGC | SEQ ID 602 |
| TPS41JL | 4 | TPS41JL-4F | GTGGAGGCATACTAAACTTGGG | SEQ ID 603 |
| TPS41JL | 4 | TPS41JL-4R | AAAAGCTCTAATTCATCCAATGTTCC | SEQ ID 604 |
| TPS41JL | 5 | TPS41JL-5F | ACCAGAATACATGAAGATGCCT | SEQ ID 605 |
| TPS41JL | 5 | TPS41JL-5R | TGTTGATGGAGATGTCTTTGTCTCT | SEQ ID 606 |
| TPS41JL | 6 | TPS41JL-6F | GTGGATAGATATGTGTAGAGGTTTTCT | SEQ ID 607 |
| TPS41JL | 6 | TPS41JL-6R | AAGAACTGGTGCTCCCACTG | SEQ ID 608 |
| TPS41JL | 7 | TPS41JL-7F | TGTTCGGATGAAATGAAAAGAGGC | SEQ ID 609 |
| TPS41JL | 7 | TPS41JL-7R | TGTGACGCTCTACCAAGATTTTTGC | SEQ ID 610 |
| TPS42JL | 1 | TPS42JL-1F | CAAATGTTCTGTGGTCCATAACCC | SEQ ID 611 |
| TPS42JL | 1 | TPS42JL-1R | GACCAAATGGGAGGTTCATAGTTTCC | SEQ ID 612 |
| TPS42JL | 2 | TPS42JL-2F | GAGAAAGATGTGAAAATGATGCTACTTGG | SEQ ID 613 |
| TPS42JL | 2 | TPS42JL-2R | TGAGGTACTACAAATCCATACTCCC | SEQ ID 614 |
| TPS42JL | 3 | TPS42JL-3F | GATGATGAAACAGGAGAGTTCAAGG | SEQ ID 615 |
| TPS42JL | 3 | TPS42JL-3R | GACATTTGGTTGTGAAAATTCTTGC | SEQ ID 616 |
| TPS42JL | 4 | TPS42JL-4F | TGGATCATGCTTTGGAAATGCC | SEQ ID 617 |
| TPS42JL | 4 | TPS42JL-4R | AGCTCTTCTTGATATGTTGATTGC | SEQ ID 618 |
| TPS42JL | 5a | TPS42JL-5aF | TGGTGGAAGCATTCTAAACTTGG | SEQ ID 619 |
| TPS42JL | 5a | TPS42JL-5aR | AGCATTAGCGAAGAGTTGTAGTTCC | SEQ ID 620 |
| TPS42JL | 5b | TPS42JL-5bF | AGTGTTTCATGTGGCAAGTTGG | SEQ ID 621 |
| TPS42JL | 5b | TPS42JL-5bR | AGCGAAGAGTTGTAGTTCCTCC | SEQ ID 622 |
| TPS42JL | 6 | TPS42JL-6F | GGGATTTGAAAGTAATAGATGAGTTACCG | SEQ ID 623 |
| TPS42JL | 6 | TPS42JL-6R | AGAAAGCCATCTTCATGTAATCCG | SEQ ID 624 |
| TPS42JL | 7 | TPS42JL-7F | AGTGGATACCAACCAACATTGC | SEQ ID 625 |
| TPS42JL | 7 | TPS42JL-7R | TGTTATGAGATTTGTAAAACAGAAATAAGC | SEQ ID 626 |
| TPS42JL | 8a | TPS42JL-8aF | GATGAAATGAAAAGAGGCGATGTTCC | SEQ ID 627 |
| TPS42JL | 8a | TPS42JL-8aR | TCAAATGCTTGATGTGCTCACG | SEQ ID 628 |
| TPS42JL | 8b | TPS42JL-8bF | TGGAAGGAGATGAATAATGAAAATGG | SEQ ID 629 |
| TPS42JL | 8b | TPS42JL-8bR | GATCTTTTGATAGAGTATTTTGAGAAGC | SEQ ID 630 |
| TPS43JL | 1 | TPS43JL-1F | TCACTTAGAACCACAAAAGACCC | SEQ ID 631 |
| TPS43JL | 1 | TPS43JL-1R | GCCAACAAATAAGCCATGTTGC | SEQ ID 632 |
| TPS43JL | 2a | TPS43JL-2aF | ACAGATTTAGAGGCAGAGATGGG | SEQ ID 633 |
| TPS43JL | 2a | TPS43JL-2aR | GTGGTGAAACTCTTGGCTTCC | SEQ ID 634 |
| TPS43JL | 2b | TPS43JL-2bF | AGCTTCACACCTTGGAATGG | SEQ ID 635 |
| TPS43JL | 2b | TPS43JL-2bR | ATCCAACTTAGCCAACTCAAGC | SEQ ID 636 |
| TPS43JL | 3 | TPS43JL-3F | TGCAAGAGATCGTGTGGTGG | SEQ ID 637 |
| TPS43JL | 3 | TPS43JL-3R | ACTGCATTTGTGAAAAGTTCAAGC | SEQ ID 638 |
| TPS43JL | 4 | TPS43JL-4F | TGGGACATAAGGGCAATAAGGG | SEQ ID 639 |
| TPS43JL | 4 | TPS43JL-4R | CGATCACTTCATTACCAAAATTAAGCA | SEQ ID 640 |
| TPS43JL | 5 | TPS43JL-5F | TCAGTTGGTGGTCATGCAGC | SEQ ID 641 |
| TPS43JL | 5 | TPS43JL-5R | TGAAGTTCCTAAATCATCACTAAGCC | SEQ ID 642 |
| TPS43JL | 6 | TPS43JL-6F | TCAGTGGAGTGCTACATGGC | SEQ ID 643 |
| TPS43JL | 6 | TPS43JL-6R | TCACCATTATTTTTGGCAGGC | SEQ ID 644 |
| TPS44JL | 1a | TPS44JL-1aF | ACAAAGAGAGTTATGTGAAGATTATTGAGC | SEQ ID 645 |
| TPS44JL | 1a | TPS44JL-1aR | TCAAGTGTCTCTAAAGGGTTTTCC | SEQ ID 646 |
| TPS44JL | 1b | TPS44JL-1bF | ACTTCATGGTCTTCATCCTTTGG | SEQ ID 647 |
| TPS44JL | 1b | TPS44JL-1bR | TTCCCTAAGCAACCGAAAGC | SEQ ID 648 |
| TPS44JL | 2 | TPS44JL-2F | GCCCTAATGCACCCTATTCG | SEQ ID 649 |
| TPS44JL | 2 | TPS44JL-2R | TAATTCTTTTTGATGTAGTTGTTGAAGC | SEQ ID 650 |
| TPS44JL | 3 | TPS44JL-3F | TGATGACCAAGTTGATTTCTTTGC | SEQ ID 651 |
| TPS44JL | 3 | TPS44JL-3R | TTTCTAACTGCTTCAGTAAAAGGC | SEQ ID 652 |
| TPS44JL | 4 | TPS44JL-4F | ACTAGAGTACATGAAAGTATGTTACAAGA | SEQ ID 653 |
| TPS44JL | 4 | TPS44JL-4R | ACATAGCTCGCACAGTATGGA | SEQ ID 654 |
| TPS44JL | 5 | TPS44JL-5F | AGGAAGCCCAATGGTTACACA | SEQ ID 655 |
| TPS44JL | 5 | TPS44JL-5R | AGTTCGTATAATCTTAGGTTGGGGT | SEQ ID 656 |
| TPS44JL | 6 | TPS44JL-6F | GTAGTGGCTTCCGCTGTGG | SEQ ID 657 |
| TPS44JL | 6 | TPS44JL-6R | TCGCCTTCTCTATACATCTCATGG | SEQ ID 658 |
| TPS45JL | 1 | TPS45JL-1F | TGGTCTTCATCCTTTGGAAAACCC | SEQ ID 659 |
| TPS45JL | 1 | TPS45JL-1R | TTCCCTAAGCAACCGAAAGCG | SEQ ID 660 |
| TPS45JL | 2 | TPS45JL-2F | GTGAAACATGCCCTAATGCACC | SEQ ID 661 |
| TPS45JL | 2 | TPS45JL-2R | ATCTAGTTTGGCAGTTAAAAGAAGC | SEQ ID 662 |
| TPS45JL | 3 | TPS45JL-3F | ACAGAATAGTGGAGTGTTACATTTGG | SEQ ID 663 |
| TPS45JL | 3 | TPS45JL-3R | ATAATAGTAAGCAAAGAAATCAACTTGG | SEQ ID 664 |
| TPS45JL | 5 | TPS45JL-5F | TTCAAAACTCCCAAAAAGCGAAA | SEQ ID 665 |
| TPS45JL | 5 | TPS45JL-5R | TGTCACGTCAGCATACACTCC | SEQ ID 666 |
| TPS45JL | 6a | TPS45JL-6aF | TCAAAAGATGTAGTGGCTTCTGC | SEQ ID 667 |
| TPS45JL | 6a | TPS45JL-6aR | ATTAAGAGTGGTCTAGGAATAGCG | SEQ ID 668 |
| TPS45JL | 6b | TPS45JL-6bF | CAATATGGTGTAACAGATGAAGAAGC | SEQ ID 669 |
| TPS45JL | 6b | TPS45JL-6bR | TGAGAGTATCAATCAAATTCTTGAGC | SEQ ID 670 |
| TPS46JL | 1 | TPS46JL-1F | CATCGAGACAAACTGCGAATACT | SEQ ID 671 |
| TPS46JL | 1 | TPS46JL-1R | ATATGTGATCTCCATTTTCTTCCATTG | SEQ ID 672 |
| TPS46JL | 2 | TPS46JL-2F | CATGGATGGCCCGATTGGA | SEQ ID 673 |
| TPS46JL | 2 | TPS46JL-2R | TCGAAAATGATTCTTTCCAAGCCAT | SEQ ID 674 |
| TPS46JL | 4 | TPS46JL-4F | GATTGGGGACTTAGTGAAATGGG | SEQ ID 675 |
| TPS46JL | 4 | TPS46JL-4R | GTAAAGTTTTCTAACTCGGTGATTAAGG | SEQ ID 676 |
| TPS46JL | 5 | TPS46JL-5F | GGTGGGATGGTGAAGGATTGA | SEQ ID 677 |
| TPS46JL | 5 | TPS46JL-5R | TGATGTCACTTGTTTCTTGTTGAT | SEQ ID 678 |
| TPS46JL | 6 | TPS46JL-6F | TCACACCTTGCTTCTTCCAGC | SEQ ID 679 |
| TPS46JL | 6 | TPS46JL-6R | TCATTCAACAAACGACAAATAATCATAACC | SEQ ID 680 |
| TPS46JL | 7 | TPS46JL-7F | ACTTGAAGAACAATCCCGAGG | SEQ ID 681 |
| TPS46JL | 7 | TPS46JL-7R | AACCCTAACATCATCATCGTCG | SEQ ID 682 |
| TPS47JL | 10a | TPS47JL-10aF | CAGACACTGAAATGCTTGACG | SEQ ID 683 |
| TPS47JL | 10a | TPS47JL-10aR | TAGCGGTTAGACATTTAGAGGG | SEQ ID 684 |
| TPS47JL | 10b | TPS47JL-10bF | CAACCCTCTAAATGTCTAACCGC | SEQ ID 685 |
| TPS47JL | 10b | TPS47JL-10bR | AAATTGAGTTTTGAAGGCATAGTAGG | SEQ ID 686 |
| TPS47JL | 1a | TPS47JL-1aF | ATCTTTGCCCCAAACTCAGG | SEQ ID 687 |
| TPS47JL | 1a | TPS47JL-1aR | GGTTAGGATGTGGTATCATGGC | SEQ ID 688 |
| TPS47JL | 1b | TPS47JL-1bF | TACCACATCCTAACCAACCTTCG | SEQ ID 689 |
| TPS47JL | 1b | TPS47JL-1bR | CAACAACACAAGCCAGAGTGG | SEQ ID 690 |
| TPS47JL | 2 | TPS47JL-2F | TATTCATTCATCTAATGCAAAAAGGC | SEQ ID 691 |
| TPS47JL | 2 | TPS47JL-2R | AATATGTCCGAAACGAAAACGG | SEQ ID 692 |
| TPS47JL | 3a | TPS47JL-3aF | TGTTATCATATCTTGAAGTGTTGCC | SEQ ID 693 |
| TPS47JL | 3a | TPS47JL-3aR | CTGTTGCCGATGGAGATTGG | SEQ ID 694 |
| TPS47JL | 3b | TPS47JL-3bF | CAATCTCCATCGGCAACAGC | SEQ ID 695 |
| TPS47JL | 3b | TPS47JL-3bR | TGTTATTATTGGAAAACTTGTGAACTAGG | SEQ ID 696 |
| TPS47JL | 4 | TPS47JL-4F | AGGGTTAGCTGAGCATTTCGC | SEQ ID 697 |
| TPS47JL | 4 | TPS47JL-4R | CTAGGAAAAACTTTGTAGCCATGC | SEQ ID 698 |
| TPS47JL | 5a | TPS47JL-5aF | ACTATGAATGCTTTTCGGTTACG | SEQ ID 699 |
| TPS47JL | 5a | TPS47JL-5aR | AAAATAGACTTTTGAAGTAGTTTTCTCG | SEQ ID 700 |
| TPS47JL | 5b | TPS47JL-5bF | TTGCTTTTCATGGTGAATTTGAGC | SEQ ID 701 |
| TPS47JL | 5b | TPS47JL-5bR | TTTGTTAAAAGGATTTGTATGTGGAGC | SEQ ID 702 |
| TPS47JL | 6 | TPS47JL-6F | TGGCTCGACTAGATCACTTGG | SEQ ID 703 |
| TPS47JL | 6 | TPS47JL-6R | GTGAAACCTCGACAACCTTTGG | SEQ ID 704 |
| TPS47JL | 7 | TPS47JL-7F | GGGGACTTAATGAAATGGGATTTGG | SEQ ID 705 |
| TPS47JL | 7 | TPS47JL-7R | TCATAAGGCAAAGAACAACAAGC | SEQ ID 706 |
| TPS47JL | 9 | TPS47JL-9F | CCATAGCAACTCACACCTTGC | SEQ ID 707 |
| TPS47JL | 9 | TPS47JL-9R | ACACTCTCACATTGAATTGGTCG | SEQ ID 708 |
| TPS48JL | 1 | TPS48JL-1F | CTTGCTTTTCATGGTGAATTTGAGC | SEQ ID 709 |
| TPS48JL | 1 | TPS48JL-1R | CAGTTTCTTAAAGGGATTTGTATGTTGAGC | SEQ ID 710 |
| TPS48JL | 2 | TPS48JL-2F | GATGGCTCGACTAGATCACC | SEQ ID 711 |
| TPS48JL | 2 | TPS48JL-2R | AGATGTCTTTCCCATCCATAAAGC | SEQ ID 712 |
| TPS48JL | 4 | TPS48JL-4F | AGAATGGGGACTTAGTGAAATGGG | SEQ ID 713 |
| TPS48JL | 4 | TPS48JL-4R | TGGAGAATCATAAGGCAAATAACAGC | SEQ ID 714 |
| TPS48JL | 5 | TPS48JL-5F | TGGAATAATGAAGGGTTGAGTGGT | SEQ ID 715 |
| TPS48JL | 5 | TPS48JL-5R | TCTTATGTAGCTGGTTATGTCTTCA | SEQ ID 716 |
| TPS48JL | 6a | TPS48JL-6aF | TGAATCTGAGTGGAGTAAAAGTGG | SEQ ID 717 |
| TPS48JL | 6a | TPS48JL-6aR | AGAAGAAATGAAGCTGGAAGAAGC | SEQ ID 718 |
| TPS48JL | 6b | TPS48JL-6bF | GCAACTCATACCTTGCTTCTTCC | SEQ ID 719 |
| TPS48JL | 6b | TPS48JL-6bR | GAACTTTGTAAGTCATTCAACAAACG | SEQ ID 720 |
| TPS48JL | 7a | TPS48JL-7aF | GAGGGAAAGAGAAGAAGGCAAACC | SEQ ID 721 |
| TPS48JL | 7a | TPS48JL-7aR | ACTTTTAAGCAAGATAAGTGTAGAAGCC | SEQ ID 722 |
| TPS48JL | 7b | TPS48JL-7bF | AATGACAATGGTTTTACCAATCTTCC | SEQ ID 723 |
| TPS48JL | 7b | TPS48JL-7bR | AATCGCTTTGTTAATGTCTTCAAGC | SEQ ID 724 |
| TPS49JL | 1 | TPS49JL-1F | TCGGCCAATTTTCATCCTAGT | SEQ ID 725 |
| TPS49JL | 1 | TPS49JL-1R | GGTATATTTGAGAAAGTGATCTCCCC | SEQ ID 726 |
| TPS49JL | 2 | TPS49JL-2F | GGATGCTAACTGCTGCCCC | SEQ ID 727 |
| TPS49JL | 2 | TPS49JL-2R | AAAGGGAGACCGTATGAAGGG | SEQ ID 728 |
| TPS49JL | 3a | TPS49JL-3aF | GAAATCCCAGTTGACCATGAGC | SEQ ID 729 |
| TPS49JL | 3a | TPS49JL-3aR | GATGACTGTGGTTAGGGTCG | SEQ ID 730 |
| TPS49JL | 3b | TPS49JL-3bF | CACAACTCATTTGGTGGAGGC | SEQ ID 731 |
| TPS49JL | 3b | TPS49JL-3bR | AGATGACTGTGGTTAGGGTCG | SEQ ID 732 |
| TPS49JL | 4a | TPS49JL-4aF | GGAAAGACTTGGACTTTGCATCG | SEQ ID 733 |
| TPS49JL | 4a | TPS49JL-4aR | CTAGCAGCACTGTACTTTGGC | SEQ ID 734 |
| TPS49JL | 4b | TPS49JL-4bF | GGAAAGACTTGGACTTTGCATCG | SEQ ID 735 |
| TPS49JL | 4b | TPS49JL-4bR | TTGCGTCTGTGAAGAGTTCC | SEQ ID 736 |
| TPS49JL | 5 | TPS49JL-5F | TATACGATGAAATTGAGGAGGAGTTGGC | SEQ ID 737 |
| TPS49JL | 5 | TPS49JL-5R | AGGCCATCCGATAAGTTCTGCC | SEQ ID 738 |
| TPS49JL | 6a | TPS49JL-6aF | TACTACGTGGAAGCTCAATGG | SEQ ID 739 |
| TPS49JL | 6a | TPS49JL-6aR | AAGCCAATGAAAGACCTCAGC | SEQ ID 740 |
| TPS49JL | 6b | TPS49JL-6bF | ACTACATGCTCACTGCCACG | SEQ ID 741 |
| TPS49JL | 6b | TPS49JL-6bR | TCTGCAAACTACTGCTGATGC | SEQ ID 742 |
| TPS49JL | 7a | TPS49JL-7aF | GTTTGAGCAAGATAGAGGACACG | SEQ ID 743 |
| TPS49JL | 7a | TPS49JL-7aR | GCATGAGGACAGACATGGGC | SEQ ID 744 |
| TPS49JL | 7b | TPS49JL-7bF | TGAATGAGGAGTGCATGGAGC | SEQ ID 745 |
| TPS49JL | 7b | TPS49JL-7bR | TCCGGCATGGGTGTAACCG | SEQ ID 746 |
| TPS4FN | 1 | TPS4FN-1F | AGCCTCATCACAAAATGACAAAGT | SEQ ID 747 |
| TPS4FN | 1 | TPS4FN-1R | GGTTGATAAGTTGTTGTTGGACGA | SEQ ID 748 |
| TPS4FN | 2 | TPS4FN-2F | AAGGAAGTGGTAAGGAGAGAGG | SEQ ID 749 |
| TPS4FN | 2 | TPS4FN-2R | GAAACCATAAATCCATGTTGGCG | SEQ ID 750 |
| TPS4FN | 3a | TPS4FN-3aF | AGCAAGGGAATTTTAAGGAGTGC | SEQ ID 751 |
| TPS4FN | 3a | TPS4FN-3aR | AGGTGAGTGGTGGTGAAAGC | SEQ ID 752 |
| TPS4FN | 3b | TPS4FN-3bF | TTGCTTTCACCACCACTCACC | SEQ ID 753 |
| TPS4FN | 3b | TPS4FN-3bR | GAAATGTAATGCCTGGCGTGG | SEQ ID 754 |
| TPS4FN | 4a | TPS4FN-4aF | TGCAAGAGATAGGATTGTGGAGC | SEQ ID 755 |
| TPS4FN | 4a | TPS4FN-4aR | TCTGCAACTGAGGCCAATGC | SEQ ID 756 |
| TPS4FN | 4b | TPS4FN-4bF | TGAACCTGAATTGTCACTGGC | SEQ ID 757 |
| TPS4FN | 4b | TPS4FN-4bR | GCTCAAGCTCTTCAAATGTACC | SEQ ID 758 |
| TPS4FN | 5 | TPS4FN-5F | AATTGTGCGGATCAACTTCG | SEQ ID 759 |
| TPS4FN | 5 | TPS4FN-5R | TGTAACTTTCCTCCTTTCCAAGC | SEQ ID 760 |
| TPS4FN | 6 | TPS4FN-6F | AGCGATGAAAAGATTACTTGGAGC | SEQ ID 761 |
| TPS4FN | 6 | TPS4FN-6R | CATCCATGAACCTACAAAGGATATTGC | SEQ ID 762 |
| TPS4FN | 7a | TPS4FN-7aF | AGAGATCATTCACCGTCTACCG | SEQ ID 763 |
| TPS4FN | 7a | TPS4FN-7aR | AAACTGGATAAGGCACATTAGAAGGC | SEQ ID 764 |
| TPS4FN | 7b | TPS4FN-7bF | GGCCTTCTAATGTGCCTTATCC | SEQ ID 765 |
| TPS4FN | 7b | TPS4FN-7bR | TCATGGGATTTGATCTATAAGTAACGC | SEQ ID 766 |
| TPS4JL | 1a | TPS4JL-1aF | AGTTTTAGCCTCATCACAAAATGACA | SEQ ID 767 |
| TPS4JL | 1a | TPS4JL-1aR | GTTGATAAGTTGTTGTTGGACGAA | SEQ ID 768 |
| TPS4JL | 1b | TPS4JL-lbF | TAGCCTCATCACAAAATGACAAAG | SEQ ID 769 |
| TPS4JL | 1b | TPS4JL-lbR | CCCCCAAATAGAAGGTTGATAAGT | SEQ ID 770 |
| TPS4JL | 2a | TPS4JL-2aF | TAAAAAGCAGCGAGTTGACG | SEQ ID 771 |
| TPS4JL | 2a | TPS4JL-2aR | AATGATACGACAATCCCAAACG | SEQ ID 772 |
| TPS4JL | 2b | TPS4JL-2bF | AAGGAAGTGGTAAGGAGAGAGG | SEQ ID 773 |
| TPS4JL | 2b | TPS4JL-2bR | GAAACCATAAATCCATGTTGGCG | SEQ ID 774 |
| TPS4JL | 3a | TPS4JL-3aF | AGCAAGGGAATTTTAAGGAGTGC | SEQ ID 775 |
| TPS4JL | 3a | TPS4JL-3aR | AGGTGAGTGGTGGTGAAAGC | SEQ ID 776 |
| TPS4JL | 3b | TPS4JL-3bF | CTTGCTTTCACCACCACTCACC | SEQ ID 777 |
| TPS4JL | 3b | TPS4JL-3bR | GTAATGCCTGGCGTGGAGC | SEQ ID 778 |
| TPS4JL | 4a | TPS4JL-4aF | GCAAGAGATAGGATTGTGGAGC | SEQ ID 779 |
| TPS4JL | 4a | TPS4JL-4aR | ATCTGCAACTGAGGCCAAGG | SEQ ID 780 |
| TPS4JL | 4b | TPS4JL-4bF | TGAACCTGAATTGTCACTGGC | SEQ ID 781 |
| TPS4JL | 4b | TPS4JL-4bR | GCTCAAGCTCTTCAAATGTACC | SEQ ID 782 |
| TPS4JL | 5 | TPS4JL-5F | AATTGTGCGGATCAACTTCG | SEQ ID 783 |
| TPS4JL | 5 | TPS4JL-5R | TGTAACTTTCCTCCTTTCCAAGC | SEQ ID 784 |
| TPS4JL | 6 | TPS4JL-6F | CAGTGAAGCTCGATGGTTGC | SEQ ID 785 |
| TPS4JL | 6 | TPS4JL-6R | TCCATGAACCTACAAAGGATATTGC | SEQ ID 786 |
| TPS4JL | 7a | TPS4JL-7aF | CCGTTGAGAGTTACATGAGGC | SEQ ID 787 |
| TPS4JL | 7a | TPS4JL-7aR | ACTGGATAAGGCACATTAGAAGG | SEQ ID 788 |
| TPS4JL | 7b | TPS4JL-7bF | ACAAGAGGCATGTGATGAGC | SEQ ID 789 |
| TPS4JL | 7b | TPS4JL-7bR | GGGATTTGATCTATAAGTAACGCAGC | SEQ ID 790 |
| TPS4-likeJL | 1 | TPS4-likeJL-1F | TGTCGTCTCAAATCTTAGCAACC | SEQ ID 791 |
| TPS4-likeJL | 1 | TPS4-likeJL-1R | ATGCAAAAATCGGTCTCCCC | SEQ ID 792 |
| TPS4-likeJL | 2a | TPS4-likeJL-2aF | TTCTTATGTGATGATTGGAGTAATCG | SEQ ID 793 |
| TPS4-likeJL | 2a | TPS4-likeJL-2aR | GCTTATTTTGTTGTAAATGTGTTGAAGC | SEQ ID 794 |
| TPS4-IikeJL | 2b | TPS4-IikeJL-2bF | CGATTGAAGTTAATTGATGTGGTGC | SEQ ID 795 |
| TPS4-likeJL | 2b | TPS4-likeJL-2bR | AAAGAAACCCTATATCCATGTTGTCG | SEQ ID 796 |
| TPS4-likeJL | 3a | TPS4-likeJL-3aF | GAATGTTTGGCGAGTGACACC | SEQ ID 797 |
| TPS4-likeJL | 3a | TPS4-likeJL-3aR | AAGGGCCTCTCTAGGGCTCG | SEQ ID 798 |
| TPS4-likeJL | 3b | TPS4-likeJL-3bF | AAGAACACCCCAATGATGATCC | SEQ ID 799 |
| TPS4-likeJL | 3b | TPS4-likeJL-3bR | ACTAAGCTCCTTTTTGTGCATGG | SEQ ID 800 |
| TPS4-likeJL | 4 | TPS4-likeJL-4F | GGTGGAAGGAATTAGACAGTGC | SEQ ID 801 |
| TPS4-likeJL | 4 | TPS4-likeJL-4R | TCAGCGATTGAGGAAAGTGC | SEQ ID 802 |
| TPS4-likeJL | 5 | TPS4-likeJL-5F | GGGACAAAAATTGTATGGATAAACTCC | SEQ ID 803 |
| TPS4-likeJL | 5 | TPS4-likeJL-5R | TCCTTTTCAAACTCTTGTTCAAATTCC | SEQ ID 804 |
| TPS4-likeJL | 6a | TPS4-likeJL-6aF | AGCTCGATGGTTGAATGAAGG | SEQ ID 805 |
| TPS4-likeJL | 6a | TPS4-likeJL-6aR | GGTCTTTGGAGAGCCACTCG | SEQ ID 806 |
| TPS4-likeJL | 6b | TPS4-likeJL-6bF | TGATGGCTTGCTCTTTAGTTGG | SEQ ID 807 |
| TPS4-likeJL | 6b | TPS4-likeJL-6bR | AGCCACGTCATCCATGTACC | SEQ ID 808 |
| TPS4-likeJL | 7a | TPS4-likeJL-7aF | AGAATGAGCAAGAGAGAAATCATATACC | SEQ ID 809 |
| TPS4-likeJL | 7a | TPS4-likeJL-7aR | CCATGCAATAACCACTCGCC | SEQ ID 810 |
| TPS4-likeJL | 7b | TPS4-likeJL-7bF | TCTCAAACCAACTGAAGCAGC | SEQ ID 811 |
| TPS4-likeJL | 7b | TPS4-likeJL-7bR | TTGGATCAATGAGCAAGACAGC | SEQ ID 812 |
| TPS50JL | 1 | TPS50JL-1F | GGTCTTCATCCTTTGGAAAATCC | SEQ ID 813 |
| TPS50JL | 1 | TPS50JL-1R | AAGCAACCGAAATCGAAGAGC | SEQ ID 814 |
| TPS50JL | 2 | TPS50JL-2F | AGCTTCACAAATGAGAGTTCGC | SEQ ID 815 |
| TPS50JL | 2 | TPS50JL-2R | TCGTGAGAAGGTAGTTGATGG | SEQ ID 816 |
| TPS50JL | 3 | TPS50JL-3F | ACAGAATAGTGGAGTGTTACATTTGG | SEQ ID 817 |
| TPS50JL | 3 | TPS50JL-3R | ATAATAGTAAGCAAAGAAATCAACTTGG | SEQ ID 818 |
| TPS50JL | 4 | TPS50JL-4F | ACAACTTGCCAGAGTACATGAAAG | SEQ ID 819 |
| TPS50JL | 4 | TPS50JL-4R | CGCGCAGTATGAATTTTCCTTTG | SEQ ID 820 |
| TPS50JL | 5 | TPS50JL-5F | GGATCTGAAGAGCTAATATCAATGGC | SEQ ID 821 |
| TPS50JL | 5 | TPS50JL-5R | AGTTCGTATAATCTTAGGTTGGGG | SEQ ID 822 |
| TPS50JL | 6a | TPS50JL-6aF | TCAAAAGATGTAGTGGCTTCTGC | SEQ ID 823 |
| TPS50JL | 6a | TPS50JL-6aR | ATTAAGAGTGGTCTAGGAATAGCG | SEQ ID 824 |
| TPS50JL | 6b | TPS50JL-6bF | CAATATGGTGTAACAGATGAAGAAGC | SEQ ID 825 |
| TPS50JL | 6b | TPS50JL-6bR | TGAGAGTATCAATCAAATTCTTGAGC | SEQ ID 826 |
| TPS51JL | 1 | TPS51JL-1F | AGCCAACTTTGAACCATCCA | SEQ ID 827 |
| TPS51JL | 1 | TPS51JL-1R | AAAGAGACTGAATGAAATCAAAAGACCA | SEQ ID 828 |
| TPS51JL | 2 | TPS51JL-2F | GAGGAAGATGTGAAAAGGATGC | SEQ ID 829 |
| TPS51JL | 2 | TPS51JL-2R | GCCTAAATTCAAGAGCAGTGGC | SEQ ID 830 |
| TPS51JL | 3a | TPS51JL-3aF | CAGGAAAATTCAAAACAAACATAAGTGG | SEQ ID 831 |
| TPS51JL | 3a | TPS51JL-3aR | AGCTTCCTCTAAAATGCTTTCGC | SEQ ID 832 |
| TPS51JL | 3b | TPS51JL-3bF | AGGCGAAAGCATTTTAGAGGAAGC | SEQ ID 833 |
| TPS51JL | 3b | TPS51JL-3bR | AGCTTCTATTCTTGTGGTCCTTCG | SEQ ID 834 |
| TPS51JL | 4 | TPS51JL-4F | GGTGGAGGCATACTAAACTTGG | SEQ ID 835 |
| TPS51JL | 4 | TPS51JL-4R | TGAAAAGCTCTAATTCATCTAATGTTCC | SEQ ID 836 |
| TPS51JL | 5 | TPS51JL-5F | GATGGGATGTGGAAATGATAAATGA | SEQ ID 837 |
| TPS51JL | 5 | TPS51JL-5R | ATTTTGATGGTGATGTGTTGATCT | SEQ ID 838 |
| TPS51JL | 6a | TPS51JL-6aF | CTACAAGAAGCAAAATGGTATTACAGTGG | SEQ ID 839 |
| TPS51JL | 6a | TPS51JL-6aR | GCACAATAAGAACTGGTGCTCCC | SEQ ID 840 |
| TPS51JL | 6b | TPS51JL-6bF | GGAGCACCAGTTCTTATTGTGC | SEQ ID 841 |
| TPS51JL | 6b | TPS51JL-6bR | GCACTGTGACGAATTATGGTAGG | SEQ ID 842 |
| TPS51JL | 7 | TPS51JL-7F | AAAGAGGTGATGCTCCGACG | SEQ ID 843 |
| TPS51JL | 7 | TPS51JL-7R | AGATTATCCTGAGAACTGTGACC | SEQ ID 844 |
| TPS52JL | 1 | TPS52JL-1F | TCAGAAGAGATCAGCAAACTATCAAC | SEQ ID 845 |
| TPS52JL | 1 | TPS52JL-1R | CTTGAAAGGAGTAGAAAGTGATTGA | SEQ ID 846 |
| TPS52JL | 2 | TPS52JL-2F | TGCAAAGACTTGGAATCTCTTACC | SEQ ID 847 |
| TPS52JL | 2 | TPS52JL-2R | CATACACATTTTTGTTGGTGTTGC | SEQ ID 848 |
| TPS52JL | 3 | TPS52JL-3F | GAGGCAATTTTATGGTGTCTTCC | SEQ ID 849 |
| TPS52JL | 3 | TPS52JL-3R | TTGTTTTGTGTCTTGTCTCTTCC | SEQ ID 850 |
| TPS52JL | 4 | TPS52JL-4F | GGACAATAGGAGTTTCATTTGAGCC | SEQ ID 851 |
| TPS52JL | 4 | TPS52JL-4R | TCTCAACCACATTAGTGAAGAGC | SEQ ID 852 |
| TPS52JL | 5 | TPS52JL-5F | TGGGATGTTAGTGCTATGAATGGG | SEQ ID 853 |
| TPS52JL | 5 | TPS52JL-5R | GAGGCTTTTTCCTTTTAACACATCAAATGC | SEQ ID 854 |
| TPS52JL | 6 | TPS52JL-6F | TAAGAGAAGCAAGATGGTATTATGATGG | SEQ ID 855 |
| TPS52JL | 6 | TPS52JL-6R | GTATTATGGTAGGGTATCCATTAAAGC | SEQ ID 856 |
| TPS52JL | 7 | TPS52JL-7F | CGTGTATGTGAAGAGAAAGCTCG | SEQ ID 857 |
| TPS52JL | 7 | TPS52JL-7R | AGTACATGATCTTTTGTTTGGCG | SEQ ID 858 |
| TPS53JL | 1 | TPS53JL-1F | CAGCCAACTTTGAACCATCCA | SEQ ID 859 |
| TPS53JL | 1 | TPS53JL-1R | GCTTGAAAGAGACTGAACGAAA | SEQ ID 860 |
| TPS53JL | 2 | TPS53JL-2F | CGACGAAGATGTGAAAAGGATGC | SEQ ID 861 |
| TPS53JL | 2 | TPS53JL-2R | ACCATGTTGACGTAGAAGCC | SEQ ID 862 |
| TPS53JL | 3a | TPS53JL-3aF | AGCTAGAATTTTCACAACTGAACG | SEQ ID 863 |
| TPS53JL | 3a | TPS53JL-3aR | GCTTCTATTCTTGTGGTCCTTCG | SEQ ID 864 |
| TPS53JL | 3b | TPS53JL-3bF | TACGATATTGAAGTAGTGAATCATGC | SEQ ID 865 |
| TPS53JL | 3b | TPS53JL-3bR | TTGGCAAACTCAAGCAAAATAGG | SEQ ID 866 |
| TPS53JL | 4 | TPS53JL-4F | TGGTGGAGGCATACTAAACTTGG | SEQ ID 867 |
| TPS53JL | 4 | TPS53JL-4R | GTGAAAAGCTCTAATTCATCTAATGTTCC | SEQ ID 868 |
| TPS53JL | 5 | TPS53JL-5F | GATGGGATGTGGAAATGATAAATGAA | SEQ ID 869 |
| TPS53JL | 5 | TPS53JL-5R | ATTTTGATGGTGATTTGTTGATCTCT | SEQ ID 870 |
| TPS53JL | 6a | TPS53JL-6aF | CTACAAGAAGCAAAATGGTACTACAGTGG | SEQ ID 871 |
| TPS53JL | 6a | TPS53JL-6aR | GCACAATAAGAACTGGTGCTCCC | SEQ ID 872 |
| TPS53JL | 6b | TPS53JL-6bF | GGAGCACCAGTTCTTATTGTGC | SEQ ID 873 |
| TPS53JL | 6b | TPS53JL-6bR | GCACTGTGACGAATTATGGTAGG | SEQ ID 874 |
| TPS53JL | 7 | TPS53JL-7F | AAGAGGTGATGCTCCGACG | SEQ ID 875 |
| TPS53JL | 7 | TPS53JL-7R | TAGATTATCCTGAGAACTGTGACC | SEQ ID 876 |
| TPS54JL | 1a | TPS54JL-1aF | ACAAAGAGAGTTATGTGAAGATTATTGAGC | SEQ ID 877 |
| TPS54JL | 1a | TPS54JL-1aR | CCGAAAGCGAAGAGCATCAGC | SEQ ID 878 |
| TPS54JL | 1b | TPS54JL-1bF | AATTACTTCATGGTCTTCAACCTTTGG | SEQ ID 879 |
| TPS54JL | 1b | TPS54JL-1bR | CTATAAAATAGCCTTGTTCCCTAAGC | SEQ ID 880 |
| TPS54JL | 2a | TPS54JL-2aF | AGCTTCACAAATGAGAGTTCGC | SEQ ID 881 |
| TPS54JL | 2a | TPS54JL-2aR | CGGATAGGGTGCATTAGGGC | SEQ ID 882 |
| TPS54JL | 2b | TPS54JL-2bF | ATGCACCCTATCCGAAAGAGC | SEQ ID 883 |
| TPS54JL | 2b | TPS54JL-2bR | TTCCTTTTGATGTAGTTGTTGAAGC | SEQ ID 884 |
| TPS54JL | 3 | TPS54JL-3F | ACAGAATAGTGGAGTGTTACATTTGG | SEQ ID 885 |
| TPS54JL | 3 | TPS54JL-3R | ATAATAGTAAGCAAAGAAATCAACTTGG | SEQ ID 886 |
| TPS54JL | 4 | TPS54JL-4F | TCATCAGGTGGGATATTTTTGCT | SEQ ID 887 |
| TPS54JL | 4 | TPS54JL-4R | ACATAACTCGCGCAGAATGAA | SEQ ID 888 |
| TPS54JL | 5 | TPS54JL-5F | GGATCTGAAGAGCTAATATCAATGGC | SEQ ID 889 |
| TPS54JL | 5 | TPS54JL-5R | TCGTATAATGTTAGGTTGGGGC | SEQ ID 890 |
| TPS54JL | 6 | TPS54JL-6F | TCAAAAGATGTAGTGGCTTCTGC | SEQ ID 891 |
| TPS54JL | 6 | TPS54JL-6R | ATTAAGAGTGGTCTAGGAATAGCG | SEQ ID 892 |
| TPS55JL | 1 | TPS55JL-1F | ACAAAAGGTTGAGGAATTAAAAGAGG | SEQ ID 893 |
| TPS55JL | 1 | TPS55JL-1R | CCCACAAGGAAACATCATGC | SEQ ID 894 |
| TPS55JL | 2 | TPS55JL-2F | CACACCTAAATGAGTTTTTGGCG | SEQ ID 895 |
| TPS55JL | 2 | TPS55JL-2R | CCTGCTGATTTCACTAAGCTCC | SEQ ID 896 |
| TPS55JL | 3 | TPS55JL-3F | TGTGGATGTTAGGAGTCTATTATGAACC | SEQ ID 897 |
| TPS55JL | 3 | TPS55JL-3R | ATTTCTTGCCAAAGAGTATTGGGG | SEQ ID 898 |
| TPS55JL | 4 | TPS55JL-4F | TTGTTATGAAGAGTTTGAGCAAGTG | SEQ ID 899 |
| TPS55JL | 4 | TPS55JL-4R | CTCTATAAGTTTCTTCTTTTGTAAGCAC | SEQ ID 900 |
| TPS55JL | 5 | TPS55JL-5F | GAAGCTCGATGGTTGAATAGTGG | SEQ ID 901 |
| TPS55JL | 5 | TPS55JL-5R | CATGAGCCTACAAATAATAACAGATGC | SEQ ID 902 |
| TPS55JL | 6 | TPS55JL-6F | AAATTAGCTTCACCCATATTACTTAGG | SEQ ID 903 |
| TPS55JL | 6 | TPS55JL-6R | TGGGATCAATTAGCAAAGCAGC | SEQ ID 904 |
| TPS56JL | 1 | TPS56JL-1F | TCCACTCAAATCTTAGCATCATCA | SEQ ID 905 |
| TPS56JL | 1 | TPS56JL-1R | TGGATGAAATGTTTTTGTAGGACGA | SEQ ID 906 |
| TPS56JL | 2 | TPS56JL-2F | GAAAAGGTTGAGGAATTAAAAGAAGTGG | SEQ ID 907 |
| TPS56JL | 2 | TPS56JL-2R | GTTTCAAAATGATAAGACAATGCCAAACG | SEQ ID 908 |
| TPS56JL | 3 | TPS56JL-3F | AAAATGTTTGGCAAGTGATACCC | SEQ ID 909 |
| TPS56JL | 3 | TPS56JL-3R | GGGCCTCTCTAGGGCTCG | SEQ ID 910 |
| TPS56JL | 4 | TPS56JL-4F | TAGGATTGTGGAATTGTACCTTTGG | SEQ ID 911 |
| TPS56JL | 4 | TPS56JL-4R | TGCATCATAATCAGTAATTGAGGC | SEQ ID 912 |
| TPS56JL | 5a | TPS56JL-5aF | AGGACGTTTTGAATTGTTATGAAGAGT | SEQ. ID 913 |
| TPS56JL | 5a | TPS56JL-5aR | TCTTTATGTTACTTCTTTTTCTAGCACT | SEQ. ID 914 |
| TPS56JL | 5b | TPS56JL-5bF | ACTCAATCAAGAATACATGCAAACAT | SEQ. ID 915 |
| TPS56JL | 5b | TPS56JL-5bR | GCACTTGCTCAAACTCTTCATAAC | SEQ. ID 916 |
| TPS56JL | 6a | TPS56JL-6aF | TGTTTGCATAGAGGACTCATCCC | SEQ ID 917 |
| TPS56JL | 6a | TPS56JL-6aR | TCTTTCGATCTTTGGAGAGCC | SEQ ID 918 |
| TPS56JL | 6b | TPS56JL-6bF | GTTTGCATAGAGGACTCATCCC | SEQ ID 919 |
| TPS56JL | 6b | TPS56JL-6bR | AGTTTTCATTCCAACCAAAGAGC | SEQ ID 920 |
| TPS56JL | 7a | TPS56JL-7aF | TGGTGTTTGTGAAGAAGAAGCC | SEQ ID 921 |
| TPS56JL | 7a | TPS56JL-7aR | GGTGAAGCTACTTGAGTTGGC | SEQ ID 922 |
| TPS56JL | 7b | TPS56JL-7bF | TTTGTGAAGCCAACTCAAGTAGC | SEQ ID 923 |
| TPS56JL | 7b | TPS56JL-7bR | ATTGGATGAATAAGCAAAGCAGC | SEQ ID 924 |
| TPS57JL | 1 | TPS57JL-1F | TCATCCTTTGGAAAACCCTTTGG | SEQ ID 925 |
| TPS57JL | 1 | TPS57JL-1R | AAGCAACCGAAAGCAAAGAGC | SEQ ID 926 |
| TPS57JL | 2 | TPS57JL-2F | CATATTCGACAAGTACAAGAATGAAAAAGG | SEQ ID 927 |
| TPS57JL | 2 | TPS57JL-2R | CTCCATGAACTCTCATTTGTGC | SEQ ID 928 |
| TPS57JL | 3 | TPS57JL-3F | GGAGTGCTATTTCTGGGTTTATGG | SEQ ID 929 |
| TPS57JL | 3 | TPS57JL-3R | AGGCTCTAGTTCTTCAAGTGTACC | SEQ ID 930 |
| TPS57JL | 5 | TPS57JL-5F | GTTGGGGTTGATAGTGCAGG | SEQ ID 931 |
| TPS57JL | 5 | TPS57JL-5R | TCATTCATAACTCTTCCAACTATTGCC | SEQ ID 932 |
| TPS57JL | 6 | TPS57JL-6F | GGAGCGGAAGAAATCATCAGG | SEQ ID 933 |
| TPS57JL | 6 | TPS57JL-6R | TGGGTTGTGTAAAGCCATCTCC | SEQ ID 934 |
| TPS58JL | 1 | TPS58JL-1F | ATTCAACGATTGGGGTTGTCTT | SEQ ID 935 |
| TPS58JL | 1 | TPS58JL-1R | CCGAAAGCAAAGAGCATCAGC | SEQ ID 936 |
| TPS58JL | 2 | TPS58JL-2F | CATATTCGACAAGTACAAGAATGAAAAAGG | SEQ ID 937 |
| TPS58JL | 2 | TPS58JL-2R | CTCCATGAACTCTCATTTGTGC | SEQ ID 938 |
| TPS58JL | 3a | TPS58JL-3aF | AGAATAGTGGAGTGCTATTTCTGG | SEQ ID 939 |
| TPS58JL | 3a | TPS58JL-3aR | ATTATTAGTCTGATTTGGGAAGTTTCTGC | SEQ ID 940 |
| TPS58JL | 3b | TPS58JL-3bF | TGGAGTGCTATTTCTGGGTTTATGG | SEQ ID 941 |
| TPS58JL | 3b | TPS58JL-3bR | ATTTGGTGATTATTAGTCTGATTTGGG | SEQ ID 942 |
| TPS58JL | 5 | TPS58JL-5F | GTTGGGGTTGATAGTGCAGG | SEQ ID 943 |
| TPS58JL | 5 | TPS58JL-5R | TCATTCATAACTCTTCCAACTATTGCC | SEQ ID 944 |
| TPS58JL | 6a | TPS58JL-6aF | GAAATTGTGGCTTCAACTGTGG | SEQ ID 945 |
| TPS58JL | 6a | TPS58JL-6aR | GAGCAAAGTGGGTTGTGTAAAGC | SEQ ID 946 |
| TPS58JL | 6b | TPS58JL-6bF | AGAAATCATCAGGAGAAATTGTGGC | SEQ ID 947 |
| TPS58JL | 6b | TPS58JL-6bR | TGGGTTGTGTAAAGCCATCTCC | SEQ ID 948 |
| TPS59JL | 1 | TPS59JL-1F | CACACAAATCTTAGTATCTTCAAATGACA | SEQ ID 949 |
| TPS59JL | 1 | TPS59JL-1R | CAAATCTTGTTGTGAAATGTTGTAATG | SEQ ID 950 |
| TPS59JL | 2 | TPS59JL-2F | AAAAGGTTGAGGAATTAAAAGAAGTGG | SEQ ID 951 |
| TPS59JL | 2 | TPS59JL-2R | AGTTTCAAAATGATAAGACAATCCCAAACG | SEQ ID 952 |
| TPS59JL | 3a | TPS59JL-3aF | TGAAAAATTCAAAGACGAGGATGGG | SEQ ID 953 |
| TPS59JL | 3a | TPS59JL-3aR | CATTTAGGTGTGTTGTTGTGAAAGC | SEQ ID 954 |
| TPS59JL | 3b | TPS59JL-3bF | ACTTGCTTTCACAACAACACACC | SEQ ID 955 |
| TPS59JL | 3b | TPS59JL-3bR | AAGGGCCTCTCTAGGGCTCG | SEQ ID 956 |
| TPS59JL | 3c | TPS59JL-3cF | ACACCATGAAGACGATGATCC | SEQ ID 957 |
| TPS59JL | 3c | TPS59JL-3cR | ACCTGCTGACTTCACTAAGTTCC | SEQ ID 958 |
| TPS59JL | 4 | TPS59JL-4F | AGATAGGATTGTGGAATTATACCTTTGG | SEQ ID 959 |
| TPS59JL | 4 | TPS59JL-4R | AGCTCAAGTTCTTCAAGTGTACC | SEQ ID 960 |
| TPS59JL | 5 | TPS59JL-5F | TTTGAATTGTTATGAAGAGTTTGAGC | SEQ ID 961 |
| TPS59JL | 5 | TPS59JL-5R | TGTATTGTTCTTCTTTTTCTAGCACTT | SEQ ID 962 |
| TPS59JL | 6 | TPS59JL-6F | ATTTGGATGAAGCTCGATGTTTGC | SEQ ID 963 |
| TPS59JL | 6 | TPS59JL-6R | CCATGAGCCTACAAATAGTAACAGATGC | SEQ ID 964 |
| TPS59JL | 7a | TPS59JL-7aF | TGAGGAACATTCAGCAGTGG | SEQ ID 965 |
| TPS59JL | 7a | TPS59JL-7aR | GCTTCAAAAACTCTTCATTTATTTCTTTCC | SEQ ID 966 |
| TPS59JL | 7b | TPS59JL-7bF | AAGAAATAAATGAAGAGTTTTTGAAGCC | SEQ ID 967 |
| TPS59JL | 7b | TPS59JL-7bR | CCAACGTGTGTATAACCATCTCC | SEQ ID 968 |
| TPS5FN | 1 | TPS5FN-1F | ATGTCACTATCAGGACTAATCTCC | SEQ ID 969 |
| TPS5FN | 1 | TPS5FN-1R | AAAATGAGCATCCCACAATGG | SEQ ID 970 |
| TPS5FN | 2 | TPS5FN-2F | AGCTATGGGAAAAGAATCAATGAGC | SEQ ID 971 |
| TPS5FN | 2 | TPS5FN-2R | GAGTTTGAAATGAAGAGCAGTGG | SEQ ID 972 |
| TPS5FN | 3 | TPS5FN-3F | GACGAGAAAAAGGAGTTCAAGG | SEQ ID 973 |
| TPS5FN | 3 | TPS5FN-3R | TCATCAAGTATTGTTTGAGATGTTTGG | SEQ ID 974 |
| TPS5FN | 4 | TPS5FN-4F | GCCGAAAAATGCTCACAAAGATTGG | SEQ ID 975 |
| TPS5FN | 4 | TPS5FN-4R | CTTCCCAATGCGTGTTGAAAGAGG | SEQ ID 976 |
| TPS5FN | 5 | TPS5FN-5F | ACCAACTTCCAGATTACATGAAGATA | SEQ ID 977 |
| TPS5FN | 5 | TPS5FN-5R | TAGCACGTCATACGCCATTT | SEQ ID 978 |
| TPS5FN | 6 | TPS5FN-6F | GAACCCTTAATTCTAGTCAATCTTTATTGC | SEQ ID 979 |
| TPS5FN | 6 | TPS5FN-6R | TTCCTAAATCATCAACAAGTCGAGC | SEQ ID 980 |
| TPS5FN | 7a | TPS5FN-7aF | TGAACTGAAAAGAGGAGACAATCC | SEQ ID 981 |
| TPS5FN | 7a | TPS5FN-7aR | AATGGAGACTCACCCACTCG | SEQ ID 982 |
| TPS5FN | 7b | TPS5FN-7bF | GCCATAGATTTCGTTAGGACAGC | SEQ ID 983 |
| TPS5FN | 7b | TPS5FN-7bR | GGGAATGGAAGTGAAGAACAAGG | SEQ ID 984 |
| TPS5JL | 1 | TPS5JL-1F | TCAAAGAACAACCAGCAATAGTCC | SEQ ID 985 |
| TPS5JL | 1 | TPS5JL-1R | TTGGAGTGATTGGATAAAATGAGC | SEQ ID 986 |
| TPS5JL | 2a | TPS5JL-2aF | GCTATGGGAAAAGAATCAATGAGC | SEQ ID 987 |
| TPS5JL | 2a | TPS5JL-2aR | AGTTTGAAATGAAGAGCAGTGG | SEQ ID 988 |
| TPS5JL | 2b | TPS5JL-2bF | TCTTGAGAAGGAGGCTGAAAATCC | SEQ ID 989 |
| TPS5JL | 2b | TPS5JL-2bR | GGAGTTTGAAATGAAGAGCAGTGG | SEQ ID 990 |
| TPS5JL | 3a | TPS5JL-3aF | GGAGAGTTTGAGTAAAGATGTGAAAGG | SEQ ID 991 |
| TPS5JL | 3a | TPS5JL-3aR | TCCAACCTTTTCATTCTCCAATGC | SEQ ID 992 |
| TPS5JL | 3b | TPS5JL-3bF | TTTGAGTAAAGATGTGAAAGGAATGG | SEQ ID 993 |
| TPS5JL | 3b | TPS5JL-3bR | GATGTTTGGTTGTGAAATCTCTTGC | SEQ ID 994 |
| TPS5JL | 4 | TPS5JL-4F | GAGCCACAGTTTAGATATTGCCG | SEQ ID 995 |
| TPS5JL | 4 | TPS5JL-4R | AGAGGCTTAGCTCATCAAGCG | SEQ ID 996 |
| TPS5JL | 5 | TPS5JL-5F | CGACCAACTTCCAGATTACATGAAG | SEQ ID 997 |
| TPS5JL | 5 | TPS5JL-5R | AGCACGTCATACGCCATTTCA | SEQ ID 998 |
| TPS5JL | 6a | TPS5JL-6aF | TGGAGGCAAATTGGTATCATAGTGG | SEQ ID 999 |
| TPS5JL | 6a | TPS5JL-6aR | AAAGGTGGGATATTGAAGCAAGC | SEQ ID 1000 |
| TPS5JL | 6b | TPS5JL-6bF | GAACCCTTAATTCTAGTCAATCTTTATTGC | SEQ ID 1001 |
| TPS5JL | 6b | TPS5JL-6bR | TTCCTAAATCATCAACAAGTCGAGC | SEQ ID 1002 |
| TPS5JL | 7a | TPS5JL-7aF | TGAACTGAAAAGAGGAGACAATCC | SEQ ID 1003 |
| TPS5JL | 7a | TPS5JL-7aR | AATGGAGACTCACCCACTCG | SEQ ID 1004 |
| TPS5JL | 7b | TPS5JL-7bF | TGGAAGGAAATGAATGAAGCTCG | SEQ ID 1005 |
| TPS5JL | 7b | TPS5JL-7bR | ACCCCATCTTGCTCCTTTTGG | SEQ ID 1006 |
| TPS60JL | 1 | TPS60JL-1F | GAGCTTGACTATGTTGAAAACCC | SEQ ID 1007 |
| TPS60JL | 1 | TPS60JL-1R | ATAACCTTGTTGTCTAACCAATCG | SEQ ID 1008 |
| TPS60JL | 2a | TPS60JL-2aF | GCTTCACAACTAAGAGTGCATGG | SEQ ID 1009 |
| TPS60JL | 2a | TPS60JL-2aR | TAATGTCTAGCCTGTCTCCTTTGC | SEQ ID 1010 |
| TPS60JL | 2b | TPS60JL-2bF | AACGATATTCGAGGAATGTTAAGC | SEQ ID 1011 |
| TPS60JL | 2b | TPS60JL-2bR | AGGTTTTAGCTCCAGATTCGAGG | SEQ ID 1012 |
| TPS60JL | 3 | TPS60JL-3F | TAATGGATTGTTACTTTTGGACTTTTGG | SEQ ID 1013 |
| TPS60JL | 3 | TPS60JL-3R | GTATTGCTTCAGTTAAGAGGTGTAGC | SEQ ID 1014 |
| TPS60JL | 5 | TPS60JL-5F | TCAACTACCAGAGTACATTCAACC | SEQ ID 1015 |
| TPS60JL | 5 | TPS60JL-5R | GCAATATGATTTGTCCTTAGTAAATTCTCC | SEQ ID 1016 |
| TPS60JL | 6 | TPS60JL-6F | CACTTGTTACTGCTGCTTCTCC | SEQ ID 1017 |
| TPS60JL | 6 | TPS60JL-6R | GCCAATGTCGTTAAGAACTCTACC | SEQ ID 1018 |
| TPS60JL | 7 | TPS60JL-7F | TCTGAATGAAGAGTGTCTCTATCCC | SEQ ID 1019 |
| TPS60JL | 7 | TPS60JL-7R | AGCTTCTCTTGTAAAACCATCTCC | SEQ ID 1020 |
| TPS61JL | 1a | TPS61JL-1aF | ATCATCGAAATTGAAAGACACAGG | SEQ ID 1021 |
| TPS61JL | 1a | TPS61JL-1aR | GGGCAAGATAGTTGCTCTGC | SEQ ID 1022 |
| TPS61JL | 1b | TPS61JL-1bF | GAGCAACTATCTTGCCCAACC | SEQ ID 1023 |
| TPS61JL | 1b | TPS61JL-lbR | TCATATTTGTTGTTAAGAGACTCCAGG | SEQ ID 1024 |
| TPS61JL | 2 | TPS61JL-2F | AGCTTATCGAGGATGTGAGGC | SEQ ID 1025 |
| TPS61JL | 2 | TPS61JL-2R | TCCATGAAGCCTAAGAAGCC | SEQ ID 1026 |
| TPS61JL | 3 | TPS61JL-3F | TGTTTCACAAGGCATATTTGTTGG | SEQ ID 1027 |
| TPS61JL | 3 | TPS61JL-3R | AGTTCCAAAGCATGAACCACC | SEQ ID 1028 |
| TPS61JL | 4 | TPS61JL-4F | GTTGGTGGAAGAATGTGGGC | SEQ ID 1029 |
| TPS61JL | 4 | TPS61JL-4R | GGCATTGGTGAAGTGTCTGAGC | SEQ ID 1030 |
| TPS61JL | 5 | TP561JL-5F | TGTACGGGAAACTGAAAAACTTCC | SEQ ID 1031 |
| TP561JL | 5 | TP561JL-5R | CCCTTTAGATGAGGTAAAACTAACTTGC | SEQ ID 1032 |
| TP561JL | 6 | TP561JL-6F | GCTTGGATTTCATCTTCGGGC | SEQ ID 1033 |
| TP561JL | 6 | TP561JL-6R | CGCTGAAGTTCCTAAATCGTTGC | SEQ ID 1034 |
| TP561JL | 7 | TP561JL-7F | CCTCTCATTCCCAATAAGTAAACTAGC | SEQ ID 1035 |
| TP561JL | 7 | TP561JL-7R | TCCTCAAAGTACAGGAGACATCG | SEQ ID 1036 |
| TPS62JL | 1 | TPS62JL-1F | TTGATGCTATTCAACGGCTAGG | SEQ ID 1037 |
| TPS62JL | 1 | TPS62JL-1R | CAGCAGGAACGAAGTGACCG | SEQ ID 1038 |
| TPS62JL | 2 | TPS62JL-2F | ATATGAAGCCTCCCATCTATGC | SEQ ID 1039 |
| TPS62JL | 2 | TPS62JL-2R | TGGGATAGACACCAAAAAGGTCC | SEQ ID 1040 |
| TPS62JL | 3 | TPS62JL-3F | GGTGGCGAGACATTGGTTTAGC | SEQ ID 1041 |
| TPS62JL | 3 | TPS62JL-3R | ATGGATTTTGTAAGCGCAACCC | SEQ ID 1042 |
| TPS62JL | 4 | TPS62JL-4F | TATAGAAAAACTTCCAGACTCCATGA | SEQ ID 1043 |
| TPS62JL | 4 | TPS62JL-4R | TAAAGGGCTCCATCCACGC | SEQ ID 1044 |
| TPS62JL | 5 | TPS62JL-5F | GGGCAAGTTTGTGCGAAGC | SEQ ID 1045 |
| TPS62JL | 5 | TPS62JL-5R | TGGCACTACCAAAGTCATCCC | SEQ ID 1046 |
| TPS62JL | 6a | TPS62JL-6aF | AGGACATGACGGATCTTATGTGG | SEQ ID 1047 |
| TPS62JL | 6a | TPS62JL-6aR | AGGAATGGTGGTGGAAACGC | SEQ ID 1048 |
| TPS62JL | 6b | TPS62JL-6bF | TCCACCACCATTCCTCAAAGC | SEQ ID 1049 |
| TPS62JL | 6b | TPS62JL-6bR | ATGTTCTTCCAAGTGGGGTAGG | SEQ ID 1050 |
| TPS63JL | 1 | TPS63JL-1F | GAACTTGGTCAACGCTGTGC | SEQ ID 1051 |
| TPS63JL | 1 | TPS63JL-1R | ACCTCCTTGTCTCAGTAATCGG | SEQ ID 1052 |
| TPS63JL | 2a | TPS63JL-2aF | TAGTGGAAGAAGACAGAGAGGG | SEQ ID 1053 |
| TPS63JL | 2a | TPS63JL-2aR | AGTGTTGGCTGAGAAACTGTCC | SEQ ID 1054 |
| TPS63JL | 2b | TPS63JL-2bF | TCTCAGCCAACACTGCTACC | SEQ ID 1055 |
| TPS63JL | 2b | TPS63JL-2bR | GAGACTTGAACAATTTCCCTTTGG | SEQ ID 1056 |
| TPS63JL | 3 | TPS63JL-3F | GGTGGAAAGAGCTTGGTTTGG | SEQ ID 1057 |
| TPS63JL | 3 | TPS63JL-3R | GAGTGAGTTCGTCTAGTGTCC | SEQ ID 1058 |
| TPS63JL | 4 | TPS63JL-4F | GGGAAATTAAAGAGCAGTTACCCG | SEQ ID 1059 |
| TPS63JL | 4 | TPS63JL-4R | GGGTTCCATCCATGTTTTCTGT | SEQ ID 1060 |
| TPS63JL | 5a | TPS63JL-5aF | GGTTTGGTTGTGGGAAGTTGC | SEQ ID 1061 |
| TPS63JL | 5a | TPS63JL-5aR | CTGCTGTAGAAGACACAAGGC | SEQ ID 1062 |
| TPS63JL | 5b | TPS63JL-5bF | GAACGCGATTGTGAGTTCTGG | SEQ ID 1063 |
| TPS63JL | 5b | TPS63JL-5bR | GGCACTTCCTAAGTCATCCC | SEQ ID 1064 |
| TPS63JL | 6a | TPS63JL-6aF | ATGGGCATGATGGGTCTTACG | SEQ ID 1065 |
| TPS63JL | 6a | TPS63JL-6aR | CTCCTTATTGAGGCGTTCCC | SEQ ID 1066 |
| TPS63JL | 6b | TPS63JL-6bF | GCAAGGGAGCAAGTGATTCG | SEQ ID 1067 |
| TPS63JL | 6b | TPS63JL-6bR | CTGGAGGCTTGGAAGGCG | SEQ ID 1068 |
| TPS64JL | 1 | TPS64JL-1F | ACTCGGCTACTTTCTCAAAGAGG | SEQ ID 1069 |
| TPS64JL | 1 | TPS64JL-1R | ATGGATTACCAAAACGAGGAAGC | SEQ ID 1070 |
| TPS64JL | 2a | TPS64JL-2aF | AGGCTCATAGATAGCATCCAACGG | SEQ ID 1071 |
| TPS64JL | 2a | TPS64JL-2aR | GTAGCCGAAAACGAAGCCCC | SEQ ID 1072 |
| TPS64JL | 2b | TPS64JL-2bF | TCCTTCAGATGTTGTCAGATTTCAATTCC | SEQ ID 1073 |
| TPS64JL | 2b | TPS64JL-2bR | GAGGTAGTTGGAAAGCCATTATGC | SEQ ID 1074 |
| TPS64JL | 3a | TPS64JL-3aF | ATGCTGAGCTTGTATGAGGC | SEQ ID 1075 |
| TPS64JL | 3a | TPS64JL-3aR | CAAGAAGAGCAGGGCAGTGG | SEQ ID 1076 |
| TPS64JL | 3b | TPS64JL-3bF | GACACCTGAGGATGGCACC | SEQ ID 1077 |
| TPS64JL | 3b | TPS64JL-3bR | TTGTGGAGTGACTGAAGCTCG | SEQ ID 1078 |
| TPS64JL | 4 | TPS64JL-4F | GTGGAAACAGTTGGGTCTGG | SEQ ID 1079 |
| TPS64JL | 4 | TPS64JL-4R | GTGGAGTTCATCCAAAGATCCG | SEQ ID 1080 |
| TPS64JL | 5 | TPS64JL-5F | GATGGGATCTTGGTGCAATGG | SEQ ID 1081 |
| TPS64JL | 5 | TPS64JL-5R | ACATATTTTCATGTACTCAGGAAGC | SEQ ID 1082 |
| TPS64JL | 6 | TPS64JL-6F | TGAAATTGGCTACAGAGTTCTCA | SEQ ID 1083 |
| TPS64JL | 6 | TPS64JL-6R | AGTGTTGTGTAACGCATAATCCA | SEQ ID 1084 |
| TPS64JL | 7a | TPS64JL-7aF | GCATTTCTAACCGAAGCAGAATGG | SEQ ID 1085 |
| TPS64JL | 7a | TPS64JL-7aR | TGGCTGTTCCCAAATCATCCC | SEQ ID 1086 |
| TPS64JL | 7b | TPS64JL-7bF | CACTCATTTTTCCTCATAGGTCATGG | SEQ ID 1087 |
| TPS64JL | 7b | TPS64JL-7bR | TTGGCTGTTCCCAAATCATCCC | SEQ ID 1088 |
| TPS64JL | 8a | TPS64JL-8aF | AGAGAGGAGATGTTGCTTCTAGC | SEQ ID 1089 |
| TPS64JL | 8a | TPS64JL-8aR | AAGGTCAAAACAGGCCGTGG | SEQ ID 1090 |
| TPS64JL | 8b | TPS64JL-8bF | AGCTCGTGGATAGAGCTAAACG | SEQ ID 1091 |
| TPS64JL | 8b | TPS64JL-8bR | CCATGTTGATAGATGACTTGGGC | SEQ ID 1092 |
| TPS6FN | 1 | TPS6FN-1F | ATGCTACCCCATCCAATGTGC | SEQ ID 1093 |
| TPS6FN | 1 | TPS6FN-1R | AATATAATCGAAAGACCAAATGGAGGGC | SEQ ID 1094 |
| TPS6FN | 2 | TPS6FN-2F | TTAGTTGAGATGGAAAACTCTTTAGC | SEQ ID 1095 |
| TPS6FN | 2 | TPS6FN-2R | ATAGCCATGTTGACGTAGAAGC | SEQ ID 1096 |
| TPS6FN | 3 | TPS6FN-3F | TCCCACTTCATCGGAGGACC | SEQ ID 1097 |
| TPS6FN | 3 | TPS6FN-3R | TTGGCTAACTCAAGCAACATAGG | SEQ ID 1098 |
| TPS6FN | 4 | TPS6FN-4F | GGTGGAGACATACTAAACTTGGAGA | SEQ ID 1099 |
| TPS6FN | 4 | TPS6FN-4R | GCTTTGGTGAAAAGCTCTAATTCAT | SEQ ID 1100 |
| TPS6FN | 5 | TPS6FN-5F | ATGAGTTACCAGAATACATGAAGATGC | SEQ ID 1101 |
| TPS6FN | 5 | TPS6FN-5R | GGTATTGAATGTTGATGGAGATTTCTTGG | SEQ ID 1102 |
| TPS6FN | 6a | TPS6FN-6aF | GGTCGATATGTGTAAAAGTTTCTTGC | SEQ ID 1103 |
| TPS6FN | 6a | TPS6FN-6aR | ACTGGTGCTCCTACTGAAATCC | SEQ ID 1104 |
| TPS6FN | 6b | TPS6FN-6bF | AATGGTTGGATTTCAGTAGGAGC | SEQ ID 1105 |
| TPS6FN | 6b | TPS6FN-6bR | TGGCACTGTGACGAATAATGG | SEQ. ID 1106 |
| TPS6FN | 7a | TPS6FN-7aF | ATTGAAAAGAGGTGATGCTCCG | SEQ. ID 1107 |
| TPS6FN | 7a | TPS6FN-7aR | TTGGATTGATTATCTTGAGAACTATGACC | SEQ ID 1108 |
| TPS6FN | 7b | TPS6FN-7bF | ATTGTATCTGAAGAGGAAGCTCG | SEQ ID 1109 |
| TPS6FN | 7b | TPS6FN-7bR | TATAAGGGAATAGGTTCAATAATCAAGG | SEQ ID 1110 |
| TPS6JL | 1 | TPS6JL-1F | ATGTGCTGTGGTCAATAGTTCT | SEQ ID 1111 |
| TPS6JL | 1 | TPS6JL-1R | AAAGACCAAATGGAGGGCTCA | SEQ ID 1112 |
| TPS6JL | 2 | TPS6JL-2F | ACAGGTCGAGTCAAAGAATTGG | SEQ ID 1113 |
| TPS6JL | 2 | TPS6JL-2R | GCCATGTTGACGTAGAAGCC | SEQ ID 1114 |
| TPS6JL | 3a | TPS6JL-3aF | CTCCCACTTCATTGGAGGACT | SEQ ID 1115 |
| TPS6JL | 3a | TPS6JL-3aR | AGCAAACTCAAGCAAAATAGGATT | SEQ ID 1116 |
| TPS6JL | 3b | TPS6JL-3bF | GCTAAGTGGTTCATCGACGC | SEQ ID 1117 |
| TPS6JL | 3b | TPS6JL-3bR | TGATGTGTTGATTGTATCATGTTGA | SEQ ID 1118 |
| TPS6JL | 4 | TPS6JL-4F | GGTGGAGGCATACTAAACTTGG | SEQ ID 1119 |
| TPS6JL | 4 | TPS6JL-4R | GAAAAGCTCTAATTCATCCAATGTTCC | SEQ ID 1120 |
| TPS6JL | 5 | TPS6JL-5F | GATGGGATATGGAAATGATAAATGAGT | SEQ ID 1121 |
| TPS6JL | 5 | TPS6JL-5R | TGTTGATGGAGATGTGTTGGTCT | SEQ ID 1122 |
| TPS6JL | 6 | TPS6JL-6F | GATTTCAGTGGGAGCACCG | SEQ ID 1123 |
| TPS6JL | 6 | TPS6JL-6R | ACTATGACGAATTATGGCGGG | SEQ ID 1124 |
| TPS6JL | 7 | TPS6JL-7F | GAACTGAAAAGAGGTGATGCTCC | SEQ ID 1125 |
| TPS6JL | 7 | TPS6JL-7R | TTTGGATTGATTATCTTGAGAACTATGACC | SEQ ID 1126 |
| TPS6-likeJL | 1 | TPS6-likeJL-1F | ATGCTACCCCATCCAATGTGC | SEQ ID 1127 |
| TPS6-likeJL | 1 | TPS6-likeJL-1R | AATATAATCAAAAGACCAAATGGAGGGC | SEQ ID 1128 |
| TPS6-likeJL | 2 | TPS6-likeJL-2F | CAACTTGAACTCATTGATACATTGC | SEQ ID 1129 |
| TPS6-likeJL | 2 | TPS6-likeJL-2R | TAGCCATGTTGACGTAGAAGCC | SEQ ID 1130 |
| TPS6-likeJL | 3 | TPS6-likeJL-3F | AGCTCCCACTTCATCGGAGG | SEQ ID 1131 |
| TPS6-likeJL | 3 | TPS6-likeJL-3R | TTTGGCTAACTCAAGCAACATAGG | SEQ ID 1132 |
| TPS6-likeJL | 4 | TPS6-likeJL-4F | GGTGGAGACATACTAAACTTGGAGA | SEQ ID 1133 |
| TPS6-likeJL | 4 | TPS6-likeJL-4R | GCTTTGGTGAAAAGCTCTAATTCAT | SEQ ID 1134 |
| TPS6-likeJL | 5 | TPS6-likeJL-5F | AGTTACCAGAATACATGAAGATGCC | SEQ ID 1135 |
| TPS6-likeJL | 5 | TPS6-likeJL-5R | TTCTTGGTCTCTTAACACCTCAAA | SEQ ID 1136 |
| TPS6-likeJL | 6a | TPS6-likeJL-6aF | GGTCGATATGTGTAAAAGTTTCTTGC | SEQ ID 1137 |
| TPS6-likeJL | 6a | TPS6-likeJL-6aR | ACTGGTGCTCCTACTGAAATCC | SEQ ID 1138 |
| TPS6-likeJL | 6b | TPS6-likeJL-6bF | GAAAATGGTTGGATTTCAGTAGGAGC | SEQ ID 1139 |
| TPS6-likeJL | 6b | TPS6-likeJL-6bR | TGGCACTGTGACGAATAATGGC | SEQ ID 1140 |
| TPS6-likeJL | 7 | TPS6-likeJL-7F | AATTGAAAAGAGGTGATGCTCCG | SEQ ID 1141 |
| TPS6-likeJL | 7 | TPS6-likeJL-7R | TTTGGATTGATTATCTTGAGAACTATGACC | SEQ ID 1142 |
| TPS7FN | 1 | TPS7FN-1F | AGTCAAGTGTTAGCTTCATCTCA | SEQ ID 1143 |
| TPS7FN | 1 | TPS7FN-1R | CGCCCCAAATAGAAGGGTGA | SEQ ID 1144 |
| TPS7FN | 2 | TPS7FN-2F | GAGAGTGAAATTGAGAAATTGTTGG | SEQ ID 1145 |
| TPS7FN | 2 | TPS7FN-2R | TGTCTTAATAATCTAAACCAAAGAGAAGC | SEQ ID 1146 |
| TPS7FN | 3a | TPS7FN-3aF | TGATAACCGACGTTTCGGG | SEQ ID 1147 |
| TPS7FN | 3a | TPS7FN-3aR | CTAGCATGAAGCCTCTCTAAGG | SEQ ID 1148 |
| TPS7FN | 3b | TPS7FN-3bF | GGAAAGGCCACTAAGAATGACC | SEQ ID 1149 |
| TPS7FN | 3b | TPS7FN-3bR | ACCTCACAATTTCACTAAGCTCC | SEQ ID 1150 |
| TPS7FN | 4 | TPS7FN-4F | GTGGAAGGAGCATGAGTTTGC | SEQ ID 1151 |
| TPS7FN | 4 | TPS7FN-4R | TGACTTTGGTTAGAAGTTTTCTTGC | SEQ ID 1152 |
| TPS7FN | 6a | TPS7FN-6aF | GAAGAAGCTCGATGGTTAAATGAAGG | SEQ ID 1153 |
| TPS7FN | 6a | TPS7FN-6aR | AGTAGAAGCTGAAACAATCTTAGGG | SEQ ID 1154 |
| TPS7FN | 6b | TPS7FN-6bF | TCTGGTTACGTTTTGTTGATAGC | SEQ ID 1155 |
| TPS7FN | 6b | TPS7FN-6bR | TGAATCTTGAGAGGAGAGTAGAAGC | SEQ ID 1156 |
| TPS7FN | 7 | TPS7FN-7F | TGAAGCAATATGAGGTTTCAGAGG | SEQ ID 1157 |
| TPS7FN | 7 | TPS7FN-7R | ATGGGATGGGATCTATAAGTAAAGC | SEQ ID 1158 |
| TPS8FN | 1 | TPS8FN-1F | AAGTCTTAGCTTCATCTCAATTATGTGAC | SEQ ID 1159 |
| TPS8FN | 1 | TPS8FN-1R | TCGATCACCCCAAATAGAAGGG | SEQ ID 1160 |
| TPS8FN | 2 | TPS8FN-2F | CGAGAGTGAAATCGAGAAATTATTGG | SEQ ID 1161 |
| TPS8FN | 2 | TPS8FN-2R | CACTTTGTCTTAATAGTCTGAACCG | SEQ ID 1162 |
| TPS8FN | 3 | TPS8FN-3F | AGCTTGTATGAGGCTTCGC | SEQ ID 1163 |
| TPS8FN | 3 | TPS8FN-3R | TCTCTATGGTCTTTCTTAATGGCG | SEQ ID 1164 |
| TPS8FN | 4 | TPS8FN-4F | GGTGGAACTGTATTTTTGGATATTGGG | SEQ ID 1165 |
| TPS8FN | 4 | TPS8FN-4R | ACCTTTGAATTGCTTTGGTAAGAAGC | SEQ ID 1166 |
| TPS8FN | 6a | TPS8FN-6aF | GAAGCTCGATGGTTGAATGAAGG | SEQ ID 1167 |
| TPS8FN | 6a | TPS8FN-6aR | GCGAGAGCCTATGTCATCC | SEQ ID 1168 |
| TPS8FN | 6b | TPS8FN-6bF | TGTGGTTACGTTATGTTGATAGCC | SEQ ID 1169 |
| TPS8FN | 6b | TPS8FN-6bR | AATCTTGATAGGAGAGTGGAAGC | SEQ ID 1170 |
| TPS8FN | 7 | TPS8FN-7F | GTTTGAGCAAGAGAGAAATCACATACC | SEQ ID 1171 |
| TPS8FN | 7 | TPS8FN-7R | TGTAAAATTAAGAACACGAACTAAGATAGG | SEQ ID 1172 |
| TPS8JL | 1 | TPS8JL-1F | TCAAGTCTTAGCTTCATCTCAATTATGT | SEQ ID 1173 |
| TPS8JL | 1 | TPS8JL-1R | TCGATCACCCCAAATAGAAGG | SEQ ID 1174 |
| TPS8JL | 2 | TPS8JL-2F | TTTGAGAGTGAAATTGAGAAATTGTTGG | SEQ ID 1175 |
| TPS8JL | 2 | TPS8JL-2R | TAAATCCATGTTGTCTTAATAATCTAAACC | SEQ ID 1176 |
| TPS8JL | 3a | TPS8JL-3aF | GACGCTGAAGGTAATTTTAAGAAAAGC | SEQ ID 1177 |
| TPS8JL | 3a | TPS8JL-3aR | AAGGTGAGTGGTTGTGAAAGC | SEQ ID 1178 |
| TPS8JL | 3b | TPS8JL-3bF | GCTTCACACTTGAGTTATGTTGG | SEQ ID 1179 |
| TPS8JL | 3b | TPS8JL-3bR | CTCTCTATGGTCTTTCTTAGAGGC | SEQ ID 1180 |
| TPS8JL | 4 | TPS8JL-4F | AGGATGGTGGAACTGTATTTTTGG | SEQ ID 1181 |
| TPS8JL | 4 | TPS8JL-4R | CCTTTGAATTGCTTTGGTAAGAAGC | SEQ ID 1182 |
| TPS8JL | 6 | TPS8JL-6F | GAAGAAGCTCGATGGTTGAATGAAGG | SEQ ID 1183 |
| TPS8JL | 6 | TPS8JL-6R | AGAGTGGAAGCTGAAACAATCTTAGG | SEQ ID 1184 |
| TPS8JL | 7a | TPS8JL-7aF | TGAGCAACAGAGAAATCACATACC | SEQ ID 1185 |
| TPS8JL | 7a | TPS8JL-7aR | GAACACGAAGTAAGATAGGAAAAGGC | SEQ ID 1186 |
| TPS8JL | 7b | TPS8JL-7bF | GTGCCTTTTCCTATCTTACTTCG | SEQ ID 1187 |
| TPS8JL | 7b | TPS8JL-7bR | CAATGCTTTCTTTGAGCACTTTTCC | SEQ ID 1188 |
| TPS8-likeJL | 2 | TPS8-likeJL-2F | TTTGAGAGTGAAATTGAGAAATTGTTGG | SEQ ID 1189 |
| TPS8-likeJL | 2 | TPS8-likeJL-2R | TAAATCCATGTTGTCTTAATAATCTAAACC | SEQ ID 1190 |
| TPS8-likeJL | 3a | TPS8-likeJL-3aF | GCTTGATAACCGATGTTTCGGG | SEQ ID 1191 |
| TPS8-likeJL | 3a | TPS8-likeJL-3aR | GAGGTGAGTGGTTGTGAAAGC | SEQ ID 1192 |
| TPS8-likeJL | 3b | TPS8-likeJL-3bF | TCACCTCAAGGCTATTGTGGC | SEQ ID 1193 |
| TPS8-likeJL | 3b | TPS8-likeJL-3bR | AGATGTAAAACCTAGCATGAAGCC | SEQ ID 1194 |
| TPS8-likeJL | 4 | TPS8-likeJL-4F | GGTGGAAGGAGCATGAGTTTGC | SEQ ID 1195 |
| TPS8-likeJL | 4 | TPS8-likeJL-4R | TTGAGGCTAATGCAATGACTTTGG | SEQ ID 1196 |
| TPS8-likeJL | 5 | TPS8-likeJL-5F | GGGACATAAATTGTCTGGATAAACTTGA | SEQ ID 1197 |
| TPS8-likeJL | 5 | TPS8-IikeJL-5R | TTTTTAAGCTCCTTTTCAAATTCTTCATAA | SEQ ID 1198 |
| TPS8-likeJL | 6 | TPS8-likeJL-6F | AGCTCGATGGTTGAGTGAAGG | SEQ ID 1199 |
| TPS8-likeJL | 6 | TPS8-likeJL-6R | ACCTAGCAAGTAGAGTGGAAGC | SEQ ID 1200 |
| TPS8-likeJL | 7a | TPS8-likeJL-7aF | TGAGCAAAAGAGAAATCACATACC | SEQ ID 1201 |
| TPS8-likeJL | 7a | TPS8-likeJL-7aR | TTTCTTTCCAGTGGGTGTCC | SEQ ID 1202 |
| TPS8-likeJL | 7b | TPS8-likeJL-7bF | TATGGGGTATCAGAGAAAGAGGC | SEQ ID 1203 |
| TPS8-likeJL | 7b | TPS8-likeJL-7bR | GAACACGAACTAAGATAGGAAAAGGC | SEQ ID 1204 |
| TPS8-likeJL | 7c | TPS8-likeJL-7cF | GTGCCTTTTCCTATCTTAGTTCG | SEQ ID 1205 |
| TPS8-likeJL | 7c | TPS8-IikeJL-7cR | CAATGCTTTCTTTGAGCACTTTTCC | SEQ ID 1206 |
| TPS9FN | 1 | TPS9FN-1F | TCCACTCAAATCTTAGCAACCTC | SEQ ID 1207 |
| TPS9FN | 1 | TPS9FN-1R | TGCAAAAATCGGTCTCCCCA | SEQ ID 1208 |
| TPS9FN | 2a | TPS9FN-2aF | TGAAGTTAATTGATGTGGTAGAACG | SEQ ID 1209 |
| TPS9FN | 2a | TPS9FN-2aR | AGAAACCCTATATCCATGTTGTCG | SEQ ID 1210 |
| TPS9FN | 2b | TPS9FN-2bF | CAAGTTGAAGAATTGAAAGAAGTGG | SEQ ID 1211 |
| TPS9FN | 2b | TPS9FN-2bR | AAATGATAGGACAATCCCAAACG | SEQ ID 1212 |
| TPS9FN | 3a | TPS9FN-3aF | GCGAGTGACACCGTTGGTT | SEQ ID 1213 |
| TPS9FN | 3a | TPS9FN-3aR | TGGATCATCATTGGGGTGTTCTTT | SEQ ID 1214 |
| TPS9FN | 3b | TPS9FN-3bF | AAAGAACACCCCAATGATGATCC | SEQ ID 1215 |
| TPS9FN | 3b | TPS9FN-3bR | GGGTCTTTCTTAAGGGCCTC | SEQ ID 1216 |
| TPS9FN | 4 | TPS9FN-4F | TGGTGGAAGGAATTAGACAGTGC | SEQ ID 1217 |
| TPS9FN | 4 | TPS9FN-4R | ATCAGCGATTGAGGAAAGTGC | SEQ ID 1218 |
| TPS9FN | 5 | TPS9FN-5F | GATAAACTCCATCCAGAATACTTGC | SEQ ID 1219 |
| TPS9FN | 5 | TPS9FN-5R | ACTTTGTAAGTTTCCTCCTTTTCAA | SEQ ID 1220 |
| TPS9FN | 6a | TPS9FN-6aF | GAAGCTCGATGGTTGAATGAAGG | SEQ ID 1221 |
| TPS9FN | 6a | TPS9FN-6aR | CGCTTGCCCTAACAATCTTGG | SEQ ID 1222 |
| TPS9FN | 6b | TPS9FN-6bF | GTTGATGGCTTGCTCTTTAGTTGG | SEQ ID 1223 |
| TPS9FN | 6b | TPS9FN-6bR | TGTGACCAGCCACGTCATCC | SEQ ID 1224 |
| TPS9FN | 7 | TPS9FN-7F | TGTGATGAAATGAATAGGCGAGTGG | SEQ ID 1225 |
| TPS9FN | 7 | TPS9FN-7R | CCATAACCCTAGCAAGATTCAGAGC | SEQ ID 1226 |
| TPS9JL | 1 | TPS9JL-1F | TCAAGTTTTAGCCTCATCACAAAA | SEQ ID 1227 |
| TPS9JL | 1 | TPS9JL-1R | CCCAAATAGAAGGTTGATAAGTTGT | SEQ ID 1228 |
| TPS9JL | 2 | TPS9JL-2F | GCAGCGAGTTGACGAATTAAAGG | SEQ ID 1229 |
| TPS9JL | 2 | TPS9JL-2R | TGAAACCATAAATCCATGTTGGC | SEQ ID 1230 |
| TPS9JL | 3a | TPS9JL-3aF | TGCTTGATAACTGACATTCCCG | SEQ ID 1231 |
| TPS9JL | 3a | TPS9JL-3aR | AAGGTGAGTGGTGGTGAAAGC | SEQ ID 1232 |
| TPS9JL | 3b | TPS9JL-3bF | AGTGCTTGATAACTGACATTCCC | SEQ ID 1233 |
| TPS9JL | 3b | TPS9JL-3bR | ATGAAATGTAATGCCTAGCGTGG | SEQ ID 1234 |
| TPS9JL | 4 | TPS9JL-4F | TGCAAGAGATAGGATTGTGGAGC | SEQ ID 1235 |
| TPS9JL | 4 | TPS9JL-4R | TCTGCAACTGAGGCCAATGC | SEQ ID 1236 |
| TPS9JL | 5 | TPS9JL-5F | ACTTAAATTGTGCGGATCAACTACG | SEQ ID 1237 |
| TPS9JL | 5 | TPS9JL-5R | GTAAACTTTGTAACTTTCCTCCTTTCC | SEQ ID 1238 |
| TPS9JL | 6 | TPS9JL-6F | TCAGTGAAGCTCGATGGTTGC | SEQ ID 1239 |
| TPS9JL | 6 | TPS9JL-6R | TGCAATCTCTCATTATCTTGGGG | SEQ ID 1240 |
| TPS9JL | 7 | TPS9JL-7F | GAGATCATTCACCGTCTACCG | SEQ ID 1241 |
| TPS9JL | 7 | TPS9JL-7R | TTTTTGTCTCTTTTCCAACATGCG | SEQ ID 1242 |
| TPS9-like2JL | 1 | TPS9-like2JL-1F | TGTCGTCTCAAATCTTAGCAACC | SEQ ID 1243 |
| TPS9-like2JL | 1 | TPS9-like2JL-1R | ATGCAAAAATCGGTCTCCCC | SEQ ID 1244 |
| TPS9-like2JL | 2a | TPS9-like2JL-2aF | GCCAAGTTGAAGAATTGAAAGAAGTGG | SEQ ID 1245 |
| TPS9-like2JL | 2a | TPS9-like2JL-2aR | ACTCTCAAAATGATAGGACAATCCC | SEQ ID 1246 |
| TPS9-like2JL | 2b | TPS9-like2JL-2bF | AGAAGTGGTAAGAAAGGAGATATTTGG | SEQ ID 1247 |
| TPS9-like2JL | 2b | TPS9-like2JL-2bR | AAGAAACCCTATATCCATGTTGTCG | SEQ ID 1248 |
| TPS9-like2JL | 3a | TPS9-like2JL-3aF | GAATGTTTGGCGAGTGACACC | SEQ ID 1249 |
| TPS9-like2JL | 3a | TPS9-like2JL-3aR | AAGGGCCTCTCTAGGGCTCG | SEQ ID 1250 |
| TPS9-like2JL | 3b | TPS9-like2JL-3bF | AAGAACACCCCAATGATGATCC | SEQ ID 1251 |
| TPS9-like2JL | 3b | TPS9-like2JL-3bR | ACTAAGCTCCTTTTTGTGCATGG | SEQ ID 1252 |
| TPS9-like2JL | 4a | TPS9-like2JL-4aF | TGGTGGAAGGAATTAGACAGTGC | SEQ ID 1253 |
| TPS9-like2JL | 4a | TPS9-like2JL-4aR | ATCAGCGATTGAGGAAAGTGC | SEQ ID 1254 |
| TPS9-like2JL | 4b | TPS9-like2JL-4bF | TTATGAACCCCAATACTCTTTTGC | SEQ ID 1255 |
| TPS9-like2JL | 4b | TPS9-like2JL-4bR | AGGAGCTTATGTTCTTCAAATATACC | SEQ ID 1256 |
| TPS9-like2JL | 5 | TPS9-like2JL-5F | GGATAAACTCCATCCAGAATACTTGC | SEQ ID 1257 |
| TPS9-like2JL | 5 | TPS9-like2JL-5R | TCTTGTTCAAATTCCTCAAAAGATTGC | SEQ ID 1258 |
| TPS9-like2JL | 6a | TPS9-like2JL-6aF | GAAGCTCGATGGTTGAATGAAGG | SEQ ID 1259 |
| TPS9-like2JL | 6a | TPS9-like2JL-6aR | CGCTTGCCCTAACAATCTTGG | SEQ ID 1260 |
| TPS9-like2JL | 6b | TPS9-like2JL-6bF | TGATGGCTTGCTCTTTAGTTGG | SEQ ID 1261 |
| TPS9-like2JL | 6b | TPS9-like2JL-6bR | AGCCACGTCATCCATGTACC | SEQ ID 1262 |
| TPS9-like2JL | 7a | TPS9-like2JL-7aF | AGAATGAGCAAGAGAGAAATCATATACC | SEQ ID 1263 |
| TPS9-like2JL | 7a | TPS9-like2JL-7aR | CCATGCAATAACCACTCGCC | SEQ ID 1264 |
| TPS9-like2JL | 7b | TPS9-like2JL-7bF | TAGGCGAGTGGTTATTGCATGG | SEQ ID 1265 |
| TPS9-like2JL | 7b | TPS9-like2JL-7bR | CCTAGCAAGATTCAGAGCACG | SEQ ID 1266 |
| TPS9-likeJL | 1 | TPS9-likeJL-1F | AAAATGAGAAAATACATAAAATTGTTCGAC | SEQ ID 1267 |
| TPS9-likeJL | 1 | TPS9-likeJL-1R | TCGATCTCCCCAAATAGATGGA | SEQ ID 1268 |
| TPS9-likeJL | 2 | TPS9-likeJL-2F | TTGAAGAATTGAAAGAAGTAGTAAGAAAGG | SEQ ID 1269 |
| TPS9-likeJL | 2 | TPS9-likeJL-2R | TCAAAATGATAGGACAATCCCAAACG | SEQ ID 1270 |
| TPS9-likeJL | 3a | TPS9-likeJL-3aF | TGACAAGTTCAAAGATGAGAATGGC | SEQ ID 1271 |
| TPS9-likeJL | 3a | TPS9-likeJL-3aR | TAGTAAATCTTCCCCGACGC | SEQ ID 1272 |
| TPS9-likeJL | 3b | TPS9-likeJL-3bF | ACATTTGAGTTGCGTCGGGG | SEQ ID 1273 |
| TPS9-likeJL | 3b | TPS9-likeJL-3bR | TAGCTTGGAGCCTGTTTAGGG | SEQ ID 1274 |
| TPS9-likeJL | 3c | TPS9-likeJL-3cF | AAGAAAAACCCTAAACAGGCTCC | SEQ ID 1275 |
| TPS9-likeJL | 3c | TPS9-likeJL-3cR | CTAAGCTCCTTTTTGTGCATGG | SEQ ID 1276 |
| TPS9-likeJL | 4 | TPS9-likeJL-4F | AGAGACAGGAGTGTGGAACTATACC | SEQ ID 1277 |
| TPS9-likeJL | 4 | TPS9-likeJL-4R | TGTCATCAGCTATTGTGGCAAAGG | SEQ ID 1278 |
| TPS9-likeJL | 5 | TPS9-likeJL-5F | TGAAGAATTTGAGCAAGGGCTTA | SEQ ID 1279 |
| TPS9-likeJL | 5 | TPS9-likeJL-5R | GCGTAATGAACTCTGTAAGTTTCT | SEQ ID 1280 |
| TPS9-likeJL | 6 | TPS9-likeJL-6F | GTGGTTAAAGAAAGCTGAACGC | SEQ ID 1281 |
| TPS9-likeJL | 6 | TPS9-likeJL-6R | GCCGACAAATAGTAGTGGATGC | SEQ ID 1282 |
| TPS9-likeJL | 7 | TPS9-likeJL-7F | TGAATAGGCGAGTGGTTATTGC | SEQ ID 1283 |
| TPS9-likeJL | 7 | TPS9-likeJL-7R | TGCAAGATTCAGGGCACG | SEQ ID 1284 |

The hybridization/melting characteristics of the primers of Table 2 are shown in FIG. 1. The designed primers all had melting temperatures between 57° C. and 63° C., with annealing temperatures ranging from 52° C. to 58° C.

These primers can be used to amplify their target sequences in traditional PCR on total DNA or purified genomic DNA, as well as in reverse-transcriptase PCR to identify variants and presence/absence variation of expressed DNA.

Table 3 depicts an example of parameters of a traditional PCR protocol in which the primers provided herein (e.g., as described in Table 2 and FIG. 1) can be used:

TABLE 3

| Traditional Program: 30 cycles | Temperature | Time | |
|---|---|---|---|
| Initiation/Polymerase Activation: | 95-97° C. | 2 | mins |
| Start of Cycling: | | | |
| Denaturation: | 95° C. | 10 | secs |
| Step Down Annealing: | 52-58° C. ** | 10 | secs |
| Elongation: | 72° C. | 30 | secs |
| Final Elongation Step: | 72° C. | 5-15 | mins |

** Annealing temperature is specific for each primer set. For determining optimal T_{annealing}, the highest Tₘ of the two primers in a given set minus 5 degrees Celsius results in the optimal annealing temperature.

Table 4 depicts an example of parameters of a step-down PCR protocol, using standard PCR reagents, in which the primers provided herein (e.g., as described in Table 2 and FIG. 1) can be used:

TABLE 4

| Step-Down Program: 30 cycles | Temperature | Time | |
|---|---|---|---|
| Initiation/Polymerase Activation: | 95-97° C. | 2 | mins |
| Start of Cycling: | | | |
| Denaturation: | 95° C. | 10 | secs |
| Step Down Annealing: | 60° C. (sec. | 10 | secs |
| | target 50° C.) | | |
| Elongation: | 72° C. | 30 | secs |
| Final Elongation Step: | 72° C. | 5-15 | mins |

It is understood by those of skill in the art that modifications to these protocols can be made to achieve the same or similar results. For example, the temperatures for the various steps in the can be modified by between about 1-5° C., or touchdown PCR can be performed, i.e., the annealing temperature is adjusted based on the cycle number.

### Example 2

Use of Exon-Specific Primers to Obtain Terpene Synthase Fingerprints of a Plant Cultivar

DNA Isolation

Genomic DNA is isolated from Cannabis samples using the Qiagen DNA Easy Plant genomic DNA isolation kits (Qiagen) or the Promega Wizard genomic DNA kit (Promega) using manufacturer's instructions, FTA plant saver cards (Whatman's Flinders Technology Associates, a technology developed by GE Healthcare for lysing cells and storing DNA on a piece of Whatman filter paper), or an in-house crude preparation of genomic DNA extracts. A crude DNA extract is prepared by Tris/Triton-X pre-treatment of 1 mm raw leaf or leaf imprinted FTA card sections, as modified from Klimyuk et al. (Plant J., 3(3):493-494 (1993)) in a modified 96 well format for high throughput processing. Leaf or FTA selections were placed aseptically in a 96 well microtiter plate, 100 uL 0.25M Tris-HCI with 0.25% Triton-X-100 was added to each well, and the plates were incubated at 100° C. for 5 minutes on a Veriti thermocycler (ABI). 3uL of crude genomic DNA extract was used as input for the pre-amplification PCR reaction.

RNA Isolation

Plant material/tissue from Cannabis is flash frozen in liquid nitrogen or placed in a RNase inhibitory solution, such as RNALater, for in-grow collection of tissue for cDNA analysis. Total RNA is then isolated from Cannabis tissues using any plant RNA extraction kit or method available and/or known to those of skill in the art. Examples of such kits include: the Qiagen RNAeasy plant extraction kit, which can be used following manufacturer's instructions or modifying the instructions by using RLC buffer to provide a higher quality extraction that yields greater concentrations of RNA, the Direct-zol RNA isolation kit and the Zymogen Quick-RNA Plant mini prep kit. An example of an RNA isolation protocol is as follows:

Total RNA is isolated from fresh Cannabis leaf tissue samples using the Direct-zol RNA isolation kit and Zymogen Quick-RNA Plant mini prep kit with DNAase digestion, using manufacturer's instructions (Zymogen). Purified RNA is prepared for quantification using the QuantiFluor HS-ssRNA ssRNA System (Promega) and quantified using a Quantus Fluorometer (Promega), as per the manufacturer's instructions.

The quantified RNA is diluted to a final working concentration of 5ng/uL and used as normalized input into either a First strand cDNA synthesis reaction or a one-step reverse transcriptase real-time qPCR reaction.

cDNA Synthesis

The single-stranded RNA is then converted to double-stranded cDNA using any available cDNA-synthesis reverse transcriptase (RT-PCR) kit or method available and/or known to those of skill in the art. Example of kits that can be used are the High Capacity RNA-to-cDNA Kit or The SuperScript IV First-Strand Synthesis System (both from Thermofisher Scientific), or Qiagen's FastLane Cell cDNA Kit. These provide double-stranded DNA that can be subjected to High Resolution Melt (HRM) analysis with or without a pre-amplification step, depending on the RNA extraction quality. Additionally, after the RNA extraction and before the cDNA synthesis, the sample can be subjected to ribo-depletion or mRNA amplification, to remove rRNA and obtain greater sensitivity for the detection of terpene synthase genes that are expressed at a low level. An example of an cDNA synthesis protocol is as follows:

Quantified RNA is used as input for cDNA synthesis using the SuperScript™ IV First-Strand Synthesis System (ThermoFisher). cDNA synthesis reactions are prepared as follows: 1 μL 50 μM Oligo d(T) 20 primer, 1 μL of 10 mM dNTP mix (10 mM each dNTP), 8 μLTemplate RNA (10 pg-5 μg total RNA or 10 pg-500 ng mRNA), up to 3 μL DEPC-treated water are mixed together for a 13 μL final volume. After mixing and briefly centrifuging, the RNA-primer mix reactionsawere heated at 65° C. for 5 minutes, and then incubated at 0° C. for 2 minutes on a Veriti thermocycler (ABI).

Following annealing, the plate is pierced using a plate piercer and 7 μL Reverse transcriptase (RT) reaction mix is added to each reaction for a final volume of 20 μL final volume for cDNA synthesis. The RT reaction mix is prepared by mixing together the following: 4 μL of 5× SSIV Buffer, 1 μL of 100 mM DTT, 1 μL of Ribonuclease Inhibitor, and 1 μL of SuperScript™ IV Reverse Transcriptase (200 U/μL). The plate is sealed and briefly centrifuged, then loaded onto a Veriti thermocycler for cDNA synthesis using the following protocol:

Incubate the combined reaction mixture at 50-55° C. for 10 minutes;

Inactivate the reaction mixture by incubation at 80° C. for 10 minutes, then store at 4° C.

The resulting products of cDNA synthesis are prepared for quantification using the QuantiFluor HS-dsDNA System (Promega) and quantified using a Quantus Fluorometer (Promega), as per the manufacturer's instructions. The quantitated cDNA is diluted to a 2 ng/uL final working concentration and used as normalized input into either an end-point PCR reaction or a Taqman real-time qPCR reaction.

Endpoint PCR with Gel Analysis

2.5 uL of normalized cDNA is used as input for a PCR Master Mix (total volume: 22.5 uL), as follows: 12.5 uL 2× Promega Colorless GoTaq (Promega), 0.1 uL of 100 uM Primer

Mix (see Table 2 in Example 1 for primer sequences; 100 uM of a single primer pair to detect one exon, or multiple primer pairs that each detects an exon of unique size in the set of TPS genes of the plant cultivar of interest), and 9.5uL Nuclease free Water (Ambion).

The reactions are subjected to the following thermocycler protocol: 1 cycle of 95° C. for 10mins; 35 cycles of 95° C. for 40 seconds, 60° C. for 2 mins, 72° C. for 2 mins; 1 cycle of 72° C. for 5mins; 4° C. hold. The End point PCR reactions are analyzed by diluting 1:2 in nuclease free water and 20u1 is loaded into each well of one or more E-Gel™ EX Agarose Gels, 2%, depending on the number of samples, and run for 10 minutes on 1-2% gel settings for the E-gel system. The bands are analyzed for the presence of exons, based on the expected sizes of the amplicons. In addition, if the DNA is normalized, the intensities (e.g., fluorescence intensity) of the bands can provide information regarding the numbers of copies of the TPS genes and/or the ploidy (e.g., diploid, triploid, tetraploid, etc.).

Pre-Amplification PCR

To reduce the effect of plant materials in subsequent reactions and analyses, for example when crude DNA or RNA extracts are used, plant pigments and potentially real time qPCR-inhibiting compounds often found in such extracts can optionally be removed by performing a pre-amplification PCR for 10 cycles. 2.5 uL of crude genomic DNA extract is transferred to a second PCR plate, with each well pre-loaded with 22.5 uL of Pre-amplification PCR master mix prepared per reaction as follows: 12.5 uL 2× Promega Colorless GoTaq (Promega), 3 uL of 4 uM of the desired primers to be analyzed (see Table in Example 1 for primer sequences), and 7 uL Nuclease free Water (Ambion). The reactions are subjected to the following thermocycler protocol: 1 cycle of 95° C. for 10mins; 10 cycles of 95° C. for 40 sec, 60° C. for 2 mins, 72° C. for 2 mins; 1 cycle of 72° C. for 5 mins; 4° C. hold.

Pre-amplification reactions are diluted 1:5 with 100 uL Nuclease free water (Ambion). The diluted pre-amplification reactions are prepared for quantification using the QuantiFluor dsDNA System (Promega) and quantified using a Quantus Fluorometer (Promega), as per manufacturer's instructions. Quantitated diluted pre-amplification reactions reveal a final working concentration of ˜1 ng/uL, which is used as unnormalized input into the real time qPCR reactions.

Hicih Resolution Melt (HRM) Analysis

HRM analysis was performed in 10 uL reactions on a LightCycler 480 qPCR (Roche Applied Systems) using the following protocol: 1 pre-incubation cycle (95° C. for 10 mins), 45 amplification cycles (95° C. for 10 secs, 60° C. for 15 secs, 72C for 10 secs), 1 cycle of HRM (95° C. for 1min, 40° C. for 1 min, 65° C. for 1 sec and heat to 95° C. with 25 continuous acquisitions per degree Celsius followed by a final cooling cycle (40° C. for 10 secs) (Vossen et al., Biochemica 4:10-11 (2007)). Each reaction contains: 5 uL of ˜1 ng/uL of the diluted pre-amplified template that is the product of pre-amplification PCR. cDNA also can be amplified in an exon-specific manner to get a fingerprint of the terpene synthase expressosome, i.e., to see which of the TPS genes are expressed (using a single pair of primers or multiple pairs of primers that each amplify an exon of a unique size), or gDNA can be amplified (with or without pre-amplification) to get a fingerprint of the entire genome.

5 uL of HRM Master Mix (prepared per reaction as follows: 3.5 uL 2× High Resolution Melting Master Mix containing HRM dye (Roche Applied Systems), 0.6 uL of 4 uM Primer Mix (see Table 2 in Example 1 for primer sequences; 4 uM of a single primer pair to detect one exon, or multiple primer pairs that each detects an exon of unique size in the set of TPS genes of the plant cultivar of interest), 0.8 uL of 25 mM MgCl2, 1.125 uL of Nuclease free water). High Resolution Melting data was analyzed using the LightCycler 480 Melt Genotyping software. Fluorescence intensity as a function of temperature for each sample also was analyzed using R software and Matlab custom scripts to determine statistical variation of melt curves and statistical analysis. An example of a resulting fingerprint representing 24 terpene synthase paralogs (i.e., a subset of the 74 detectable genes) of a Cannabis plant cultivar is provided below:

As shown in the last column of the Table above, each of the detected 24 TPS genes is associated with/assigned a unique barcode based on the exons within which amplicons are amplified and detected for each gene. In addition, each amplicon within an exon that is targeted for amplification (each yellow square, will be changed to black/white indicator for the application) can be assigned a genotypic group number based on the HRM group assigned to that amplicon during the HRM analysis. The combination of all the barcodes that are detected provides an overall cultivar fingerprint (TPS gene profile) of the Cannabis plant cultivar; this fingerprint is unique to the cultivar and depends on the terpene synthase genes and/or paralogs that are present and any mutations they carry, which can be identified by HRM.

The fingerprint obtained using HRM can encompass all 74 terpene synthase paralogs that can be amplified using the primers listed in Table 2 provided in Example 1, e.g., for a complete understanding of these terpene synthase paralog genotypes present in a given cultivar's genome. Alternately, smaller subsets of these primers can be used, depending on the terpene synthase(s) or terpene(s) of interest.

### Example 3

Use of Terpene Synthase Fingerprints of a Plant Cultivar for Selective Breeding

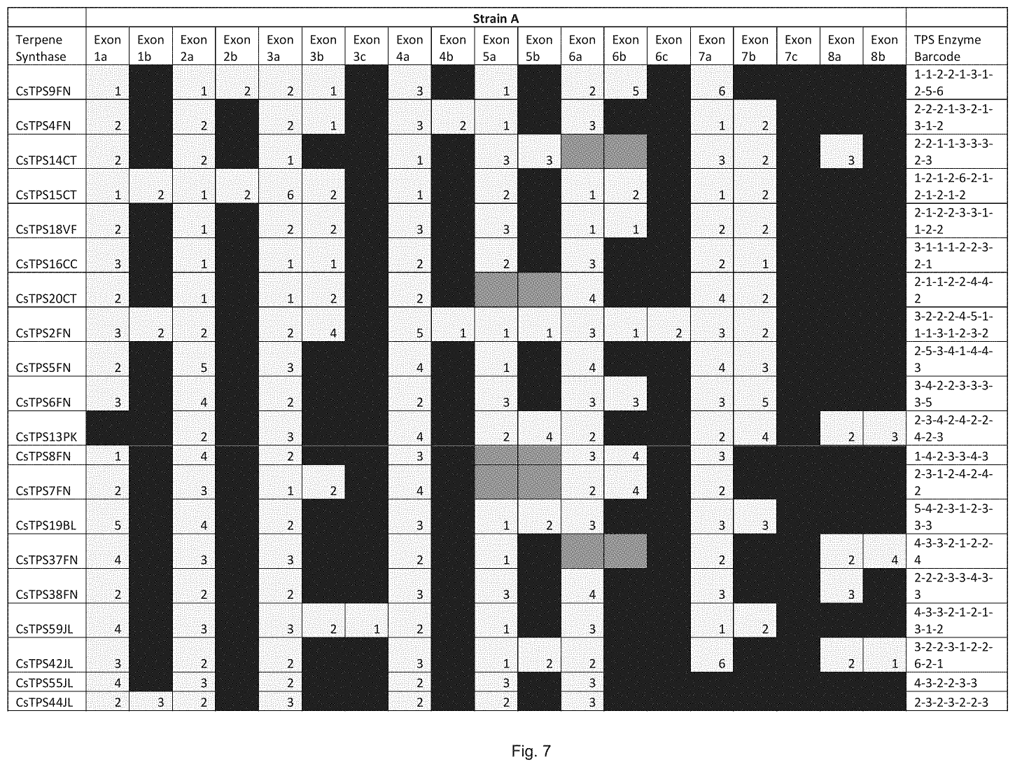

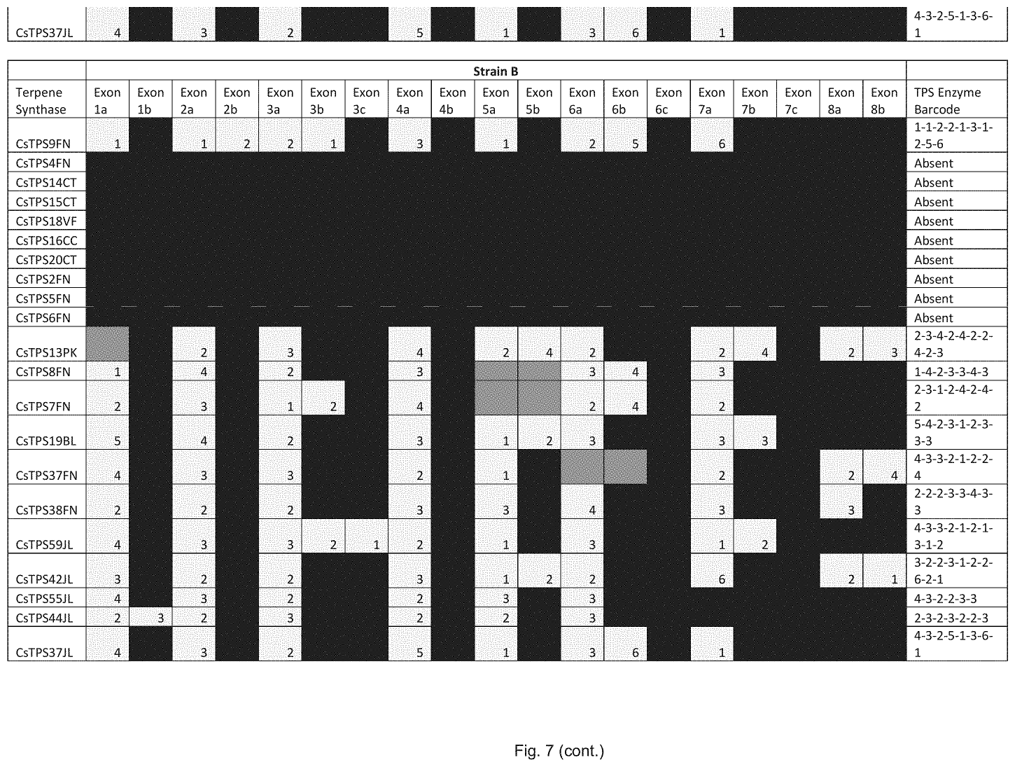

The HRM analysis described in Example 2 above can be used to obtain terpene synthase paralog fingerprints of plant cultivars and use that information for selective breeding of offspring cultivars having desired characteristics. For example, provided in FIG. 7 are two “parent” plant cultivars, Strain A and Strain B, having fingerprints for 21 terpene synthase paralogs as shown in FIG. 7.

The fingerprints (barcodes) for Strain A (chemovar) and Strain B (chemovar) show that they are homozygous for terpene synthase paralogs 1 and 11-21. Terpene synthase paralogs 2-10 are present in Strain A and absent in Strain B. Therefore, by breeding Strain A with Strain B, one would knowingly be in-breeding for homozygosity of paralogs 1 and 11-21, which are seen in both strains (chemovars) as demonstrated by the identical enzymatic bar codes. The offspring should be homozygous for these paralogs and one would expect that if both parents expressed a given gene, then the offspring also will express that gene.

With respect to the paralogs that are absent in Strain B but present in Strain A, one can expect that some of the offspring might inherit the paralogs that are present in Strain A. With this knowledge, one can screen the offspring with only the primers for the paralogs that one wants to ensure are either inherited or not inherited in the offspring, thereby breeding for and selecting offspring with desired characteristics.

### Example 4

Analysis of the Variation in TPS Gene Content Among Cannabis cultivars

To assess the extent of variation in TPS gene content among Cannabis varieties, six genome assemblies were used as shown in Table 6 below.

TABLE 6

| | | Assembly | | | |
|---|---|---|---|---|---|
| Name | Accession No. | Length (bp) | # Contigs | Contig N50 | |
| Jamaican Lion | GCA_003660325.2 | 1,073,903,735 | 556 | 3.8 | Mbp |
| Tibet(JL) | GCA_013030365.1 | 812,525,420 | 483 | 83 | Mbp |
| cs10 | GCF_900626175.1 | 876,147,649 | 221 | 92 | Mbp |
| Purple Kush | GCA_000230575.5 | 891,964,663 | 12,836 | 133 | Kbp |
| USO31 | PGA_Assembly_uso31.fasta | 982,000,000 | 761 | 103 | Mbp |
| cannbio2 | GCA_900626175.2 | 914,397,426 | 147 | 91 | Mbp |

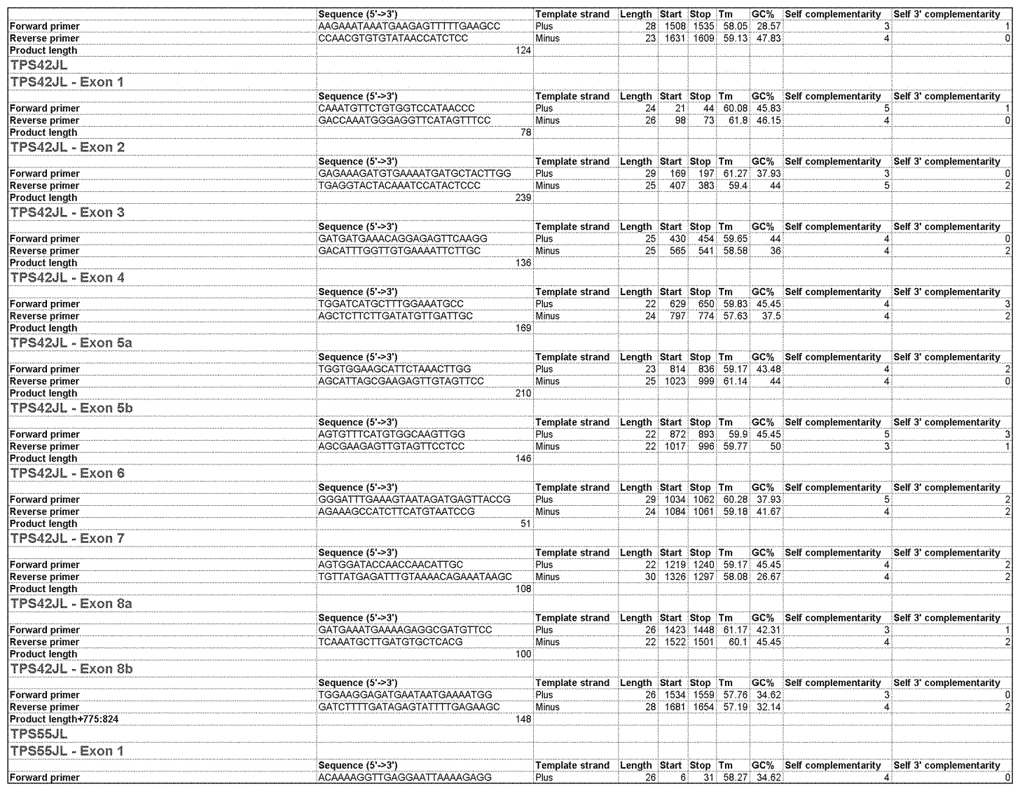

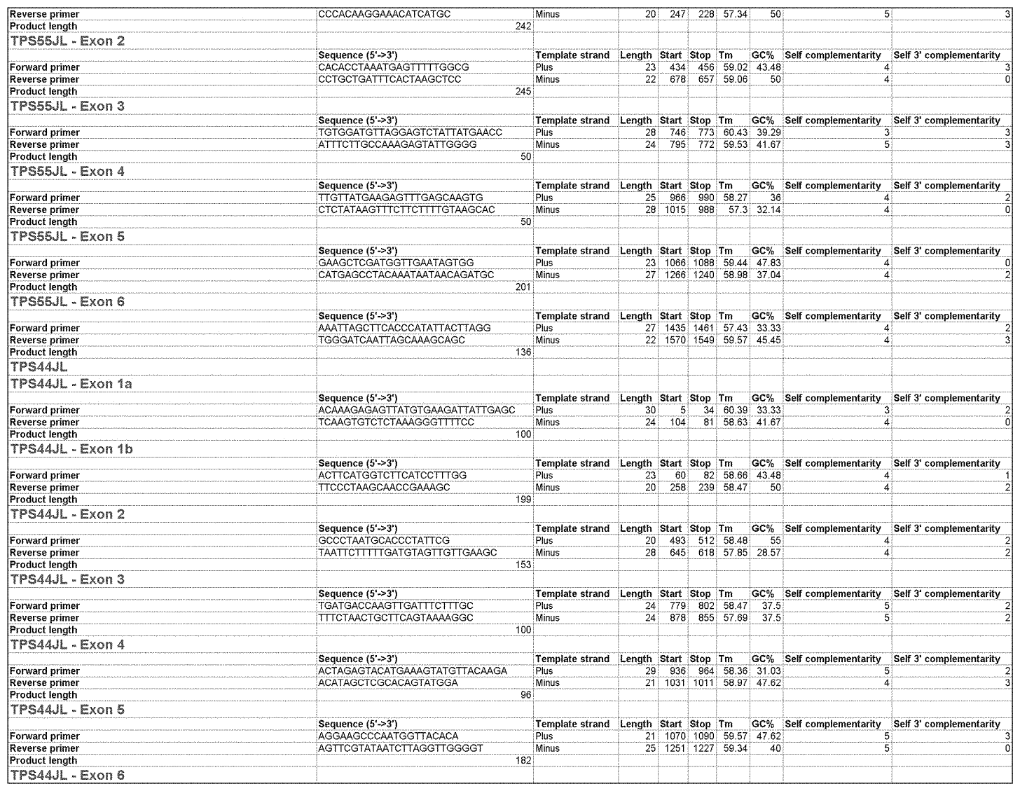

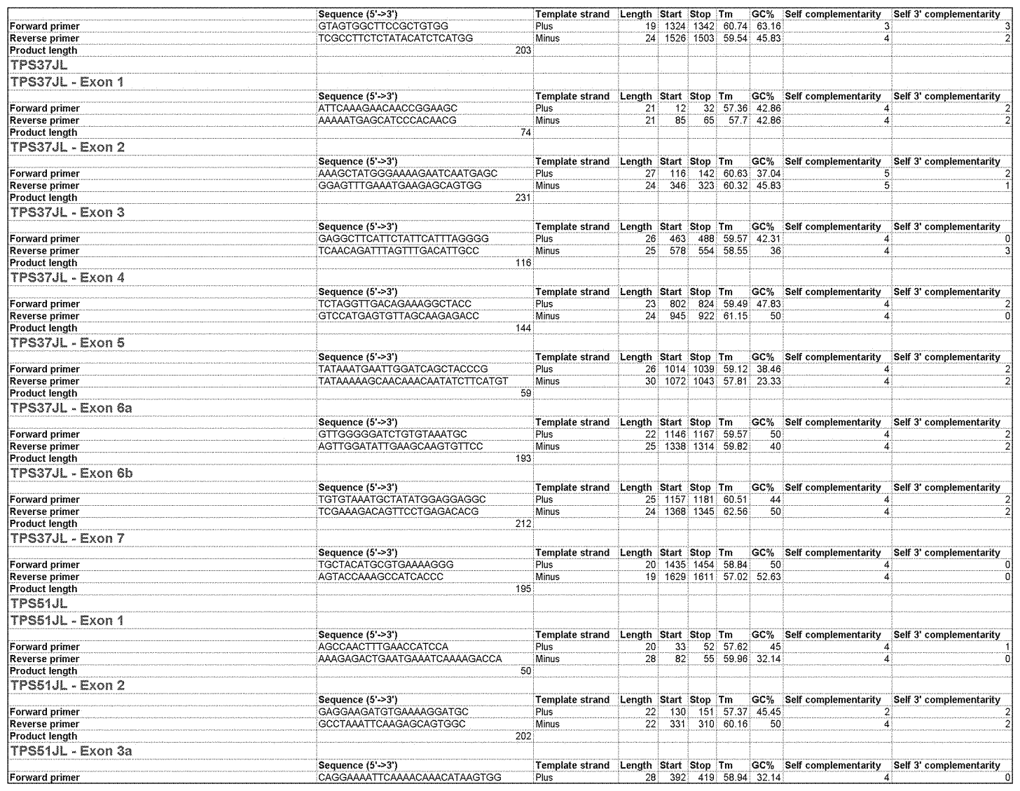

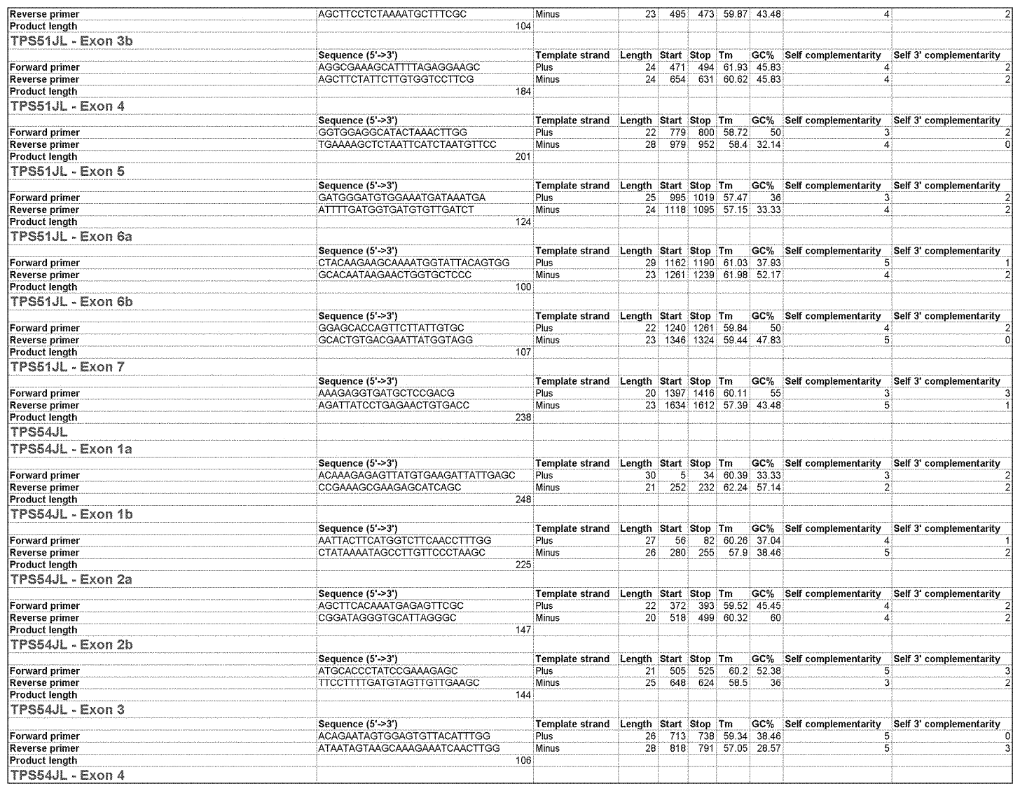

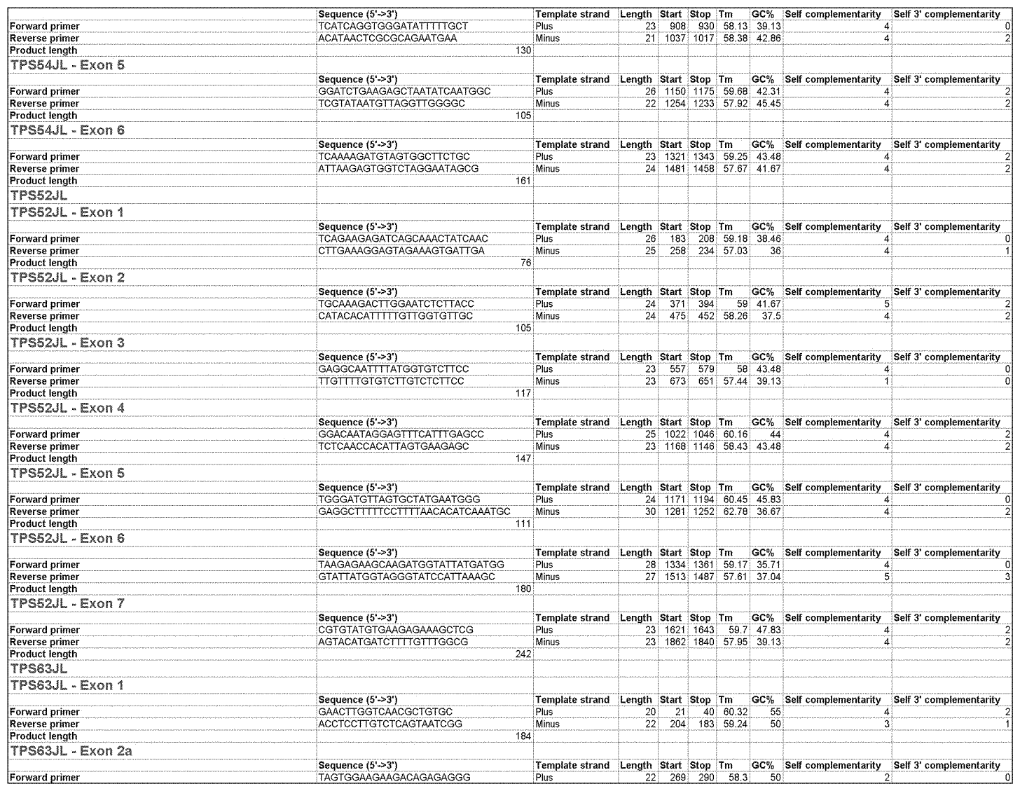

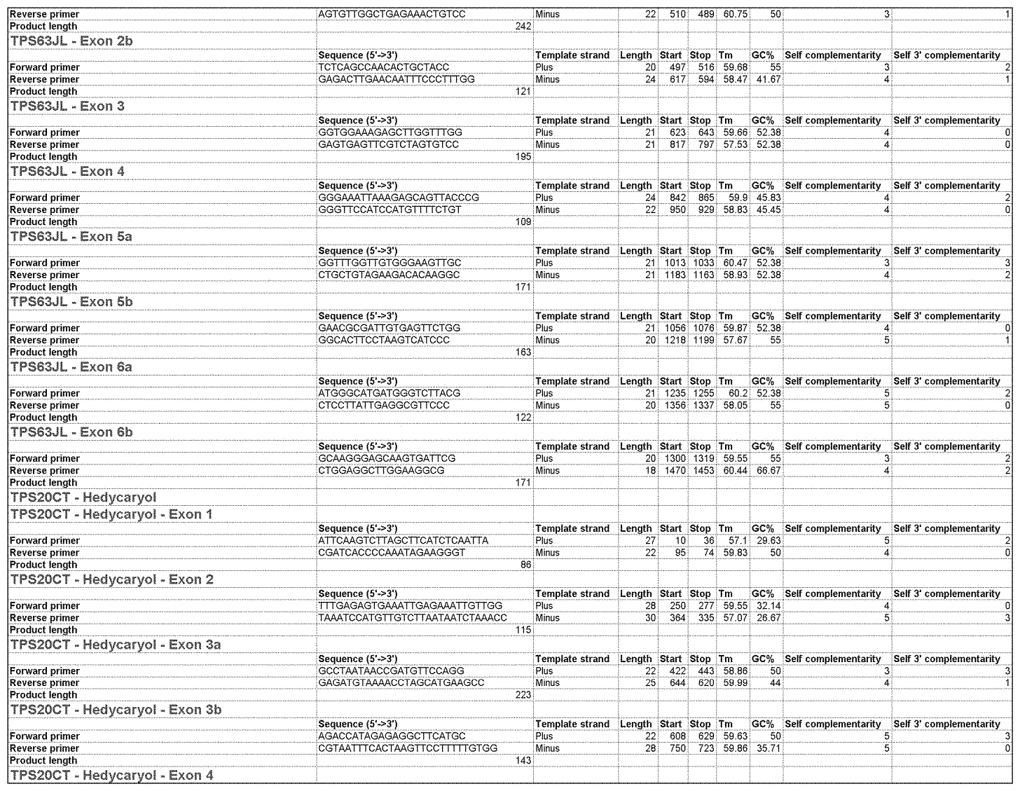

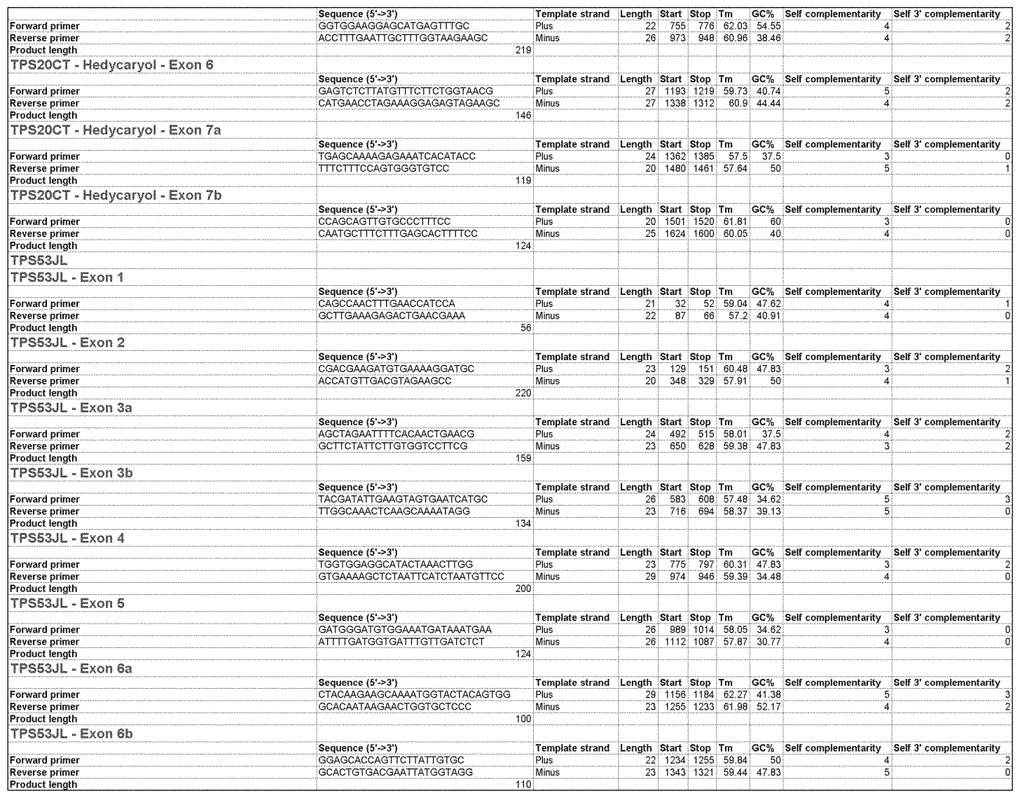

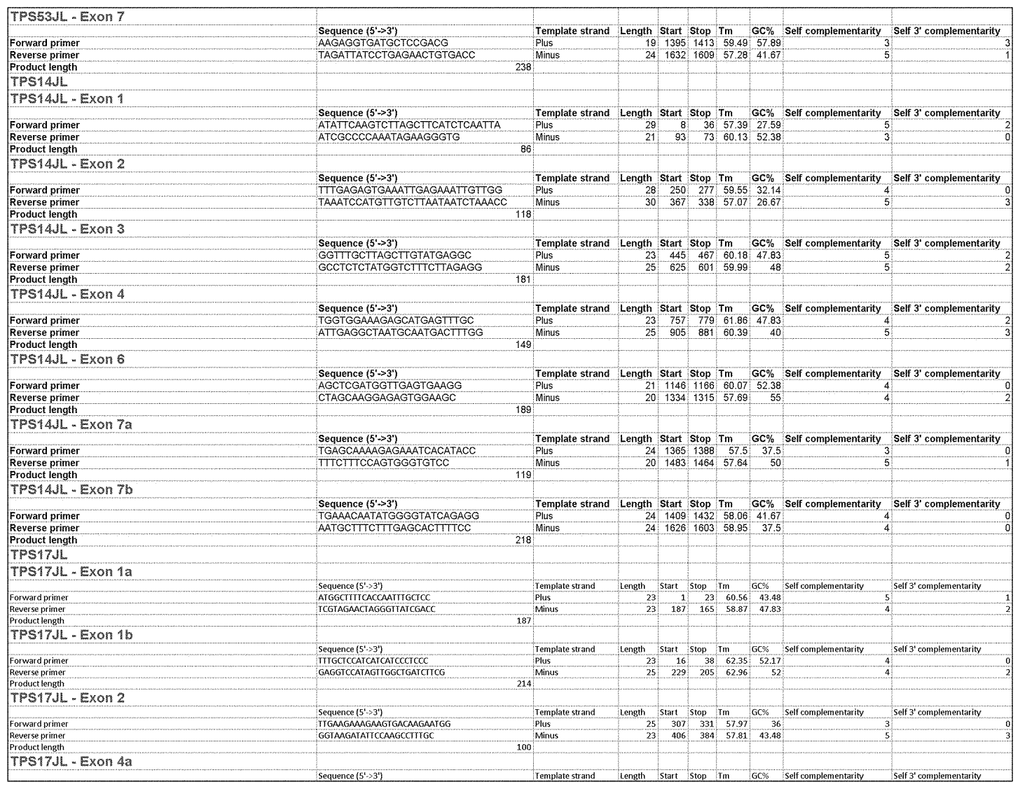

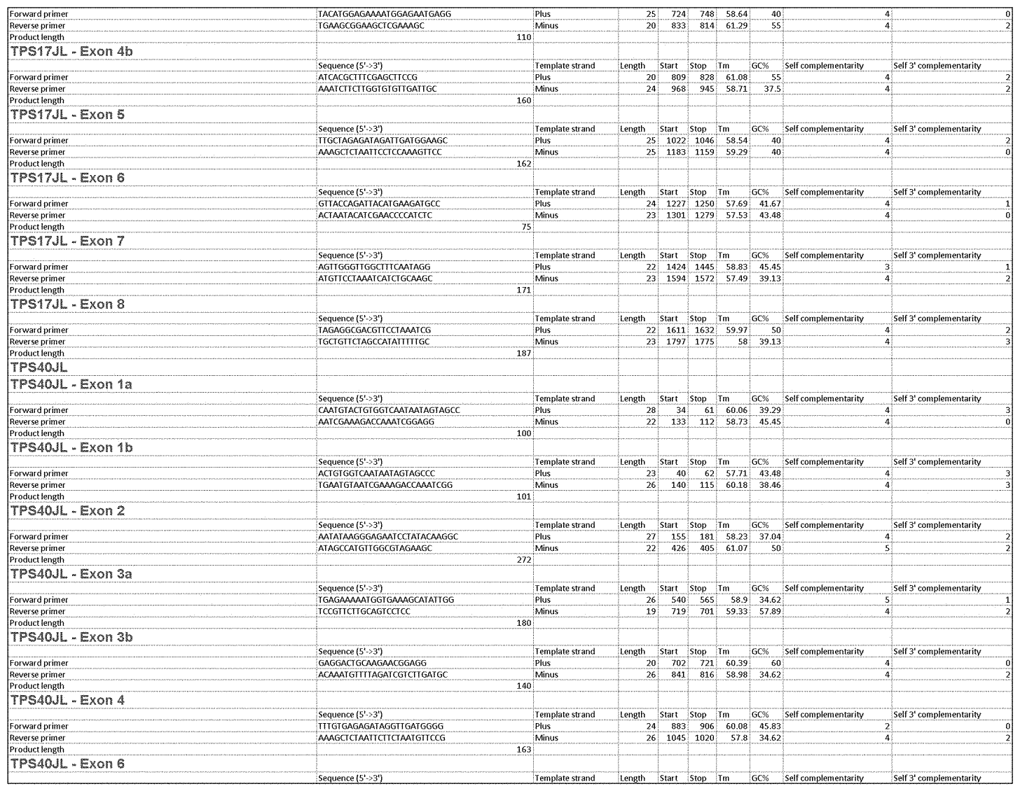

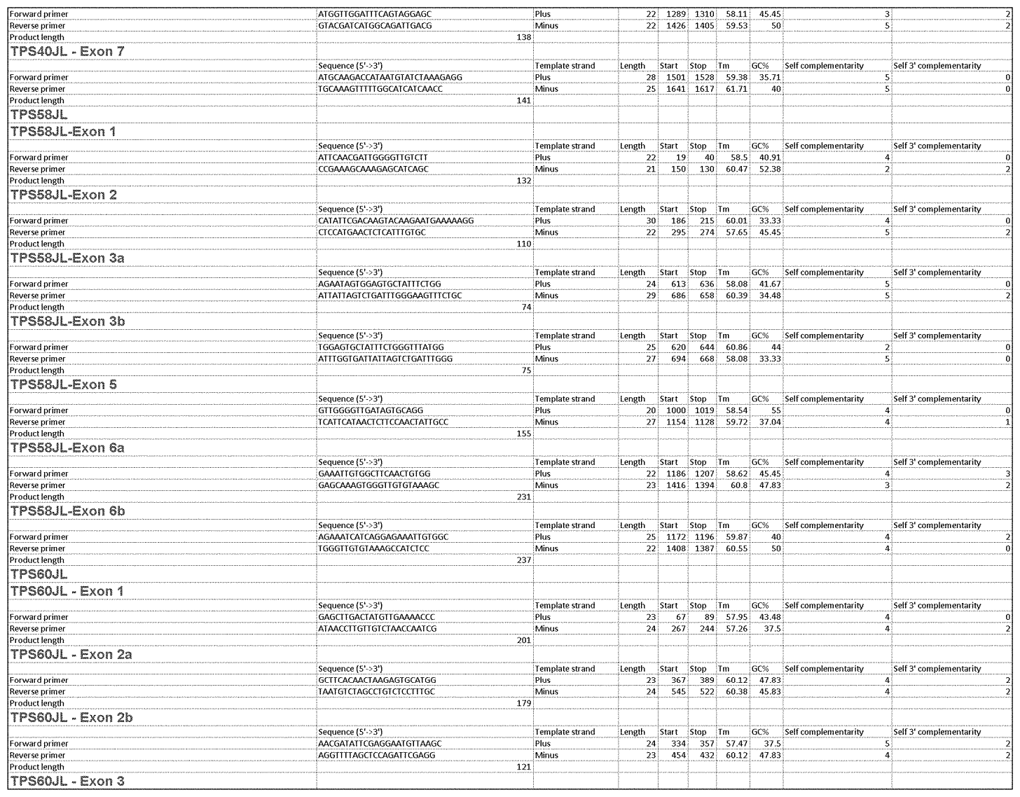

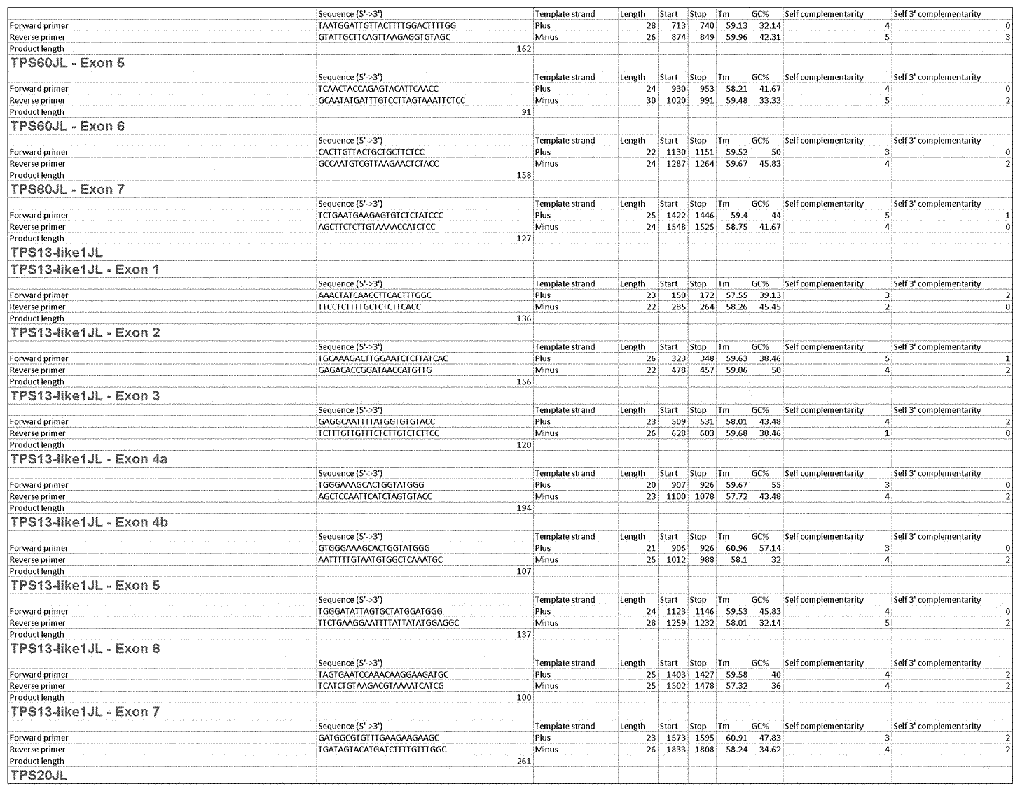

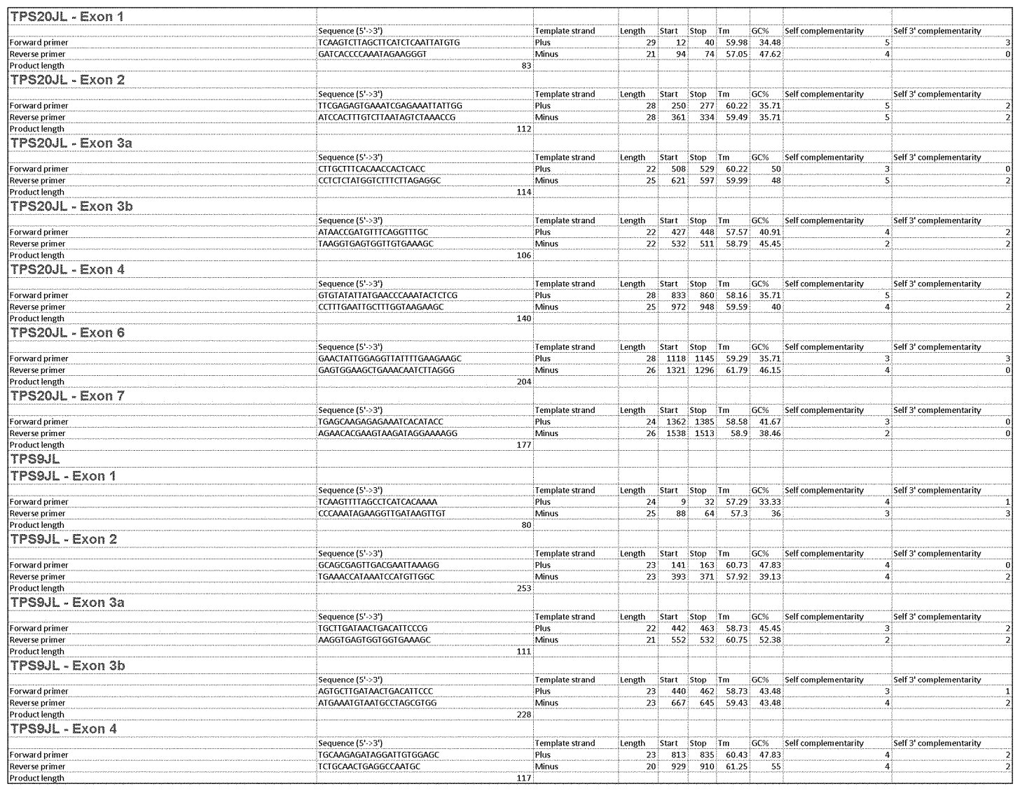

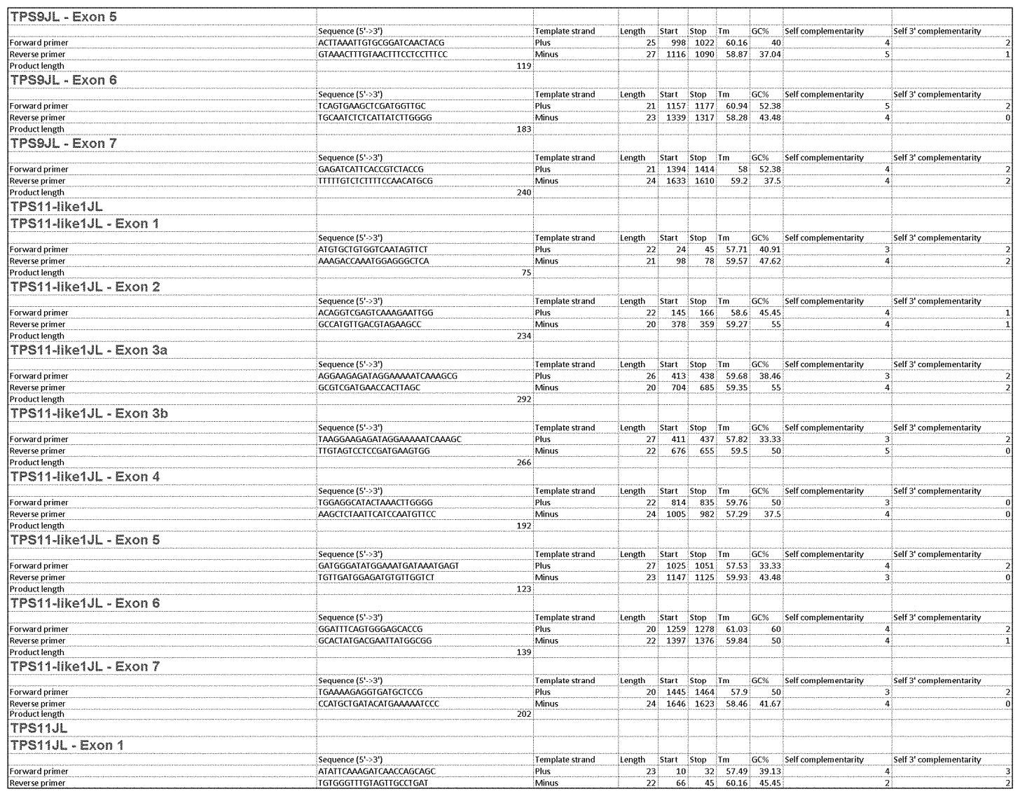

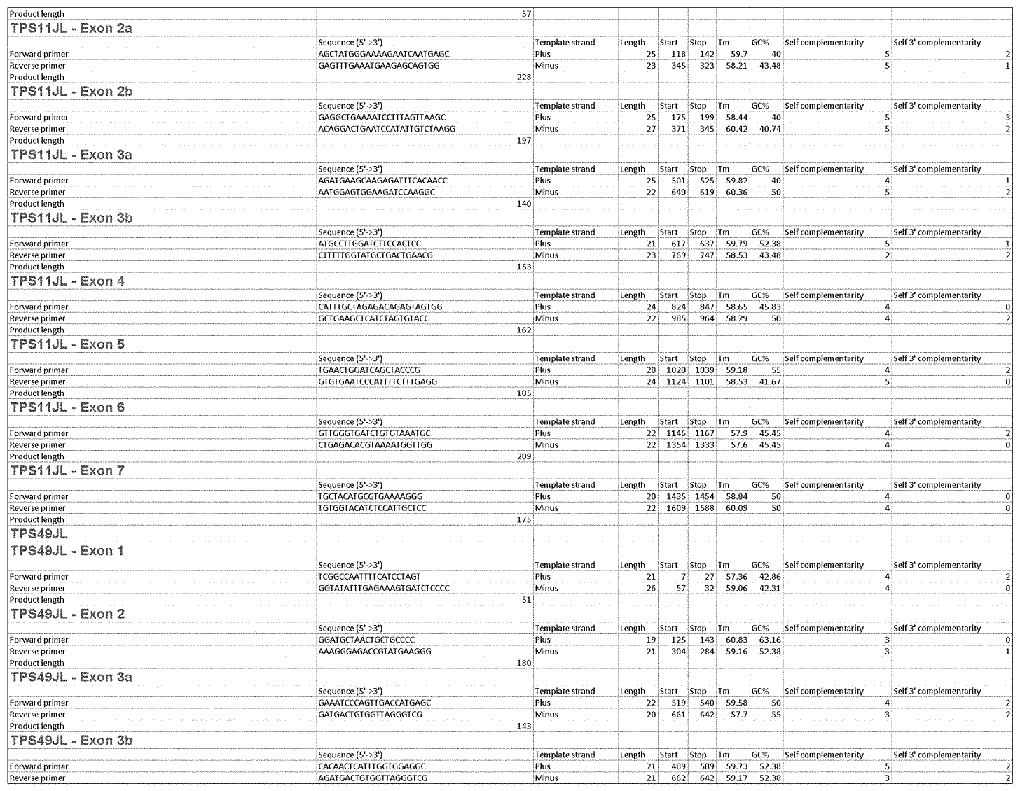

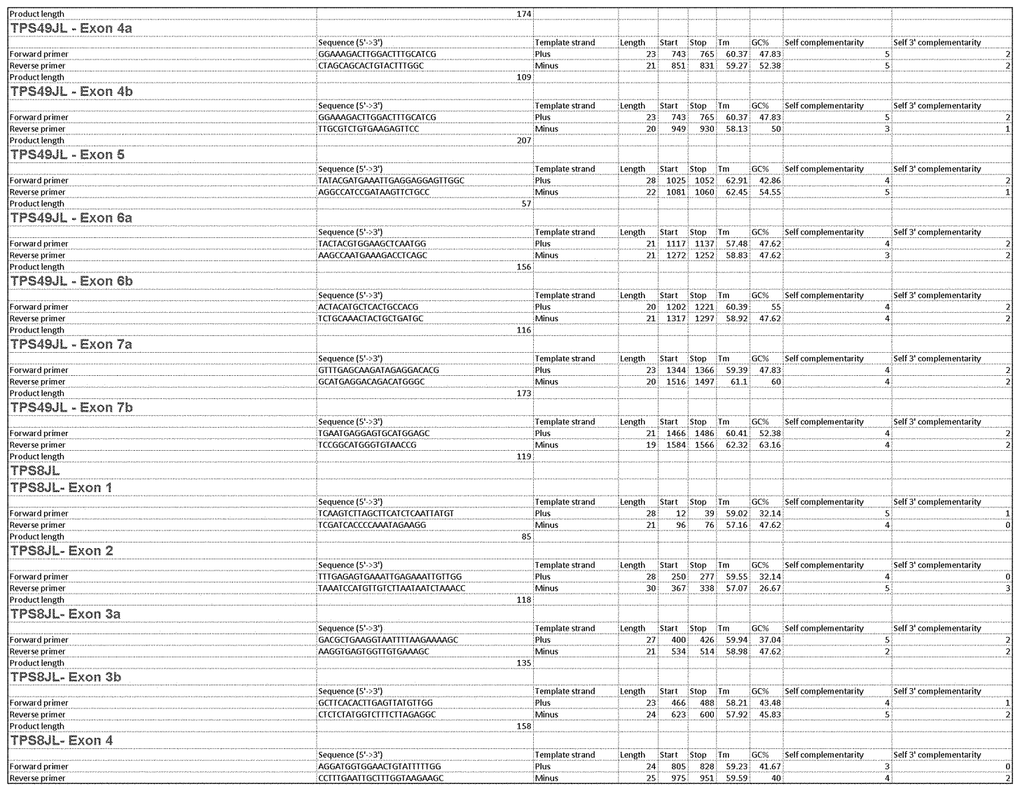

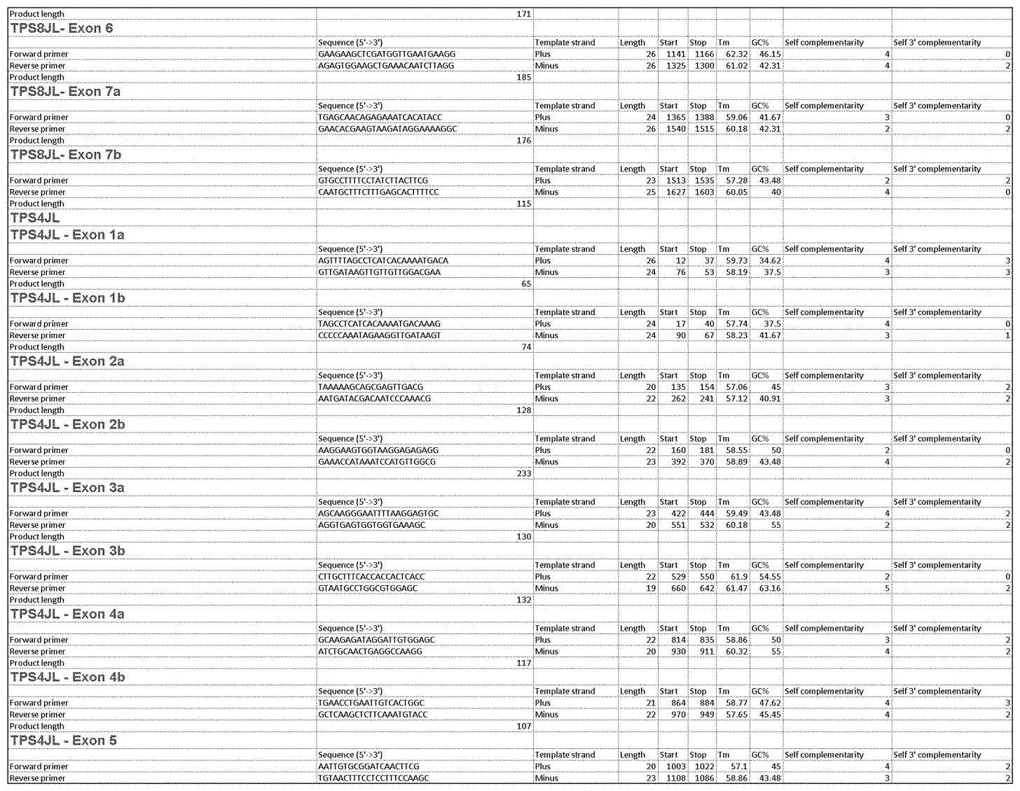

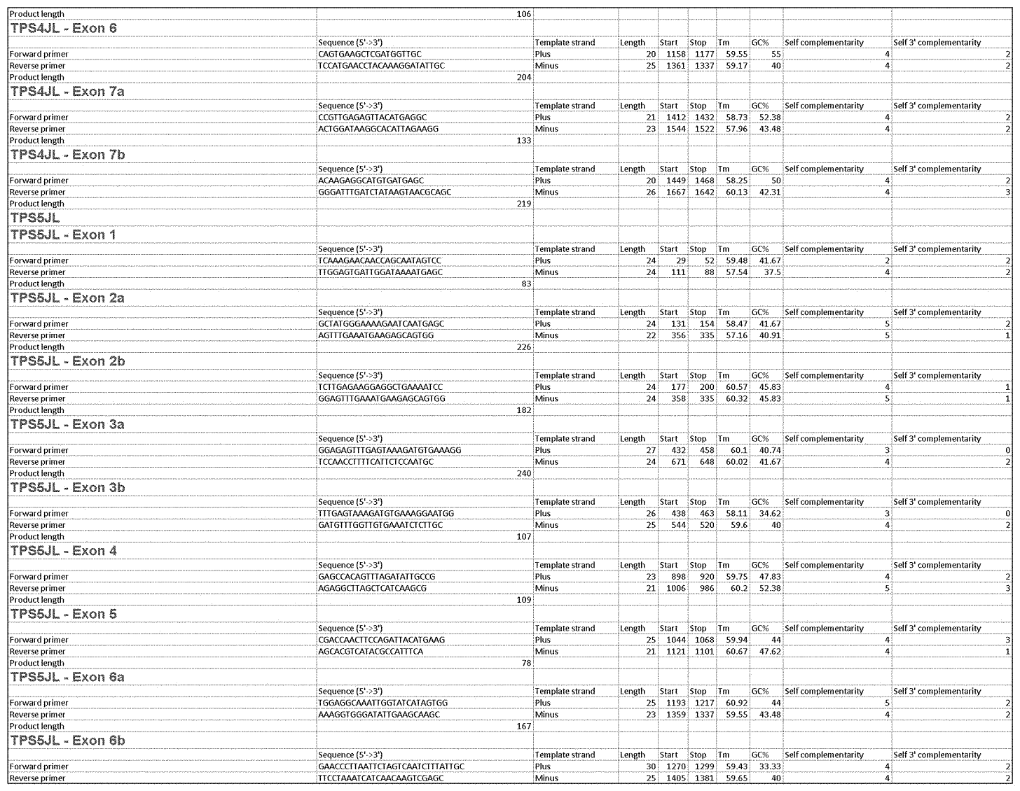

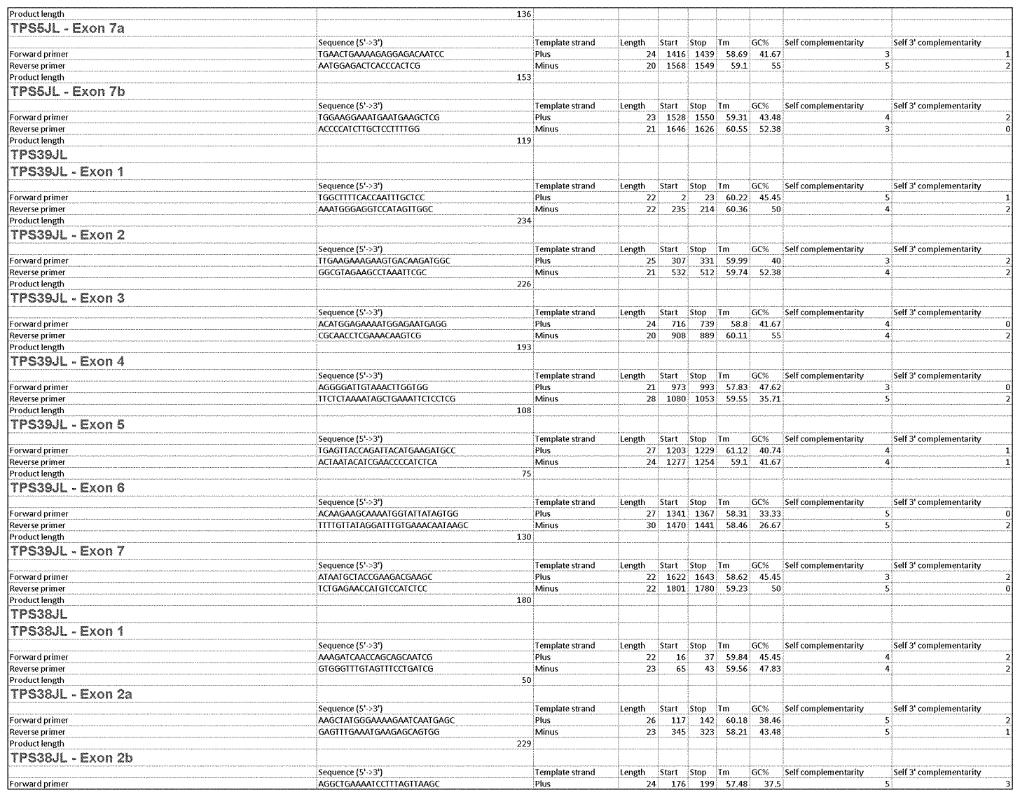

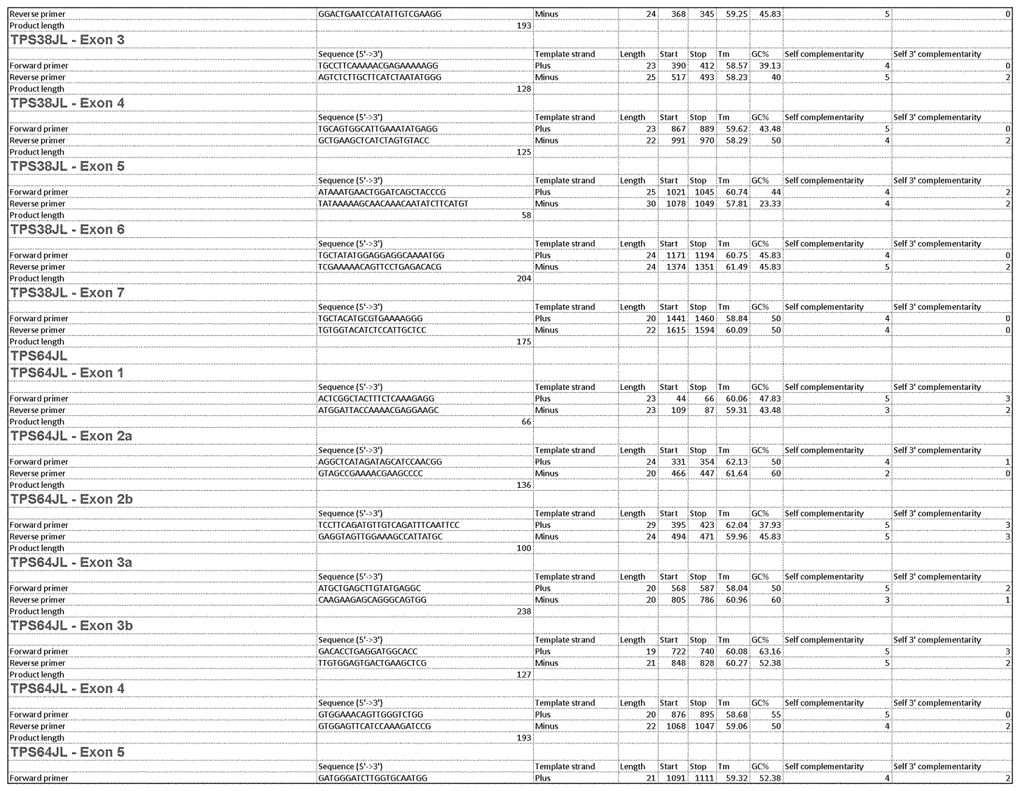

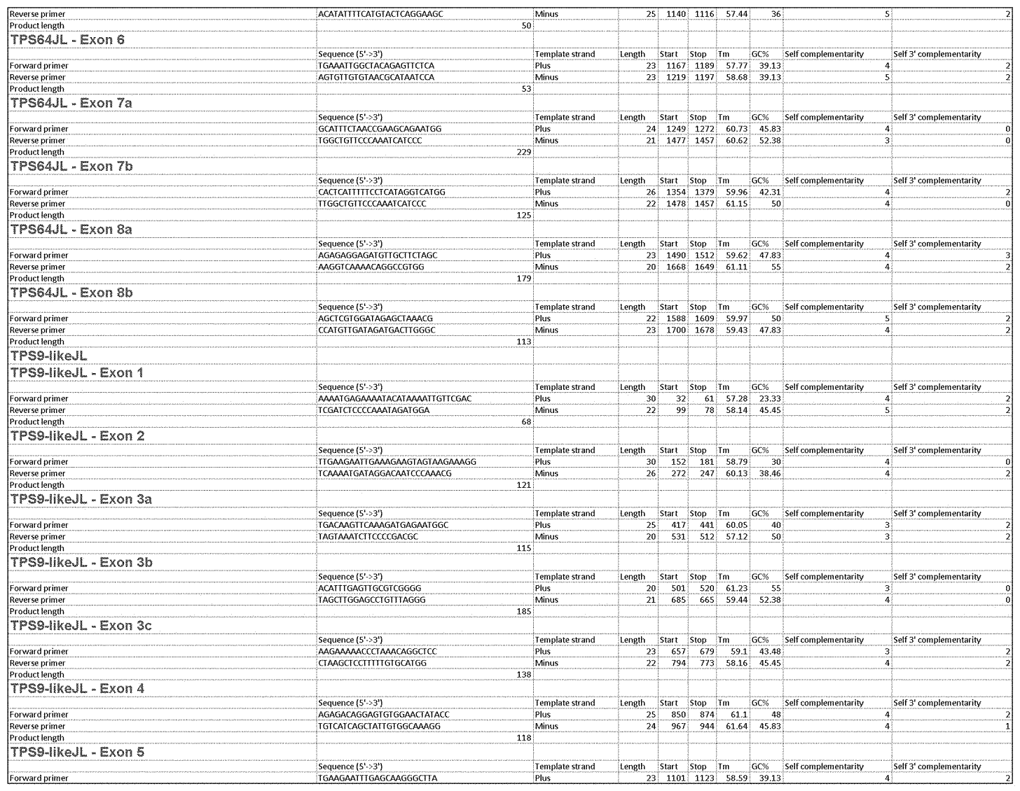

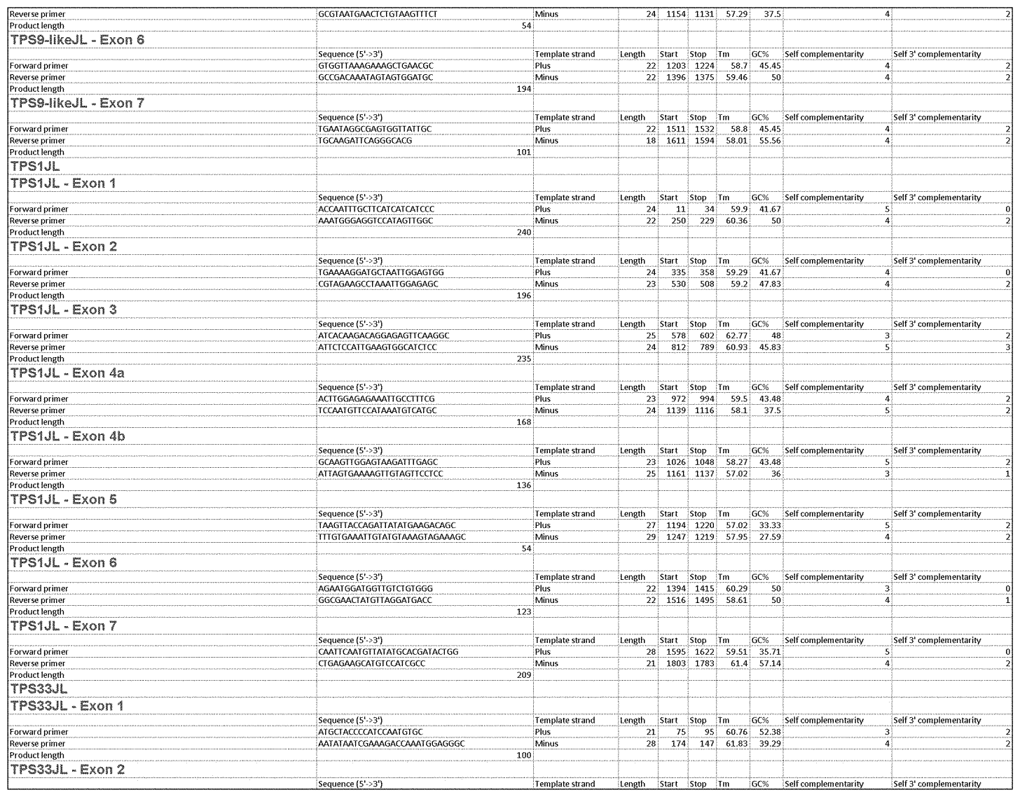

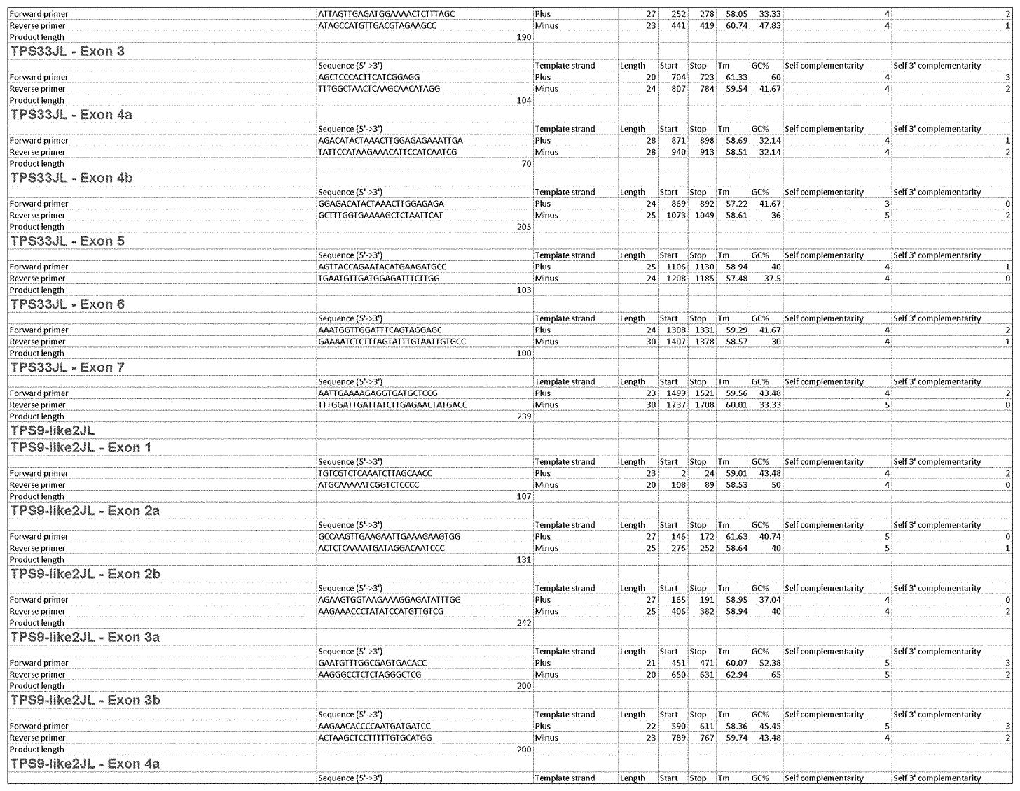

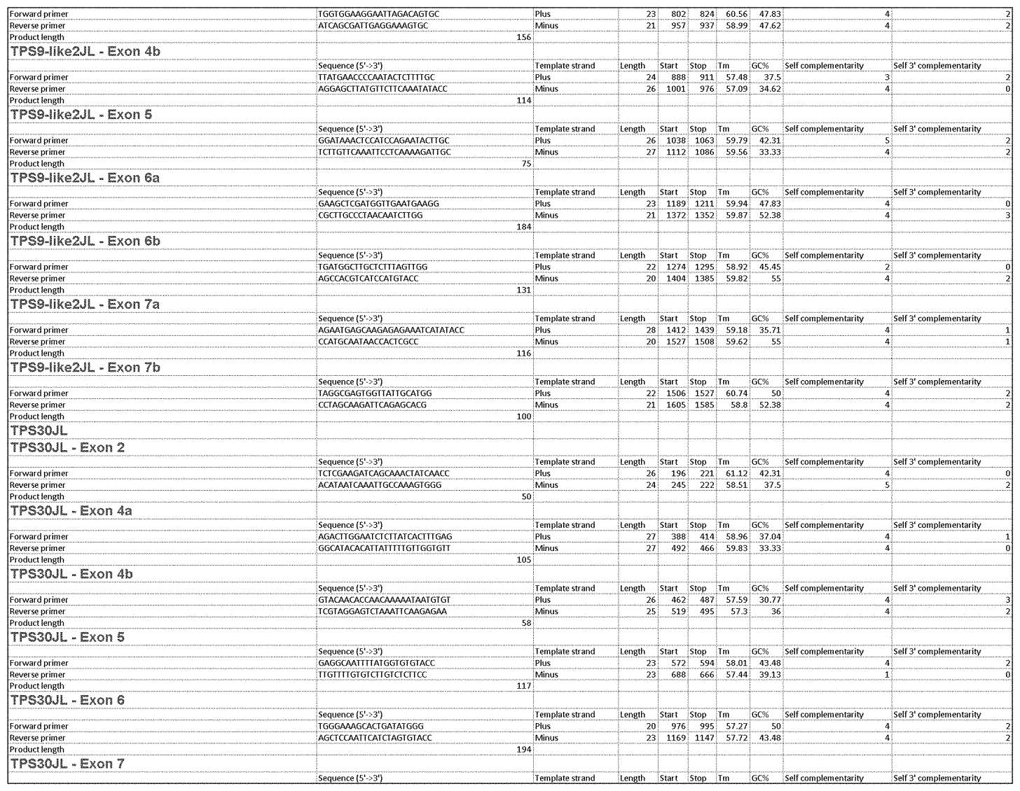

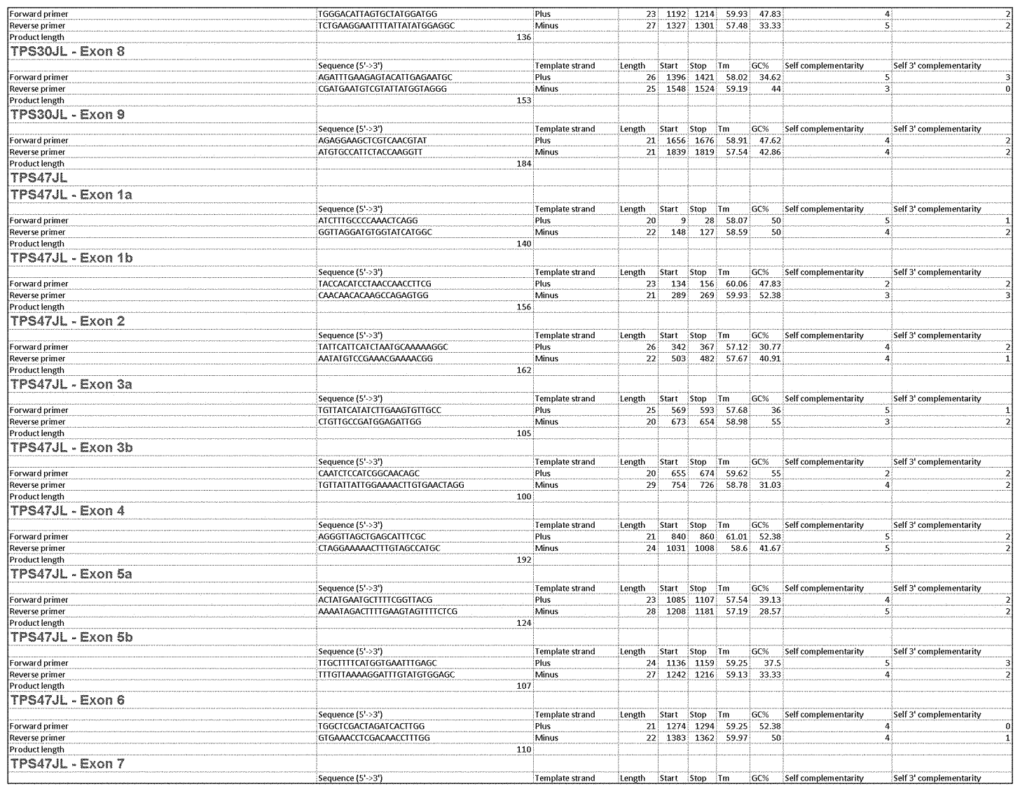

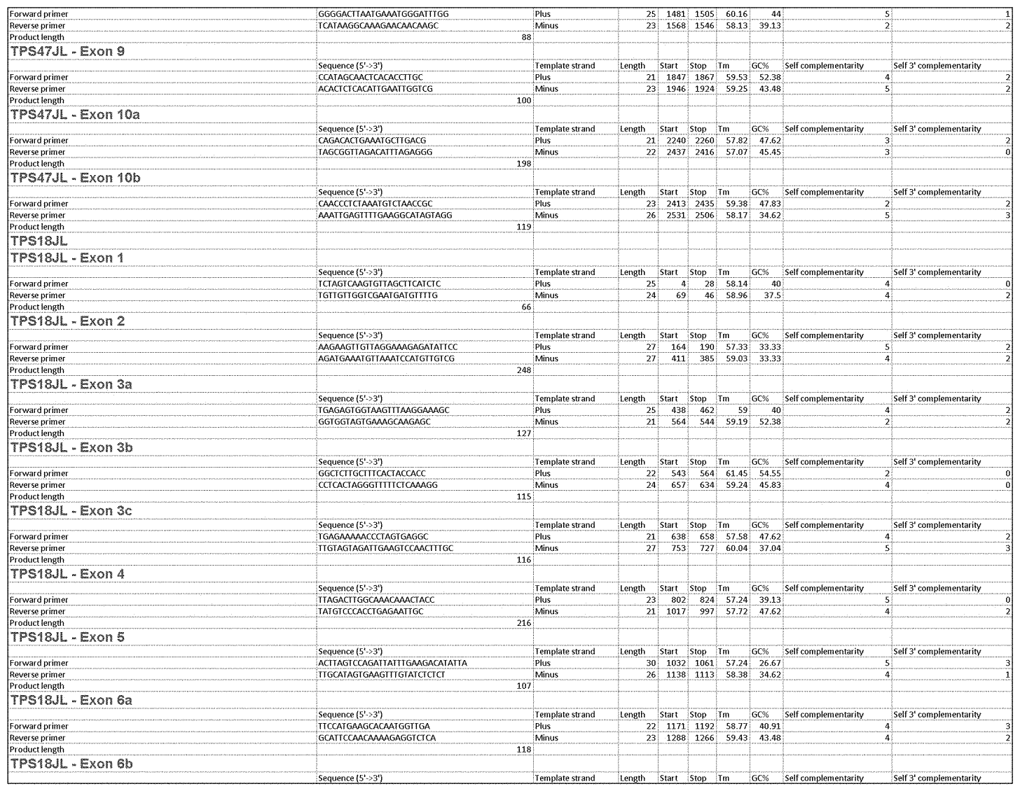

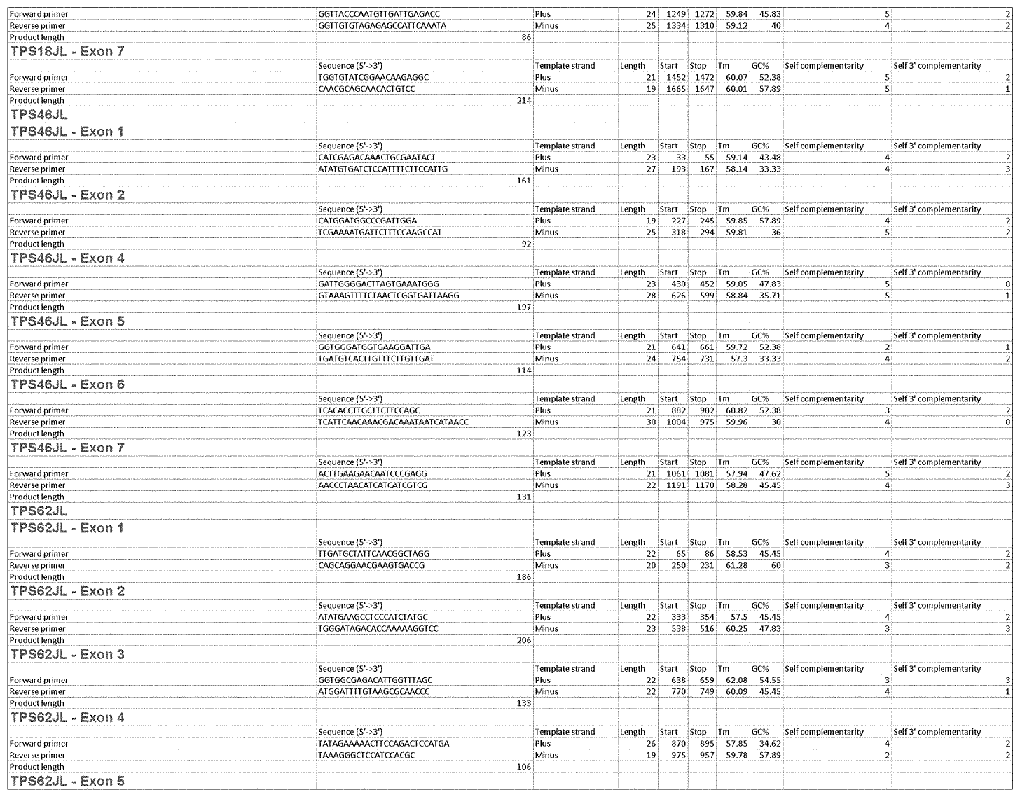

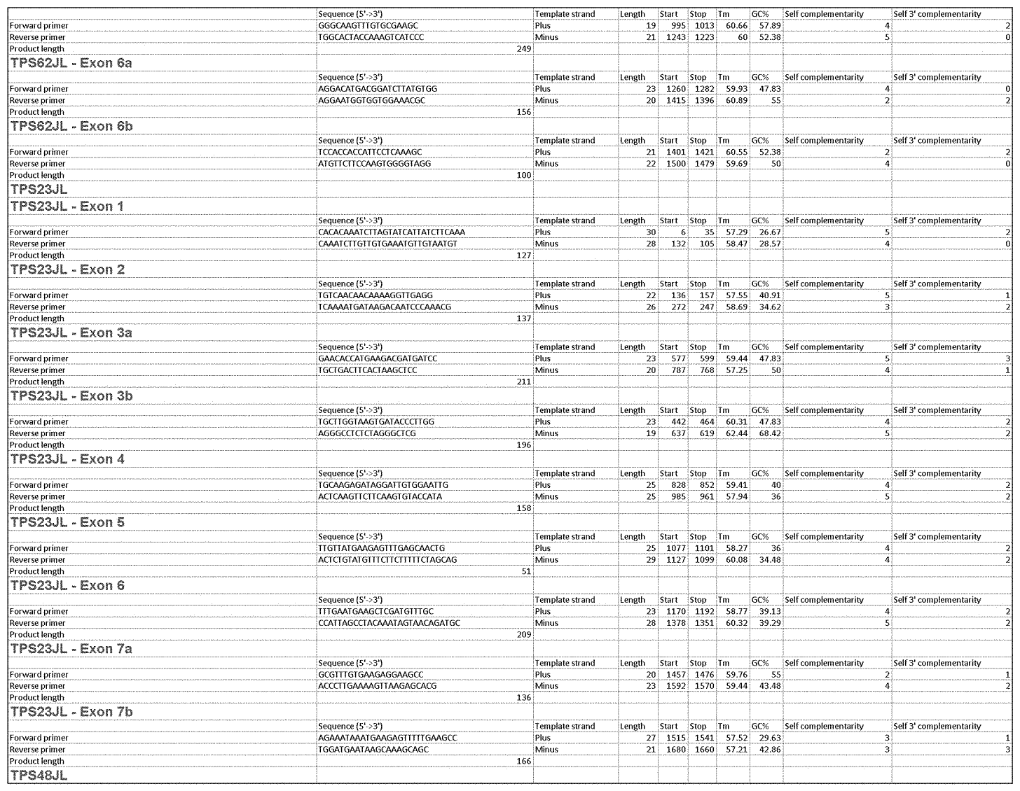

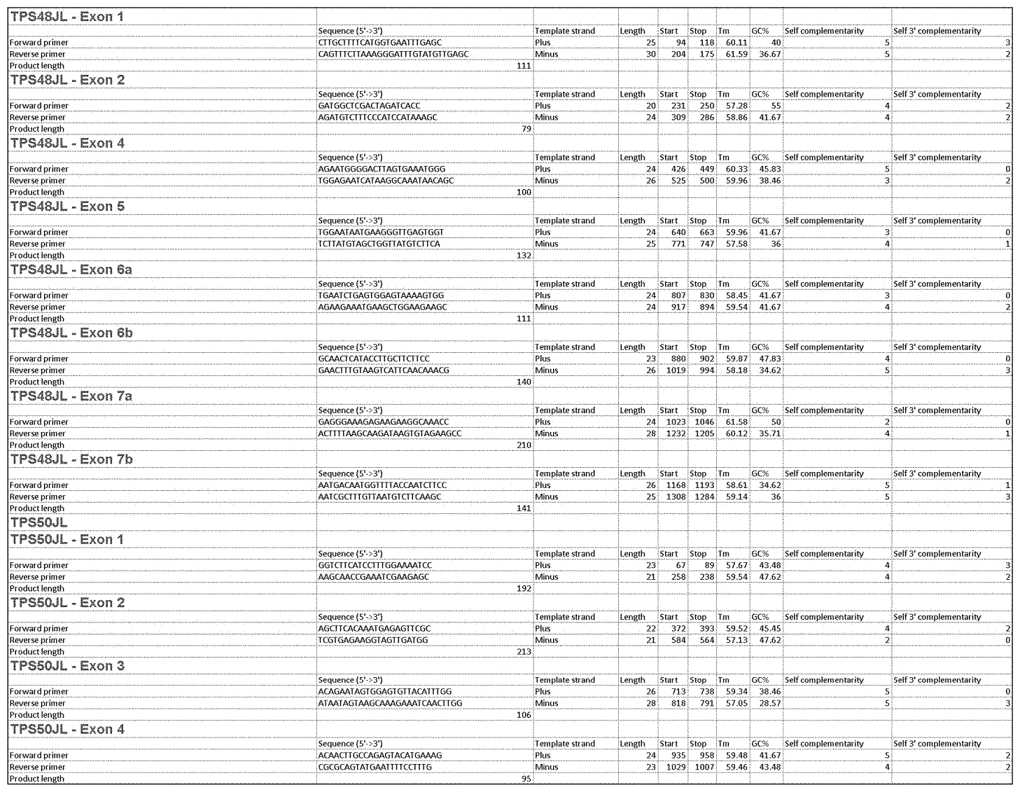

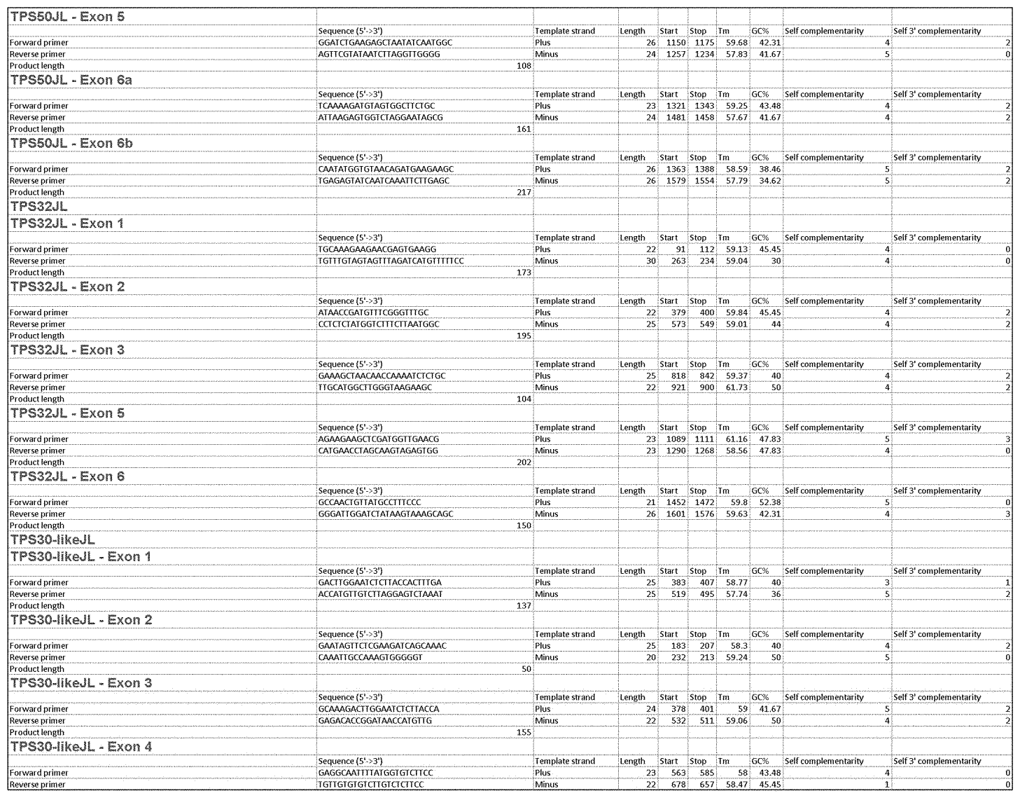

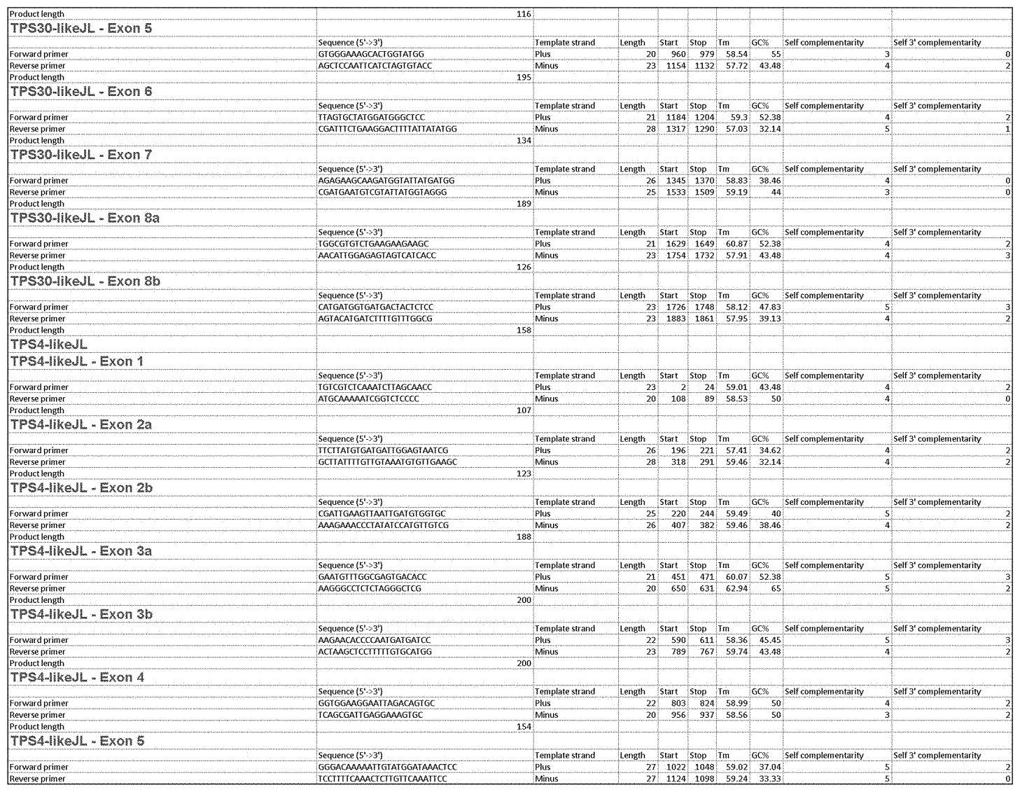

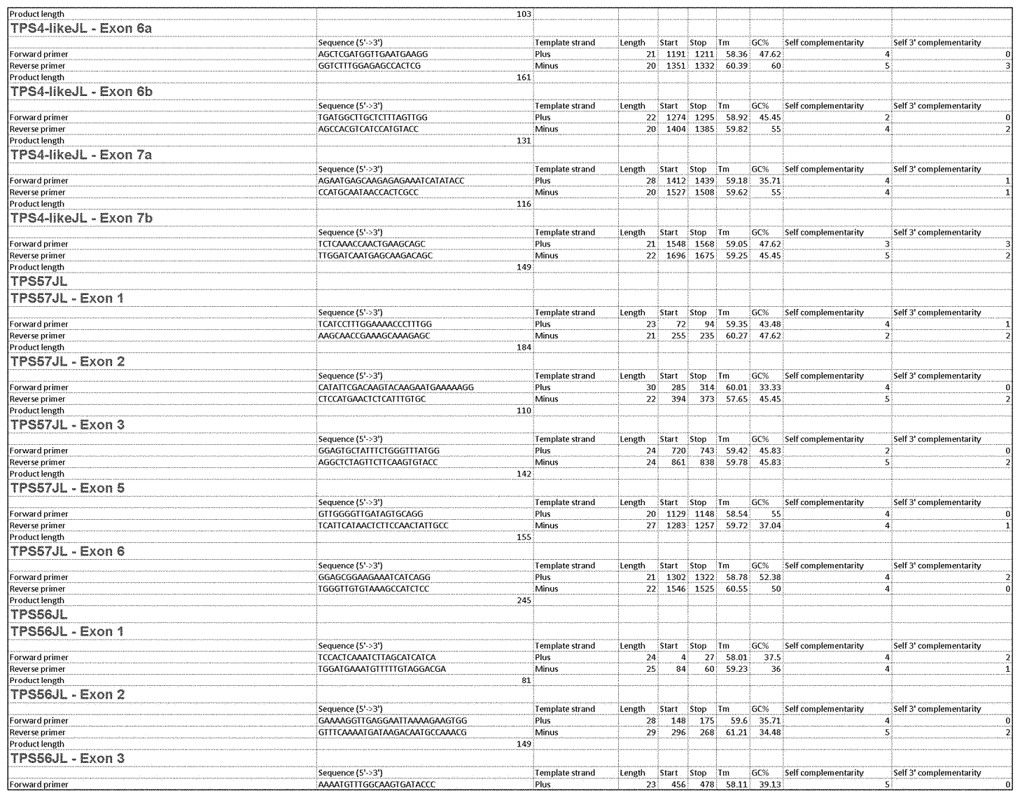

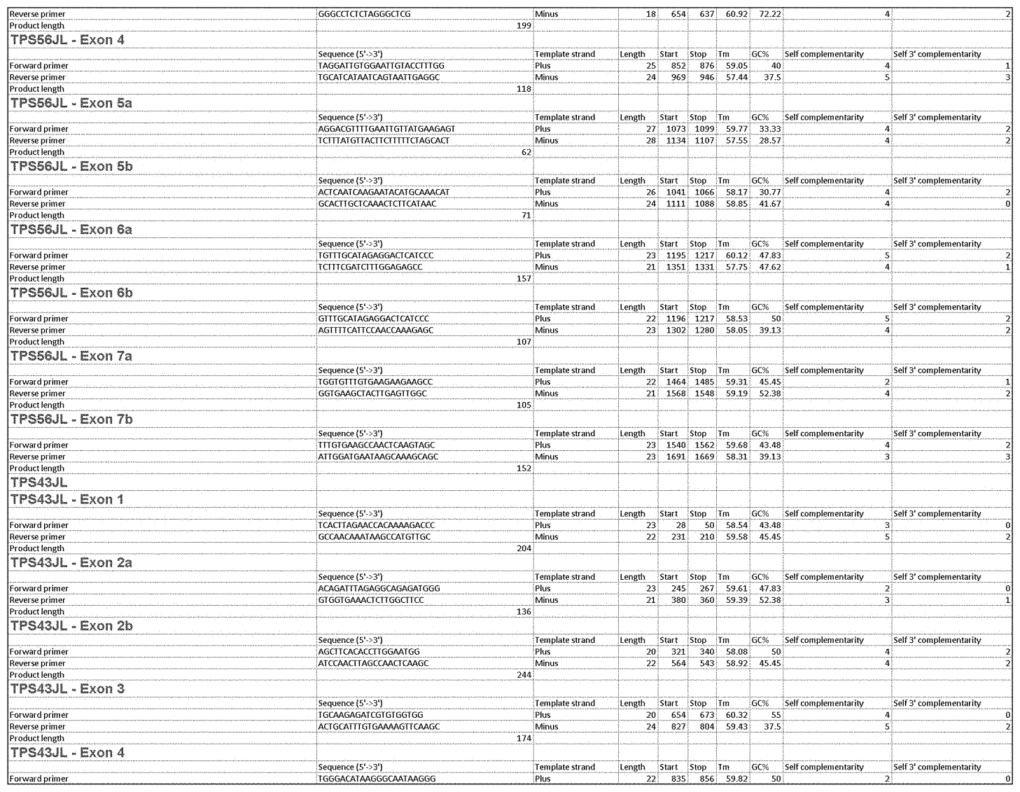

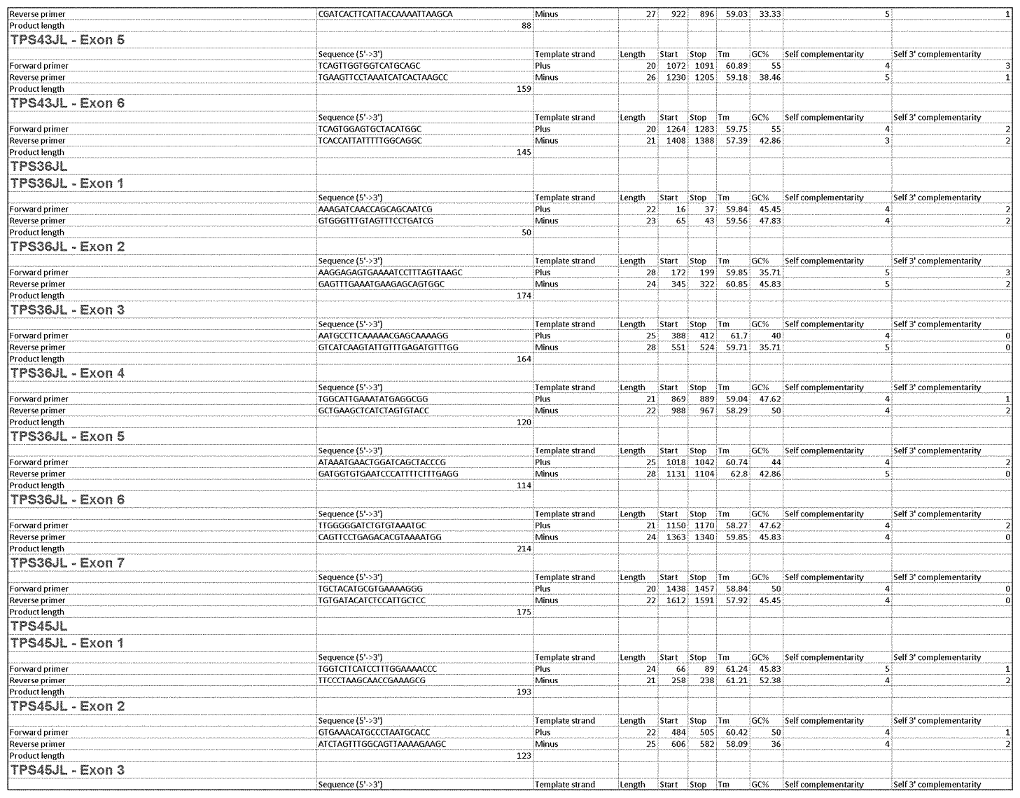

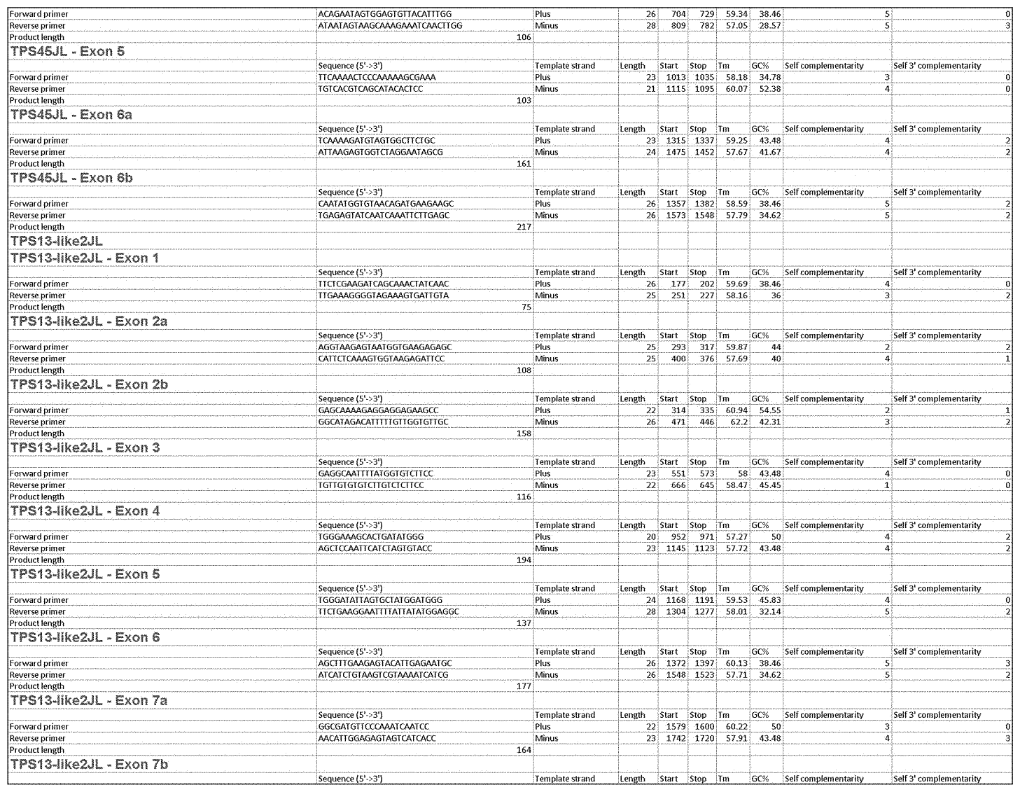

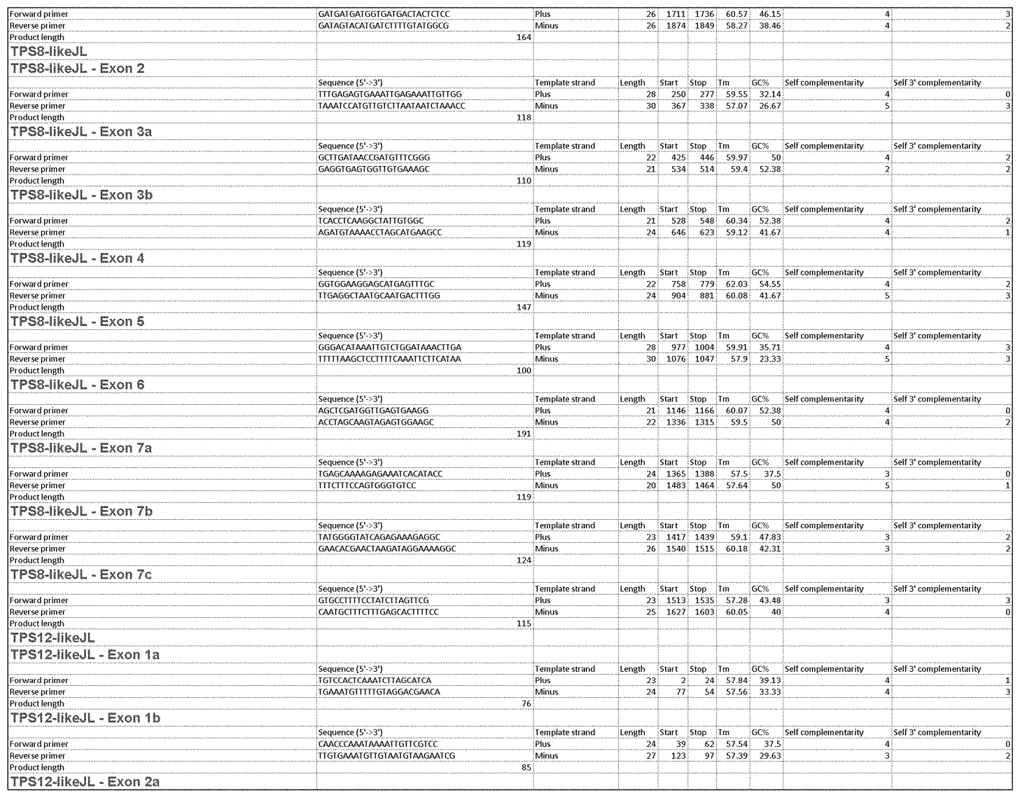

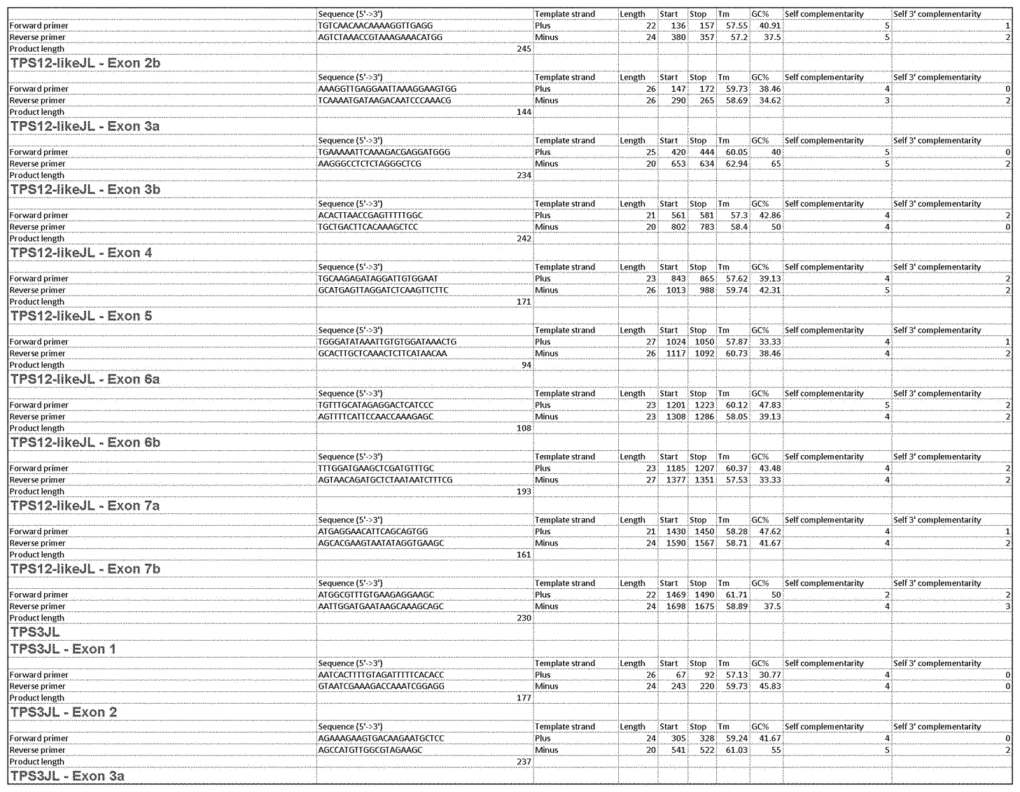

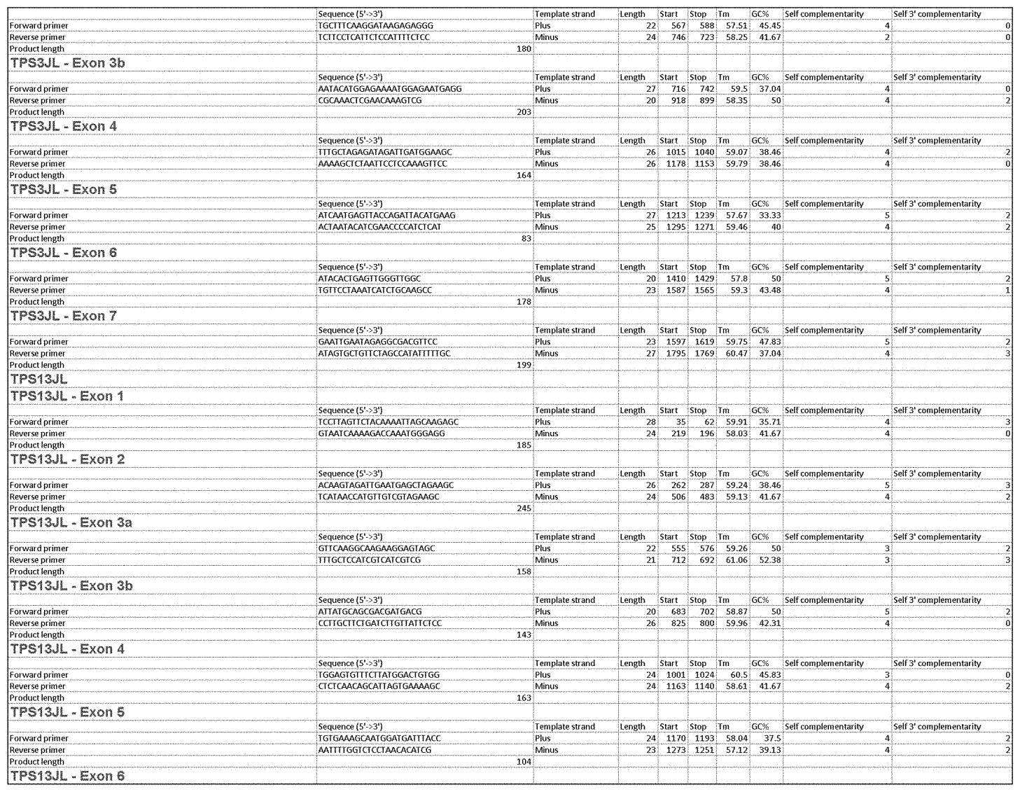

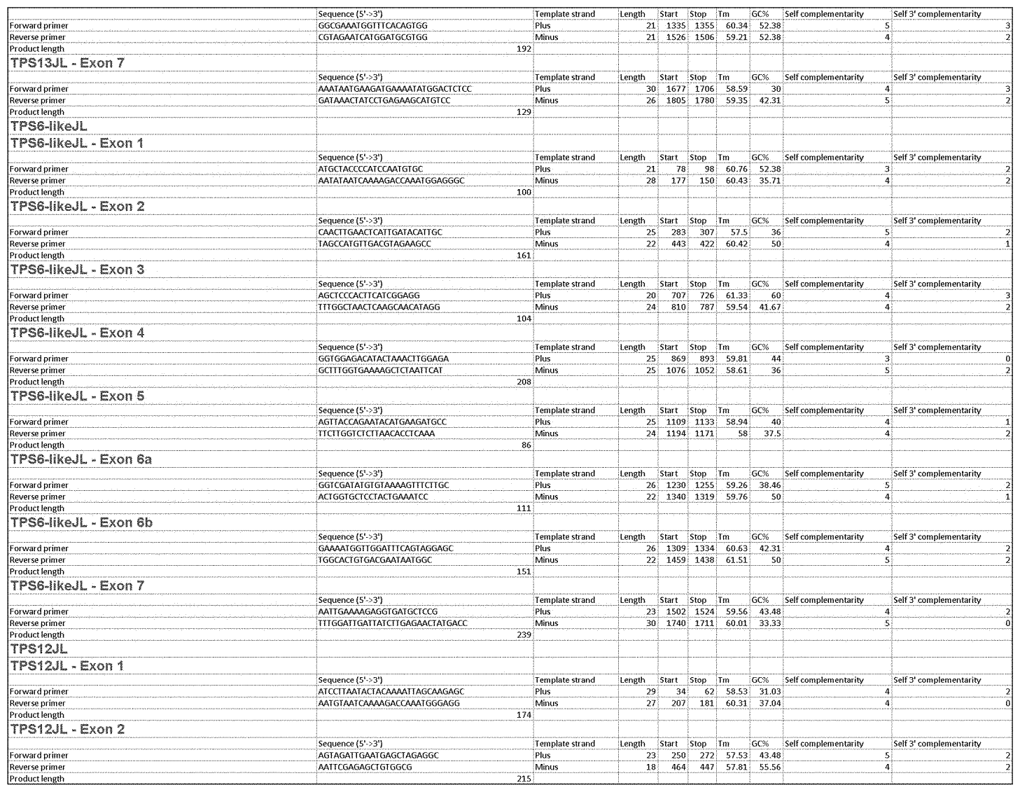

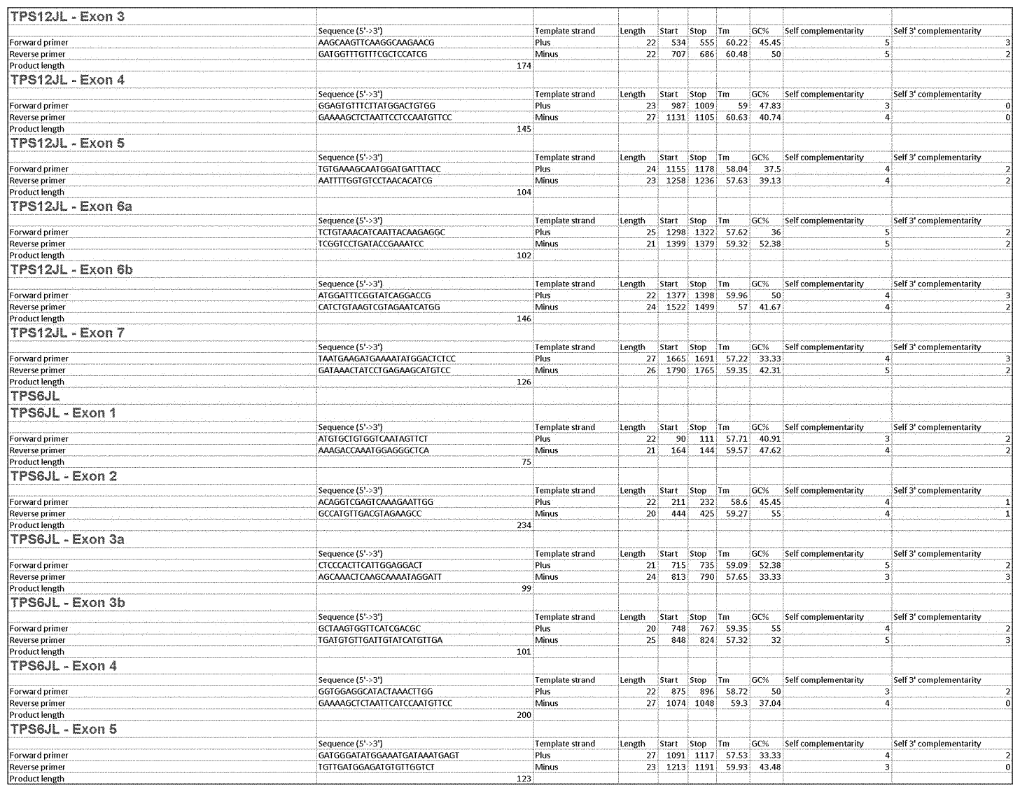

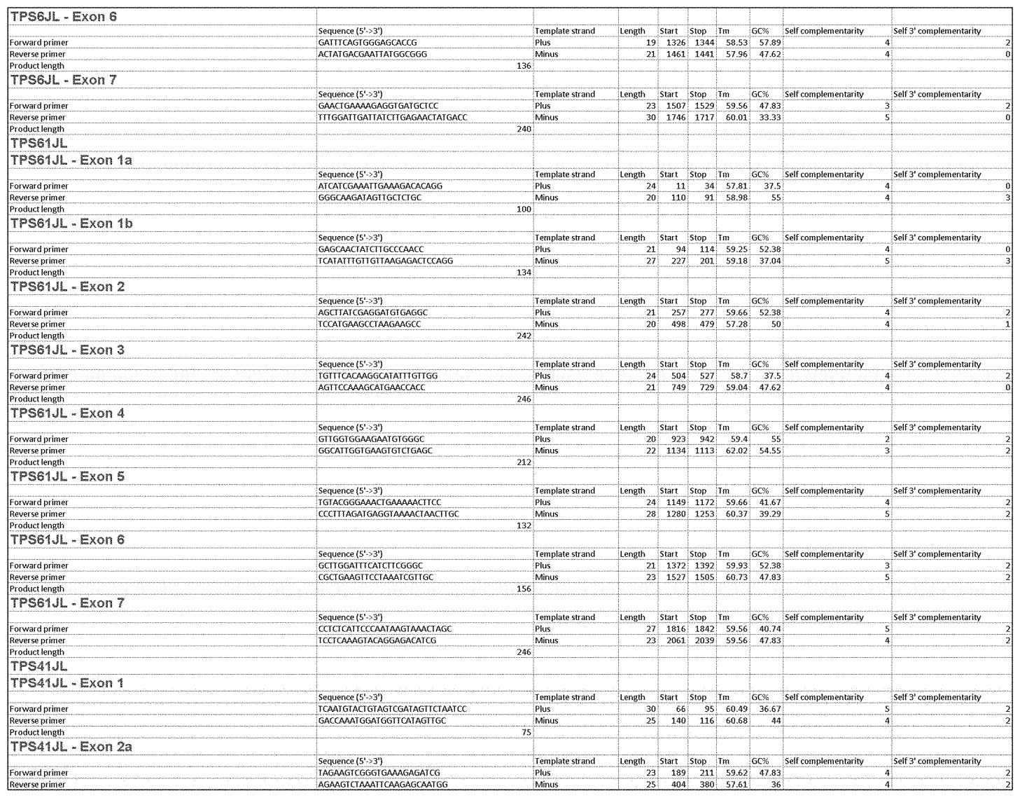

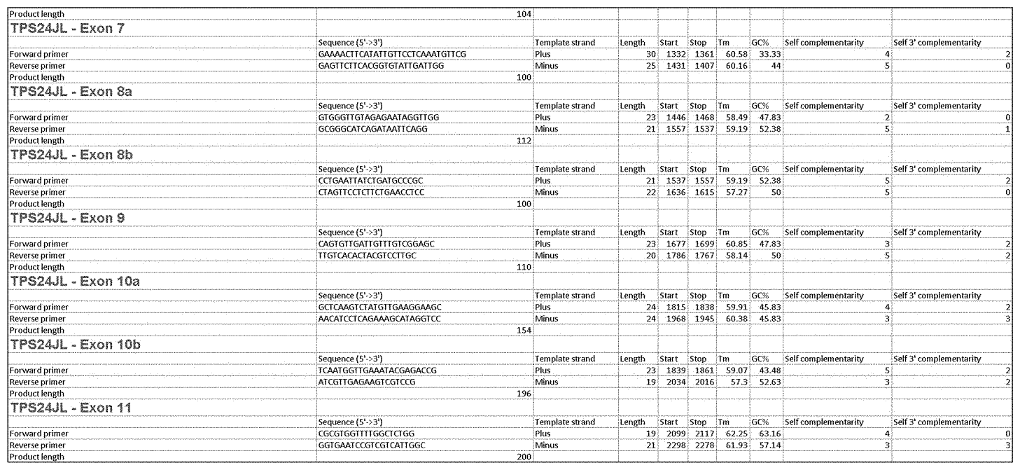

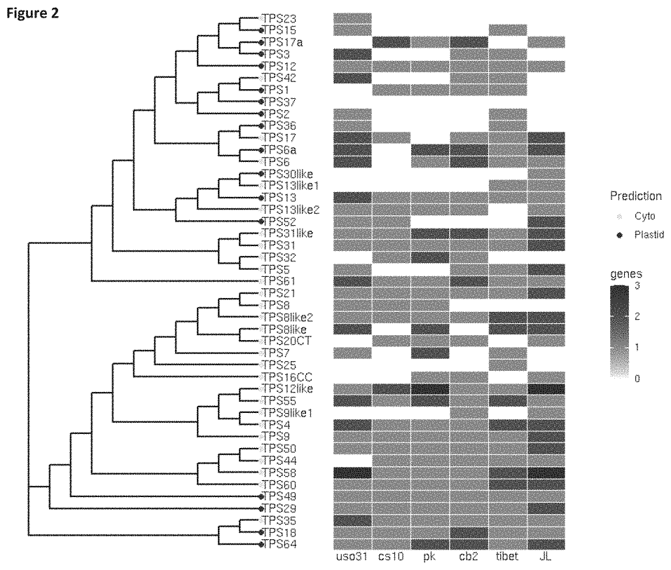

A reference set of all currently known TPS gene sequences was assembled, and this set was used as a query set for GenomeThreader, a gene structure prediction tool (version 1.7.3, Gremme et al., Inf. And Sotware Technol., 47(15):965-978 (2005)). GenomeThreader output for the six genomes was parsed to give a set of genomic segments, each paired with the most similar sequence from the reference set, along with a count of frameshifts. To further refine the gene structure and use additional data provided in the output files, each of these pairs was then searched with Exonerate (version 2.2, Slater et al., BMC Bioinformatics, 6(1):1-11 (2005)) run in exhaustive mode to get the optimal output for each alignment. Exonerate also was used to generate the cDNA sequence for each gene. The predicted cDNAs were compared to the reference data set using BLASTX (version 2.6.0, Altschul et al., Nucl. Acids Res., 25(17):3389-3402 (1997)). The BLAST output was evaluated for completeness and presence of in frame stop codons. Genes were assessed as complete if they had at least 7 exons (which is a well conserved standard for class a and b TPS genes), covered at least 95% of the query sequence length, and had no in frame stop codons. Statistics for the six genomes are shown in Table 6, and results of the gene discovery analysis are shown in the heat map (FIG. 2). For simplicity of analysis, genes were grouped into bins of 95% identity at the protein level. The heatmap shows the number of TPS genes (within 95% sequence identity) across six Cannabis genome assemblies. For each genome, white bands indicate no were genes found, light grey denotes 1 gene, medium grey denotes 2 genes, and black denotes 3 genes. To the left side of FIG. 2 is a phylogenetic tree of these genes, divided into 3 clades. The “TPS-b” clade, typically associated with monoterpene biosynthesis, is at the top, “TPS-a,” associated with sesquiterpene biosynthesis, is in the middle, and the small “TPS-g” clade, also associated with monoterpene biosynthesis, is at the bottom. Predicted plastid import signals (predicted using LOCALIZER, Sperschneider et al., Sci. Reports, 7(1): 1-14 (2017)), are shown as black dots. Light grey dots indicate no import signal was found.

The results show a remarkable amount of variation for TPS gene content across Cannabis varieties. A “core set” of genes (most of them sesquiterpene synthases) were found in every genome, although there may be multiple copies in any given genome. One of these is TPS9, the caryophyllene/humulene synthase, which can be expected given that beta-caryophyllene is the second most abundant terpene in Cannabis and is present at some level in the majority of strains. At the other end of the spectrum are what seem to be relatively rare genes that are only found in a single genome. One of these is a TPS that catalyzes the production of terpinolene, designated in some aspects as TPS37 (an example of which is the enzyme that catalyzes the production of terpinolene of Cannabis Sativa, csTPS37FN), the terpinolene synthase with expression in flower tissues. In previous work (Allen et al, PLoS ONE, 14(9): e0222363 (2019)), it was shown that terpinolene is an uncommon monoterpene present in just trace levels or below the detection limit in most strains of recreational Cannabis; in those strains where it is present, however, it typically is one of the dominant terpenes. This is consistent with presence/absence variation of the gene, and it is shown here.

Taken together, these results show that TPS gene content is a key differentiator between Cannabis strains. This means that the strains can be bred for specific terpene content, given a system for tracking the gene set in each parent, or in the offspring of a cross.

### Example 5

Design of Primers for Amplifying and Cloning Full-Length TPS Genes

Cloning primers were designed to amplify the following TPS genes 1) TPS9L4, 2) TPS9L21, 3) csTPS37FN cDNA (cleaved, no signal peptide sequence encoding the chloroplast import signal), 4) csTPS16CC, and 5) csTPS20CT. The primers were designed using full-length RNA sequence data. To create these targets, the primers were designed using a combination of by hand, primer3 analysis under standard/default conditions, and a Tm calculator for a specific range of 60° C. The primers (SEQ ID NOS:1330-1338) were screened by BLAST on the NCBI database and identified to be specific for amplifying full-length 1) TPS9L4 (LPA4 type), 2) TPS9L21 (LPA21.3 type), 3) csTPS37FN cDNA (cleaved, no signal peptide sequence encoding the chloroplast import signal), 4) csTPS16CC, and 5) csTPS20CT from the forward 5′ ATG start position bp position 1 and reverse 3′ TAA end minus 3bp position. For csTPS37FN cDNA, a unique forward primer was designed starting at bp position post chloroplast import signal sequence and an ATG was added to the 5′ end for reverse 3′ TAA minus 3bp position. To provide a bacterial vector insertion site in the target amplicon for molecular cloning, a 5′-end tag (5′-CACC-3′) was manually added to each forward primer after the primers were designed and checked computationally for specificity.

Mid-flower RNA (RNA from plant samples at 4-5 weeks post flower initiation) was extracted from Cannabis sativa samples (LPA 004, LPA 021.3 and LPA 005). The cDNA was synthesized and then subjected to a standard GoTaq PCR reaction (Promega, Madison, Wis.) using 5 primer pairs: SEQ ID NOS: 1330 and 1331; SEQ ID NOS: 1330 and 1332; SEQ ID NOS: 1333 and 1334; SEQ ID NOS: 1335 and 1336; and SEQ ID NOS: 1337 and 1338. Each of the primer sets were found to detect the presence or absence of specific Terpene synthases in Cannabis sativa, as seen by gel electrophoresis; Stringtie was used to identify the sequences corresponding to each of the terpene synthase genes. TPS9L21 (TPS9 LPA21.3 type) was detected in cDNA mid flower libraries of LPA4, LPAS, and LPA21.3. TPS9L4 (TPS9 LPA4) type, however, was detected only in LPA4 and LPAS mid flower cDNA libraries and not in LPA21.3. csTPS16CC was detected in cDNA mid flower libraries of LPAS and LPA21.3. csTPS20CT was detected in LPA4, LPAS, and LPA21.3 mid flower cDNA libraries. csTPS37FN was detected in cDNA mid flower libraries of LPAS and not in LPA4 or LPA21.3. To confirm their identity, all amplicons were excised from the gels and carried into downstream analysis of targeted sequencing via Oxford nanopore and molecular cloning and were followed up with functional characterization.

### Example 6

Analysis of gDNA and cDNA from Cannabis Plants by LAMP Assay

Total RNA, total gDNA, crude FTA extract (nucleic acid from extract from filter paper, such as Whatman, and synthesized cDNA templates were prepared from three distinct genotype/chemotypes of Type I Cannabis plants (de Meijer et al., Genetics, 163(1):335-346 (2003)). The samples were isolated from mid-flower tissue of the plants, and the samples for each of the distinct genotypes/chemotypes are named LPA4, LPAS, and LPA21.3.

For each of the samples, gDNA, cDNA and FTA crude extracts were subjected to GoTaq PCR reaction (Promega, Madison, Wis.) using primers B3 and F3 from csTPS37FN LAMP Primer Sets 1, 2 and 3 for the detection of csTPS37FN. Using the B3 and F3 primers from LAMP Primer Set 1 in a GoTaq PCR reaction using LPA005 gDNA, LPA005 cDNA, LPA004 gDNA, LPA004 cDNA, LPA021.3 gDNA, LPA021.3 gDNA, gel electrophoresis analysis showed that the target amplicons of interest are present after PCR as a non-specfic uniform sized ˜200bp amplicon product whether amplified from gDNA, cDNA, or FTA extract input in the LPA005 sample. The amplicon product was absent in LPA004 and LPA021.3 samples from either cDNA or gDNA.

The csTPS37FN B3/F3 Primer Set 1 target amplicons of interest were each excised from the gel, and the amplicons purified and sequenced by Sanger sequencing; 133 bp of DNA sequence was recovered with a 99.2% consensus agreement (132 bp out of 133 bp) between bands labeled TPS37-1 gDNA (LPA005 gDNA), TPS37-1 cDNA (LPA005 cDNA), and TPS37-1 FTA (LPA005 FTA Extract) samples and the csTPS37FN published reference sequences MK614216.1 and Finola (GCA_003417725.2). A single SNP (C to A) in the alignment was observed in the labeled TPS37-1 gDNA (LPA005 gDNA), TPS37-1 cDNA (LPA005 cDNA), and TPS37-1 FTA (LPA005 FTA Extract) samples relative to the csTPS37FN published reference sequences MK614216.1 and Finola (GCA_003417725.2).

Using the B3 and F3 primers from LAMP Primer Set 2 in a GoTaq PCR reaction on LPA005 gDNA, LPA005 cDNA, LPA004 gDNA, LPA004 cDNA, LPA021.3 gDNA, LPA021.3 gDNA LPA005 FTA Extract and NTC, as revealed by gel electrophoresis analysis, showed specific detection of distinct sized amplicons at ˜480 bp for LPA005 gDNA, at ˜200 bp for LPA005cDNA, and at ˜480 bp for LPA005 FTA extract. Amplicons were absent in LPA004 and LPA021.3 samples from either cDNA or gDNA input. The csTPS37FN B3/F3 Set 2 target amplicons of interest were each excised from the gel, the amplicons purified and sequenced by Sanger sequencing; 415 bp and 414 bp of DNA sequence was recovered from the LPA005 gDNA and LPA005 FTA extract with a 98.5% and 98.3% nucleotide consensus agreement (409 bp and 408 bp out of 415 bp) between the LPA005 gDNA and LPA005 FTA extract bands labeled TPS37-2 gDNA (LPA005 gDNA), TPS37-2 FTA extract (LPA005 FTA extract) and the csTPS37FN published reference sequence from Finola (GCA_003417725.2), while 141bp of DNA sequence was recovered from the LPA005 cDNA with a 100% nucleotide similarity consensus agreement (141 bp out of 141 bp) between the LPA005 cDNA bands labeled TPS37-2 cDNA (LPA005 cDNA) and the published reference sequence from Finola (GCA_003417725.2).

Using the B3 and F3 primers from LAMP Primer Set 3, in a GoTaq PCR reaction on LPA005 gDNA, LPA005 cDNA, LPA004 gDNA, LPA004 cDNA, LPA021.3 gDNA, LPA021.3 gDNA LPA005 FTA Extract, and NTC, gel electrophoresis analysis showed specific detection of distinct sized amplicons at ˜500 bp for LPA005 gDNA, at ˜250 for LPA005cDNA, and at ˜500 bp for the LPA005 FTA extract. The target amplicons of interest are present after PCR whether amplified from gDNA, cDNA, or FTA extract from in the LPA005 sample. Amplicons were absent in LPA004 and LPA021.3 samples from either cDNA or gDNA input. The csTPS37FN B3/F3 Set 3 target amplicons of interest were each excised from the gel, the amplicons purified and sequenced by Sanger sequencing; 437 bp and 427 bp of DNA sequence was recovered from the LPA005 gDNA and LPA005 FTA extract with a 98.8% and 96.5% nucleotide similarity consensus agreement (432 bp and 422 bp out of 437 bp) between the LPA005 gDNA and LPA005 FTA extract bands labeled TPS37-3 gDNA (LPA005 gDNA), TPS37-3 FTA extract (LPA005 FTA extract) and the csTPS37FN published reference sequence from Finola (GCA_003417725.2), while 180 bp of DNA sequence was recovered from the LPA005 cDNA with a 100% nucleotide similarity consensus agreement (180 bp out of 180 bp) between the LPA005 cDNA bands labeled TPS37-2 cDNA (LPA005 cDNA) and the published reference sequence from Finola (GCA_003417725.2).

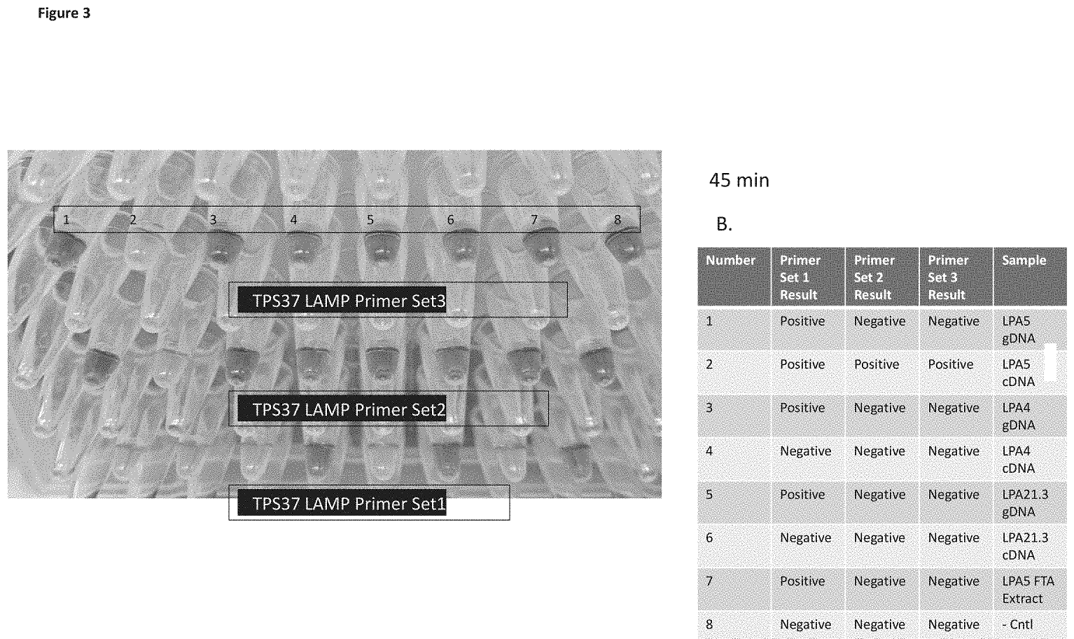

Using csTPS37FN LAMP Primer Sets 1, 2, and 3, 1 uL of the following input from sample numbers 1) LPA005 gDNA, 2) LPA005, 3) LPA004 gDNA, 4) LPA004 cDNA, 5) LPA021.3 gDNA, 6) LPA021.3 cDNA, 7) LPA005 FTA extract, and 8) NTC were analyzed. The sample set was loaded into a csTPS37FN LAMP based assay detection reaction prepared with NEB WarmStart Colorimetric LAMP Mastermix Mix (New England Biolabs, Ipswich, MA), csTPS37 LAMP Primer sets 1, 2, or 3, and nuclease free water. At time 0, all LAMP reactions are seen as dark grey. After a 45 minute reaction at 65° C., positive LAMP reactions are seen as a pale liquid and negative LAMP reactions are seen as dark grey (FIG. 3). LPA005 gDNA, LPA005 cDNA, LPA004 gDNA, LPA021.3 gDNA, and LPA005 FTA extract are seen as a pale liquid in the LAMP Primer Set 1 reactions and recorded positive; LPA004 cDNA, LPA021.3 cDNA and the negative control are seen as dark grey in the LAMP Primer Set 1 reactions and recorded negative. LPA005 cDNA are seen as pale grey in LAMP Primer Sets 2 and 3 reactions and are recorded positive; LPA005 gDNA, LPA004 gDNA, LPA004 cDNA, LPA021.3 gDNA, LPA004 cDNA, and FTA extract are seen as dark grey in LAMP Primer Sets 2 and 3 reactions and recorded negative. The results demonstrate specific detection of expressed csTPS37FN in LPAS cDNA using csTPS37FN LAMP Primer Sets 2 and 3. Non-specific detection using csTPS37FN LAMP Primer set 1 was observed in cDNA and FTA extract for LPA005 and gDNA for LPA004, LPA005, and LPA021.3. Positive detection of accumulated amplicon is seen as pale samples rather than dark grey samples.

### Example 7

Functional Characterization of Bacterially Expressed TPS37

An in-vitro functional characterization assay of bacterially expressed csTPS37FN cloned from LPA005 was performed. A TPS Assay Buffer was prepared fresh with a final formulation of 25mM HEPES, 100 mM KC1, 10 mM MgCl₂, 5% Glycerol, and 5 mM DTT (Booth 2017, see reference as described for FIG. 4). Geranyl Pyrophosphate (GPP) and Farnesyl pyrophosphate (FPP) (Isoprenoids.com, Tampa Fla.) were used as substrates for the in vitro assay. The test samples included TPS assay buffer only, TPS assay buffer +16 uM GPP, TPS assay buffer +13uM FPP, TPS assay buffer +csTPS37FN (5 μg) +16 uM GPP, and TPS assay buffer +csTPS37FN +16 uM FPP, and 1 ppm terpinolene standard diluted in pentane (Sigma-aldrich, St. Louis, Mo.). The in-vitro assay tested purified protein that was prepared for functional activity studies, as demonstrated by GC-MS detection of product terpenes. The test samples included buffer only, buffer+GPP, buffer+FPP, buffer+csTPS37FN+GPP, and buffer+csTPS37FN+FPP, and a terpinolene standard. The test samples were prepared in a 500 uL reaction volume with buffer and substrate, overlaid with pentane, incubated at 35° C. for 4 hours and then assayed by a full evaporation headspace method and GC-MS. The presence or absence of 44 terpene compounds were measured. The results showed that in the buffer+csTPS37FN+GPP reaction, functional activity is observed in the form of production of terpinolene; the data demonstrated a signal to noise (S/N) of 73.63 to 1 and area of 1307 for terpinolene production and lesser amounts of 3-carene, alpha- and betα-pinene. All measurements of detected compounds in the buffer+csTPS37FN+GPP reaction were below limits of quantitation for this instrument; however, positive detection is confirmed by the presence of a quantitation ion m/z and reference ions m/z for terpinolene and quantitation ion m/z for 3-carene and alpha and beta pinenes, respectively. Observation of presence of a quantitation ion m/z and reference ions m/z for terpinolene in the terpinolene standard was also confirmed. No terpenes were observed in the test buffer only, buffer+GPP, buffer+FPP, and buffer+csTPS37FN+FPP reactions (csTPS37FN only accepts GPP and not FPP as a precursor for terpene synthesis).

### Example 8

Examples of Embodiments

A1. A method of analyzing a plant cultivar comprising at least one terpene synthase gene or a 20paralog thereof, the method comprising:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique subsequence of the terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene or a paralog thereof is different than the subsequences of other terpene synthase genes and/or paralogs thereof;
(c) amplifying the mixture, thereby obtaining an amplified mixture; and
(d) analyzing the amplified mixture of (c), whereby at least one terpene synthase gene or a paralog thereof is identified and/or quantified in the amplified mixture.

A1.1. A method of preparing nucleic acid comprising at least one terpene synthase gene or a paralog thereof from a plant cultivar comprising the at least one terpene synthase gene or a paralog thereof, the method comprising:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique subsequence of the terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene is different than the subsequences of the other terpene synthase genes and/or paralogs thereof;
(c) amplifying the mixture, thereby obtaining an amplified mixture; and
(d) analyzing the amplified mixture of (c), whereby at least one terpene synthase gene or a paralog thereof is identified and/or quantified in the amplified mixture and the amplified mixture is identified as comprising prepared nucleic acid comprising at least one terpene synthase gene or a paralog thereof from the plant cultivar.

A2. The method of embodiment A1 or A1.1, wherein the plant cultivar comprises a plurality of terpene synthase genes and/or paralogs thereof and the method comprises:
in (b), contacting the nucleic acid sample with a plurality of polynucleotide primer pairs under amplification conditions, thereby preparing a mixture, wherein each of the plurality of polynucleotide primer pairs hybridizes to a unique subsequence of a terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene or a paralog thereof is different than the subsequences of the other terpene synthase genes and/or paralogs thereof; and
in (d), a plurality of terpene synthase genes and/or paralogs thereof are identified and/or quantified.

A3. The method of any one of embodiments A1, A1.1 or A2, wherein each of the primers of a polynucleotide primer pair hybridizes to a conserved region of the subsequence and the hybridized polynucleotide primer pair flanks a variable region of the subsequence.

A4. The method of any one of embodiments A1 to A3, wherein the subsequence is an exon, an intron, a portion within an exon or a portion within an intron.

A5. The method of any one of embodiments A1 to A4, wherein the subsequence is an exon or a portion within an exon.

A6. The method of any one of embodiments A1 to A5, wherein the identification in (d) is by one or more of high-resolution melting (HRM), quantitative PCR (qPCR), loop-mediated isothermal amplification (LAMP), restriction endonuclease digestion, gel electrophoresis and sequencing.

A7. The method of any one of embodiments Ato A6, wherein one or more of the polynucleotide primer pairs are selected from among those set forth in SEQ ID NOS: 1-1284.

A7.1. The method of any one of embodiments A1 to A7, wherein one or more of the polynucleotide primer pairs are selected from among those set forth in SEQ ID NOS: 1-1284, or from among sequences that share 90%, 91%, 92%, 93%, 94%, 95%, 96%, 97%, 98%, 99% or more identity with any of the sequences set forth in SEQ ID NOS: 1-1284.

A7.2. The method of any one of embodiments A1 to A7.1, wherein at least one terpene synthase is a paralog of a terpene synthase gene.

A7.3. The method of any one of embodiments A1 to A5 and A7.2, wherein the identification and/or quantification in (d) is by loop-mediated isothermal amplification (LAMP).

A7.4. The method of embodiment A7.3, wherein a TPS37 gene is identified and/or quantified in (d)

A7.5. The method of embodiment A7.4, wherein the polynucleotide primer pairs are present in a set of primers selected from among SEQ ID NOS:1285-1293, SEQ ID NOS:1294-1302, SEQ ID NOS:1303-1311, SEQ ID NOS:1312-1319 and SEQ ID NOS:1320-1327.

A8. The method of any one of embodiments A1 to A7.5, wherein the terpene synthase genes and/or paralogs thereof that are identified and/or quantified are monoterpene synthase genes and/or paralogs thereof, diterpene synthase genes and/or paralogs thereof, sesquiterpene synthase genes and/or paralogs thereof or any combination thereof.

A9. The method of any one of embodiments A1 to A8, wherein, based on the terpene synthase genes and/or paralogs thereof that are identified and/or quantified, the terpene synthase gene and/or paralog expression profile and/or the terpene production profile of the plant cultivar is determined.

A10. The method of embodiment A9, wherein the terpene synthase gene and/or paralog expression profile and/or the terpene production profile is of the root, flower, stem, leaf or any combination thereof.

A11. The method of any one of embodiments A1 to A10, wherein, based on the terpene synthase genes and/or paralogs thereof that are identified and/or quantified and/or based on the terpene synthase gene expression profile that is determined and/or based on the terpene production profile that is determined, a lineage of the plant cultivar is assigned.

A11.1. The method of any one of embodiments A1 to A11, wherein, based on identifying and/or quantifying at least one terpene synthase gene or a paralog thereof in (d), determining the terpene synthase gene profile, the terpene synthase expression profile, the terpene production profile, the cannabinoid production profile, the flavonoid production profile, or any combination thereof in the plant cultivar.

A12. The method of any one of embodiments A1 to A11.1, wherein, based on the terpene synthase genes and/or paralogs thereof that are identified and/or quantified and/or based on the terpene synthase gene and/or paralog thereof expression profile that is determined and/or based on the terpene production profile that is determined, a medicinal use of the plant cultivar is assigned.

A13. The method of any one of embodiments A1 to A12, wherein, based on the terpene synthase genes and/or paralogs thereof that are identified and/or quantified and/or based on the terpene synthase gene and/or paralog thereof expression profile that is determined and/or based on the terpene production profile that is determined, that is determined, the plant cultivar is identified as resistant to an organism or situation.

A14. The method of embodiment A13, wherein the organism or situation is selected from among insects, pests, mold, chemicals, mildew, fungi, bacteria, an environmental condition or a geographic location.

A15. The method of any one of embodiments A1 to A12, wherein, based on the terpene synthase genes and/or paralogs thereof that are identified and/or quantified and/or based on the terpene synthase gene and/or paralog thereof expression profile that is determined and/or based on the terpene production profile that is determined, the plant cultivar is identified as having affinity towards an organism or situation.

A16. The method of embodiment A15, wherein the organism or situation is selected from among insects, pests, mold, mildew, fungi, bacteria, an environmental condition or a geographic location.

A17. The method of any one of embodiments A1 to A16, wherein a plurality of plant cultivars are analyzed.

A18. The method of embodiment A17, wherein the plant cultivars are of the same species.

A19. The method of embodiment A17 or A18, comprising classifying the plurality of plant cultivars based on lineage.

A20. The method of any one of embodiments A17 to A19, comprising classifying the plurality of plant cultivars based on medicinal use.

A20.1. The method of any one of embodiments A1 to A20, wherein one or more plant cultivars is/are of the family Rosidae.

A21. The method of any one of embodiments A1 to A20, wherein one or more plant cultivars is/are a Cannabis cultivar.

A21.1. The method of embodiment A21, whereib the Cannabis cultivar is selected from among one or more of Type 1, Type 2, Type 3, Type 4 and Type 5 cultivars

A22. The method of embodiment A21, wherein the monoterpene synthase genes and/or paralogs thereof of the Cannabis plant cultivar are identified and/or quantified and, based on the identified and/or quantified monoterpene synthase genes and/or the expression profile of the identified and/or quantified monoterpene synthase genes and/or paralogs thereof, the terpene production profile, the cannabinoid production profile, the flavonoid production profile, or any combination of two or more of the terpene production profile, the cannabinoid production profile and the flavonoid production profile of the Cannabis plant cultivar is determined.

A23. The method of embodiment A22, wherein, based on the cannabinoid production profile, the flavonoid production profile, or the cannabinoid production profile and the flavonoid production profile that is determined, a lineage of the Cannabis plant cultivar is assigned.

A24. The method of embodiment A22 or A23, wherein, based on the cannabinoid production profile, the flavonoid production profile, or the cannabinoid production profile and the flavonoid production profile that is determined, a medicinal use of the Cannabis plant cultivar is assigned.

A25. The method of any one of embodiments A21 to A24, wherein a plurality of Cannabis plant cultivars are analyzed.

A26. The method of embodiment A25, comprising classifying the plurality of Cannabis plant cultivars based on lineage.

A27. The method of embodiment A25, comprising classifying the plurality of plant cultivars based on medicinal use.

A28. The method of any one of embodiments A1 to A27, wherein at least one plant cultivar that is analyzed produces one or more terpenes selected from among α-Bisabolol, endo-Borneol,

Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

A29. The method of any one of embodiments A9 to A27, wherein a terpene production profile is determined for one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

A30. The method of any one of embodiments A1 to A29, wherein at least one plant cultivar that is analyzed expresses one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPSS, TPSS, TPS60, TPS61, TPS62, TPS63, TPS64, TPS6, TPS6-like, TPS7, TPS8, TPS8, TPS8-like, TPS9, TPS9, TPS9-like and TPS9-like2.

A30.1 The method of any one of embodiments A1 to A30, wherein at least one plant cultivar that is analyzed expresses one or more terpene synthases selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

A31. The method of any one of embodiments A9 to A29, wherein a terpene synthase expression profile is determined for one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPSS, TPSS, TPS60, TPS61, TPS62, TPS63, TPS64, TPS6, TPS6-like, TPS7, TPS8, TPS8, TPS8-like, TPS9, TPS9, TPS9-like and TPS9-like2.

A31.0. The method of any one of embodiments A9 to A31, wherein at least one plant cultivar that is analyzed expresses one or more terpene synthases selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

A31.1. The method of any one of embodiments A28 to A31.0, wherein one or more plant cultivars is/are of the family Rosidae.

A31.2. The method of any one of embodiments A1 to A31.1, wherein one or more plant cultivars is/are a Cannabis cultivar.

A31.3. The method of embodiment A31.2, whereib the Cannabis cultivar is selected from among one or more of Type 1, Type 2, Type 3, Type 4, and Type 5 cultivars

A31.4. The method of any one of embodiments A28 to A31.3, wherein the plant cultivar is a Cannabis cultivar selected from among Jamaican Lion, Purple Kush, CannaTsu, Finola, Valley Fire and Cherry Chem.

A32. The method of any one of embodiments A1 to A31.4, further comprising, based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for in-breeding or out-crossing.

A33. The method of embodiment A32, wherein the plant cultivar is selected for its lineage that is assigned based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof.

A34. The method of embodiment A32 or A33, wherein the plant cultivar is selected for a medicinal use that is assigned based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof.

A34.1. The method of embodiment A34, wherein the plant cultivar is selected for a medicinal use that is assigned based on identifying one or more terpene synthase genes, determining the expression profile of one or more terpene synthase genes, and/or determining the production profile of one or more terpenes.

A34.2. The method of embodiment A34 or A34.1, wherein the medicinal use is selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

A34.3. The method of embodiment A34 or A34.1, wherein the medicinal use is to impart energy, mental clarity, appetite stimulation or appetite suppression.

A34.4. The method of any one of embodiments A34 to A34.3, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting one or more medicinal uses to a plant cultivar.

A34.5. The method of any one of embodiments A34 to A34.4, wherein the one or more TPS genes, or sets thereof, produce one or more of the terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

A34.6. The method of any one of embodiments A34 to A34.5 that is a method of breeding for one or more offspring cultivars that show increased cannabinoid production compared to at least one of the parent cultivars.

A34.7. The method of embodiment A34.6, wherein the one or more offspring cultivars show reduced expression, or lack of expression, of one or more terpene synthases selected from among TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL and TPS37JL compared to at least one of the parent cultivars.

A34.8. The method of any one of embodiments A34 to A34.7 that is a method of breeding for one or more offspring cultivars that produces an increased energetic effect compared to at least one of the parent cultivars.

A34.9. The method of embodiment A34.8, wherein the one or more offspring cultivars comprises a terpene profile comprising one or more of increased S-linalool production, increased terpinolene production, increased β-ocimene production, α-pinene production greater than β-pinene production, reduced or lack of R-linalool production, reduced or lack of α-terpineol production and reduced or lack of fenchol production compared to at least one of the parent cultivars.

A34.10. The method of any one of embodiments A34 to A34.9 that is a method of breeding for one or more offspring cultivars that produces an increased sedative effect compared to at least one of the parent cultivars.

A34.11. The method of embodiment A34.10, wherein the one or more offspring cultivars comprises a terpene profile comprising one or more of: about equal or equal amounts of β-pinene and α-pinene production, increased R-linalool production, increased limonene production, increased trans-nerolidol production, increased terpineol production, increased camphene production, reduced or lack of β-ocimene production, reduced or lack of S-linalool production and reduced or lack of terpinolene production compared to at least one of the parent cultivars.

A34.12. The method of any one of embodiments A34 to A34.11 that is a method of breeding for one or more offspring cultivars that produces an increased cognitive-enhancing effect compared to at least one of the parent cultivars.

A34.13. The method of embodiment A34.12, wherein the one or more offspring cultivars comprises a terpene profile comprising one or more of: greater amounts of β-pinene production relative to α-pinene production, increased β-ocimene production and increased eucalyptol production compared to at least one of the parent cultivars.

A34.14. The method of any one of embodiments A34 to A34.13 that is a method of breeding for one or more offspring cultivars that produces an increased appetite-suppressing effect compared to at least one of the parent cultivars.

A34.15. The method of embodiment A34.14, wherein the one or more offspring cultivars comprises a terpene profile comprising increased amounts of humulene production compared to at least one of the parent cultivars.

A34.16. The method of any one of embodiments A34 to A34.15 that is a method of breeding for one or more offspring cultivars that produces an increased anti-inflammatory effect compared to at least one of the parent cultivars.

A34.17. The method of embodiment A34.16, wherein the one or more offspring cultivars comprises a terpene profile comprising one or more of: increased α-pinene production, increased humulene production and increased β-caryophyllene production compared to at least one of the parent cultivars.

A34.18. The method of any one of embodiments A34 to A34.17 that is a method of breeding for one or more offspring cultivars that produces an increased anti-anxiety effect compared to at least one of the parent cultivars.

A34.19. The method of embodiment A34.18, wherein the one or more offspring cultivars comprises a terpene profile comprising one or more of: increased β-pinene production, increased humulene production, increased β-caryophyllene production, increased linalool production, increased nerolidol production and increased limonene production compared to at least one of the parent cultivars.

A34.20. The method of any one of embodiments A34 to A34.19 that is a method of breeding for one or more offspring cultivars that produces an increased antinociceptive effect compared to at least one of the parent cultivars.

A34.21. The method of embodiment A34.20, wherein the one or more offspring cultivars comprises a terpene profile comprising one or more of: increased α-bisabolol production, increased α-terpineol production, increased trans nerolidol production, increased α-phellandrene production, and increased eucalyptol production compared to at least one of the parent cultivars.

A34.22. The method of any one of embodiments A34 to A34.21 that is a method of breeding for one or more offspring cultivars that produces an increased body relaxing effect compared to at least one of the parent cultivars.

A34.23. The method of embodiment A34.22, wherein the one or more offspring cultivars comprises a terpene profile comprising one or more of: increased α-bisabolol production, increased α-terpineol production, increased trans nerolidol production and increased α-phellandrene production, compared to at least one of the parent cultivars.

A34.24. The method of any one of embodiments A34 to A34.23 that is a method of breeding for one or more offspring cultivars that produces an increased anti-depressant effect compared to at least one of the parent cultivars.

A34.25. The method of embodiment A34.24, wherein the one or more offspring cultivars comprises a terpene profile comprising one or more of: equal or about equal amounts of α-pinene and β-pinene production, increased limonene production, increased nerolidol production and increased linalool production, compared to at least one of the parent cultivars.

A34.26. The method of any one of embodiments A34 to A34.25 that is a method of breeding for one or more offspring cultivars that produces an increased amount of one or more acetyl cholinesterase-inhibitor (AChEI) terpenes compared to at least one of the parent cultivars.

A34.27. The method of embodiment A34.26, wherein the one or more offspring cultivars comprises a terpene profile comprising one or more of: increased amounts of α-pinene production, increased terpinolene production, increased β-ocimene production, increased 3-carene production, increased α and/or γ-terpinene production and increased sabinene production compared to at least one of the parent cultivars.

A34.28. The method of any one of embodiments A34 to A34.27 that is a method of breeding for one or more offspring cultivars that produces an increased anti-bacterial effect compared to at least one of the parent cultivars.

A34.29. The method of embodiment A34.28, wherein the one or more offspring cultivars comprises a terpene profile comprising one or more of: increased amounts of aromadendrene production, increased carvacrol production, increased β-caryophyllene production, increased eucalyptol production, increased fenchol production, increased germacrene D production, increased nerol production, increased pulegone production, increased sabinene production and increased geraniol production compared to at least one of the parent cultivars.

A34.30. The method of any one of embodiments A34 to A34.29 that is a method of breeding for one or more offspring cultivars that produces an increased anti-microbial effect compared to at least one of the parent cultivars.

A34.31. The method of embodiment A34.30, wherein the one or more offspring cultivars comprises a terpene profile comprising one or more of: increased amounts of camphor production, increased sabinene hydrate production and increased thymol production compared to at least one of the parent cultivars.

A34.32. The method of any one of embodiments A34 to A34.31 that is a method of breeding for one or more offspring cultivars that produces an increased fungicidal effect compared to at least one of the parent cultivars.

A34.33. The method of embodiment A34.32, wherein the one or more offspring cultivars comprises a terpene profile comprising one or more of: increased amounts of citronellol production, increased para-cymene production, increased pulegone production and increased geraniol production compared to at least one of the parent cultivars.

A34.34. The method of any one of embodiments A34 to A34.33 that is a method of breeding for one or more offspring cultivars that produces an increased expectorant effect compared to at least one of the parent cultivars.

A34.35. The method of embodiment A34.34, wherein the one or more offspring cultivars comprises a terpene profile comprising one or more of: increased amounts of camphene production, increased sabinene hydrate production and increased geraniol production compared to at least one of the parent cultivars.

A34.36. The method of any one of embodiments A34 to A34.35 that is a method of breeding for one or more offspring cultivars that produces an increased expectorant effect compared to at least one of the parent cultivars.

A34.37. The method of embodiment A34.36, wherein the one or more offspring cultivars comprises a terpene profile comprising one or more of: increased amounts of camphene production, increased sabinene hydrate production and increased geraniol production compared to at least one of the parent cultivars.

A34.38. The method of any one of embodiments A34 to A34.37 that is a method of breeding for one or more offspring cultivars that produces increased non-irritant properties compared to at least one of the parent cultivars.

A34.39. The method of embodiment A34.38, wherein the one or more offspring cultivars comprises a terpene profile comprising one or more of reduced or absent: borneol, α-cedrene, citronellol or para-cymene, or increased amounts of the counter-irritant fenchone.

A35. The method of any one of embodiments A32 to A34, wherein the plant cultivar is selected for resistance to an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof.

A35.1. The method of embodiment A35, wherein the plant cultivar is selected for resistance to an organism or situation that is identified based on identifying one or more terpene synthase genes, determining the expression profile of one or more terpene synthase genes, and/or determining the production profile of one or more terpenes.

A35.2. The method of any one of embodiments A35 or A35.1, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting resistance to an organism or situation to a plant cultivar.

A35.3. The method of any one of embodiments A35 to A35.2, wherein the one or more TPS genes, or sets thereof, produce one or more of the terpenes selected from among aα-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

A35.4. The method of any one of embodiments A35 to A35.3 that is a method of breeding for one or more offspring cultivars that show increased anti-pathogenic properties compared to at least one of the parent cultivars.

A35.5. The method of embodiment A35.4, wherein the one or more offspring cultivars comprises a terpene synthase profile comprising one or more of increased amounts/expression of TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37JL.

A35.6. The method of any one of embodiments A35 to A35.5 that is a method of breeding for one or more offspring cultivars comprising one or more root specifically expressed terpene synthases that increase resistance against pests in the soil and/or one or more root specifically expressed terpene synthases that respond favorably to beneficial microorganisms in the soil, compared to at least one of the parent cultivars.

A35.7. The method of embodiment A35.6, wherein the one or more offspring cultivars comprises a terpene synthase profile comprising one or more of increased amounts/expression of TPS11JL, TPS49JL, TPS41JL, TPS12JL, TPS11-likeJL, TPS36JL, TPS6JL, TPS37JL and TPS64JL.

A35.8. The method of any one of embodiments A35 to A35.7 that is a method of breeding for one or more offspring cultivars comprising one or more stem specifically expressed terpene synthases that increase resistance against stem-hosted pests, compared to at least one of the parent cultivars.

A35.9. The method of embodiment A35.8, wherein the one or more offspring cultivars comprises a terpene synthase profile comprising one or more of increased amounts/expression of TPS63JL, TPS43JL, TPS41JL, TPS6-likeJL, TPS33JL and TPS24JL.

A35.10. The method of any one of embodiments A35 to A35.9 that is a method of breeding for one or more offspring cultivars comprising one or more herbicidal properties, compared to at least one of the parent cultivars.

A35.11. The method of embodiment A35.10, wherein the one or more offspring cultivars comprises a terpene profile comprising one or more of increased amounts of geraniol, pilegone, citronellol, borneol and para-cymene.

A35.12. The method of any one of embodiments A35 to A35.11 that is a method of breeding for one or more offspring cultivars comprising that comprise one or more pesticidal properties, compared to at least one of the parent cultivars.

A35.13. The method of embodiment A35.12, wherein the one or more offspring cultivars comprises a terpene profile comprising one or more of increased amounts of aromadendrene, α-bisabolol, cedrol, nerolidol, trans-nerolidol and guaiol.

A36. The method of any one of embodiments A32 to A34, wherein the plant cultivar is selected for having an affinity towards an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof.

A36.1. The method of embodiment A36 that is a method of breeding for one or more offspring cultivars comprising one or more insect pheromonal properties, compared to at least one of the parent cultivars.

A36.2. The method of embodiment A36.1, wherein the one or more offspring cultivars comprises a terpene profile comprising one or more of increased amounts of endo-borneol, isoborneol, 3-carene, carveol, germacrene B, hedycaryol, menthol, cis-nerolidol, cis-β-ocimene, trans-β-ocimene, sabinene hydrate, α-terpinene, thymol, β-farnesene, α-farnesene, γ-eudesmol, alloaromadendrene, valencene and pulegone.

A37. The method of embodiment A35 or A36.2, wherein the organism or situation is selected from among exposure to insects, pests, mold, chemicals, mildew, fungi, bacteria, an environmental condition or a geographic location.

A38. The method of any one of embodiments A32 to A37, wherein the plant cultivar is selected for root-specific, stem-specific, leaf-specific or flower-specific expression of a terpene synthase gene and/or paralog thereof, a terpene, a cannabinoid or a flavonoid based on identifying one or more terpene synthase genes, determining the expression profile of one or more terpene synthase genes, determining the production profile of one or more terpenes, determining the production profile of one or more cannabinoids, determining the production profile of one or more flavonoids or a combination thereof.

A39. The method of any one of embodiments A1 to A31, further comprising, based on identifying one or more terpene synthase genes and/or paralogs thereof, determining the expression profile of the one or more terpene synthase genes and/or paralogs thereof, determining the production profile of one or more terpenes, determining the production profile of one or more cannabinoids, determining the production profile of one or more flavonoids or a combination thereof, genetically modifying a plant cultivar whereby the expression of at least one terpene synthase gene and/or paralog thereof is inhibited or increased in the plant cultivar.

A40. The method of embodiment A39, wherein the genetic modification increases the production of at least one terpene or decreases the production of at least one terpene in the plant cultivar.

A41. The method of embodiment A39 or A40, wherein the plant cultivar is of a Cannabis cultivar.

A41.1. The method of embodiment A39 or A40, wherein one or more plant cultivars is/are of the family Rosidae.

A41.2. The method of embodiment A41, whereib the Cannabis cultivar is selected from among one or more of Type 1, Type 2, Type 3, Type 4 and Type 5 cultivars

A42. The method of any one of embodiments A41 to A41.2, wherein the genetic modification increases the production of at least one cannabinoid or decreases the production of at least one cannabinoid in the plant cultivar.

A43. The method of any one of embodiments A39 to A42, wherein the genetic modification is for imparting a medicinal use.

A44. The method of any one of embodiments A39 to A43, wherein the genetic modification is for imparting resistance to an organism or situation.

A45. The method of any one of embodiments A39 to A43, wherein the genetic modification is for imparting affinity towards an organism or situation.

A46. The method of embodiment A44 or A45, wherein the organism or situation is selected from among exposure to insects, pests, chemicals, mold, mildew, fungi, bacteria, an environmental condition or a geographic location.

A47. The method of any one of embodiments A39 to A46, wherein the genetic modification is for imparting root-specific, stem-specific, leaf-specific or flower-specific expression or inhibition of expression of a terpene synthase gene, a terpene, a cannabinoid or a flavonoid.

A48. The method of any one of embodiments A39 to A47, wherein the genetic modification is by a method comprising CRISPR-cas, Cre-Lox, MiRNA, SiRNA, ShRNA or a combination thereof.

A49. The method of any one of embodiments A1 to A48, wherein the unique subsequence of at least one terpene synthase gene or paralog thereof is outside the sequence encoding the active site of the terpene synthase gene or paralog thereof.

A50. The method of any one of embodiments A1 to A48, wherein the unique subsequence of at least one terpene synthase gene or paralog thereof is within the sequence encoding the active site of the terpene synthase gene or paralog thereof.

B1. A method of producing a daughter plant cultivar, comprising:
analyzing two or more parent plant cultivars by the method of any one of embodiments A1 to A50;
based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting two parent plant cultivars for producing a desired daughter plant cultivar; and
inbreeding or outcrossing the parent plant cultivars to produce a daughter plant cultivar.

B2. The method of embodiment B1, wherein the daughter plant cultivar produced has increased expression of at least one terpene synthase gene and/or paralog thereof or decreased expression of at least one terpene synthase gene and/or paralog thereof compared to at least one of the parent plant cultivars.

B3. The method of embodiment B1 or B2, wherein the daughter plant cultivar produced has increased production of at least one terpene or decreased production of at least one terpene compared to at least one of the parent plant cultivars.

B4. The method of any one of embodiments B1 to B3, wherein the parent plant cultivars and the daughter plant cultivar are Cannabis cultivars.

B4.1. The method of any one of embodiments B1 to B3, wherein the parent plant cultivars and the daughter plant cultivar is/are of the family Rosidae.

B4.2. The method of embodiment B4, whereib the Cannabis cultivar is selected from among one or more of Type 1, Type 2, Type 3, Type 4 and Type 5 cultivars.

B5. The method of any one of embodiments B4 to B4.2, wherein the daughter plant cultivar produced has increased production of at least one cannabinoid or decreased production of at least one cannabinoid compared to at least one of the parent plant cultivars.

B6. The method of any one of embodiments B1 to B5, wherein the daughter plant cultivar has a medicinal use that is reduced or absent in the parent plant cultivars.

B6.1. The method of embodiment AB6, wherein the plant cultivar is selected for a medicinal use that is assigned based on identifying one or more terpene synthase genes, determining the expression profile of one or more terpene synthase genes, and/or determining the production profile of one or more terpenes.

B6.2. The method of embodiment B6 or B6.1, whwrein the medicinal use is selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

B6.3. The method of embodiment B6 or B6.1, wherein the medicinal use is to impart energy, mental clarity, appetite stimulation or appetite suppression.

B6.4. The method of any one of embodiments B6 to B6.3, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting one or more medicinal uses to a plant cultivar.

B6.5. The method of any one of embodiments B6 to B6.4, wherein the one or more TPS genes, or sets thereof, produce one or more of the terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, yγ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

B6.6. The method of any one of embodiments B6 to B6.5 that is a method of breeding for one or more offspring cultivars that show increased cannabinoid production compared to at least one of the parent cultivars.

B6.7. The method of embodiment B6.6, wherein the one or more offspring cultivars show reduced expression, or lack of expression, of one or more terpene synthases selected from among TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL and TPS37JL compared to at least one of the parent cultivars.

B6.8. The method of any one of embodiments B6 to B6.7 that is a method of breeding for one or more offspring cultivars that produces an increased energetic effect compared to at least one of the parent cultivars.

B6.9. The method of embodiment B6.8, wherein the one or more offspring cultivars comprises a terpene profile comprising one or more of increased S-linalool production, increased terpinolene production, increased β-ocimene production, α-pinene production greater than β-pinene production, reduced or lack of R-linalool production, reduced or lack of α-terpineol production and reduced or lack of fenchol production compared to at least one of the parent cultivars.

B6.10. The method of any one of embodiments B6 to B6.9 that is a method of breeding for one or more offspring cultivars that produces an increased sedative effect compared to at least one of the parent cultivars.

B6.11. The method of embodiment B6.10, wherein the one or more offspring cultivars comprises a terpene profile comprising one or more of: about equal or equal amounts of β-pinene and α-pinene production, increased R-linalool production, increased limonene production, increased trans-nerolidol production, increased terpineol production, increased camphene production, reduced or lack of β-ocimene production, reduced or lack of S-linalool production and reduced or lack of terpinolene production compared to at least one of the parent cultivars.

B6.12. The method of any one of embodiments B6 to B6.11 that is a method of breeding for one or more offspring cultivars that produces an increased cognitive-enhancing effect compared to at least one of the parent cultivars.

B6.13. The method of embodiment B6.12, wherein the one or more offspring cultivars comprises a terpene profile comprising one or more of: greater amounts of β-pinene production relative to α-pinene production, increased β-ocimene production and increased eucalyptol production compared to at least one of the parent cultivars.

B6.14. The method of any one of embodiments B6 to B6.13 that is a method of breeding for one or more offspring cultivars that produces an increased appetite-suppressing effect compared to at least one of the parent cultivars.

B6.15. The method of embodiment B6.14, wherein the one or more offspring cultivars comprises a terpene profile comprising increased amounts of humulene production compared to at least one of the parent cultivars.

B6.16. The method of any one of embodiments B6 to B6.15 that is a method of breeding for one or more offspring cultivars that produces an increased anti-inflammatory effect compared to at least one of the parent cultivars.

B6.17. The method of embodiment B6.16, wherein the one or more offspring cultivars comprises a terpene profile comprising one or more of: increased α-pinene production, increased humulene production and increased β-caryophyllene production compared to at least one of the parent cultivars.

B6.18. The method of any one of embodiments B6 to B6.17 that is a method of breeding for one or more offspring cultivars that produces an increased anti-anxiety effect compared to at least one of the parent cultivars.

B6.19. The method of embodiment B6.18, wherein the one or more offspring cultivars comprises a terpene profile comprising one or more of: increased β-pinene production, increased humulene production, increased β-caryophyllene production, increased linalool production, increased nerolidol production and increased limonene production compared to at least one of the parent cultivars.

B6.20. The method of any one of embodiments B6 to B6.19 that is a method of breeding for one or more offspring cultivars that produces an increased antinociceptive effect compared to at least one of the parent cultivars.

B6.21. The method of embodiment B6.20, wherein the one or more offspring cultivars comprises a terpene profile comprising one or more of: increased α-bisabolol production, increased α-terpineol production, increased trans nerolidol production, increased α-phellandrene production, and increased eucalyptol production compared to at least one of the parent cultivars.

B6.22. The method of any one of embodiments B6 to B6.21 that is a method of breeding for one or more offspring cultivars that produces an increased body relaxing effect compared to at least one of the parent cultivars.

B6.23. The method of embodiment B6.22, wherein the one or more offspring cultivars comprises a terpene profile comprising one or more of: increased α-bisabolol production, increased α-terpineol production, increased trans nerolidol production and increased α-phellandrene production, compared to at least one of the parent cultivars.

B6.24. The method of any one of embodiments B6 to B6.23 that is a method of breeding for one or more offspring cultivars that produces an increased anti-depressant effect compared to at least one of the parent cultivars.

B6.25. The method of embodiment B6.24, wherein the one or more offspring cultivars comprises a terpene profile comprising one or more of: equal or about equal amounts of α-pinene and β-pinene production, increased limonene production, increased nerolidol production and increased linalool production, compared to at least one of the parent cultivars.

B6.26. The method of any one of embodiments B6 to B6.25 that is a method of breeding for one or more offspring cultivars that produces an increased amount of one or more acetyl cholinesterase-inhibitor (AChEI) terpenes compared to at least one of the parent cultivars.

B6.27. The method of embodiment B6.26, wherein the one or more offspring cultivars comprises a terpene profile comprising one or more of: increased amounts of α-pinene production, increased terpinolene production, increased β-ocimene production, increased 3-carene production, increased α and/or γ-terpinene production and increased sabinene production compared to at least one of the parent cultivars.

B6.28. The method of any one of embodiments B6 to B6.27 that is a method of breeding for one or more offspring cultivars that produces an increased anti-bacterial effect compared to at least one of the parent cultivars.

B6.29. The method of embodiment B6.28, wherein the one or more offspring cultivars comprises a terpene profile comprising one or more of: increased amounts of aromadendrene production, increased carvacrol production, increased β-caryophyllene production, increased eucalyptol production, increased fenchol production, increased germacrene D production, increased nerol production, increased pulegone production, increased sabinene production and increased geraniol production compared to at least one of the parent cultivars.

B6.30. The method of any one of embodiments B6 to B6.29 that is a method of breeding for one or more offspring cultivars that produces an increased anti-microbial effect compared to at least one of the parent cultivars.

B6.31. The method of embodiment B6.30, wherein the one or more offspring cultivars comprises a terpene profile comprising one or more of: increased amounts of camphor production, increased sabinene hydrate production and increased thymol production compared to at least one of the parent cultivars.

B6.32. The method of any one of embodiments B6 to B6.31 that is a method of breeding for one or more offspring cultivars that produces an increased fungicidal effect compared to at least one of the parent cultivars.

B6.33. The method of embodiment B6.32, wherein the one or more offspring cultivars comprises a terpene profile comprising one or more of: increased amounts of citronellol production, increased para-cymene production, increased pulegone production and increased geraniol production compared to at least one of the parent cultivars.

B6.34. The method of any one of embodiments B6 to B6.33 that is a method of breeding for one or more offspring cultivars that produces an increased expectorant effect compared to at least one of the parent cultivars.

B6.35. The method of embodiment B6.34, wherein the one or more offspring cultivars comprises a terpene profile comprising one or more of: increased amounts of camphene production, increased sabinene hydrate production and increased geraniol production compared to at least one of the parent cultivars.

B6.36. The method of any one of embodiments B6 to B6.35 that is a method of breeding for one or more offspring cultivars that produces an increased expectorant effect compared to at least one of the parent cultivars.

B6.37. The method of embodiment B6.36, wherein the one or more offspring cultivars comprises a terpene profile comprising one or more of: increased amounts of camphene production, increased sabinene hydrate production and increased geraniol production compared to at least one of the parent cultivars.

B6.38. The method of any one of embodiments B6 to B6.37 that is a method of breeding for one or more offspring cultivars that produces increased non-irritant properties compared to at least one of the parent cultivars.

B6.39. The method of embodiment B6.38, wherein the one or more offspring cultivars comprises a terpene profile comprising one or more of reduced or absent: borneol, α-cedrene, citronellol or para-cymene, or increased amounts of the counter-irritant fenchone.

B7. The method of any one of embodiments B1 to B6, wherein the daughter plant cultivar has resistance to an organism or situation, where the resistance is reduced or absent in the parent plant cultivars.

B7.1. The method of embodiment B7, wherein the plant cultivar is selected for resistance to an organism or situation that is identified based on identifying one or more terpene synthase genes, determining the expression profile of one or more terpene synthase genes, and/or determining the production profile of one or more terpenes.

B7.2. The method of any one of embodiments B7 or B7.1, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting resistance to an organism or situation to a plant cultivar.

B7.3. The method of any one of embodiments B7 to B7.2, wherein the one or more TPS genes, or sets thereof, produce one or more of the terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

B7.4. The method of any one of embodiments B7 to B7.3 that is a method of breeding for one or more offspring cultivars that show increased anti-pathogenic properties compared to at least one of the parent cultivars.

B7.5. The method of embodiment B7.4, wherein the one or more offspring cultivars comprises a terpene synthase profile comprising one or more of increased amounts/expression of TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37JL.

B7.6. The method of any one of embodiments B7 to B7.5 that is a method of breeding for one or more offspring cultivars comprising one or more root specifically expressed terpene synthases that increase resistance against pests in the soil and/or one or more root specifically expressed terpene synthases that respond favorably to beneficial microorganisms in the soil, compared to at least one of the parent cultivars.

B7.7. The method of embodiment B7.6, wherein the one or more offspring cultivars comprises a terpene synthase profile comprising one or more of increased amounts/expression of TPS11JL, TPS49JL, TPS41JL, TPS12JL, TPS11-likeJL, TPS36JL, TPS6JL, TPS37JL and TPS64JL.

B7.8. The method of any one of embodiments B7 to B7.7 that is a method of breeding for one or more offspring cultivars comprising one or more stem specifically expressed terpene synthases that increase resistance against stem-hosted pests, compared to at least one of the parent cultivars.

B7.9. The method of embodiment B7.8, wherein the one or more offspring cultivars comprises a terpene synthase profile comprising one or more of increased amounts/expression of TPS63JL, TPS43JL, TPS41JL, TPS6-likeJL, TPS33JL and TPS24JL.

B7.10. The method of any one of embodiments B7 to B7.9 that is a method of breeding for one or more offspring cultivars comprising one or more herbicidal properties, compared to at least one of the parent cultivars.

B7.11. The method of embodiment B7.10, wherein the one or more offspring cultivars comprises a terpene profile comprising one or more of increased amounts of geraniol, pilegone, citronellol, borneol and para-cymene.

B7.12. The method of any one of embodiments B7 to B7.11 that is a method of breeding for one or more offspring cultivars comprising that comprise one or more pesticidal properties, compared to at least one of the parent cultivars.

B7.13. The method of embodiment B7.12, wherein the one or more offspring cultivars comprises a terpene profile comprising one or more of increased amounts of aromadendrene, α-bisabolol, cedrol, nerolidol, trans-nerolidol and guaiol.

B8. The method of any one of embodiments B1 to B6, wherein the daughter plant cultivar has affinity towards an organism or situation, where the affinity is reduced or absent in the parent plant cultivars.

B8.1. The method of embodiment B8 that is a method of breeding for one or more offspring cultivars comprising one or more insect pheromonal properties, compared to at least one of the parent cultivars.

B8.2. The method of embodiment B8.1, wherein the one or more offspring cultivars comprises a terpene profile comprising one or more of increased amounts of endo-borneol, isoborneol, 3-carene, carveol, germacrene B, hedycaryol, menthol, cis-nerolidol, cis-β-ocimene, trans-β-ocimene, sabinene hydrate, α-terpinene, thymol, β-farnesene, α-farnesene, γ-eudesmol, alloaromadendrene, valencene and pulegone.

B9. The method of embodiment B7 or B8, wherein the organism or situation is selected from among exposure to insects, pests, mold, chemicals, mildew, fungi, bacteria, an environmental condition or a geographic location.

B10. The method of any one of embodiments B1 to B9, wherein the daughter plant cultivar has increased root-specific, stem-specific, leaf-specific or flower-specific expression or inhibition of expression of a terpene synthase gene, a terpene, a cannabinoid or a flavonoid compared to at least one of the parent plant cultivars.

C1. A method of genetically modifying a plant cultivar, comprising:
analyzing the plant cultivar by the method of any one of embodiments A1 to A49.1; and
based on the analysis, altering at least one unique subsequence of at least one terpene synthase gene or a paralog thereof, whereby expression of the at least one terpene synthase gene or paralog thereof is increased or decreased compared to in the absence of the alteration and whereby one or more of the terpene synthase gene and/or paralog thereof expression profile, the terpene production profile, the cannabinoid production profile or the flavonoid production profile of the plant is modified.

C2. The method of embodiment C1, wherein the genetically modified plant cultivar has increased expression of at least one terpene synthase gene or a paralog thereof or decreased expression of at least one terpene synthase gene or a paralog thereof, compared to the unmodified plant cultivar.

C3. The method of embodiment C1 or C2, wherein the genetically modified plant cultivar has increased production of at least one terpene or decreased production of at least one terpene compared to the unmodified plant cultivar.

C4. The method of any one of embodiments C1 to C3, wherein the plant cultivar that is genetically modified is of a Cannabis cultivar.

C5. The method of embodiment C4, wherein the genetically modified plant cultivar has increased production of at least one cannabinoid or decreased production of at least one cannabinoid compared to the unmodified plant cultivar.

C6. The method of any one of embodiments C1 to C5, wherein the genetically modified plant cultivar has a medicinal use that is less than or absent in the unmodified plant cultivar.

C7. The method of any one of embodiments C1 to C6, wherein the genetically modified plant cultivar has increased resistance to an organism or situation, where the resistance is less than or absent in the unmodified plant cultivar.

C8. The method of any one of embodiments C1 to C6, wherein the genetically modified plant cultivar has increased affinity towards an organism or situation, where the affinity is less than or absent in the unmodified plant cultivar.

C9. The method of embodiment C7 or C8, wherein the organism or situation is selected from among exposure to insects, pests, mold, chemicals, mildew, fungi, bacteria, an environmental condition or a geographic location.

010. The method of any one of embodiments C1 to C9, wherein the genetically modified plant cultivar has increased root-specific, stem-specific, leaf-specific or flower-specific expression or inhibition of expression of a terpene synthase gene, a terpene, a cannabinoid or a flavonoid compared to the unmodified plant cultivar.

C11. The method of any one of embodiments C1 to 010, wherein the genetic modification is by a method comprising CRISPR-cas, Cre-Lox, MiRNA, SiRNA, ShRNA or a combination thereof.

C12. The method of any one of embodiments C1 to C11, wherein the expression of two or more terpene synthase genes and/or paralogs thereof is specifically increased or specifically inhibited.

D1. A method of analyzing a gene of a plant cultivar that belongs to a family of genes, wherein the gene comprises two or more exons, the method comprising:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique exon of the gene or a portion thereof, wherein the unique exon of the gene is different than the other exons of the gene and the unique exon of the gene is different than the exons of the other genes in the gene family;
(c) amplifying the mixture, thereby obtaining an amplified mixture; and
(d) analyzing the amplified mixture of (c), whereby the gene of the plant cultivar is identified and/or quantified in the amplified mixture.

D1.1 A method of analyzing a family of genes of a plant cultivar, wherein each gene of the family comprises two or more exons, the method comprising:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique exon of a gene or a portion thereof, wherein the unique exon of the gene is different than the other exons of the gene and the unique exon of the gene is different than the exons of the other genes in the family of genes;
(c) amplifying the mixture, thereby obtaining an amplified mixture; and
(d) analyzing the amplified mixture of (c), whereby at least one gene of the family of genes is identified and/or quantified in the amplified mixture.

D2. The method of embodiment D1 or D1.1, wherein the family of genes comprises terpene synthase genes and/or paralogs thereof.

E1. A solid support comprising a single-stranded polynucleotide species, wherein the single-stranded polynucleotide species specifically binds to a unique subsequence of a terpene synthase gene or a paralog thereof, wherein the unique subsequence of the terpene synthase gene or paralog thereof is different than the other subsequences of the terpene synthase gene or paralog thereof and the unique subsequence of the terpene synthase gene or paralog thereof is different than the subsequences of other terpene synthase genes and/or paralogs thereof.

E2. The solid support of embodiment E1, wherein the single-stranded polynucleotide species specifically binds to a conserved region of the unique subsequence.

E3. The solid support of embodiment E1 or E2, wherein the unique subsequence is an exon, an intron, a portion within an exon or a portion within an intron.

E4. The solid support of any one of embodiments E1 to E3, wherein the unique subsequence is an exon or a portion within an exon.

E5. The solid support of any one of embodiments E1 to E4, wherein the single-stranded polynucleotide species is selected from among SEQ ID NOS: 1-1284.

E5.1 The solid support of any one of embodiments E1 to E5, wherein the single-stranded polynucleotide species is selected from among those set forth in SEQ ID NOS: 1-1284, or from among sequences that share 90%, 91%, 92%, 93%, 94%, 95%, 96%, 97%, 98%, 99% or more identity with any of the sequences set forth in SEQ ID NOS: 1-1284.

E6. The solid support of any one of embodiments E1 to E5.1, wherein the terpene synthase gene or paralog thereof is a monoterpene synthase gene or paralog thereof, a diterpene synthase gene or paralog thereof or a sesquiterpene synthase gene or paralog thereof.

E7. The solid support of any one of embodiments E1 to E6, wherein the single-stranded polynucleotide species specifically binds to a unique subsequence of a terpene synthase gene or a paralog thereof from a Cannabis cultivar.

E8. The solid support of any one of embodiments E1 to E7 that is a bead, column, capillary, disk, filter, dipstick, membrane, wafer, comb, pin or a chip.

E9. The solid support of any one of embodiments E1 to E8 that comprises a material selected from among silicon, silica, glass, controlled-pore glass (CPG), nylon, Wang resin, Merrifield resin, Sephadex, Sepharose, cellulose, magnetic beads, Dynabeads, a metal, a metal surface, a plastic or polymer or combinations thereof.

E10. The solid support of any one of embodiments E1 to E9, wherein the unique subsequence of at least one terpene synthase gene or paralog thereof is outside the sequence encoding the active site of the terpene synthase gene or paralog thereof.

E11. The solid support of any one of embodiments E1 to E9, wherein the unique subsequence of at least one terpene synthase gene or paralog thereof is within the sequence encoding the active site of the terpene synthase gene or paralog thereof.

F1. A collection of solid supports of any one of embodiments E1 to E10, wherein:
(a) each solid support in the collection comprises a single-stranded polynucleotide species, wherein the single-stranded polynucleotide species specifically binds to a unique subsequence of a terpene synthase gene or a paralog thereof, wherein the unique subsequence of the terpene synthase gene or paralog thereof is different than the other subsequences of the terpene synthase gene or paralog thereof and the unique subsequence of the terpene synthase gene or paralog thereof is different than the subsequences of other terpene synthase genes and/or paralogs thereof; and
(b) the single-stranded polynucleotide species of each solid support in the collection is different than the single-stranded polynucleotide species of the other solid supports in the collection.

F2. The collection of embodiment F1, wherein each single-stranded polynucleotide species in the collection specifically binds to a unique subsequence of the same terpene synthase gene or paralog thereof.

F3. The collection of embodiment F1, wherein each single-stranded polynucleotide species specifically binds to a unique subsequence of a terpene synthase gene or a paralog thereof that is different than the terpene synthase genes and/or paralogs thereof to which the other single-stranded polynucleotide species in the collection bind.

F4. The collection of embodiment F1, comprising at least two single-stranded polynucleotide species that specifically bind to unique subsequences of the same terpene synthase gene and/or paralog thereof or at least two single-stranded polynucleotide species that specifically bind to unique subsequences of two different terpene synthase genes and/or paralogs thereof.

F5. The collection of any one of embodiments F1 to F4, wherein each of the single-stranded polynucleotide species specifically binds to a conserved region of the unique subsequence.

F6. The collection of any one of embodiments F1 to F5, wherein the unique subsequence is an exon, an intron, a portion within an exon or a portion within an intron.

F7. The collection of any embodiments F1 to F6, wherein the subsequence is an exon or a portion within an exon.

F8. The collection of any one of embodiments F1 to F7, wherein the single-stranded polynucleotide species are selected from among SEQ ID NOS: 1-1284.

F8.1 The collection of any one of embodiments F1 to F8, wherein the single-stranded polynucleotide species are selected from among those set forth in SEQ ID NOS: 1-1284, or from among sequences that share 90%, 91%, 92%, 93%, 94%, 95%, 96%, 97%, 98%, 99% or more identity with any of the sequences set forth in SEQ ID NOS: 1-1284.

F9. The collection of any one of embodiments F1 to F8.1, wherein the terpene synthase genes and/or paralogs thereof are monoterpene synthase genes and/or paralogs thereof, diterpene synthase genes and/or paralogs thereof, sesquiterpene synthase genes and/or paralogs thereof, or any combination thereof.

F10. The collection of any one of embodiments F1 to F9, wherein all or a portion of the single-stranded polynucleotide species specifically binds to a unique subsequence of a terpene synthase gene or a paralog thereof from a Cannabis cultivar.

F11. The collection of any one of embodiments F1 to F10, wherein the solid supports are arranged in an array.

F12. The collection of embodiment F11, wherein the array is on a chip.

G1. A method of analyzing the terpene synthase gene profile of a plant cultivar, comprising:
(a) obtaining a nucleic acid sample from the plant cultivar;
(b) contacting the nucleic acid sample with the collection of any of embodiments F1 to F12, thereby preparing a mixture, wherein each single-stranded polynucleotide species of the collection can specifically bind to a unique subsequence of a terpene synthase gene or a paralog thereof, wherein the unique subsequence of the terpene synthase gene or a paralog thereof is different than the other subsequences of the terpene synthase gene or paralog thereof and the unique subsequence of the terpene synthase gene is different than the subsequences of other terpene synthase genes and/or paralogs thereof; and
(c) subjecting the mixture to conditions that facilitate specific binding of each single-stranded polynucleotide species of the collection to its corresponding unique subsequence of a terpene synthase gene or paralog thereof, when the unique subsequence is present in the nucleic acid sample, thereby obtaining a collection comprising bound single-stranded polynucleotide species; and
(d) analyzing the collection comprising bound single-stranded polynucleotide species of (c), whereby at least one terpene synthase gene or a paralog thereof is identified and/or quantified, and the terpene synthase gene profile of the plant cultivar is determined.

G2. The method of embodiment G1, wherein each single-stranded polynucleotide species of the collection binds to a conserved region of its corresponding unique subsequence.

G3. The method of embodiment G1 or G2, wherein the unique subsequence is an exon, an intron, a portion within an exon or a portion within an intron.

G4. The method of any one of embodiments G1 to G3, wherein the subsequence is an exon or a portion within an exon.

G5. The method of any one of embodiments G1 to G4, wherein the identification in (d) is by a signal that is generated when a single-stranded polynucleotide species of the collection binds to its corresponding unique subsequence.

G5.1 The method of embodiment G5, wherein the signal is an electrical signal, an electronic signal, from an optical label or from a radiolabel.

G6. The method of any one of embodiments G1 to G5.1, wherein the single-stranded polynucleotide species are selected from among SEQ ID NOS: 1-1284.

G6.1 The method of any one of embodiments G1 to G6, wherein the single-stranded polynucleotide species are selected from among those set forth in SEQ ID NOS: 1-1284, or from among sequences that share 90%, 91%, 92%, 93%, 94%, 95%, 96%, 97%, 98%, 99% or more identity with any of the sequences set forth in SEQ ID NOS: 1-1284.

G7. The method of any one of embodiments G1 to G6.1, wherein the terpene synthase genes and/or paralogs thereof that are identified and/or quantified in the terpene synthase gene profile are monoterpene synthase genes and/or paralogs thereof, diterpene synthase genes and/or paralogs thereof, sesquiterpene synthase genes and/or paralogs thereof or any combination thereof.

G8. The method of any one of embodiments G1 to G7, wherein, based on the terpene synthase gene profile that is obtained, the terpene synthase gene and/or paralog expression profile and/or the terpene production profile of the plant cultivar is determined.

G9. The method of embodiment G8, wherein the terpene synthase gene expression profile and/or the terpene production profile is of the root, flower, stem, leaf or any combination thereof.

G10. The method of any one of embodiments G1 to G9, wherein, based on the terpene synthase gene profile that is obtained and/or based on the terpene synthase gene expression profile that is determined and/or based on the terpene production profile that is determined, a lineage of the plant cultivar is assigned.

G11. The method of any one of embodiments G1 to G10, wherein, based on the terpene synthase gene profile that is obtained and/or based on the terpene synthase gene expression profile that is determined and/or based on the terpene production profile that is determined, a medicinal use of the plant cultivar is assigned.

G12. The method of any one of embodiments G1 to G11, wherein, based on the terpene synthase gene profile that is obtained and/or based on the terpene synthase gene expression profile that is determined and/or based on the terpene production profile that is determined, the plant cultivar is identified as resistant to an organism or situation.

G13. The method of embodiment G12, wherein the organism or situation is selected from among exposure to insects, pests, chemicals, mold, mildew, fungi, bacteria, an environmental condition or a geographic location.

G14. The method of any one of embodiments G1 to G11, wherein, based on the terpene synthase gene profile that is obtained and/or based on the terpene synthase gene and/or paralog expression profile that is determined and/or based on the terpene production profile that is determined, the plant cultivar is identified as having affinity towards an organism or situation.

G15. The method of embodiment G14, wherein the organism or situation is selected from among exposure to insects, pests, chemicals, mold, mildew, fungi, bacteria, an environmental condition or a geographic location.

G16. The method of any one of embodiments G1 to G15, wherein a plurality of plant cultivars are analyzed.

G17. The method of embodiment G16, wherein the plant cultivars are of the same species.

G18. The method of embodiment G16 or G17, comprising classifying the plurality of plant cultivars based on lineage.

G19. The method of any one of embodiments G16 to G18, comprising classifying the plurality of plant cultivars based on medicinal use.

G20. The method of any one of embodiments G1 to G19, wherein one or more of the plant cultivars is/are a Cannabis cultivar.

G21. The method of embodiment G20, wherein the monoterpene synthase gene and/or paralog profile of the Cannabis plant cultivar is obtained and, based on the monoterpene synthase gene and/or paralog profile obtained and/or the expression profile of the identified and/or quantified monoterpene synthase genes and/or paralogs thereof, the terpene production profile, the cannabinoid production profile, the flavonoid production profile, or the combination of two or more of the terpene production profile, the cannabinoid production profile and the flavonoid production profile of the Cannabis plant cultivar is determined.

G22. The method of embodiment G21, wherein, based on the monoterpene synthase gene and/or paralog profile obtained, the expression profile of the identified and/or quantified monoterpene synthase genes and/or paralogs thereof, the cannabinoid production profile, the flavonoid production profile, or the cannabinoid production profile and the flavonoid production profile that is determined, a lineage of the Cannabis plant cultivar is assigned.

G23. The method of embodiment G21 or G22, wherein, based on the monoterpene synthase gene and/or paralog profile obtained, the expression profile of the identified and/or quantified monoterpene synthase genes and/or paralogs thereof, the cannabinoid production profile, the flavonoid production profile, or the cannabinoid production profile and the flavonoid production profile that is determined, a medicinal use of the Cannabis plant cultivar is assigned.

G24. The method of any one of embodiments G20 to G23, wherein a plurality of Cannabis plant cultivars are analyzed.

G25. The method of embodiment G24, comprising classifying the plurality of Cannabis plant cultivars based on lineage.

G26. The method of embodiment G24, comprising classifying the plurality of plant cultivars based on medicinal use.

G27. The method of any one of embodiments G1 to G26, wherein at least one plant cultivar that is analyzed produces one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

G28. The method of any one of embodiments G8 to G26, wherein a terpene production profile is determined for one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

G29. The method of any one of embodiments G1 to G28, wherein at least one plant cultivar that is analyzed expresses one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2.

G30. The method of any one of embodiments G1 to G29, further comprising, based on obtaining the terpene synthase gene and/or paralog profile, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for in-breeding or out-crossing.

G31. The method of embodiment G30, wherein the plant cultivar is selected for its lineage that is assigned based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof.

G32. The method of embodiment G30 or G31, wherein the plant cultivar is selected for a medicinal use that is assigned based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof.

G33. The method of any one of embodiments G30 to G32, wherein the plant cultivar is selected for resistance to an organism or situation that is identified based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof.

G34. The method of any one of embodiments G30 to G32, wherein the plant cultivar is selected for having an affinity towards an organism or situation that is identified based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof.

G35. The method of embodiment G33 or G34, wherein the organism or situation is selected from among insects, pests, mold, mildew, fungi, bacteria, an environmental condition or a geographic location.

G36. The method of any one of embodiments G30 to G35, wherein the plant cultivar is selected for root-specific, stem-specific, leaf-specific or flower-specific expression of a terpene synthase, a terpene, a cannabinoid or a flavonoid based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof.

G37. The method of any one of embodiments G1 to G29, further comprising, based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof, genetically modifying a plant cultivar whereby the expression of at least one terpene synthase gene is inhibited or increased in the plant cultivar.

G38. The method of embodiment G37, wherein the genetic modification increases the production of at least one terpene or decreases the production of at least one terpene in the plant cultivar.

G39. The method of embodiment G37 or G38, wherein the plant cultivar is of a Cannabis cultivar.

G40. The method of embodiment G39, wherein the genetic modification increases the production of at least one cannabinoid or decreases the production of at least one cannabinoid in the plant cultivar.

G41. The method of any one of embodiments G37 to G40, wherein the genetic modification is for imparting a medicinal use.

G42. The method of any one of embodiments G37 to G41, wherein the genetic modification is for imparting resistance to an organism or situation.

G43. The method of any one of embodiments G37 to G41, wherein the genetic modification is for imparting affinity towards an organism or situation.

G44. The method of embodiment G42 or G43, wherein the organism or situation is selected from among insects, pests, mold, mildew, fungi, bacteria, an environmental condition or a geographic location.

G45. The method of any one of embodiments G37 to G44, wherein the genetic modification is for imparting root-specific, stem-specific, leaf-specific or flower-specific expression or inhibition of expression of a terpene synthase gene, a terpene, a cannabinoid or a flavonoid.

G46. The method of any one of embodiments G37 to G45, wherein the genetic modification is by a method comprising CRISPR-cas9, Cre-Lox, MiRNA, SiRNA, ShRNA or a combination thereof.

G47. The method of any one of embodiments G1 to G46, wherein the unique subsequence of at least one terpene synthase gene is outside the sequence encoding the active site of the terpene synthase gene.

H1. A method of producing a daughter plant cultivar, comprising:
analyzing two or more parent plant cultivars by the method of any one of embodiments G1 to G29;
based on obtaining the terpene synthase gene profile, determining the expression profile of one or more terpene synthase genes, determining the production profile of one or more terpenes, determining the production profile of one or more cannabinoids, determining the production profile of one or more flavonoids or a combination thereof, selecting two parent plant cultivars for producing a desired daughter plant cultivar; and
inbreeding or outcrossing the parent plant cultivars to produce a daughter plant cultivar.

H2. The method of embodiment H1, wherein the daughter plant cultivar produced has increased expression of at least one terpene synthase gene or decreased expression of at least one terpene synthase gene compared to at least one of the parent plant cultivars.

H3. The method of embodiment H1 or H2, wherein the daughter plant cultivar produced has increased production of at least one terpene or decreased production of at least one terpene compared to at least one of the parent plant cultivars.

H4. The method of any one of embodiments H1 to H3, wherein the parent plant cultivars and the daughter plant cultivar are Cannabis cultivars.

H5. The method of embodiment H4, wherein the daughter plant cultivar produced has increased production of at least one cannabinoid or decreased production of at least one cannabinoid compared to at least one of the parent plant cultivars.

H6. The method of any one of embodiments H1 to H5, wherein the daughter plant cultivar has a medicinal use that is reduced or absent in the parent plant cultivars.

H7. The method of any one of embodiments H1 to H6, wherein the daughter plant cultivar has resistance to an organism or situation, where the resistance is reduced or absent in the parent plant cultivars.

H8. The method of any one of embodiments H1 to H6, wherein the daughter plant cultivar has affinity towards an organism or situation, where the affinity is reduced or absent in the parent plant cultivars.

H9. The method of embodiment H7 or H8, wherein the organism or situation is selected from among insects, pests, mold, mildew, fungi, bacteria, an environmental condition ora geographic location.

H10. The method of any one of embodiments H1 to H9, wherein the daughter plant cultivar has increased root-specific, stem-specific, leaf-specific or flower-specific expression or inhibition of expression of a terpene synthase gene, a terpene, a cannabinoid or a flavonoid compared to at least one of the parent plant cultivars.

I1. A kit, comprising:
one or more single-stranded polynucleotide species, wherein each single-stranded polynucleotide species specifically binds to a unique subsequence of a terpene synthase gene, wherein the unique subsequence of the terpene synthase gene is different than the other subsequences of the terpene synthase gene and the unique subsequence of the terpene synthase gene is different than the subsequences of other terpene synthase genes; and
instructions for use in obtaining a terpene synthase gene profile of a plant cultivar.

I2. The kit of embodiment I1, wherein the single-stranded polynucleotide species specifically binds to a conserved region of the unique subsequence.

I3. The kit of embodiment I1 or I2, wherein the unique subsequence is an exon, an intron, a portion within an exon or a portion within an intron.

I4. The kit of any one of embodiments I1 to I3, wherein the unique subsequence is an exon or a portion within an exon.

I5. The kit of any one of embodiments I1 to I4, wherein the single-stranded polynucleotide species is selected from among one or more of SEQ ID NOS: 1-1284.

I5.0. The kit of any one of embodiments I1 to I4, wherein the single-stranded polynucleotide species is selected from among one or more of SEQ ID NOS: 1328-1338.

I5.01. The kit of any one of embodiments I1 to I4, wherein the single-stranded polynucleotide species is selected from among one or more of SEQ ID NOS: 1285-1327.

I5.02. The kit of any one of embodiments I1 to I4, comprising one or more sets of single-stranded polynucleotide species, wherein the sets are selected from among SEQ ID NOS: 1285-1293; SEQ ID NOS: 1294-1302; SEQ ID NOS: 1303-1311; SEQ ID NOS: 1312-1319; and SEQ ID NOS: 1320-1327.

I5.1 The kit of any one of embodiments I1 to I5.02, wherein the single-stranded polynucleotide species or sets of single-stranded polynucleotide species is selected from among those set forth in SEQ ID NOS: 1-1284, SEQ ID NOS: 1328-1338, SEQ ID NOS: 1285-1327, sets of single-stranded polynucleotide species selected from among SEQ ID NOS: 1285-1293; SEQ ID NOS: 1294-1302; SEQ ID NOS: 1303-1311; SEQ ID NOS: 1312-1319; and SEQ ID NOS: 1320-1327, or from among sequences that share 90%, 91%, 92%, 93%, 94%, 95%, 96%, 97%, 98%, 99% or more identity with any of the sequences set forth in SEQ ID NOS: 1-1284, SEQ ID NOS: 1328-1338, SEQ ID NOS: 1285-1327, and sets of single-stranded polynucleotide species selected from among SEQ ID NOS: 1285-1293; SEQ ID NOS: 1294-1302; SEQ ID NOS: 1303-1311; SEQ ID NOS: 1312-1319; and SEQ ID NOS: 1320-1327.

I6. The kit of any one of embodiments I1 to I5.1, wherein the terpene synthase gene is a monoterpene synthase gene, a diterpene synthase gene or a sesquiterpene synthase gene.

I7. The kit of any one of embodiments I1 to I6, wherein each single-stranded polynucleotide species specifically binds to a unique subsequence of a terpene synthase gene from a Cannabis cultivar.

I8. The kit of any one of embodiments I1 to I7, further comprising an electrical detection label, an electronic detection label, an optical label, such as a chromophore, a dye, or a fluorescent label, or a radiolabel for detecting the specific binding of each single-stranded polynucleotide species to a corresponding unique subsequence of a terpene synthase.

I9. The kit of any one of embodiments I1 to I8, wherein if more than one single-stranded polynucleotide species is present, the single-stranded polynucleotide species bind to different unique subsequences of the same terpene synthase gene, to different unique subsequences of different terpene synthase genes, or to different unique subsequences of the same terpene synthase gene and to different unique subsequences of different terpene synthase genes.

I10. The kit of any one of embodiments I1 to I9, wherein the unique subsequence of at least one terpene synthase gene is outside the sequence encoding the active site of the terpene synthase gene.

J1. A kit, comprising:
one or more polynucleotide primer pairs, wherein each polynucleotide primer pair hybridizes to a unique subsequence of a terpene synthase gene, wherein the unique subsequence of the terpene synthase gene is different than the other subsequences of the terpene synthase gene and the unique subsequence of the terpene synthase gene is different than the subsequences of other terpene synthase genes; and
instructions for use in analyzing the nucleic acid of a plant cultivar.

J2. The kit of embodiment J1, wherein each of the primers of a polynucleotide primer pair hybridizes to a conserved region of the subsequence and the hybridized polynucleotide primer pair flanks a variable region of the subsequence.

J3. The kit of embodiment J1 or J2, wherein the unique subsequence is an exon, an intron, a portion within an exon or a portion within an intron.

J4. The kit of any one of embodiments J1 to J3, wherein the unique subsequence is an exon or a portion within an exon.

J5. The kit of any one of embodiments J1 to J4, wherein each primer pair is selected from among SEQ ID NOS: 1-1284.

J5.1. The kit of any one of embodiments J1 to J5, wherein each primer pair is selected from among those set forth in SEQ ID NOS: 1-1284, or from among sequences that share 90%, 91%, 92%, 93%, 94%, 95%, 96%, 97%, 98%, 99% or more identity with any of the sequences set forth in SEQ ID NOS: 1-1284.

J6. The kit of any one of embodiments J1 to J5.1, wherein the terpene synthase gene is a monoterpene synthase gene, a diterpene synthase paralog or a sesquiterpene synthase paralog.

J7. The kit of any one of embodiments J1 to J6, wherein each polynucleotide primer pair specifically binds to a unique subsequence of a terpene synthase paralog from a Cannabis cultivar.

J8. The kit of any one of embodiments J1 to J8, wherein if more than one polynucleotide primer pair is present, each polynucleotide primer pair binds to different unique subsequences of the same terpene synthase paralog, to different unique subsequences of different terpene synthase paralogs, or to different unique subsequences of the same terpene synthase paralog and to different unique subsequences of different terpene synthase paralogs.

J9. The kit of any one of embodiments J1 to J8, further comprising reagents for amplification of nucleic acid from a plant cultivar.

J10. The kit of any one of embodiments J1 to J9, wherein the unique subsequence of at least one terpene synthase paralog is outside the sequence encoding the active site of the terpene synthase paralog.

K1. A method of preparing primers for uniquely amplifying a gene or a paralog thereof, comprising:
(c) identifying, within at least one exon of the gene or paralog thereof, a first sequence and a second sequence that are conserved for that gene or paralog thereof, wherein the first conserved sequence and the second conserved sequence flank a sequence that is not conserved; and
(d) preparing a pair of primers, wherein:
   (iv) one primer hybridizes to the first conserved sequence and one primer hybridizes to the second conserved sequence;
   (v) each primer, when hybridized to a sequence of the gene or paralog thereof that is not its target conserved sequence, has at least 5 mismatches with the sequence; and
   (vi) each primer, when hybridized to a sequence of the gene or paralog thereof that is not its target conserved sequence, has at least 3 mismatches with the sequence within 5 bases from the 3′-end of the primer.

K2. The method of embodiment K1, wherein the size of the product that is amplified by the prepared pair of primers is 300 base pairs or less.

K3. The method of embodiment K1 or K2, wherein the size of the product that is amplified by the prepared pair of primers is 292 base pairs or less.

K4. The method of any one of embodiments K1 to K3, wherein the size of the product that is amplified by the prepared pair of primers is between about 40 base pairs to about 200 base pairs.

K5. The method of any one of embodiments K1 to K4, wherein the size of the product that is amplified by the prepared pair of primers is between about 50 base pairs to about 150 base pairs.

K6. The method of any one of embodiments K1 to K5, wherein the melting temperature of each primer hybridized to its target conserved sequence is between about 57° C. to about 63° C.

K7. The method of any one of embodiments K1 to K6, wherein the difference between the melting temperatures of each primer of the primer pair hybridized to its target sequence is 3° C. or less.

K8. The method of any one of embodiments K1 to K7, wherein, for at least one exon of the gene or paralog thereof, more than one primer pair is prepared, wherein each primer pair amplifies a difference sequence within the exon of the gene or paralog thereof.

K9. The method of any one of embodiments K1 to K8, wherein more than one primer pair is prepared and at least two primer pairs amplify sequences within two different exons of the gene or paralog thereof.

K10. The method of any one of embodiments K1 to K9, wherein the gene or paralog thereof is of a terpene synthase gene.

L1. A genetically modified plant cultivar produced by the method of any one of embodiments A39 to A48 and C1 to C12.

L2. The genetically modified plant cultivar of embodiment L1, wherein the plant cultivar is a Cannabis cultivar.

M1. A method of identifying whether a plant cultivar comprises a terpene synthase gene or a paralog thereof that has been genetically modified, comprising:
(a) obtaining a nucleic acid sample from the plant cultivar;
(b) contacting the nucleic acid sample with the solid support of any one of embodiments E1 to E11 or the collection of any one of embodiments F1 to F12, thereby preparing a mixture, wherein at least one single-stranded polynucleotide species of the solid support or the collection can specifically bind to at least one genetically modified unique subsequence of a terpene synthase gene or a paralog thereof in the nucleic acid sample, when the at least one genetically modified unique subsequence is present, wherein the at least one genetically modified unique subsequence of the terpene synthase gene or a paralog thereof is different than the other subsequences of the terpene synthase gene or paralog thereof and the unique subsequence of the terpene synthase gene is different than the subsequences of other terpene synthase genes and/or paralogs thereof; and
(c) subjecting the mixture to conditions that facilitate specific binding of the at least one single-stranded polynucleotide species of the solid support or the collection to its corresponding at least one genetically modified unique subsequence of a terpene synthase gene or paralog thereof in the nucleic acid sample, when the at least one genetically modified unique subsequence is present in the nucleic acid sample; and
(d) detecting at least one single-stranded polynucleotide species of the solid support or the collection as being specifically bound to its corresponding genetically modified unique subsequence of a terpene synthase gene or paralog thereof in the nucleic acid sample wherein, if the at least one genetically modified unique subsequence is detected as specifically bound to a solid support or a collection in (c), the plant cultivar is identified as comprising a genetically modified terpene synthase or paralog thereof.

M2. The method of embodiment M1, wherein the detecting is by a signal that is generated when a single-stranded polynucleotide species of the collection binds to its corresponding genetically modified unique subsequence.

M3. The method of embodiment M2, wherein the signal is an electrical signal, an electronic signal, from an optical label or from a radiolabel.

M4. The method of any one of embodiments M1 to M3 further comprising, if the plant cultivar is identified as comprising a genetically modified terpene synthase or paralog thereof, determining the type of genetic modification.

M5. The method of embodiment M4, wherein the type of genetic modification is selected from among deletions, insertions and substitutions.

M6. The method of embodiment M5, wherein the genetic modification comprises at least one substitution.

M7. The method of embodiment M6, wherein the at least one substitution is in a unique subsequence that expresses the active site of the terpene synthase, or a portion thereof.

M8. The method of any one of embodiments M1 to M7, wherein the plant cultivar is of embodiment L1 or L2.

M9. The method of any one of embodiments M1 to M8, wherein the plant cultivar is a Cannabis cultivar.

M10. The method of any one of embodiments M1 to M9, wherein the at least one genetically modified unique subsequence comprises an exon.

N1. A method of breeding one or more plant cultivars, comprising:
(i) obtaining one or more plant cultivars or samples therefrom;
(ii) preparing or analyzing nucleic acid from the one or more plant cultivars according to the method of any one of embodiments A1 to A50, Dl-D2 and Gl-G47;
(iii) based on (ii), identifying one or more plant cultivars as desirable for breeding or as not desirable for breeding; and
(iv) if one or more plant cultivars are identified as desirable for breeding in (iii), breeding all or a subset of the one or more plant cultivars so identified.

N1.1. A method of producing offspring from one or more plant cultivars, comprising:
(i) obtaining one or more plant cultivars or samples therefrom;
(ii) analyzing nucleic acid from the one or more plant cultivars according to the method of any one of embodiments A1 to A50, D1-D2 and G1-G47;
(iii) based on (ii), identifying one or more plant cultivars as desirable for breeding or as not desirable for breeding; and
(iv) if one or more plant cultivars are identified as desirable for breeding in (iii), breeding all or a subset of the one or more plant cultivars so identified, thereby producing offspring of the one or more plant cultivars so identified.

N2. The method of embodiment N1 or N1.1, wherein the breeding characteristic identified in (iii) is resistance to an organism or situation or favoring an organism or situation.

N3. The method of embodiment N2, wherein the organism or situation is selected from among insects, pests, mold, chemicals, mildew, fungi, bacteria, viruses, an environmental condition or a geographic location.

N4. The method of any one of embodiments N1 to N3, further comprising, in (iii), based on (ii), identifying the terpene abundance profile, the flavonoid profile, the cannabinoid profile, the heredity or a combination thereof of the one or more plant cultivars as desirable for breeding or as not desirable for breeding.

N4.1. The method of embodiment N3 or N4, wherein the plant cultivar is selected for resistance to an organism or situation that is identified based on identifying one or more terpene synthase genes, determining the expression profile of one or more terpene synthase genes, and/or determining the production profile of one or more terpenes.

N4.2. The method of any one of embodiments N3 to N4.1, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting resistance to an organism or situation to a plant cultivar.

N4.3. The method of any one of embodiments N3 to N4.2, wherein the one or more TPS genes, or sets thereof, produce one or more of the terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

N4.4. The method of any one of embodiments N3 to N4.3 that is a method of breeding to produce one or more offspring cultivars that show increased anti-pathogenic properties compared to at least one of the parent cultivars.

N4.5. The method of embodiment N4.4, wherein the one or more offspring cultivars comprises a terpene synthase profile comprising one or more of increased amounts/expression of TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37JL.

N4.6. The method of any one of embodiments N3 to N4.5 that is a method of breeding to produce one or more offspring cultivars comprising one or more root specifically expressed terpene synthases that increase resistance against pests in the soil and/or one or more root specifically expressed terpene synthases that respond favorably to beneficial microorganisms in the soil, compared to at least one of the parent cultivars.

N4.7. The method of embodiment N4.6, wherein the one or more offspring cultivars comprises a terpene synthase profile comprising one or more of increased amounts/expression of TPS11JL, TPS49JL, TPS41JL, TPS12JL, TPS11-likeJL, TPS36JL, TPS6JL, TPS37JL and TPS64JL.

N4.8. The method of any one of embodiments N3 to N4.7 that is a method of breeding to produce one or more offspring cultivars comprising one or more stem specifically expressed terpene synthases that increase resistance against stem-hosted pests, compared to at least one of the parent cultivars.

N4.9. The method of embodiment N4.8, wherein the one or more offspring cultivars comprises a terpene synthase profile comprising one or more of increased amounts/expression of TPS63JL, TPS43JL, TPS41JL, TPS6-likeJL, TPS33JL and TPS24JL.

N4.10. The method of any one of embodiments N3 to N4.9 that is a method of breeding to produce one or more offspring cultivars comprising one or more herbicidal properties, compared to at least one of the parent cultivars.

N4.11. The method of embodiment N4.10, wherein the one or more offspring cultivars comprises a terpene profile comprising one or more of increased amounts of geraniol, pilegone, citronellol, borneol and para-cymene.

N4.12. The method of any one of embodiments N3 to N4.11 that is a method of breeding to produce one or more offspring cultivars comprising that comprise one or more pesticidal properties, compared to at least one of the parent cultivars.

N4.13. The method of embodiment N4.12, wherein the one or more offspring cultivars comprises a terpene profile comprising one or more of increased amounts of aromadendrene, α-bisabolol, cedrol, nerolidol, trans-nerolidol and guaiol.

N4.14. The method of embodiment N1 or N1.1, wherein the breeding characteristic identified in (iii) is affinity towards an organism or situation or favoring an organism or situation.

N4.15. The method of embodiment N4.14, wherein the plant cultivar is selected for having an affinity towards an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof.

N4.16. The method of embodiment N4.14 or N4.15 that is a method of breeding to produce one or more offspring cultivars comprising one or more insect pheromonal properties, compared to at least one of the parent cultivars.

N4.17. The method of embodiment N4.16, wherein the one or more offspring cultivars comprises a terpene profile comprising one or more of increased amounts of endo-borneol, isoborneol, 3-carene, carveol, germacrene B, hedycaryol, menthol, cis-nerolidol, cis-β-ocimene, trans-β-ocimene, sabinene hydrate, α-terpinene, thymol, β-farnesene, α-farnesene, γ-eudesmol, alloaromadendrene, valencene and pulegone.

N4.18. The method of any one of embodiments N4.14 to N4.17, wherein the organism or situation is selected from among exposure to insects, pests, mold, chemicals, mildew, fungi, bacteria, an environmental condition or a geographic location.

N5. The method of any one of embodiments N1 to N4.18, further comprising, in (iii), based on (ii), identifying one or more therapeutic activities of the one or more plant cultivars as desirable for breeding or as not desirable for breeding.

N6. The method of embodiment N5, wherein the one or more therapeutic activities are selected from among antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

N7. The method of embodiment N5 or N6, wherein the plant cultivar is selected for a therapeutic activity that is assigned based on identifying one or more terpene synthase genes, determining the expression profile of one or more terpene synthase genes, and/or determining the production profile of one or more terpenes.

N7.1. The method of embodiment N7, wherein the therapeutic activity is to impart energy, mental clarity, appetite stimulation or appetite suppression.

N7.2. The method ofembodiment N7 or embodiment N7.1, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting one or more therapeutic activities to a plant cultivar.

N7.3. The method of any one of embodiments N7 to N7.2, wherein the one or more TPS genes, or sets thereof, produce one or more of the terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, −-Cubebene, Linalyl acetate, Bornyl acetate, Heptacosane, Tricosane, S-Limonene, (−)-Thujopsene, Hashenene 5,5-dimethyl-1-vinylbicyclo[2.1.1]hexane, (−)-englerin A and Artemisinin.

N7.4. The method of any one of embodiments N1 to N7.4 that is a method of breeding to produce one or more offspring cultivars that show increased cannabinoid production compared to at least one of the parent cultivars.

N7.5. The method of embodiment N7.4, wherein the one or more offspring cultivars show reduced expression, or lack of expression, of one or more terpene synthases selected from among TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL and TPS37JL compared to at least one of the parent cultivars.

N7.6. The method of any one of embodiments N1 to N7.6 that is a method of breeding to produce one or more offspring cultivars that produces an increased energetic effect compared to at least one of the parent cultivars.

N7.7. The method of embodiment N7.6, wherein the one or more offspring cultivars comprises a terpene profile comprising one or more of increased S-linalool production, increased terpinolene production, increased β-ocimene production, α-pinene production greater than p-pinene production, reduced or lack of R-linalool production, reduced or lack of α-terpineol production and reduced or lack of fenchol production compared to at least one of the parent cultivars.

N7.8. The method of any one of embodiments N1 to N7.7 that is a method of breeding to produce one or more offspring cultivars that produces an increased sedative effect compared to at least one of the parent cultivars.

N7.9. The method of embodiment N7.8, wherein the one or more offspring cultivars comprises a terpene profile comprising one or more of: about equal or equal amounts of p-pinene and α-pinene production, increased R-linalool production, increased limonene production, increased trans-nerolidol production, increased terpineol production, increased camphene production, reduced or lack of β-ocimene production, reduced or lack of S-linalool production and reduced or lack of terpinolene production compared to at least one of the parent cultivars.

N7.10. The method of any one of embodiments N1 to N7.9 that is a method of breeding to produce one or more offspring cultivars that produces an increased cognitive-enhancing effect compared to at least one of the parent cultivars.

N7.11. The method of embodiment N7.10, wherein the one or more offspring cultivars comprises a terpene profile comprising one or more of: greater amounts of β-pinene production relative to α-pinene production, increased β-ocimene production and increased eucalyptol production compared to at least one of the parent cultivars.

N7.12. The method of any one of embodiments N1 to N7.11 that is a method of breeding to produce one or more offspring cultivars that produces an increased appetite-suppressing effect compared to at least one of the parent cultivars.

N7.13. The method of embodiment N7.12, wherein the one or more offspring cultivars comprises a terpene profile comprising increased amounts of humulene production compared to at least one of the parent cultivars.

N7.14. The method of any one of embodiments N1 to N7.13 that is a method of breeding to produce one or more offspring cultivars that produces an increased anti-inflammatory effect compared to at least one of the parent cultivars.

N7.15. The method of embodiment N7.14, wherein the one or more offspring cultivars comprises a terpene profile comprising one or more of: increased α-pinene production, increased humulene production and increased β-caryophyllene production compared to at least one of the parent cultivars.

N7.16. The method of any one of embodiments N1 to N7.15 that is a method of breeding to produce one or more offspring cultivars that produces an increased anti-anxiety effect compared to at least one of the parent cultivars.

N7.17. The method of embodiment N7.16, wherein the one or more offspring cultivars comprises a terpene profile comprising one or more of: increased β-pinene production, increased humulene production, increased β-caryophyllene production, increased linalool production, increased nerolidol production and increased limonene production compared to at least one of the parent cultivars.

N7.18. The method of any one of embodiments N1 to N7.17 that is a method of breeding to produce one or more offspring cultivars that produces an increased antinociceptive effect compared to at least one of the parent cultivars.

N7.19. The method of embodiment N7.18, wherein the one or more offspring cultivars comprises a terpene profile comprising one or more of: increased α-bisabolol production, increased α-terpineol production, increased trans nerolidol production, increased α-phellandrene production, and increased eucalyptol production compared to at least one of the parent cultivars.

N7.20. The method of any one of embodiments N1 to N7.19 that is a method of breeding to produce one or more offspring cultivars that produces an increased body relaxing effect compared to at least one of the parent cultivars.

N7.21. The method of embodiment N7.20, wherein the one or more offspring cultivars comprises a terpene profile comprising one or more of: increased α-bisabolol production, increased α-terpineol production, increased trans nerolidol production and increased α-phellandrene production, compared to at least one of the parent cultivars.

N7.22. The method of any one of embodiments N1 to N7.21 that is a method of breeding to produce one or more offspring cultivars that produces an increased anti-depressant effect compared to at least one of the parent cultivars.

N7.23. The method of embodiment N7.22, wherein the one or more offspring cultivars comprises a terpene profile comprising one or more of: equal or about equal amounts of α-pinene and β-pinene production, increased limonene production, increased nerolidol production and increased linalool production, compared to at least one of the parent cultivars.

N7.24. The method of any one of embodiments N1 to N7.23 that is a method of breeding to produce one or more offspring cultivars that produces an increased amount of one or more acetyl cholinesterase-inhibitor (AChEI) terpenes compared to at least one of the parent cultivars.

N7.25. The method of embodiment N7.24, wherein the one or more offspring cultivars comprises a terpene profile comprising one or more of: increased amounts of α-pinene production, increased terpinolene production, increased β-ocimene production, increased 3-carene production, increased α and/or γ-terpinene production and increased sabinene production compared to at least one of the parent cultivars.

N7.26. The method of any one of embodiments N1 to N7.25 that is a method of breeding to produce one or more offspring cultivars that produces an increased anti-bacterial effect compared to at least one of the parent cultivars.

N7.27. The method of embodiment N7.26, wherein the one or more offspring cultivars comprises a terpene profile comprising one or more of: increased amounts of aromadendrene production, increased carvacrol production, increased β-caryophyllene production, increased eucalyptol production, increased fenchol production, increased germacrene D production, increased nerol production, increased pulegone production, increased sabinene production and increased geraniol production compared to at least one of the parent cultivars.

N7.28. The method of any one of embodiments N1 to N7.27 that is a method of breeding to produce one or more offspring cultivars that produces an increased anti-microbial effect compared to at least one of the parent cultivars.

N7.29. The method of embodiment N7.28, wherein the one or more offspring cultivars comprises a terpene profile comprising one or more of: increased amounts of camphor production, increased sabinene hydrate production and increased thymol production compared to at least one of the parent cultivars.

N7.30. The method of any one of embodiments N1 to N7.29 that is a method of breeding to produce one or more offspring cultivars that produces an increased fungicidal effect compared to at least one of the parent cultivars.

N7.31. The method of embodiment N7.30, wherein the one or more offspring cultivars comprises a terpene profile comprising one or more of: increased amounts of citronellol production, increased para-cymene production, increased pulegone production and increased geraniol production compared to at least one of the parent cultivars.

N7.32. The method of any one of embodiments N1 to N7.31 that is a method of breeding to produce one or more offspring cultivars that produces an increased expectorant effect compared to at least one of the parent cultivars.

N7.33. The method of embodiment N7.32, wherein the one or more offspring cultivars comprises a terpene profile comprising one or more of: increased amounts of camphene production, increased sabinene hydrate production and increased geraniol production compared to at least one of the parent cultivars.

N7.34. The method of any one of embodiments N1 to N7.33 that is a method of breeding to produce one or more offspring cultivars that produces an increased expectorant effect compared to at least one of the parent cultivars.

N7.35. The method of embodiment N7.34, wherein the one or more offspring cultivars comprises a terpene profile comprising one or more of: increased amounts of camphene production, increased sabinene hydrate production and increased geraniol production compared to at least one of the parent cultivars.

N7.36. The method of any one of embodiments N1 to N7.35 that is a method of breeding to produce one or more offspring cultivars that produces increased non-irritant properties compared to at least one of the parent cultivars.

N7.37. The method of embodiment N7.36, wherein the one or more offspring cultivars comprises a terpene profile comprising one or more of reduced or absent: borneol, α-cedrene, citronellol or para-cymene, or increased amounts of the counter-irritant fenchone.

N8. The method of any one of embodiments Ni to N7.37, wherein the one or more plant cultivars are Cannabis cultivars.

O1. A method of cultivating one or more plant cultivars as a crop, comprising:
(i) obtaining one or more plant cultivars or samples therefrom;
(ii) preparing or analyzing nucleic acid from the one or more plant cultivars according to the method of any one of embodiments A1 to A50, D1-D2 and G1-G47;
(iii) based on (ii), identifying one or more plant cultivars as desirable for cultivating as a crop or as not desirable for cultivating as a crop; and
(iv) if one or more plant cultivars are identified as desirable for cultivating as a crop in (iii), cultivating all or a subset of the one or more plant cultivars so identified.

O2. The method of embodiment O1, wherein the cultivating characteristic identified in (iii) is resistance to an organism or situation or favoring an organism or situation.

O3. The method of embodiment O2, wherein the organism or situation is selected from among insects, pests, mold, chemicals, mildew, fungi, bacteria, viruses, an environmental condition or a geographic location.

O4. The method of any one of embodiments O1 to O3, further comprising, in (iii), based on (ii), identifying the terpene abundance profile, the flavonoid profile, the cannabinoid profile, the heredity or a combination thereof of the one or more plant cultivars as desirable for cultivating or as not desirable for cultivating.

O5. The method of any one of embodiments O1 to O4, further comprising, in (iii), based on (ii), identifying one or more the therapeutic activities of the one or more plant cultivars as desirable for cultivating or as not desirable for cultivating.

O6. The method of embodiment N5, wherein the one or more therapeutic activities are selected from among antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

O7. The method of any one of embodiments O1 to O6, wherein the one or more plant cultivars are Cannabis cultivars.

P1. A method of treating a subject with one or more plant cultivars or a portion thereof or an extract thereof, comprising:
(i) obtaining one or more plant cultivars or samples therefrom;
(ii) preparing or analyzing nucleic acid from the one or more plant cultivars according to the method of any one of embodiments A1 to A50, D1-D2 and G1-G47;
(iii) based on (ii), identifying one or more plant cultivars as desirable for treating a subject or as not desirable for treating a subject; and
(iv) if one or more plant cultivars are identified as desirable for treating a subject in (iii), treating the subject with the one or more plant cultivars identified according to (iii), or with a portion thereof, or with an extract thereof.

P2. The method of embodiment P1, wherein the treatment characteristic identified in (iii) is antioxidant, anti-inflammatory, antibacterial, antiviral, anti-anxiety, antinociceptive, analgesic, antihypertensive, sedative, antidepressant, acetylcholine esterase inhibition (AChEI), neuro-protective or gastro-protective effects, or any combination thereof.

P3. The method of embodiment P1 or P2, wherein the one or more plant cultivars are Cannabis cultivars.

P4. The method of any one of embodiments P1 to P3, wherein the subject is a human or an animal.

P5. The method of any one of embodiments P1 to P4, wherein the portion thereof is a seed, flower, stem or leaf of the one or more plant cultivars.

P6. The method of any one of embodiments P1 to P5, wherein the subject is treated with a portion or an extract of the one or more plant cultivars.

P7. The method of any one of embodiments P1 to P6, wherein the treatment is administered orally, topically, or through inhalation.

P8. The method of any one of embodiments P1 to P7, wherein the treatment is self-administered, or is administered by an entity other than the subject.

Q1. A method of breeding one or more plant cultivars, comprising:
(i) obtaining one or more plant cultivars or samples therefrom;
(ii) identifying whether the one or more plant cultivars comprise(s) a terpene synthase gene or a paralog thereof that has been genetically modified according to the method of any one of embodiments M1 to M10;
(iii) based on (ii), identifying one or more plant cultivars as desirable for breeding or as not desirable for breeding; and
(iv) if one or more plant cultivars are identified as desirable for breeding in (iii), breeding all or a subset of the one or more plant cultivars so identified.

Q2. The method of embodiment Q1, wherein the breeding characteristic identified in (iii) is resistance to an organism or situation or favoring an organism or situation.

Q3. The method of embodiment Q2, wherein the organism or situation is selected from among insects, pests, mold, chemicals, mildew, fungi, bacteria, viruses, an environmental condition or a geographic location.

Q4. The method of any one of embodiments Q1 to Q3, further comprising, in (iii), based on (ii), identifying the terpene abundance profile, the flavonoid profile, the cannabinoid profile, the heredity or a combination thereof of the one or more plant cultivars as desirable for breeding or as not desirable for breeding.

Q5. The method of any one of embodiments Q1 to Q4, further comprising, in (iii), based on (ii), identifying one or more the therapeutic activities of the one or more plant cultivars as desirable for breeding or as not desirable for breeding.

Q6. The method of embodiment Q5, wherein the one or more therapeutic activities are selected from among antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

Q7. The method of any one of embodiments Q1 to Q6, wherein the one or more plant cultivars are Cannabis cultivars.

R1. A method of cultivating one or more plant cultivars as a crop, comprising:
(i) obtaining one or more plant cultivars or samples therefrom;
(ii) identifying whether the one or more plant cultivars comprise(s) a terpene synthase gene or a paralog thereof that has been genetically modified according to the method of any one of embodiments M1 to M10;
(iii) based on (ii), identifying one or more plant cultivars as desirable for cultivating as a crop or as not desirable for cultivating as a crop; and
(iv) if one or more plant cultivars are identified as desirable for cultivating as a crop in (iii), cultivating all or a subset of the one or more plant cultivars so identified.

R2. The method of embodiment R1, wherein the cultivating characteristic identified in (iii) is resistance to an organism or situation or favoring an organism or situation.

R3. The method of embodiment R2, wherein the organism or situation is selected from among insects, pests, mold, chemicals, mildew, fungi, bacteria, viruses, an environmental condition or a geographic location.

R4. The method of any one of embodiments R1 to R3, further comprising, in (iii), based on (ii), identifying the terpene abundance profile, the flavonoid profile, the cannabinoid profile, the heredity or a combination thereof of the one or more plant cultivars as desirable for cultivating or as not desirable for cultivating.

R5. The method of any one of embodiments R1 to R4, further comprising, in (iii), based on (ii), identifying one or more the therapeutic activities of the one or more plant cultivars as desirable for cultivating or as not desirable for cultivating.

R6. The method of embodiment R5, wherein the one or more therapeutic activities are selected from among antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

R7. The method of any one of embodiments R1 to R6, wherein the one or more plant cultivars are Cannabis cultivars.

S1. A method of treating a subject with one or more plant cultivars or a portion thereof or an extract thereof, comprising:
obtaining one or more plant cultivars or samples therefrom;
(ii) identifying whether the one or more plant cultivars comprise(s) a terpene synthase gene or a paralog thereof that has been genetically modified according to the method of any one of embodiments M1 to M10;
(iii) based on (ii), identifying one or more plant cultivars as desirable for treating a subject or as not desirable for treating a subject; and
(iv) if one or more plant cultivars are identified as desirable for treating a subject in (iii), treating the subject with the one or more plant cultivars identified according to (iii), or with a portion thereof, or with an extract thereof.

S2. The method of embodiment S1, wherein the treatment characteristic identified in (iii) is antioxidant, anti-inflammatory, antibacterial, antiviral, anti-anxiety, antinociceptive, analgesic, antihypertensive, sedative, antidepressant, acetylcholine esterase inhibition (AChEI), neuro-protective or gastro-protective effects, or any combination thereof.

S3. The method of embodiment S1 or S2, wherein the one or more plant cultivars are Cannabis cultivars.

S4. The method of any one of embodiments S1 to S3, wherein the subject is a human or an animal.

S5. The method of any one of embodiments S1 to S4, wherein the portion thereof is a seed, flower, stem or leaf of the one or more plant cultivars.

S6. The method of any one of embodiments S1 to S5, wherein the subject is treated with a portion or an extract of the one or more plant cultivars.

S7. The method of any one of embodiments S1 to S6, wherein the treatment is administered orally, topically, or through inhalation.

S8. The method of any one of embodiments S1 to S7, wherein the treatment is self-administered, or is administered by an entity other than the subject.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications can be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method of analyzing a plant cultivar comprising at least one terpene synthase gene or a paralog thereof, the method comprising:
(a) obtaining a nucleic acid sample from the plant cultivar;
(b) contacting the nucleic acid sample with at least one polynucleotide primer pair under amplification conditions, thereby preparing a mixture, wherein the polynucleotide primer pair hybridizes to a unique subsequence of the terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene or a paralog thereof is different than the subsequences of other terpene synthase genes and/or paralogs thereof;
(c) amplifying the mixture, thereby obtaining an amplified mixture; and
(d) analyzing the amplified mixture of (c), whereby at least one terpene synthase gene or a paralog thereof is identified and/or quantified in the amplified mixture.

2. The method of claim 1, wherein the plant cultivar comprises a plurality of terpene synthase genes and/or paralogs thereof and the method comprises:
in (b), contacting the nucleic acid sample with a plurality of polynucleotide primer pairs under amplification conditions, thereby preparing a mixture, wherein each of the plurality of polynucleotide primer pairs hybridizes to a unique subsequence of a terpene synthase gene or a paralog thereof, wherein the unique subsequence of the terpene synthase gene or a paralog thereof is different than the other subsequences of the terpene synthase gene or a paralog thereof and the unique subsequence of the terpene synthase gene or a paralog thereof is different than the subsequences of the other terpene synthase genes and/or paralogs thereof; and
in (d), a plurality of terpene synthase genes and/or paralogs thereof are identified and/or quantified.

3. The method of claim 1, wherein the unique subsequence is an exon or a portion within an exon.

4. The method of claim 1, wherein the identification in (d) is by one or more of high-resolution melting (HRM), quantitative PCR (qPCR), loop-mediated isothermal amplification (LAMP), restriction endonuclease digestion, gel electrophoresis and sequencing.

5. The method of claim 1, wherein the at least one polynucleotide primer pair is selected from among those set forth in SEQ ID NOS: 1-1284.

6. The method of claim 2, wherein the plurality of primer pairs are selected from among those set forth in SEQ ID NOS: 1-1284.

7. The method of claim 1, wherein the identification and/or quantification in (d) is by loop-mediated isothermal amplification (LAMP).

8. The method of claim 7, wherein a gene for the production of terpinolene is identified and/or quantified in (d).

9. The method of claim 8, wherein the polynucleotide primer pairs are present in a set of primers selected from among SEQ ID NOS:1285-1293, SEQ ID NOS:1294-1302, SEQ ID NOS:1303-1311, SEQ ID NOS:1312-1319 and SEQ ID NOS:1320-1327.

10. The method of claim 1, wherein, based on identifying and/or quantifying at least one terpene synthase gene or a paralog thereof in (d), the terpene synthase gene profile, the terpene synthase expression profile, the terpene production profile, the cannabinoid production profile, the flavonoid production profile, or any combination thereof is determined in the plant cultivar.

11. The method of claim 1, wherein one or more plant cultivars is/are a Cannabis cultivar.

12. The method of claim 1, wherein at least one plant cultivar that is analyzed expresses one or more terpene synthases selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPSSJL, TPSSJL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

13. The method of claim 10, further comprising, based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or any combination thereof, selecting a plant cultivar for breeding offspring.

14. The method of claim 13, wherein the plant cultivar is selected for a medicinal use that is assigned based on identifying one or more terpene synthase genes, determining the expression profile of one or more terpene synthase genes, and/or determining the production profile of one or more terpenes.

15. The method of claim 14, wherein the medicinal use is selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects.

16. The method of claim 14, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting one or more medicinal uses to a plant cultivar.

17. The method of claim 13, wherein the plant cultivar is selected for resistance to an organism or situation that is identified based on identifying one or more terpene synthase genes, determining the expression profile of one or more terpene synthase genes, and/or determining the production profile of one or more terpenes.

18. The method of claim 17, wherein sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes are assigned as imparting resistance to an organism or situation to a plant cultivar.

19. The method of claim 17, wherein the organism or situation is selected from among exposure to insects, insecticides, mold, pesticides, mildew, fungi, bacteria, an environmental condition or a geographic location.

20. The method of claim 19, wherein the organism or situation is exposure to insects and/or exposure to pesticides.

21. A method of producing offspring from one or more plant cultivars, comprising:
(i) obtaining one or more plant cultivars or samples therefrom;
(ii) analyzing nucleic acid from the one or more plant cultivars according to the method of claim 1;
(iii) based on (ii), identifying one or more plant cultivars as desirable for breeding or as not desirable for breeding; and
(iv) if one or more plant cultivars are identified as desirable for breeding in (iii), breeding all or a subset of the one or more plant cultivars so identified, thereby producing offspring of the one or more plant cultivars so identified.

22. The method of claim 21, wherein the one or more plant cultivars are Cannabis cultivars.

23. The method of claim 21 that is a method of producing one or more offspring cultivars that show increased cannabinoid production compared to at least one of the parent cultivars.

24. The method of claim 23, wherein the one or more offspring cultivars show reduced expression, or lack of expression, of one or more terpene synthases selected from among TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPS5JL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL and TPS37JL compared to at least one of the parent cultivars.

25. The method of any one of claim 21 that is a method of producing one or more offspring cultivars that produces an increased sedative effect compared to at least one of the parent cultivars.

26. The method of claim 25, wherein the one or more offspring cultivars comprises a terpene profile comprising one or more of: about equal or equal amounts of β-pinene and α-pinene production, increased R-linalool production, increased limonene production, increased trans-nerolidol production, increased terpineol production, increased camphene production, reduced or lack of β-ocimene production, reduced or lack of S-linalool production and reduced or lack of terpinolene production compared to at least one of the parent cultivars.

27. The method of claim 21 that is a method of producing one or more offspring cultivars that show increased anti-pathogenic properties compared to at least one of the parent cultivars.

28. The method of claim 27, wherein the one or more offspring cultivars comprises a terpene synthase profile comprising one or more of increased amounts/expression of TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPSSJL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37J L.

29. A kit, comprising:
one or more single-stranded polynucleotide species, wherein each single-stranded polynucleotide species specifically binds to a unique subsequence of a terpene synthase gene, wherein the unique subsequence of the terpene synthase gene is different than the other subsequences of the terpene synthase gene and the unique subsequence of the terpene synthase gene is different than the subsequences of other terpene synthase genes; and
instructions for use in obtaining a terpene synthase gene profile of a plant cultivar.

30. The kit of claim 29, wherein the unique subsequence is an exon or a portion within an exon.


SEQUENCE LISTING (1400 sequences)

SEQ ID NO: 1 - DNA, 23 bp, Artificial Sequence
  Description of Artificial Sequence Synthetic primer
atattcaaagatcaaccagcagc

SEQ ID NO: 2 - DNA, 22 bp, Artificial Sequence
  Description of Artificial Sequence Synthetic primer
tgtgggtttgtagttgcctgat

SEQ ID NO: 3 - DNA, 25 bp, Artificial Sequence
  Description of Artificial Sequence Synthetic primer
agctatgggaaaagaatcaatgagc

SEQ ID NO: 4 - DNA, 23 bp, Artificial Sequence
  Description of Artificial Sequence Synthetic primer
gagtttgaaatgaagagcagtgg

SEQ ID NO: 5 - DNA, 25 bp, Artificial Sequence
  Description of Artificial Sequence Synthetic primer
gaggctgaaaatcctttagttaagc

SEQ ID NO: 6 - DNA, 27 bp, Artificial Sequence
  Description of Artificial Sequence Synthetic primer
acaggactgaatccatattgtctaagg

SEQ ID NO: 7 - DNA, 25 bp, Artificial Sequence
  Description of Artificial Sequence Synthetic primer
agatgaagcaagagatttcacaacc

SEQ ID NO: 8 - DNA, 22 bp, Artificial Sequence
  Description of Artificial Sequence Synthetic primer
aatggagtggaagatccaaggc

SEQ ID NO: 9 - DNA, 21 bp, Artificial Sequence
  Description of Artificial Sequence Synthetic primer
atgccttggatcttccactcc

SEQ ID NO: 10 - DNA, 23 bp, Artificial Sequence
  Description of Artificial Sequence Synthetic primer
ctttttggtatgctgactgaacg

SEQ ID NO: 11 - DNA, 24 bp, Artificial Sequence
  Description of Artificial Sequence Synthetic primer
catttgctagagacagagtagtgg

SEQ ID NO: 12 - DNA, 22 bp, Artificial Sequence
  Description of Artificial Sequence Synthetic primer
gctgaagctcatctagtgtacc

SEQ ID NO: 13 - DNA, 20 bp, Artificial Sequence
  Description of Artificial Sequence Synthetic primer
tgaactggatcagctacccg

SEQ ID NO: 14 - DNA, 24 bp, Artificial Sequence
  Description of Artificial Sequence Synthetic primer
gtgtgaatcccattttctttgagg

SEQ ID NO: 15 - DNA, 22 bp, Artificial Sequence
  Description of Artificial Sequence Synthetic primer
gttgggtgatctgtgtaaatgc

SEQ ID NO: 16 - DNA, 22 bp, Artificial Sequence
  Description of Artificial Sequence Synthetic primer
ctgagacacgtaaaatggttgg

SEQ ID NO: 17 - DNA, 20 bp, Artificial Sequence
  Description of Artificial Sequence Synthetic primer
tgctacatgcgtgaaaaggg

SEQ ID NO: 18 - DNA, 22 bp, Artificial Sequence
  Description of Artificial Sequence Synthetic primer
tgtggtacatctccattgctcc

SEQ ID NO: 19 - DNA, 22 bp, Artificial Sequence
  Description of Artificial Sequence Synthetic primer
atgtgctgtggtcaatagttct

SEQ ID NO: 20 - DNA, 21 bp, Artificial Sequence
  Description of Artificial Sequence Synthetic primer
aaagaccaaatggagggctca

SEQ ID NO: 21 - DNA, 22 bp, Artificial Sequence
  Description of Artificial Sequence Synthetic primer
acaggtcgagtcaaagaattgg

SEQ ID NO: 22 - DNA, 20 bp, Artificial Sequence
  Description of Artificial Sequence Synthetic primer
gccatgttgacgtagaagcc

SEQ ID NO: 23 - DNA, 26 bp, Artificial Sequence
  Description of Artificial Sequence Synthetic primer
aggaagagataggaaaaatcaaagcg

SEQ ID NO: 24 - DNA, 20 bp, Artificial Sequence
  Description of Artificial Sequence Synthetic primer
gcgtcgatgaaccacttagc

SEQ ID NO: 25 - DNA, 27 bp, Artificial Sequence
  Description of Artificial Sequence Synthetic primer
taaggaagagataggaaaaatcaaagc

SEQ ID NO: 26 - DNA, 22 bp, Artificial Sequence
  Description of Artificial Sequence Synthetic primer
ttgtagtcctccgatgaagtgg

SEQ ID NO: 27 - DNA, 22 bp, Artificial Sequence
  Description of Artificial Sequence Synthetic primer
tggaggcatactaaacttgggg

SEQ ID NO: 28 - DNA, 24 bp, Artificial Sequence
  Description of Artificial Sequence Synthetic primer
aagctctaattcatccaatgttcc

SEQ ID NO: 29 - DNA, 27 bp, Artificial Sequence
  Description of Artificial Sequence Synthetic primer
gatgggatatggaaatgataaatgagt

SEQ ID NO: 30 - DNA, 23 bp, Artificial Sequence
  Description of Artificial Sequence Synthetic primer
tgttgatggagatgtgttggtct

SEQ ID NO: 31 - DNA, 20 bp, Artificial Sequence
  Description of Artificial Sequence Synthetic primer
ggatttcagtgggagcaccg

SEQ ID NO: 32 - DNA, 22 bp, Artificial Sequence
  Description of Artificial Sequence Synthetic primer
gcactatgacgaattatggcgg

SEQ ID NO: 33 - DNA, 20 bp, Artificial Sequence
  Description of Artificial Sequence Synthetic primer
tgaaaagaggtgatgctccg

SEQ ID NO: 34 - DNA, 24 bp, Artificial Sequence
  Description of Artificial Sequence Synthetic primer
ccatgctgatacatgaaaaatccc

SEQ ID NO: 35 - DNA, 29 bp, Artificial Sequence
  Description of Artificial Sequence Synthetic primer
atccttaatactacaaaattagcaagagc

SEQ ID NO: 36 - DNA, 27 bp, Artificial Sequence
  Description of Artificial Sequence Synthetic primer
aatgtaatcaaaagaccaaatgggagg

SEQ ID NO: 37 - DNA, 23 bp, Artificial Sequence
  Description of Artificial Sequence Synthetic primer
agtagattgaatgagctagaggc

SEQ ID NO: 38 - DNA, 18 bp, Artificial Sequence
  Description of Artificial Sequence Synthetic primer
aattcgagagctgtggcg

SEQ ID NO: 39 - DNA, 22 bp, Artificial Sequence
  Description of Artificial Sequence Synthetic primer
aagcaagttcaaggcaagaacg

SEQ ID NO: 40 - DNA, 22 bp, Artificial Sequence
  Description of Artificial Sequence Synthetic primer
gatggtttgtttcgctccatcg

SEQ ID NO: 41 - DNA, 23 bp, Artificial Sequence
  Description of Artificial Sequence Synthetic primer
ggagtgtttcttatggactgtgg

SEQ ID NO: 42 - DNA, 27 bp, Artificial Sequence
  Description of Artificial Sequence Synthetic primer
gaaaagctctaattcctccaatgttcc

SEQ ID NO: 43 - DNA, 24 bp, Artificial Sequence
  Description of Artificial Sequence Synthetic primer
tgtgaaagcaatggatgatttacc

SEQ ID NO: 44 - DNA, 23 bp, Artificial Sequence
  Description of Artificial Sequence Synthetic primer
aattttggtgtcctaacacatcg

SEQ ID NO: 45 - DNA, 25 bp, Artificial Sequence
  Description of Artificial Sequence Synthetic primer
tctgtaaacatcaattacaagaggc

SEQ ID NO: 46 - DNA, 21 bp, Artificial Sequence
  Description of Artificial Sequence Synthetic primer
tcggtcctgataccgaaatcc

SEQ ID NO: 47 - DNA, 22 bp, Artificial Sequence
  Description of Artificial Sequence Synthetic primer
atggatttcggtatcaggaccg

SEQ ID NO: 48 - DNA, 24 bp, Artificial Sequence
  Description of Artificial Sequence Synthetic primer
catctgtaagtcgtagaatcatgg

SEQ ID NO: 49 - DNA, 27 bp, Artificial Sequence
  Description of Artificial Sequence Synthetic primer
taatgaagatgaaaatatggactctcc

SEQ ID NO: 50 - DNA, 26 bp, Artificial Sequence
  Description of Artificial Sequence Synthetic primer
gataaactatcctgagaagcatgtcc

SEQ ID NO: 51 - DNA, 23 bp, Artificial Sequence
  Description of Artificial Sequence Synthetic primer
tgtccactcaaatcttagcatca

SEQ ID NO: 52 - DNA, 24 bp, Artificial Sequence
  Description of Artificial Sequence Synthetic primer
tgaaatgtttttgtaggacgaaca

SEQ ID NO: 53 - DNA, 24 bp, Artificial Sequence
  Description of Artificial Sequence Synthetic primer
caacccaaataaaattgttcgtcc

SEQ ID NO: 54 - DNA, 27 bp, Artificial Sequence
  Description of Artificial Sequence Synthetic primer
ttgtgaaatgttgtaatgtaagaatcg

SEQ ID NO: 55 - DNA, 22 bp, Artificial Sequence
  Description of Artificial Sequence Synthetic primer
tgtcaacaacaaaaggttgagg

SEQ ID NO: 56 - DNA, 24 bp, Artificial Sequence
  Description of Artificial Sequence Synthetic primer
agtctaaaccgtaaagaaacatgg

SEQ ID NO: 57 - DNA, 26 bp, Artificial Sequence
  Description of Artificial Sequence Synthetic primer
aaaggttgaggaattaaaggaagtgg

SEQ ID NO: 58 - DNA, 26 bp, Artificial Sequence
  Description of Artificial Sequence Synthetic primer
tcaaaatgataagacaatcccaaacg

SEQ ID NO: 59 - DNA, 25 bp, Artificial Sequence
  Description of Artificial Sequence Synthetic primer
tgaaaaattcaaagacgaggatggg

SEQ ID NO: 60 - DNA, 20 bp, Artificial Sequence
  Description of Artificial Sequence Synthetic primer
aagggcctctctagggctcg

SEQ ID NO: 61 - DNA, 21 bp, Artificial Sequence
  Description of Artificial Sequence Synthetic primer
acacttaaccgagtttttggc

SEQ ID NO: 62 - DNA, 20 bp, Artificial Sequence
  Description of Artificial Sequence Synthetic primer
tgctgacttcacaaagctcc

SEQ ID NO: 63 - DNA, 23 bp, Artificial Sequence
  Description of Artificial Sequence Synthetic primer
tgcaagagataggattgtggaat

SEQ ID NO: 64 - DNA, 26 bp, Artificial Sequence
  Description of Artificial Sequence Synthetic primer
gcatgagttaggatctcaagttcttc

SEQ ID NO: 65 - DNA, 27 bp, Artificial Sequence
  Description of Artificial Sequence Synthetic primer
tgggatataaattgtgtggataaactg

SEQ ID NO: 66 - DNA, 26 bp, Artificial Sequence
  Description of Artificial Sequence Synthetic primer
gcacttgctcaaactcttcataacaa

SEQ ID NO: 67 - DNA, 23 bp, Artificial Sequence
  Description of Artificial Sequence Synthetic primer
tgtttgcatagaggactcatccc

SEQ ID NO: 68 - DNA, 23 bp, Artificial Sequence
  Description of Artificial Sequence Synthetic primer
agttttcattccaaccaaagagc

SEQ ID NO: 69 - DNA, 23 bp, Artificial Sequence
  Description of Artificial Sequence Synthetic primer
tttggatgaagctcgatgtttgc

SEQ ID NO: 70 - DNA, 27 bp, Artificial Sequence
  Description of Artificial Sequence Synthetic primer
agtaacagatgctctaataatctttcg

SEQ ID NO: 71 - DNA, 21 bp, Artificial Sequence
  Description of Artificial Sequence Synthetic primer
atgaggaacattcagcagtgg

SEQ ID NO: 72 - DNA, 24 bp, Artificial Sequence
  Description of Artificial Sequence Synthetic primer
agcacgaagtaatataggtgaagc

SEQ ID NO: 73 - DNA, 22 bp, Artificial Sequence
  Description of Artificial Sequence Synthetic primer
atggcgtttgtgaagaggaagc

SEQ ID NO: 74 - DNA, 24 bp, Artificial Sequence
  Description of Artificial Sequence Synthetic primer
aattggatgaataagcaaagcagc

SEQ ID NO: 75 - DNA, 28 bp, Artificial Sequence
  Description of Artificial Sequence Synthetic primer
tccttagttctacaaaattagcaagagc

SEQ ID NO: 76 - DNA, 24 bp, Artificial Sequence
  Description of Artificial Sequence Synthetic primer
gtaatcaaaagaccaaatgggagg

SEQ ID NO: 77 - DNA, 26 bp, Artificial Sequence
  Description of Artificial Sequence Synthetic primer
acaagtagattgaatgagctagaagc

SEQ ID NO: 78 - DNA, 24 bp, Artificial Sequence
  Description of Artificial Sequence Synthetic primer
tcataaccatgttgtcgtagaagc

SEQ ID NO: 79 - DNA, 22 bp, Artificial Sequence
  Description of Artificial Sequence Synthetic primer
gttcaaggcaagaaggagtagc

SEQ ID NO: 80 - DNA, 21 bp, Artificial Sequence
  Description of Artificial Sequence Synthetic primer
tttgctccatcgtcatcgtcg

SEQ ID NO: 81 - DNA, 20 bp, Artificial Sequence
  Description of Artificial Sequence Synthetic primer
attatgcagcgacgatgacg

SEQ ID NO: 82 - DNA, 26 bp, Artificial Sequence
  Description of Artificial Sequence Synthetic primer
ccttgcttctgatcttgttattctcc

SEQ ID NO: 83 - DNA, 24 bp, Artificial Sequence
  Description of Artificial Sequence Synthetic primer
tggagtgtttcttatggactgtgg

SEQ ID NO: 84 - DNA, 24 bp, Artificial Sequence
  Description of Artificial Sequence Synthetic primer
ctctcaacagcattagtgaaaagc

SEQ ID NO: 85 - DNA, 24 bp, Artificial Sequence
  Description of Artificial Sequence Synthetic primer
tgtgaaagcaatggatgatttacc

SEQ ID NO: 86 - DNA, 23 bp, Artificial Sequence
  Description of Artificial Sequence Synthetic primer
aattttggtctcctaacacatcg

SEQ ID NO: 87 - DNA, 21 bp, Artificial Sequence
  Description of Artificial Sequence Synthetic primer
ggcgaaatggtttcacagtgg

SEQ ID NO: 88 - DNA, 21 bp, Artificial Sequence
  Description of Artificial Sequence Synthetic primer
cgtagaatcatggatgcgtgg

SEQ ID NO: 89 - DNA, 30 bp, Artificial Sequence
  Description of Artificial Sequence Synthetic primer
aaataatgaagatgaaaatatggactctcc

SEQ ID NO: 90 - DNA, 26 bp, Artificial Sequence
  Description of Artificial Sequence Synthetic primer
gataaactatcctgagaagcatgtcc

SEQ ID NO: 91 - DNA, 23 bp, Artificial Sequence
  Description of Artificial Sequence Synthetic primer
aaactatcaaccttcactttggc

SEQ ID NO: 92 - DNA, 22 bp, Artificial Sequence
  Description of Artificial Sequence Synthetic primer
ttcctcttttgctctcttcacc

SEQ ID NO: 93 - DNA, 26 bp, Artificial Sequence
  Description of Artificial Sequence Synthetic primer
tgcaaagacttggaatctcttatcac

SEQ ID NO: 94 - DNA, 22 bp, Artificial Sequence
  Description of Artificial Sequence Synthetic primer
gagacaccggataaccatgttg

SEQ ID NO: 95 - DNA, 23 bp, Artificial Sequence
  Description of Artificial Sequence Synthetic primer
gaggcaattttatggtgtgtacc

SEQ ID NO: 96 - DNA, 26 bp, Artificial Sequence
  Description of Artificial Sequence Synthetic primer
tctttgttgtttctcttgtctcttcc

SEQ ID NO: 97 - DNA, 20 bp, Artificial Sequence
  Description of Artificial Sequence Synthetic primer
tgggaaagcactggtatggg

SEQ ID NO: 98 - DNA, 23 bp, Artificial Sequence
  Description of Artificial Sequence Synthetic primer
agctccaattcatctagtgtacc

SEQ ID NO: 99 - DNA, 21 bp, Artificial Sequence
  Description of Artificial Sequence Synthetic primer
gtgggaaagcactggtatggg

SEQ ID NO: 100 - DNA, 25 bp, Artificial Sequence
  Description of Artificial Sequence Synthetic primer
aatttttgtaatgtggctcaaatgc

SEQ ID NO: 101 - DNA, 24 bp, Artificial Sequence
  Description of Artificial Sequence Synthetic primer
tgggatattagtgctatggatggg

SEQ ID NO: 102 - DNA, 28 bp, Artificial Sequence
  Description of Artificial Sequence Synthetic primer
ttctgaaggaattttattatatggaggc

SEQ ID NO: 103 - DNA, 25 bp, Artificial Sequence
  Description of Artificial Sequence Synthetic primer
tagtgaatccaaacaaggaagatgc

SEQ ID NO: 104 - DNA, 25 bp, Artificial Sequence
  Description of Artificial Sequence Synthetic primer
tcatctgtaagacgtaaaatcatcg

SEQ ID NO: 105 - DNA, 23 bp, Artificial Sequence
  Description of Artificial Sequence Synthetic primer
gatggcgtgtttgaagaagaagc

SEQ ID NO: 106 - DNA, 26 bp, Artificial Sequence
  Description of Artificial Sequence Synthetic primer
tgatagtacatgatcttttgtttggc

SEQ ID NO: 107 - DNA, 26 bp, Artificial Sequence
  Description of Artificial Sequence Synthetic primer
ttctcgaagatcagcaaactatcaac

SEQ ID NO: 108 - DNA, 25 bp, Artificial Sequence
  Description of Artificial Sequence Synthetic primer
ttgaaaggggtagaaagtgattgta

SEQ ID NO: 109 - DNA, 25 bp, Artificial Sequence
  Description of Artificial Sequence Synthetic primer
aggtaagagtaatggtgaagagagc

SEQ ID NO: 110 - DNA, 25 bp, Artificial Sequence
  Description of Artificial Sequence Synthetic primer
cattctcaaagtggtaagagattcc

SEQ ID NO: 111 - DNA, 22 bp, Artificial Sequence
  Description of Artificial Sequence Synthetic primer
gagcaaaagaggaggagaagcc

SEQ ID NO: 112 - DNA, 26 bp, Artificial Sequence
  Description of Artificial Sequence Synthetic primer
ggcatagacatttttgttggtgttgc

SEQ ID NO: 113 - DNA, 23 bp, Artificial Sequence
  Description of Artificial Sequence Synthetic primer
gaggcaattttatggtgtcttcc

SEQ ID NO: 114 - DNA, 22 bp, Artificial Sequence
  Description of Artificial Sequence Synthetic primer
tgttgtgtgtcttgtctcttcc

SEQ ID NO: 115 - DNA, 20 bp, Artificial Sequence
  Description of Artificial Sequence Synthetic primer
tgggaaagcactgatatggg

SEQ ID NO: 116 - DNA, 23 bp, Artificial Sequence
  Description of Artificial Sequence Synthetic primer
agctccaattcatctagtgtacc

SEQ ID NO: 117 - DNA, 24 bp, Artificial Sequence
  Description of Artificial Sequence Synthetic primer
tgggatattagtgctatggatggg

SEQ ID NO: 118 - DNA, 28 bp, Artificial Sequence
  Description of Artificial Sequence Synthetic primer
ttctgaaggaattttattatatggaggc

SEQ ID NO: 119 - DNA, 26 bp, Artificial Sequence
  Description of Artificial Sequence Synthetic primer
agctttgaagagtacattgagaatgc

SEQ ID NO: 120 - DNA, 26 bp, Artificial Sequence
  Description of Artificial Sequence Synthetic primer
atcatctgtaagtcgtaaaatcatcg

SEQ ID NO: 121 - DNA, 22 bp, Artificial Sequence
  Description of Artificial Sequence Synthetic primer
ggcgatgttcccaaatcaatcc

SEQ ID NO: 122 - DNA, 23 bp, Artificial Sequence
  Description of Artificial Sequence Synthetic primer
aacattggagagtagtcatcacc

SEQ ID NO: 123 - DNA, 26 bp, Artificial Sequence
  Description of Artificial Sequence Synthetic primer
gatgatgatggtgatgactactctcc

SEQ ID NO: 124 - DNA, 26 bp, Artificial Sequence
  Description of Artificial Sequence Synthetic primer
gatagtacatgatcttttgtatggcg

SEQ ID NO: 125 - DNA, 22 bp, Artificial Sequence
  Description of Artificial Sequence Synthetic primer
gatcagcaaactatcaaccccc

SEQ ID NO: 126 - DNA, 23 bp, Artificial Sequence
  Description of Artificial Sequence Synthetic primer
tcaccatcactcttacttcttcc

SEQ ID NO: 127 - DNA, 26 bp, Artificial Sequence
  Description of Artificial Sequence Synthetic primer
acttggaatctcttatcactttgagg

SEQ ID NO: 128 - DNA, 28 bp, Artificial Sequence
  Description of Artificial Sequence Synthetic primer
agagaattggcatacacattattattgg

SEQ ID NO: 129 - DNA, 23 bp, Artificial Sequence
  Description of Artificial Sequence Synthetic primer
gaggcaattttatggtgtgtacc

SEQ ID NO: 130 - DNA, 25 bp, Artificial Sequence
  Description of Artificial Sequence Synthetic primer
tttctcttgtctcttccaaaatacc

SEQ ID NO: 131 - DNA, 22 bp, Artificial Sequence
  Description of Artificial Sequence Synthetic primer
tggtgggaaagcactgatatgg

SEQ ID NO: 132 - DNA, 22 bp, Artificial Sequence
  Description of Artificial Sequence Synthetic primer
aatgtggctcaaatgcaactcc

SEQ ID NO: 133 - DNA, 20 bp, Artificial Sequence
  Description of Artificial Sequence Synthetic primer
tgggaaagcactgatatggg

SEQ ID NO: 134 - DNA, 23 bp, Artificial Sequence
  Description of Artificial Sequence Synthetic primer
agctccaattcatctagtgtacc

SEQ ID NO: 135 - DNA, 24 bp, Artificial Sequence
  Description of Artificial Sequence Synthetic primer
tgggatattagtgctatggatggg

SEQ ID NO: 136 - DNA, 28 bp, Artificial Sequence
  Description of Artificial Sequence Synthetic primer
ttctgaaggaattttattatatggaggc

SEQ ID NO: 137 - DNA, 25 bp, Artificial Sequence
  Description of Artificial Sequence Synthetic primer
gctttgaagagtacattgagaatgc

SEQ ID NO: 138 - DNA, 25 bp, Artificial Sequence
  Description of Artificial Sequence Synthetic primer
cgatgaatgtcgtattatggtaggg

SEQ ID NO: 139 - DNA, 24 bp, Artificial Sequence
  Description of Artificial Sequence Synthetic primer
gtgaatccaaacaaggaaaatgcc

SEQ ID NO: 140 - DNA, 27 bp, Artificial Sequence
  Description of Artificial Sequence Synthetic primer
tgaagttcccatatcatctttaagtcg

SEQ ID NO: 141 - DNA, 24 bp, Artificial Sequence
  Description of Artificial Sequence Synthetic primer
tgaattgaaaagaggcgatgttcc

SEQ ID NO: 142 - DNA, 22 bp, Artificial Sequence
  Description of Artificial Sequence Synthetic primer
acattggagagtagtcatcacc

SEQ ID NO: 143 - DNA, 24 bp, Artificial Sequence
  Description of Artificial Sequence Synthetic primer
tggtatatctgaagaggaagctcg

SEQ ID NO: 144 - DNA, 25 bp, Artificial Sequence
  Description of Artificial Sequence Synthetic primer
agaacattggagagtagtcatcacc

SEQ ID NO: 145 - DNA, 22 bp, Artificial Sequence
  Description of Artificial Sequence Synthetic primer
gcatagcttttcaccaatttgc

SEQ ID NO: 146 - DNA, 21 bp, Artificial Sequence
  Description of Artificial Sequence Synthetic primer
gggagggatgatgatgaagca

SEQ ID NO: 147 - DNA, 23 bp, Artificial Sequence
  Description of Artificial Sequence Synthetic primer
caatgtactgtggtcgataaccc

SEQ ID NO: 148 - DNA, 25 bp, Artificial Sequence
  Description of Artificial Sequence Synthetic primer
gaacaaaatcaaaagaccaaatggg

SEQ ID NO: 149 - DNA, 24 bp, Artificial Sequence
  Description of Artificial Sequence Synthetic primer
tggagaaagatgtgaaaaggatgc

SEQ ID NO: 150 - DNA, 23 bp, Artificial Sequence
  Description of Artificial Sequence Synthetic primer
attggcgtagaagcctaaattgg

SEQ ID NO: 151 - DNA, 23 bp, Artificial Sequence
  Description of Artificial Sequence Synthetic primer
tcacaagacaggagagttcaagg

SEQ ID NO: 152 - DNA, 20 bp, Artificial Sequence
  Description of Artificial Sequence Synthetic primer
tgaagtggcatctccaaagc

SEQ ID NO: 153 - DNA, 23 bp, Artificial Sequence
  Description of Artificial Sequence Synthetic primer
tggtggaaggattctaaacttgg

SEQ ID NO: 154 - DNA, 25 bp, Artificial Sequence
  Description of Artificial Sequence Synthetic primer
ctccaatgttccataaatgtcatgc

SEQ ID NO: 155 - DNA, 23 bp, Artificial Sequence
  Description of Artificial Sequence Synthetic primer
gcaagttggagtaagatttgagc

SEQ ID NO: 156 - DNA, 25 bp, Artificial Sequence
  Description of Artificial Sequence Synthetic primer
attagtgaaaagttgtagttcctcc

SEQ ID NO: 157 - DNA, 25 bp, Artificial Sequence
  Description of Artificial Sequence Synthetic primer
aagaggcaaaatggttttatagtgg

SEQ ID NO: 158 - DNA, 24 bp, Artificial Sequence
  Description of Artificial Sequence Synthetic primer
ttggtaacaggatttgtaaaagcg

SEQ ID NO: 159 - DNA, 20 bp, Artificial Sequence
  Description of Artificial Sequence Synthetic primer
ggatggttgtctgtgggagg

SEQ ID NO: 160 - DNA, 21 bp, Artificial Sequence
  Description of Artificial Sequence Synthetic primer
tgcatggcgaactatgttagg

SEQ ID NO: 161 - DNA, 21 bp, Artificial Sequence
  Description of Artificial Sequence Synthetic primer
tgcaaaaatcttggtagagcg

SEQ ID NO: 162 - DNA, 25 bp, Artificial Sequence
  Description of Artificial Sequence Synthetic primer
tggggataggagtaataatcaaccc

SEQ ID NO: 163 - DNA, 29 bp, Artificial Sequence
  Description of Artificial Sequence Synthetic primer
atattcaagtcttagcttcatctcaatta

SEQ ID NO: 164 - DNA, 21 bp, Artificial Sequence
  Description of Artificial Sequence Synthetic primer
atcgccccaaatagaagggtg

SEQ ID NO: 165 - DNA, 28 bp, Artificial Sequence
  Description of Artificial Sequence Synthetic primer
tttgagagtgaaattgagaaattgttgg

SEQ ID NO: 166 - DNA, 30 bp, Artificial Sequence
  Description of Artificial Sequence Synthetic primer
taaatccatgttgtcttaataatctaaacc

SEQ ID NO: 167 - DNA, 23 bp, Artificial Sequence
  Description of Artificial Sequence Synthetic primer
ggtttgcttagcttgtatgaggc

SEQ ID NO: 168 - DNA, 25 bp, Artificial Sequence
  Description of Artificial Sequence Synthetic primer
gcctctctatggtctttcttagagg

SEQ ID NO: 169 - DNA, 23 bp, Artificial Sequence
  Description of Artificial Sequence Synthetic primer
tggtggaaagagcatgagtttgc

SEQ ID NO: 170 - DNA, 25 bp, Artificial Sequence
  Description of Artificial Sequence Synthetic primer
attgaggctaatgcaatgactttgg

SEQ ID NO: 171 - DNA, 21 bp, Artificial Sequence
  Description of Artificial Sequence Synthetic primer
agctcgatggttgagtgaagg

SEQ ID NO: 172 - DNA, 20 bp, Artificial Sequence
  Description of Artificial Sequence Synthetic primer
ctagcaaggagagtggaagc

SEQ ID NO: 173 - DNA, 24 bp, Artificial Sequence
  Description of Artificial Sequence Synthetic primer
tgagcaaaagagaaatcacatacc

SEQ ID NO: 174 - DNA, 20 bp, Artificial Sequence
  Description of Artificial Sequence Synthetic primer
tttctttccagtgggtgtcc

SEQ ID NO: 175 - DNA, 24 bp, Artificial Sequence
  Description of Artificial Sequence Synthetic primer
tgaaacaatatggggtatcagagg

SEQ ID NO: 176 - DNA, 24 bp, Artificial Sequence
  Description of Artificial Sequence Synthetic primer
aatgctttctttgagcacttttcc

SEQ ID NO: 177 - DNA, 20 bp, Artificial Sequence
  Description of Artificial Sequence Synthetic primer
gcattgtatggctgttcacc

SEQ ID NO: 178 - DNA, 20 bp, Artificial Sequence
  Description of Artificial Sequence Synthetic primer
tcgaaagaccaaatcggagg

SEQ ID NO: 179 - DNA, 25 bp, Artificial Sequence
  Description of Artificial Sequence Synthetic primer
tcacaccaaaaacatctattagtcc

SEQ ID NO: 180 - DNA, 23 bp, Artificial Sequence
  Description of Artificial Sequence Synthetic primer
caaatcggaggttcataattggc

SEQ ID NO: 181 - DNA, 25 bp, Artificial Sequence
  Description of Artificial Sequence Synthetic primer
aagaaagaagtgacaagaatgctcc

SEQ ID NO: 182 - DNA, 20 bp, Artificial Sequence
  Description of Artificial Sequence Synthetic primer
actgtatagccatgttggcg

SEQ ID NO: 183 - DNA, 25 bp, Artificial Sequence
  Description of Artificial Sequence Synthetic primer
gaaattaactctttagccctactcg

SEQ ID NO: 184 - DNA, 22 bp, Artificial Sequence
  Description of Artificial Sequence Synthetic primer
agcctaaattcgagagcaatgg

SEQ ID NO: 185 - DNA, 22 bp, Artificial Sequence
  Description of Artificial Sequence Synthetic primer
tgctttcaaggataagagaggg

SEQ ID NO: 186 - DNA, 24 bp, Artificial Sequence
  Description of Artificial Sequence Synthetic primer
tcttcctcattctccattttctcc

SEQ ID NO: 187 - DNA, 23 bp, Artificial Sequence
  Description of Artificial Sequence Synthetic primer
catggagaaaatggagaatgagg

SEQ ID NO: 188 - DNA, 21 bp, Artificial Sequence
  Description of Artificial Sequence Synthetic primer
tcgcaaactcgaacaaagtcg

SEQ ID NO: 189 - DNA, 25 bp, Artificial Sequence
  Description of Artificial Sequence Synthetic primer
ttgctagagatagattgatggaagc

SEQ ID NO: 190 - DNA, 25 bp, Artificial Sequence
  Description of Artificial Sequence Synthetic primer
aaagctctaattcctccaaagttcc

SEQ ID NO: 191 - DNA, 23 bp, Artificial Sequence
  Description of Artificial Sequence Synthetic primer
accagattacatgaagatgcctt

SEQ ID NO: 192 - DNA, 23 bp, Artificial Sequence
  Description of Artificial Sequence Synthetic primer
ctaatacatcgaaccccatctca

SEQ ID NO: 193 - DNA, 25 bp, Artificial Sequence
  Description of Artificial Sequence Synthetic primer
ggtattatagtggataccaaccaac

SEQ ID NO: 194 - DNA, 20 bp, Artificial Sequence
  Description of Artificial Sequence Synthetic primer
aagccaacccaactcagtgt

SEQ ID NO: 195 - DNA, 21 bp, Artificial Sequence
  Description of Artificial Sequence Synthetic primer
acactgagttgggttggcttt

SEQ ID NO: 196 - DNA, 23 bp, Artificial Sequence
  Description of Artificial Sequence Synthetic primer
gttcctaaatcatctgcaagcct

SEQ ID NO: 197 - DNA, 20 bp, Artificial Sequence
  Description of Artificial Sequence Synthetic primer
ttgaatagaggcgacgttcc

SEQ ID NO: 198 - DNA, 23 bp, Artificial Sequence
  Description of Artificial Sequence Synthetic primer
tgctgttctagccatatttttgc

SEQ ID NO: 199 - DNA, 25 bp, Artificial Sequence
  Description of Artificial Sequence Synthetic primer
tctagtcaagtgttagcttcatctc

SEQ ID NO: 200 - DNA, 22 bp, Artificial Sequence
  Description of Artificial Sequence Synthetic primer
tttgttgttggtcgaatgatgt

SEQ ID NO: 201 - DNA, 25 bp, Artificial Sequence
  Description of Artificial Sequence Synthetic primer
agggacaagttgaagaattgaaaga

SEQ ID NO: 202 - DNA, 23 bp, Artificial Sequence
  Description of Artificial Sequence Synthetic primer
catggttggagtagtagtagtgt

SEQ ID NO: 203 - DNA, 24 bp, Artificial Sequence
  Description of Artificial Sequence Synthetic primer
gagagtggtaagtttaaggaaagc

SEQ ID NO: 204 - DNA, 22 bp, Artificial Sequence
  Description of Artificial Sequence Synthetic primer
cctagcataaagcctcactagg

SEQ ID NO: 205 - DNA, 22 bp, Artificial Sequence
  Description of Artificial Sequence Synthetic primer
ggctcttgctttcactaccacc

SEQ ID NO: 206 - DNA, 24 bp, Artificial Sequence
  Description of Artificial Sequence Synthetic primer
cctcactagggtttttctcaaagg

SEQ ID NO: 207 - DNA, 22 bp, Artificial Sequence
  Description of Artificial Sequence Synthetic primer
ggtggaaagaattagacttggc

SEQ ID NO: 208 - DNA, 20 bp, Artificial Sequence
  Description of Artificial Sequence Synthetic primer
atgtcccacctgagaattgc

SEQ ID NO: 209 - DNA, 29 bp, Artificial Sequence
  Description of Artificial Sequence Synthetic primer
acttagtccagattatttgaagacatatt

SEQ ID NO: 210 - DNA, 25 bp, Artificial Sequence
  Description of Artificial Sequence Synthetic primer
tgcatagtgaagtttgtatctctct

SEQ ID NO: 211 - DNA, 24 bp, Artificial Sequence
  Description of Artificial Sequence Synthetic primer
ttgagacctcttttgttggaatgc

SEQ ID NO: 212 - DNA, 24 bp, Artificial Sequence
  Description of Artificial Sequence Synthetic primer
aaaatctttggttgtgtagagagc

SEQ ID NO: 213 - DNA, 21 bp, Artificial Sequence
  Description of Artificial Sequence Synthetic primer
atggtgtatcggaacaagagg

SEQ ID NO: 214 - DNA, 18 bp, Artificial Sequence
  Description of Artificial Sequence Synthetic primer
aacgcagcaacactgtcc

SEQ ID NO: 215 - DNA, 28 bp, Artificial Sequence
  Description of Artificial Sequence Synthetic primer
aagtgcttgacattatctacaaagaagg

SEQ ID NO: 216 - DNA, 27 bp, Artificial Sequence
  Description of Artificial Sequence Synthetic primer
taatgggatgggatctataagcaacgc

SEQ ID NO: 217 - DNA, 23 bp, Artificial Sequence
  Description of Artificial Sequence Synthetic primer
atggcttttcaccaatttgctcc

SEQ ID NO: 218 - DNA, 23 bp, Artificial Sequence
  Description of Artificial Sequence Synthetic primer
tcgtagaactagggttatcgacc

SEQ ID NO: 219 - DNA, 23 bp, Artificial Sequence
  Description of Artificial Sequence Synthetic primer
tttgctccatcatcatccctccc

SEQ ID NO: 220 - DNA, 25 bp, Artificial Sequence
  Description of Artificial Sequence Synthetic primer
gaggtccatagttggctgatcttcg

SEQ ID NO: 221 - DNA, 25 bp, Artificial Sequence
  Description of Artificial Sequence Synthetic primer
ttgaagaaagaagtgacaagaatgg

SEQ ID NO: 222 - DNA, 23 bp, Artificial Sequence
  Description of Artificial Sequence Synthetic primer
ggtaagatattccaagcctttgc

SEQ ID NO: 223 - DNA, 25 bp, Artificial Sequence
  Description of Artificial Sequence Synthetic primer
tacatggagaaaatggagaatgagg

SEQ ID NO: 224 - DNA, 20 bp, Artificial Sequence
  Description of Artificial Sequence Synthetic primer
tgaagcggaagctcgaaagc

SEQ ID NO: 225 - DNA, 20 bp, Artificial Sequence
  Description of Artificial Sequence Synthetic primer
atcacgctttcgagcttccg

SEQ ID NO: 226 - DNA, 24 bp, Artificial Sequence
  Description of Artificial Sequence Synthetic primer
aaatcttcttggtgtgttgattgc

SEQ ID NO: 227 - DNA, 25 bp, Artificial Sequence
  Description of Artificial Sequence Synthetic primer
ttgctagagatagattgatggaagc

SEQ ID NO: 228 - DNA, 25 bp, Artificial Sequence
  Description of Artificial Sequence Synthetic primer
aaagctctaattcctccaaagttcc

SEQ ID NO: 229 - DNA, 24 bp, Artificial Sequence
  Description of Artificial Sequence Synthetic primer
gttaccagattacatgaagatgcc

SEQ ID NO: 230 - DNA, 23 bp, Artificial Sequence
  Description of Artificial Sequence Synthetic primer
actaatacatcgaaccccatctc

SEQ ID NO: 231 - DNA, 22 bp, Artificial Sequence
  Description of Artificial Sequence Synthetic primer
agttgggttggctttcaatagg

SEQ ID NO: 232 - DNA, 23 bp, Artificial Sequence
  Description of Artificial Sequence Synthetic primer
atgttcctaaatcatctgcaagc

SEQ ID NO: 233 - DNA, 22 bp, Artificial Sequence
  Description of Artificial Sequence Synthetic primer
tagaggcgacgttcctaaatcg

SEQ ID NO: 234 - DNA, 23 bp, Artificial Sequence
  Description of Artificial Sequence Synthetic primer
tgctgttctagccatatttttgc

SEQ ID NO: 235 - DNA, 25 bp, Artificial Sequence
  Description of Artificial Sequence Synthetic primer
tctagtcaagtgttagcttcatctc

SEQ ID NO: 236 - DNA, 24 bp, Artificial Sequence
  Description of Artificial Sequence Synthetic primer
tgttgttggtcgaatgatgttttg

SEQ ID NO: 237 - DNA, 27 bp, Artificial Sequence
  Description of Artificial Sequence Synthetic primer
aagaagttgttaggaaagagatattcc

SEQ ID NO: 238 - DNA, 27 bp, Artificial Sequence
  Description of Artificial Sequence Synthetic primer
agatgaaatgttaaatccatgttgtcg

SEQ ID NO: 239 - DNA, 25 bp, Artificial Sequence
  Description of Artificial Sequence Synthetic primer
tgagagtggtaagtttaaggaaagc

SEQ ID NO: 240 - DNA, 21 bp, Artificial Sequence
  Description of Artificial Sequence Synthetic primer
ggtggtagtgaaagcaagagc

SEQ ID NO: 241 - DNA, 22 bp, Artificial Sequence
  Description of Artificial Sequence Synthetic primer
ggctcttgctttcactaccacc

SEQ ID NO: 242 - DNA, 24 bp, Artificial Sequence
  Description of Artificial Sequence Synthetic primer
cctcactagggtttttctcaaagg

SEQ ID NO: 243 - DNA, 21 bp, Artificial Sequence
  Description of Artificial Sequence Synthetic primer
tgagaaaaaccctagtgaggc

SEQ ID NO: 244 - DNA, 27 bp, Artificial Sequence
  Description of Artificial Sequence Synthetic primer
ttgtagtagattgaagtccaactttgc

SEQ ID NO: 245 - DNA, 23 bp, Artificial Sequence
  Description of Artificial Sequence Synthetic primer
ttagacttggcaaacaaactacc

SEQ ID NO: 246 - DNA, 21 bp, Artificial Sequence
  Description of Artificial Sequence Synthetic primer
tatgtcccacctgagaattgc

SEQ ID NO: 247 - DNA, 30 bp, Artificial Sequence
  Description of Artificial Sequence Synthetic primer
acttagtccagattatttgaagacatatta

SEQ ID NO: 248 - DNA, 26 bp, Artificial Sequence
  Description of Artificial Sequence Synthetic primer
ttgcatagtgaagtttgtatctctct

SEQ ID NO: 249 - DNA, 22 bp, Artificial Sequence
  Description of Artificial Sequence Synthetic primer
ttccatgaagcacaatggttga

SEQ ID NO: 250 - DNA, 23 bp, Artificial Sequence
  Description of Artificial Sequence Synthetic primer
gcattccaacaaaagaggtctca

SEQ ID NO: 251 - DNA, 24 bp, Artificial Sequence
  Description of Artificial Sequence Synthetic primer
ggttacccaatgttgattgagacc

SEQ ID NO: 252 - DNA, 25 bp, Artificial Sequence
  Description of Artificial Sequence Synthetic primer
ggttgtgtagagagccattcaaata

SEQ ID NO: 253 - DNA, 21 bp, Artificial Sequence
  Description of Artificial Sequence Synthetic primer
tggtgtatcggaacaagaggc

SEQ ID NO: 254 - DNA, 19 bp, Artificial Sequence
  Description of Artificial Sequence Synthetic primer
caacgcagcaacactgtcc

SEQ ID NO: 255 - DNA, 20 bp, Artificial Sequence
  Description of Artificial Sequence Synthetic primer
acgcatcttttcgtcccttt

SEQ ID NO: 256 - DNA, 23 bp, Artificial Sequence
  Description of Artificial Sequence Synthetic primer
tgagatcaccgttactcctgata

SEQ ID NO: 257 - DNA, 21 bp, Artificial Sequence
  Description of Artificial Sequence Synthetic primer
agcagacccatttgatgaagg

SEQ ID NO: 258 - DNA, 20 bp, Artificial Sequence
  Description of Artificial Sequence Synthetic primer
cagcaggaacgaagagaccg

SEQ ID NO: 259 - DNA, 21 bp, Artificial Sequence
  Description of Artificial Sequence Synthetic primer
tcgacacaaggctaagagagg

SEQ ID NO: 260 - DNA, 20 bp, Artificial Sequence
  Description of Artificial Sequence Synthetic primer
gtccttggctgtgaacttgg

SEQ ID NO: 261 - DNA, 20 bp, Artificial Sequence
  Description of Artificial Sequence Synthetic primer
caagttcacagccaaggacc

SEQ ID NO: 262 - DNA, 25 bp, Artificial Sequence
  Description of Artificial Sequence Synthetic primer
ttatcaatttccattctatgcaggg

SEQ ID NO: 263 - DNA, 22 bp, Artificial Sequence
  Description of Artificial Sequence Synthetic primer
ggtggcgagacattggtttagc

SEQ ID NO: 264 - DNA, 22 bp, Artificial Sequence
  Description of Artificial Sequence Synthetic primer
atggattttgtaagcgcaaccc

SEQ ID NO: 265 - DNA, 25 bp, Artificial Sequence
  Description of Artificial Sequence Synthetic primer
tgctatacaaaaacttccagactcc

SEQ ID NO: 266 - DNA, 20 bp, Artificial Sequence
  Description of Artificial Sequence Synthetic primer
tgtaaagggctccatccacg

SEQ ID NO: 267 - DNA, 19 bp, Artificial Sequence
  Description of Artificial Sequence Synthetic primer
gggcaagtttgtgcgaagc

SEQ ID NO: 268 - DNA, 21 bp, Artificial Sequence
  Description of Artificial Sequence Synthetic primer
tggcactaccaaagtcatccc

SEQ ID NO: 269 - DNA, 19 bp, Artificial Sequence
  Description of Artificial Sequence Synthetic primer
atggtgtggtcagctcagg

SEQ ID NO: 270 - DNA, 21 bp, Artificial Sequence
  Description of Artificial Sequence Synthetic primer
tggcactaccaaagtcatccc

SEQ ID NO: 271 - DNA, 22 bp, Artificial Sequence
  Description of Artificial Sequence Synthetic primer
agaatcaagaaggacatgacgg

SEQ ID NO: 272 - DNA, 21 bp, Artificial Sequence
  Description of Artificial Sequence Synthetic primer
tttgttgaggcacttccatgc

SEQ ID NO: 273 - DNA, 21 bp, Artificial Sequence
  Description of Artificial Sequence Synthetic primer
aagtgcctcaacaaagaatgc

SEQ ID NO: 274 - DNA, 20 bp, Artificial Sequence
  Description of Artificial Sequence Synthetic primer
gttcttccaagtggggtagg

SEQ ID NO: 275 - DNA, 28 bp, Artificial Sequence
  Description of Artificial Sequence Synthetic primer
atggcgttgtcaataatgtcttcttacg

SEQ ID NO: 276 - DNA, 24 bp, Artificial Sequence
  Description of Artificial Sequence Synthetic primer
gagaactcgaaagtgatgaggagg

SEQ ID NO: 277 - DNA, 21 bp, Artificial Sequence
  Description of Artificial Sequence Synthetic primer
agcagacccatttgatgaagg

SEQ ID NO: 278 - DNA, 19 bp, Artificial Sequence
  Description of Artificial Sequence Synthetic primer
agcaggaacgaagtgaccg

SEQ ID NO: 279 - DNA, 29 bp, Artificial Sequence
  Description of Artificial Sequence Synthetic primer
gataaatacagggacgttttaagaaaagc

SEQ ID NO: 280 - DNA, 26 bp, Artificial Sequence
  Description of Artificial Sequence Synthetic primer
cctcgaagatatagtcaattcctagc

SEQ ID NO: 281 - DNA, 22 bp, Artificial Sequence
  Description of Artificial Sequence Synthetic primer
atatgaagcctcccatctatgc

SEQ ID NO: 282 - DNA, 23 bp, Artificial Sequence
  Description of Artificial Sequence Synthetic primer
ccattctcactgggatagacacc

SEQ ID NO: 283 - DNA, 22 bp, Artificial Sequence
  Description of Artificial Sequence Synthetic primer
ggtggcgagacattggtttagc

SEQ ID NO: 284 - DNA, 22 bp, Artificial Sequence
  Description of Artificial Sequence Synthetic primer
atggattttgtaagcgcaaccc

SEQ ID NO: 285 - DNA, 23 bp, Artificial Sequence
  Description of Artificial Sequence Synthetic primer
agaaaaacttccagactccatga

SEQ ID NO: 286 - DNA, 20 bp, Artificial Sequence
  Description of Artificial Sequence Synthetic primer
gctccatccacgcttttgat

SEQ ID NO: 287 - DNA, 24 bp, Artificial Sequence
  Description of Artificial Sequence Synthetic primer
tcaatgagtctagccatacgatct

SEQ ID NO: 288 - DNA, 20 bp, Artificial Sequence
  Description of Artificial Sequence Synthetic primer
tgtaaagggctccatccacg

SEQ ID NO: 289 - DNA, 19 bp, Artificial Sequence
  Description of Artificial Sequence Synthetic primer
gggcaagtttgtgcgaagc

SEQ ID NO: 290 - DNA, 21 bp, Artificial Sequence
  Description of Artificial Sequence Synthetic primer
tggcactaccaaagtcatccc

SEQ ID NO: 291 - DNA, 22 bp, Artificial Sequence
  Description of Artificial Sequence Synthetic primer
ggacatgacggatcttatgtgg

SEQ ID NO: 292 - DNA, 20 bp, Artificial Sequence
  Description of Artificial Sequence Synthetic primer
ttgttgaggcacttccatgc

SEQ ID NO: 293 - DNA, 19 bp, Artificial Sequence
  Description of Artificial Sequence Synthetic primer
atccagcgtttccaccacc

SEQ ID NO: 294 - DNA, 21 bp, Artificial Sequence
  Description of Artificial Sequence Synthetic primer
gttcttccaagtggggtaggc

SEQ ID NO: 295 - DNA, 24 bp, Artificial Sequence
  Description of Artificial Sequence Synthetic primer
accaatttgcttcatcatcatccc

SEQ ID NO: 296 - DNA, 22 bp, Artificial Sequence
  Description of Artificial Sequence Synthetic primer
aaatgggaggtccatagttggc

SEQ ID NO: 297 - DNA, 24 bp, Artificial Sequence
  Description of Artificial Sequence Synthetic primer
tgaaaaggatgctaattggagtgg

SEQ ID NO: 298 - DNA, 23 bp, Artificial Sequence
  Description of Artificial Sequence Synthetic primer
cgtagaagcctaaattggagagc

SEQ ID NO: 299 - DNA, 25 bp, Artificial Sequence
  Description of Artificial Sequence Synthetic primer
atcacaagacaggagagttcaaggc

SEQ ID NO: 300 - DNA, 24 bp, Artificial Sequence
  Description of Artificial Sequence Synthetic primer
attctccattgaagtggcatctcc

SEQ ID NO: 301 - DNA, 23 bp, Artificial Sequence
  Description of Artificial Sequence Synthetic primer
acttggagagaaattgcctttcg

SEQ ID NO: 302 - DNA, 24 bp, Artificial Sequence
  Description of Artificial Sequence Synthetic primer
tccaatgttccataaatgtcatgc

SEQ ID NO: 303 - DNA, 23 bp, Artificial Sequence
  Description of Artificial Sequence Synthetic primer
gcaagttggagtaagatttgagc

SEQ ID NO: 304 - DNA, 25 bp, Artificial Sequence
  Description of Artificial Sequence Synthetic primer
attagtgaaaagttgtagttcctcc

SEQ ID NO: 305 - DNA, 27 bp, Artificial Sequence
  Description of Artificial Sequence Synthetic primer
taagttaccagattatatgaagacagc

SEQ ID NO: 306 - DNA, 29 bp, Artificial Sequence
  Description of Artificial Sequence Synthetic primer
tttgtgaaattgtatgtaaagtagaaagc

SEQ ID NO: 307 - DNA, 22 bp, Artificial Sequence
  Description of Artificial Sequence Synthetic primer
agaatggatggttgtctgtggg

SEQ ID NO: 308 - DNA, 22 bp, Artificial Sequence
  Description of Artificial Sequence Synthetic primer
ggcgaactatgttaggatgacc

SEQ ID NO: 309 - DNA, 28 bp, Artificial Sequence
  Description of Artificial Sequence Synthetic primer
caattcaatgttatatgcacgatactgg

SEQ ID NO: 310 - DNA, 21 bp, Artificial Sequence
  Description of Artificial Sequence Synthetic primer
ctgagaagcatgtccatcgcc

SEQ ID NO: 311 - DNA, 27 bp, Artificial Sequence
  Description of Artificial Sequence Synthetic primer
attcaagtcttagcttcatctcaatta

SEQ ID NO: 312 - DNA, 22 bp, Artificial Sequence
  Description of Artificial Sequence Synthetic primer
cgatcaccccaaatagaagggt

SEQ ID NO: 313 - DNA, 28 bp, Artificial Sequence
  Description of Artificial Sequence Synthetic primer
tttgagagtgaaattgagaaattgttgg

SEQ ID NO: 314 - DNA, 30 bp, Artificial Sequence; identical to SEQ ID NO: 166
  Description of Artificial Sequence Synthetic primer

SEQ ID NO: 315 - DNA, 22 bp, Artificial Sequence
  Description of Artificial Sequence Synthetic primer
gcctaataaccgatgttccagg

SEQ ID NO: 316 - DNA, 25 bp, Artificial Sequence
  Description of Artificial Sequence Synthetic primer
gagatgtaaaacctagcatgaagcc

SEQ ID NO: 317 - DNA, 22 bp, Artificial Sequence
  Description of Artificial Sequence Synthetic primer
agaccatagagaggcttcatgc

SEQ ID NO: 318 - DNA, 28 bp, Artificial Sequence
  Description of Artificial Sequence Synthetic primer
cgtaatttcactaagttcctttttgtgg

SEQ ID NO: 319 - DNA, 22 bp, Artificial Sequence
  Description of Artificial Sequence Synthetic primer
ggtggaaggagcatgagtttgc

SEQ ID NO: 320 - DNA, 26 bp, Artificial Sequence
  Description of Artificial Sequence Synthetic primer
acctttgaattgctttggtaagaagc

SEQ ID NO: 321 - DNA, 27 bp, Artificial Sequence
  Description of Artificial Sequence Synthetic primer
gagtctcttatgtttcttctggtaacg

SEQ ID NO: 322 - DNA, 27 bp, Artificial Sequence
  Description of Artificial Sequence Synthetic primer
catgaacctagaaaggagagtagaagc

SEQ ID NO: 323 - DNA, 24 bp, Artificial Sequence
  Description of Artificial Sequence Synthetic primer
tgagcaaaagagaaatcacatacc

SEQ ID NO: 324 - DNA, 20 bp, Artificial Sequence
  Description of Artificial Sequence Synthetic primer
tttctttccagtgggtgtcc

SEQ ID NO: 325 - DNA, 20 bp, Artificial Sequence
  Description of Artificial Sequence Synthetic primer
ccagcagttgtgccctttcc

SEQ ID NO: 326 - DNA, 25 bp, Artificial Sequence
  Description of Artificial Sequence Synthetic primer
caatgctttctttgagcacttttcc

SEQ ID NO: 327 - DNA, 29 bp, Artificial Sequence
  Description of Artificial Sequence Synthetic primer
tcaagtcttagcttcatctcaattatgtg

SEQ ID NO: 328 - DNA, 21 bp, Artificial Sequence
  Description of Artificial Sequence Synthetic primer
gatcaccccaaatagaagggt

SEQ ID NO: 329 - DNA, 28 bp, Artificial Sequence
  Description of Artificial Sequence Synthetic primer
ttcgagagtgaaatcgagaaattattgg

SEQ ID NO: 330 - DNA, 28 bp, Artificial Sequence
  Description of Artificial Sequence Synthetic primer
atccactttgtcttaatagtctaaaccg

SEQ ID NO: 331 - DNA, 22 bp, Artificial Sequence
  Description of Artificial Sequence Synthetic primer
cttgctttcacaaccactcacc

SEQ ID NO: 332 - DNA, 25 bp, Artificial Sequence
  Description of Artificial Sequence Synthetic primer
cctctctatggtctttcttagaggc

SEQ ID NO: 333 - DNA, 22 bp, Artificial Sequence
  Description of Artificial Sequence Synthetic primer
ataaccgatgtttcaggtttgc

SEQ ID NO: 334 - DNA, 22 bp, Artificial Sequence
  Description of Artificial Sequence Synthetic primer
taaggtgagtggttgtgaaagc

SEQ ID NO: 335 - DNA, 28 bp, Artificial Sequence
  Description of Artificial Sequence Synthetic primer
gtgtatattatgaacccaaatactctcg

SEQ ID NO: 336 - DNA, 25 bp, Artificial Sequence
  Description of Artificial Sequence Synthetic primer
cctttgaattgctttggtaagaagc

SEQ ID NO: 337 - DNA, 28 bp, Artificial Sequence
  Description of Artificial Sequence Synthetic primer
gaactattggaggttattttgaagaagc

SEQ ID NO: 338 - DNA, 26 bp, Artificial Sequence
  Description of Artificial Sequence Synthetic primer
gagtggaagctgaaacaatcttaggg

SEQ ID NO: 339 - DNA, 24 bp, Artificial Sequence
  Description of Artificial Sequence Synthetic primer
tgagcaagagagaaatcacatacc

SEQ ID NO: 340 - DNA, 26 bp, Artificial Sequence
  Description of Artificial Sequence Synthetic primer
agaacacgaagtaagataggaaaagg

SEQ ID NO: 341 - DNA, 30 bp, Artificial Sequence
  Description of Artificial Sequence Synthetic primer
cacacaaatcttagtatcattatcttcaaa

SEQ ID NO: 342 - DNA, 28 bp, Artificial Sequence
  Description of Artificial Sequence Synthetic primer
caaatcttgttgtgaaatgttgtaatgt

SEQ ID NO: 343 - DNA, 22 bp, Artificial Sequence
  Description of Artificial Sequence Synthetic primer
tgtcaacaacaaaaggttgagg

SEQ ID NO: 344 - DNA, 26 bp, Artificial Sequence
  Description of Artificial Sequence Synthetic primer
tcaaaatgataagacaatcccaaacg

SEQ ID NO: 345 - DNA, 23 bp, Artificial Sequence
  Description of Artificial Sequence Synthetic primer
gaacaccatgaagacgatgatcc

SEQ ID NO: 346 - DNA, 20 bp, Artificial Sequence
  Description of Artificial Sequence Synthetic primer
tgctgacttcactaagctcc

SEQ ID NO: 347 - DNA, 23 bp, Artificial Sequence
  Description of Artificial Sequence Synthetic primer
tgcttggtaagtgatacccttgg

SEQ ID NO: 348 - DNA, 19 bp, Artificial Sequence
  Description of Artificial Sequence Synthetic primer
agggcctctctagggctcg

SEQ ID NO: 349 - DNA, 25 bp, Artificial Sequence
  Description of Artificial Sequence Synthetic primer
tgcaagagataggattgtggaattg

SEQ ID NO: 350 - DNA, 25 bp, Artificial Sequence
  Description of Artificial Sequence Synthetic primer
actcaagttcttcaagtgtaccata

SEQ ID NO: 351 - DNA, 25 bp, Artificial Sequence
  Description of Artificial Sequence Synthetic primer
ttgttatgaagagtttgagcaactg

SEQ ID NO: 352 - DNA, 29 bp, Artificial Sequence
  Description of Artificial Sequence Synthetic primer
actctgtatgtttcttctttttctagcag

SEQ ID NO: 353 - DNA, 23 bp, Artificial Sequence
  Description of Artificial Sequence Synthetic primer
tttgaatgaagctcgatgtttgc

SEQ ID NO: 354 - DNA, 28 bp, Artificial Sequence
  Description of Artificial Sequence Synthetic primer
ccattagcctacaaatagtaacagatgc

SEQ ID NO: 355 - DNA, 20 bp, Artificial Sequence
  Description of Artificial Sequence Synthetic primer
gcgtttgtgaagaggaagcc

SEQ ID NO: 356 - DNA, 23 bp, Artificial Sequence
  Description of Artificial Sequence Synthetic primer
acccttgaaaagttaagagcacg

SEQ ID NO: 357 - DNA, 27 bp, Artificial Sequence
  Description of Artificial Sequence Synthetic primer
agaaataaatgaagagtttttgaagcc

SEQ ID NO: 358 - DNA, 21 bp, Artificial Sequence
  Description of Artificial Sequence Synthetic primer
tggatgaataagcaaagcagc

SEQ ID NO: 359 - DNA, 24 bp, Artificial Sequence
  Description of Artificial Sequence Synthetic primer
gctcaagtctatgttgaaggaagc

SEQ ID NO: 360 - DNA, 24 bp, Artificial Sequence
  Description of Artificial Sequence Synthetic primer
aacatcctcagaaagcataggtcc

SEQ ID NO: 361 - DNA, 23 bp, Artificial Sequence
  Description of Artificial Sequence Synthetic primer
tcaatggttgaaatacgagaccg

SEQ ID NO: 362 - DNA, 19 bp, Artificial Sequence
  Description of Artificial Sequence Synthetic primer
atcgttgagaagtcgtccg

SEQ ID NO: 363 - DNA, 19 bp, Artificial Sequence
  Description of Artificial Sequence Synthetic primer
cgcgtggttttggctctgg

SEQ ID NO: 364 - DNA, 21 bp, Artificial Sequence
  Description of Artificial Sequence Synthetic primer
ggtgaatccgtcgtcattggc

SEQ ID NO: 365 - DNA, 22 bp, Artificial Sequence
  Description of Artificial Sequence Synthetic primer
tgcattccaacattaggtgctc

SEQ ID NO: 366 - DNA, 23 bp, Artificial Sequence
  Description of Artificial Sequence Synthetic primer
tgcttttccttgcaccatttagt

SEQ ID NO: 367 - DNA, 20 bp, Artificial Sequence
  Description of Artificial Sequence Synthetic primer
ttatgacactgcctgggtgg

SEQ ID NO: 368 - DNA, 20 bp, Artificial Sequence
  Description of Artificial Sequence Synthetic primer
gaggaagaccccatgaaccg

SEQ ID NO: 369 - DNA, 21 bp, Artificial Sequence
  Description of Artificial Sequence Synthetic primer
agagaatcagcactctgacgg

SEQ ID NO: 370 - DNA, 19 bp, Artificial Sequence
  Description of Artificial Sequence Synthetic primer
caccccatcgcttcattgc

SEQ ID NO: 371 - DNA, 20 bp, Artificial Sequence
  Description of Artificial Sequence Synthetic primer
tcaggaagctactcagaggg

SEQ ID NO: 372 - DNA, 23 bp, Artificial Sequence
  Description of Artificial Sequence Synthetic primer
acagagaaccatttttcctttgg

SEQ ID NO: 373 - DNA, 22 bp, Artificial Sequence
  Description of Artificial Sequence Synthetic primer
tatgctcgtctctcaatggtgg

SEQ ID NO: 374 - DNA, 21 bp, Artificial Sequence
  Description of Artificial Sequence Synthetic primer
agaacacactcctctccttgc

SEQ ID NO: 375 - DNA, 21 bp, Artificial Sequence
  Description of Artificial Sequence Synthetic primer
tccgagatcatcatacaccca

SEQ ID NO: 376 - DNA, 22 bp, Artificial Sequence
  Description of Artificial Sequence Synthetic primer
tctcccctagttgaaatgctgt

SEQ ID NO: 377 - DNA, 30 bp, Artificial Sequence
  Description of Artificial Sequence Synthetic primer
gaaaacttcatattgttcctcaaatgttcg

SEQ ID NO: 378 - DNA, 25 bp, Artificial Sequence
  Description of Artificial Sequence Synthetic primer
gagttcttcacggtgtattgattgg

SEQ ID NO: 379 - DNA, 23 bp, Artificial Sequence
  Description of Artificial Sequence Synthetic primer
gtgggttgtagagaataggttgg

SEQ ID NO: 380 - DNA, 21 bp, Artificial Sequence
  Description of Artificial Sequence Synthetic primer
gcgggcatcagataattcagg

SEQ ID NO: 381 - DNA, 21 bp, Artificial Sequence
  Description of Artificial Sequence Synthetic primer
cctgaattatctgatgcccgc

SEQ ID NO: 382 - DNA, 22 bp, Artificial Sequence
  Description of Artificial Sequence Synthetic primer
ctagttcctcttctgaacctcc

SEQ ID NO: 383 - DNA, 23 bp, Artificial Sequence
  Description of Artificial Sequence Synthetic primer
cagtgttgattgtttgtcggagc

SEQ ID NO: 384 - DNA, 20 bp, Artificial Sequence
  Description of Artificial Sequence Synthetic primer
ttgtcacactacgtccttgc

SEQ ID NO: 385 - DNA, 25 bp, Artificial Sequence
  Description of Artificial Sequence Synthetic primer
aagatcagccaactatgatcctccc

SEQ ID NO: 386 - DNA, 30 bp, Artificial Sequence
  Description of Artificial Sequence Synthetic primer
atggaagagactgaatgaaatcaaaagacc

SEQ ID NO: 387 - DNA, 25 bp, Artificial Sequence
  Description of Artificial Sequence Synthetic primer
ttagaagatcagccaactatgatcc

SEQ ID NO: 388 - DNA, 25 bp, Artificial Sequence
  Description of Artificial Sequence Synthetic primer
gaatgaaatcaaaagaccaaatggg

SEQ ID NO: 389 - DNA, 29 bp, Artificial Sequence
  Description of Artificial Sequence Synthetic primer
aaagaagaagtgaaaaagatgttagttgg

SEQ ID NO: 390 - DNA, 25 bp, Artificial Sequence
  Description of Artificial Sequence Synthetic primer
aaaagcctaaattcgagagcagtgg

SEQ ID NO: 391 - DNA, 21 bp, Artificial Sequence
  Description of Artificial Sequence Synthetic primer
gagacgggaaagttcaaagcg

SEQ ID NO: 392 - DNA, 20 bp, Artificial Sequence
  Description of Artificial Sequence Synthetic primer
tgaagtggcatctccaaggc

SEQ ID NO: 393 - DNA, 25 bp, Artificial Sequence
  Description of Artificial Sequence Synthetic primer
gcttcattctatgtgaaaaatggcg

SEQ ID NO: 394 - DNA, 20 bp, Artificial Sequence
  Description of Artificial Sequence Synthetic primer
cgcaaattcaagcaaagtgc

SEQ ID NO: 395 - DNA, 27 bp, Artificial Sequence
  Description of Artificial Sequence Synthetic primer
aaatggtttatgctagagatagattgg

SEQ ID NO: 396 - DNA, 25 bp, Artificial Sequence
  Description of Artificial Sequence Synthetic primer
cttgcagatattctcctaaagtggc

SEQ ID NO: 397 - DNA, 20 bp, Artificial Sequence
  Description of Artificial Sequence Synthetic primer
agaggcttttctatggcagg

SEQ ID NO: 398 - DNA, 24 bp, Artificial Sequence
  Description of Artificial Sequence Synthetic primer
aagctctaattcttccaatgttcc

SEQ ID NO: 399 - DNA, 27 bp, Artificial Sequence
  Description of Artificial Sequence Synthetic primer
tgcctttctttactttatttaacaccg

SEQ ID NO: 400 - DNA, 23 bp, Artificial Sequence
  Description of Artificial Sequence Synthetic primer
gctcttctaacacatcatacgcc

SEQ ID NO: 401 - DNA, 20 bp, Artificial Sequence
  Description of Artificial Sequence Synthetic primer
acaccgtaaatgaaatggcg

SEQ ID NO: 402 - DNA, 23 bp, Artificial Sequence
  Description of Artificial Sequence Synthetic primer
cgagttcttgaggtattcaacgc

SEQ ID NO: 403 - DNA, 21 bp, Artificial Sequence
  Description of Artificial Sequence Synthetic primer
tgggcagagttatgtagatgc

SEQ ID NO: 404 - DNA, 19 bp, Artificial Sequence
  Description of Artificial Sequence Synthetic primer
tcctattgaaagcgaggcg

SEQ ID NO: 405 - DNA, 19 bp, Artificial Sequence
  Description of Artificial Sequence Synthetic primer
acgcctcgctttcaatagg

SEQ ID NO: 406 - DNA, 21 bp, Artificial Sequence
  Description of Artificial Sequence Synthetic primer
ctgcaagtcgtaacatcaagg

SEQ ID NO: 407 - DNA, 26 bp, Artificial Sequence
  Description of Artificial Sequence Synthetic primer
tggaagaggcaaaatggttttatagc

SEQ ID NO: 408 - DNA, 27 bp, Artificial Sequence
  Description of Artificial Sequence Synthetic primer
gatcatctgcaagtcgtaacatcaagg

SEQ ID NO: 409 - DNA, 24 bp, Artificial Sequence
  Description of Artificial Sequence Synthetic primer
aaagaggtgacattcttaaatcgg

SEQ ID NO: 410 - DNA, 29 bp, Artificial Sequence
  Description of Artificial Sequence Synthetic primer
agttatattcatcttcatcattcatctcc

SEQ ID NO: 411 - DNA, 23 bp, Artificial Sequence
  Description of Artificial Sequence Synthetic primer
ggtgtttctgaagatgaagctcg

SEQ ID NO: 412 - DNA, 24 bp, Artificial Sequence
  Description of Artificial Sequence Synthetic primer
ctgaaatacgtttccttgaatggc

SEQ ID NO: 413 - DNA, 26 bp, Artificial Sequence
  Description of Artificial Sequence Synthetic primer
tctcgaagatcagcaaactatcaacc

SEQ ID NO: 414 - DNA, 24 bp, Artificial Sequence
  Description of Artificial Sequence Synthetic primer
acataatcaaattgccaaagtggg

SEQ ID NO: 415 - DNA, 27 bp, Artificial Sequence
  Description of Artificial Sequence Synthetic primer
agacttggaatctcttatcactttgag

SEQ ID NO: 416 - DNA, 27 bp, Artificial Sequence
  Description of Artificial Sequence Synthetic primer
ggcatacacattatttttgttggtgtt

SEQ ID NO: 417 - DNA, 26 bp, Artificial Sequence
  Description of Artificial Sequence Synthetic primer
gtacaacaccaacaaaaataatgtgt

SEQ ID NO: 418 - DNA, 25 bp, Artificial Sequence
  Description of Artificial Sequence Synthetic primer
tcgtaggagtctaaattcaagagaa

SEQ ID NO: 419 - DNA, 23 bp, Artificial Sequence
  Description of Artificial Sequence Synthetic primer
gaggcaattttatggtgtgtacc

SEQ ID NO: 420 - DNA, 23 bp, Artificial Sequence
  Description of Artificial Sequence Synthetic primer
ttgttttgtgtcttgtctcttcc

SEQ ID NO: 421 - DNA, 20 bp, Artificial Sequence
  Description of Artificial Sequence Synthetic primer
tgggaaagcactgatatggg

SEQ ID NO: 422 - DNA, 23 bp, Artificial Sequence
  Description of Artificial Sequence Synthetic primer
agctccaattcatctagtgtacc

SEQ ID NO: 423 - DNA, 23 bp, Artificial Sequence
  Description of Artificial Sequence Synthetic primer
tgggacattagtgctatggatgg

SEQ ID NO: 424 - DNA, 27 bp, Artificial Sequence
  Description of Artificial Sequence Synthetic primer
tctgaaggaattttattatatggaggc

SEQ ID NO: 425 - DNA, 26 bp, Artificial Sequence
  Description of Artificial Sequence Synthetic primer
agatttgaagagtacattgagaatgc

SEQ ID NO: 426 - DNA, 25 bp, Artificial Sequence
  Description of Artificial Sequence Synthetic primer
cgatgaatgtcgtattatggtaggg

SEQ ID NO: 427 - DNA, 21 bp, Artificial Sequence
  Description of Artificial Sequence Synthetic primer
agaggaagctcgtcaacgtat

SEQ ID NO: 428 - DNA, 21 bp, Artificial Sequence
  Description of Artificial Sequence Synthetic primer
atgtgccattctaccaaggtt

SEQ ID NO: 429 - DNA, 25 bp, Artificial Sequence
  Description of Artificial Sequence Synthetic primer
gacttggaatctcttaccactttga

SEQ ID NO: 430 - DNA, 25 bp, Artificial Sequence
  Description of Artificial Sequence Synthetic primer
accatgttgtcttaggagtctaaat

SEQ ID NO: 431 - DNA, 25 bp, Artificial Sequence
  Description of Artificial Sequence Synthetic primer
gaatagttctcgaagatcagcaaac

SEQ ID NO: 432 - DNA, 20 bp, Artificial Sequence
  Description of Artificial Sequence Synthetic primer
caaattgccaaagtgggggt

SEQ ID NO: 433 - DNA, 24 bp, Artificial Sequence
  Description of Artificial Sequence Synthetic primer
gcaaagacttggaatctcttacca

SEQ ID NO: 434 - DNA, 22 bp, Artificial Sequence
  Description of Artificial Sequence Synthetic primer
gagacaccggataaccatgttg

SEQ ID NO: 435 - DNA, 23 bp, Artificial Sequence
  Description of Artificial Sequence Synthetic primer
gaggcaattttatggtgtcttcc

SEQ ID NO: 436 - DNA, 22 bp, Artificial Sequence
  Description of Artificial Sequence Synthetic primer
tgttgtgtgtcttgtctcttcc

SEQ ID NO: 437 - DNA, 20 bp, Artificial Sequence
  Description of Artificial Sequence Synthetic primer
gtgggaaagcactggtatgg

SEQ ID NO: 438 - DNA, 23 bp, Artificial Sequence
  Description of Artificial Sequence Synthetic primer
agctccaattcatctagtgtacc

SEQ ID NO: 439 - DNA, 21 bp, Artificial Sequence
  Description of Artificial Sequence Synthetic primer
ttagtgctatggatgggctcc

SEQ ID NO: 440 - DNA, 28 bp, Artificial Sequence
  Description of Artificial Sequence Synthetic primer
cgatttctgaaggacttttattatatgg

SEQ ID NO: 441 - DNA, 26 bp, Artificial Sequence
  Description of Artificial Sequence Synthetic primer
agagaagcaagatggtattatgatgg

SEQ ID NO: 442 - DNA, 25 bp, Artificial Sequence
  Description of Artificial Sequence Synthetic primer
cgatgaatgtcgtattatggtaggg

SEQ ID NO: 443 - DNA, 21 bp, Artificial Sequence
  Description of Artificial Sequence Synthetic primer
tggcgtgtctgaagaagaagc

SEQ ID NO: 444 - DNA, 23 bp, Artificial Sequence
  Description of Artificial Sequence Synthetic primer
aacattggagagtagtcatcacc

SEQ ID NO: 445 - DNA, 23 bp, Artificial Sequence
  Description of Artificial Sequence Synthetic primer
catgatggtgatgactactctcc

SEQ ID NO: 446 - DNA, 23 bp, Artificial Sequence
  Description of Artificial Sequence Synthetic primer
agtacatgatcttttgtttggcg

SEQ ID NO: 447 - DNA, 22 bp, Artificial Sequence
  Description of Artificial Sequence Synthetic primer
tgcaaagaagaacgagtgaagg

SEQ ID NO: 448 - DNA, 30 bp, Artificial Sequence
  Description of Artificial Sequence Synthetic primer
tgtttgtagtagtttagatcatgtttttcc

SEQ ID NO: 449 - DNA, 22 bp, Artificial Sequence
  Description of Artificial Sequence Synthetic primer
ataaccgatgtttcgggtttgc

SEQ ID NO: 450 - DNA, 25 bp, Artificial Sequence
  Description of Artificial Sequence Synthetic primer
cctctctatggtctttcttaatggc

SEQ ID NO: 451 - DNA, 25 bp, Artificial Sequence
  Description of Artificial Sequence Synthetic primer
gaaagctaacaaccaaaatctctgc

SEQ ID NO: 452 - DNA, 22 bp, Artificial Sequence
  Description of Artificial Sequence Synthetic primer
ttgcatggcttgggtaagaagc

SEQ ID NO: 453 - DNA, 23 bp, Artificial Sequence
  Description of Artificial Sequence Synthetic primer
agaagaagctcgatggttgaacg

SEQ ID NO: 454 - DNA, 23 bp, Artificial Sequence
  Description of Artificial Sequence Synthetic primer
catgaacctagcaagtagagtgg

SEQ ID NO: 455 - DNA, 21 bp, Artificial Sequence
  Description of Artificial Sequence Synthetic primer
gccaactgttatgcctttccc

SEQ ID NO: 456 - DNA, 26 bp, Artificial Sequence
  Description of Artificial Sequence Synthetic primer
gggattggatctataagtaaagcagc

SEQ ID NO: 457 - DNA, 21 bp, Artificial Sequence
  Description of Artificial Sequence Synthetic primer
atgctaccccatccaatgtgc

SEQ ID NO: 458 - DNA, 28 bp, Artificial Sequence
  Description of Artificial Sequence Synthetic primer
aatataatcgaaagaccaaatggagggc

SEQ ID NO: 459 - DNA, 27 bp, Artificial Sequence
  Description of Artificial Sequence Synthetic primer
attagttgagatggaaaactctttagc

SEQ ID NO: 460 - DNA, 23 bp, Artificial Sequence
  Description of Artificial Sequence Synthetic primer
atagccatgttgacgtagaagcc

SEQ ID NO: 461 - DNA, 20 bp, Artificial Sequence
  Description of Artificial Sequence Synthetic primer
agctcccacttcatcggagg

SEQ ID NO: 462 - DNA, 24 bp, Artificial Sequence
  Description of Artificial Sequence Synthetic primer
tttggctaactcaagcaacatagg

SEQ ID NO: 463 - DNA, 28 bp, Artificial Sequence
  Description of Artificial Sequence Synthetic primer
agacatactaaacttggagagaaattga

SEQ ID NO: 464 - DNA, 28 bp, Artificial Sequence
  Description of Artificial Sequence Synthetic primer
tattccataagaaacattccatcaatcg

SEQ ID NO: 465 - DNA, 24 bp, Artificial Sequence
  Description of Artificial Sequence Synthetic primer
ggagacatactaaacttggagaga

SEQ ID NO: 466 - DNA, 25 bp, Artificial Sequence
  Description of Artificial Sequence Synthetic primer
gctttggtgaaaagctctaattcat

SEQ ID NO: 467 - DNA, 25 bp, Artificial Sequence
  Description of Artificial Sequence Synthetic primer
agttaccagaatacatgaagatgcc

SEQ ID NO: 468 - DNA, 24 bp, Artificial Sequence
  Description of Artificial Sequence Synthetic primer
tgaatgttgatggagatttcttgg

SEQ ID NO: 469 - DNA, 24 bp, Artificial Sequence
  Description of Artificial Sequence Synthetic primer
aaatggttggatttcagtaggagc

SEQ ID NO: 470 - DNA, 30 bp, Artificial Sequence
  Description of Artificial Sequence Synthetic primer
gaaaatctctttagtatttgtaattgtgcc

SEQ ID NO: 471 - DNA, 23 bp, Artificial Sequence
  Description of Artificial Sequence Synthetic primer
aattgaaaagaggtgatgctccg

SEQ ID NO: 472 - DNA, 30 bp, Artificial Sequence
  Description of Artificial Sequence Synthetic primer
tttggattgattatcttgagaactatgacc

SEQ ID NO: 473 - DNA, 22 bp, Artificial Sequence
  Description of Artificial Sequence Synthetic primer
aaagatcaaccagcagcaatcg

SEQ ID NO: 474 - DNA, 23 bp, Artificial Sequence
  Description of Artificial Sequence Synthetic primer
gtgggtttgtagtttcctgatcg

SEQ ID NO: 475 - DNA, 28 bp, Artificial Sequence
  Description of Artificial Sequence Synthetic primer
aaggagagtgaaaatcctttagttaagc

SEQ ID NO: 476 - DNA, 24 bp, Artificial Sequence
  Description of Artificial Sequence Synthetic primer
gagtttgaaatgaagagcagtggc

SEQ ID NO: 477 - DNA, 25 bp, Artificial Sequence
  Description of Artificial Sequence Synthetic primer
aatgccttcaaaaacgagcaaaagg

SEQ ID NO: 478 - DNA, 28 bp, Artificial Sequence
  Description of Artificial Sequence Synthetic primer
gtcatcaagtattgtttgagatgtttgg

SEQ ID NO: 479 - DNA, 21 bp, Artificial Sequence
  Description of Artificial Sequence Synthetic primer
tggcattgaaatatgaggcgg

SEQ ID NO: 480 - DNA, 22 bp, Artificial Sequence
  Description of Artificial Sequence Synthetic primer
gctgaagctcatctagtgtacc

SEQ ID NO: 481 - DNA, 25 bp, Artificial Sequence
  Description of Artificial Sequence Synthetic primer
ataaatgaactggatcagctacccg

SEQ ID NO: 482 - DNA, 28 bp, Artificial Sequence
  Description of Artificial Sequence Synthetic primer
gatggtgtgaatcccattttctttgagg

SEQ ID NO: 483 - DNA, 21 bp, Artificial Sequence
  Description of Artificial Sequence Synthetic primer
ttgggggatctgtgtaaatgc

SEQ ID NO: 484 - DNA, 24 bp, Artificial Sequence
  Description of Artificial Sequence Synthetic primer
cagttcctgagacacgtaaaatgg

SEQ ID NO: 485 - DNA, 20 bp, Artificial Sequence
  Description of Artificial Sequence Synthetic primer
gcagtgcatggcttttcacc

SEQ ID NO: 486 - DNA, 22 bp, Artificial Sequence
  Description of Artificial Sequence Synthetic primer
tccaaatagggagggatgatga

SEQ ID NO: 487 - DNA, 24 bp, Artificial Sequence
  Description of Artificial Sequence Synthetic primer
acaatgtactgtggtcgataaccc

SEQ ID NO: 488 - DNA, 27 bp, Artificial Sequence
  Description of Artificial Sequence Synthetic primer
aatgaaatcaaaagaccaaatgggagg

SEQ ID NO: 489 - DNA, 25 bp, Artificial Sequence
  Description of Artificial Sequence Synthetic primer
gagaaagacgtgaaaaggatactgg

SEQ ID NO: 490 - DNA, 24 bp, Artificial Sequence
  Description of Artificial Sequence Synthetic primer
tcaaatccatattggcgtagaagc

SEQ ID NO: 491 - DNA, 24 bp, Artificial Sequence
  Description of Artificial Sequence Synthetic primer
agcttcattctatgggaaaaaggg

SEQ ID NO: 492 - DNA, 21 bp, Artificial Sequence
  Description of Artificial Sequence Synthetic primer
tgaaccacctagcttccaacc

SEQ ID NO: 493 - DNA, 29 bp, Artificial Sequence
  Description of Artificial Sequence Synthetic primer
gcaatctaaacttggagaaaagaaaatgg

SEQ ID NO: 494 - DNA, 24 bp, Artificial Sequence
  Description of Artificial Sequence Synthetic primer
tttgtgaaaagctctaattcctcc

SEQ ID NO: 495 - DNA, 27 bp, Artificial Sequence
  Description of Artificial Sequence Synthetic primer
catgcttattttgctttcacaaatccc

SEQ ID NO: 496 - DNA, 29 bp, Artificial Sequence
  Description of Artificial Sequence Synthetic primer
taggtcatcttcaagtcgtaaaagtatgg

SEQ ID NO: 497 - DNA, 24 bp, Artificial Sequence
  Description of Artificial Sequence Synthetic primer
gagatgaaaagaggagatgttccg

SEQ ID NO: 498 - DNA, 23 bp, Artificial Sequence
  Description of Artificial Sequence Synthetic primer
aagcatgtccatcaccataaagg

SEQ ID NO: 499 - DNA, 30 bp, Artificial Sequence
  Description of Artificial Sequence Synthetic primer
aaacatggaaagagatgaataaagaaatgg

SEQ ID NO: 500 - DNA, 26 bp, Artificial Sequence
  Description of Artificial Sequence Synthetic primer
gagaagcatgtccatcaccataaagg

SEQ ID NO: 501 - DNA, 21 bp, Artificial Sequence
  Description of Artificial Sequence Synthetic primer
attcaaagaacaaccggaagc

SEQ ID NO: 502 - DNA, 21 bp, Artificial Sequence
  Description of Artificial Sequence Synthetic primer
aaaaatgagcatcccacaacg

SEQ ID NO: 503 - DNA, 27 bp, Artificial Sequence
  Description of Artificial Sequence Synthetic primer
aaagctatgggaaaagaatcaatgagc

SEQ ID NO: 504 - DNA, 24 bp, Artificial Sequence
  Description of Artificial Sequence Synthetic primer
ggagtttgaaatgaagagcagtgg

SEQ ID NO: 505 - DNA, 26 bp, Artificial Sequence
  Description of Artificial Sequence Synthetic primer
gaggcttcattctattcatttagggg

SEQ ID NO: 506 - DNA, 25 bp, Artificial Sequence
  Description of Artificial Sequence Synthetic primer
tcaacagatttagtttgacattgcc

SEQ ID NO: 507 - DNA, 23 bp, Artificial Sequence
  Description of Artificial Sequence Synthetic primer
tctaggttgacagaaaggctacc

SEQ ID NO: 508 - DNA, 24 bp, Artificial Sequence
  Description of Artificial Sequence Synthetic primer
gtccatgagtgttagcaagagacc

SEQ ID NO: 509 - DNA, 26 bp, Artificial Sequence
  Description of Artificial Sequence Synthetic primer
tataaatgaattggatcagctacccg

SEQ ID NO: 510 - DNA, 30 bp, Artificial Sequence
  Description of Artificial Sequence Synthetic primer
tataaaaagcaacaaacaatatcttcatgt

SEQ ID NO: 511 - DNA, 22 bp, Artificial Sequence
  Description of Artificial Sequence Synthetic primer
gttgggggatctgtgtaaatgc

SEQ ID NO: 512 - DNA, 25 bp, Artificial Sequence
  Description of Artificial Sequence Synthetic primer
agttggatattgaagcaagtgttcc

SEQ ID NO: 513 - DNA, 25 bp, Artificial Sequence
  Description of Artificial Sequence Synthetic primer
tgtgtaaatgctatatggaggaggc

SEQ ID NO: 514 - DNA, 24 bp, Artificial Sequence
  Description of Artificial Sequence Synthetic primer
tcgaaagacagttcctgagacacg

SEQ ID NO: 515 - DNA, 20 bp, Artificial Sequence
  Description of Artificial Sequence Synthetic primer
tgctacatgcgtgaaaaggg

SEQ ID NO: 516 - DNA, 19 bp, Artificial Sequence
  Description of Artificial Sequence Synthetic primer
agtaccaaagccatcaccc

SEQ ID NO: 517 - DNA, 27 bp, Artificial Sequence
  Description of Artificial Sequence Synthetic primer
ttaatatcatcatcactaccttgcatt

SEQ ID NO: 518 - DNA, 26 bp, Artificial Sequence
  Description of Artificial Sequence Synthetic primer
ggactaatagatgtttttggtgtgaa

SEQ ID NO: 519 - DNA, 28 bp, Artificial Sequence
  Description of Artificial Sequence Synthetic primer
caatgtactgtggtcaataatagtagcc

SEQ ID NO: 520 - DNA, 22 bp, Artificial Sequence
  Description of Artificial Sequence Synthetic primer
aatcgaaagaccaaatcggagg

SEQ ID NO: 521 - DNA, 24 bp, Artificial Sequence
  Description of Artificial Sequence Synthetic primer
ataagggagaatcctatacaaggc

SEQ ID NO: 522 - DNA, 22 bp, Artificial Sequence
  Description of Artificial Sequence Synthetic primer
cctaaattcgagagcaatgggg

SEQ ID NO: 523 - DNA, 22 bp, Artificial Sequence
  Description of Artificial Sequence Synthetic primer
agcatattggaggaagctaggg

SEQ ID NO: 524 - DNA, 21 bp, Artificial Sequence
  Description of Artificial Sequence Synthetic primer
tcgataaaccacttggcctcc

SEQ ID NO: 525 - DNA, 27 bp, Artificial Sequence
  Description of Artificial Sequence Synthetic primer
gatctaaaacatttgtctaggtggtgg

SEQ ID NO: 526 - DNA, 26 bp, Artificial Sequence
  Description of Artificial Sequence Synthetic primer
attctcctaaagtagctgaaatctgg

SEQ ID NO: 527 - DNA, 20 bp, Artificial Sequence
  Description of Artificial Sequence Synthetic primer
tcgtgttgttgagagatggg

SEQ ID NO: 528 - DNA, 30 bp, Artificial Sequence
  Description of Artificial Sequence Synthetic primer
aagtgtttttcttttaatacatctagtgcc

SEQ ID NO: 529 - DNA, 21 bp, Artificial Sequence
  Description of Artificial Sequence Synthetic primer
tggttggatttcagtaggagc

SEQ ID NO: 530 - DNA, 20 bp, Artificial Sequence
  Description of Artificial Sequence Synthetic primer
acgatcatggcagattgacg

SEQ ID NO: 531 - DNA, 26 bp, Artificial Sequence
  Description of Artificial Sequence Synthetic primer
cataacgtatctaaagaggaagctcg

SEQ ID NO: 532 - DNA, 25 bp, Artificial Sequence
  Description of Artificial Sequence Synthetic primer
tgcaaagtttttggcatcatcaacc

SEQ ID NO: 533 - DNA, 22 bp, Artificial Sequence
  Description of Artificial Sequence Synthetic primer
aaagatcaaccagcagcaatcg

SEQ ID NO: 534 - DNA, 23 bp, Artificial Sequence
  Description of Artificial Sequence Synthetic primer
gtgggtttgtagtttcctgatcg

SEQ ID NO: 535 - DNA, 26 bp, Artificial Sequence
  Description of Artificial Sequence Synthetic primer
aagctatgggaaaagaatcaatgagc

SEQ ID NO: 536 - DNA, 23 bp, Artificial Sequence
  Description of Artificial Sequence Synthetic primer
gagtttgaaatgaagagcagtgg

SEQ ID NO: 537 - DNA, 24 bp, Artificial Sequence
  Description of Artificial Sequence Synthetic primer
aggctgaaaatcctttagttaagc

SEQ ID NO: 538 - DNA, 24 bp, Artificial Sequence
  Description of Artificial Sequence Synthetic primer
ggactgaatccatattgtcgaagg

SEQ ID NO: 539 - DNA, 23 bp, Artificial Sequence
  Description of Artificial Sequence Synthetic primer
tgccttcaaaaacgagaaaaagg

SEQ ID NO: 540 - DNA, 25 bp, Artificial Sequence
  Description of Artificial Sequence Synthetic primer
agtctcttgcttcatctaatatggg

SEQ ID NO: 541 - DNA, 23 bp, Artificial Sequence
  Description of Artificial Sequence Synthetic primer
tgcagtggcattgaaatatgagg

SEQ ID NO: 542 - DNA, 22 bp, Artificial Sequence
  Description of Artificial Sequence Synthetic primer
gctgaagctcatctagtgtacc

SEQ ID NO: 543 - DNA, 25 bp, Artificial Sequence
  Description of Artificial Sequence Synthetic primer
ataaatgaactggatcagctacccg

SEQ ID NO: 544 - DNA, 30 bp, Artificial Sequence; identical to SEQ ID NO: 510
  Description of Artificial Sequence Synthetic primer

SEQ ID NO: 545 - DNA, 24 bp, Artificial Sequence
  Description of Artificial Sequence Synthetic primer
tgctatatggaggaggcaaaatgg

SEQ ID NO: 546 - DNA, 24 bp, Artificial Sequence
  Description of Artificial Sequence Synthetic primer
tcgaaaaacagttcctgagacacg

SEQ ID NO: 547 - DNA, 20 bp, Artificial Sequence
  Description of Artificial Sequence Synthetic primer
tgctacatgcgtgaaaaggg

SEQ ID NO: 548 - DNA, 22 bp, Artificial Sequence
  Description of Artificial Sequence Synthetic primer
tgtggtacatctccattgctcc

SEQ ID NO: 549 - DNA, 22 bp, Artificial Sequence
  Description of Artificial Sequence Synthetic primer
tggcttttcaccaatttgctcc

SEQ ID NO: 550 - DNA, 22 bp, Artificial Sequence
  Description of Artificial Sequence Synthetic primer
aaatgggaggtccatagttggc

SEQ ID NO: 551 - DNA, 25 bp, Artificial Sequence
  Description of Artificial Sequence Synthetic primer
ttgaagaaagaagtgacaagatggc

SEQ ID NO: 552 - DNA, 21 bp, Artificial Sequence
  Description of Artificial Sequence Synthetic primer
ggcgtagaagcctaaattcgc

SEQ ID NO: 553 - DNA, 24 bp, Artificial Sequence
  Description of Artificial Sequence Synthetic primer
acatggagaaaatggagaatgagg

SEQ ID NO: 554 - DNA, 20 bp, Artificial Sequence
  Description of Artificial Sequence Synthetic primer
cgcaacctcgaaacaagtcg

SEQ ID NO: 555 - DNA, 21 bp, Artificial Sequence
  Description of Artificial Sequence Synthetic primer
aggggattgtaaacttggtgg

SEQ ID NO: 556 - DNA, 28 bp, Artificial Sequence
  Description of Artificial Sequence Synthetic primer
ttctctaaaatagctgaaattctcctcg

SEQ ID NO: 557 - DNA, 27 bp, Artificial Sequence
  Description of Artificial Sequence Synthetic primer
tgagttaccagattacatgaagatgcc

SEQ ID NO: 558 - DNA, 24 bp, Artificial Sequence
  Description of Artificial Sequence Synthetic primer
actaatacatcgaaccccatctca

SEQ ID NO: 559 - DNA, 27 bp, Artificial Sequence
  Description of Artificial Sequence Synthetic primer
acaagaagcaaaatggtattatagtgg

SEQ ID NO: 560 - DNA, 30 bp, Artificial Sequence
  Description of Artificial Sequence Synthetic primer
ttttgttataggatttgtgaaacaataagc

SEQ ID NO: 561 - DNA, 22 bp, Artificial Sequence
  Description of Artificial Sequence Synthetic primer
ataatgctaccgaagacgaagc

SEQ ID NO: 562 - DNA, 22 bp, Artificial Sequence
  Description of Artificial Sequence Synthetic primer
tctgagaaccatgtccatctcc

SEQ ID NO: 563 - DNA, 26 bp, Artificial Sequence
  Description of Artificial Sequence Synthetic primer
aatcacttttgtagatttttcacacc

SEQ ID NO: 564 - DNA, 24 bp, Artificial Sequence
  Description of Artificial Sequence Synthetic primer
gtaatcgaaagaccaaatcggagg

SEQ ID NO: 565 - DNA, 24 bp, Artificial Sequence
  Description of Artificial Sequence Synthetic primer
agaaagaagtgacaagaatgctcc

SEQ ID NO: 566 - DNA, 20 bp, Artificial Sequence
  Description of Artificial Sequence Synthetic primer
agccatgttggcgtagaagc

SEQ ID NO: 567 - DNA, 22 bp, Artificial Sequence
  Description of Artificial Sequence Synthetic primer
tgctttcaaggataagagaggg

SEQ ID NO: 568 - DNA, 24 bp, Artificial Sequence
  Description of Artificial Sequence Synthetic primer
tcttcctcattctccattttctcc

SEQ ID NO: 569 - DNA, 27 bp, Artificial Sequence
  Description of Artificial Sequence Synthetic primer
aatacatggagaaaatggagaatgagg

SEQ ID NO: 570 - DNA, 20 bp, Artificial Sequence
  Description of Artificial Sequence Synthetic primer
cgcaaactcgaacaaagtcg

SEQ ID NO: 571 - DNA, 26 bp, Artificial Sequence
  Description of Artificial Sequence Synthetic primer
tttgctagagatagattgatggaagc

SEQ ID NO: 572 - DNA, 26 bp, Artificial Sequence
  Description of Artificial Sequence Synthetic primer
aaaagctctaattcctccaaagttcc

SEQ ID NO: 573 - DNA, 27 bp, Artificial Sequence
  Description of Artificial Sequence Synthetic primer
atcaatgagttaccagattacatgaag

SEQ ID NO: 574 - DNA, 25 bp, Artificial Sequence
  Description of Artificial Sequence Synthetic primer
actaatacatcgaaccccatctcat

SEQ ID NO: 575 - DNA, 20 bp, Artificial Sequence
  Description of Artificial Sequence Synthetic primer
atacactgagttgggttggc

SEQ ID NO: 576 - DNA, 23 bp, Artificial Sequence
  Description of Artificial Sequence Synthetic primer
tgttcctaaatcatctgcaagcc

SEQ ID NO: 577 - DNA, 23 bp, Artificial Sequence
  Description of Artificial Sequence Synthetic primer
gaattgaatagaggcgacgttcc

SEQ ID NO: 578 - DNA, 27 bp, Artificial Sequence
  Description of Artificial Sequence Synthetic primer
atagtgctgttctagccatatttttgc

SEQ ID NO: 579 - DNA, 28 bp, Artificial Sequence
  Description of Artificial Sequence Synthetic primer
caatgtactgtggtcaataatagtagcc

SEQ ID NO: 580 - DNA, 22 bp, Artificial Sequence
  Description of Artificial Sequence Synthetic primer
aatcgaaagaccaaatcggagg

SEQ ID NO: 581 - DNA, 23 bp, Artificial Sequence
  Description of Artificial Sequence Synthetic primer
actgtggtcaataatagtagccc

SEQ ID NO: 582 - DNA, 26 bp, Artificial Sequence
  Description of Artificial Sequence Synthetic primer
tgaatgtaatcgaaagaccaaatcgg

SEQ ID NO: 583 - DNA, 27 bp, Artificial Sequence
  Description of Artificial Sequence Synthetic primer
aatataagggagaatcctatacaaggc

SEQ ID NO: 584 - DNA, 22 bp, Artificial Sequence
  Description of Artificial Sequence Synthetic primer
atagccatgttggcgtagaagc

SEQ ID NO: 585 - DNA, 26 bp, Artificial Sequence
  Description of Artificial Sequence Synthetic primer
tgagaaaaatggtgaaagcatattgg

SEQ ID NO: 586 - DNA, 19 bp, Artificial Sequence
  Description of Artificial Sequence Synthetic primer
tccgttcttgcagtcctcc

SEQ ID NO: 587 - DNA, 20 bp, Artificial Sequence
  Description of Artificial Sequence Synthetic primer
gaggactgcaagaacggagg

SEQ ID NO: 588 - DNA, 26 bp, Artificial Sequence
  Description of Artificial Sequence Synthetic primer
acaaatgttttagatcgtcttgatgc

SEQ ID NO: 589 - DNA, 24 bp, Artificial Sequence
  Description of Artificial Sequence Synthetic primer
tttgtgagagataggttgatgggg

SEQ ID NO: 590 - DNA, 26 bp, Artificial Sequence
  Description of Artificial Sequence Synthetic primer
aaagctctaattcttctaatgttccg

SEQ ID NO: 591 - DNA, 22 bp, Artificial Sequence
  Description of Artificial Sequence Synthetic primer
atggttggatttcagtaggagc

SEQ ID NO: 592 - DNA, 22 bp, Artificial Sequence
  Description of Artificial Sequence Synthetic primer
gtacgatcatggcagattgacg

SEQ ID NO: 593 - DNA, 28 bp, Artificial Sequence
  Description of Artificial Sequence Synthetic primer
atgcaagaccataatgtatctaaagagg

SEQ ID NO: 594 - DNA, 25 bp, Artificial Sequence
  Description of Artificial Sequence Synthetic primer
tgcaaagtttttggcatcatcaacc

SEQ ID NO: 595 - DNA, 30 bp, Artificial Sequence
  Description of Artificial Sequence Synthetic primer
tcaatgtactgtagtcgatagttctaatcc

SEQ ID NO: 596 - DNA, 25 bp, Artificial Sequence
  Description of Artificial Sequence Synthetic primer
gaccaaatggatggttcatagttgc

SEQ ID NO: 597 - DNA, 23 bp, Artificial Sequence
  Description of Artificial Sequence Synthetic primer
tagaagtcgggtgaaagagatcg

SEQ ID NO: 598 - DNA, 25 bp, Artificial Sequence
  Description of Artificial Sequence Synthetic primer
agaagtctaaattcaagagcaatgg

SEQ ID NO: 599 - DNA, 24 bp, Artificial Sequence
  Description of Artificial Sequence Synthetic primer
atagaagtcgggtgaaagagatcg

SEQ ID NO: 600 - DNA, 27 bp, Artificial Sequence
  Description of Artificial Sequence Synthetic primer
aagcgataagatactccaagtctttgc

SEQ ID NO: 601 - DNA, 22 bp, Artificial Sequence
  Description of Artificial Sequence Synthetic primer
acgagacaggaaaattcaaggc

SEQ ID NO: 602 - DNA, 20 bp, Artificial Sequence
  Description of Artificial Sequence Synthetic primer
tcctccaatgaagtgggagc

SEQ ID NO: 603 - DNA, 22 bp, Artificial Sequence
  Description of Artificial Sequence Synthetic primer
gtggaggcatactaaacttggg

SEQ ID NO: 604 - DNA, 26 bp, Artificial Sequence
  Description of Artificial Sequence Synthetic primer
aaaagctctaattcatccaatgttcc

SEQ ID NO: 605 - DNA, 22 bp, Artificial Sequence
  Description of Artificial Sequence Synthetic primer
accagaatacatgaagatgcct

SEQ ID NO: 606 - DNA, 25 bp, Artificial Sequence
  Description of Artificial Sequence Synthetic primer
tgttgatggagatgtctttgtctct

SEQ ID NO: 607 - DNA, 27 bp, Artificial Sequence
  Description of Artificial Sequence Synthetic primer
gtggatagatatgtgtagaggttttct

SEQ ID NO: 608 - DNA, 20 bp, Artificial Sequence
  Description of Artificial Sequence Synthetic primer
aagaactggtgctcccactg

SEQ ID NO: 609 - DNA, 24 bp, Artificial Sequence
  Description of Artificial Sequence Synthetic primer
tgttcggatgaaatgaaaagaggc

SEQ ID NO: 610 - DNA, 25 bp, Artificial Sequence
  Description of Artificial Sequence Synthetic primer
tgtgacgctctaccaagatttttgc

SEQ ID NO: 611 - DNA, 24 bp, Artificial Sequence
  Description of Artificial Sequence Synthetic primer
caaatgttctgtggtccataaccc

SEQ ID NO: 612 - DNA, 26 bp, Artificial Sequence
  Description of Artificial Sequence Synthetic primer
gaccaaatgggaggttcatagtttcc

SEQ ID NO: 613 - DNA, 29 bp, Artificial Sequence
  Description of Artificial Sequence Synthetic primer
gagaaagatgtgaaaatgatgctacttgg

SEQ ID NO: 614 - DNA, 25 bp, Artificial Sequence
  Description of Artificial Sequence Synthetic primer
tgaggtactacaaatccatactccc

SEQ ID NO: 615 - DNA, 25 bp, Artificial Sequence
  Description of Artificial Sequence Synthetic primer
gatgatgaaacaggagagttcaagg

SEQ ID NO: 616 - DNA, 25 bp, Artificial Sequence
  Description of Artificial Sequence Synthetic primer
gacatttggttgtgaaaattcttgc

SEQ ID NO: 617 - DNA, 22 bp, Artificial Sequence
  Description of Artificial Sequence Synthetic primer
tggatcatgctttggaaatgcc

SEQ ID NO: 618 - DNA, 24 bp, Artificial Sequence
  Description of Artificial Sequence Synthetic primer
agctcttcttgatatgttgattgc

SEQ ID NO: 619 - DNA, 23 bp, Artificial Sequence
  Description of Artificial Sequence Synthetic primer
tggtggaagcattctaaacttgg

SEQ ID NO: 620 - DNA, 25 bp, Artificial Sequence
  Description of Artificial Sequence Synthetic primer
agcattagcgaagagttgtagttcc

SEQ ID NO: 621 - DNA, 22 bp, Artificial Sequence
  Description of Artificial Sequence Synthetic primer
agtgtttcatgtggcaagttgg

SEQ ID NO: 622 - DNA, 22 bp, Artificial Sequence
  Description of Artificial Sequence Synthetic primer
agcgaagagttgtagttcctcc

SEQ ID NO: 623 - DNA, 29 bp, Artificial Sequence
  Description of Artificial Sequence Synthetic primer
gggatttgaaagtaatagatgagttaccg

SEQ ID NO: 624 - DNA, 24 bp, Artificial Sequence
  Description of Artificial Sequence Synthetic primer
agaaagccatcttcatgtaatccg

SEQ ID NO: 625 - DNA, 22 bp, Artificial Sequence
  Description of Artificial Sequence Synthetic primer
agtggataccaaccaacattgc

SEQ ID NO: 626 - DNA, 30 bp, Artificial Sequence
  Description of Artificial Sequence Synthetic primer
tgttatgagatttgtaaaacagaaataagc

SEQ ID NO: 627 - DNA, 26 bp, Artificial Sequence
  Description of Artificial Sequence Synthetic primer
gatgaaatgaaaagaggcgatgttcc

SEQ ID NO: 628 - DNA, 22 bp, Artificial Sequence
  Description of Artificial Sequence Synthetic primer
tcaaatgcttgatgtgctcacg

SEQ ID NO: 629 - DNA, 26 bp, Artificial Sequence
  Description of Artificial Sequence Synthetic primer
tggaaggagatgaataatgaaaatgg

SEQ ID NO: 630 - DNA, 28 bp, Artificial Sequence
  Description of Artificial Sequence Synthetic primer
gatcttttgatagagtattttgagaagc

SEQ ID NO: 631 - DNA, 23 bp, Artificial Sequence
  Description of Artificial Sequence Synthetic primer
tcacttagaaccacaaaagaccc

SEQ ID NO: 632 - DNA, 22 bp, Artificial Sequence
  Description of Artificial Sequence Synthetic primer
gccaacaaataagccatgttgc

SEQ ID NO: 633 - DNA, 23 bp, Artificial Sequence
  Description of Artificial Sequence Synthetic primer
acagatttagaggcagagatggg

SEQ ID NO: 634 - DNA, 21 bp, Artificial Sequence
  Description of Artificial Sequence Synthetic primer
gtggtgaaactcttggcttcc

SEQ ID NO: 635 - DNA, 20 bp, Artificial Sequence
  Description of Artificial Sequence Synthetic primer
agcttcacaccttggaatgg

SEQ ID NO: 636 - DNA, 22 bp, Artificial Sequence
  Description of Artificial Sequence Synthetic primer
atccaacttagccaactcaagc

SEQ ID NO: 637 - DNA, 20 bp, Artificial Sequence
  Description of Artificial Sequence Synthetic primer
tgcaagagatcgtgtggtgg

SEQ ID NO: 638 - DNA, 24 bp, Artificial Sequence
  Description of Artificial Sequence Synthetic primer
actgcatttgtgaaaagttcaagc

SEQ ID NO: 639 - DNA, 22 bp, Artificial Sequence
  Description of Artificial Sequence Synthetic primer
tgggacataagggcaataaggg

SEQ ID NO: 640 - DNA, 27 bp, Artificial Sequence
  Description of Artificial Sequence Synthetic primer
cgatcacttcattaccaaaattaagca

SEQ ID NO: 641 - DNA, 20 bp, Artificial Sequence
  Description of Artificial Sequence Synthetic primer
tcagttggtggtcatgcagc

SEQ ID NO: 642 - DNA, 26 bp, Artificial Sequence
  Description of Artificial Sequence Synthetic primer
tgaagttcctaaatcatcactaagcc

SEQ ID NO: 643 - DNA, 20 bp, Artificial Sequence
  Description of Artificial Sequence Synthetic primer
tcagtggagtgctacatggc

SEQ ID NO: 644 - DNA, 21 bp, Artificial Sequence
  Description of Artificial Sequence Synthetic primer
tcaccattatttttggcaggc

SEQ ID NO: 645 - DNA, 30 bp, Artificial Sequence
  Description of Artificial Sequence Synthetic primer
acaaagagagttatgtgaagattattgagc

SEQ ID NO: 646 - DNA, 24 bp, Artificial Sequence
  Description of Artificial Sequence Synthetic primer
tcaagtgtctctaaagggttttcc

SEQ ID NO: 647 - DNA, 23 bp, Artificial Sequence
  Description of Artificial Sequence Synthetic primer
acttcatggtcttcatcctttgg

SEQ ID NO: 648 - DNA, 20 bp, Artificial Sequence
  Description of Artificial Sequence Synthetic primer
ttccctaagcaaccgaaagc

SEQ ID NO: 649 - DNA, 20 bp, Artificial Sequence
  Description of Artificial Sequence Synthetic primer
gccctaatgcaccctattcg

SEQ ID NO: 650 - DNA, 28 bp, Artificial Sequence
  Description of Artificial Sequence Synthetic primer
taattctttttgatgtagttgttgaagc

SEQ ID NO: 651 - DNA, 24 bp, Artificial Sequence
  Description of Artificial Sequence Synthetic primer
tgatgaccaagttgatttctttgc

SEQ ID NO: 652 - DNA, 24 bp, Artificial Sequence
  Description of Artificial Sequence Synthetic primer
tttctaactgcttcagtaaaaggc

SEQ ID NO: 653 - DNA, 29 bp, Artificial Sequence
  Description of Artificial Sequence Synthetic primer
actagagtacatgaaagtatgttacaaga

SEQ ID NO: 654 - DNA, 21 bp, Artificial Sequence
  Description of Artificial Sequence Synthetic primer
acatagctcgcacagtatgga

SEQ ID NO: 655 - DNA, 21 bp, Artificial Sequence
  Description of Artificial Sequence Synthetic primer
aggaagcccaatggttacaca

SEQ ID NO: 656 - DNA, 25 bp, Artificial Sequence
  Description of Artificial Sequence Synthetic primer
agttcgtataatcttaggttggggt

SEQ ID NO: 657 - DNA, 19 bp, Artificial Sequence
  Description of Artificial Sequence Synthetic primer
gtagtggcttccgctgtgg

SEQ ID NO: 658 - DNA, 24 bp, Artificial Sequence
  Description of Artificial Sequence Synthetic primer
tcgccttctctatacatctcatgg

SEQ ID NO: 659 - DNA, 24 bp, Artificial Sequence
  Description of Artificial Sequence Synthetic primer
tggtcttcatcctttggaaaaccc

SEQ ID NO: 660 - DNA, 21 bp, Artificial Sequence
  Description of Artificial Sequence Synthetic primer
ttccctaagcaaccgaaagcg

SEQ ID NO: 661 - DNA, 22 bp, Artificial Sequence
  Description of Artificial Sequence Synthetic primer
gtgaaacatgccctaatgcacc

SEQ ID NO: 662 - DNA, 25 bp, Artificial Sequence
  Description of Artificial Sequence Synthetic primer
atctagtttggcagttaaaagaagc

SEQ ID NO: 663 - DNA, 26 bp, Artificial Sequence
  Description of Artificial Sequence Synthetic primer
acagaatagtggagtgttacatttgg

SEQ ID NO: 664 - DNA, 28 bp, Artificial Sequence
  Description of Artificial Sequence Synthetic primer
ataatagtaagcaaagaaatcaacttgg

SEQ ID NO: 665 - DNA, 23 bp, Artificial Sequence
  Description of Artificial Sequence Synthetic primer
ttcaaaactcccaaaaagcgaaa

SEQ ID NO: 666 - DNA, 21 bp, Artificial Sequence
  Description of Artificial Sequence Synthetic primer
tgtcacgtcagcatacactcc

SEQ ID NO: 667 - DNA, 23 bp, Artificial Sequence
  Description of Artificial Sequence Synthetic primer
tcaaaagatgtagtggcttctgc

SEQ ID NO: 668 - DNA, 24 bp, Artificial Sequence
  Description of Artificial Sequence Synthetic primer
attaagagtggtctaggaatagcg

SEQ ID NO: 669 - DNA, 26 bp, Artificial Sequence
  Description of Artificial Sequence Synthetic primer
caatatggtgtaacagatgaagaagc

SEQ ID NO: 670 - DNA, 26 bp, Artificial Sequence
  Description of Artificial Sequence Synthetic primer
tgagagtatcaatcaaattcttgagc

SEQ ID NO: 671 - DNA, 23 bp, Artificial Sequence
  Description of Artificial Sequence Synthetic primer
catcgagacaaactgcgaatact

SEQ ID NO: 672 - DNA, 27 bp, Artificial Sequence
  Description of Artificial Sequence Synthetic primer
atatgtgatctccattttcttccattg

SEQ ID NO: 673 - DNA, 19 bp, Artificial Sequence
  Description of Artificial Sequence Synthetic primer
catggatggcccgattgga

SEQ ID NO: 674 - DNA, 25 bp, Artificial Sequence
  Description of Artificial Sequence Synthetic primer
tcgaaaatgattctttccaagccat

SEQ ID NO: 675 - DNA, 23 bp, Artificial Sequence
  Description of Artificial Sequence Synthetic primer
gattggggacttagtgaaatggg

SEQ ID NO: 676 - DNA, 28 bp, Artificial Sequence
  Description of Artificial Sequence Synthetic primer
gtaaagttttctaactcggtgattaagg

SEQ ID NO: 677 - DNA, 21 bp, Artificial Sequence
  Description of Artificial Sequence Synthetic primer
ggtgggatggtgaaggattga

SEQ ID NO: 678 - DNA, 24 bp, Artificial Sequence
  Description of Artificial Sequence Synthetic primer
tgatgtcacttgtttcttgttgat

SEQ ID NO: 679 - DNA, 21 bp, Artificial Sequence
  Description of Artificial Sequence Synthetic primer
tcacaccttgcttcttccagc

SEQ ID NO: 680 - DNA, 30 bp, Artificial Sequence
  Description of Artificial Sequence Synthetic primer
tcattcaacaaacgacaaataatcataacc

SEQ ID NO: 681 - DNA, 21 bp, Artificial Sequence
  Description of Artificial Sequence Synthetic primer
acttgaagaacaatcccgagg

SEQ ID NO: 682 - DNA, 22 bp, Artificial Sequence
  Description of Artificial Sequence Synthetic primer
aaccctaacatcatcatcgtcg

SEQ ID NO: 683 - DNA, 21 bp, Artificial Sequence
  Description of Artificial Sequence Synthetic primer
cagacactgaaatgcttgacg

SEQ ID NO: 684 - DNA, 22 bp, Artificial Sequence
  Description of Artificial Sequence Synthetic primer
tagcggttagacatttagaggg

SEQ ID NO: 685 - DNA, 23 bp, Artificial Sequence
  Description of Artificial Sequence Synthetic primer
caaccctctaaatgtctaaccgc

SEQ ID NO: 686 - DNA, 26 bp, Artificial Sequence
  Description of Artificial Sequence Synthetic primer
aaattgagttttgaaggcatagtagg

SEQ ID NO: 687 - DNA, 20 bp, Artificial Sequence
  Description of Artificial Sequence Synthetic primer
atctttgccccaaactcagg

SEQ ID NO: 688 - DNA, 22 bp, Artificial Sequence
  Description of Artificial Sequence Synthetic primer
ggttaggatgtggtatcatggc

SEQ ID NO: 689 - DNA, 23 bp, Artificial Sequence
  Description of Artificial Sequence Synthetic primer
taccacatcctaaccaaccttcg

SEQ ID NO: 690 - DNA, 21 bp, Artificial Sequence
  Description of Artificial Sequence Synthetic primer
caacaacacaagccagagtgg

SEQ ID NO: 691 - DNA, 26 bp, Artificial Sequence
  Description of Artificial Sequence Synthetic primer
tattcattcatctaatgcaaaaaggc

SEQ ID NO: 692 - DNA, 22 bp, Artificial Sequence
  Description of Artificial Sequence Synthetic primer
aatatgtccgaaacgaaaacgg

SEQ ID NO: 693 - DNA, 25 bp, Artificial Sequence
  Description of Artificial Sequence Synthetic primer
tgttatcatatcttgaagtgttgcc

SEQ ID NO: 694 - DNA, 20 bp, Artificial Sequence
  Description of Artificial Sequence Synthetic primer
ctgttgccgatggagattgg

SEQ ID NO: 695 - DNA, 20 bp, Artificial Sequence
  Description of Artificial Sequence Synthetic primer
caatctccatcggcaacagc

SEQ ID NO: 696 - DNA, 29 bp, Artificial Sequence
  Description of Artificial Sequence Synthetic primer
tgttattattggaaaacttgtgaactagg

SEQ ID NO: 697 - DNA, 21 bp, Artificial Sequence
  Description of Artificial Sequence Synthetic primer
agggttagctgagcatttcgc

SEQ ID NO: 698 - DNA, 24 bp, Artificial Sequence
  Description of Artificial Sequence Synthetic primer
ctaggaaaaactttgtagccatgc

SEQ ID NO: 699 - DNA, 23 bp, Artificial Sequence
  Description of Artificial Sequence Synthetic primer
actatgaatgcttttcggttacg

SEQ ID NO: 700 - DNA, 28 bp, Artificial Sequence
  Description of Artificial Sequence Synthetic primer
aaaatagacttttgaagtagttttctcg

SEQ ID NO: 701 - DNA, 24 bp, Artificial Sequence
  Description of Artificial Sequence Synthetic primer
ttgcttttcatggtgaatttgagc

SEQ ID NO: 702 - DNA, 27 bp, Artificial Sequence
  Description of Artificial Sequence Synthetic primer
tttgttaaaaggatttgtatgtggagc

SEQ ID NO: 703 - DNA, 21 bp, Artificial Sequence
  Description of Artificial Sequence Synthetic primer
tggctcgactagatcacttgg

SEQ ID NO: 704 - DNA, 22 bp, Artificial Sequence
  Description of Artificial Sequence Synthetic primer
gtgaaacctcgacaacctttgg

SEQ ID NO: 705 - DNA, 25 bp, Artificial Sequence
  Description of Artificial Sequence Synthetic primer
ggggacttaatgaaatgggatttgg

SEQ ID NO: 706 - DNA, 23 bp, Artificial Sequence
  Description of Artificial Sequence Synthetic primer
tcataaggcaaagaacaacaagc

SEQ ID NO: 707 - DNA, 21 bp, Artificial Sequence
  Description of Artificial Sequence Synthetic primer
ccatagcaactcacaccttgc

SEQ ID NO: 708 - DNA, 23 bp, Artificial Sequence
  Description of Artificial Sequence Synthetic primer
acactctcacattgaattggtcg

SEQ ID NO: 709 - DNA, 25 bp, Artificial Sequence
  Description of Artificial Sequence Synthetic primer
cttgcttttcatggtgaatttgagc

SEQ ID NO: 710 - DNA, 30 bp, Artificial Sequence
  Description of Artificial Sequence Synthetic primer
cagtttcttaaagggatttgtatgttgagc

SEQ ID NO: 711 - DNA, 20 bp, Artificial Sequence
  Description of Artificial Sequence Synthetic primer
gatggctcgactagatcacc

SEQ ID NO: 712 - DNA, 24 bp, Artificial Sequence
  Description of Artificial Sequence Synthetic primer
agatgtctttcccatccataaagc

SEQ ID NO: 713 - DNA, 24 bp, Artificial Sequence
  Description of Artificial Sequence Synthetic primer
agaatggggacttagtgaaatggg

SEQ ID NO: 714 - DNA, 26 bp, Artificial Sequence
  Description of Artificial Sequence Synthetic primer
tggagaatcataaggcaaataacagc

SEQ ID NO: 715 - DNA, 24 bp, Artificial Sequence
  Description of Artificial Sequence Synthetic primer
tggaataatgaagggttgagtggt

SEQ ID NO: 716 - DNA, 25 bp, Artificial Sequence
  Description of Artificial Sequence Synthetic primer
tcttatgtagctggttatgtcttca

SEQ ID NO: 717 - DNA, 24 bp, Artificial Sequence
  Description of Artificial Sequence Synthetic primer
tgaatctgagtggagtaaaagtgg

SEQ ID NO: 718 - DNA, 24 bp, Artificial Sequence
  Description of Artificial Sequence Synthetic primer
agaagaaatgaagctggaagaagc

SEQ ID NO: 719 - DNA, 23 bp, Artificial Sequence
  Description of Artificial Sequence Synthetic primer
gcaactcataccttgcttcttcc

SEQ ID NO: 720 - DNA, 26 bp, Artificial Sequence
  Description of Artificial Sequence Synthetic primer
gaactttgtaagtcattcaacaaacg

SEQ ID NO: 721 - DNA, 24 bp, Artificial Sequence
  Description of Artificial Sequence Synthetic primer
gagggaaagagaagaaggcaaacc

SEQ ID NO: 722 - DNA, 28 bp, Artificial Sequence
  Description of Artificial Sequence Synthetic primer
acttttaagcaagataagtgtagaagcc

SEQ ID NO: 723 - DNA, 26 bp, Artificial Sequence
  Description of Artificial Sequence Synthetic primer
aatgacaatggttttaccaatcttcc

SEQ ID NO: 724 - DNA, 25 bp, Artificial Sequence
  Description of Artificial Sequence Synthetic primer
aatcgctttgttaatgtcttcaagc

SEQ ID NO: 725 - DNA, 21 bp, Artificial Sequence
  Description of Artificial Sequence Synthetic primer
tcggccaattttcatcctagt

SEQ ID NO: 726 - DNA, 26 bp, Artificial Sequence
  Description of Artificial Sequence Synthetic primer
ggtatatttgagaaagtgatctcccc

SEQ ID NO: 727 - DNA, 19 bp, Artificial Sequence
  Description of Artificial Sequence Synthetic primer
ggatgctaactgctgcccc

SEQ ID NO: 728 - DNA, 21 bp, Artificial Sequence
  Description of Artificial Sequence Synthetic primer
aaagggagaccgtatgaaggg

SEQ ID NO: 729 - DNA, 22 bp, Artificial Sequence
  Description of Artificial Sequence Synthetic primer
gaaatcccagttgaccatgagc

SEQ ID NO: 730 - DNA, 20 bp, Artificial Sequence
  Description of Artificial Sequence Synthetic primer
gatgactgtggttagggtcg

SEQ ID NO: 731 - DNA, 21 bp, Artificial Sequence
  Description of Artificial Sequence Synthetic primer
cacaactcatttggtggaggc

SEQ ID NO: 732 - DNA, 21 bp, Artificial Sequence
  Description of Artificial Sequence Synthetic primer
agatgactgtggttagggtcg

SEQ ID NO: 733 - DNA, 23 bp, Artificial Sequence
  Description of Artificial Sequence Synthetic primer
ggaaagacttggactttgcatcg

SEQ ID NO: 734 - DNA, 21 bp, Artificial Sequence
  Description of Artificial Sequence Synthetic primer
ctagcagcactgtactttggc

SEQ ID NO: 735 - DNA, 23 bp, Artificial Sequence
  Description of Artificial Sequence Synthetic primer
ggaaagacttggactttgcatcg

SEQ ID NO: 736 - DNA, 20 bp, Artificial Sequence
  Description of Artificial Sequence Synthetic primer
ttgcgtctgtgaagagttcc

SEQ ID NO: 737 - DNA, 28 bp, Artificial Sequence
  Description of Artificial Sequence Synthetic primer
tatacgatgaaattgaggaggagttggc

SEQ ID NO: 738 - DNA, 22 bp, Artificial Sequence
  Description of Artificial Sequence Synthetic primer
aggccatccgataagttctgcc

SEQ ID NO: 739 - DNA, 21 bp, Artificial Sequence
  Description of Artificial Sequence Synthetic primer
tactacgtggaagctcaatgg

SEQ ID NO: 740 - DNA, 21 bp, Artificial Sequence
  Description of Artificial Sequence Synthetic primer
aagccaatgaaagacctcagc

SEQ ID NO: 741 - DNA, 20 bp, Artificial Sequence
  Description of Artificial Sequence Synthetic primer
actacatgctcactgccacg

SEQ ID NO: 742 - DNA, 21 bp, Artificial Sequence
  Description of Artificial Sequence Synthetic primer
tctgcaaactactgctgatgc

SEQ ID NO: 743 - DNA, 23 bp, Artificial Sequence
  Description of Artificial Sequence Synthetic primer
gtttgagcaagatagaggacacg

SEQ ID NO: 744 - DNA, 20 bp, Artificial Sequence
  Description of Artificial Sequence Synthetic primer
gcatgaggacagacatgggc

SEQ ID NO: 745 - DNA, 21 bp, Artificial Sequence
  Description of Artificial Sequence Synthetic primer
tgaatgaggagtgcatggagc

SEQ ID NO: 746 - DNA, 19 bp, Artificial Sequence
  Description of Artificial Sequence Synthetic primer
tccggcatgggtgtaaccg

SEQ ID NO: 747 - DNA, 24 bp, Artificial Sequence
  Description of Artificial Sequence Synthetic primer
agcctcatcacaaaatgacaaagt

SEQ ID NO: 748 - DNA, 24 bp, Artificial Sequence
  Description of Artificial Sequence Synthetic primer
ggttgataagttgttgttggacga

SEQ ID NO: 749 - DNA, 22 bp, Artificial Sequence
  Description of Artificial Sequence Synthetic primer
aaggaagtggtaaggagagagg

SEQ ID NO: 750 - DNA, 23 bp, Artificial Sequence
  Description of Artificial Sequence Synthetic primer
gaaaccataaatccatgttggcg

SEQ ID NO: 751 - DNA, 23 bp, Artificial Sequence
  Description of Artificial Sequence Synthetic primer
agcaagggaattttaaggagtgc

SEQ ID NO: 752 - DNA, 20 bp, Artificial Sequence
  Description of Artificial Sequence Synthetic primer
aggtgagtggtggtgaaagc

SEQ ID NO: 753 - DNA, 21 bp, Artificial Sequence
  Description of Artificial Sequence Synthetic primer
ttgctttcaccaccactcacc

SEQ ID NO: 754 - DNA, 21 bp, Artificial Sequence
  Description of Artificial Sequence Synthetic primer
gaaatgtaatgcctggcgtgg

SEQ ID NO: 755 - DNA, 23 bp, Artificial Sequence
  Description of Artificial Sequence Synthetic primer
tgcaagagataggattgtggagc

SEQ ID NO: 756 - DNA, 20 bp, Artificial Sequence
  Description of Artificial Sequence Synthetic primer
tctgcaactgaggccaatgc

SEQ ID NO: 757 - DNA, 21 bp, Artificial Sequence
  Description of Artificial Sequence Synthetic primer
tgaacctgaattgtcactggc

SEQ ID NO: 758 - DNA, 22 bp, Artificial Sequence
  Description of Artificial Sequence Synthetic primer
gctcaagctcttcaaatgtacc

SEQ ID NO: 759 - DNA, 20 bp, Artificial Sequence
  Description of Artificial Sequence Synthetic primer
aattgtgcggatcaacttcg

SEQ ID NO: 760 - DNA, 23 bp, Artificial Sequence
  Description of Artificial Sequence Synthetic primer
tgtaactttcctcctttccaagc

SEQ ID NO: 761 - DNA, 24 bp, Artificial Sequence
  Description of Artificial Sequence Synthetic primer
agcgatgaaaagattacttggagc

SEQ ID NO: 762 - DNA, 27 bp, Artificial Sequence
  Description of Artificial Sequence Synthetic primer
catccatgaacctacaaaggatattgc

SEQ ID NO: 763 - DNA, 22 bp, Artificial Sequence
  Description of Artificial Sequence Synthetic primer
agagatcattcaccgtctaccg

SEQ ID NO: 764 - DNA, 26 bp, Artificial Sequence
  Description of Artificial Sequence Synthetic primer
aaactggataaggcacattagaaggc

SEQ ID NO: 765 - DNA, 22 bp, Artificial Sequence
  Description of Artificial Sequence Synthetic primer
ggccttctaatgtgccttatcc

SEQ ID NO: 766 - DNA, 27 bp, Artificial Sequence
  Description of Artificial Sequence Synthetic primer
tcatgggatttgatctataagtaacgc

SEQ ID NO: 767 - DNA, 26 bp, Artificial Sequence
  Description of Artificial Sequence Synthetic primer
agttttagcctcatcacaaaatgaca

SEQ ID NO: 768 - DNA, 24 bp, Artificial Sequence
  Description of Artificial Sequence Synthetic primer
gttgataagttgttgttggacgaa

SEQ ID NO: 769 - DNA, 24 bp, Artificial Sequence
  Description of Artificial Sequence Synthetic primer
tagcctcatcacaaaatgacaaag

SEQ ID NO: 770 - DNA, 24 bp, Artificial Sequence
  Description of Artificial Sequence Synthetic primer
cccccaaatagaaggttgataagt

SEQ ID NO: 771 - DNA, 20 bp, Artificial Sequence
  Description of Artificial Sequence Synthetic primer
taaaaagcagcgagttgacg

SEQ ID NO: 772 - DNA, 22 bp, Artificial Sequence
  Description of Artificial Sequence Synthetic primer
aatgatacgacaatcccaaacg

SEQ ID NO: 773 - DNA, 22 bp, Artificial Sequence
  Description of Artificial Sequence Synthetic primer
aaggaagtggtaaggagagagg

SEQ ID NO: 774 - DNA, 23 bp, Artificial Sequence
  Description of Artificial Sequence Synthetic primer
gaaaccataaatccatgttggcg

SEQ ID NO: 775 - DNA, 23 bp, Artificial Sequence
  Description of Artificial Sequence Synthetic primer
agcaagggaattttaaggagtgc

SEQ ID NO: 776 - DNA, 20 bp, Artificial Sequence
  Description of Artificial Sequence Synthetic primer
aggtgagtggtggtgaaagc

SEQ ID NO: 777 - DNA, 22 bp, Artificial Sequence
  Description of Artificial Sequence Synthetic primer
cttgctttcaccaccactcacc

SEQ ID NO: 778 - DNA, 19 bp, Artificial Sequence
  Description of Artificial Sequence Synthetic primer
gtaatgcctggcgtggagc

SEQ ID NO: 779 - DNA, 22 bp, Artificial Sequence
  Description of Artificial Sequence Synthetic primer
gcaagagataggattgtggagc

SEQ ID NO: 780 - DNA, 20 bp, Artificial Sequence
  Description of Artificial Sequence Synthetic primer
atctgcaactgaggccaagg

SEQ ID NO: 781 - DNA, 21 bp, Artificial Sequence
  Description of Artificial Sequence Synthetic primer
tgaacctgaattgtcactggc

SEQ ID NO: 782 - DNA, 22 bp, Artificial Sequence
  Description of Artificial Sequence Synthetic primer
gctcaagctcttcaaatgtacc

SEQ ID NO: 783 - DNA, 20 bp, Artificial Sequence
  Description of Artificial Sequence Synthetic primer
aattgtgcggatcaacttcg

SEQ ID NO: 784 - DNA, 23 bp, Artificial Sequence
  Description of Artificial Sequence Synthetic primer
tgtaactttcctcctttccaagc

SEQ ID NO: 785 - DNA, 20 bp, Artificial Sequence
  Description of Artificial Sequence Synthetic primer
cagtgaagctcgatggttgc

SEQ ID NO: 786 - DNA, 25 bp, Artificial Sequence
  Description of Artificial Sequence Synthetic primer
tccatgaacctacaaaggatattgc

SEQ ID NO: 787 - DNA, 21 bp, Artificial Sequence
  Description of Artificial Sequence Synthetic primer
ccgttgagagttacatgaggc

SEQ ID NO: 788 - DNA, 23 bp, Artificial Sequence
  Description of Artificial Sequence Synthetic primer
actggataaggcacattagaagg

SEQ ID NO: 789 - DNA, 20 bp, Artificial Sequence
  Description of Artificial Sequence Synthetic primer
acaagaggcatgtgatgagc

SEQ ID NO: 790 - DNA, 26 bp, Artificial Sequence
  Description of Artificial Sequence Synthetic primer
gggatttgatctataagtaacgcagc

SEQ ID NO: 791 - DNA, 23 bp, Artificial Sequence
  Description of Artificial Sequence Synthetic primer
tgtcgtctcaaatcttagcaacc

SEQ ID NO: 792 - DNA, 20 bp, Artificial Sequence
  Description of Artificial Sequence Synthetic primer
atgcaaaaatcggtctcccc

SEQ ID NO: 793 - DNA, 26 bp, Artificial Sequence
  Description of Artificial Sequence Synthetic primer
ttcttatgtgatgattggagtaatcg

SEQ ID NO: 794 - DNA, 28 bp, Artificial Sequence
  Description of Artificial Sequence Synthetic primer
gcttattttgttgtaaatgtgttgaagc

SEQ ID NO: 795 - DNA, 25 bp, Artificial Sequence
  Description of Artificial Sequence Synthetic primer
cgattgaagttaattgatgtggtgc

SEQ ID NO: 796 - DNA, 26 bp, Artificial Sequence
  Description of Artificial Sequence Synthetic primer
aaagaaaccctatatccatgttgtcg

SEQ ID NO: 797 - DNA, 21 bp, Artificial Sequence
  Description of Artificial Sequence Synthetic primer
gaatgtttggcgagtgacacc

SEQ ID NO: 798 - DNA, 20 bp, Artificial Sequence
  Description of Artificial Sequence Synthetic primer
aagggcctctctagggctcg

SEQ ID NO: 799 - DNA, 22 bp, Artificial Sequence
  Description of Artificial Sequence Synthetic primer
aagaacaccccaatgatgatcc

SEQ ID NO: 800 - DNA, 23 bp, Artificial Sequence
  Description of Artificial Sequence Synthetic primer
actaagctcctttttgtgcatgg

SEQ ID NO: 801 - DNA, 22 bp, Artificial Sequence
  Description of Artificial Sequence Synthetic primer
ggtggaaggaattagacagtgc

SEQ ID NO: 802 - DNA, 20 bp, Artificial Sequence
  Description of Artificial Sequence Synthetic primer
tcagcgattgaggaaagtgc

SEQ ID NO: 803 - DNA, 27 bp, Artificial Sequence
  Description of Artificial Sequence Synthetic primer
gggacaaaaattgtatggataaactcc

SEQ ID NO: 804 - DNA, 27 bp, Artificial Sequence
  Description of Artificial Sequence Synthetic primer
tccttttcaaactcttgttcaaattcc

SEQ ID NO: 805 - DNA, 21 bp, Artificial Sequence
  Description of Artificial Sequence Synthetic primer
agctcgatggttgaatgaagg

SEQ ID NO: 806 - DNA, 20 bp, Artificial Sequence
  Description of Artificial Sequence Synthetic primer
ggtctttggagagccactcg

SEQ ID NO: 807 - DNA, 22 bp, Artificial Sequence
  Description of Artificial Sequence Synthetic primer
tgatggcttgctctttagttgg

SEQ ID NO: 808 - DNA, 20 bp, Artificial Sequence
  Description of Artificial Sequence Synthetic primer
agccacgtcatccatgtacc

SEQ ID NO: 809 - DNA, 28 bp, Artificial Sequence
  Description of Artificial Sequence Synthetic primer
agaatgagcaagagagaaatcatatacc

SEQ ID NO: 810 - DNA, 20 bp, Artificial Sequence
  Description of Artificial Sequence Synthetic primer
ccatgcaataaccactcgcc

SEQ ID NO: 811 - DNA, 21 bp, Artificial Sequence
  Description of Artificial Sequence Synthetic primer
tctcaaaccaactgaagcagc

SEQ ID NO: 812 - DNA, 22 bp, Artificial Sequence
  Description of Artificial Sequence Synthetic primer
ttggatcaatgagcaagacagc

SEQ ID NO: 813 - DNA, 23 bp, Artificial Sequence
  Description of Artificial Sequence Synthetic primer
ggtcttcatcctttggaaaatcc

SEQ ID NO: 814 - DNA, 21 bp, Artificial Sequence
  Description of Artificial Sequence Synthetic primer
aagcaaccgaaatcgaagagc

SEQ ID NO: 815 - DNA, 22 bp, Artificial Sequence
  Description of Artificial Sequence Synthetic primer
agcttcacaaatgagagttcgc

SEQ ID NO: 816 - DNA, 21 bp, Artificial Sequence
  Description of Artificial Sequence Synthetic primer
tcgtgagaaggtagttgatgg

SEQ ID NO: 817 - DNA, 26 bp, Artificial Sequence
  Description of Artificial Sequence Synthetic primer
acagaatagtggagtgttacatttgg

SEQ ID NO: 818 - DNA, 28 bp, Artificial Sequence
  Description of Artificial Sequence Synthetic primer
ataatagtaagcaaagaaatcaacttgg

SEQ ID NO: 819 - DNA, 24 bp, Artificial Sequence
  Description of Artificial Sequence Synthetic primer
acaacttgccagagtacatgaaag

SEQ ID NO: 820 - DNA, 23 bp, Artificial Sequence
  Description of Artificial Sequence Synthetic primer
cgcgcagtatgaattttcctttg

SEQ ID NO: 821 - DNA, 26 bp, Artificial Sequence
  Description of Artificial Sequence Synthetic primer
ggatctgaagagctaatatcaatggc

SEQ ID NO: 822 - DNA, 24 bp, Artificial Sequence
  Description of Artificial Sequence Synthetic primer
agttcgtataatcttaggttgggg

SEQ ID NO: 823 - DNA, 23 bp, Artificial Sequence
  Description of Artificial Sequence Synthetic primer
tcaaaagatgtagtggcttctgc

SEQ ID NO: 824 - DNA, 24 bp, Artificial Sequence
  Description of Artificial Sequence Synthetic primer
attaagagtggtctaggaatagcg

SEQ ID NO: 825 - DNA, 26 bp, Artificial Sequence
  Description of Artificial Sequence Synthetic primer
caatatggtgtaacagatgaagaagc

SEQ ID NO: 826 - DNA, 26 bp, Artificial Sequence
  Description of Artificial Sequence Synthetic primer
tgagagtatcaatcaaattcttgagc

SEQ ID NO: 827 - DNA, 20 bp, Artificial Sequence
  Description of Artificial Sequence Synthetic primer
agccaactttgaaccatcca

SEQ ID NO: 828 - DNA, 28 bp, Artificial Sequence
  Description of Artificial Sequence Synthetic primer
aaagagactgaatgaaatcaaaagacca

SEQ ID NO: 829 - DNA, 22 bp, Artificial Sequence
  Description of Artificial Sequence Synthetic primer
gaggaagatgtgaaaaggatgc

SEQ ID NO: 830 - DNA, 22 bp, Artificial Sequence
  Description of Artificial Sequence Synthetic primer
gcctaaattcaagagcagtggc

SEQ ID NO: 831 - DNA, 28 bp, Artificial Sequence
  Description of Artificial Sequence Synthetic primer
caggaaaattcaaaacaaacataagtgg

SEQ ID NO: 832 - DNA, 23 bp, Artificial Sequence
  Description of Artificial Sequence Synthetic primer
agcttcctctaaaatgctttcgc

SEQ ID NO: 833 - DNA, 24 bp, Artificial Sequence
  Description of Artificial Sequence Synthetic primer
aggcgaaagcattttagaggaagc

SEQ ID NO: 834 - DNA, 24 bp, Artificial Sequence
  Description of Artificial Sequence Synthetic primer
agcttctattcttgtggtccttcg

SEQ ID NO: 835 - DNA, 22 bp, Artificial Sequence
  Description of Artificial Sequence Synthetic primer
ggtggaggcatactaaacttgg

SEQ ID NO: 836 - DNA, 28 bp, Artificial Sequence
  Description of Artificial Sequence Synthetic primer
tgaaaagctctaattcatctaatgttcc

SEQ ID NO: 837 - DNA, 25 bp, Artificial Sequence
  Description of Artificial Sequence Synthetic primer
gatgggatgtggaaatgataaatga

SEQ ID NO: 838 - DNA, 24 bp, Artificial Sequence
  Description of Artificial Sequence Synthetic primer
attttgatggtgatgtgttgatct

SEQ ID NO: 839 - DNA, 29 bp, Artificial Sequence
  Description of Artificial Sequence Synthetic primer
ctacaagaagcaaaatggtattacagtgg

SEQ ID NO: 840 - DNA, 23 bp, Artificial Sequence
  Description of Artificial Sequence Synthetic primer
gcacaataagaactggtgctccc

SEQ ID NO: 841 - DNA, 22 bp, Artificial Sequence
  Description of Artificial Sequence Synthetic primer
ggagcaccagttcttattgtgc

SEQ ID NO: 842 - DNA, 23 bp, Artificial Sequence
  Description of Artificial Sequence Synthetic primer
gcactgtgacgaattatggtagg

SEQ ID NO: 843 - DNA, 20 bp, Artificial Sequence
  Description of Artificial Sequence Synthetic primer
aaagaggtgatgctccgacg

SEQ ID NO: 844 - DNA, 23 bp, Artificial Sequence
  Description of Artificial Sequence Synthetic primer
agattatcctgagaactgtgacc

SEQ ID NO: 845 - DNA, 26 bp, Artificial Sequence
  Description of Artificial Sequence Synthetic primer
tcagaagagatcagcaaactatcaac

SEQ ID NO: 846 - DNA, 25 bp, Artificial Sequence
  Description of Artificial Sequence Synthetic primer
cttgaaaggagtagaaagtgattga

SEQ ID NO: 847 - DNA, 24 bp, Artificial Sequence
  Description of Artificial Sequence Synthetic primer
tgcaaagacttggaatctcttacc

SEQ ID NO: 848 - DNA, 24 bp, Artificial Sequence
  Description of Artificial Sequence Synthetic primer
catacacatttttgttggtgttgc

SEQ ID NO: 849 - DNA, 23 bp, Artificial Sequence
  Description of Artificial Sequence Synthetic primer
gaggcaattttatggtgtcttcc

SEQ ID NO: 850 - DNA, 23 bp, Artificial Sequence
  Description of Artificial Sequence Synthetic primer
ttgttttgtgtcttgtctcttcc

SEQ ID NO: 851 - DNA, 25 bp, Artificial Sequence
  Description of Artificial Sequence Synthetic primer
ggacaataggagtttcatttgagcc

SEQ ID NO: 852 - DNA, 23 bp, Artificial Sequence
  Description of Artificial Sequence Synthetic primer
tctcaaccacattagtgaagagc

SEQ ID NO: 853 - DNA, 24 bp, Artificial Sequence
  Description of Artificial Sequence Synthetic primer
tgggatgttagtgctatgaatggg

SEQ ID NO: 854 - DNA, 30 bp, Artificial Sequence
  Description of Artificial Sequence Synthetic primer
gaggctttttccttttaacacatcaaatgc

SEQ ID NO: 855 - DNA, 28 bp, Artificial Sequence
  Description of Artificial Sequence Synthetic primer
taagagaagcaagatggtattatgatgg

SEQ ID NO: 856 - DNA, 27 bp, Artificial Sequence
  Description of Artificial Sequence Synthetic primer
gtattatggtagggtatccattaaagc

SEQ ID NO: 857 - DNA, 23 bp, Artificial Sequence
  Description of Artificial Sequence Synthetic primer
cgtgtatgtgaagagaaagctcg

SEQ ID NO: 858 - DNA, 23 bp, Artificial Sequence
  Description of Artificial Sequence Synthetic primer
agtacatgatcttttgtttggcg

SEQ ID NO: 859 - DNA, 21 bp, Artificial Sequence
  Description of Artificial Sequence Synthetic primer
cagccaactttgaaccatcca

SEQ ID NO: 860 - DNA, 22 bp, Artificial Sequence
  Description of Artificial Sequence Synthetic primer
gcttgaaagagactgaacgaaa

SEQ ID NO: 861 - DNA, 23 bp, Artificial Sequence
  Description of Artificial Sequence Synthetic primer
cgacgaagatgtgaaaaggatgc

SEQ ID NO: 862 - DNA, 20 bp, Artificial Sequence
  Description of Artificial Sequence Synthetic primer
accatgttgacgtagaagcc

SEQ ID NO: 863 - DNA, 24 bp, Artificial Sequence
  Description of Artificial Sequence Synthetic primer
agctagaattttcacaactgaacg

SEQ ID NO: 864 - DNA, 23 bp, Artificial Sequence
  Description of Artificial Sequence Synthetic primer
gcttctattcttgtggtccttcg

SEQ ID NO: 865 - DNA, 26 bp, Artificial Sequence
  Description of Artificial Sequence Synthetic primer
tacgatattgaagtagtgaatcatgc

SEQ ID NO: 866 - DNA, 23 bp, Artificial Sequence
  Description of Artificial Sequence Synthetic primer
ttggcaaactcaagcaaaatagg

SEQ ID NO: 867 - DNA, 23 bp, Artificial Sequence
  Description of Artificial Sequence Synthetic primer
tggtggaggcatactaaacttgg

SEQ ID NO: 868 - DNA, 29 bp, Artificial Sequence
  Description of Artificial Sequence Synthetic primer
gtgaaaagctctaattcatctaatgttcc

SEQ ID NO: 869 - DNA, 26 bp, Artificial Sequence
  Description of Artificial Sequence Synthetic primer
gatgggatgtggaaatgataaatgaa

SEQ ID NO: 870 - DNA, 26 bp, Artificial Sequence
  Description of Artificial Sequence Synthetic primer
attttgatggtgatttgttgatctct

SEQ ID NO: 871 - DNA, 29 bp, Artificial Sequence
  Description of Artificial Sequence Synthetic primer
ctacaagaagcaaaatggtactacagtgg

SEQ ID NO: 872 - DNA, 23 bp, Artificial Sequence
  Description of Artificial Sequence Synthetic primer
gcacaataagaactggtgctccc

SEQ ID NO: 873 - DNA, 22 bp, Artificial Sequence
  Description of Artificial Sequence Synthetic primer
ggagcaccagttcttattgtgc

SEQ ID NO: 874 - DNA, 23 bp, Artificial Sequence
  Description of Artificial Sequence Synthetic primer
gcactgtgacgaattatggtagg

SEQ ID NO: 875 - DNA, 19 bp, Artificial Sequence
  Description of Artificial Sequence Synthetic primer
aagaggtgatgctccgacg

SEQ ID NO: 876 - DNA, 24 bp, Artificial Sequence
  Description of Artificial Sequence Synthetic primer
tagattatcctgagaactgtgacc

SEQ ID NO: 877 - DNA, 30 bp, Artificial Sequence; identical to SEQ ID NO: 645
  Description of Artificial Sequence Synthetic primer

SEQ ID NO: 878 - DNA, 21 bp, Artificial Sequence
  Description of Artificial Sequence Synthetic primer
ccgaaagcgaagagcatcagc

SEQ ID NO: 879 - DNA, 27 bp, Artificial Sequence
  Description of Artificial Sequence Synthetic primer
aattacttcatggtcttcaacctttgg

SEQ ID NO: 880 - DNA, 26 bp, Artificial Sequence
  Description of Artificial Sequence Synthetic primer
ctataaaatagccttgttccctaagc

SEQ ID NO: 881 - DNA, 22 bp, Artificial Sequence
  Description of Artificial Sequence Synthetic primer
agcttcacaaatgagagttcgc

SEQ ID NO: 882 - DNA, 20 bp, Artificial Sequence
  Description of Artificial Sequence Synthetic primer
cggatagggtgcattagggc

SEQ ID NO: 883 - DNA, 21 bp, Artificial Sequence
  Description of Artificial Sequence Synthetic primer
atgcaccctatccgaaagagc

SEQ ID NO: 884 - DNA, 25 bp, Artificial Sequence
  Description of Artificial Sequence Synthetic primer
ttccttttgatgtagttgttgaagc

SEQ ID NO: 885 - DNA, 26 bp, Artificial Sequence
  Description of Artificial Sequence Synthetic primer
acagaatagtggagtgttacatttgg

SEQ ID NO: 886 - DNA, 28 bp, Artificial Sequence
  Description of Artificial Sequence Synthetic primer
ataatagtaagcaaagaaatcaacttgg

SEQ ID NO: 887 - DNA, 23 bp, Artificial Sequence
  Description of Artificial Sequence Synthetic primer
tcatcaggtgggatatttttgct

SEQ ID NO: 888 - DNA, 21 bp, Artificial Sequence
  Description of Artificial Sequence Synthetic primer
acataactcgcgcagaatgaa

SEQ ID NO: 889 - DNA, 26 bp, Artificial Sequence
  Description of Artificial Sequence Synthetic primer
ggatctgaagagctaatatcaatggc

SEQ ID NO: 890 - DNA, 22 bp, Artificial Sequence
  Description of Artificial Sequence Synthetic primer
tcgtataatgttaggttggggc

SEQ ID NO: 891 - DNA, 23 bp, Artificial Sequence
  Description of Artificial Sequence Synthetic primer
tcaaaagatgtagtggcttctgc

SEQ ID NO: 892 - DNA, 24 bp, Artificial Sequence
  Description of Artificial Sequence Synthetic primer
attaagagtggtctaggaatagcg

SEQ ID NO: 893 - DNA, 26 bp, Artificial Sequence
  Description of Artificial Sequence Synthetic primer
acaaaaggttgaggaattaaaagagg

SEQ ID NO: 894 - DNA, 20 bp, Artificial Sequence
  Description of Artificial Sequence Synthetic primer
cccacaaggaaacatcatgc

SEQ ID NO: 895 - DNA, 23 bp, Artificial Sequence
  Description of Artificial Sequence Synthetic primer
cacacctaaatgagtttttggcg

SEQ ID NO: 896 - DNA, 22 bp, Artificial Sequence
  Description of Artificial Sequence Synthetic primer
cctgctgatttcactaagctcc

SEQ ID NO: 897 - DNA, 28 bp, Artificial Sequence
  Description of Artificial Sequence Synthetic primer
tgtggatgttaggagtctattatgaacc

SEQ ID NO: 898 - DNA, 24 bp, Artificial Sequence
  Description of Artificial Sequence Synthetic primer
atttcttgccaaagagtattgggg

SEQ ID NO: 899 - DNA, 25 bp, Artificial Sequence
  Description of Artificial Sequence Synthetic primer
ttgttatgaagagtttgagcaagtg

SEQ ID NO: 900 - DNA, 28 bp, Artificial Sequence
  Description of Artificial Sequence Synthetic primer
ctctataagtttcttcttttgtaagcac

SEQ ID NO: 901 - DNA, 23 bp, Artificial Sequence
  Description of Artificial Sequence Synthetic primer
gaagctcgatggttgaatagtgg

SEQ ID NO: 902 - DNA, 27 bp, Artificial Sequence
  Description of Artificial Sequence Synthetic primer
catgagcctacaaataataacagatgc

SEQ ID NO: 903 - DNA, 27 bp, Artificial Sequence
  Description of Artificial Sequence Synthetic primer
aaattagcttcacccatattacttagg

SEQ ID NO: 904 - DNA, 22 bp, Artificial Sequence
  Description of Artificial Sequence Synthetic primer
tgggatcaattagcaaagcagc

SEQ ID NO: 905 - DNA, 24 bp, Artificial Sequence
  Description of Artificial Sequence Synthetic primer
tccactcaaatcttagcatcatca

SEQ ID NO: 906 - DNA, 25 bp, Artificial Sequence
  Description of Artificial Sequence Synthetic primer
tggatgaaatgtttttgtaggacga

SEQ ID NO: 907 - DNA, 28 bp, Artificial Sequence
  Description of Artificial Sequence Synthetic primer
gaaaaggttgaggaattaaaagaagtgg

SEQ ID NO: 908 - DNA, 29 bp, Artificial Sequence
  Description of Artificial Sequence Synthetic primer
gtttcaaaatgataagacaatgccaaacg

SEQ ID NO: 909 - DNA, 23 bp, Artificial Sequence
  Description of Artificial Sequence Synthetic primer
aaaatgtttggcaagtgataccc

SEQ ID NO: 910 - DNA, 18 bp, Artificial Sequence
  Description of Artificial Sequence Synthetic primer
gggcctctctagggctcg

SEQ ID NO: 911 - DNA, 25 bp, Artificial Sequence
  Description of Artificial Sequence Synthetic primer
taggattgtggaattgtacctttgg

SEQ ID NO: 912 - DNA, 24 bp, Artificial Sequence
  Description of Artificial Sequence Synthetic primer
tgcatcataatcagtaattgaggc

SEQ ID NO: 913 - DNA, 27 bp, Artificial Sequence
  Description of Artificial Sequence Synthetic primer
aggacgttttgaattgttatgaagagt

SEQ ID NO: 914 - DNA, 28 bp, Artificial Sequence
  Description of Artificial Sequence Synthetic primer
tctttatgttacttctttttctagcact

SEQ ID NO: 915 - DNA, 26 bp, Artificial Sequence
  Description of Artificial Sequence Synthetic primer
actcaatcaagaatacatgcaaacat

SEQ ID NO: 916 - DNA, 24 bp, Artificial Sequence
  Description of Artificial Sequence Synthetic primer
gcacttgctcaaactcttcataac

SEQ ID NO: 917 - DNA, 23 bp, Artificial Sequence
  Description of Artificial Sequence Synthetic primer
tgtttgcatagaggactcatccc

SEQ ID NO: 918 - DNA, 21 bp, Artificial Sequence
  Description of Artificial Sequence Synthetic primer
tctttcgatctttggagagcc

SEQ ID NO: 919 - DNA, 22 bp, Artificial Sequence
  Description of Artificial Sequence Synthetic primer
gtttgcatagaggactcatccc

SEQ ID NO: 920 - DNA, 23 bp, Artificial Sequence
  Description of Artificial Sequence Synthetic primer
agttttcattccaaccaaagagc

SEQ ID NO: 921 - DNA, 22 bp, Artificial Sequence
  Description of Artificial Sequence Synthetic primer
tggtgtttgtgaagaagaagcc

SEQ ID NO: 922 - DNA, 21 bp, Artificial Sequence
  Description of Artificial Sequence Synthetic primer
ggtgaagctacttgagttggc

SEQ ID NO: 923 - DNA, 23 bp, Artificial Sequence
  Description of Artificial Sequence Synthetic primer
tttgtgaagccaactcaagtagc

SEQ ID NO: 924 - DNA, 23 bp, Artificial Sequence
  Description of Artificial Sequence Synthetic primer
attggatgaataagcaaagcagc

SEQ ID NO: 925 - DNA, 23 bp, Artificial Sequence
  Description of Artificial Sequence Synthetic primer
tcatcctttggaaaaccctttgg

SEQ ID NO: 926 - DNA, 21 bp, Artificial Sequence
  Description of Artificial Sequence Synthetic primer
aagcaaccgaaagcaaagagc

SEQ ID NO: 927 - DNA, 30 bp, Artificial Sequence
  Description of Artificial Sequence Synthetic primer
catattcgacaagtacaagaatgaaaaagg

SEQ ID NO: 928 - DNA, 22 bp, Artificial Sequence
  Description of Artificial Sequence Synthetic primer
ctccatgaactctcatttgtgc

SEQ ID NO: 929 - DNA, 24 bp, Artificial Sequence
  Description of Artificial Sequence Synthetic primer
ggagtgctatttctgggtttatgg

SEQ ID NO: 930 - DNA, 24 bp, Artificial Sequence
  Description of Artificial Sequence Synthetic primer
aggctctagttcttcaagtgtacc

SEQ ID NO: 931 - DNA, 20 bp, Artificial Sequence
  Description of Artificial Sequence Synthetic primer
gttggggttgatagtgcagg

SEQ ID NO: 932 - DNA, 27 bp, Artificial Sequence
  Description of Artificial Sequence Synthetic primer
tcattcataactcttccaactattgcc

SEQ ID NO: 933 - DNA, 21 bp, Artificial Sequence
  Description of Artificial Sequence Synthetic primer
ggagcggaagaaatcatcagg

SEQ ID NO: 934 - DNA, 22 bp, Artificial Sequence
  Description of Artificial Sequence Synthetic primer
tgggttgtgtaaagccatctcc

SEQ ID NO: 935 - DNA, 22 bp, Artificial Sequence
  Description of Artificial Sequence Synthetic primer
attcaacgattggggttgtctt

SEQ ID NO: 936 - DNA, 21 bp, Artificial Sequence
  Description of Artificial Sequence Synthetic primer
ccgaaagcaaagagcatcagc

SEQ ID NO: 937 - DNA, 30 bp, Artificial Sequence; identical to SEQ ID NO: 927
  Description of Artificial Sequence Synthetic primer

SEQ ID NO: 938 - DNA, 22 bp, Artificial Sequence
  Description of Artificial Sequence Synthetic primer
ctccatgaactctcatttgtgc

SEQ ID NO: 939 - DNA, 24 bp, Artificial Sequence
  Description of Artificial Sequence Synthetic primer
agaatagtggagtgctatttctgg

SEQ ID NO: 940 - DNA, 29 bp, Artificial Sequence
  Description of Artificial Sequence Synthetic primer
attattagtctgatttgggaagtttctgc

SEQ ID NO: 941 - DNA, 25 bp, Artificial Sequence
  Description of Artificial Sequence Synthetic primer
tggagtgctatttctgggtttatgg

SEQ ID NO: 942 - DNA, 27 bp, Artificial Sequence
  Description of Artificial Sequence Synthetic primer
atttggtgattattagtctgatttggg

SEQ ID NO: 943 - DNA, 20 bp, Artificial Sequence
  Description of Artificial Sequence Synthetic primer
gttggggttgatagtgcagg

SEQ ID NO: 944 - DNA, 27 bp, Artificial Sequence
  Description of Artificial Sequence Synthetic primer
tcattcataactcttccaactattgcc

SEQ ID NO: 945 - DNA, 22 bp, Artificial Sequence
  Description of Artificial Sequence Synthetic primer
gaaattgtggcttcaactgtgg

SEQ ID NO: 946 - DNA, 23 bp, Artificial Sequence
  Description of Artificial Sequence Synthetic primer
gagcaaagtgggttgtgtaaagc

SEQ ID NO: 947 - DNA, 25 bp, Artificial Sequence
  Description of Artificial Sequence Synthetic primer
agaaatcatcaggagaaattgtggc

SEQ ID NO: 948 - DNA, 22 bp, Artificial Sequence
  Description of Artificial Sequence Synthetic primer
tgggttgtgtaaagccatctcc

SEQ ID NO: 949 - DNA, 29 bp, Artificial Sequence
  Description of Artificial Sequence Synthetic primer
cacacaaatcttagtatcttcaaatgaca

SEQ ID NO: 950 - DNA, 27 bp, Artificial Sequence
  Description of Artificial Sequence Synthetic primer
caaatcttgttgtgaaatgttgtaatg

SEQ ID NO: 951 - DNA, 27 bp, Artificial Sequence
  Description of Artificial Sequence Synthetic primer
aaaaggttgaggaattaaaagaagtgg

SEQ ID NO: 952 - DNA, 30 bp, Artificial Sequence
  Description of Artificial Sequence Synthetic primer
agtttcaaaatgataagacaatcccaaacg

SEQ ID NO: 953 - DNA, 25 bp, Artificial Sequence
  Description of Artificial Sequence Synthetic primer
tgaaaaattcaaagacgaggatggg

SEQ ID NO: 954 - DNA, 25 bp, Artificial Sequence
  Description of Artificial Sequence Synthetic primer
catttaggtgtgttgttgtgaaagc

SEQ ID NO: 955 - DNA, 23 bp, Artificial Sequence
  Description of Artificial Sequence Synthetic primer
acttgctttcacaacaacacacc

SEQ ID NO: 956 - DNA, 20 bp, Artificial Sequence
  Description of Artificial Sequence Synthetic primer
aagggcctctctagggctcg

SEQ ID NO: 957 - DNA, 21 bp, Artificial Sequence
  Description of Artificial Sequence Synthetic primer
acaccatgaagacgatgatcc

SEQ ID NO: 958 - DNA, 23 bp, Artificial Sequence
  Description of Artificial Sequence Synthetic primer
acctgctgacttcactaagttcc

SEQ ID NO: 959 - DNA, 28 bp, Artificial Sequence
  Description of Artificial Sequence Synthetic primer
agataggattgtggaattatacctttgg

SEQ ID NO: 960 - DNA, 23 bp, Artificial Sequence
  Description of Artificial Sequence Synthetic primer
agctcaagttcttcaagtgtacc

SEQ ID NO: 961 - DNA, 26 bp, Artificial Sequence
  Description of Artificial Sequence Synthetic primer
tttgaattgttatgaagagtttgagc

SEQ ID NO: 962 - DNA, 27 bp, Artificial Sequence
  Description of Artificial Sequence Synthetic primer
tgtattgttcttctttttctagcactt

SEQ ID NO: 963 - DNA, 24 bp, Artificial Sequence
  Description of Artificial Sequence Synthetic primer
atttggatgaagctcgatgtttgc

SEQ ID NO: 964 - DNA, 28 bp, Artificial Sequence
  Description of Artificial Sequence Synthetic primer
ccatgagcctacaaatagtaacagatgc

SEQ ID NO: 965 - DNA, 20 bp, Artificial Sequence
  Description of Artificial Sequence Synthetic primer
tgaggaacattcagcagtgg

SEQ ID NO: 966 - DNA, 30 bp, Artificial Sequence
  Description of Artificial Sequence Synthetic primer
gcttcaaaaactcttcatttatttctttcc

SEQ ID NO: 967 - DNA, 28 bp, Artificial Sequence
  Description of Artificial Sequence Synthetic primer
aagaaataaatgaagagtttttgaagcc

SEQ ID NO: 968 - DNA, 23 bp, Artificial Sequence
  Description of Artificial Sequence Synthetic primer
ccaacgtgtgtataaccatctcc

SEQ ID NO: 969 - DNA, 24 bp, Artificial Sequence
  Description of Artificial Sequence Synthetic primer
atgtcactatcaggactaatctcc

SEQ ID NO: 970 - DNA, 21 bp, Artificial Sequence
  Description of Artificial Sequence Synthetic primer
aaaatgagcatcccacaatgg

SEQ ID NO: 971 - DNA, 25 bp, Artificial Sequence
  Description of Artificial Sequence Synthetic primer
agctatgggaaaagaatcaatgagc

SEQ ID NO: 972 - DNA, 23 bp, Artificial Sequence
  Description of Artificial Sequence Synthetic primer
gagtttgaaatgaagagcagtgg

SEQ ID NO: 973 - DNA, 22 bp, Artificial Sequence
  Description of Artificial Sequence Synthetic primer
gacgagaaaaaggagttcaagg

SEQ ID NO: 974 - DNA, 27 bp, Artificial Sequence
  Description of Artificial Sequence Synthetic primer
tcatcaagtattgtttgagatgtttgg

SEQ ID NO: 975 - DNA, 25 bp, Artificial Sequence
  Description of Artificial Sequence Synthetic primer
gccgaaaaatgctcacaaagattgg

SEQ ID NO: 976 - DNA, 24 bp, Artificial Sequence
  Description of Artificial Sequence Synthetic primer
cttcccaatgcgtgttgaaagagg

SEQ ID NO: 977 - DNA, 26 bp, Artificial Sequence
  Description of Artificial Sequence Synthetic primer
accaacttccagattacatgaagata

SEQ ID NO: 978 - DNA, 20 bp, Artificial Sequence
  Description of Artificial Sequence Synthetic primer
tagcacgtcatacgccattt

SEQ ID NO: 979 - DNA, 30 bp, Artificial Sequence
  Description of Artificial Sequence Synthetic primer
gaacccttaattctagtcaatctttattgc

SEQ ID NO: 980 - DNA, 25 bp, Artificial Sequence
  Description of Artificial Sequence Synthetic primer
ttcctaaatcatcaacaagtcgagc

SEQ ID NO: 981 - DNA, 24 bp, Artificial Sequence
  Description of Artificial Sequence Synthetic primer
tgaactgaaaagaggagacaatcc

SEQ ID NO: 982 - DNA, 20 bp, Artificial Sequence
  Description of Artificial Sequence Synthetic primer
aatggagactcacccactcg

SEQ ID NO: 983 - DNA, 23 bp, Artificial Sequence
  Description of Artificial Sequence Synthetic primer
gccatagatttcgttaggacagc

SEQ ID NO: 984 - DNA, 23 bp, Artificial Sequence
  Description of Artificial Sequence Synthetic primer
gggaatggaagtgaagaacaagg

SEQ ID NO: 985 - DNA, 24 bp, Artificial Sequence
  Description of Artificial Sequence Synthetic primer
tcaaagaacaaccagcaatagtcc

SEQ ID NO: 986 - DNA, 24 bp, Artificial Sequence
  Description of Artificial Sequence Synthetic primer
ttggagtgattggataaaatgagc

SEQ ID NO: 987 - DNA, 24 bp, Artificial Sequence
  Description of Artificial Sequence Synthetic primer
gctatgggaaaagaatcaatgagc

SEQ ID NO: 988 - DNA, 22 bp, Artificial Sequence
  Description of Artificial Sequence Synthetic primer
agtttgaaatgaagagcagtgg

SEQ ID NO: 989 - DNA, 24 bp, Artificial Sequence
  Description of Artificial Sequence Synthetic primer
tcttgagaaggaggctgaaaatcc

SEQ ID NO: 990 - DNA, 24 bp, Artificial Sequence
  Description of Artificial Sequence Synthetic primer
ggagtttgaaatgaagagcagtgg

SEQ ID NO: 991 - DNA, 27 bp, Artificial Sequence
  Description of Artificial Sequence Synthetic primer
ggagagtttgagtaaagatgtgaaagg

SEQ ID NO: 992 - DNA, 24 bp, Artificial Sequence
  Description of Artificial Sequence Synthetic primer
tccaaccttttcattctccaatgc

SEQ ID NO: 993 - DNA, 26 bp, Artificial Sequence
  Description of Artificial Sequence Synthetic primer
tttgagtaaagatgtgaaaggaatgg

SEQ ID NO: 994 - DNA, 25 bp, Artificial Sequence
  Description of Artificial Sequence Synthetic primer
gatgtttggttgtgaaatctcttgc

SEQ ID NO: 995 - DNA, 23 bp, Artificial Sequence
  Description of Artificial Sequence Synthetic primer
gagccacagtttagatattgccg

SEQ ID NO: 996 - DNA, 21 bp, Artificial Sequence
  Description of Artificial Sequence Synthetic primer
agaggcttagctcatcaagcg

SEQ ID NO: 997 - DNA, 25 bp, Artificial Sequence
  Description of Artificial Sequence Synthetic primer
cgaccaacttccagattacatgaag

SEQ ID NO: 998 - DNA, 21 bp, Artificial Sequence
  Description of Artificial Sequence Synthetic primer
agcacgtcatacgccatttca

SEQ ID NO: 999 - DNA, 25 bp, Artificial Sequence
  Description of Artificial Sequence Synthetic primer
tggaggcaaattggtatcatagtgg

SEQ ID NO: 1000 - DNA, 23 bp, Artificial Sequence
  Description of Artificial Sequence Synthetic primer
aaaggtgggatattgaagcaagc

SEQ ID NO: 1001 - DNA, 30 bp, Artificial Sequence; identical to SEQ ID NO: 979
  Description of Artificial Sequence Synthetic primer

SEQ ID NO: 1002 - DNA, 25 bp, Artificial Sequence
  Description of Artificial Sequence Synthetic primer
ttcctaaatcatcaacaagtcgagc

SEQ ID NO: 1003 - DNA, 24 bp, Artificial Sequence
  Description of Artificial Sequence Synthetic primer
tgaactgaaaagaggagacaatcc

SEQ ID NO: 1004 - DNA, 20 bp, Artificial Sequence
  Description of Artificial Sequence Synthetic primer
aatggagactcacccactcg

SEQ ID NO: 1005 - DNA, 23 bp, Artificial Sequence
  Description of Artificial Sequence Synthetic primer
tggaaggaaatgaatgaagctcg

SEQ ID NO: 1006 - DNA, 21 bp, Artificial Sequence
  Description of Artificial Sequence Synthetic primer
accccatcttgctccttttgg

SEQ ID NO: 1007 - DNA, 23 bp, Artificial Sequence
  Description of Artificial Sequence Synthetic primer
gagcttgactatgttgaaaaccc

SEQ ID NO: 1008 - DNA, 24 bp, Artificial Sequence
  Description of Artificial Sequence Synthetic primer
ataaccttgttgtctaaccaatcg

SEQ ID NO: 1009 - DNA, 23 bp, Artificial Sequence
  Description of Artificial Sequence Synthetic primer
gcttcacaactaagagtgcatgg

SEQ ID NO: 1010 - DNA, 24 bp, Artificial Sequence
  Description of Artificial Sequence Synthetic primer
taatgtctagcctgtctcctttgc

SEQ ID NO: 1011 - DNA, 24 bp, Artificial Sequence
  Description of Artificial Sequence Synthetic primer
aacgatattcgaggaatgttaagc

SEQ ID NO: 1012 - DNA, 23 bp, Artificial Sequence
  Description of Artificial Sequence Synthetic primer
aggttttagctccagattcgagg

SEQ ID NO: 1013 - DNA, 28 bp, Artificial Sequence
  Description of Artificial Sequence Synthetic primer
taatggattgttacttttggacttttgg

SEQ ID NO: 1014 - DNA, 26 bp, Artificial Sequence
  Description of Artificial Sequence Synthetic primer
gtattgcttcagttaagaggtgtagc

SEQ ID NO: 1015 - DNA, 24 bp, Artificial Sequence
  Description of Artificial Sequence Synthetic primer
tcaactaccagagtacattcaacc

SEQ ID NO: 1016 - DNA, 30 bp, Artificial Sequence
  Description of Artificial Sequence Synthetic primer
gcaatatgatttgtccttagtaaattctcc

SEQ ID NO: 1017 - DNA, 22 bp, Artificial Sequence
  Description of Artificial Sequence Synthetic primer
cacttgttactgctgcttctcc

SEQ ID NO: 1018 - DNA, 24 bp, Artificial Sequence
  Description of Artificial Sequence Synthetic primer
gccaatgtcgttaagaactctacc

SEQ ID NO: 1019 - DNA, 25 bp, Artificial Sequence
  Description of Artificial Sequence Synthetic primer
tctgaatgaagagtgtctctatccc

SEQ ID NO: 1020 - DNA, 24 bp, Artificial Sequence
  Description of Artificial Sequence Synthetic primer
agcttctcttgtaaaaccatctcc

SEQ ID NO: 1021 - DNA, 24 bp, Artificial Sequence
  Description of Artificial Sequence Synthetic primer
atcatcgaaattgaaagacacagg

SEQ ID NO: 1022 - DNA, 20 bp, Artificial Sequence
  Description of Artificial Sequence Synthetic primer
gggcaagatagttgctctgc

SEQ ID NO: 1023 - DNA, 21 bp, Artificial Sequence
  Description of Artificial Sequence Synthetic primer
gagcaactatcttgcccaacc

SEQ ID NO: 1024 - DNA, 27 bp, Artificial Sequence
  Description of Artificial Sequence Synthetic primer
tcatatttgttgttaagagactccagg

SEQ ID NO: 1025 - DNA, 21 bp, Artificial Sequence
  Description of Artificial Sequence Synthetic primer
agcttatcgaggatgtgaggc

SEQ ID NO: 1026 - DNA, 20 bp, Artificial Sequence
  Description of Artificial Sequence Synthetic primer
tccatgaagcctaagaagcc

SEQ ID NO: 1027 - DNA, 24 bp, Artificial Sequence
  Description of Artificial Sequence Synthetic primer
tgtttcacaaggcatatttgttgg

SEQ ID NO: 1028 - DNA, 21 bp, Artificial Sequence
  Description of Artificial Sequence Synthetic primer
agttccaaagcatgaaccacc

SEQ ID NO: 1029 - DNA, 20 bp, Artificial Sequence
  Description of Artificial Sequence Synthetic primer
gttggtggaagaatgtgggc

SEQ ID NO: 1030 - DNA, 22 bp, Artificial Sequence
  Description of Artificial Sequence Synthetic primer
ggcattggtgaagtgtctgagc

SEQ ID NO: 1031 - DNA, 24 bp, Artificial Sequence
  Description of Artificial Sequence Synthetic primer
tgtacgggaaactgaaaaacttcc

SEQ ID NO: 1032 - DNA, 28 bp, Artificial Sequence
  Description of Artificial Sequence Synthetic primer
ccctttagatgaggtaaaactaacttgc

SEQ ID NO: 1033 - DNA, 21 bp, Artificial Sequence
  Description of Artificial Sequence Synthetic primer
gcttggatttcatcttcgggc

SEQ ID NO: 1034 - DNA, 23 bp, Artificial Sequence
  Description of Artificial Sequence Synthetic primer
cgctgaagttcctaaatcgttgc

SEQ ID NO: 1035 - DNA, 27 bp, Artificial Sequence
  Description of Artificial Sequence Synthetic primer
cctctcattcccaataagtaaactagc

SEQ ID NO: 1036 - DNA, 23 bp, Artificial Sequence
  Description of Artificial Sequence Synthetic primer
tcctcaaagtacaggagacatcg

SEQ ID NO: 1037 - DNA, 22 bp, Artificial Sequence
  Description of Artificial Sequence Synthetic primer
ttgatgctattcaacggctagg

SEQ ID NO: 1038 - DNA, 20 bp, Artificial Sequence
  Description of Artificial Sequence Synthetic primer
cagcaggaacgaagtgaccg

SEQ ID NO: 1039 - DNA, 22 bp, Artificial Sequence
  Description of Artificial Sequence Synthetic primer
atatgaagcctcccatctatgc

SEQ ID NO: 1040 - DNA, 23 bp, Artificial Sequence
  Description of Artificial Sequence Synthetic primer
tgggatagacaccaaaaaggtcc

SEQ ID NO: 1041 - DNA, 22 bp, Artificial Sequence
  Description of Artificial Sequence Synthetic primer
ggtggcgagacattggtttagc

SEQ ID NO: 1042 - DNA, 22 bp, Artificial Sequence
  Description of Artificial Sequence Synthetic primer
atggattttgtaagcgcaaccc

SEQ ID NO: 1043 - DNA, 26 bp, Artificial Sequence
  Description of Artificial Sequence Synthetic primer
tatagaaaaacttccagactccatga

SEQ ID NO: 1044 - DNA, 19 bp, Artificial Sequence
  Description of Artificial Sequence Synthetic primer
taaagggctccatccacgc

SEQ ID NO: 1045 - DNA, 19 bp, Artificial Sequence
  Description of Artificial Sequence Synthetic primer
gggcaagtttgtgcgaagc

SEQ ID NO: 1046 - DNA, 21 bp, Artificial Sequence
  Description of Artificial Sequence Synthetic primer
tggcactaccaaagtcatccc

SEQ ID NO: 1047 - DNA, 23 bp, Artificial Sequence
  Description of Artificial Sequence Synthetic primer
aggacatgacggatcttatgtgg

SEQ ID NO: 1048 - DNA, 20 bp, Artificial Sequence
  Description of Artificial Sequence Synthetic primer
aggaatggtggtggaaacgc

SEQ ID NO: 1049 - DNA, 21 bp, Artificial Sequence
  Description of Artificial Sequence Synthetic primer
tccaccaccattcctcaaagc

SEQ ID NO: 1050 - DNA, 22 bp, Artificial Sequence
  Description of Artificial Sequence Synthetic primer
atgttcttccaagtggggtagg

SEQ ID NO: 1051 - DNA, 20 bp, Artificial Sequence
  Description of Artificial Sequence Synthetic primer
gaacttggtcaacgctgtgc

SEQ ID NO: 1052 - DNA, 22 bp, Artificial Sequence
  Description of Artificial Sequence Synthetic primer
acctccttgtctcagtaatcgg

SEQ ID NO: 1053 - DNA, 22 bp, Artificial Sequence
  Description of Artificial Sequence Synthetic primer
tagtggaagaagacagagaggg

SEQ ID NO: 1054 - DNA, 22 bp, Artificial Sequence
  Description of Artificial Sequence Synthetic primer
agtgttggctgagaaactgtcc

SEQ ID NO: 1055 - DNA, 20 bp, Artificial Sequence
  Description of Artificial Sequence Synthetic primer
tctcagccaacactgctacc

SEQ ID NO: 1056 - DNA, 24 bp, Artificial Sequence
  Description of Artificial Sequence Synthetic primer
gagacttgaacaatttccctttgg

SEQ ID NO: 1057 - DNA, 21 bp, Artificial Sequence
  Description of Artificial Sequence Synthetic primer
ggtggaaagagcttggtttgg

SEQ ID NO: 1058 - DNA, 21 bp, Artificial Sequence
  Description of Artificial Sequence Synthetic primer
gagtgagttcgtctagtgtcc

SEQ ID NO: 1059 - DNA, 24 bp, Artificial Sequence
  Description of Artificial Sequence Synthetic primer
gggaaattaaagagcagttacccg

SEQ ID NO: 1060 - DNA, 22 bp, Artificial Sequence
  Description of Artificial Sequence Synthetic primer
gggttccatccatgttttctgt

SEQ ID NO: 1061 - DNA, 21 bp, Artificial Sequence
  Description of Artificial Sequence Synthetic primer
ggtttggttgtgggaagttgc

SEQ ID NO: 1062 - DNA, 21 bp, Artificial Sequence
  Description of Artificial Sequence Synthetic primer
ctgctgtagaagacacaaggc

SEQ ID NO: 1063 - DNA, 21 bp, Artificial Sequence
  Description of Artificial Sequence Synthetic primer
gaacgcgattgtgagttctgg

SEQ ID NO: 1064 - DNA, 20 bp, Artificial Sequence
  Description of Artificial Sequence Synthetic primer
ggcacttcctaagtcatccc

SEQ ID NO: 1065 - DNA, 21 bp, Artificial Sequence
  Description of Artificial Sequence Synthetic primer
atgggcatgatgggtcttacg

SEQ ID NO: 1066 - DNA, 20 bp, Artificial Sequence
  Description of Artificial Sequence Synthetic primer
ctccttattgaggcgttccc

SEQ ID NO: 1067 - DNA, 20 bp, Artificial Sequence
  Description of Artificial Sequence Synthetic primer
gcaagggagcaagtgattcg

SEQ ID NO: 1068 - DNA, 18 bp, Artificial Sequence
  Description of Artificial Sequence Synthetic primer
ctggaggcttggaaggcg

SEQ ID NO: 1069 - DNA, 23 bp, Artificial Sequence
  Description of Artificial Sequence Synthetic primer
actcggctactttctcaaagagg

SEQ ID NO: 1070 - DNA, 23 bp, Artificial Sequence
  Description of Artificial Sequence Synthetic primer
atggattaccaaaacgaggaagc

SEQ ID NO: 1071 - DNA, 24 bp, Artificial Sequence
  Description of Artificial Sequence Synthetic primer
aggctcatagatagcatccaacgg

SEQ ID NO: 1072 - DNA, 20 bp, Artificial Sequence
  Description of Artificial Sequence Synthetic primer
gtagccgaaaacgaagcccc

SEQ ID NO: 1073 - DNA, 29 bp, Artificial Sequence
  Description of Artificial Sequence Synthetic primer
tccttcagatgttgtcagatttcaattcc

SEQ ID NO: 1074 - DNA, 24 bp, Artificial Sequence
  Description of Artificial Sequence Synthetic primer
gaggtagttggaaagccattatgc

SEQ ID NO: 1075 - DNA, 20 bp, Artificial Sequence
  Description of Artificial Sequence Synthetic primer
atgctgagcttgtatgaggc

SEQ ID NO: 1076 - DNA, 20 bp, Artificial Sequence
  Description of Artificial Sequence Synthetic primer
caagaagagcagggcagtgg

SEQ ID NO: 1077 - DNA, 19 bp, Artificial Sequence
  Description of Artificial Sequence Synthetic primer
gacacctgaggatggcacc

SEQ ID NO: 1078 - DNA, 21 bp, Artificial Sequence
  Description of Artificial Sequence Synthetic primer
ttgtggagtgactgaagctcg

SEQ ID NO: 1079 - DNA, 20 bp, Artificial Sequence
  Description of Artificial Sequence Synthetic primer
gtggaaacagttgggtctgg

SEQ ID NO: 1080 - DNA, 22 bp, Artificial Sequence
  Description of Artificial Sequence Synthetic primer
gtggagttcatccaaagatccg

SEQ ID NO: 1081 - DNA, 21 bp, Artificial Sequence
  Description of Artificial Sequence Synthetic primer
gatgggatcttggtgcaatgg

SEQ ID NO: 1082 - DNA, 25 bp, Artificial Sequence
  Description of Artificial Sequence Synthetic primer
acatattttcatgtactcaggaagc

SEQ ID NO: 1083 - DNA, 23 bp, Artificial Sequence
  Description of Artificial Sequence Synthetic primer
tgaaattggctacagagttctca

SEQ ID NO: 1084 - DNA, 23 bp, Artificial Sequence
  Description of Artificial Sequence Synthetic primer
agtgttgtgtaacgcataatcca

SEQ ID NO: 1085 - DNA, 24 bp, Artificial Sequence
  Description of Artificial Sequence Synthetic primer
gcatttctaaccgaagcagaatgg

SEQ ID NO: 1086 - DNA, 21 bp, Artificial Sequence
  Description of Artificial Sequence Synthetic primer
tggctgttcccaaatcatccc

SEQ ID NO: 1087 - DNA, 26 bp, Artificial Sequence
  Description of Artificial Sequence Synthetic primer
cactcatttttcctcataggtcatgg

SEQ ID NO: 1088 - DNA, 22 bp, Artificial Sequence
  Description of Artificial Sequence Synthetic primer
ttggctgttcccaaatcatccc

SEQ ID NO: 1089 - DNA, 23 bp, Artificial Sequence
  Description of Artificial Sequence Synthetic primer
agagaggagatgttgcttctagc

SEQ ID NO: 1090 - DNA, 20 bp, Artificial Sequence
  Description of Artificial Sequence Synthetic primer
aaggtcaaaacaggccgtgg

SEQ ID NO: 1091 - DNA, 22 bp, Artificial Sequence
  Description of Artificial Sequence Synthetic primer
agctcgtggatagagctaaacg

SEQ ID NO: 1092 - DNA, 23 bp, Artificial Sequence
  Description of Artificial Sequence Synthetic primer
ccatgttgatagatgacttgggc

SEQ ID NO: 1093 - DNA, 21 bp, Artificial Sequence
  Description of Artificial Sequence Synthetic primer
atgctaccccatccaatgtgc

SEQ ID NO: 1094 - DNA, 28 bp, Artificial Sequence
  Description of Artificial Sequence Synthetic primer
aatataatcgaaagaccaaatggagggc

SEQ ID NO: 1095 - DNA, 26 bp, Artificial Sequence
  Description of Artificial Sequence Synthetic primer
ttagttgagatggaaaactctttagc

SEQ ID NO: 1096 - DNA, 22 bp, Artificial Sequence
  Description of Artificial Sequence Synthetic primer
atagccatgttgacgtagaagc

SEQ ID NO: 1097 - DNA, 20 bp, Artificial Sequence
  Description of Artificial Sequence Synthetic primer
tcccacttcatcggaggacc

SEQ ID NO: 1098 - DNA, 23 bp, Artificial Sequence
  Description of Artificial Sequence Synthetic primer
ttggctaactcaagcaacatagg

SEQ ID NO: 1099 - DNA, 25 bp, Artificial Sequence
  Description of Artificial Sequence Synthetic primer
ggtggagacatactaaacttggaga

SEQ ID NO: 1100 - DNA, 25 bp, Artificial Sequence
  Description of Artificial Sequence Synthetic primer
gctttggtgaaaagctctaattcat

SEQ ID NO: 1101 - DNA, 27 bp, Artificial Sequence
  Description of Artificial Sequence Synthetic primer
atgagttaccagaatacatgaagatgc

SEQ ID NO: 1102 - DNA, 29 bp, Artificial Sequence
  Description of Artificial Sequence Synthetic primer
ggtattgaatgttgatggagatttcttgg

SEQ ID NO: 1103 - DNA, 26 bp, Artificial Sequence
  Description of Artificial Sequence Synthetic primer
ggtcgatatgtgtaaaagtttcttgc

SEQ ID NO: 1104 - DNA, 22 bp, Artificial Sequence
  Description of Artificial Sequence Synthetic primer
actggtgctcctactgaaatcc

SEQ ID NO: 1105 - DNA, 23 bp, Artificial Sequence
  Description of Artificial Sequence Synthetic primer
aatggttggatttcagtaggagc

SEQ ID NO: 1106 - DNA, 21 bp, Artificial Sequence
  Description of Artificial Sequence Synthetic primer
tggcactgtgacgaataatgg

SEQ ID NO: 1107 - DNA, 22 bp, Artificial Sequence
  Description of Artificial Sequence Synthetic primer
attgaaaagaggtgatgctccg

SEQ ID NO: 1108 - DNA, 29 bp, Artificial Sequence
  Description of Artificial Sequence Synthetic primer
ttggattgattatcttgagaactatgacc

SEQ ID NO: 1109 - DNA, 23 bp, Artificial Sequence
  Description of Artificial Sequence Synthetic primer
attgtatctgaagaggaagctcg

SEQ ID NO: 1110 - DNA, 28 bp, Artificial Sequence
  Description of Artificial Sequence Synthetic primer
tataagggaataggttcaataatcaagg

SEQ ID NO: 1111 - DNA, 22 bp, Artificial Sequence
  Description of Artificial Sequence Synthetic primer
atgtgctgtggtcaatagttct

SEQ ID NO: 1112 - DNA, 21 bp, Artificial Sequence
  Description of Artificial Sequence Synthetic primer
aaagaccaaatggagggctca

SEQ ID NO: 1113 - DNA, 22 bp, Artificial Sequence
  Description of Artificial Sequence Synthetic primer
acaggtcgagtcaaagaattgg

SEQ ID NO: 1114 - DNA, 20 bp, Artificial Sequence
  Description of Artificial Sequence Synthetic primer
gccatgttgacgtagaagcc

SEQ ID NO: 1115 - DNA, 21 bp, Artificial Sequence
  Description of Artificial Sequence Synthetic primer
ctcccacttcattggaggact

SEQ ID NO: 1116 - DNA, 24 bp, Artificial Sequence
  Description of Artificial Sequence Synthetic primer
agcaaactcaagcaaaataggatt

SEQ ID NO: 1117 - DNA, 20 bp, Artificial Sequence
  Description of Artificial Sequence Synthetic primer
gctaagtggttcatcgacgc

SEQ ID NO: 1118 - DNA, 25 bp, Artificial Sequence
  Description of Artificial Sequence Synthetic primer
tgatgtgttgattgtatcatgttga

SEQ ID NO: 1119 - DNA, 22 bp, Artificial Sequence
  Description of Artificial Sequence Synthetic primer
ggtggaggcatactaaacttgg

SEQ ID NO: 1120 - DNA, 27 bp, Artificial Sequence
  Description of Artificial Sequence Synthetic primer
gaaaagctctaattcatccaatgttcc

SEQ ID NO: 1121 - DNA, 27 bp, Artificial Sequence
  Description of Artificial Sequence Synthetic primer
gatgggatatggaaatgataaatgagt

SEQ ID NO: 1122 - DNA, 23 bp, Artificial Sequence
  Description of Artificial Sequence Synthetic primer
tgttgatggagatgtgttggtct

SEQ ID NO: 1123 - DNA, 19 bp, Artificial Sequence
  Description of Artificial Sequence Synthetic primer
gatttcagtgggagcaccg

SEQ ID NO: 1124 - DNA, 21 bp, Artificial Sequence
  Description of Artificial Sequence Synthetic primer
actatgacgaattatggcggg

SEQ ID NO: 1125 - DNA, 23 bp, Artificial Sequence
  Description of Artificial Sequence Synthetic primer
gaactgaaaagaggtgatgctcc

SEQ ID NO: 1126 - DNA, 30 bp, Artificial Sequence; identical to SEQ ID NO: 472
  Description of Artificial Sequence Synthetic primer

SEQ ID NO: 1127 - DNA, 21 bp, Artificial Sequence
  Description of Artificial Sequence Synthetic primer
atgctaccccatccaatgtgc

SEQ ID NO: 1128 - DNA, 28 bp, Artificial Sequence
  Description of Artificial Sequence Synthetic primer
aatataatcaaaagaccaaatggagggc

SEQ ID NO: 1129 - DNA, 25 bp, Artificial Sequence
  Description of Artificial Sequence Synthetic primer
caacttgaactcattgatacattgc

SEQ ID NO: 1130 - DNA, 22 bp, Artificial Sequence
  Description of Artificial Sequence Synthetic primer
tagccatgttgacgtagaagcc

SEQ ID NO: 1131 - DNA, 20 bp, Artificial Sequence
  Description of Artificial Sequence Synthetic primer
agctcccacttcatcggagg

SEQ ID NO: 1132 - DNA, 24 bp, Artificial Sequence
  Description of Artificial Sequence Synthetic primer
tttggctaactcaagcaacatagg

SEQ ID NO: 1133 - DNA, 25 bp, Artificial Sequence
  Description of Artificial Sequence Synthetic primer
ggtggagacatactaaacttggaga

SEQ ID NO: 1134 - DNA, 25 bp, Artificial Sequence
  Description of Artificial Sequence Synthetic primer
gctttggtgaaaagctctaattcat

SEQ ID NO: 1135 - DNA, 25 bp, Artificial Sequence
  Description of Artificial Sequence Synthetic primer
agttaccagaatacatgaagatgcc

SEQ ID NO: 1136 - DNA, 24 bp, Artificial Sequence
  Description of Artificial Sequence Synthetic primer
ttcttggtctcttaacacctcaaa

SEQ ID NO: 1137 - DNA, 26 bp, Artificial Sequence
  Description of Artificial Sequence Synthetic primer
ggtcgatatgtgtaaaagtttcttgc

SEQ ID NO: 1138 - DNA, 22 bp, Artificial Sequence
  Description of Artificial Sequence Synthetic primer
actggtgctcctactgaaatcc

SEQ ID NO: 1139 - DNA, 26 bp, Artificial Sequence
  Description of Artificial Sequence Synthetic primer
gaaaatggttggatttcagtaggagc

SEQ ID NO: 1140 - DNA, 22 bp, Artificial Sequence
  Description of Artificial Sequence Synthetic primer
tggcactgtgacgaataatggc

SEQ ID NO: 1141 - DNA, 23 bp, Artificial Sequence
  Description of Artificial Sequence Synthetic primer
aattgaaaagaggtgatgctccg

SEQ ID NO: 1142 - DNA, 30 bp, Artificial Sequence; identical to SEQ ID NO: 472
  Description of Artificial Sequence Synthetic primer

SEQ ID NO: 1143 - DNA, 23 bp, Artificial Sequence
  Description of Artificial Sequence Synthetic primer
agtcaagtgttagcttcatctca

SEQ ID NO: 1144 - DNA, 20 bp, Artificial Sequence
  Description of Artificial Sequence Synthetic primer
cgccccaaatagaagggtga

SEQ ID NO: 1145 - DNA, 25 bp, Artificial Sequence
  Description of Artificial Sequence Synthetic primer
gagagtgaaattgagaaattgttgg

SEQ ID NO: 1146 - DNA, 29 bp, Artificial Sequence
  Description of Artificial Sequence Synthetic primer
tgtcttaataatctaaaccaaagagaagc

SEQ ID NO: 1147 - DNA, 19 bp, Artificial Sequence
  Description of Artificial Sequence Synthetic primer
tgataaccgacgtttcggg

SEQ ID NO: 1148 - DNA, 22 bp, Artificial Sequence
  Description of Artificial Sequence Synthetic primer
ctagcatgaagcctctctaagg

SEQ ID NO: 1149 - DNA, 22 bp, Artificial Sequence
  Description of Artificial Sequence Synthetic primer
ggaaaggccactaagaatgacc

SEQ ID NO: 1150 - DNA, 23 bp, Artificial Sequence
  Description of Artificial Sequence Synthetic primer
acctcacaatttcactaagctcc

SEQ ID NO: 1151 - DNA, 21 bp, Artificial Sequence
  Description of Artificial Sequence Synthetic primer
gtggaaggagcatgagtttgc

SEQ ID NO: 1152 - DNA, 25 bp, Artificial Sequence
  Description of Artificial Sequence Synthetic primer
tgactttggttagaagttttcttgc

SEQ ID NO: 1153 - DNA, 26 bp, Artificial Sequence
  Description of Artificial Sequence Synthetic primer
gaagaagctcgatggttaaatgaagg

SEQ ID NO: 1154 - DNA, 25 bp, Artificial Sequence
  Description of Artificial Sequence Synthetic primer
agtagaagctgaaacaatcttaggg

SEQ ID NO: 1155 - DNA, 23 bp, Artificial Sequence
  Description of Artificial Sequence Synthetic primer
tctggttacgttttgttgatagc

SEQ ID NO: 1156 - DNA, 25 bp, Artificial Sequence
  Description of Artificial Sequence Synthetic primer
tgaatcttgagaggagagtagaagc

SEQ ID NO: 1157 - DNA, 24 bp, Artificial Sequence
  Description of Artificial Sequence Synthetic primer
tgaagcaatatgaggtttcagagg

SEQ ID NO: 1158 - DNA, 25 bp, Artificial Sequence
  Description of Artificial Sequence Synthetic primer
atgggatgggatctataagtaaagc

SEQ ID NO: 1159 - DNA, 29 bp, Artificial Sequence
  Description of Artificial Sequence Synthetic primer
aagtcttagcttcatctcaattatgtgac

SEQ ID NO: 1160 - DNA, 22 bp, Artificial Sequence
  Description of Artificial Sequence Synthetic primer
tcgatcaccccaaatagaaggg

SEQ ID NO: 1161 - DNA, 26 bp, Artificial Sequence
  Description of Artificial Sequence Synthetic primer
cgagagtgaaatcgagaaattattgg

SEQ ID NO: 1162 - DNA, 25 bp, Artificial Sequence
  Description of Artificial Sequence Synthetic primer
cactttgtcttaatagtctgaaccg

SEQ ID NO: 1163 - DNA, 19 bp, Artificial Sequence
  Description of Artificial Sequence Synthetic primer
agcttgtatgaggcttcgc

SEQ ID NO: 1164 - DNA, 24 bp, Artificial Sequence
  Description of Artificial Sequence Synthetic primer
tctctatggtctttcttaatggcg

SEQ ID NO: 1165 - DNA, 27 bp, Artificial Sequence
  Description of Artificial Sequence Synthetic primer
ggtggaactgtatttttggatattggg

SEQ ID NO: 1166 - DNA, 26 bp, Artificial Sequence
  Description of Artificial Sequence Synthetic primer
acctttgaattgctttggtaagaagc

SEQ ID NO: 1167 - DNA, 23 bp, Artificial Sequence
  Description of Artificial Sequence Synthetic primer
gaagctcgatggttgaatgaagg

SEQ ID NO: 1168 - DNA, 19 bp, Artificial Sequence
  Description of Artificial Sequence Synthetic primer
gcgagagcctatgtcatcc

SEQ ID NO: 1169 - DNA, 24 bp, Artificial Sequence
  Description of Artificial Sequence Synthetic primer
tgtggttacgttatgttgatagcc

SEQ ID NO: 1170 - DNA, 23 bp, Artificial Sequence
  Description of Artificial Sequence Synthetic primer
aatcttgataggagagtggaagc

SEQ ID NO: 1171 - DNA, 27 bp, Artificial Sequence
  Description of Artificial Sequence Synthetic primer
gtttgagcaagagagaaatcacatacc

SEQ ID NO: 1172 - DNA, 30 bp, Artificial Sequence
  Description of Artificial Sequence Synthetic primer
tgtaaaattaagaacacgaactaagatagg

SEQ ID NO: 1173 - DNA, 28 bp, Artificial Sequence
  Description of Artificial Sequence Synthetic primer
tcaagtcttagcttcatctcaattatgt

SEQ ID NO: 1174 - DNA, 21 bp, Artificial Sequence
  Description of Artificial Sequence Synthetic primer
tcgatcaccccaaatagaagg

SEQ ID NO: 1175 - DNA, 28 bp, Artificial Sequence
  Description of Artificial Sequence Synthetic primer
tttgagagtgaaattgagaaattgttgg

SEQ ID NO: 1176 - DNA, 30 bp, Artificial Sequence; identical to SEQ ID NO: 166
  Description of Artificial Sequence Synthetic primer

SEQ ID NO: 1177 - DNA, 27 bp, Artificial Sequence
  Description of Artificial Sequence Synthetic primer
gacgctgaaggtaattttaagaaaagc

SEQ ID NO: 1178 - DNA, 21 bp, Artificial Sequence
  Description of Artificial Sequence Synthetic primer
aaggtgagtggttgtgaaagc

SEQ ID NO: 1179 - DNA, 23 bp, Artificial Sequence
  Description of Artificial Sequence Synthetic primer
gcttcacacttgagttatgttgg

SEQ ID NO: 1180 - DNA, 24 bp, Artificial Sequence
  Description of Artificial Sequence Synthetic primer
ctctctatggtctttcttagaggc

SEQ ID NO: 1181 - DNA, 24 bp, Artificial Sequence
  Description of Artificial Sequence Synthetic primer
aggatggtggaactgtatttttgg

SEQ ID NO: 1182 - DNA, 25 bp, Artificial Sequence
  Description of Artificial Sequence Synthetic primer
cctttgaattgctttggtaagaagc

SEQ ID NO: 1183 - DNA, 26 bp, Artificial Sequence
  Description of Artificial Sequence Synthetic primer
gaagaagctcgatggttgaatgaagg

SEQ ID NO: 1184 - DNA, 26 bp, Artificial Sequence
  Description of Artificial Sequence Synthetic primer
agagtggaagctgaaacaatcttagg

SEQ ID NO: 1185 - DNA, 24 bp, Artificial Sequence
  Description of Artificial Sequence Synthetic primer
tgagcaacagagaaatcacatacc

SEQ ID NO: 1186 - DNA, 26 bp, Artificial Sequence
  Description of Artificial Sequence Synthetic primer
gaacacgaagtaagataggaaaaggc

SEQ ID NO: 1187 - DNA, 23 bp, Artificial Sequence
  Description of Artificial Sequence Synthetic primer
gtgccttttcctatcttacttcg

SEQ ID NO: 1188 - DNA, 25 bp, Artificial Sequence
  Description of Artificial Sequence Synthetic primer
caatgctttctttgagcacttttcc

SEQ ID NO: 1189 - DNA, 28 bp, Artificial Sequence
  Description of Artificial Sequence Synthetic primer
tttgagagtgaaattgagaaattgttgg

SEQ ID NO: 1190 - DNA, 30 bp, Artificial Sequence; identical to SEQ ID NO: 166
  Description of Artificial Sequence Synthetic primer

SEQ ID NO: 1191 - DNA, 22 bp, Artificial Sequence
  Description of Artificial Sequence Synthetic primer
gcttgataaccgatgtttcggg

SEQ ID NO: 1192 - DNA, 21 bp, Artificial Sequence
  Description of Artificial Sequence Synthetic primer
gaggtgagtggttgtgaaagc

SEQ ID NO: 1193 - DNA, 21 bp, Artificial Sequence
  Description of Artificial Sequence Synthetic primer
tcacctcaaggctattgtggc

SEQ ID NO: 1194 - DNA, 24 bp, Artificial Sequence
  Description of Artificial Sequence Synthetic primer
agatgtaaaacctagcatgaagcc

SEQ ID NO: 1195 - DNA, 22 bp, Artificial Sequence
  Description of Artificial Sequence Synthetic primer
ggtggaaggagcatgagtttgc

SEQ ID NO: 1196 - DNA, 24 bp, Artificial Sequence
  Description of Artificial Sequence Synthetic primer
ttgaggctaatgcaatgactttgg

SEQ ID NO: 1197 - DNA, 28 bp, Artificial Sequence
  Description of Artificial Sequence Synthetic primer
gggacataaattgtctggataaacttga

SEQ ID NO: 1198 - DNA, 30 bp, Artificial Sequence
  Description of Artificial Sequence Synthetic primer
tttttaagctccttttcaaattcttcataa

SEQ ID NO: 1199 - DNA, 21 bp, Artificial Sequence
  Description of Artificial Sequence Synthetic primer
agctcgatggttgagtgaagg

SEQ ID NO: 1200 - DNA, 22 bp, Artificial Sequence
  Description of Artificial Sequence Synthetic primer
acctagcaagtagagtggaagc

SEQ ID NO: 1201 - DNA, 24 bp, Artificial Sequence
  Description of Artificial Sequence Synthetic primer
tgagcaaaagagaaatcacatacc

SEQ ID NO: 1202 - DNA, 20 bp, Artificial Sequence
  Description of Artificial Sequence Synthetic primer
tttctttccagtgggtgtcc

SEQ ID NO: 1203 - DNA, 23 bp, Artificial Sequence
  Description of Artificial Sequence Synthetic primer
tatggggtatcagagaaagaggc

SEQ ID NO: 1204 - DNA, 26 bp, Artificial Sequence
  Description of Artificial Sequence Synthetic primer
gaacacgaactaagataggaaaaggc

SEQ ID NO: 1205 - DNA, 23 bp, Artificial Sequence
  Description of Artificial Sequence Synthetic primer
gtgccttttcctatcttagttcg

SEQ ID NO: 1206 - DNA, 25 bp, Artificial Sequence
  Description of Artificial Sequence Synthetic primer
caatgctttctttgagcacttttcc

SEQ ID NO: 1207 - DNA, 23 bp, Artificial Sequence
  Description of Artificial Sequence Synthetic primer
tccactcaaatcttagcaacctc

SEQ ID NO: 1208 - DNA, 20 bp, Artificial Sequence
  Description of Artificial Sequence Synthetic primer
tgcaaaaatcggtctcccca

SEQ ID NO: 1209 - DNA, 25 bp, Artificial Sequence
  Description of Artificial Sequence Synthetic primer
tgaagttaattgatgtggtagaacg

SEQ ID NO: 1210 - DNA, 24 bp, Artificial Sequence
  Description of Artificial Sequence Synthetic primer
agaaaccctatatccatgttgtcg

SEQ ID NO: 1211 - DNA, 25 bp, Artificial Sequence
  Description of Artificial Sequence Synthetic primer
caagttgaagaattgaaagaagtgg

SEQ ID NO: 1212 - DNA, 23 bp, Artificial Sequence
  Description of Artificial Sequence Synthetic primer
aaatgataggacaatcccaaacg

SEQ ID NO: 1213 - DNA, 19 bp, Artificial Sequence
  Description of Artificial Sequence Synthetic primer
gcgagtgacaccgttggtt

SEQ ID NO: 1214 - DNA, 24 bp, Artificial Sequence
  Description of Artificial Sequence Synthetic primer
tggatcatcattggggtgttcttt

SEQ ID NO: 1215 - DNA, 23 bp, Artificial Sequence
  Description of Artificial Sequence Synthetic primer
aaagaacaccccaatgatgatcc

SEQ ID NO: 1216 - DNA, 20 bp, Artificial Sequence
  Description of Artificial Sequence Synthetic primer
gggtctttcttaagggcctc

SEQ ID NO: 1217 - DNA, 23 bp, Artificial Sequence
  Description of Artificial Sequence Synthetic primer
tggtggaaggaattagacagtgc

SEQ ID NO: 1218 - DNA, 21 bp, Artificial Sequence
  Description of Artificial Sequence Synthetic primer
atcagcgattgaggaaagtgc

SEQ ID NO: 1219 - DNA, 25 bp, Artificial Sequence
  Description of Artificial Sequence Synthetic primer
gataaactccatccagaatacttgc

SEQ ID NO: 1220 - DNA, 25 bp, Artificial Sequence
  Description of Artificial Sequence Synthetic primer
actttgtaagtttcctccttttcaa

SEQ ID NO: 1221 - DNA, 23 bp, Artificial Sequence
  Description of Artificial Sequence Synthetic primer
gaagctcgatggttgaatgaagg

SEQ ID NO: 1222 - DNA, 21 bp, Artificial Sequence
  Description of Artificial Sequence Synthetic primer
cgcttgccctaacaatcttgg

SEQ ID NO: 1223 - DNA, 24 bp, Artificial Sequence
  Description of Artificial Sequence Synthetic primer
gttgatggcttgctctttagttgg

SEQ ID NO: 1224 - DNA, 20 bp, Artificial Sequence
  Description of Artificial Sequence Synthetic primer
tgtgaccagccacgtcatcc

SEQ ID NO: 1225 - DNA, 25 bp, Artificial Sequence
  Description of Artificial Sequence Synthetic primer
tgtgatgaaatgaataggcgagtgg

SEQ ID NO: 1226 - DNA, 25 bp, Artificial Sequence
  Description of Artificial Sequence Synthetic primer
ccataaccctagcaagattcagagc

SEQ ID NO: 1227 - DNA, 24 bp, Artificial Sequence
  Description of Artificial Sequence Synthetic primer
tcaagttttagcctcatcacaaaa

SEQ ID NO: 1228 - DNA, 25 bp, Artificial Sequence
  Description of Artificial Sequence Synthetic primer
cccaaatagaaggttgataagttgt

SEQ ID NO: 1229 - DNA, 23 bp, Artificial Sequence
  Description of Artificial Sequence Synthetic primer
gcagcgagttgacgaattaaagg

SEQ ID NO: 1230 - DNA, 23 bp, Artificial Sequence
  Description of Artificial Sequence Synthetic primer
tgaaaccataaatccatgttggc

SEQ ID NO: 1231 - DNA, 22 bp, Artificial Sequence
  Description of Artificial Sequence Synthetic primer
tgcttgataactgacattcccg

SEQ ID NO: 1232 - DNA, 21 bp, Artificial Sequence
  Description of Artificial Sequence Synthetic primer
aaggtgagtggtggtgaaagc

SEQ ID NO: 1233 - DNA, 23 bp, Artificial Sequence
  Description of Artificial Sequence Synthetic primer
agtgcttgataactgacattccc

SEQ ID NO: 1234 - DNA, 23 bp, Artificial Sequence
  Description of Artificial Sequence Synthetic primer
atgaaatgtaatgcctagcgtgg

SEQ ID NO: 1235 - DNA, 23 bp, Artificial Sequence
  Description of Artificial Sequence Synthetic primer
tgcaagagataggattgtggagc

SEQ ID NO: 1236 - DNA, 20 bp, Artificial Sequence
  Description of Artificial Sequence Synthetic primer
tctgcaactgaggccaatgc

SEQ ID NO: 1237 - DNA, 25 bp, Artificial Sequence
  Description of Artificial Sequence Synthetic primer
acttaaattgtgcggatcaactacg

SEQ ID NO: 1238 - DNA, 27 bp, Artificial Sequence
  Description of Artificial Sequence Synthetic primer
gtaaactttgtaactttcctcctttcc

SEQ ID NO: 1239 - DNA, 21 bp, Artificial Sequence
  Description of Artificial Sequence Synthetic primer
tcagtgaagctcgatggttgc

SEQ ID NO: 1240 - DNA, 23 bp, Artificial Sequence
  Description of Artificial Sequence Synthetic primer
tgcaatctctcattatcttgggg

SEQ ID NO: 1241 - DNA, 21 bp, Artificial Sequence
  Description of Artificial Sequence Synthetic primer
gagatcattcaccgtctaccg

SEQ ID NO: 1242 - DNA, 24 bp, Artificial Sequence
  Description of Artificial Sequence Synthetic primer
tttttgtctcttttccaacatgcg

SEQ ID NO: 1243 - DNA, 23 bp, Artificial Sequence
  Description of Artificial Sequence Synthetic primer
tgtcgtctcaaatcttagcaacc

SEQ ID NO: 1244 - DNA, 20 bp, Artificial Sequence
  Description of Artificial Sequence Synthetic primer
atgcaaaaatcggtctcccc

SEQ ID NO: 1245 - DNA, 27 bp, Artificial Sequence
  Description of Artificial Sequence Synthetic primer
gccaagttgaagaattgaaagaagtgg

SEQ ID NO: 1246 - DNA, 25 bp, Artificial Sequence
  Description of Artificial Sequence Synthetic primer
actctcaaaatgataggacaatccc

SEQ ID NO: 1247 - DNA, 27 bp, Artificial Sequence
  Description of Artificial Sequence Synthetic primer
agaagtggtaagaaaggagatatttgg

SEQ ID NO: 1248 - DNA, 25 bp, Artificial Sequence
  Description of Artificial Sequence Synthetic primer
aagaaaccctatatccatgttgtcg

SEQ ID NO: 1249 - DNA, 21 bp, Artificial Sequence
  Description of Artificial Sequence Synthetic primer
gaatgtttggcgagtgacacc

SEQ ID NO: 1250 - DNA, 20 bp, Artificial Sequence
  Description of Artificial Sequence Synthetic primer
aagggcctctctagggctcg

SEQ ID NO: 1251 - DNA, 22 bp, Artificial Sequence
  Description of Artificial Sequence Synthetic primer
aagaacaccccaatgatgatcc

SEQ ID NO: 1252 - DNA, 23 bp, Artificial Sequence
  Description of Artificial Sequence Synthetic primer
actaagctcctttttgtgcatgg

SEQ ID NO: 1253 - DNA, 23 bp, Artificial Sequence
  Description of Artificial Sequence Synthetic primer
tggtggaaggaattagacagtgc

SEQ ID NO: 1254 - DNA, 21 bp, Artificial Sequence
  Description of Artificial Sequence Synthetic primer
atcagcgattgaggaaagtgc

SEQ ID NO: 1255 - DNA, 24 bp, Artificial Sequence
  Description of Artificial Sequence Synthetic primer
ttatgaaccccaatactcttttgc

SEQ ID NO: 1256 - DNA, 26 bp, Artificial Sequence
  Description of Artificial Sequence Synthetic primer
aggagcttatgttcttcaaatatacc

SEQ ID NO: 1257 - DNA, 26 bp, Artificial Sequence
  Description of Artificial Sequence Synthetic primer
ggataaactccatccagaatacttgc

SEQ ID NO: 1258 - DNA, 27 bp, Artificial Sequence
  Description of Artificial Sequence Synthetic primer
tcttgttcaaattcctcaaaagattgc

SEQ ID NO: 1259 - DNA, 23 bp, Artificial Sequence
  Description of Artificial Sequence Synthetic primer
gaagctcgatggttgaatgaagg

SEQ ID NO: 1260 - DNA, 21 bp, Artificial Sequence
  Description of Artificial Sequence Synthetic primer
cgcttgccctaacaatcttgg

SEQ ID NO: 1261 - DNA, 22 bp, Artificial Sequence
  Description of Artificial Sequence Synthetic primer
tgatggcttgctctttagttgg

SEQ ID NO: 1262 - DNA, 20 bp, Artificial Sequence
  Description of Artificial Sequence Synthetic primer
agccacgtcatccatgtacc

SEQ ID NO: 1263 - DNA, 28 bp, Artificial Sequence
  Description of Artificial Sequence Synthetic primer
agaatgagcaagagagaaatcatatacc

SEQ ID NO: 1264 - DNA, 20 bp, Artificial Sequence
  Description of Artificial Sequence Synthetic primer
ccatgcaataaccactcgcc

SEQ ID NO: 1265 - DNA, 22 bp, Artificial Sequence
  Description of Artificial Sequence Synthetic primer
taggcgagtggttattgcatgg

SEQ ID NO: 1266 - DNA, 21 bp, Artificial Sequence
  Description of Artificial Sequence Synthetic primer
cctagcaagattcagagcacg

SEQ ID NO: 1267 - DNA, 30 bp, Artificial Sequence
  Description of Artificial Sequence Synthetic primer
aaaatgagaaaatacataaaattgttcgac

SEQ ID NO: 1268 - DNA, 22 bp, Artificial Sequence
  Description of Artificial Sequence Synthetic primer
tcgatctccccaaatagatgga

SEQ ID NO: 1269 - DNA, 30 bp, Artificial Sequence
  Description of Artificial Sequence Synthetic primer
ttgaagaattgaaagaagtagtaagaaagg

SEQ ID NO: 1270 - DNA, 26 bp, Artificial Sequence
  Description of Artificial Sequence Synthetic primer
tcaaaatgataggacaatcccaaacg

SEQ ID NO: 1271 - DNA, 25 bp, Artificial Sequence
  Description of Artificial Sequence Synthetic primer
tgacaagttcaaagatgagaatggc

SEQ ID NO: 1272 - DNA, 20 bp, Artificial Sequence
  Description of Artificial Sequence Synthetic primer
tagtaaatcttccccgacgc

SEQ ID NO: 1273 - DNA, 20 bp, Artificial Sequence
  Description of Artificial Sequence Synthetic primer
acatttgagttgcgtcgggg

SEQ ID NO: 1274 - DNA, 21 bp, Artificial Sequence
  Description of Artificial Sequence Synthetic primer
tagcttggagcctgtttaggg

SEQ ID NO: 1275 - DNA, 23 bp, Artificial Sequence
  Description of Artificial Sequence Synthetic primer
aagaaaaaccctaaacaggctcc

SEQ ID NO: 1276 - DNA, 22 bp, Artificial Sequence
  Description of Artificial Sequence Synthetic primer
ctaagctcctttttgtgcatgg

SEQ ID NO: 1277 - DNA, 25 bp, Artificial Sequence
  Description of Artificial Sequence Synthetic primer
agagacaggagtgtggaactatacc

SEQ ID NO: 1278 - DNA, 24 bp, Artificial Sequence
  Description of Artificial Sequence Synthetic primer
tgtcatcagctattgtggcaaagg

SEQ ID NO: 1279 - DNA, 23 bp, Artificial Sequence
  Description of Artificial Sequence Synthetic primer
tgaagaatttgagcaagggctta

SEQ ID NO: 1280 - DNA, 24 bp, Artificial Sequence
  Description of Artificial Sequence Synthetic primer
gcgtaatgaactctgtaagtttct

SEQ ID NO: 1281 - DNA, 22 bp, Artificial Sequence
  Description of Artificial Sequence Synthetic primer
gtggttaaagaaagctgaacgc

SEQ ID NO: 1282 - DNA, 22 bp, Artificial Sequence
  Description of Artificial Sequence Synthetic primer
gccgacaaatagtagtggatgc

SEQ ID NO: 1283 - DNA, 22 bp, Artificial Sequence
  Description of Artificial Sequence Synthetic primer
tgaataggcgagtggttattgc

SEQ ID NO: 1284 - DNA, 18 bp, Artificial Sequence
  Description of Artificial Sequence Synthetic primer
tgcaagattcagggcacg

SEQ ID NO: 1285 - DNA, 18 bp, Artificial Sequence
  Description of Artificial Sequence Synthetic primer
tcagttgggggaccaatt

SEQ ID NO: 1286 - DNA, 19 bp, Artificial Sequence
  Description of Artificial Sequence Synthetic primer
catccgacgatgttcctag

SEQ ID NO: 1287 - DNA, 47 bp, Artificial Sequence
  Description of Artificial Sequence Synthetic primer
ggatcatcataaccttcttccaagatcttttgcatgcttattttgct

SEQ ID NO: 1288 - DNA, 47 bp, Artificial Sequence
  Description of Artificial Sequence Synthetic primer
tgaaggatccctggaaatatcaaattcttcaagtcgtaaaagtatgg

SEQ ID NO: 1289 - DNA, 22 bp, Artificial Sequence
  Description of Artificial Sequence Synthetic primer
tcttttgcatgcttattttgct

SEQ ID NO: 1290 - DNA, 25 bp, Artificial Sequence
  Description of Artificial Sequence Synthetic primer
ggatcatcataaccttcttccaaga

SEQ ID NO: 1291 - DNA, 22 bp, Artificial Sequence
  Description of Artificial Sequence Synthetic primer
tcttcaagtcgtaaaagtatgg

SEQ ID NO: 1292 - DNA, 25 bp, Artificial Sequence
  Description of Artificial Sequence Synthetic primer
tgaaggatccctggaaatatcaaat

SEQ ID NO: 1293 - DNA, 25 bp, Artificial Sequence
  Description of Artificial Sequence Synthetic primer
ggaagctttttctaagggatttgtg

SEQ ID NO: 1294 - DNA, 25 bp, Artificial Sequence
  Description of Artificial Sequence Synthetic primer
ccttccataaatattcatgaaggat

SEQ ID NO: 1295 - DNA, 18 bp, Artificial Sequence
  Description of Artificial Sequence Synthetic primer
aaatttgatgtgctcgcg

SEQ ID NO: 1296 - DNA, 47 bp, Artificial Sequence
  Description of Artificial Sequence Synthetic primer
tcaagtcgtaaaagtatggatccaacctggaaatatcaaatgatggt

SEQ ID NO: 1297 - DNA, 43 bp, Artificial Sequence
  Description of Artificial Sequence Synthetic primer
ctaggaacatcgtcggatgagacagaaacacctgtatcattca

SEQ ID NO: 1298 - DNA, 22 bp, Artificial Sequence
  Description of Artificial Sequence Synthetic primer
cctggaaatatcaaatgatggt

SEQ ID NO: 1299 - DNA, 25 bp, Artificial Sequence
  Description of Artificial Sequence Synthetic primer
tcaagtcgtaaaagtatggatccaa

SEQ ID NO: 1300 - DNA, 21 bp, Artificial Sequence
  Description of Artificial Sequence Synthetic primer
cagaaacacctgtatcattca

SEQ ID NO: 1301 - DNA, 22 bp, Artificial Sequence
  Description of Artificial Sequence Synthetic primer
ctaggaacatcgtcggatgaga

SEQ ID NO: 1302 - DNA, 25 bp, Artificial Sequence
  Description of Artificial Sequence Synthetic primer
agaggagatgttccgaaatcaattc

SEQ ID NO: 1303 - DNA, 22 bp, Artificial Sequence
  Description of Artificial Sequence Synthetic primer
tcttggaagaaggttatgatga

SEQ ID NO: 1304 - DNA, 18 bp, Artificial Sequence
  Description of Artificial Sequence Synthetic primer
aaatttgatgtgctcgcg

SEQ ID NO: 1305 - DNA, 48 bp, Artificial Sequence
  Description of Artificial Sequence Synthetic primer
tcaagtcgtaaaagtatggatccaataaatattcatgaaggatccctg

SEQ ID NO: 1306 - DNA, 43 bp, Artificial Sequence; identical to SEQ ID NO: 1297
  Description of Artificial Sequence Synthetic primer

SEQ ID NO: 1307 - DNA, 23 bp, Artificial Sequence
  Description of Artificial Sequence Synthetic primer
taaatattcatgaaggatccctg

SEQ ID NO: 1308 - DNA, 25 bp, Artificial Sequence
  Description of Artificial Sequence Synthetic primer
tcaagtcgtaaaagtatggatccaa

SEQ ID NO: 1309 - DNA, 21 bp, Artificial Sequence
  Description of Artificial Sequence Synthetic primer
cagaaacacctgtatcattca

SEQ ID NO: 1310 - DNA, 22 bp, Artificial Sequence
  Description of Artificial Sequence Synthetic primer
ctaggaacatcgtcggatgaga

SEQ ID NO: 1311 - DNA, 25 bp, Artificial Sequence
  Description of Artificial Sequence Synthetic primer
agaggagatgttccgaaatcaattc

SEQ ID NO: 1312 - DNA, 21 bp, Artificial Sequence
  Description of Artificial Sequence Synthetic primer
gggaccaattattcttttgca

SEQ ID NO: 1313 - DNA, 19 bp, Artificial Sequence
  Description of Artificial Sequence Synthetic primer
catccgacgatgttcctag

SEQ ID NO: 1314 - DNA, 48 bp, Artificial Sequence
  Description of Artificial Sequence Synthetic primer
ggatcatcataaccttcttccaagagcttattttgctttcacaaatcc

SEQ ID NO: 1315 - DNA, 47 bp, Artificial Sequence
  Description of Artificial Sequence Synthetic primer
atgaaggatccctggaaatatcaaatcttcaagtcgtaaaagtatgg

SEQ ID NO: 1316 - DNA, 23 bp, Artificial Sequence
  Description of Artificial Sequence Synthetic primer
gcttattttgctttcacaaatcc

SEQ ID NO: 1317 - DNA, 25 bp, Artificial Sequence
  Description of Artificial Sequence Synthetic primer
ggatcatcataaccttcttccaaga

SEQ ID NO: 1318 - DNA, 22 bp, Artificial Sequence
  Description of Artificial Sequence Synthetic primer
tcttcaagtcgtaaaagtatgg

SEQ ID NO: 1319 - DNA, 25 bp, Artificial Sequence
  Description of Artificial Sequence Synthetic primer
atgaaggatccctggaaatatcaaa

SEQ ID NO: 1320 - DNA, 21 bp, Artificial Sequence
  Description of Artificial Sequence Synthetic primer
acaaatcccttagaaaaagct

SEQ ID NO: 1321 - DNA, 22 bp, Artificial Sequence
  Description of Artificial Sequence Synthetic primer
catctcctcttttcatctcatc

SEQ ID NO: 1322 - DNA, 49 bp, Artificial Sequence
  Description of Artificial Sequence Synthetic primer
ttccagggatccttcatgaatatttccataaaattcttggaagaaggtt

SEQ ID NO: 1323 - DNA, 44 bp, Artificial Sequence
  Description of Artificial Sequence Synthetic primer
accctaccatatttcatcttggatccgacgatgttcctaggtca

SEQ ID NO: 1324 - DNA, 24 bp, Artificial Sequence
  Description of Artificial Sequence Synthetic primer
ccataaaattcttggaagaaggtt

SEQ ID NO: 1325 - DNA, 25 bp, Artificial Sequence
  Description of Artificial Sequence Synthetic primer
ttccagggatccttcatgaatattt

SEQ ID NO: 1326 - DNA, 19 bp, Artificial Sequence
  Description of Artificial Sequence Synthetic primer
cgacgatgttcctaggtca

SEQ ID NO: 1327 - DNA, 25 bp, Artificial Sequence
  Description of Artificial Sequence Synthetic primer
accctaccatatttcatcttggatc

SEQ ID NO: 1328 - DNA, 19 bp, Artificial Sequence
  Description of Artificial Sequence Synthetic primer
atgcagtgcatggcttttc

SEQ ID NO: 1329 - DNA, 30 bp, Artificial Sequence
  Description of Artificial Sequence Synthetic primer
ttacatgggaatagggttaataatcaaatc

SEQ ID NO: 1330 - DNA, 20 bp, Artificial Sequence
  Description of Artificial Sequence Synthetic primer
atgtcatatcaagttttagc

SEQ ID NO: 1331 - DNA, 23 bp, Artificial Sequence
  Description of Artificial Sequence Synthetic primer
tgggatttgatctataagtaacg

SEQ ID NO: 1332 - DNA, 23 bp, Artificial Sequence
  Description of Artificial Sequence Synthetic primer
cgggatttgatctataagtaacg

SEQ ID NO: 1333 - DNA, 20 bp, Artificial Sequence
  Description of Artificial Sequence Synthetic primer
atgtctagtcaagtgttagc

SEQ ID NO: 1334 - DNA, 22 bp, Artificial Sequence
  Description of Artificial Sequence Synthetic primer
taatgggatgggatctataagc

SEQ ID NO: 1335 - DNA, 23 bp, Artificial Sequence
  Description of Artificial Sequence Synthetic primer
atgtcaaatattcaagtcttagc

SEQ ID NO: 1336 - DNA, 22 bp, Artificial Sequence
  Description of Artificial Sequence Synthetic primer
taatgggatgggatctataagc

SEQ ID NO: 1337 - DNA, 23 bp, Artificial Sequence
  Description of Artificial Sequence Synthetic primer
actgtggtcgataaccctagttc

SEQ ID NO: 1338 - DNA, 27 bp, Artificial Sequence
  Description of Artificial Sequence Synthetic primer
catgggaatagggttaataatcaaatc

SEQ ID NO: 1339 - DNA, 1677 bp, Artificial Sequence
  Description of Artificial Sequence Synthetic polynucleotide
atgttcacacaaatcttagtatcttcaaatgacaacccaaatataattgttcgtcctacaaaaaaattccacccaaatatttggggagaacgattcttacattacaacatttcacaacaagatttgtattgtcaacaacaaaaggttgaggaattaaaagaagtggttagaagagaaatatttggagattattattatgatgcttggaaaaatcaattgaagataattgatgtggtagaacgtttgggattgtcttatcattttgaaactgaaatagaaaataagcttcaatgcatttacaacaaaactaatattattgatgatgaagatttacaccatgtttctattcgatttagacttctacgacaacatggatttagaatttcttctgatatatttgaaaaattcaaagacgaggatgggaactttaaaaaatgcttggtaagtgatacctttggtttgcttagcttatatgaagcctcacatttgagttgtgttggagaaaatatacttgatgaagcacttgctttcacaacaacacacctaaatgagtttttggtgaagaaaaaagaacaccatgaagacgatgatccattatcgaaagaaatatctcgagccctagagaggcccttaagaaagaccctagtaaatcttcatgcaaggcatttcatttcgatatatgaaaaagatgcctcacataacaaagtattgttacaacttgcaaaattagatttcaatctattacaatgcatgcataaaaaggaacttagtgaagtcagcaggtggtggaaagaatcagattttgtacacaaatttccatttgcaagagataggattgtggaattatacctttggatattaggagtttattatgaaccccaatattgtttggcaagaaatattttagtcaaaatcatagctttggcctcaattgcggatgatatttatgatgcatatggtacacttgaagaacttgagctcctaactcatgcaattgaaaggtgggatataaattgtgtggatacactgaatcaagaatatatgcaaacattttataaggatgttttgaattgttatgaagagtttgagcaagtgctagaaaaagaagaacaatacaaagttcattatgcaaaagcagcgtttaagggattagtccgatctcatttggatgaagctcgatgtttgcatagaggactcatcccaagttttgaagagtacttgaaatttgctttggataattgtggttactacatgttgatactaagctctttggttggaatgaaaactaatattgtaacaaaagatgtttttgagtggctctccaaagatcgaaagattattagagcatctgttactatttgtaggctcatggatgacatagctgaacacaagtttgaggaagatgattatgaggaacattcagcagtggaatgctatatgattgaacatggcgtttgtgaagaggaagcctatgatgaactgaataagctaataatgaatgcatggaaagaaataaatgaagagtttttgaagcctactaaagtagcttcacccatattacttcgtgctcttaacttttcaagggttatggatcttattttcaaatttggagatggttatacacacgttggaaaagtcaccaaagatattgttgctgctttgcttattcatccaatt

SEQ ID NO: 1340 - DNA, 1713 bp, Artificial Sequence
  Description of Artificial Sequence Synthetic polynucleotide
tcaagtacttgctaccccatcaaatgttctgtggtccataaccctagttctactactattgttagaagatcaggaaactatgaacctcccatttggtcttttgattatattcattctctttcaagcaaatataagggagaagcttatacaaatcgactgaaaaagcttgagaaagatgtgaaaatgatgctacttggcatggaaattaattctttagcccaacttgagttcattgatacattacaaaggcttggaatatcttatcattttaaaaatgaaataaatactattttaaagaaaaaatacactaataatacaaatattatgaataaaaaccctaactatgacttatatgccacttctttggaatttaggcttctacgggagtatggatttgtagtacctcaagaaattttcaatgattttaaggatgatgaaacaggagagttcaaggcaactataaatgatgatattatgggagcattggctttatatgaagcttcattttatgggaaaaatggtgaaagtgttttggaagaagcaagaattttcacaaccaaatgtcttgaaaaatacaaaatattgtcaagtgataataataataataataataatatattattattagtggatcatgctttggaaatgccacttcattggagaatcacaaaatcagaagctagatggttcattgaagaaatatatgagaaaaaacaagacatgatgatgaattctactttgcttgagtttgccaaattggatttcaacattgtgcaatcaacatatcaagaagagctcaaatatctctctaggtggtggaagcattctaaacttggagagaaattgcctttcgttagagatagattgatggagtgtttcatgtggcaagttggaataagatttgagccagaattcagttactttagaataattgctacacaaatctatgttctaatcacaataattgatgatatgcatgacatatatgcaactttggaggaactacaactcttcgctaatgctcttcaaaggtgggatttgaaagtaatagatgagttaccggattacatgaagatggctttcttcattttatacaatttcacaaatgaaatggtgtttaatgtattaggagaacatggttttgttaaccttgaatacttcaagaaattgtggatagacatatgtaaacatcaattgcaagaggcaaaatggttctatagtggataccaaccaacattgcaagaatatactgaaaatgcttggatttcagtaggaggaccagcttttcttatgcatgcttatttctgttttacaaatctcataacaaatgaagaagtggaatgtcttaaagagggttatcccaatattgttcgacatggatccctaatattacgacttgcagatgatctaggaacatgttcggatgaaatgaaaagaggcgatgttcctacatcaattcaatgttacatgcatgatactggtgtttctgaagacgaagctcgtgagcacatcaagcatttgataagcgaatcatggaaggagatgaataatgaaaatggagatcataattcttctttctcgaaagaatttgttcatgtttgcaaaaatcttggtagagcgtcacaatttatataccaatatggagatggacatgcttctcaaaatactctatcaaaagatcgtatttttaaaattattattaatcctttcccc

SEQ ID NO: 1341 - DNA, 1572 bp, Artificial Sequence
  Description of Artificial Sequence Synthetic polynucleotide
caacaacaaaaggttgaggaattaaaagaggtgattagaagagaaatatttggggaatcatcaatatcatcatcagcttattatgatgcttggaaaaatcaattgaagataattgatgtggtagaacgtttgggattgtcttatcattttgaaactgaaatagaaaatatgatagaacaaatctacaacaaaaccaataattttgatcttcttaaggatgaagagttgcatgatgtttccttgtgggttagacttctacgacaacatggatttagggtttcttctgatatatttaaaaaattcaaagatgaggatggaaactttaaaaaatgtttggtaagtgatactcttggtttgcttagcttatatgaagcctcacatttgagttgtgttggagaaaatatacttgatgaagcacttgctttcacaacaacacacctaaatgagtttttggcgaagaaaaaagaacaccatgatgacgatgatccattatcgaaagaaatatatcgtgccctagagaggcccttaagaaagaccctagtgaatcttcatactaagcatttcatttcgatatatgaaaaagatgcctcacataacaaagtattgctacaacttgcaaagttagattttaatctattacaatccctacacaaaaaggagcttagtgaaatcagcaggtggtggaaagaatcaaactttgtacaaaaattcccttttgcaagagatagaattgtggagctctatctgtggatgttaggagtctattatgaaccccaatactctttggcaagaaatattttagccaaaatcatagctttttcctcaattgctgatgatatttatgatgcatatggtacatttgaagagcttgaactcctaactcaagcaattgaaaggtgggatataaattgtattgatacactcaatcaagaatatttgaaaacattttataaggatcttttgaattgttatgaagagtttgagcaagtgcttacaaaagaagaaacttatagagttcattatgcaaaagaagtgtttaaagagttaatccgatcatattttgatgaagctcgatggttgaatagtggacgtgtcccaaattttgaagagtacatggaatttgcaaccattaattgtggttactatatgttgatagtgagttctttggttggaataaaaactagtattgtaaccaaagatgtttttgagtggctctccaaagatcgaaagattattagagcatctgttattatttgtaggctcatggatgacatagctgaacacaagtttgagaaagacaaatatgatgaagattctgccattgaatcgtatatgatgcaacatggtgtttgtgaagaagaagcctatgatgaactcaataagcttataattaattcatggaaagaaataaatgaagagtttttgaagccaactaaattagcttcacccatattacttagggttcttaatttttctagggttatggatcttctctacaaaaatggtgatggttttacacatgttggaaaagtcaccaaagatactgttgctgctttgctaattgatcccatt

SEQ ID NO: 1342 - DNA, 1584 bp, Artificial Sequence
  Description of Artificial Sequence Synthetic polynucleotide
aataacaaagagagttatgtgaagattattgagctgaaggaacaagtgaagaataaattacttcatggtcttcatcctttggaaaaccctttagagacacttgaatatattgatgatattcaacgattggggttatcttattatttcgaaaatgaaattgaacaagttttgaagcaatttcataataataacaatcttcatcacgattttggtgataacctttatgctgatgctcttcgctttcggttgcttagggaacaaggttataatagtgcatgtgaggtattcgccaaatataagaatgagagaggaaaatttaaggaatcaattttgagtgacattcgaggaatgttgaacttgtatgaagcttcacaaatgagagtttgcagagaaaaaatactagatgaagcacttatcttcacaactactcatcttcagtccttagtggaaacatcccaattgagctctccttatttagacctagtgaaacatgccctaatgcaccctattcgaaagagcttacaaagaagagaggcaagactttacatatcacgttaccaccaactaccttctcacgaaaagcttcttttaactcttgccaaactagatttcaacttgcttcaacaactacatcaaaaagaattaaattatattacgaggtggtggaaggagtttgacttcaaaagtaagcaatctttttcaagagacagaatagtggagtgttacatttggaattatggagtatattttgaagctcaaacttcccaaattaggttgatgatgaccaagttgatttctttgcttactattattgatgattcttatgatagctatggtacacttgaagagctaaggccttttactgaagcagttagaaaggtatttaattatatatacaaacatatgaagtgggatatatctgctactgaaaatttactagagtacatgaaagtatgttacaagaaatttttagaatttttcaatgaaattgaagaatttacaaaggaaaatccatactgtgcgagctatgttaagaaagggttgcaatgtatggttagagcttatagtaaggaagcccaatggttacacaataaatatatgccaacatttgatgaatattatccaattggacttgataatgctggatctgatgagctaatatcaatggccttttgtggaatgggaaatgttgtaacaaaagaatctatggattggatattttctcaaccccaacctaagattatacgaactatgtcaattgttggaagacttatgaatgacattggctaccacaagtcggatcgaagaaaattatcaaaagatgtagtggcttccgctgtggagtgttacatcaaacaatatggtgtaacagatgaagaagctatagagaaacttaacgaacaagttaatgattcatggaaggatctcaatgaagatcttctttatccaatcgctattcctagaccactcttaatgagagttctcaatcttgtacgagtgaaccatgagatgtatagagaaggcgacggttttacacaaccaacattgctcaagaatttgattgatactctcatcatcaatccc

SEQ ID NO: 1343 - DNA, 1689 bp, Artificial Sequence
  Description of Artificial Sequence Synthetic polynucleotide
agtactacaatattcaaagaacaaccggaagcaattgtccgacgatcaggaaactacaaacccacgttgtgggatgctcattttttcaaatcactccaagtcatatatacggaggaaagctatgggaaaagaatcaatgagctgaaagaagatgtgagaagaattcttgagaaggaggctgagaatcctttagttaaacttgaacagatcaatgatttgagcagacttggaatatcttatcactttgaagatcaaattaagacaatattaaacctgacatttaacaataataatgcattgtggaagaaagataatctgtatgccactgctcttcatttcaaactccttagacaatatggattcaatcctgtatcttcagaggttttcaatgccttcaaagaccagaaaaaagagttcaaagaaagtttgagtaaggatgtgaaaggaatggtatgtctatatgaggcttcattctattcatttaggggtgaacccatattagatggagcaagagacttcacaaccaaacatctgaaacaatacttgatgacgaggcaatgtcaaactaaatctgttgaccatgatgatgatgatgatcttgtgaaacttgtggagcatgccttggatcttccactccattggagattgccaagattggaggctaggtggttcattgatatgtatgctgaaaggaattatgatatgaatccaacttttcttgacttcgctaaacttgactataacttcgttcagtcagcataccaaaaagagctcaaatatatttcaaggtggtggagtggttctaggttgacagaaaggctaccatttgctagagacagagtagtggaaattttctattctgcagtggcattaaaatatgaggcagaatttggatttgttagaactgtgatgacaaaaattggtctcttgctaacactcatggacgatatatatgatgtgtatggtacactagatgagcttcagctttttctaggggcaattgagaggtggaatataaatgaattggatcagctacccgattacatgaagatattgtttgttgctttttataacaatgtgaatgagatatcttattatgtccttaaagaaaatgggattcacatcatcaaatatctgaagaaagcgttgggggatctgtgtaaatgctatatggaggaggcaaaatggtttcacagtggacacataccaagtttagaagaattcattgagaatggatggaaatcaattacaatacctctttgtcttatctatcattattgcttaattacaacttcaattacagaacaagacatggaacacttgcttcaatatccaactattttacgtgtctcaggaactgtctttcgatttatagatgatttgggaacctcatcagatgaacttgaaagaggagataatccttcatcaattcaatgctacatgcgtgaaaagggtgttagtgaaaatgaaagccgtgaacacatatggaatttaataagtgaaggatggaaggaaattaatgaagtcaaagcttcaaactctccatactctcaagtgtttattgaatctgccattgattttgttagaggagcaatggagatgtatcacaagggtgatggctttggtactaatcaagaccgatatcttaagaccaaagttgtcaacatgttcttcgatccaattccaatt

SEQ ID NO: 1344 - DNA, 1683 bp, Artificial Sequence
  Description of Artificial Sequence Synthetic polynucleotide
caatgtagtttggccaaaagccctagtagtgatactagtacaattgttagaagatcagccaactatgatcctcccatttggtcttttgatttcattcagtctcttccatgcaaatataagggagaaccctatacaagtcgatcgaataagctaaaagaagaagtgaaaaagatgttagttggaatggaaaactctttagtccaacttgagttgattgatacattacaaagacttggaatatcttatcattttgagaatgaaatcatttctattttgaaggaatatttcactaatattagtactaataaaaaccctaaatatgatttatatgccactgctttcgaatttaggcttttacgcgaatatggatatgcagtgcctcaagaaatttttaatgattttaaggacgagacgggaaagttcaaagcgagtattaaaaatgatgatattaagggagtattggctttatatgaagcttcattctatgtgaaaaatggcgaaaatattttggaggaagctagggttttcacaacagaatatctcaaaagatatgtaatgatgattgatcaaaacataatattaaatgataatatggcaatattagtgagacatgccttggagatgccacttcattggaggactataagagcagaagctaagtggttcattgaagaatatgagaaaacacaagacaagaatggcactttgcttgaatttgcgaaattggatttcaacatgcttcaatcaatatttcaagaagatctaaaacatgtctcgaggtggtgggaacattctaagcttggaaagaataaaatggtttatgctagagatagattggtagaggcttttctatggcaggttggaataagatttgagccacaattcagccactttaggagaatatctgcaagaatatatgctctaattacaatcatagatgacatatatgatgtgtatggaacattggaagagttagagcttttcaccaaggctgttgagagatgggatgcgaagaccatacacgagttaccagattatatgaagttgcctttctttactttatttaacaccgtaaatgaaatggcgtatgatgtattagaagagcataattttgtcaccgttgaatacctcaagaactcgtgggcagagttatgtaggtgctatttggaagaggcaaaatggttttatagcggatacaaaccaaccttgaagaaatatattgagaacgcctcgctttcaataggaggacaagttatttttgtgtatgcttttttctctcttacaaagtccataacaaatgaggccttagagtccttgcaagagggtcattacgctgcatgtcgccaaggatccttgatgttacgacttgcagatgatcttggaacatcatcagatgaattgaaaagaggtgacattcctaaatcggttcaatgttacatgcacgagactggtgtttctgaagatgaagctcgtgaacacatcaaatttttgataagtgaaatatggaaggagatgaatgatgaagatgaatataactctattttctctaaagagtttgttcaagcttgcaaaaatcttggtaggatgtcattatttatgtatcaacatggagatggacatgcttctcaagatagccattcaaggaaacgtatttcagatttaattattaaccctattccttta

SEQ ID NO: 1345 - DNA, 1680 bp, Artificial Sequence
  Description of Artificial Sequence Synthetic polynucleotide
gatagttctgttactaatattgttcgaagatcagccaactttgaaccatccatttggtcttttgatttcattcagtctctttcaagcaaatataagggagaaccttatacaagtcgagtaaaaaaactcgaggaagatgtgaaaaggatgctagttgagatggaaaactctttagctcaacttgaactcattgacacgttacaaagacttggagtatcttatcgttttgagaatgaaataaatactattttaaaagaaaaatatgtcaatattaatggtaatattaataaccctaattataatttatatgccactgctcttgaatttaggcttctacgtcaacatggttatgctgtacctcaagagacttttaattattttaaggacgagacaggaaaattcaaaacaaacataagtggtgatatcatgggagtattggccttatatgaagcttcattctatgagaaaaaaggcgaaagcattttagaggaagctagaattttcacaactgaatgtctcaaaaactacacaataatgatatcagagcaaaagaaattgatgattaataataattatgattattattacgatattgaagtagtgaatcatgctttggagctcccacttcatcgaaggaccacaagaatagaagctaagtggttcatcgatacgtataagaaaaaacaagacatgaatcctattttgcttgagtttgccaaactcgatttcaacatgatacaatcaacacatcatgaagatttgaaacatatattcaggtggtggaggcatactaaacttggtgagaaattgaattttgcaagagatcgattgatggaatgttttttatggaaagttggaataagatttgagccaaaattcagttattttagaacaacaactgtcaaattacttgagctaataacattaattgatgatatatatgatgtttatggaacattagatgaattagagcttttcaccaaagctattgagagatgggatgtggaaatgataaatgaattaccagaatacatgaagatgccttatattgttttacacaatacaataaatgaaatggtatttgagatattaagagatcaacacatcaccatcaaaattcaatacttaaagaaaacgtgggtagatctgtgtagatgttttctacaagaagcaaaatggtattacagtggatacacaccaacattggaagaatatattgaaaatggttggatttcagtgggagcaccagttcttattgtgcatgcttattttcattcaaataataataaagagattttcgaatgcttggaacatggttattatcctaccataattcgtcacagtgccataataatacgacttacaaatgacctagcaacatcatctgaagaactgaaaagaggtgatgctccgacgtcaattcaatgttacatgcaagaaaaaaatatatgtgaagaggaagctcgtgaacatattaagtttttaataagtgaagcgtggaaggagatgaataacagtgaatctgatgatggattaatatatccaatctcattgattgaagatgcgagaaactttgctaggataggattatacatgtatcaacatggtgatggtcacagttctcaggataatctatccaaagaaagaatttcatcctttattattaaacctattccccta

SEQ ID NO: 1346 - DNA, 1590 bp, Artificial Sequence
  Description of Artificial Sequence Synthetic polynucleotide
aataacaaagagagttatgtgaagattattgagctgaaggaacaagtgaagaagaaattacttcatggtcttcaacctttggaaaatcctttagagacacttgaatatattgatgatattcaacgattggggttatcttattattttgaaaatgaaattgaacaagttttgaagcaatttcataataataataacaatcttcatcatgattttggtgataataacctttatgctgatgctcttcgctttcggttgcttagggaacaaggctattttatagcatgtgaggtatttgccaaatataagaatgagaaaggaaaatttaaggaatcaatttcgagtgacattcgaggaatgttgaacttgtatgaagcttcacaaatgagagttcgcagagaagaaatactagatgaagctcttatcttcacaactactcatcttcagtccttagtggaaacatctcaattgagctctccatatctagacctagtgaaacatgccctaatgcaccctatccgaaagagctttcaaagaagagaggcaagactttacatatcactttaccgtaaactaccttctcacgaagagcttcttttaaccctcgccaaactagatttcaacttgcttcaacaactacatcaaaaggaattgaattatattacgaggtggtggaaggagtttgacttcaaaagtaagcaatctttttcaagagacagaatagtggagtgttacatttggaattatggagtatattttgaagctcaaacttcccaaattaggttgatgatgaccaagttgatttctttgcttactattattgatgattcttatgatagctatggtacacttgaagagctaaggccttttactgaagcatatatatatttacttaattaatatgatatgatcatcaggtgggatatttttgctactgacaacttgccagagtacatgaaagtatgttacaagaaatgtttagaatttttcaatgaaattgaagaatttacaaaggaaaattcattctgcgcgagttatgttaagaaagggttgcaatgtatggttagagcttatagtaaggaagtccaatggttacacaataaatatatgccaacatttgatgaatattatccaattggacttgataatgctggatctgaagagctaatatcaatggccttttgtggaatgggagatgttgtaacaaaagaatctatggattggatattttctcagccccaacctaacattatacgaactatgtcaattgttggaagacttatgaatgacattggctaccacaagtcggatcgaagaaaattatcaaaagatgtagtggcttctgctgtggagtgttacatcaaacaatatggtgtaacatatgaagaagctatagagaaacttaacgaacaagttaatgattcatggaaggatctcaatgaagatcttctctatccaatcgctattcctagaccactcttaatgagagttctcaatcttgtacgagtgaaccatgagatgtacagagaaggtgacggttttacacaaccaacattgctcaagaatttgattgatactctcatcatcaatccc

SEQ ID NO: 1347 - DNA, 1896 bp, Artificial Sequence
  Description of Artificial Sequence Synthetic polynucleotide
atggctgcattagtgtccactgtaagtagtattattaggtggaatagtaataataataataccttcactagatctgtgaaatcatgtttaagttcaaattaccataataataataatattattcataataaaactgcattaatgtccaccaataataataataataataataataataataatcagaagagatcagcaaactatcaacccccactttggcattttgactatgttcaatcactttctactcctttcaaggaaggagcatatgccaaaagagttgagaaagtaaaagaagaggtaagagtaatggtgaagagagcaaaagaggaggagaagcctttatctcaacttgagctaattgatgtaatgcaaagacttggaatctcttaccactttgagaatgaaattaatgacatattgaaagatatatataacaacaataatgtgtgcaacaccaacaaaaatgtgtatgccaattctcttgaatttagactcctaagacaacatggttatccagtgtctcaagaaatttttagtacgtgcaaagatgaaagaggcaattttatggtgtcttccaatgatatcaaaggaatgttatctttatatgaagcttcattctatttggtagaaaatgaagatggtattttggaagagacaagacacaaaacaaagaaatatcttgaggaatacataatcatgatcatggaaaaacaacaatcattattagatcaaaataataataataatgataatgattatgattatgaactagtgagccatgcattggagcttccacttcattggagaatgttaagattggagagtaggtggtttattgatgtgtatgagaagagactagacatgaaccctactctacttaccttagctaaactagatttcaacattgtccaatcaacataccaagatgatcttaaatatgttttcaggtggtggaaaaacactggtttgggagaaaagttgaaatttgcaagagatagaatgatggtgaattttttatggacaataggagtttcatttgagccacactacaaaagtttaagaagaatggctacaaaagtaaatgctttaataacaataatagatgacatatatgatgtttatggtacacttgatgaattggagctcttcactaatgtggttgagagatgggatgttagtgctatgaatgggctccctgagtatatgaagtcatgtttccttgctttgcacaattttataaatgactttgcatttgatgtgttaaaaggaaaaagcctccatgtaaaaaaataccttcaaaaaacgtgggcagatctttgtaaatcttatttaagagaagcaagatggtattatgatggatacacaccaagatttgaagagtacattgagaatgcatggatatcaatatcaggacctgttatactatcacatttatacttttttgtagtaaatccaaacgaggaagatgccttattaagtacttgctttaatggataccctaccataatacgccattcgtcgatgattttacgacttacaaatgatctcgccacttcaacggatgagttgaaaagaggtgatgttcccaaatcaattcaatgcaaaatgcatgaagatcgtgtatgtgaagagaaagctcgtcaacatattaagttattaataaatgaaacatggaagtttcttaataaagattgcataaatttggatgatgattactctccaatgttctctaaatctaataataatattaataaggctttcattgaaatgtgtttaaaccttggtagaatgtcacattgcatttatcaatatggagatggacatggaattcaagatcgccaaacaaaagatcatgtactatcattacttattcaccctattcttcttacccaa

SEQ ID NO: 1348 - DNA, 1491 bp, Artificial Sequence
  Description of Artificial Sequence Synthetic polynucleotide
gaacgctccatagaagctttgaacttggtcaacgctgtgcttagagtcggcattgattatcatgtccaagatgagattaaatctatccttgaaagggaacatataattttcagtgaccatattagtaaccaatattttaataatattaatcaagaccacctgtatgaggtttctcttcgtttccgattactgagacaaggaggttatgatgtctctccagatgtgtttaacgagttgatgaaggacaaaaagggaaatttcaatgttttagtggaagaagacagagagggactaagagaattgtttgaagcctcacaagtaagaatagaaggagaagaggtactagaggaagctgaagtgttcagtggcgaacacctaaaagaatgggcaaatcttcatcgtcatacgagtgaagctcgaagtattcaattaacgttagaccaaccttgtcataaaagcttggccagggtcacatctccaaactttttggacagtttctcagccaacactgctaccagagaccaaggctggacctggatgacccttttaaacaaactggtcacaatggacttcaaaatagttcagtctattcaccaaagggaaattgttcaagtctccaaatggtggaaagagcttggtttggcagaggagttgaaatttgctagagaccaacccctaaaatggtacctctggacagtggcaagtctaccagatccaagcttgtctgaagagaggattgaactaactaaacccatttctctcgtctacataatagatgatatttttgatgtttatgggacactagacgaactcactcttttcacagacgcagttaataattgggaaattaaagagcagttacccgactatttgaagatatgtttcaaggcacttgatgatataaccaacaaaattagctacgtggtgtacagaaaacatggatggaacccaattgactccttaagaaaatcgtggggaaaactgtgcaatgcgttcttgttagaagcagagtggtttggttgtgggaagttgccaaatgaagaggagtatttgaagaacgcgattgtgagttctggtgtacacgtggtgctagttcatatgttctttctcttaggtgaaggtatatccatggaagctgtaaacttactggacaacattccaggccttgtgtcttctacagcagcaattcttcgtctttgggatgacttaggaagtgccaaggatgagaatcaaaatgggcatgatgggtcttacgttgagtgctacatgaaaagacacaaggagtgttcaatgggcgaagcaagggagcaagtgattcgtatgattaaaaatgagtgggaacgcctcaataaggagtgtttctcgccaaaacattttcccatgtgcttcagaaagggttgtcttaatgctgctaggatggttccagtgatgtatgactatgatgatcatcatcgccttccaagcctccagaattatatcaactcacttctc

SEQ ID NO: 1349 - DNA, 1677 bp, Artificial Sequence
  Description of Artificial Sequence Synthetic polynucleotide
gatagttctgttactaatattgttcgaagatcagccaactttgaaccatccatttggtcttttgatttcgttcagtctctttcaagcaaatataagggagaaccttatacaagtcgagtaaaaaaactcgacgaagatgtgaaaaggatgctagttgagatggaaaactctttagctcaacttgaactcattgacacgttacaaagacttggagtatcttatcgttttgagaatgaaataaatactattttaaaagaaaaatatgtcaatattaatggtaatattaataaccctaattataatttatatgccactgctcttgaatttaggcttctacgtcaacatggttatgctgtacctcaagagacttttaattattttaaggacgagacaggaaaattcaaaacaaacataagtggtgatatcattggagtattggccttatatgaagcttcattctatgaaaaaaaaggcgaaaacattttagaggaagctagaattttcacaactgaacgtctcaaaaactacataatgatatcagagcaaaagaaattgatgattaataataattatgattattattacgatattgaagtagtgaatcatgctttggagctcccacttcatcgaaggaccacaagaatagaagctaagtggttcatcgatatgtacaagaaaaaacaagacatgaatcctattttgcttgagtttgccaaactcgatttcaacatgatacaatcaacacatcatgaagatttgaaacatatattcaggtggtggaggcatactaaacttggtgagaaattgaattttgcaagagatcgattgatggaatgttttttatggaaagttggaataagatttgagccaaaattcagttattttagaacaactgtcaaattacttgagctaataacattaattgatgatatatatgatgtttatggaacattagatgaattagagcttttcaccaaagctattgagagatgggatgtggaaatgataaatgaattaccagaatacatgaaaatgccttatattgttttacacaatacaataaatgaaatggtatttgagatattaagagatcaacaaatcaccatcaaaattcaatacttaaagaaaacgtgggtagatatgtgtagatgttttctacaagaagcaaaatggtactacagtggatacacaccaacattggaagaatatattgaaaatggttggatttcagtgggagcaccagttcttattgtgcatgcttatttttctcattcaaataataataaagagattttcgaatgcttggaacatggttattatcctaccataattcgtcacagtgccataataatacgacttacaaatgacctagcaacatcatctgaggaactgaaaagaggtgatgctccgacgtcaattcaatgttacatgcaagaaaaaaatatatgtgaagaggaagctcgtgaacatattaagtttttaataagtgaagcgtggaaggagatgaataacagtgaatctgatgatggattaatatatccaatctcattgattgaagatgcgagaaactttgctaggataggattattcatgtatcaacatggtgatggtcacagttctcaggataatctatccaaagaaagaatttcatcctttattattaaacctattccccta

SEQ ID NO: 1350 - DNA, 1656 bp, Artificial Sequence
  Description of Artificial Sequence Synthetic polynucleotide
atgtcaaatattcaagtcttagcttcatctcaattaagtgacaaaattattgctcgtccaacaacaaattttcacccttctatttggggcgatcgattcctccattacaatatttcagaacaagatttggtttgcaaagaagaaagaattgaagaattaatacaagttgtaaagaaagagatattatcttcaaatcatgatcaattgaagttgattgacaatctccaacgtttgggattatcacatcattttgagagtgaaattgagaaattgttggaacaattaaatatcggcactcatcatcaaaatcatcaagatctacatgatgcttctctttggtttagattattaagacaacatggatttaatgtttcatcaagtatatttgaaaaatttaaagacgagaaaggtaactttaaagaaagcctaataactgatgttccaggtttgcttagcttgtatgaggcttcccacttgagttatgttggagaaagcatactagatgaagcacttgctttcacaactactcaccttaaggctattgtggcaaatagtaaagatcatccattatcacatcaaatatccatagccttgcataggcctctaagaaagaccatagagaggcttcatgctaggttttacatctcaatctatgaaaaggatgcctctcataacaaagtattgctagagcttgcaaagttagatttcaatctacttcaatgtttccacaaaaaggaacttagtgaaattatgaggtggtggaaagagcatgagtttgcaaagaaatttccttttgcaagagataggatggtggaacagtatttttggatattgggtgtatattatgaacccaaatactctcgagcaagaaaacttctaaccaaagtcattgcattagcctcaatcactgatgatatttatgatgcatatggtactattgatgagcttgagcttcttacaaaagcaatgcaaaggtgggacataacttgtatggataaacttgagccagaatatttaaagacatattataaggcaatgttggattcttatgaagaatttgaaaaggagcttaaaaaggaagaattatacaaacttgagtatgcaaaagaagagatgaaaagaattattggagcttattttgaagaagctcgatggttgagtgaaggatatttgccaaggttggatgagcatttgagagtttcatatgtttcttgtggttacgttttgttgatagccacaagttatgtaggaatgcatgatattgtaacacatgaaactctaaattggctctccaaagaccctaaaattgtttcagcttccactctccttgctaggttcatggatgatattggttctcgcaagtttgagcaaaagagaaatcacataccatctacagtggattgttacatgaaacaatatggggtatcagaggaagaggcaattaaagaacttaataaaagagtggacacccactggaaagaaattaatgaagactttattaggccaactgttgtgccttttcctatcttagttcgtgttcttaattttacaaaaacagtagatcttctttacaaagagggcgatgatcaatacacaaatgttggaaaagtgctcaaagaaagcattgctgctttgcttatagattcaatcccatta

SEQ ID NO: 1351 - DNA, 1896 bp, Artificial Sequence
  Description of Artificial Sequence Synthetic polynucleotide
atggcttttcaccaatttgctccatcatcatccctccctatttggagtagaattaataatcgttttacaccaaaaacttctattattacttctatttcaaaaccaaaatcaaacttgaaatcgagatcgagatcaagtacttgctaccccatacaatgtactgtggtcgataaccctagttctacgattactaataatagtgatcgaagatcagccaactatggacctcccatttggtcttttgattttattcaatctcttccaatccaatataagggagaatcctatacaaggcaattaaataagttgaagaaagaagtgacaagaatggtccttggactggaaattaactctttagccctacttgagctaattgatacattgcaaaggcttggaatatcttaccattttaagaatgaaataaacactattttgaagaaaaaatacactgataattatattaacaataatattattattactaaccctaattataataatttatatgccattgctctcgaatttaggcttctacgccaacatggctatacagtacctcaagaaatttttaatgctttcaaggataagagagggaaatttaaaacatgcttgagtgatgatattatgggagtattgtgtttatatgaagcttcattctatgctatgaaacatgaaaatattttggaggaagcaagaattttttcaactaaatgtcttaaaaaatacatggagaaaatggagaatgaggaagagaaaaaaatattattattagatgataataatattaatagtaatttgctattaattaatcacgctttcgagcttccgcttcattggagaataacaagatcggaagctaggtggttcattgacgaaatatatgagaaaaaacaagacatgaattcgactttgttcgagtttgcgaaattagattttaatatagtgcaatcaacacaccaagaagatttacaacatttatcgaggtggtggagggattgtaaacttggtgggaaattgaattttgctagagatagattgatggaagctttcttatgggatgttggactaaaattcgagggagaattcagctattttagaagaataaatgcaagattatttgtgcttataacaataattgatgatatttatgatgtatatggaactttggaggaattagagcttttcactagtgctgttgaaagatgggatgtgaaattaatcaatgagttaccagattacatgaagatgcctttctttgttttacacaataccataaatgagatggggttcgatgtattagtacaacaaaactttgtcaacattgaataccttaagaaatcgtgggtagatttatgtaaatgttatttacaagaagcaaaatggtattatagtggataccaaccaacattggaagaatacactgagttgggttggctttcaataggagcatcagtaattcttatgcatgcttatttttgtttcacaaatcctataacaaaacaagacttgaaaagtttgcaattgcaacatcattatcccaacataattaaacaagcatgcttgattacaaggcttgcagatgatttaggaacatcttcggatgaattgaatagaggcgacgttcctaaatcgattcaatgttacatgtacgataataatgctaccgaagacgaagctcgtgaacacatcaagttcttgataagtgaaacatggaaggatatgaataagaaagatgaagatgagagttgtttatcagaaaattttgttgaagtttgcaaaaatatggctagaacagcactattcatatatgagaatggagatggacatggttctcagaatagtttatcaaaagaacgtatttcaaccttgattattactccaattaatattcctaaa

SEQ ID NO: 1352 - DNA, 1737 bp, Artificial Sequence
  Description of Artificial Sequence Synthetic polynucleotide
tcaaaatcaaaatcaagtacttgctaccccatccaatgtactgtggtcaataatagtagccctagctctactactaaattttctattgttcgaagatcggcaaattatgaacctccgatttggtctttcgattacattcaatctctttcaacccaatataagggagaatcctatacaaggcaattaaataagttaaagaaagaagtgaaaaggatgctccttcgaatggaagttaactctttagccctactcgagttaatcgatacattgcaaaggcttggaatatcttatcattttaagaatgaaataaacactattttgaagaaaaaatacacaaatacttatattaacaataatattactaaccataattataatttataccccattgctctcgaatttaggcttctacgccaacatggctatgcagttcctcaagaaatttttaatattttcaaagacgtgagtggaaaatttaaagcaatcataaatgacgatgatattatgggaatattggcgttatatgaagcttcattctatgagaaaaatggtgaaagcatattggaggaagctagggttttcacaaccaagtgtctcgaaaattacatgatgataacggaggaaaacaaattagatgatgatgatgatcgtaataatattattgtagtaattagacatgcgttagagcttccacttcattggaggactgcaagaacggaggccaagtggtttatcgatgtatacgagaaaagacaagatatgaattctactttgcttgagtttgcaaaactagatttcaacatggtacaatcaatgcatcaagacgatctaaaacatttgtctaggtggtggagtcatactaaactcggagaaaaactgaattttgtgagagataggttgatggggtgttttttatggaatgttggattaagatttgagccagatttcagctactttaggagaatatctgcaagaatatatgttcttattacaataatcgatgatatatatgatgtttacggaacattagaagaattagagcttttcactcgtgttgttgagagatgggatgtaaaactactaaaagacttaccagattatatgaagatgccttttcttgttttacacaataccattaatgaaatggcattagatgtattaaaagaaaaacactttgtcaacattgaatgcctcaagaaaacgtgggttgatatgtgtagatcttttctacaagaggcaaaatggtactataatggatacacaccaacatttcaagaatacattgaaaatggttggatttcagtaggagcaccagttattcttgtgcatgcttatttctcttttgcaaatcccataactaaagaggttttggaaatcttccaacaaggttatcccacaataattcgtcaatctgccatgatcgtacgtttggcaaatgatctagcaacatcatctgaggaattcgaaagaggtgactcgcctaaatcaattcaatgttacatgcaagaccataatgtatctaaagaggaagctcatgaatacattaagttttcaataagagaaacatggaaggagatgaatagtacttttaatattgaagatggaaattcatcattggttgatgatgccaaaaactttgcaagaatgggattatttatgtatcaacatggagatggacatagttctcaagataatttagcaaaagagcgtatttcgaaattcattatcaatcctata

SEQ ID NO: 1353 - DNA, 1452 bp, Artificial Sequence
  Description of Artificial Sequence Synthetic polynucleotide
cttgaatatattgatgatattcaacgattggggttgtcttattattttgaaaatgaaattgaacaaattttggagcaatttcataataattatcaaaatctaattgattttggtgataataacctttatgctgatgctctttgctttcggttgcttaggcaacaaggttataatattgcatgtgacatattcgacaagtacaagaatgaaaaaggaaaatttaaagaatcaatttcgagtgacattcgaggaatgttgaacttgtatgaagctgcacaaatgagagttcatggagagaaaatactagacgaagcacttatctttacaactactcatcttgaatcctcagttaaaacatgtcaattgagctctccttatctagacctagtgaaacatgccctaatgcaccctattcgaaagagcttacaaagaagagaggcaagactttacatatcactttatcatcaactactttctcatgaggagattcttttaacacttgctaaattagatttcaacttgcttcaaaaactacatcaaaaggaactaagttatattacgaggtggtggaaggagtttgattacaaaagtaagcattcatttataaaagacagaatagtggagtgctatttctgggtttatggagtgttttttgaggcagaaacttcccaaatcagactaataatcaccaaattaattgctattctcacaataattgatgatgcttatgatagctttggttcacttgaagaactagagccttttactcaagcaattgaaaggtgggatatatgtgccatagatactctgcctgagtacatgaaaatattttacatgaaacttttggagatctacaatgaaattgaacaattttctaaggaaagatcatactgccctagctatgctaagaaaggggtgcaatctctaattagagcttattttaaggaagccaaatggttacacacaaaatatataccaacattagatgaatatatgccagttggggttgatagtgcaggatcttttatgctcatttcaatggtttttattggaatgggagatattgttacaaaacattctatggattggatattttctaatcctcaacctaaaattatacaaactatggcaatagttggaagagttatgaatgacattggctaccataagaagaaatcatcaggagaaattgtggcttcaactgtggagtgttacatgaaacaatatggcgtgactggtgaagaagctatagagaaacttagcgaacaagttaaagattcatggaaagatctcaatgaagatcttctcaatccaatcactatctctaggccactcttaatgcaagttctaaagcttgtacgagtgaaccatgagatttatagagaaggagatggctttacacaacccactttgctcaagaatttgattcattctctcatcatcaatccaatt

SEQ ID NO: 1354 - DNA, 1593 bp, Artificial Sequence
  Description of Artificial Sequence Synthetic polynucleotide
atgaaggaaaataatgatgatagtgaaatagttattctgaagaaagaagtgaagaagaaaatacttgagcttgactatgttgaaaacccattagagacacttgaatttattgattcaattcaacgattgggagtgtcgtactattttgaaaatcaaattgaagtagttttgaagaacatatgtaacaaatttcacgaggaaaataatgacgacggtgatctttatgttgttgctcttcgatttcgattggttagacaacaaggttattacatgtcatgtgacgtgttcaacaagttcacgaacaatgaaggaaaattcaaagagtcactttttaacgatattcgaggaatgttaagcttatatgaggcttcacaactaagagtgcatggagagaaaatactagatgatgccctaatattcacaacaaatcacctcgaatctggagctaaaacctcgaaattaagctctcatatttcaaaccaagtaaatcacgcgttgaaaaaccctattcgaaagagcttgcaaaggagacaggctagacattacatgtctttataccatcaaatctcttctcacaatgagcatcttctagctctagctatactagatttcaacttgctacaaaaactttaccagaaggaattaagtgatctcacaaggtggtggaagaagtttgattatgaaagaaggcaatcgttctctagagaaagaataatggattgttacttttggacttttggagtgttttttgaggctcaaacctcacatattaggttgctgatgagcaagttgattgctttgctcactcttgttgatgatatttatgataactttggaacttttcaagagctacacctcttaactgaagcaatacaaagatatatatgtatatatatatatatgagtaggtgggatatgtgtttgattgatcaactaccagagtacattcaaccatgttacaaggaaatcttgaatctttacactgaaattggagaatttactaaggacaaatcatattgccttagctacgctaagaaaggattccaagagctagtaaaagggtactttgaggaagccaaatggttacaccaaaaacataatccaacactagatgagtatatgccaattgcacttgttactgctgcttctcctctactcattgctatttcatttattggaatgccaaatgttgttacaaaagattctatgaattggatcttttctcatccacagcctaaaagtgtgcggactctgtcgatcgttggtagagttcttaacgacattggcttccaccagtggagggaaaggagaacagagaaagtagatttttcagctgtggattgttatataagacaatatggtgtggaagaagaagaagcaatacaaagacttaaagaacaagttagtgattcatggaaagatctgaatgaagagtgtctctatcccaacaatatgaatatcccaatgccacttctaatgagagttcttgatcttgtacggatgaataatgaattatacagagaaggagatggttttacaagagaagctttcatcaaagatttgattgattctctcatcatcaatccctacatc

SEQ ID NO: 1355 - DNA, 1863 bp, Artificial Sequence
  Description of Artificial Sequence Synthetic polynucleotide
aatagtaataataataatgccttcactagatctgtgaaatcatgtttaagttcaaaccataataattataatattattcataataaaactgtattaatgtctaccaataataataataataataataatattatttctcgaagatcagcaaactatcaaccttcactttggcattttgattatgtacaatcactttctacccctttcaaggaaggagcatatgccaaaagagttgagaaagtaaaagaagaggtaagggtaatggtgaagagagcaaaagaggaagagaagcctttatctcaacttgagctaattgatgtattgcaaagacttggaatctcttatcactttgaggatgaaattaatgatatattgaaacatatatataacaacaataatgtgtacaacaccaacaataatgtgtatgccaattctcttgaatttagactcctaagacaacatggttatccggtgtctcaagaaatttttagtacgtgcaaagatgaaagaggcaattttatggtgtgtaccaatgatatcaaaggaatgttatctttatatgaagcttcattctatttggtagaaaatgaagatggtattttggaagagacaagagaaacaacaaagaaatatcttgaggaatacataatcatgatcatggaaaaacaacaatcattattagatcaaaataataataataatgataatgattatgattatgaactagtgagccatgcattggagcttccacttcattggagaatgttaagattggagagtaggtggtttattgatgtgtatgagaagagactagacatgaaccctactctacttaccttagctaaactagatttcaacattgtccaatcaatataccaagatgatcttaaacatgtcttcagctggtgggaaagcactggtatgggaaagaagttggaatttgcaagagatagaacaatggtgaatttcttatggacagtaggagttgcatttgagccacattacaaaaattttagaagaatgattacaaaagtaaatgctttaattacagtaatagatgacatatatgatgtttatggtacactagatgaattggagctcttcactaatgcagttgagagatgggatattagtgctatggatgggctccctgagtatatgaagacatgttttcttgctttatacaatttcataaatgatcttccatttgatgtgttaaaaggagaagaaggcctccatataataaaattccttcagaaatcgtgggcagatctttgcaaatcttatttaagagaagcaagatggtattatgatagatacacaccaagttttgaagagtacattgaaaatgcatggatatcaatatcaggacctgttatactatcacatttatacttttttgtagtgaatccaaacaaggaagatgccttattaagtacttgctttgatggataccctaccataatacgacattcatcgatgattttacgtcttacagatgatcttgcaacttcaacggatgaactgaaaagaggcgatgtttctaaatcagtccaatgcaaaatgcatgaagatggcgtgtttgaagaagaagctcgtcaacatattaagttattaataagtgaaacatggaagcttcttaataaagattacataaatttggatgatgatgatgatggtgatgactactctccaatgttctataagtctaataatattaataaggctttcattgaaatgtgtttaaaccttggtagaatggcacattgcatttatcaatatggagatggacatggaattcaagatcgccaaacaaaagatcatgtactatcattacttattcaccctattccacttacccaa

SEQ ID NO: 1356 - DNA, 1653 bp, Artificial Sequence
  Description of Artificial Sequence Synthetic polynucleotide
atgtcaaatattcaagtcttagcttcatctcaattatgtgacaaaattattgctcgcccaacaacaaaatttcacccttctatttggggtgatcgattcctccattacaatatttcagaacaagacttggtttgcaaacaagaaagagttgaagaattaatacaagttgtaaagaaagagatattatcttcaaatcatgatcaattgaagttgattgacaatctccaacgtttgggattatcatatcatttcgagagtgaaatcgagaaattattggaactattaagtatcggcactcatcatcaaaataataatctacatgatgtttctcttcggtttagactattaagacaaagtggatttaaggtttcatcaagcatatttgaaaaattcaaagacgagaaaggtaactttaaagaaagcctaataaccgatgtttcaggtttgcttagcttgtatgaggcttcgcacttgagttatgttggagaaagcatactagatgaagcacttgctttcacaaccactcaccttaaggctattgtggcaaataataaagatcatccattatcacatcaaatatccatagccttggaaaggcctctaagaaagaccatagagaggcttcatgctaggttttacatctcaatctatgaaaaggatgcctctcataacaaactattgctagagcttgcaaagttagacttcaatgtacttcaatgtttccacgaaaaggaacttagtgaaattatgaggtggtggaaggagcatgagtttgtaaagaaattcccttttgcaagagataggatggtggaactatatttttggatattgggtgtatattatgaacccaaatactctcgagcaagaaagcttttgaccaaagtcattgcattgacctcaatcactgatgatacttatgatgcatatggtactattgatgagcttgagcttcttaccaaagcaattcaaaggtgggacataacttgtatggataaacttgagccagaatatttaaagacatattataaggtaatgttggaatcttatgaagaatttgaaaaggagcttaaaaaggaagaattatacaaactcgagtatgcaaaagaagagatgaaaagaactattggaggttattttgaagaagctcgatggttgaatgaaggatatttcccaagcttcgatgagcatttgagagtttcatatgtttcttctgctaacgttttgttgatagccacaagttttgtaggaatgcatgatattgtaacacatgaaactctagattggctctctaaagaccctaagattgtttcagcttccactctcctctcaagattcatggatgacataggctctcgcaagtttgagcaagagagaaatcacataccatctacagtggattgttacatgaaacaatatggagtatcagaggaagaggcaattaaagaacttaataaaagagtgatcacccactggaaagaaattaatgaagactttattaggccaactgttgtgccttttcctatcttacttcgtgttcttaattttacaaaagtaatagatcttttttacaaagagggtgatgatcaatacacacgtgttggaaaagcgctcaaagaaagcattgatgctttgcttatagattcaatcccatta

SEQ ID NO: 1357 - DNA, 1668 bp, Artificial Sequence
  Description of Artificial Sequence Synthetic polynucleotide
atgtcatatcaagttttagcctcatcacaaaatgacaaagtatcaaaaattgttcgtccaacaacaacttatcaaccttctatttggggggagagatttctccaatatagtatttcagaccaagatgtaagctataaaaagcagcgagttgacgaattaaaggaagtggtaaggagagaggtatttttagaatgttatgataatgtttcatatgtgttgaagatagtggatgatgttcaacgtttgggattgtcgtatcattttgaaagtgaaatagaaagagcgctacaacacatttacgataacactattcatcaaaaccacaaagatgaagatttacatgacacttctacccgctttagactattgcgccaacatggatttatggtttcatctaatatatttaaaatatttaaagatgagaaagggaattttaaggagtgcttgataactgacattcccggtttgttgagcttatatgaggcctcacagttaagctatattggagagaatatattagatgaatcacttgctttcaccaccactcaccttcatcagtttgtgaaaaatgaaaaaacacatccattatcaaatgaagtattactagccttgcagagacctataagaaagagcctagataggctccacgctaggcattacatttcatcttacgaaaacaagatttgtcataacaaaacattgctagaacttgcaaagttggatttcaatctattacaatgtttgcatagaaaagagcttagtgaaatttccaggtggtggaaagaaatagactttgtacacaaactaccttttgcaagagataggattgtggagctatacctttggctattaggagtttttcatgaacctgaattttcacttgcaagaaatatttcaacaaaagtcattgcattggcctcagttgcagatgatatttatgatgcatatggtacatttgaagagcttgagctccttactgaatcaattaataggtgggacttaaattgtgcggatcaactacgtccagaatgtttacagacattttataaggttcttttaaattgttatgaagaatttgagagtgagcttggaaaggaggaaagttacaaagtttactatgcaagagaagcgatgaaaagattacttggagcatatttcagtgaagctcgatggttgcacgaaggatatttccctagtttcgatgagcatttgaaggtttcattgatttcttgtggatacaccatgatgatagtgacctctttaattggaatgaaagattgtgtaacaaaacaagattttgagtggctctcaaaagaccccaagataatgagagattgcaatatcctttgtaggttcatggatgacatagtctctcataagtttgagcaacaaagagatcattcaccgtctaccgttgagagttacatgaggcaatatggggtgtcagagcaagaggcatgtgatgagctaagaaagcaagtggttaattcatggaaagaaataaacaaagcattcctcaggccttctaatgtgccttatccagttttatctcttgttctcaatttttcgagagtaatggatcttctctacaaagatggtgatggctatacgcatgttggaaaagagacaaaaaatagtgttgttgcgttacttatagatcaaatccca

SEQ ID NO: 1358 - DNA, 1719 bp, Artificial Sequence
  Description of Artificial Sequence Synthetic polynucleotide
tcaagtgcatgctaccccatcatatgtgctgtggtcaatagttctaatgcaattattgatcgacgatctgccaactttgagccctccatttggtcttttgattatattcaatctcttacaagccaatataaggaagaatcttatacaggtcgagtcaaagaattggagatagatgtgaaaaagatgttaattgagatggaaaattctttaattcaacttgaactcattgacacattgcaaagacttggagtatcttatcgttttgaggatgaaataaatactattttgaaagaaaaatatattaatatgatcaataaccctaattataataatttatatgtcactgctcttgaatttaggcttctacgtcaacatggctatgcagtacctcaagagatttttaatcattttaaggaagagataggaaaaatcaaagcgaacatgattagtgatgatattatggaagtattggccttatatgaagcttcattctatgagaaaaaaggcgaaagtattttagaagaagttagaattttcacaactcaatatctaaagaactacattataatgatatcacaagaaagaaagttaatgattgataatgataatgattatgattatgatatcgaaatagtgaatcacgctttggagctcccacttcatcggaggactacaagaacagaagctaagtggttcatcgacgcgtatgagaaaaaacaagacatgaatcctattttgcttgagtttgctaaactcgatttcaacatgatacaatcaacacatcatgacgatctaaaacatatattcaggtggtggaggcatactaaacttggggagttgaattttgcaagagatcgattgatggaatgtttcttatggaaaattggaataagatttgagccaaaattcagttattttagaataacaactgccaaattatttgagctaataacagtaattgatgatatatatgatgtttatggaacattggatgaattagagcttttcaccaaagctattgagagatgggatatggaaatgataaatgagttaccagaatatatgaagatgccttatattgttttacacaataccattaatgaaatggtatttgaggtattaagagaccaacacatctccatcaacattcaataccttaagaaaacgtgggtagatatgtgtagaggttttttacaagaggcaaaatggtactatagtggatatacaccaacactagaagaatatattaaaaatggttggatttcagtgggagcaccggttctaattgtgcatgcttatttttctcatgcaaattataatcacacagttacaagtagtaaagagattttcgaatgctttgaacatggttattatcccgccataattcgtcatagtgccataatattacgacttacaaatgacctagcaacatcatcggaggaactgaaaagaggtgatgctccgacatcaattcaatgttatatgcaagaaaaaaaagtatctgaggagaaagctcgtgaacatattaagtttttaataagtgaaatatggaaagagatgaataatgatgttggattatatccaatctcattgattgaagatgctacaaattttgctaagatgggatttttcatgtatcagcatggtgatggtcatagttctcaagataatcaatccaaacaaaaaatttcatccttgattattgaacctattccctta

SEQ ID NO: 1359 - DNA, 1689 bp, Artificial Sequence
  Description of Artificial Sequence Synthetic polynucleotide
agtactccaatattcaaagatcaaccagcagcaattgtccggcgatcaggcaactacaaacccacattgtgggatgctcattttttcaattcactccaagtcatatatacggaggaaagctatgggaaaagaatcaatgagctgaaagaagatgtgagaagaattcttgagaaggaggctgaaaatcctttagttaagcttgaacagatcaatgatttgagcagacttggaatatcttatcactttgaaaatcaaattaaaacaatattaaacctgacatttaacaataataatgcattgtggaagaaagataatctgtatgccactgctcttcatttcaaactccttagacaatatggattcagtcctgtatcttcagaggttttcaatgtcttcaaagacgagaaaaatgagttcaaggagaatttaagtaaggatgtgaaaggaatggtatgtctatatgaggcttcattctattcatttaggggtgaacccatattagatgaagcaagagatttcacaaccaaacatctcaaacaatacttgatgacgaggcaatgtcaaactaaatctgttgaccatgatgatgatgatgatcttgtgaaacttgtggagcatgccttggatcttccactccattggagattgccaagattggaggctaggtggtttattgatatgtatgctgaaaggaattatgatatgaatccaacttttcttgactttgctaaacttgactataacttcgttcagtcagcataccaaaaagagctcaaatatatttcaaggtggtggagtggttctagattgacagaaaggctaccatttgctagagacagagtagtggaaattttctattctgcagtggcactgaaatatgaggcagaatttgggtttgttagaactgtgatgacaaaaattggtctcttgctaacactcatggacgatatatatgatgtgtatggtacactagatgagcttcagctttttctagaggcaattgagaggtggaatataaatgaactggatcagctacccgattacatgaagatattgtttgttgctttttataacaatgtgaatgagatatcttattatgtcctcaaagaaaatgggattcacaccatcaaatatctaaagaaagcgttgggtgatctgtgtaaatgctatatggaggaggcaaaatggtttcacagtggacacataccaagtttagaagaattcattgagaatggatggaaatcaattacaatacctctttgtcttatctatcattattgcttaattacaacttcaattacagaacaagacatggaacacttgcttcaatatccaaccattttacgtgtctcaggaactgtttttcgatttatagatgacttgggaacctcatcagatgaacttgaaagaggagataatccttcatcaattcaatgctacatgcgtgaaaagggtgttagtgaaaatgaaagccgtgaacacatatggaatttaataagtgaaggatggaaagaaattaatgaagttaaagcttcaacctctccatactctcaagtgtttattgaatctgccattgattttgttagaggagcaatggagatgtaccacaagggtgatggttttggtactaatcaagatcggtatcttaagaccaaagttgtcaacatgttcttcgatccaattccaatt

SEQ ID NO: 1360 - DNA, 1635 bp, Artificial Sequence
  Description of Artificial Sequence Synthetic polynucleotide
cgtcgatcggccaattttcatcctagtatctggggagatcactttctcaaatatacccatcaatcattggaagttgatgataatacaaagcaagaagttgaaaaactaaaagaggaagtgagagggatgctaactgctgcccccagcattaattcttcccaaaagctacagttaattgatacaatccaacggttaggagtcgcttatcattttgagagcgaaatcagtgacattttgggaaaaatgaagaaaatcaatcctcacactgaattggataatagtacccttcatacggtctccctttggtttcgattgcttagacaacaaggatacaacatatcaagtgatatatttgagaagtttacaaacaaaaaaggaaaactggacgatagtgttagtagtgatgttgaaggaatgctaagcttgtatgaagcttcacatatgagaatccatggagagcctatacttgaagaagcagtagttttcaccacaactcatttggtggaggcctctaaggagaaatcccagttgaccatgagctcattatttcttgcggcacaagtcaaccatgccttaagacagcccatcttgaagggcttgccaagagtagaaattcaacgctttatttctctctatcatcacgaccctaaccacagtcatcttttgcttcgctttgctaaattggacttcaacgttctccaaaagttgcaccagaaggaacttcatgaaatttcaaagtggtggaaagacttggactttgcatcgaagctacctttcgcacgtgacagactagtcgagggttatatttggcctgtgggagtatactttgagccaaagtacagtgctgctagggttatattgacaaaagtaattggtgtgacaacaatgattgatgacatttatgatgtttatggaactttagaagaactggaactcttcacagacgcaatagaaaagtgggatataagctgcagtgatcaactccctgattacatgaaatattgttacgaggcactgttaaaattatacgatgaaattgaggaggagttggccatgcaaggcagaacttatcggatggcctatgcaaaggagacaatgaagaagctagctcaatcctactacgtggaagctcaatggtttcataaaaagtacacaccaacgttacaggaatatatggaggttgcactggtgtcatccacgtactacatgctcactgccacgtcattcctaggaatgggagaagaagtctcagctgaggtctttcattggcttatgaactctcctaagattgtaacagcatcagcagtagtttgcagactcatggatgatgtggtcagccacaagtttgagcaagatagaggacacgtggactctagcgtggaatgttatatgaagcaatataatgtaagagaagaagaagcgtgcaaagaattgaagaaacaagttttagatgcatggaaagaaatgaatgaggagtgcatggagccaagagatgtgcccatgtctgtcctcatgcgcgtggtcaatctcggacgagtgatcgatgctgtttacaaagatggagacggttacacccatgccggaggaatcatgaagacttttgtcaaatctttgtttattcaaactcttcccctc

SEQ ID NO: 1361 - DNA, 1656 bp, Artificial Sequence
  Description of Artificial Sequence Synthetic polynucleotide
atgtcaaatattcaagtcttagcttcatctcaattatgtgacaaaattattgctcgtccaacaacaaattttcacccttctatttggggtgatcgattcctccattacaatatttcagaacaagacttggtttgcaaacaagaaaaagttgaagaattaatacaagttgtgaagaaagagatattatcttcaaatcatgatcaattgaagttgattgacaatctccaacgtttgggattatcacatcattttgagagtgaaattgagaaattgttggaacaattaagtatcggcactcatcatcaaaatcatcatgatctacatgatgcttctctttggtttagattattaagacaacatggatttaatgtttcatcaagtatatttgaaaaatttaaggacgctgaaggtaattttaagaaaagcttgataaccgatgtatcgggtttacttagtttgtatgaggcttcacacttgagttatgttggagaaagcatactagatgaagcacttgctttcacaaccactcaccttaaggctattgtggcaaatagtaaagatcatccattatcacatcaaatatccatagccttgcataggcctctaagaaagaccatagagaggcttcatgctaggttttacatctcaatctatgaaaaggatgtctctcataacaaagtattgctagaccttgcaaagttagatttcaatctacttcaatgtttccacaaaaaggaacttagtgaaattatgaggtggtggaaggagcatgagtttgtaaagaaattcccttttgcaagagataggatggtggaactgtatttttggatattgaatgtatattatgaacccaaatactctcgagcaagaaagcttttgaccaaagtcattgcattgacctcaatcactgatgatacttatgatgcatatggtactattgatgagcttgagcttcttaccaaagcaattcaaaggtgggacataaattgtatggataaacttgagccagaatatttaaagacatattataaggtaatgttggaatcttatgaagaatttgaaaaggagcttaaaaaggaagaattatacaaacttgagtatgcaaaagaagagatgaaaagaactattggaggttattttgaagaagctcgatggttgaatgaaggatatttcccaagcttcgatgagcatttgagagtttcatatgtttcttctgctaacgttttgttgatagccacaagttttgtaggaatgcatgatattgtaacacatgaaactctagattggctctctaaagaccctaagattgtttcagcttccactctcctatcaagattcatggatgacataggctctcgcaagtttgagcaacagagaaatcacataccatctacagttgattgttacatgaaacaatatggagtatcagaggaagaggcaattaaagaacttaataaaagagtggtcacctactggaaagaaattaatgaagactttattaggccaactgttgtgccttttcctatcttacttcgtgttcttaattttacaaaagtagcagatcttctttacaaagagggtgatgatcaatacacaaatgttggaaaagtgctcaaagaaagcattgctgctttgcttatagattcaatcccatta

SEQ ID NO: 1362 - DNA, 1668 bp, Artificial Sequence
  Description of Artificial Sequence Synthetic polynucleotide
atgtcatatcaagttttagcctcatcacaaaatgacaaagtatcaaaaattgttcgtccaacaacaacttatcaaccttctatttggggggagagatttctccaatatagtatttcagaccaagactttagctataaaaagcagcgagttgacgaattaaaggaagtggtaaggagagaggtatttttagaatgttatgataatgtttcatatgtgttgaagatagtggatgatgttcaacgtttgggattgtcgtatcattttgaaaatgaaatagaaaaagcgctacaacacatttacgataacactattcatcaaaaccacgaagatgaagatttacatgacacttctacccgctttagactattgcgccaacatggatttatggtttcatctaatatatttaaaatatttaaagatgagcaagggaattttaaggagtgcttgataactgacattcctggtttgctgagcttatatgaggcctcacatttaagctatattggagagaatatattagatgaagcacttgctttcaccaccactcaccttcatcagtttgtgaaaaatgaaaaaacacatccattatcaaatgaagtattactagccttgcagagacctataagaaagagcctagagaggctccacgccaggcattacatttcatcttacgaaaacaagatttgtcataacaaaacattgctagaacttgcaaagttggatttcaatctattacaatgtttgcatagaaaagagcttagtcaaatttccaggtggtggaaagaaatagactttgtacacaaactaccttttgcaagagataggattgtggagctatacctttggctattaggagtttttcatgaacctgaattgtcactggcaagaattatttcaacaaaagttattgccttggcctcagttgcagatgatatttatgatgcatatggtacatttgaagagcttgagctccttactgaatcaattaataggtgggacttaaattgtgcggatcaacttcgtccagaatgtttacagacattttataaggttcttttaaattgttatgaagaatttgagagtgagcttggaaaggaggaaagttacaaagtttactatgcaagagaagagatgaaaagattacttggagcatatttcagtgaagctcgatggttgcacgaaggatatctccctagcttcgatgagcatttgaaggtttctttgatttcttgtggatacaccatgatgatagtgacctctttaattggcatgaaagattgtgtaacaaagcaagattttgagtggctctcaaaagaccccaatataatgagagattgcaatatcctttgtaggttcatggatgacatagtctctcataagtttgagcaacaaagagatcattcaccgtctaccgttgagagttacatgaggcaatatggggtgtcagaacaagaggcatgtgatgagctaagaaagcaagtgattaattcatggaaagaaataaacaaagcattcctcaggccttctaatgtgccttatccagttttatctcttgttctcaatttttcgagagtaatggatcttctctacaaagatggtgatggctatacgcatattggaaaagagacaaaaaatagtgttgctgcgttacttatagatcaaatcccg

SEQ ID NO: 1363 - DNA, 1713 bp, Artificial Sequence
  Description of Artificial Sequence Synthetic polynucleotide
attacaggactaatctccactactactttcaaagaacaaccagcaatagtccgtcgatcaggaaactacaaacccccattgtgggatgctcattttatccaatcactccaagtcatatatacggaggaaagctatgggaaaagaatcaatgagctgaaagaagatgtgagaagaattcttgagaaggaggctgaaaatcctttagttaagcttgaacagattaatgatttgagcagacttggaatatcttatcactttgaagatcaaattaaggcaatattaaaccttacatataataataataatgcattgtggaagaaagataatctgtatgccactgctcttcatttcaaactccttagacaatatggattcaatcctgtatcttcagagattttcaatgctttcaaagacgagaaaaaggagttcaaggagagtttgagtaaagatgtgaaaggaatggtatgtctatatgaggcttcattctattcatttaggggtgaacccatattagatgaagcaagagatttcacaaccaaacatctcaaacaatacttgatgatgacgaggcaaggtcaaaatgttgaccatgatgatgataatgatttaatggtgaaattagtggagcatgctttggagcttccagtgcattggagaatgaaaaggttggaggcaaggtggtttattgatatgtatgctgaaatgtctcatcatcatcatatgaattcaacttttcttcaacttgctaaactagattttaacgttgtacaatcaacataccaagaagaccttaaacatgttgtaaggtggtggaaaactactagtctaggagagaggttaccctttgcaagagataggatagttgaaattttcttatggagtgtgggacttaaatttgagccacagtttagatattgccgaaaaatgctcacaaagattggtcaattggtaacgacaatggacgatatatttgacgtgtatggtacgcttgatgagctaagcctctttcaacacgcattgggaaggtgggatataaatacgatcgaccaacttccagattacatgaagatattttttttggctacttataatgtggtgaatgaaatggcgtatgacgtgctaaaacaaaatggcattctcattatcaaatatttaaagaaaacgtggacggatttatgtaaatgttacatgttggaggcaaattggtatcatagtggatacacaccaagcttggaagaatacattaaaaatggatggatatcaattgcagaacccttaattctagtcaatctttattgccttataaccaatccaataaaagaagatgacatagactgcttgcttcaatatcccacctttattcgtatctcaggaatcattgctcgacttgttgatgatttaggaacttcatcggatgaactgaaaagaggagacaatcctaaatcgattcaatgctatatgaaagaaaatggtatttgtgatgaaaagaatggccgtgaacacataaggaatttaatcagtgaaacatggaaggaaatgaatgaagctcgagtgggtgagtctccattttctcaagcttttattgaaactgccatagatttcgttaggacagcaatgatgatataccaaaaggagcaagatggggttggtactaatattgatcattacacaaaagatggaatcatttccttgttcttcacttccattcccatt

SEQ ID NO: 1364 - DNA, 1854 bp, Artificial Sequence
  Description of Artificial Sequence Synthetic polynucleotide
atggcttttcaccaatttgctccatcatcatccctccctatttggagtagaattaataatcgttttacaccaaaaacttctattattacttctatttcaaaaccaaaatcaaacttgaaatcgagatcgagatcaagtacttgctaccccatacaatgtactgtggtcgataaccctagttctacgattactaataatagtgatcgaagatcagccaactatggacctcccatttggtcttttgattttattcaatctcttccaatccaatataagggagaatcctatacaaggcaattaaataagttgaagaaagaagtgacaagatggcttggactggaaattaactctttagccctacttgagctaattgattacattgcaaaggcttggaatagcttaccatttaagaatgaaataaacactattttgaaggaaaaatacacggataattatattaacaataaaattattactaaccctaattataagaatttatatgccattgctcgcgaatttaggcttctacgccaacatggctatacagtacctcaagattttaatgctttcaaggataagagagggaaatttaaaacatgcttgagtgatgatattatgggagtattgtgtttatatgaagcttcattctatgctatgaaacatgaaaatattttggaggaagcaagaattttttcaactaaatgtcttaaaaaatacatggagaaaatggagaatgaggaagagaaaaaaatattattagatgataataatattaatagtaattgctataattaatcacgctttcgagcttcgcttcattggagaataacaagatcggaagctaggtggttcattgacgaaattatgagaaaaaacaagacgtgaattcgacttgtttcgaggttgcgaattagattttaatatagggcaatcaaacaccaagaagatttacaacatttatcgaggtggtggaggggattgtaaacttggtgggaaatgaattttgctagcgatagattgatggaagctttcttatgggatgttggaaaattcgaggagaatttcagctattttagagaataaatgcaagattatttgtgctttataacaataattgatgatatttatgatgtatatggaactttggaggaattagagcttttcactagtgctgttgaaagatgggatgtgaaattaatcaatgagttaccagattacatgaagatgcctttctttgttttaaataccataaatgagatggggttcgatgtattagtacaaaactttgtcaacattgaataccttaagaaatcggtatgtagattatgtaaatgttatttacaagaagcaaaatggtattatagtggataccaaccaacattggaagaatacactgagttgggttggctttcaataggagcatcagtaattcttatgcatgcttattgtttcacaaatcctataacaaaacaagacttgaaaagtttgcaattgcaacattatcccaacataattaaacaagcatgcttgattacaaggcttgcagatgatttaggaacatctcgggatgaattgaatagaggcgacgttcctaaatcgattcaatgttacatgtacgataataatgctaccgaagacgaagctcgtgaaatcaagttcttgataagtgaaacatggaaggatatgaataagaaagatgaagatgagagttgtttatcagaaaattttgttgaagtttgcaaaaatatggctagaacagcactattcatatatgagaatggagatggacatggttctcagaatagtttatcaaaagaacgtatttcaaccttgattattactccaattaatatc

SEQ ID NO: 1365 - DNA, 1695 bp, Artificial Sequence
  Description of Artificial Sequence Synthetic polynucleotide
agtactccaatattcaaagatcaaccagcagcaatcgtccgccgatcaggaaactacaaacccacattgtgggatgctcattttttccaatcactccaagtcatatatacggaggaaagctatgggaaaagaatcaatgagctgaaagaagatgtgagaagaattcttgagaaggaggctgaaaatcctttagttaagcttgaacagatcaatgatttgagcagacttggaatatcttatcactttgaagatcaaattaagacaatattaaacctgacatttaacaataataatgtattgtggaagaaagataatttgtatgccactgctcttcatttcaaactccttcgacaatatggattcagtcctgtatcttcagaggttttcaatgccttcaaaaacgagaaaaaggagttcaaagagagtttgagtaaggatgtgaaaggaatggtatgtctatatgaggcttcattctattcatttaggggtgaacccatattagatgaagcaagagacttcacaaccaaacatctcaaacaatacttgatgacaaggcaaagtcaatttactagagttgaccatcatgatgatgatgatcatgatcttgtgaaattggtagagcatgccttggatcttccactccattggagattgccaagattggaggctaggtggtttattgatatgtatgctgaaaggaattatgatatgaatccaacttttcttgactttgctaaacttgactataacttcgttcagtcagcataccaaaaagagctcaaatatatttcaaggtggtggagtggttctagattgacagaaaggctaccatttgctagagacagagtagtggaaattttctattctgcagtggcattgaaatatgaggcagaatttggatttgttagaactgtgatgacaaaaattggtctcttgctaacactcatggacgatatatatgatgtgtatggtacactagatgagcttcagctttttctagaggcaattgagaggtggaatataaatgaactggatcagctacccgattacatgaagatattgtttgttgctttttataacaatgtgaatgagatatcttattatgtccttaaagaaaatgggattcacatcatcaaatatctgaagaaagcgttgggggatctgtgtaaatgctatatggaggaggcaaaatggtttcacagtggacacataccaagtttagaagaattcattgagaatggatggaaatcaattacaatacctctttgtcttatctatcattattgcttaattacaacttcaattacagaacaagacatggaacacttgcttcaatatccaaccattttacgtgtctcaggaactgtttttcgatttatagatgatttgggaacctcatcagatgaacttgaaagaggagataatccttcatcaattcaatgctacatgcgtgaaaagggtgttagtgaaaatgaaagccgtgaacacatatggaatttaattagtgaaggatggaaggaaattaatgaagttaaagcttcaaactctccatactctcaagtgtttattgaatctgccattgattttgttagaggagcaatggagatgtaccacaagggtgatggttttggtactaatcaagaccgatatcttaagaccaaagttgtcaacatgttcttcgatccaattccaatt

SEQ ID NO: 1366 - DNA, 1761 bp, Artificial Sequence
  Description of Artificial Sequence Synthetic polynucleotide
atgcaaatggcaggagtgaagttcattagctactcatctttatactcggctactttctcaaagaggccatcgaagaagaattataagcttcctcgttttggtaatccattattcatgtcaacaaaatgccagagtatatcatcatcatcatcaccaccaccattattgtcaatagatcatgatccaggggacatagaatggattttattgaataattagatggtcgctgattattatgacaattataagagtactacctcggaagagctgttgatcaaaaaggttgaaatggcattacgaaaacctactgatgatcaaagttacaaaatgaggctcatagatagcatccaacggttgggagttggccattatttcgaagaagaaatcaaagtaatccttcagatgttgtcagatttcaattccggccaagatgacctctttaacacggggcttcgttttcggctactcaggcataatggctttccaactacctcagatgtttttgacaaattcatcaaccaaaaaggagaaatagaggatgtaataatgggccaagacacattgggtatgctgagcttgtatgaggcatcctaccttgcagcaaattgtgaagaatcattggtgaaggcaatggaattcacaagatctcatctgaaaagttcgatgcctttcataacccaaaagctacaaaatgaagttgccaaagccttggagctccctagacacctgaggatggcaccattagaagctcgaaactacatcgacgaatatggcaaagaattaaaccactgccctgctcttcttgacttagccaagttagaatttaacgagcttcagtcactccacaaaagagaattaaccgagatcataaggtggtggaaacagttgggtctggttgagaaacttggttttgcaagagatcgacccttagaatgttttctatgggtagtgggaatattccctgggaaatgctattccaacgtccggattgagctggccaaaactgtttcaatcttactagtcattgatgatatctacgacacttacggatctttggatgaactccacttgttcaatcatgcaattctaagatgggatcttggtgcaatggacaagcttcctgagtacatgaaaatatgttacatggcactatacaatactactaatgaaattggctacagagttctcaaagaacatggattatgcgttacacaacacttaaagaaagtgtggttagacatatttgatgcatttctaaccgaagcagaatggttcgacaaaaaatacactccaactctagagcaatatttaacaaacggcgtaactagtggaggatcctacatggccttagtgcactcatttttcctcataggtcatggtattacagaccaaactatatcaatgatgcacccatatcctgagatcttctcccactcaggcaaaattctaagactttgggatgatttgggaacagccaaggaggagcaagagagaggagatgttgcttctagcatagattgttatatgaaagaagagaatattgaatcggaagatgaggccagaaaacacatcaagaaactgattagaagctcgtggatagagctaaacggagagttgaaggctcctagtgcattgcctcggtcgatcaccacggcctgttttgaccttgctagaacagcccaagtcatctatcaacatggagatgatcaaagctttttaagcgtagaagatcatgtacaatctttgttttttcgaccctgt

SEQ ID NO: 1367 - DNA, 1710 bp, Artificial Sequence
  Description of Artificial Sequence Synthetic polynucleotide
atgtccaaccaaataatattggcatcatcaaaaaatgagaaaatacataaaattgttcgaccaacaaaaaattatcatccatctatttggggagatcgatttctacattacaatatttcagaagaagagttgagttataaacaaggccaagttgaagaattgaaagaagtagtaagaaaggagatatttggagaattattatgtgatgattggaataatcgattgaagttaattgatgtggtagaacgtttgggattgtcctatcattttgagagtgaaatacaaaatgagcttcaacaaatttacaacaaaataaggattaatgatactaatttaaaacatgaaactttgcatgatgcttctattcggtttagacttctacgacaacatggatacagggtttctttagatatatttgacaagttcaaagatgagaatggcaactttaaagaatgtttggcgagtgacactcttggtttgcttagcttatatgaggcctcacatttgagttgcgtcggggaagatttactaaatgaagccttgtcttttacgaccagacacctaattgagtttttggcaaataataaaaaagaacaatgcgatgatgatgatgatcttttatcgaaagaaatatctcgagccctggagagacccttaagaaaaaccctaaacaggctccaagctaggcatttcatttcaatatatgaaaaagtggccttacataaccaagcattgctacaacttgcaaaattagatttcaatttattacagtccatgcacaaaaaggagcttagcgaaaactccaggtggtggaaagaatcagactttgtacacaaattcccttttgcaagagacaggagtgtggaactatacctttggatattagcagtctattatgaaccccaatactatttggcaagaataattttattcaaaattatttcctttgccacaatagctgatgacatttatgattcatatggtacttttgaagaacttaaactcctaactgatgtagttaaaaggtgggacataaatgagattgataaactcaatccaggatgttttcaagtattttataaagaacttttgatttgttatgaagaatttgagcaagggcttacaaaggaagaaacttacagagttcattacgcaaaagaagagtttaaagaatttctccaaggttatctggatgaagcttggtggttaaagaaagctgaacgcattccaagttttgatgagtatttgaaatttggtttggtcaattgtggttacttaatgttgacagtgagctctttaattggaatgaaaagtaacaatattgtaaccaaacaagtttttgagtggctctccaaaggccccaagattatcaatgcatccactactatttgtcggcttatggatgacatagctgaacacaagaatgagcaagagagaaatcatgtaccatctacaatagaatgttacatgaaacaatatggcgtatcagaagaagaaacatgtgatgaaatgaataggcgagtggttattgcatggaaagaaataaacgaagagtttctgaaaccaactgaagcagcttcacctatattagttcgtgccctgaatcttgcaagggttatagatcttctctacaaaaatggtgataactatacacaagttggaaaagttaccaaagataacgttgctgtcttgctcattgattcaattcca

SEQ ID NO: 1368 - DNA, 1857 bp, Artificial Sequence
  Description of Artificial Sequence Synthetic polynucleotide
atagcttttcaccaatttgcttcatcatcatccctccctatttggagtagtattgataatcgttttacaccaaaaacttctattacttctatttcaaaaccaaaaccaaaactaaaatcaaaatcaaacttgaaatcgagatcgagatcaagtacttgctaccccatacaatgtactgtggtcgataaccctagttctacgattactaataatagtgatcgaagatcagccaactatggacctcccatttggtcttttgattttgttcaatctcttccaatccaatataagggtgaatcttatacaagtcgattaaataagttggagaaagatgtgaaaaggatgctaattggagtggaaaactctttagcccaacttgaactaattgatacaatacaaagacttggaatatcttatcgttttgaaaatgaaatcatttctattttgaaagaaaaattcacaattaataatgacaaccctaatcctaattatgatttatatgctactgctctccaatttaggcttctacgccaatatggatttgaagtacctcaagaaattttcaataattttaaaaatcacaagacaggagagttcaaggcaaatataagtaatgatattatgggagcattgggcttatatgaagcttcattccatgggaaaaagggtgaaagtattttggaagaagcaagaattttcacaacaaaatgtctcaaaaaatacaaattaatgtcaagtagtaataataataatatgacattaatatcattattagtgaatcatgctttggagatgccacttcaatggagaatcacaagatcagaagctaaatggtttattgaagaaatatatgaaagaaaacaagacatgaatccaactttacttgagtttgccaaattggatttcaatatgctgcaatcaacatatcaagaggagctcaaagtactctctaggtggtggaaggattctaaacttggagagaaattgcctttcgttagagatagattggtggagtgtttcttatggcaagttggagtaagatttgagccacaattcagttactttagaataatggatacaaaactctatgttctattaacaataattgatgatatgcatgacatttatggaacattggaggaactacaacttttcactaatgctcttcaaagatgggatttgaaagaattagataagttaccagattatatgaagacagctttctactttacatacaatttcacaaatgaattggcatttgatgtattacaagaacatggttttgttcacattgaatacttcaagaaactgatggtagagttgtgtaaacatcatttgcaagaggcaaaatggttttatagtggatacaaaccaacattgcaagaatatgttgagaatggatggttgtctgtgggaggacaagttattcttatgcatgcatatttcgcttttacaaatcctgttaccaaagaggcattggaatgtctaaaagacggtcatcctaacatagttcgccatgcatcgataatattacgacttgcagatgatctaggaacattgtcggatgaaatgaaaagaggcgatgttcctaaatcaattcaatgttatatgcacgatactggtgcttctgaagatgaagctcgtgagcacataaaatatttaataagtgaatcatggaaggagatgaataatgaagatggaaatattaactcttttttctcaaatgaatttgttcaagtttgccaaaatcttggtagagcgtcacaattcatataccagtatggcgatggacatgcttctcagaataatctatcgaaagagcgcgttttagggttgattattactcctatccccatg

SEQ ID NO: 1369 - DNA, 1782 bp, Artificial Sequence
  Description of Artificial Sequence Synthetic polynucleotide
gtattttttagaccaaaattatatactaattataatattattaatggtggtacaaaatcaagattatcaagtgcatgctaccccatccaatgtgctgtggtcaatagttctaatgcaattattgatcgacgatcagccaactttgagccctccatttggtctttcgattatattcaatctcttacaagtcaatataagggagaaccttatacaagtcgagtgaaaaaacttgagagagatgtgaaaaaaatattagttgagatggaaaactctttagctcaacttgaactcattgatacattgcaaagacttggaatatcttaccgttttgagaatgaaataaattctattttgaacaaaaaatatctcaatattaataatcctaattataatttatatgctattgctcttcaatttaggcttctacgtcaacatggctatgcagtacctcaagaaatttttaatcagttgaaggacgagatagaaaaaatcaaagaaaacataaatggtaatgatatcatgggaatattagccttatacgaagcttctttttatgagaaaaaagacgaaagtattttaaaggaagctagaattttcacaactaaatgtctcaaaaactacactataatgatatcagagcaaaaaaagttaatgattgataatgattatgattatgatatcgaagtagtgaatcatgccttagagctcccacttcatcggaggaccacaagaacagaagcaaagtggttcattgacgcatatgcgaaaaaacaagacatgaatcctatgttgcttgagttagccaaactcgatttcaacatagtacaatcaacacatcatgaagatctaaaacatatattcaggtggtggagacatactaaacttggagagaaattgaattttgcaagagatcgattgatggaatgtttcttatggaatattggaataagatttgagtcaaaattcagctattttagaacaaaaactgccaaattatttgagctagtaacatttatagatgatatatatgatgtttatggaacattggatgaattagagcttttcaccaaagctgttgagagatgggatgtgaaaatgataaatgagttaccagaatacatgaagatgccttatcttgttttacacaataccataaatgatatggtatttgaggtgttaagagaccaagaaatctccatcaacattcaataccttaagaaaacgtgggtcgatatgtgtaaaagtttcttgcaagaggcaaaatggtactatagtggatacacaccaacattggaagaatatattgaaaatggttggatttcagtaggagcaccagttattcttgtgcatgcttatttttttcacgcaaataataatggcacaattacaaatactaaagagattttcgaatgcttggaatatggttattatcctgccattattcgtcacagtgccataatattacgatttacaaatgacctagcaacatcatcggaggaattgaaaagaggtgatgctccgacatcaattcaatgttacatgcaagaaaaaaatgtatctgaagaggaagctcgtgaacatattaagtttttaataaatgaagcatggaaggagatgaataatgatgttggattatatccaatctcattgactgaagatgctacaaactttgctaagatgggatttttcatatatcaacatggtgatggtcatagttctcaagataatcaatccaaacaaaaaatttcatccttgattattgaacctattcccctatataca

SEQ ID NO: 1370 - DNA, 1701 bp, Artificial Sequence
  Description of Artificial Sequence Synthetic polynucleotide
atgtcgtctcaaatcttagcaacctcatccaaaaatgacaatatacataaaattgttcggccaacaacaaattatcatccttctatttggggagaccgatttttgcattacgatattccaaaagaagaattgaattataaacaaggccaagttgaagaattgaaagaagtggtaagaaaggagatatttggagaattattatgtgatgattggaataatcgattgaagttaattgatgtggtagaacgtttgggattgtcctatcattttgagagtgaaatacaaaatgagcttcaacacatttacaacaaaataagcattaatgatactaatttcaaacatgaaactttgcatgatgcttctattcggtttagacttctacgacaacatggatatagggtttctttagatatatttgacaagttcaaagatgagaatggaaactttaaagaatgtttggcgagtgacaccgttggtttgcttagcttatatgaggcctcacatttgagctgtgttggagaaaatttactagatgaagccctttctttcactaccaaacacctaactgaatttttggaaaataataaaaaagaacaccccaatgatgatcctttatcaaaggaaatatctcgagccctagagaggcccttaagaaagaccctagtgaatctccatgcaaggtatttcatttcaatatatgaaaaagatgcctcacataacaaagtattgctacaacttgcaaagttagatttcaatctattacaatccatgcacaaaaaggagcttagtgaaatctccaggtggtggaaggaattagacagtgcacacaactttccatttgcaagaaataggattgtggaactatacatttggatattaggagtctattatgaaccccaatactcttttgcaagaaatattttagtgaagatcatagcactttcctcaatcgctgatgatatttatgattcatatggtatatttgaagaacataagctccttattgaagcaattgataggtgggacaaaaattgtatggataaactccatccagaatacttgcagaaatattataagatacttttgcaatcttttgaggaatttgaacaagagtttgaaaaggaggaaacttacaaagtttactatggaaaagaaacgtttaaaagattattaagaggttattttgaggaagctcgatggttgaatgaaggatacatgccaagtttggaggagcatttaaaagtttctttggttacttctggttatttcatgttgatggcttgctctttagttggaatgaaaagtaacaatattgtaaccaaacaagttttcgagtggctctccaaagaccccaagattgttagggcaagcgctagtgtttgcaggtacatggatgacgtggctggtcacaagaatgagcaagagagaaatcatataccatctacaatagaatgttacatgaagcaatatggtgtatcagaagaagaagcatgtgatgaaatgaataggcgagtggttattgcatggaaagaaataaacgaagagtttctcaaaccaactgaagcagcttcacctatattagttcgtgctctgaatcttgctagggttatggatcttctctacaaaaatggtgataactatacacaagttggaaaagtcactaaagatagtgttgctgtcttgctcattgatccaattcca

SEQ ID NO: 1371 - DNA, 1935 bp, Artificial Sequence
  Description of Artificial Sequence Synthetic polynucleotide
atggctgcattagtgtccactgtaagtagtattattaggtggaatagtaataataataataataataataccttcactagatctgtgaaatcatgtttaagttcaaattaccataataataataatattattcatagtaataaaactgtattgatgtccaccaataataataataataataataataagaatagttctcgaagatcagcaaactatcaacccccactttggcaatttgattatgtacaatcactttctactcctttcaaggatggagcatatgtcaaaagagttgagaaagtaaaggaagaagtaagagtgatggtgaagagagcaagagaggaggagaagcctttatctcaacttgagctaattgatgtattgcaaagacttggaatctcttatcactttgaggatgaaattaatgatatattgaaagttatatataacaacaataatgtgtacaacaccaacaaaaataatgtgtatgccaattctcttgaatttagactcctacgacaacatggttatccggtgtctcaagaaatttttagtacgtgcaaagatgaaagaggcaattttatggtgtgtaccaatgatatcaaaggaatgttatctttatatgaagcttcattctatttggtagaaaatgaagatggtattttggaagagacaagacacaaaacaaagaaatatcttgaggaatacataatcatgatcatggaaaaacaacaatcattattagatcaaaataataataatgattatgattatgataattatgattatgaactagtgagccatgcattggagcttccacttcattggagaatgttaagattggagagtaggtggtttattgatgtgtatgagaagagactagacatgaaccctactctacttaccttagctaaactagatttcaacattgtccaatcaatataccaagatgatcttaaacatgtcttcagctggtgggaaagcactgatatgggaaagaagttggaatttgcaagagatagaacaatggtgaatttcttatggacagtaggagttgcatttgagccacatttcaaaagttttagaagaatgattacaaaagtaaatgctttaattacagtaatagatgacatatatgatgtttatggtacactagatgaattggagctcttcactaatgcagttgagagatgggacattagtgctatggatgggctccctgagtatatgaagacatgttttcttgctttatacaatttcataaatgatcttccatttgatgtgttaaaaggagaagaaggcctccatataataaaattccttcagaaatcgtgggcagatctttgcaaatcttatttaagagaagcaagatggtattataatgaatacacaccaagatttgaagagtacattgagaatgcatggatatcaatatcaggacctgttatactatcacatttatacttttttgtagtgaatccaaacatggaagatgccttattaagtacttgctttgatggataccctaccataatacgacattcatcgatgattttacgacttacagatgatctgggaacttcaacggatgaattgaaaagaggcgatgttcctaaatcaatccaatgcaaaatgtatgaagatggtatatctgaagaggaagctcgtcaacgtattaagttattaataagtgaaacatggaagcttattaataaagattacataaatttggatgatgatgatgatggtggtgatgactactctccaatgttctataagtctaataatattaataaggctttcattgaaatgtgtttaaaccttggtagaatggcacattgcatttatcaatatggagatggacatggaattcaagatcgccaaacaaaagatcatgtactatcattacttattcaccctattcctcttacccaa

SEQ ID NO: 1372 - DNA, 2541 bp, Artificial Sequence
  Description of Artificial Sequence Synthetic polynucleotide
gaattccaatctttgccccaaactcaggctaagttgatcaacgagatcaaagaaacgatgttctcatcgttagacatcaatccttactccctcgtttctccttgtgcttacgacactgcatggctagccatgataccacatcctaaccaaccttcgaagcctatgttcgagggttgtttgagttgggtgcttaacaaccaaaccgaacatggcttctggggtaactgtgatgaccagagtggcatgccaaccctagagtgtctgactgccactctggcttgtgttgttgccctaagaaagtggaatgttggttcttccatgataagtaaagggttggaatttattcattcatctaatgcaaaaaggctcttaaaagaaatgaaaaaagaaggctttattcctcaatggtttgcaatcgttttcccgggaatggttgaattggctgaagaagttctcaaaatccaaatcttaaaggatcaatccgttttcgtttcggacatattttaccataggcaactcattttcaaaaagtacatatatttagagctacataataaggagacatatttgttatcatatcttgaagtgttgccatcatcatattttaatgaagaattaataataaaaaaattgtgtgaaaagggttctttgttccaatctccatcggcaacagcacaagctttcatggcaactgaaaattccaaatgcttgcattatttgcaaaccctagttcacaagttttccaataataacaacaataatatcattggagttcccactacatatcccatggacaaagatcttataaagctttgcattatcaactatgtcgaaagattagggttagctgagcatttcgctatcgaaatcgaacaacttctacaacaagtttataagaattatgtgaaatgcgatggagaattttattacgaaaaaagttactattcattggcaactttagagttacacctactcaaagaatcattagcttttaggcttctaaggatgcatggctacaaagtttttcctagtaacatctattggattttgaagaatgaggatataaaaaatcacatagaatcaaactatgaatgcttttcggttacgatgttgaatctttacagagctactgatcttgcttttcatggtgaatttgagcttgatgaactaagaatattttcgagaaaactacttcaaaagtctattttagtaggagctccacatacaaatccttttaacaaactgattgagaatgagttaagtcttccatggatggctcgactagatcacttggaacatagactttttattgaacaaactaatgaagctcaatcatctttatggatgggaaagacttctttccaaaggttgtcgaggtttcacaacgacaaactagttcgtctagccaccttaaattatgagttcaaacaatctatttacaagaccgaacttgaacaattaacaaggtggtgtaaatattggggacttaatgaaatgggatttggtagagaaaaaagtacatactgttattttgcagtagcttcagcttgttgttctttgccttatgattctccaattcgattgatggttgcaaagggtgctataataataacaattacagatgatttttttgatatgaaagaatctctaatcactgagttaaaaacttttactaaggcctttcaaaggtgggatggtaaagaattgagtggtgttagtaagaaaatatttgatgccctagataatctttggtatgaaacgatttgttcatggcttactgaatcggagtggagtaaaaatgggattgtaccaaccatggatgaatatcttaaagttggaatgacttccatagcaactcacaccttgcttcttccagcttcttgttttgtaataaatccaactttaccggtctactctcaattacgaccaattcaatgtgagagtgttacaaaactcgtcatgactatttgtcgtctgttaaatgacttacaaagttatgagaaggaaaaagaagaaggcaaaccaaactcaattactgtgtacatgaagaacaattctgaggtagaaatggaagaagcagtaaagtacgtgaaagagatattaaacaagaagaagaaagagttactcgaacatgttatgattgatggctttaccaatctttcaaaagagtgtaggcttctacacctctcttgcttaaaagtatttcaaatgttcttcaactcttcgaatcgatatgattcagacactgaaatgcttgacgacattagcaaagcaatctatgttcctttgaaatcaattgatcatgaaggactcgaaaagaatctcttaaggcctccaaagtcaccgatgattgatcttactcgagtcaaatcaacgaccatgaaatgttcgatgaacatgaataaatttagtcaaccctctaaatgtctaaccgctaaatattttgtcatgaatgagctttctcttcataaacacaacaaggttgagaacttgaaaataactacacctactatgccttcaaaactcaattttttctttatt

SEQ ID NO: 1373 - DNA, 1683 bp, Artificial Sequence
  Description of Artificial Sequence Synthetic polynucleotide
atgtctagtcaagtgttagcttcatctcaaaaaaatgacaaaacacaaaacatcattcgaccaacaacaaaatttcacccaccaatttggggagatcgattcctccattacaatatttcagaacaagagttggaatataaagagggacaagttgaagaattgaaagaagttgttaggaaagagatattccatggaaataataaaagaaatattattaatgtttcgaaacaattaaagttaattgatgatgtggaacgtttgggattatcttatcattttgaaagtgaaatagaaaaaaagctacaacacatttatgatcaacactactactactccaaccatgatgaagatctacatgatgtctctattcgatttagattactacgacaacatggatttaacatttcatctacagatatatttgagaaattcaaagatgagagtggtaagtttaaggaaagcttgaaaagtgacattgaaggtatgcttagcttgtatgaagcttcatacttgagctatgttgaagaaaatatactagatgaggctcttgctttcactaccaccaatcttaagttagtggccaacaaaaaagaacatccattatcacatgaaatatccttagccttatataggcctttgagaaaaaccctagtgaggctttatgctaggcattacatttcaatctatgagaaacaaccttctcataacaaagtattgctacaatttgcaaagttggacttcaatctactacaatctttgcataagaaggagcttagtgaaatttccaggtggtggaaagaattagacttggcaaacaaactaccatttgcaagaaataggattgtggagctatacctttggatactaggagtctttcatgaacctcaattctctcttgcaagaaaaatcttaattaaagccatttcaatggcctcagttgcagatgatatttatgatgcatatggtacatttgaagaacttgagctcctcaccgaagcaattctcaggtgggacataagttttatagataaacttagtccagattatttgaagacatattataaggtatttttgaattcttatgaagaatgtgaaaaagatcttaaaaaggaagagagatacaaacttcactatgcaaaagaatcgatgaaaaaattaatacaagcttatttccatgaagcacaatggttgaaccaaggacatttcccaagctttgatgagcatttgaaagtttcttttgtgtcttctggttacccaatgttgattgagacctcttttgttggaatgcaagatgttaaaacaaatcaagtatttgaatggctctctacacaaccaaagatttttagagcttgcactatcatttctagattcatggatgacctagtttctcgtaagttcgagcaagagaggaatcacgtaccatctacagtagattgctacatgaagcaatatggtgtatcggaacaagaggcatgtgatgagctcaataaacaagtggttaatctatggaaagaaataaaccaagagtttcttaggccaacttctatgccttcatctatcttagttagaattcttaatttcacaaaagtgcttgacattatctacaaagaaggtgatggctatacacatgttggaaaattggttaaggacagtgttgctgcgttgcttatagatcccatccca

SEQ ID NO: 1374 - DNA, 1329 bp, Artificial Sequence
  Description of Artificial Sequence Synthetic polynucleotide
tggttcttacataacgaagaaattaaaaatcacatcgagacaaactgcgaatacttttcgattatgttgttaaatctttacaaagccacagatcttgcttttcaagatgaatttgagcttaatgaagcaagaaatttttcaagaaaactacttgagaaatgtatttcaatggaagaaaatggagatcacatatttcataatctgattgagcatgaattaagtcttccatggatggcccgattggaccacctcgaacataggttttggatagaagaaactaaaaaccatgttctatggcttggaaagaatcattttcgaaggttgttaagcattcataatgacaaattaattcgtctagccatcttaaattaccacttcaaacaatctatcttcaaggctgaactcgaacacttaacaaggtggtgtaaagattggggacttagtgaaatgggatttggtagagaaaaaagtatgtattgttatttcgcaatagcttcaacttgtagttctttgccttatgattctccaattcgattggcggttgcaaaaggtgctataataataacggtgacagatgatttttttgatatgaaagactccttaatcaccgagttagaaaactttactaaggcaattcaaaggtgggatggtgaaggattgagtggtgttagtaagaaaatatttgatgccctagataattttgtaaaggaaatggcaattatgtaccttgatcaacaagaaacaagtgacatcacaaatttcttaagagaaatatggtatgaaacaattgtttcatggcttactgaatctaaatggagtaaaagtgggattataccaaccatggatgaatattttaaagttggaatgacttctatagcaactcacaccttgcttcttccagcttcatattttctaaagccaagcttaaaaatatcccaattacaatcaacggagtatgccatcgttactaaattggttatgattatttgtcgtttgttgaatgacatacaaagttatgagaaagaaaaaaatgagggcaaaccaaactcaattacagtgtacttgaagaacaatcccgaggtagagatcgaagaagcaatcgagtacgtgcaagagatactaaataaaaagaagaaagaattacttgagcatgttttgattgacaacgacgacgatgatgatgttagggttttgcacctctcttgcttaaaagtatttcaaatgttcttcaactcttcgaatcgatttgattcagaggttgaattgcttgatgatattggcaaagcgatctatattcctttgaagttagatggattagcatctaataaa

SEQ ID NO: 1375 - DNA, 1515 bp, Artificial Sequence
  Description of Artificial Sequence Synthetic polynucleotide
gatttagataaatacagggacgttttaagaaaagcagacccatttgatgaaggtctgaagatgattgatgctattcaacggctaggaattgactatatcttcgaggaagagattgataaaattatacaaagccagtctgcttataagttttttagagaattcgagcatgatcatcatcatcatcaagatctgtatgatgtagctcttcgttttcgactcttgagacaacacggtcacttcgttcctgctgatattttcaacaagtacaaggacaataataagggttgtttcgacacaaggctaagagaggacatcaagggtttgctgagcttatatgaagcctcccatctatgcatagagggagaaaatatccttgatgaagctgctttatttagtgctcaacacttggaagcgtccatgacacgtcttcatcgttatgatcaatatcaggccaaatatgtggcaactacacttcaaaatcccactcacaaaagtttatccaagttcacagccaaggacctttttggtgtctatcccagtcagaatgggtacataaatttgtttcaacaactagcaaaagttgaatttaatagagttcagtccctgcatagaatggaaattgataaagtcaccagatggtggcgagacattggtttagctaaggaattaactttcgcaagagatcaaccggtgaagtggtatatttggtccatggcctgcctaaccgatccaatcttgtcgaaacaaagggttgcgcttacaaaatccatttcgtttatctatgtaatagatgatattttcgacatgtacagttcacttgatgaactcattctcttcacccaggctgtctctagctgggaatatagtgctatagaaaaacttccagactccatgaagacatgctttaaagccctagataatatgatcaatgagtctagccatacgatctatcaaaagcgtggatggagccctttacactctcttcggaagacgtgggcaagtttgtgcgaagcctttttagtagaggcaaaatggtttgcatcaaggcacgtaccaaaggcaaaagaatacttggaaaatggtgtggtcagctcaggagtgcacgtagttctggttcatatatttgttctcttggatgaaaccagtctcacccaaaagacactggattttgtggaaaacatgccttccatcattacttctacagcatcgattcttaggctttgggatgactttggtagtgccaaggatgagaatcaagaaggacatgacggatcttatgtggagtgttacatgaaggaattaggaggcagtgttgaagatgcacgtgaggaaatgatggaaaagatttcagatgcatggaagtgcctcaacaaagaatgcatacttcgaaatccagcgtttccaccaccattcctcaaagcttctctcaatctcgcaagattagttcctttgatgtataattatgatcacaaccaacgcctaccccacttggaagaacatatcaaatctttgtta

SEQ ID NO: 1376 - DNA, 1683 bp, Artificial Sequence
  Description of Artificial Sequence Synthetic polynucleotide
atgttcacacaaatcttagtatcattatcttcaaatgacaacccaaatataattgttcgtcctacaaaaaaattccacccaaatatttggggagaacgattcttacattacaacatttcacaacaagatttgtattgtcaacaacaaaaggttgaggaattaaaagaagtggttagaagagaaatatttggagattattattatgatgcttggaaaaatcaattgaagataattgatgtggtagaacgtttgggattgtcttatcattttgaaactgaaatagaaaataagcttcaatgcatttacaacaaaaccaatattgatgatgaagatttacaccatgtttctattcgatttagacttctacggcaacatggatttagaatttcttctgcagatatatttgaaaaattcaaagatgaggatgggaactttaaaaaatgcttggtaagtgatacccttggtttgcttagcttatatgaagcctcacatttgagctgtgttggagaaaatatacttgatgaagcacttgctttcacaacaacacacctaaatgagtttttggcgaagaaaaaagaacaccatgaagacgatgatccattatcgaaagaaatatctcgagccctagagaggcccttaagaaagaccctagtaaatcttcatgcaaggcatttcatttcgatatatgaaaaagatgcctcacataacaaagtattgttacaacttgcaaaattagattttaatctattacaatgcatgcacaaaaaggagcttagtgaagtcagcaggtggtggaaagaatcagattttgtacacaaatttccatttgcaagagataggattgtggaattgtacctttggatattaggagtttattatgaaccccaatactgtttggcaagaaatattctagtcaaaatcatagctttggcctcaattgctgatgatatttatgatgcatatggtacacttgaagaacttgagttcctaactcatgcaattgaaaggtgggatataaattgtgtagataaactgaatcaggaatatatgcaaacattttataaagatgttttgaattgttatgaagagtttgagcaactgctagaaaaagaagaaacatacagagttcattatgcaaaagaagcgtttaagggattagttcaatctcatttgaatgaagctcgatgtttgcatagaggatacatcccaagttttgaagagtacttgaaatttgctttgggtaattgtggttactacatgttgatactaagctctttggttggaatgaaaactaatattgtaacaaaagaagtttttgagtggctctcgaaagatcgaaagattattagagcatctgttactatttgtaggctaatggatgacatagccgaacacaagtttgaggaagatgattatgaggaacattcagcagtggaatgctatatgattgaacatggcgtttgtgaagaggaagcctatgatgaactgaataagctaataatgaatgcatggaaagaaataaatgaagagtttttgaagcctactaaagtagcttcacctatattacttcgtgctcttaacttttcaagggttatggatcttattttcaaatttggagatggttatacacacgttggaaaaatcaccaaagatattgttgctgctttgcttattcatccaatt

SEQ ID NO: 1377 - DNA, 1320 bp, Artificial Sequence
  Description of Artificial Sequence Synthetic polynucleotide
tggttcctaaagaatgaggaaataaaaaactacatagaattcaactctgaatacttttttgttgcaatgctaaatctttacagagctactgatcttgcttttcatggtgaatttgagcttgatgaactaagaacattttcaagaaaattacttgagaaatctatttcagaaggagctcaacatacaaatccctttaagaaactgattgagcatgagttaagtcttccatggatggctcgactagatcacctcgaacataggttttttatcgaacaaactaataaagctttatggatgggaaagacatcttcacaaaggttatcaaagttttacaatgataaattaattcgtctagccactttaaactacgagttcaaacagtatatttacaagactgaacttgaacagttaacaaggtggtctaaagaatggggacttagtgaaatgggatttggtagagaaaaaagtacatacaattattttgcagtagcttgtgcttgctgttatttgccttatgattctccaattcgattgatgattacaaagggtgctataatagtaacggttgctgatgatttttttgatatgaaaggctctctattcactgatttaaaattcttcactaaggcaattcaaaggtggaataatgaagggttgagtggtgttagtaaaaagatatttgatgccctagataatcttgtaagggaaatgtcaactatgtaccttgaacaacaagaaaaaagtcatgaagacataaccagctacataagaaaaatttggtatgaaacgatttgttcatggcttactgaatctgagtggagtaaaagtggaattgtaccaaccatggaagaatattttaaagtttcaatgacttctgtagcaactcataccttgcttcttccagcttcatttcttctgaaaccgaccttaaaaaaatctcagttacaattaatggagtataaaagtgttactaaattggtcatgattatttgtcgtttgttgaatgacttacaaagttctgagagggaaagagaagaaggcaaaccaaactcaatcacagtttacatgaagaataattttgagatagagaataaggaagaagtagtaaagtacttgaaagagatattaaacgataagaagaaggagttccttgagcatgttttgattaatgacaatggttttaccaatcttcctaaagagtgtaggcttctacacttatcttgcttaaaagtattccaaatgttctttaactcttcaaatcgatatgattcagacactgaattgcttgaagacattaacaaagcgatttacattcctttg

SEQ ID NO: 1378 - DNA, 1590 bp, Artificial Sequence
  Description of Artificial Sequence Synthetic polynucleotide
aataacaaagagagttatgtgaagattattgagctgaaggaacaagtgaagaagaaattacttcatggtcttcatcctttggaaaatcctttagagacacttgaatatattgatgatattcaacgattggggttatcttattattttgaaaatgaaattgaacaagttttgaagcaatttcataataataataacaatcttcatcatgattttggtgataataacctttatgctgatgctcttcgatttcggttgcttagggaacaaggttataatattgcatgtgaggtatttgccaaatataagaatgagaaaggaaaatttaaggaatcaatttcgagtgacattcgaggaatgttgaacttgtatgaagcttcacaaatgagagttcgcagagaagaaatactagatgaagctcttatcttcacaactactcatcttcaatccttagtggaaacatcccaattgagctctccttatctagacctagtgaaacatgccctaatgcaccctatccgaaagagctttcaaagaagagaggcaagactttacatatcactttaccatcaactaccttctcacgaagagcttcttttaaccctcgccaaactagatttcaacttgcttcaacaactacatcaaaaggaattgaattatattacgaggtggtggaaggagtttgacttcaaaagtaagcaatctttttcaagagacagaatagtggagtgttacatttggaattatggagtatattttgaagctcaaacttcccaaattaggttgatgatgaccaagttgatttctttgcttactattattgatgattcttatgatagctatggtacacttgaagagctaaggccttttactgaagcatatatatatttacttaataaatatgatatgattatcaggtgggatatatctgctactgacaacttgccagagtacatgaaagtatgttacaaaaaatgtttagaatttttcaatgaaattgaagaatttacaaaggaaaattcatactgcgcgagttatattaagaaagggttgcaatgtatggttagagcttatagtaaggaagtccaatggttacacaataaatatatgccaacatttgatgaatattatccaattggacttgataatgctggatctgaagagctaatatcaatggccttttgtggaatgggagatgttgtaacaaaagaatctatggattggatattttctcaaccccaacctaagattatacgaactatgtcaattgttggaagacttatgaatgacattggctaccacaagtcggatcgaagaaaattatcaaaagatgtagtggcttctgctgtggagtgttacatcaaacaatatggtgtaacagatgaagaagctatagagaaacttaacgaacaagttaatgattcatggaaggatctcaatgaagatcttctctatccaatcgctattcctagaccactcttaatgagagttctcaatcttgtacgagtgaaccatgagatgtacagagaaggtgacggttttacacaaccaacattgctcaagaatttgattgatactctcatcatcaatccc

SEQ ID NO: 1379 - DNA, 1605 bp, Artificial Sequence
  Description of Artificial Sequence Synthetic polynucleotide
aatgttgttgttcgaccaacaacaaaatttcacccttctatttggggagatcgattcctccattacaatgtatcacaacaacacttggaatgcaaagaagaacgagtgaaggaattagttgaagttgtaaggaaagagataatgatatcattattatcatcattgaagttaattgatagtgttcaacgtttgggattatcacaccattttgagactcaaatagaacaaatgttggaaaaacatgatctaaactactacaaacatgatctacatcatgattctattatgtttagactattaagacaacatgggtttaaggtttcatcaagcatatttgagaaattcaaagacgagaaaggtaattttaagaaaagcttgataaccgatgtttcgggtttgcttagtttgtatgaggcttcctacttgagttatgttggagaaagcatactagatgaagcacttgctttcacaaccactcaccttaaggctattgtggcaaatagtaaagatcatccattatcacatcaaatatccaaagcattggaaaggccattaagaaagaccatagagaggcttcatgctaggttttatatctcaatctatgaaaaggatgcctctcataacaaagtattgctagaacttgcaaagttagatttcaatctacttcaatgtttccacaaaatggagcttagtgaaattatgaggtggtggaaaaagcatgactttgcaaacaaattcccttttgcaagagataggatggtggaactatatttttggatgttgggtatatattatgaacccaaatactctcgagcaagaaagctaacaaccaaaatctctgctttgatttcaatcacagatgatatatatgatgcatatggtacaattgatgagcttgagcttcttacccaagccatgcaaaggtgggacataaattttatagataaacttgagccagaatatttgaagacatattataaagcaatgttgacttcttatgaagaatttgaaaaagagtttacaaaggaagaattatacaaacatcaatatgcaaaagaagagatgaaaaaattgattcgagcttatttagaagaagctcgatggttgaacgatggatacttgccaagcttcgatgagcatttgagagtttcttatgtttcttgtgcttacacaacgttaatcgccacaagttatgtaggaatgtatgatattgtaacacatgaaactctaaattggctctccaaagaccctaaaattgtttcagcttccactctacttgctaggttcatggatgatattggttctcgcaagtttgagcaagagagaaatcacataccatctacagtggaatgttacatgaagcaatatggggtatcagaaaaagaggcaattgaagaactgaataaaagagtggtcaattattggaaagaaattaatgaagacttcattaggccaactgttatgcctttccctatcttagttcgtgttcttaatgttacaaaagtattggatcttctttacaaaaatggtgatgatcaatatacacatgttggaaaattgtttaaagaaagcattgctgctttacttatagatccaatccctgta

SEQ ID NO: 1380 - DNA, 1917 bp, Artificial Sequence
  Description of Artificial Sequence Synthetic polynucleotide
atggctgcattagtgtccactgtaagtagtattattaggtggaatagtaataataataataataataacttcactagatctgtgaaatcatgtttaagttcaaattaccataataataataatattattcataataaaactgtattaatgtccaccaataataataataataataatcagaagaatagttctcgaagatcagcaaactatcaacccccactttggcaatttgattatgtacaatcactttcttctcctttcaaggaaggagcatatgccaaaagagttgagaaagtaaaggaagaggtaagagtaatggtgaagagagcaaaagaggaggagaagcctttatctcaacttgagctaattgatgtaatgcaaagacttggaatctcttaccactttgagaatgaaattaatgacatattgaaagatatatataacaacaataatgtgtgcaacaccaacaataatgtgtatgccaattctcttgaatttagactcctaagacaacatggttatccggtgtctcaagaaatatttagtacgtgcaaagatgaaagaggcaattttatggtgtcttccaatgatatcaaaggaatgttatctttatatgaagcttcattctatttggtagaaaatgaagatggtattttggaagagacaagacacacaacaaagaaatatcttgaggaatacataatcatgatcatggaaaaacaacaatcattattagatcaaaataataataataatgataatgattatgattatgaactagtgagccatgcattggagcttccacttcattggagaatgttaagattggagagtaggtggtttattgatgtgtatgagaagagactagacatgaaccctactctacttatgttagctaaactagatttcaacattgtccaatcaatataccaagatgatcttaaacatgtcttcagctggtgggaaagcactggtatgggaaagaagttggaatttgcaagagatagaacaatggtgaatttcttatggacagtaggagttgcatttgagccacatttcaaaagttttagaagaatgattacaaaagtaaatgctttaattacagtaatagatgacatatatgatgtttatggtacactagatgaattggagctcttcactaatgcagttgagagatgggatattagtgctatggatgggctccctgagtatatgaagacatgtttctttgctttatacaatttcataaatgatcttccatttgatgtgttaaaaggggaagaaggcctccatataataaaagtccttcagaaatcgtgggcagatctttgcaaatcttatttaagagaagcaagatggtattatgatggatacacaccaagatttgaagagtacattgagaatgcatggatatcaatatcaggacatgttatactatcacatttatacttttttgtagtgaatccaaacaaggaagatgccttattaagtacttgctttgatggataccctaccataatacgacattcatcgatgattttacgtcttacagatgatcttgcaacttcaacggatgaactgaaaagaggcgatgtttctaaatcagtccaatgcaaaatgcatgaagatggcgtgtctgaagaagaagctcgtcaacatattaagttattaataagtgaaacatggaaacttattaataaagattacataaatttggatgatgatcatgatggtgatgactactctccaatgttctataagtctaataatactaataaggctttcattgaaatgtgtttaaaccttggtagaatggcacattgcatttatcaatatggagatggacatggaattcaagatcgccaaacaaaagatcatgtactatcattacttatttaccctattcctcttacccaa

SEQ ID NO: 1381 - DNA, 2361 bp, Artificial Sequence
  Description of Artificial Sequence Synthetic polynucleotide
atgtctcattcattgcattccaacattaggtgctcctcctcttcttcctcatcagcttctcccataccaagactaaatggtgcaaggaaaagcaacatagctgttttgaagtttgaggagaccaaagagagaataaagaagatgttcaataatgttgaactctctgtctcgtcttatgacactgcctgggtggcaatgatcccttcaaattctgggcatcaccctttctttcctcaatgtgtaaattggttactagagaatcagcactctgacggttcatggggtcttcctcatcgtcatcccttgcttatgaaagatgctcttttatctactttaactagtgtacttgcaatgaagcgatggggtgttggcgaagaacaaatagacaagggcttgtctttcatagaggaaaatatagcttcagccattgatgagaagcaatgttcacctattggatttgaagtaatatttcctacaatgattgattatgcgaaaaaatcagatctgaatattactcttgagccatccaattataatgacctaattcataacagaaaggtttcacttgaaagagcctcaggaagctactcagagggtcatgaagcatacttagtgtacatttcagaaggtcttggaaagccactggattgtgaaagagtcttcaagtaccaaaggaaaaatggttctctgtttaattctccatccaccacagctgttgcttttacaaaccatgggaatgctgactgtcttaagtatctctgctctgttgtagacaagtttggaagtgcagttccaacagtatatcctctagatatttatgctcgtctctcaatggtggacaatctccaaaggttggggatagaacgatttttcaaagaggaaatcagaagtgtactggatgaaacatacaaatattggctgcaaggagaggagtgtgttctcttagatgcttccacctgtgcaatggcatttcgtttattgcgtgttaatggatatgatgtatcttcagatccattaattcgattttcagaagattatatatacaattaccttgggaatcataccaaggatatagatgctatcttggaaatatttagagcatccgagatcatcatacacccaaatgaatcaatcttggagaagcaaaatttctgtacaagtcattttctagaacaagagttatacagcatttcaactaggggagataaactcaataaaaatattggcaaagtggtaagtgaagctcttaagattcctttctatgcaaacttggaacgactagcaagtagaagagccatagagtactatgacactgatagtacgaggattctgaaaacttcatattgttcctcaaatgttcggagcaaggatttcctaatattagcagttgaagacttcaacatgtgccaatcaatacaccgtgaagaactcaaaattctttctaggtgggttgtagagaataggttggacaagctaaagtttgctaggcagaagctggcatactgttacttttctgctgctgctactctttcttctcctgaattatctgatgcccgcatatcatgggcgaaaaatggggtacttactacagtggttgatgatttttttgatattggaggttcagaagaggaactagtaaacctcattcaattgttggaaaagtgggatgtagatgtcagtgttgattgtttgtcggagcaagttgaaattatttatttagcacttcatggcacaatctctgaaattggagaaaaggcgttcgtgtggcaaggacgtagtgtgacaaatcacataatagagatttggctggatttgctcaagtctatgttgaaggaagctcaatggttgaaatacgagaccgtcccaacaatagatgagtatatgaaaaatggatacatatcatttgccttaggacctatagtccttccagcactttacttagttggacctatgctttctgaggatgttacaagacatcctgagctccattatctatataaactcatgagcacttccggacgacttctcaacgatatccacggcttcaagagagaatccaaagaagggaaactgaatatagtgtcactaagcctactggcgcgtggttttggctctggttcttcttctgaagaagagatcattaatgaaatgaaaagtgctgtaaatggtaagaggagagaattgctgaggctagtttttaaggaaaaagatagcattgttccaagagcttgcaaggacttgttctggaaaatgagccaagtgttgcacctattttatgccaatgacgacggattcacctcaaatgagatgatcaatgtcgcaaaagcaataattgaggagcccataattgaaaataaattg

SEQ ID NO: 1382 - DNA, 1701 bp, Artificial Sequence
  Description of Artificial Sequence Synthetic polynucleotide
atgtcgtctcaaatcttagcaacctcatccaaaaatgacaatatacataaaattgttcggccaacaacaaattatcatccttctatttggggagaccgatttttgcattacgatattccaaaagaagaattggtgtataaacaaggcgaagttgaagaattgaaagaagtggtaagaaaggagatatttggagaattcttatgtgatgattggagtaatcgattgaagttaattgatgtggtgcaacgtttgggattgtcctatcattttgagagtgaaatacaaaatgagcttcaacacatttacaacaaaataagcattaatgattctaatttcgaacatgaaactttgcatgatgcttctattcggtttagacttctacgacaacatggatatagggtttctttagatatatttgacaagttcaaagatgagaatggcaactttaaagaatgtttggcgagtgacaccgttggtttgcttagcttatatgaggcctcacatttgagctgtgttggagaaaatttactagatgaagccctttctttcactaccaaacacctaactgaatttttggaaaataataaaaaagaacaccccaatgatgatccattatcaaaggaaatatctcgagccctagagaggcccttaagaaagaccctagtgaatctccatgcaaggtatttcatttcaatatatgaaaaagatgcctcacataacaaagtattgctacaacttgcaaagttagatttcaatctattacaatccatgcacaaaaaggagcttagtgaaatctcaaggtggtggaaggaattagacagtgcacacaactttccatttgcaagaaataggattgtggaactatatatttggatattaggagtctattatgaaccccaatactcttttgcaagaaatattttagtgaagatcatagcactttcctcaatcgctgatgatatttatgattcatatggtatatttgaagaacataagctccttattgaagcaattgataggtgggacaaaaattgtatggataaactccatccagaatacttgcagaaatattataagatacttttgcaatcttttgaggaatttgaacaagagtttgaaaaggaggaaacttacaaagtttactatggaaaagaaacgtttaaaagattattaagaggttattttgaggaagctcgatggttgaatgaaggatacatgccaagtttggaggagcatttaaaagtttctttggttacttctggttatttcatgttgatggcttgctctttagttggaatgaaaagtaacaatattgtaaccaaacaagttttcgagtggctctccaaagaccccaagattgttagggcaagcgctagtgtttgtaggtacatggatgacgtggctggtcacaagaatgagcaagagagaaatcatataccatctacaatagaatgttacatgaagcaatatggtgtatcagaagaagaagcatgtgatgaaatgaataggcgagtggttattgcatggaaagaaataaacgaagagtttctcaaaccaactgaagcagcttcacctatattagttcgtgctctgaatcttgctagggttatggatcttctctacaaaaatggtgataactatacacaagttggaaaagtcactaaagatagtgttgctgtcttgctcattgatccaattcca

SEQ ID NO: 1383 - DNA, 1587 bp, Artificial Sequence
  Description of Artificial Sequence Synthetic polynucleotide
aataacaaagagagttatgagaagattattgagatgaaggaacaagtgaagaataaattacttcatggtcttcatcctttggaaaaccctttggagacacttgaatatattgatgatattcaacgattggggttgtcttattattttgaaaatgaaattgaacaaaatttggagcaatttcataataattatcaaaatctaattgattttggtgataataacctttatgctgatgctctttgctttcggttgcttaggcaacaaggttataatattgcatgtgacatattcgacaagtacaagaatgaaaaaggaaaatttaaagaatcaatttcgagtgacattcgaggaatgttgaacttgtatgaagctgcacaaatgagagttcatggagagaaaatactagacgaagcacttatctttacaactactcatcttgaatcctcagttaaaacatgtcaattgagctctccttatctagacctagtgaaacatgccctaatgcaccctattcgaaagagcttacaaagaagagaggcaagactttacatatcactttatcatcaactaccttctcatgaggagattcttttaacacttgctaaattagatttcaacttgcttcaaaaactacatcaaaaggaactaagttacataacgaggtggtggaaggagtttgattacaaaagtaagcattcatttataaaagacagaatagtggagtgctatttctgggtttatggagtgttttttgaggcagaaacttcccaaatcagactaataatcaccaaattaattgctattctcacaataattgatgatgcttatgatagctttggtacacttgaagaactagagccttttactcaagcaatagaaaggtatactttataatatatatatatgattaggtgggatatatgtgccatggatactctgcctgagtacatgaaaatattttacatgaaacttttggagatctacaatgaaattgaacaattttctaaggaaagatcatactgccctagctatgctaagaaaggggtgcaatctctaattagagcttattttaaggaagccaaatggttacacacaaaatatataccaacattagatgaatatatgccagttggggttgatagtgcaggatcttttatgctcatttcaatggtttttattggaatgggagatattgttacaaaacattctatggattggatattttctaatcctcaacctaaaattatacaaactatggcaatagttggaagagttatgaatgacattggctaccataagtcggagcggaagaaatcatcaggagaaattgtggcttcaactgtggagtgttacatgaaacaatatggcgtgactggtgaagaagctatagagaaacttagcgaacaagttaaagattcatggaaagatctcaatgaagatcttctcaatccaatcactatccctaggccactcttaatgcaagttctaaagcttgtacgagtgaaccatgagatttatagagaaggagatggctttacacaacccactttgctcaagaatttgattcattctctcatcatcaatcca

SEQ ID NO: 1384 - DNA, 1695 bp, Artificial Sequence
  Description of Artificial Sequence Synthetic polynucleotide
atgtccactcaaatcttagcatcatcatcatcttcaaatgaaaacccaaataaaattgttcgtcctacaaaaacatttcatccacatatttggggagaacaattcttacattacaacatttcagaacaagatttgtattgtcaacaagaaaaggttgaggaattaaaagaagtggttagaagagaaatatttggagaatcatcaatatcatcatcagcttattatgatggttggaaaaatcaattgaagataattgatgtggtagaacgtttggcattgtcttatcattttgaaactgaaataggaaatatgatagaacaaatctacaacaaaaccaatattaatgatgaagatttacatcatgtttctgatcggtttagacttctacgacaacatggatttagaatttcttcggatatatttgaaaaattcaaagacgaggatggcaactttaaaaaatgtttggcaagtgatacccttggtttgcttagcttatatgaggcctcacatttgagttgtgttggagaaaatatacttgatgaagcacttgctttcacaacaacacacctaactgagtttttagcaaagaaaaaagaacaccatgatgacgatgatccattatcgaaagaaatatctcgagccctagagaggcccttaagaaagaccctgaatcttcatgcaaggcatttcatttcgatatatgaaaaagatgattcacataacaaagtattgttacaacttgcaaaattagatttcaatctattacaatgcatgcacaaaaaggagcttagtgaagtcagcaggtggtggaaagaatcagattttgtacacaaatttccatttgcaagagataggattgtggaattgtacctttggatattaggagtttattatgaaccccaatactgtttggcaagaaatattttaatcaaaatcatagctttggcctcaattactgattatgatgcatatggtacacttgaagaacttgagctcctaactcatgcaattgaaaggtgggatataaactgtgtggatacactcaatcaagaatacatgcaaacattttataaggacgttttgaattgttatgaagagtttgagcaagtgctagaaaaagaagtaacataaagagttcattatgcaaaagaagcgattaagggattagtccgatctcatttggatgaagctcgatgtttgcatagaggactcatcccatgttttgaagagtacttgaaatttgctttggataattgtgcttactacatgttgatactaagctctttggttggaatgaaaactaatattgtaacaaaagatgtttttgagtggctctccaaagatcgaaagattattagagcatctgttactatttgtaggctcatggatgacatagctgaacacaagtttgaggaagatgattatgaggaacattcagcagttgaatgctatatgattgaacatggtgtttgtgaagaagaagcctatgatgaactgaataagctaataattaatgcatggaaagaaataaatgaagagtttgtgaagccaactcaagtagcttcacctatattatttcgtgctcttaatttttctagggttatggatcttattttcaaatttggagatggttatacacacgttggaaaaatcactaaagatattgttgctgctttgcttattcatccaattcca

SEQ ID NO: 1385 - DNA, 1461 bp, Artificial Sequence
  Description of Artificial Sequence Synthetic polynucleotide
caagagttgaagcaagttacgagtacttcacttagaaccacaaaagacccttgtattctcttgaagcttattgattctatccaaaggttgggactcgaatatcacttcgaaaatgagattgaagaagctgtttctttcatttacgcacataatgatcaaactactagtaatgatctcttcatgacagcacttagatttcgtattcttaggcaacatggcttatttgttggctcagatgtgtttgacagatttagaggcagagatgggaaattcttggacagcttaagcagtaacaagcatggaattttgagtttgtatgaagcttcacaccttggaatggctgaagaaaatgttttggaggaagccaagagtttcaccaccaaaaggctaagatatttctcagctgggaaaatggataatactttgtttggtaagcaagtgaaacaatcactggaagttccactgtattggaggatgccaagatctgaagctaggaatttcattgatctctaccaaatggatgagacaaagagtgtgactttgcttgagttggctaagttggattataatctggtccaatctgtgcatcaaaatgagcttaaggagttgggaaggtggtgggaaaatttgggattcaagaaaaatctaccttttgcaagagatcgtgtggtggagaactatttgtgggccatgggaatcgtatctgagccacagttctccaaatgcaggataggcttaaccaagttcgtttgcatattaacagcaatagatgatgtttatgacatatatggatcattagatgagcttgaacttttcacaaatgcagtagaaagctgggacataagggcaataagggatgaatttcctctttatttgaaaacatgctatcttggcatgcttaattttggtaatgaagtgatcgatgatgttcttcaaaaccatggcttgaatatttcctcctacattaaagaagagtggttaaatctctgcaaatcatatcttgtagaagcaaggtggttttataatgattatacaccaagtttgaatgaatacttggaaaattcatctacttcagttggtggtcatgcagctatagttcatgcttgtattttactcttagatggctccatacccgaaaccttactggattacaatttcaaccattttcactctaagctgatttattggtcatctcttataacaaggcttagtgatgatttaggaacttcaaaggatgagcttaaaagaggagatgtgaagaaatcagtggagtgctacatggcagagaaaggaatatgggaagaagaagaagcaataaatcacataaaagaattgagaagaaattcatggaaaatggtgaacaaggagataataattgggaataattgcctgccaaaaataatggtgaagatgtgcttgaacatggcaagaactgctcaattcatattccaacatggtgat

SEQ ID NO: 1386 - DNA, 1692 bp, Artificial Sequence
  Description of Artificial Sequence Synthetic polynucleotide
agtactccaatattcaaagatcaaccagcagcaatcgtccgccgatcaggaaactacaaacccacattgtgggatgctcattttttccaatcactccaagtcatatatacggaggaaagctatgggaaaagaatcaatgagctgaaagaagatgtgagaagacttcttgagaaggagagtgaaaatcctttagttaagcttgaacacatcaatgatttgagcagacttggaatatcttatcactttgaagatcaaattaagacaatattaaacctgacatttaacaataataatgcattgtggaagaaagataatttgtatgccactgctcttcatttcaaactccttcgacaatatggatttagtcatgtatcttcagagattttcaatgccttcaaaaacgagcaaaaggagttcaaagagagtttgagtaaggatgtgaaaggaatggtatgtctatatgaggcttcattctattcatttagaggtgaacccatattagatgaagcaagagacttcacaaccaaacatctcaaacaatacttgatgacaaggcaaagtcaatttactagagttgaccatcatgatgatgatgatcatgatcttgtgaaattggtaaagcatgccttggatcttccactccattggagattgccaagattggaggctaggtggtttattgatatgtatgctgaaaggaattatgatatgaatccaacttttcttgactttgctaaacttgactataacttcgttcagtcagcatatcaaaaagagctcaaatatatttcaaggtggtggagtggttctagattgacagaaaggctaccatttgctgacagagtagtggaaattttctattctgcagtggcattgaaatatgaggcggaatttggatttgttagaactgtgatgacaaaaattggtctcttgctaacactcatggacgatatatatgatgtgtatggtacactagatgagcttcagctttttctagaggcaattgagaggtggaatataaatgaactggatcagctacccgattacatgaagatattgtttgttgctttttataacaatgtgaatgagatatcttattatgtcctcaaagaaaatgggattcacaccatcaaatatctgaagaaggcgttgggggatctgtgtaaatgctatatggaggaggcaaaatggtttcacagtggacacataccaagtttagaagaattcattgagaatggatggaattcaattacaatacctctttgtcttatttatcattattgcttaattacaacttcaattacagaacaagacatggaacacttgcttcaatatccaaccattttacgtgtctcaggaactgtttttcgatttatagatgatttgggaacctcatcagatgaacttgaaagaggagataatccttcatcaattcaatgctacatgcgtgaaaagggtgttagtgaaaatgaaagccgtgaacacatatggaatttaattagtgaaggatggaaggaaattaatgaagttaaagcttcaaactctccatactctcaagtgtttattgaatctaccattgattttgttagaggagcaatggagatgtatcacaagggtgatggttttggtactaatcaagaccgatatcttaagaacaaagttgtcaacatgttcttcgatccaattccaatt

SEQ ID NO: 1387 - DNA, 1584 bp, Artificial Sequence
  Description of Artificial Sequence Synthetic polynucleotide
aataacaaagagagttatgtgaagattattgagctgaaggaacaagtgaagaataaattacttcatggtcttcatcctttggaaaaccctttagagacacttgaatatattgatgatattcaacgattggggttatcttattatttcgaaaatgaaattgaacaagttttgaagcaatttcataataataacaatcttcatcacgattttggtgataacctttatgctgatgctcttcgctttcggttgcttagggaacaaggttataatagtgcatgtgaggtattcgccaaatataagaatgagaaaggaaaatttaaggaatcaattttgagtgacattcgaggaatgttgaacttgtatgaagcttcacaaatgagagtatgcggagaaaaaatactagatgaagcacttatcttcacaactactcatcttcagtccttagtggaaacatcccaattgagctctccttatttagacctagtgaaacatgccctaatgcaccctattcgaaagagcctacaaagaagagaggcaagactttacatatcacgttaccaccaactaccttctcacgaaaagcttcttttaactgccaaactagatttcaacttgcttcaacaactacatcaaaaggaattgaattatattacgaggtggtggaaggagtttgacttcaaaagtaagcaatctttttcaagagacagaatagtggagtgttacatttggaattatggagtatattttgaagctcaaacttcccaaattaggttgatgatgaccaagttgatttctttgcttactattattgatgattcttatgatagctatggtacacttgaagagctaaggccttttactgaagcatggatcatatatatataattatacttttattttgtcatatcattaatttttttttccacacatataataaaatatttaatatttattaaaaaaattcttttaattttatttttttaatttaaaaaagcaactaaatagttactttttcaaaactcccaaaaagcgaaaatttctagaaacgagatttttctagtttttttccattttcagtaattgttttgaatttcggagtgtatgctgacgtgacacaacgatatttgtataaataattttcttaaaagtatttttataaataaaatcacttttaagtataatattaaaaaaaaaaccctaatatatattgagtattgctataagaaatattatagtgtcatatattgaaaacatgtcatatattattattatagtgcaaataaattgcaagttcatccccataatcaaaaattatcaaaagatgtagtggcttctgctgtggagtgttacatcaaacaatatggtgtaacagatgaagaagctatagagaaacttaacgaacaagttaatgattcatggaaggatctcaatgaagatcttctctatccaatcgctattcctagaccactcttaatgagagttctcaatcttgtacgagtgaaccatgagatgtacagagaaggtgacggttttacacaaccaacattgctcaagaatttgattgatactctcatcatcaatccc

SEQ ID NO: 1388 - DNA, 1905 bp, Artificial Sequence
  Description of Artificial Sequence Synthetic polynucleotide
tctataatggctgcaatattaagtagtattattagcttgaatagtaataataataataataccttcactagatgtgtgaaatcatgtttaagttcaaaccataataattataatattattcataataaaactgtaataatgtccaccaataataataataatattaataatattatttctcgaagatcagcaaactatcaaccttcactttggcattttgattatgtacaatcactttctacccctttcaaggaaggagcatatgccaaaagagttgagaaagtaaaagaagaggtaagagtaatggtgaagagagcaaaagaggaggagaagcctttatctcaacttgagctaattgatgtaatgcaaagacttggaatctcttaccactttgagaatgaaattaatgacatattgaaagatatatataacaacaataatgtgtgcaacaccaacaaaaatgtctatgccaattctcttgaatttagactcctaagacaacatggttatccagtgtctcaagaaatttttagtacgtgcaaagatgaaagaggcaattttatggtgtcttccaatgatatcaaaggaatgttatctttatatgaagcttcattctatttggtagaaaatgaagatggcattttggaagagacaagacacacaacaaagaaatatcttgaggaatacataatcatgatcatggaaaaacaacaatcattattagatcaaaataataataattatgattatgataattttgattatgaactagtgagccatgcattggagcttccacttcattggagaatgttaagattggagagtaggtggtttattgatgtgtatgagaagagactagacatgaaccctactctacttaccttagctaaactagatttcaacattgtccaatcaatataccaagatgatcttaaacatgtcttcagctggtgggaaagcactgatatgggaaagaagttggaatttgcaagagatagaacaatggtgaatttcttatggactgtaggagttgcatttgagccacatttcaaaagttttagaagaatgattacaaaagtaaatgctttaattacagtaatagatgacatatatgatgtttatggtacactagatgaattggagctcttcactaatgcagttgagagatgggatattagtgctatggatgggctccctgagtatatgaagacatgttttcttgctttatacaatttcataaatgatcttccatttgatgtgttaaaaggagaagaaggcctccatataataaaattccttcagaaatcgtgggcagatctttgcaaatcttatttaagagaagcaagatggtattataatggatacacaccaagctttgaagagtacattgagaatgcatggatatcaatatcaggacctgttatactatcacatttatacttttttgtagtgaatccaatcaaggaagataccttattaagtacttgctttgatggataccctaccataatacgacattcatcgatgattttacgacttacagatgatctgggaacttcaacggatgaattgaaaagaggcgatgttcccaaatcaatccaatgcaaaatgtatgaagacggtatatctgaagaggaagctcgtcaacgtattaagttattaataagtgaaacatggaagcttattaataaagattacataaatttggatgatgatgatggtgatgactactctccaatgttctataagtctaataatattaataaggctttcattgaaatgtgtttaaaccttggtagaatggcacattgcatttatcaatatggagatggacatggaattcaagatcgccatacaaaagatcatgtactatcattacttattcaccctattcctcttacccaa

SEQ ID NO: 1389 - DNA, 1656 bp, Artificial Sequence
  Description of Artificial Sequence Synthetic polynucleotide
atgtcaaatattcaagtcttagcttcatttcaattaagtgacaaaattattgctcgtccaacaacaaattttcacccttctatttggggcgatcgattcctccattacaatgtttcagaacaagacttggtttgcaaacaagaaagaattgaagaattaatacaagttgtgaagaaagagatattatcttcaaatcatgatcaattgaagttgattgacaatctccaacgtttgggattatcacatcattttgagagtgaaattgagaaattgttggaacaattaagtatcggcactcatcatcaaaatcatcaagatctacatgatgcttctctttggtttagattattaagacaacatggatttaatgtttcatcaagtatatttgaaaaatttaaagacgatgaaggtaactttaagaaaagcttgataaccgatgtttcgggtttacttagcttgtatgaggcttcacacttgagttatgttggagaaagcatactagatgaagcacttgctttcacaaccactcacctcaaggctattgtggcaaatagtaaagatcatctattatcacatcaaatatccatagccttggaaaggcctctaagaaagaccatagagaggcttcatgctaggttttacatctcaatctatgaaaaggatgcctctcataacaaactattgctagagcttgcaaagttagacttcaatctacttcaatgtttccacaaaaaggaacttagtgaaattacgaggtggtggaaggagcatgagtttgcaaagaaattcccttttgcaagagataggattgtggaactgtatttttggatattgggtgtatattatgaacccaaatactctcgagcaagaaaacttctaaccaaagtcattgcattagcctcaatcacagatgatatttatgatgcatatggtactattgatgagcttcaacttcttaccaaagcaattcaaaggtgggacataaattgtctggataaacttgagccagaatatttaaagacatattataaggtaatgttggattcttatgaagaatttgaaaaggagcttaaaaaagaagaattatacaaacttgagtatgcaaaagaagagatgaaaagaattattggagcttattttgaagaagctcgatggttgagtgaaggatatttcccaagcttggatgagcatttgagagtttcatatgtttcttgtggttacgttttgttgatagccacaagttatgtaggaatgtatgatattgtaacacatgaaactctaaattggctctccaaagaccctaaaattgtttcagcttccactctacttgctaggttcatggatgatattggttctcgcaagtttgagcaaaagagaaatcacataccatctacagtagattgttacatgaaacaatatggggtatcagagaaagaggcaattaaagaacttaataaaagagtggacacccactggaaagaaattaatgaagactttattaggccaactgttgtgccttttcctatcttagttcgtgttcttaattttacaaaaatagttgatcttctttacaaagagggcgatgatcaatacacaaatgttggaaaagtgctcaaagaaagcattgctgctttgcttatagattcaatcccatta

SEQ ID NO: 1390 - DNA, 1698 bp, Artificial Sequence
  Description of Artificial Sequence Synthetic polynucleotide
atgtccactcaaatcttagcatcatcatcttcaaatgacaacccaaataaaattgttcgtcctacaaaaacatttcatccacatatttggggagaacgattcttacattacaacatttcacaacaagatttgtattgtcaacaacaaaaggttgaggaattaaaggaagtggttagaagagaaatatttggagaatcatcaatatcatcatcagcttattatgatgcttggaaaaatcaattgaagataattgatgtggtagaacgtttgggattgtcttatcattttgaaactgaaatagaaaataagcttcaatgcatttacaacaaaaccaatattgatgatgaagatttacaccatgtttctttacggtttagacttctacgacaacatggatttagaatttcttcggatatatttgaaaaattcaaagacgaggatgggaactttaaaaaatgcttggtaagtgatacccttggtttgcttagcttatatgaggctacacatttgagttgtgttggagaaaatatacttgatgaagcacttgctttcacaacaacacacttaaccgagtttttggcaaagaaaaaagaataccatgatgacgatgatccattatcaaaagaaatatctcgagccctagagaggcccttaagaaagaccctagtgaatcttcatgcaaggcatttcatttcgatatatgaaaaagatgattcacataacaaagtattgttacaacttgcaaaattagatttcaatctattacaatgcatgcacaaaaaggagctttgtgaagtcagcaggtggtggaaagaatcagattttgtacacaaatttccatttgcaagagataggattgtggaattgtacctttggatattaggagtttattatgaaccacaatactgtttggcaagaaatattttactcaaaatcatagctttggcctcaattgctgatgatatttatgatgcatatggtacatttgaagaacttgagatcctaactcatgcaattgaaaggtgggatataaattgtgtggataaactgaatcaagaatatatgcaaacattttataaggatgttttgaattgttatgaagagtttgagcaagtgctagaaaaagaagaacaatacaaagttcattatgcaaaagaagcgtttaagggattagtccgatctcatttggatgaagctcgatgtttgcatagaggactcatcccaagttttgaagagtacttgaaatttgctttggataattgtggttactacatgttgatactaagctctttggttggaatgaaaactaatattgtaacaaaagaagtttttgagtggctctcgaaagatcgaaagattattagagcatctgttactatttgtaggctcatggatgacatagctgaacacaagtttgaggaagatgattatgaggaacattcagcagtggaatgctatatgattgaacatggcgtttgtgaagaggaagcctatgatgaactgaataagctaataatgaatgcatggaaagaaataaatgaagagtttttgaagcctactaaagtagcttcacctatattacttcgtgctcttaacttttcaagggttatggatcttattttcaaatttggagatggttatacacacgttggaaaaatcaccaaagatattgttgctgctttgcttattcatccaatt

SEQ ID NO: 1391 - DNA, 1890 bp, Artificial Sequence
  Description of Artificial Sequence Synthetic polynucleotide
atggctgttcaccaattttctccatcaatagtatcatcactacctaccattagtacttataataataatcacttttgtagatttttcacaccaaaaacatctattagtcctatttcaaaaacaaaatcaaaatcaagcacttgctatcccatccaatgtactgtggtcaataatagtagccctagttctactattgttcgaagatcggccaattatgaacctccgatttggtctttcgattacattcaatctctttcaacccaatataagggagaatcctacactaggcaattaaataagttgaagaaagaagtgacaagaatgctccttggactggaaattaactctttagccctactcgagctaattgatacattgcaaaggcttggaatatcttaccattttaagaatgaaataaacactattttgaagaaaaaatacactgataattatattaacaataataatattattactaaccctaattataataatttatatgccattgctctcgaatttaggcttctacgccaacatggctatacagtacctcaagaaatttttaatgctttcaaggataagagagggaaatttaaaacatgcttgagtgatgatattatgggagtattgtgtttatatgaagcttcattctatgctatgaaacatgagaatattttggaggaaacaagaattttttcaactaaatgtcttaaaaaatacatggagaaaatggagaatgaggaagagaaaaaaatattattattagatgataataatattaatagtaatttgctattaattaatcacgctttcgagcttccgcttcattggagaataacaagatcggaagctaggtggttcattgacgaaatatatgagaaaaaacaagacatgaattcgactttgttcgagtttgcgaaattagattttaatatagtgcaatcaacacaccaagaagatttacaacatttatcgaggtggtggagggattgtaaacttggtgggaaattgaattttgctagagatagattgatggaagctttcttatgggatgttggactaaaattcgagggagaattcagctattttagaagaataaatgcaagattatttgtgcttataacaataattgatgatatttatgatgtatatggaactttggaggaattagagcttttcactagtgctgtcgaaagatgggatgtgaaattaatcaatgagttaccagattacatgaagatgcctttctttgttttacacaataccataaatgagatggggttcgatgtattagtacaacaaaactttgtcaacattgaataccttaagaaatcgtgggtagatttatgtaaatgttatttacaagaagcaaaatggtattatagtggataccaaccaacattggaagaatacactgagttgggttggctttcaataggagcatcagtaattcttatgcatgcttatttttgtttcacaaatcctataacaaaacaagacttgaaaagcttgcaattacaacatcattatcccaacataattaaacaagcatgcttgattacaaggcttgcagatgatttaggaacatcttcggatgaattgaatagaggcgacgttcctaaatcgattcaatgttacatgtacgataataatgctacagaagacgaagctcgtgaacacatcaagttcttgataagtgaaacatggaaggatatgaataagaaagatgaagatgagagttgtttatcagaaaattttgttgaagtttgcaaaaatatggctagaacagcactattcatatatgagaatggagatggacatggttctcagaatagtttatcaaaagaacgtatttcaaccttgattattactccaattaatattcctaaa

SEQ ID NO: 1392 - DNA, 1848 bp, Artificial Sequence
  Description of Artificial Sequence Synthetic polynucleotide
ttgggagttcatcaattttctccactttcattaatccttagttctacaaaattagcaagagcaagagaaagtaataaatcaagattatcaagtgcttgctaccccatccaatgtactatggtcaatagtacaaatattcctaataatactactatatttgataatcatattcatagaagatcagccaattatgaacctcccatttggtcttttgattacattcaatctctttcatcaagccaatataagggagaaacatgtacaagtagattgaatgagctagaagcaaatgtgaaagagatattgttagttgagatgaaatttaactctttagctcaacttgagttgattgatgcattgcaaagacttggattatcttatcgtttcgagactgaaataaacactattttgaataaaaattactccaataatgttatcaataaccctaattataatttgtacgccacagctctcgaatttaggcttctacgacaacatggttatgatgtatctcaagaaattttcaatgtgtttaaggatgagattacaagcaagttcaaggcaagaaggagtagcaaagatataattggagtattggctttatacgaagcttcattctatggcaaaaaaggcgaaagtattttggaagaagctagggtttcctcaaccaaatgtcttgaaaattatgcagcgacgatgacgatggagcaaagcaatccatcattattagttaatgattatgatgatgataataatatgttattattagtaaatcatgtcttggagcttccacttcattggagaataacaagatcagaagcaaggtggttcattgatttatatgaaaaaaatcataacatgaattctactttgcttgaatttgccaaattggattacaacatggtacaatcaatatatcaagaagatctaaaacatctctcaaggtggtggagccatacaaaacttggagagaaaatggatttctttagagttagaataatggagtgtttcttatggactgtgggagtaacatgtgagccagaacaaagctattatagaagaatgtctggaagattgtatgttctaataacaacaattgatgatatatatgatgtttatggaacattggaggaattagagcttttcactaatgctgttgagagatgggatgtgaaagcaatggatgatttaccagaatatatgaggatgcctttctttcttttacacaataccataaatgaaatggctttcgatgtgttaggagaccaaaatttcctcaacattaaattccttaagaaaacgtgggcagatttttgtaaacatcaattacaagaggcgaaatggtttcacagtggatacaaaccaacattcgaagaatacattaacaatgcatggatttcagtatcaggaccgattattcttattgatgcttatttctctcttacaaatcctgttacaaaagatgccataaatttactggaattaggttatccacccatattttaccacgcatccatgattctacgacttacagatgatctgggaacatcgcctgatgaaatgaaaagaggtgatattccaaaatcaattcaatgttacatgaacgatacaggtgtttctgaagatgaagctcgagatcatatgaagtttctaataagtgaattatggaaggaaataaataatgaagatgaaaatatggactctcctttctcaaaacaatttcttcaaaattgtaaaaatcttgctagaatatcacaatttatatatcaatatggagatggacatgcttctcaggatagtttatcaaaacaaagaatttcagaattgataattaatcatattccttct

SEQ ID NO: 1393 - DNA, 1785 bp, Artificial Sequence
  Description of Artificial Sequence Synthetic polynucleotide
gtattttttagaccaaaagtatatactaattataatattattaataataatgctacaaaatcaagattatcaagtgcatgctaccccatccaatgtgctgtggtcaatagttctaatgcaattattgatcgacgatctgccaactttgagccctccatttggtcttttgattatattcaatctcttacaagtcaatataagggagaaccttatacaagtcgagtgaaaaaacttgagagagatgtgaaaaagatgttagttgagatggaaaactctttagctcaacttgaactcattgatacattgcaaagacttggaatatcttaccgttttgagaatgaaataaattctattttgaacaaaaaatatgttaatattaataatcctaattataatttatatgccattgctcttcaatttaggcttctacgtcaacatggctatgcagtacctcaagagatttttaatcagttgaaggacgagatagaaaacatcaaaaaacacataaatggtaatgatatcatgggagtattagccttatacgaagcttctttttatgagaaaaaaggcgaaagtattttaaaggaagctagaattttcacaactaaatgtctcaaaaactacactataatgatatcagagcaaaaaaagttaatgattgataatgattatgattatgatatcgaagtagtgaatcatgccttagagctcccacttcatcggaggaccacaagaacagaagcaaagtggttcattgacgcatatgcgaaaaaacaagacatgaatcctatgttgcttgagttagccaaactcgatttcaacatagtacaatcaacacatcatgaagatctaaaacatatattcaggtggtggagacatactaaacttggagagaaattgaattttgcaagagatcgattgatggaatgtttcttatggaatattggaataagatttgagtcaaaattcagctattttagaacaaaaactgccaaattatttgagctagtaacatttatagatgatatatatgatgtttatggaacattggatgaattagagcttttcaccaaagctgttgagagatgggatgtgaaaatgataaatgagttaccagaatacatgaagatgccttatcttgttttacacaataccataaatgatatggtatttgaggtgttaagagaccaagaaatttccatcaacattcaataccttaagaaaacgtgggtcgatatgtgtaaaagtttcttgcaagaggcaaaatggtactatagtggatacacaccaacattggaagaatatattgaaaatggttggatttcagtaggagcaccagttattcttgtgcatgcttatttttttcacgcaaataataatcgtacaattacaaatactaaagagattttcgaatgcttggaatatggttattatcctgccattattcgtcacagtgccataatattacgatttacaaatgacctagcaacatcatcggaggaattgaaaagaggtgatgctccgacatcaattcaatgttacatgcaagaaaaaaatgtatctgaagaggaagctcgtgaacatgttaagtttttaataaatgaagcatggaaggagatgaataatgatgttggattatatccaatctcattgactgaagatgctacaaactttgctaagatgggatttttcatatatcaacatggtgatggtcatagttctcaagataatcaatccaaacaaaaaatttcatccttgattattgaacctattcccctatataca

SEQ ID NO: 1394 - DNA, 1833 bp, Artificial Sequence
  Description of Artificial Sequence Synthetic polynucleotide
ttgggagttcatcaattttctccactttcattaatccttaatactacaaaattagcaagagcaagtacattatcaagtgcttgctaccccatccaatgtactatggtcaatagtacaaatattactaataatactactatatttgataatcatattcatagaagatcagccaattatgaacctcccatttggtcttttgattacattcaatctctttcatcaagccaatataagggagaaacatgtacaagtagattgaatgagctagaggcaaatgtgaaagagatattgttagttgagatgaaatctaactctttagctcaacttgagtttattgatgcattgcaaagacttggattatcttaccgtttcgagactgaaataaacactattttaaatgaaaagtactccggtaatattatcgataaccctaattataatttgtacgccacagctctcgaattcaggcttctacgccatcatggttatgatgtatctcaagaaattttcaatgtgtttaaggatgagattacaagcaagttcaaggcaagaacgagtggcgaagatataattggagtattggctttatacgaagcttcattctatggcaaaaaaagtgaaagtattttggaagaagctagggtttcctcaatcgaatgtctcgaaaattacgtagcaactatgacgatggagcgaaacaaaccatcattattagtcaatgattatgatgatgataataatatgttattattagtgaatcatgccttggagcttccactttattggagaataacaagatcagaagcaaggtggttcattgatttatatgaaaaaaatcataacatgaattctactttgcttgaatttgccaaattggattacaacatggtacaatccatatatcaagaagatctaaaacatctctcaaggtggtggagccatacaaaacttggagagaaaatggatttctttagagatagattaatggagtgtttcttatggactgtgggaatagcatgtgagccagaaaaaagctattatagaagaatgtctggaagattatatgttctaataacaacaattgatgatatatatgatgtttatggaacattggaggaattagagcttttcactaatgcagttgagagatgggatgtgaaagcaatggatgatttaccagaatatatgaggatgcctttctttcttttacacaataccataaacgaaatggctttcgatgtgttaggacaccaaaatttcctcaacgttaaattccttaagaggacgtgggtagatttctgtaaacatcaattacaagaggcaaaatggtttcacagtggatacaaaccaacattcgaagaatacattaacaatgcatggatttcggtatcaggaccgattattcttatggatgcttatttctctcttacaaatcctgttacaaaagatgccataaatttactggaattaggttatccacccataatttaccatgcatccatgattctacgacttacagatgatctaggaacatcgaatgatgaaatgaaaagaggtgatattccgaaatcaattcaatgttacatgaacgatacaggtgtttctgaagatgaagctcgagatcatatgaagtttctaataagtgaattatggaaggaaataaataatgaagatgaaaatatggactctcctttctcaaaacaatttcttcaaaattgtaaaaatcttgctagaatatcacaatttatatatcaatatggagatggacatgcttctcaggatagtttatcaaaacaaagaatttcagaattgataattaatcatattccttct

SEQ ID NO: 1395 - DNA, 1791 bp, Artificial Sequence
  Description of Artificial Sequence Synthetic polynucleotide
gtattttatagaccaaaaatatatactaactataatattattaatggtggtacaaaatcaagattatcaagtgcatgctaccccatcatatgtgctgtggtcaatagttctaatgcaattattgatcgacgatctgccaactttgagccctccatttggtcttttgattatattcaatctcttacaagccaatataaggaagaatcttatacaggtcgagtcaaagaattggagatagatgtgaaaaagatgttaattgagatggaaaattctttaattcaacttgaactcattgacacattgcaaagacttggagtatcttatcgttttgaggatgaaataaatactattttgaaagaaaaatatattaatatgatcaataaccctaattataataatttatatgtcactgctcttgaatttaggcttctacgtcaacatggctatgcagtacctcaagagatttttaatcattttaaggacgagataggaaaaatcaaaacgaacatgattagtgatgatattatggaagtattggccttatatgaagcttcattctatgagaaaaaggaaagtattttagaagaagttagaattttcacaactcaatatctaaagaactacattataatgatatcacaagaaagaaagttaatgattgataatgataatgattatgattatgatatcgaaatagtgaatcacgctttggagctcccacttcattggaggactacaagaacagaagctaagtggttcatcgacgcgtatgagaaaaaacaagacatgaatcctattttgcttgagtttgctaaactcgatttcaacatgatacaatcaacacatcatgacgatctaaaacatatattcaggtggtggaggcatactaaacttggggagaaattgaattttgcaagagatcgattgatggaatgtttcttatggaaagttggaataagatttgagccaaaattcagttattttagaaaaacaactgccaaattatttgagctaataacagtaattgatgatatatatgatgtttatggaacattggatgaattagagcttttcaccaaagctattgagagatgggatatggaaatgataaatgagttaccagaatatatgaagatgccttatattgttttacacaataccattaatgaaatggtatttgaggtattaagagaccaacacatctccatcaacattcaataccttaagaaaacgtgggtagatatgtgtagaggttttttacaagaggcaaaatggtactatagtggatatacaccaacactagaagaatatattaaaaatggttggatttcagtgggagcaccggttctaattgtgcatgcttatttttctcatgcaaattataatcacacagttacaagtagtaaagagattttcgaatgctttgaacatggttattatcccgccataattcgtcatagtgccataatattacgacttacaaatgacctagcaacatcatcggaggaactgaaaagaggtgatgctccgacatcaattcaatgttatatgcaagaaaaaaaagtatctgaggagaaagctcgtgaacatattaagtttttaataagtgaaatatggaaagagatgaataatgatgttggattatatccaatctcattgattgaagatgctacaaattttgctaagatgggatttttcatgtatcagcatggtgatggtcatagttctcaagataatcaatccaaacaaaaaatttcatccttgattattgaacctattcccttatataca

SEQ ID NO: 1396 - DNA, 2152 bp, Artificial Sequence
  Description of Artificial Sequence Synthetic polynucleotide
gacttgtaatatcatcgaaattgaaagacacaggaaaaatataaaaataaataaaagataaacattatggacacacagagaaaattgcaagcagagcaactatcttgcccaaccaagtctcatgaactaatatttgatcataaatctgatcatcaaagacgttcggccaattatacgccaaccatttggaaatatgatttcctggagtctcttaacaacaaatatgatagtgaagagtacaaaaagctgtctgagaagcttatcgaggatgtgaggcatattattgttgaaactaaagacttaaagggtatgctagagctgattaacaccatcagaaaactaagcttgacatatcatttcgaagacgaagtcaagaaagttcttgacaaaatatcatcttccgattactactacaacaataatgacattaaagatcccctcgtaggagacgatctctatctcgctgcattatatttcaggcttcttaggcttcatggatatcatgtttcacaaggcatatttgttgggtacaatagtgtggattacaaaaaaggtggtgggacacataatattacatcaacagaggtgaaagtgatgatagagcttttagaggcctcacatgtagcttttgagtgtgaggagatgctaactgaggctaaagctttgatggaagaaaacctcaaaattgcctttcctgacaacggcaacaaatatcttcccaaacatgaggtggttcatgctttggaacttccatctcattggagggttcagtggtttgacgtaaaatggcaaatagaagcttatcgtcaaggtgacccggataccaacactacaactactactagtctcttggttgatttagccaaattaaactttaacatagttcaagccacacttcaaaaagatcttagggagttgtccagttggtggaagaatgtgggcctttcagagaagttggattttgcgagagataggctggtcgagagcttcatgtgtactgtgggactggcttttcagcctgaatacaaaagtctgagaaaatgtcttaccaaagtggtcaatttcatactcattgttgatgatgtttatgatgtttatggctcattggaagagctcagacacttcaccaatgccgttgataggtgggatgtacgggaaactgaaaaacttccagattgcatgaagatttgtttccaagcactctaccatactacatgtgaaattgctagtgaaattgagactaagaacggttgcaagttagttttacctcatctaaagggagcgtggacagacttttgtaaatcactattaatggaggcagaatggtaccacaaggggtatatcccatccattgaggagtatttaagcaatgcttggatttcatcttcgggccctcttcttcttcttcattcatatcttgctatgccaaatcagactaatacggcctcttctcttgacataagcaaagatcttgtctacaacatttctctcatcattcgactatgcaacgatttaggaacttcagcggctgaacaggaaagaggagatgctgcatcttctatcgtatgttatatgcaagaaacgaagagtagtgaagaggaggcccgaaagcatatccgagaaatgattagaaaaacatggaagaagatcaacaagacatgctttagtacttgcggcagtagttcactatcattatcatttatagatattgcccttaacactgctcgagtggcacatagcctctaccaatctggagatgccttcagtgctcaacatacagactataagactcatattctctccttactcgttcaccctctcattcccaataagtaaactagctaggatgatgagtcctttaatgggtcaccatgtgttttcttttttataattaatagaaaaatttaggtggttaagcagtttattacaggttatgttatgattaattattattattattattattattattatttgttttctttttcatagtaattaagtttaagttttttatgtacaactcttcaaagcacgtgaacgatgtctcctgtactttgaggaattgagaatgtgtgtttgtcgttggtggttgatcactatagctagttgttatgtatcgtggtgattttagtgtaatttatattcagataaa

SEQ ID NO: 1397 - DNA, 1761 bp, Artificial Sequence
  Description of Artificial Sequence Synthetic polynucleotide
aattataattttgttagtagtactacaagatcaagattatcaagatcaagtgcttgctaccccattcaatgtactgtagtcgatagttctaatccaatgatcgatcgacgatcgggcaactatgaaccatccatttggtcttttgattatattcagtctcttacaagccaatatgagggagaaccttatagaagtcgggtgaaagagatcgagggagatgtgaaaaggatgctagttgagatggaaaattctttagctcaacttgagctcattgacacattgcaaagacttggagtatcttatcgctttgagaatgaaataagtactattttgaaggaaaaatatgtcaatgttaataaccctaattataatttatacgccattgctcttgaatttagacttctacgtcaacatggttatgcagtacctcaagagatttttaatcattttaaggacgagacaggaaaattcaaggcaaacataattagtgatgatattatgggagtattggccttatatgaagcttcattctatgagaaaaaaggcgaaagtattttagaggaagctagaattttcataaaaaaatatctcgaagactacactataatgatatcgcaagaaaagaaattaatgattgataatgattatgattataatttcgaagtagtgaatcatgcattagagctcccacttcattggaggactacaagaatagaagctaagtggttcatcgacgcgtatgagaaaaaacaagacatgaatcctattttgcttgaatttgctaaactcgatttcaacatgatacaatcaacacatcatgatgatctaaaacatatattcaggtggtggaggcatactaaacttggggagaaattgaattttgcaagagatcgattgatggaatgtttcttatggaaggttggagtaagatttgagccaaaattcagttattttagaaaaacaactgccaaattatatgagctaataacattaattgatgatatatatgatgtttatggaacattggatgaattagagcttttcaccaaagctgttgagagatgggatgtgacaatgataaatgagttaccagaatacatgaagatgccttttcttgttttacacaatacaataaatgaaatagtatttgagatattaagagacaaagacatctccatcaacattcaataacttaagaaaacgtggatagatatgtgtagaggttttctacaagaagcaaaatggtactatagtggatacacaccaacattggaagaatatatagaaaatggttggatctcagtgggagcaccagttcttattgtgcatgcttatttttctcattcaaataataataaacttgtctaagtttcagatgaagaagtggaatgtcttaaagagggttatcccaatattgttcgacatggatccctaatattacgacttgcagatgatctaggaacatgttcggatgaaatgaaaagaggcgatgttcctacatcaattcaatgttacatgcatgatactggtgtttctgaagacgaagctcgtgagcacatcaagcatttgataagcgaatcatggaaggagatgaataatgaaaatggagatcataattcttctttctcgaaagaatttgttcatgtttgcaaaaatcttggtagagcgtcacaatttatataccaatatggagatggacatgcttctcaaaatactctatcaaaagatcgtatttttaaaattattattaatcctttcccc

SEQ ID NO: 1398 - DNA, 20 bp, Artificial Sequence
  Description of Artificial Sequence Synthetic primer
tgctacatgcgtgaaaaggg

SEQ ID NO: 1399 - DNA, 22 bp, Artificial Sequence
  Description of Artificial Sequence Synthetic primer
tgtgatacatctccattgctcc

SEQ ID NO: 1400 - DNA, 20 bp, Artificial Sequence
  Description of Artificial Sequence Synthetic primer
tttttttttttttttttttt